(19) United States
(12) Patent Application Publication
(10) Pub. No.: US 2023/0062119 A1
(43) Pub. Date: Mar. 2, 2023

(54) USE OF BIOMARKERS IN IDENTIFYING PATIENTS THAT WILL BE RESPONSIVE TO TREATMENT WITH A PRMT5 INHIBITOR

(71) Applicant: Merck Sharp & Dohme LLC, Rahway, NJ (US)

(72) Inventors: Benjamin Nicholson, Wayland, MA (US); Rachel Allison Altura, Westerly, RI (US); Razvan Cristescu, Newton, MA (US); David John Curtis, Camberwell, Victoria (AU); Ian Philip Street, Williamstown, Victoria (AU)

(73) Assignee: Merck Sharp & Dohme LLC, Rahway, NJ (US)

(21) Appl. No.: 17/783,938

(22) PCT Filed: Dec. 16, 2020
(86) PCT No.: PCT/US2020/065341

§ 371 Date: Jun. 9, 2022

Related U.S. Application Data
(60) Provisional application No. 62/949,250, filed on Dec. 17, 2019.

(51) Int. Cl. C12Q 1/6886

(52) U.S. Cl. CPC C12Q 1/6886; C12Q 2600/106; C12Q 2600/158; C12Q 2600/156

(57) ABSTRACT

The present invention includes methods of identifying a patient who will likely be responsive to treatment with a protein arginine N-methyltransferase 5 inhibitor, or a pharmaceutically acceptable salt thereof, and methods of treating the same.

### BACKGROUND OF THE INVENTION

PRMT5 (aka JBP1, SKB1,1BP72, SKB1his and HRMTIL5) is a Type II arginine methyltransferase, and was first identified in a two-hybrid search for proteins interacting with the Janus tyrosine kinase (Jak2) (Pollack et al., 1999). PRMT5 plays a significant role in control and modulation of gene transcription. Inter alia, PRMT5 is known to symmetrically methylate histone H3 at Arg-8 (a site distinct from that methylated by PRMT4) and histone H4 at Arg-3 (the same site methylated by PRMT1). PRMT5 has been reported to perform diverse roles including but not limited to impacting cell viability, sternness, DNA damage repair and RNA splicing (Clarke et al Mol Cell (2017), Chiang et al Cell Rep (2017), Gerhart et al Sci Rep (2018)). Specifically, inhibition of PRMT5 induces alternative splicing of the negative regulator of p53, MDM4 resulting in increased expression of the short isoform of MDM4 (MDM4-S), decreased expression of the full-length isoform (MDM4-FL) and increased p53 activity (Gerhart el al Sci Rep (2018)). Most of the physiological functions of p53 are attributable to its role as a transcriptional activator, responding to agents that damage DNA. p53 status is wild type in approximately half of human cancer cases. These include 94% in cervix, 87% in blood malignancies, 85% in bones and endocrine glands, and 75% of primary breast cancer. Restoration of p53 in cancer cells harboring wild type p53, by way of inhibiting mechanisms that suppress its function leads to growth arrest and apoptosis, and is regarded as a potentially effective means of tumor suppression.

In response to DNA damage caused by a variety of agents, including doxorubicin, camptothecin and UV light, and also in response to treatment with Nutlin-3, knockdown of PRMT5 results in an increase in sub-G1 population and concomitant reduction in G1 cells and, in the presence of p53, a significant increase in apoptosis. Knockdown of PRMT5 also resulted in an increased level of p21, a key p53 target gene that regulates cell cycle arrest during the p53 response and MDM2, a p53 E3 ubiquitin ligase, but not PUMA, NOXA, AlP1 & APAF1, p53 target genes linked to apoptosis.

Knockdown of PRMT5 (but not PRMT1 or CARM1/PRMT4) results in decreased p53 stabilisation, decreased basal p53 levels, decreased p53 oligomerisation, and also decreased expression of elF4E a major component of translational machinery involved in ribosome binding to mRNA. Indeed, elF4E is a potent oncogene, which has been shown to promote malignant transformation in vitro and human cancer formation.

The role of PRMT5 in the DNA damage response has been explored with groups reporting a role for PRMT5 in regulating high fidelity homologous recombination mediated DNA repair in both solid (Clarke et al., Mol Cell (2017)) and hematological tumor models (Hamard et al., Cell Rep (2018)).

PRMT5 is aberrantly expressed in around half of human cancer cases, further linking this mechanism to cancers. PRMT5 overexpression has been observed in patient tissue samples and cell lines of Prostate cancer (Gu et al., 2012), Lung cancer (Zhongping et al., 2012), Melanoma cancer (Nicholas et al., 2012), Breast cancer (Powers et al., 2011), Colorectal cancer (Cho et al., 2012), Gastric cancer (Kim et al., 2005), Esophagus and Lung carcinoma (Aggarwal et al., 2010) and B-Cell lymphomas and leukemia (Wang, 2008). Moreover, elevated expression of PRMT5 in Melanoma, Breast and Colorectal cancers has been demonstrated to correlate with a poor prognosis.

Lymphoid malignancies including chronic lymphocytic leukemia (CLL) are associated with over-expression of PRMT5. PRMT5 is over-expressed (at the protein level) in the nucleus and cytosol in a number of patient derived Burkitt's lymphoma; mantle cell lymphoma (MCL); in vitro EBV-transformed lymphoma; leukemia cell lines; and B-CLL cell lines, relative to normal CD19+ B lymphocytes (Pal et al., 2007; Wang et al., 2008). Intriguingly, despite elevated levels of PRMT5 protein in these tumor cells, the levels of PRMT5 mRNA are reduced (by a factor of 2-5). Translation of PRMT5 mRNA is, however, enhanced in lymphoma cells, resulting in increased levels of PRMT5 (Pal et al., 2007; Wang et al., 2008).

In addition to genomic changes, CLL, like almost all cancers, has aberrant epigenetic abnormalities characterized by global hypomethylation and hot-spots of repressive hypermethylation of promoters including tumor suppressor genes. While the role of epigenetics in the origin and progression of CLL remains unclear, epigenetic changes appear to occur early in the disease and specific patterns of DNA methylation are associated with worse prognosis (Chen et al., 2009; Kanduri et al., 2010). Global symmetric methylation of histones H3R8 and H4R3 is increased in transformed lymphoid cell lines and MCL clinical samples (Pal et al., 2007), correlating with the overexpression of PRMT5 observed in a wide variety of lymphoid cancer cell lines and MCL clinical samples.

PRMT5 is therefore a target for the identification of novel cancer therapeutics.

Hemoglobin is a major protein in red blood cells and is essential for the transport of oxygen from the lungs to the tissues. In adult humans, the most common hemoglobin type is a tetramer called hemoglobin A, consisting of two α and two β subunits. In human infants, the hemoglobin molecule is made up of two α and two γ chains. The gamma chains are gradually replaced by β subunits as the infant grows. The developmental switch in human ß-like globin gene subtype from foetal (γ) to adult (ß) that begins at birth heralds the onset of the hemoglobinopathies ß-thalassemia or sickle cell disease (SCD). In ß-thalassemia the adult chains are not produced. In SCD, a point mutation in the coding sequence in the ß globin gene leads to the production of a protein with altered polymerisation properties. The observation that increased adult γ-globin gene expression (in the setting of hereditary persistence of foetal hemoglobin (HPFH) mutations) significantly ameliorates the clinical severity of ß-thalassemia and SCD has prompted the search for therapeutic strategies to reverse γ-globin gene silencing. To date, this has been achieved through pharmacological induction, using compounds that broadly influence epigenetic modifications, including DNA methylation and histone deacetylation. The development of more targeted therapies is dependent on the identification of the molecular mechanisms underpinning foetal globin gene silencing. These mechanisms have remained elusive, despite exhaustive study of the HPFH mutations, and considerable progress in many other aspects of globin gene regulation.

PRMT5 plays a critical role in triggering coordinated repressive epigenetic events that initiate with dimethylation of histone H4 Arginine 3 (H4R3me2s) and culminate in DNA methylation and transcriptional silencing of the γ-genes (Rank et al., 2010). Integral to the synchronous establishment of the repressive markers is the assembly of a PRMT5-dependent complex containing the DNA methyltransferase DNMT3A, and other repressor proteins (Rank et al., 2010). DNMT3A is directly recruited to bind to the PRMT5-induced H4R3me2s mark, and loss of this mark through shRNA-mediated knock-down of PRMT5, or enforced expression of a mutant form of PRMT5 lacking methyltransferase activity leads to marked upregulation of γ-gene expression, and complete abrogation of DNA methylation at the γ-promoter. Treatment of human erythroid progenitors with non-specific methyltransferase inhibitors (Adox and MTA) also resulted in upregulation of γ-gene expression (He Y, 2013). Inhibitors of PRMT5 thus have potential as therapeutics for hemoglobinopathies such as ß-thalassemia or Sickle Cell Disease (SCD).

There is a need for biomarkers that can be used to predict which patients are amenable to treatment with specific therapies. Specifically, there is a clear need for predictive biomarkers to determine whether a particular patient's PRMT5 mediated disease has a high likelihood to respond to treatment with a PRMT5 inhibitor in a patient with cancer or other diseases mediated by PRMT5. It is, therefore, an object of this invention to provide predictive biomarkers to select patients likely to respond to treatment with a PRMT5 inhibitor.

### SUMMARY OF THE INVENTION

The present invention includes methods for identifying a patient who will be responsive to treatment with a protein arginine N-methyltransferase 5 inhibitor and methods for treating the same.

The present invention relates to the identification of selection biomarkers whose expression level is useful for identifying, evaluating, and classifying patients responsive to a therapeutically effective dose of a protein arginine N-methyltransferase 5 inhibitor.

The present invention includes a method for treating a patient with a protein arginine N-methyltransferase 5 inhibitor after evaluating a biological sample from the patient for the presence of at least one selection biomarker.

### DETAILED DESCRIPTION OF THE INVENTION

As recognized by various cancer researchers, it is becoming important to identify potential responder biomarker(s) useful in predicting the therapeutic efficacy of an anti-cancer agent, e.g., PRMT5 inhibitor, particularly for use in clinical trials and for the design of treatment regimes. Analysis of expression responder biomarker(s) are considered to be more feasible and less burdensome for patients, because the number of samples needed for the analysis is fewer compared to conventional biomarker analysis.

The present invention relates to the discovery of a selection of biomarkers which have utility in predicting a patient's response to a treatment protocol comprising a PRMT5 inhibitor. The present invention comprises a method of identifying a patient who is likely to be responsive to treatment with a protein arginine N-methyltransferase 5 (PRMT5) inhibitor comprising evaluating a biological sample from the patient for the presence of any of the following: FLT3 internal tandem duplication (ITD), NPM1 mutation, DNMT3A mutation, SRSF2, SF3B1, ZRSR2, wherein the presence of any said mutation or alteration indicates a higher likelihood for said patient to be responsive to treatment with said PRMT5 inhibitor than in the absence of said mutation or alteration.

In one embodiment, the present invention comprises a method of identifying a patient diagnosed with cancer for treatment with a protein arginine N-methyltransferase 5 (PRMT5) inhibitor comprising:

evaluating a biological sample from the patient for the presence of at least one of the following:
a) a FLT3 internal tandem duplication, or
b) a mutation in NPM1 or DNMT3a; or
c) a mutation in any of the following splicing genes SRSF2, ZRSR2, or SF3B1.

In one embodiment, the present invention comprises a method of identifying a patient diagnosed with cancer for treatment with a protein arginine N-methyltransferase 5 (PRMT5) inhibitor comprising:
a) obtaining a biological sample comprising cancer cells from a patient diagnosed with cancer;
b) measuring the gene expression level of a predictive biomarker of PRMT5 inhibitor responsiveness in the biological sample;
c) wherein the predictive biomarker is selected from:
   i. a FLT3 internal tandem duplication; or
   ii. a mutation in NPM1 or DNMT3a; or
   iii. a mutation in any of the following splicing genes SRSF2, ZRSR2, or SF3B1;
d) identifying a patient predicted to be responsive to treatment with a PRMT5 inhibitor,
e) administering an effective amount of a PRMT5 inhibitor, or a pharmaceutically acceptable salt thereof, to the patient, if the predictive biomarker is present.

In a further embodiment, the present invention does not include a patient with a mutation in TP53 gene as the patient will likely not respond to a PRMT5 inhibitor.

A further embodiment of the present invention comprises a method of identifying a patient diagnosed with cancer predicted to be responsive to a treatment with protein arginine N-methyltransferase 5 (PRMT5) inhibitor, wherein the cancer is Myelodysplastic syndrome (MDS) comprising:
a) obtaining a biological sample comprising cancer cells from a patient diagnosed with MDS;
b) measuring the gene expression level of a predictive biomarker of PRMT5 inhibitor responsiveness in the biological sample;
c) wherein the predictive biomarker is selected from:
   i. a FLT3 internal tandem duplication; or
   ii. a mutation in NPM1 or DNMT3a; or
   iii. a mutation in any of the following splicing genes SRSF2, ZRSR2, or SF3B1;
d) identifying a patient predicted to be responsive to treatment with a PRMT5 inhibitor,
e) administering an effective amount of a PRMT5 inhibitor, or a pharmaceutically acceptable salt thereof, to the patient, if the predictive biomarker is present.

A further embodiment of the present invention comprises a method of identifying a patient diagnosed with the cancer Myelodysplastic syndrome (MDS) predicted to be responsive to a treatment with protein arginine N-methyltransferase 5 (PRMT5) inhibitor, comprising:
a) obtaining a biological sample comprising cancer cells from a patient diagnosed with MDS;
b) measuring the gene expression level of a predictive biomarker of PRMT5 inhibitor responsiveness in the biological sample;
c) wherein the predictive biomarker is selected from:
   i. mutation in DNMT3a; or
   ii. mutation in any of splicing genes SRSF2, ZRSR2, or SF3B1; and
d) identifying a patient predicted to be responsive to treatment with a PRMT5 inhibitor,
e) administering an effective amount of a PRMT5 inhibitor, or a pharmaceutically acceptable salt thereof, to the patient, if the predictive biomarker is present.

In one embodiment, the present invention comprises a method of identifying a patient who is likely to be responsive to treatment with a protein arginine N-methyltransferase 5 (PRMT5) inhibitor comprising:

evaluating a biological sample from the patient for the presence of at least one of the following:
a) a mutation in DNMT3a; or
b) a mutation in any of the following splicing genes SRSF2, ZRSR2, or SF3B1; wherein the patient has Myelodysplastic syndromes (MDS).

In one embodiment, the present invention comprises a method of identifying a patient that is likely to be responsive to treatment with a protein arginine N-methyltransferase 5 (PRMT5) inhibitor comprising:

evaluating a biological sample from the patient for the presence of at least one of the following:
a) FLT3 internal tandem duplication;
b) mutation in NPM1 or DNMT3a; or
c) mutation in any of splicing genes SRSF2, ZRSR2, or SF3B1; and
   wherein the patient has acute myeloid leukemia (AML).

A further embodiment, the present invention comprises a method of identifying a patient diagnosed with cancer predicted to be responsive to a treatment with protein arginine N-methyltransferase 5 (PRMT5) inhibitor according to claim 1, wherein the cancer is acute myeloid leukemia (AML) comprising:
a) obtaining a biological sample comprising cancer cells from a patient diagnosed with AML;
b) measuring the gene expression level of a predictive biomarker of PRMT5 inhibitor responsiveness in the biological sample;
c) wherein the predictive biomarker is selected from:
   i. a FLT3 internal tandem duplication; or
   ii. a mutation in NPM1 or DNMT3a; or
   iii. a mutation in any of the following splicing genes SRSF2, ZRSR2, or SF3B1;
d) identifying a patient predicted to be responsive to treatment with a PRMT5 inhibitor,
e) administering an effective amount of a PRMT5 inhibitor, or a pharmaceutically acceptable salt thereof, to the patient, if the predictive biomarker is present.

In one embodiment, the present invention includes a method of identifying a patient who is likely to be responsive to treatment with a protein arginine N-methyltransferase 5 (PRMT5) inhibitor comprising:

evaluating a biological sample from the patient for the presence of at least one of the following:
a) FLT3 internal tandem duplication
b) mutation in NPM1 or DNMT3a; or
c) mutation in splicing genes SRSF2, ZRSR2, or SF3B1;
d) wherein the patient does not have a mutation in TP53 gene; and wherein the patient has AML.

In one embodiment, the present invention includes a method of treating a patient diagnosed with cancer predicted to be responsive to treatment with a protein arginine N-methyltransferase 5 (PRMT5) inhibitor after evaluating a biological sample from the patient for the presence of at least one of the following:
a) FLT3 internal tandem duplication;
b) mutation in NPM1 or DNMT3a; or
c) mutation in splicing genes SRSF2, ZRSR2, or SF3B1; and
   comprising administering an effective amount of a PRMT5 inhibitor, or a pharmaceutically acceptable salt thereof, to the patient, if the predictive biomarker is present.

In a further embodiment, the patient has AML.

In a further embodiment, the patient has MDS.

In one embodiment, the present invention comprises a method for treating a PRMT5 associated cancer patient in need of treatment thereof, after evaluating a biological sample from the patient for the presence of at least one of the following:
a) a FLT3 internal tandem duplication; or
b) a mutation in NPM1 or DNMT3a; or
c) a mutation in any of the following splicing genes SRSF2, ZRSR2, or SF3B1;
   comprising administering an effective amount of a PRMT5 inhibitor, or a pharmaceutically acceptable salt thereof, to the patient, if the predictive biomarker is present.

In one embodiment, the present invention comprises a use of a PRMT5 inhibitor for use in the treatment of cancer in a patient in need thereof who has the presence of at least one of the following:
a) a FLT3 internal tandem duplication; or
b) a mutation in NPM1 or DNMT3a; or
c) a mutation in any of the following splicing genes SRSF2, ZRSR2, or SF3B1;
   comprising administering to the patient a therapeutically effective amount of the PRMT5 inhibitor or a pharmaceutically acceptable salt thereof.

In a further embodiment, the present invention comprises a PRMT5 inhibitor for use in the treatment of cancer in a patient in need thereof, wherein the patient is defined by:
a) assaying a biological sample from a patient to determine if a patient has a predictive biomarker;
b) wherein the predictive biomarker is the presence of at least one of the following:
   i. a FLT3 internal tandem duplication; or
   ii. a mutation in NPM1 or DNMT3a; or
   iii. a mutation in any of the following splicing genes SRSF2, ZRSR2, or SF3B1,
c) comprising administering a therapeutically effective amount of the PRMT5 inhibitor, or a pharmaceutically acceptable salt thereof, to the patient if the predictive biomarker is present.

In another embodiment, the present invention comprises the use of a PRMT5 inhibitor, or a pharmaceutically acceptable salt thereof, for the manufacture of a medicament for treating cancer in a patient in need thereof who has the presence of at least one of the following:
a) a FLT3 internal tandem duplication; or
b) a mutation in NPM1 or DNMT3a; or
c) a mutation in any of the following splicing genes SRSF2, ZRSR2, or SF3B1.

In another embodiment, the present invention comprises the use of a PRMT5 inhibitor, or a pharmaceutically acceptable salt thereof, for the manufacture of a medicament for treating cancer in a patient in need thereof, comprising:
a) assaying a biological sample from a patient to determine if a patient has a predictive biomarker; and
b) administering a therapeutically effective amount of a PRMT5 inhibitor, or a pharmaceutically acceptable salt thereof, to the patient if the predictive biomarker is present,
c) wherein the predictive biomarker is the presence of at least one of the following:
   i. a FLT3 internal tandem duplication; or
   ii. a mutation in NPM1 or DNMT3a; or
   iii. a mutation in any of the following splicing genes SRSF2, ZRSR2, or SF3B1.

In a particular embodiment, the present invention comprises an internal tandem duplication (ITD) typically ranging from 15-300 base pairs in the juxtamembrane region of fms-related kinase 3 (FLT3). In a preferred embodiment, the mutation is a FLT3 ITD.

In a particular embodiment, the present invention comprises a W288Cfs*12, L287fs, W290Sfs*5, or W288Cfs*7 mutation or alteration on the NPM1 gene.

In a preferred embodiment, the present invention comprises a W288Cfs*12 or L287fs mutation or alteration on the NPM1 gene.

In a preferred embodiment, the gene is NPM1 and the mutation is selected from the group consisting of W288Cfs*12, L287fs, W290Sfs*5, or W288Cfs*7.

In a preferred embodiment, the gene is NPM1 and the mutation is W288Cfs*12.

In a preferred embodiment, the gene is NPM1 and the mutation is L287fs

In a preferred embodiment, the gene is NPM1 and the mutation is W290Sfs*5.

In a preferred embodiment, the gene is NPM1 and the mutation is W288Cfs*7.

In a particular embodiment, the present invention comprises a R882C, R882H, R720H, Y592*, E229*, or V716D mutation or alteration on the DNMT3A gene.

In a preferred embodiment, the present invention comprises a R882 mutation or alteration on the DNMT3A gene.

In a preferred embodiment, the gene is DNMT3A and the mutation is selected from the group consisting of R882C, R882H, R720H, Y592*, E229*, V716D.

In a preferred embodiment, the gene is DNMT3A and the mutation is R882C.

In a preferred embodiment, the gene is DNMT3A and the mutation is R882H.

In a preferred embodiment, the gene is DNMT3A and the mutation is R8 R720H 82C.

In a preferred embodiment, the gene is DNMT3A and the mutation is Y592*.

In a preferred embodiment, the gene is DNMT3A and the mutation is E229*.

In a preferred embodiment, the gene is DNMT3A and the mutation is V716D.

In a particular embodiment, the present invention comprises a P94_P95insR, P95H/L/R, P95T, P95fs, P95_R102del, or P107H mutation or alternation on the SRSF2 gene.

In a preferred embodiment, the gene is SRSF2 and the mutation is selected from the group consisting of P94_P95insR, P95H/L/R, P95T, P95fs, P95_R102del, or P107H.

In a particular embodiment, the present invention comprises a P95H/L/R mutation or alteration on the SRSF2 gene.

In a particular embodiment, the present invention comprises a loss of function (LOF) mutation on the ZRSR2 gene.

In a particular embodiment, the present invention comprises a A284T, D586H, E592K, E622D, Y623C, R625C/G/H/L, N626D/I/S/Y, H662D/Q/Y, T663I, K666E/M/N/T/Q/R, K700E, V701F, I704F, G740E, K741T, G742D, D781G, E902K, or R957Q mutation or alteration on the SF3B1 gene.

In a preferred embodiment, the gene is SF3B1 and the mutation is A284T, D586H, E592K, E622D, Y623C, R625C/G/H/L, N626D/I/S/Y, H662D/Q/Y, T663I, K666E/M/N/T/Q/R, K700E, V701F, I704F, G740E, K741T, G742D, D781G, E902K, or R957Q.

In a particular embodiment, the present invention comprises a E622D, R625C/G/H/L, H662D/Q/Y, K666E/M/N/T/Q/R, K700E, or G742D mutation on the SF3B1 gene.

In a preferred embodiment, the gene is SF3B1 and the mutation is E622D.

In a preferred embodiment, the gene is SF3B1 and the mutation is R625C/G/H/L.

In a preferred embodiment, the gene is SF3B1 and the mutation is H662D/Q/Y.

In a preferred embodiment, the gene is SF3B1 and the mutation is K666E/M/N/T/Q/R.

In a preferred embodiment, the gene is SF3B1 and the mutation is K700E.

In a preferred embodiment, the gene is SF3B1 and the mutation is G742D.

In a particular embodiment, the present invention includes a method for treating a patient with a PRMT5 inhibitor after evaluating a biological sample from the patient for the presence of at least one of the following:
a) a FLT3 internal tandem duplication; or
b) a mutation in NPM1 or DNMT3A; or
c) a mutation in any of the following splicing genes SRSF2, ZRSR2, or SF3B1.

The present invention comprises a PRMT5 inhibitor, wherein the PRMT5 is one of the following:
(1R,2S,3R,5R)-5-(((2-amino-3-bromoquinolin-7-yl)oxy)methyl)-3-(4-chloro-7H-pyrrolo[2,3-d]pyrimidin-7-yl)-1-methylcyclopentane-1,2-diol,
(1R,2S,3R,5R)-5-(((2-amino-3-bromoquinolin-7-yl)oxy)methyl)-3-(4-amino-7H-pyrrolo[2,3-d]pyrimidin-7-yl)-1-methylcyclopentane-1,2-diol,
(2R,3S,4R,5R)-2-(((2-amino-3-bromoquinolin-7-yl)oxy)methyl)-5-(4-amino-7H-pyrrolo[2,3-d]pyrimidin-7-yl)-3-ethynyltetrahydrofuran-3,4-diol,
(2R,3S,4R,5R)-2-(2-(2-amino-3-bromoquinolin-7-yl)ethyl)-5-(4-amino-7H-pyrrolo[2,3-d]pyrimidin-7-yl)-2,3-dimethyltetrahydrofuran-3,4-diol,
(2R,3R,3aS,6S,6aR)-6-((2-amino-3-bromoquinolin-7-yl)oxy)-2-(4-chloro-7H-pyrrolo[2,3-d]pyrimidin-7-yl)hexahydro-2H-cyclopenta[b]furan-3,3a-diol,
(2R,3R,3aS,6S,6aR)-6-((2-amino-3-bromoquinolin-7-yl)oxy)-2-(4-amino-7H-pyrrolo[2,3-d]pyrimidin-7-yl)hexahydro-2H-cyclopenta[b]furan-3,3a-diol,
(2R,3R,3aS,6S,6aR)-6-((2-amino-3-bromoquinolin-7-yl)oxy)-2-(4-methyl-7H-pyrrolo[2,3-d]pyrimidin-7-yl)hexahydro-2H-cyclopenta[b]furan-3,3a-diol,
(2R,3R,3aS,6S,6aR)-6-((2-amino-3-bromoquinolin-7-yl)methyl)-2-(4-methyl-7H-pyrrolo[2,3-d]pyrimidin-7-yl)hexahydro-2H-cyclopenta[b]furan-3,3a-diol,
(2R,3R,3aS,6S,6aR)-6-((2-amino-3-bromoquinolin-7-yl)methyl)-2-(4-amino-7H-pyrrolo[2,3-d]pyrimidin-7-yl)hexahydro-2H-cyclopenta[b]furan-3,3a-diol,
(2R,3S,4R,5R)-2-(((2-amino-3-bromoquinolin-7-yl)oxy)methyl)-5-(4-amino-7H-pyrrolo[2,3-d]pyrimidin-7-yl)-3-methyltetrahydrofuran-3,4-diol,
(2R,3S,4R,5R)-2-{[(2-amino-3-bromoquinolin-7-yl)oxy]methyl}-5-(4-chloro-7H-pyrrolo[2,3-d]pyrimidin-7-yl)-3-methyltetrahydrofuran-3,4-diol,
(2R,3S,4R,5R)-2-(((2-amino-3-bromoquinolin-7-yl)oxy)methyl)-5-(4-methoxy-7H-pyrrolo[2,3-d]pyrimidin-7-yl)-2-methyltetrahydrofuran-3,4-diol,
(2R,3S,4R,5R)-2-(((2-amino-3-bromoquinolin-7-yl)oxy)methyl)-5-(4-amino-7H-pyrrolo[2,3-d]pyrimidin-7-yl)-2-methyltetrahydrofuran-3,4-diol,
(2R,3S,4R,5R)-2-(((2-amino-3-bromoquinolin-7-yl)oxy)methyl)-5-(4-amino-7H-pyrrolo[2,3-d]pyrimidin-7-yl)-3-methyltetrahydrofuran-3,4-diol,
(1S,2R,3S,5R)-3-[2-(2-amino-3-bromo-7-quinolinyl)ethyl]-5-(4-amino-7H-pyrrolo[2,3-d]pyrimidin-7-yl)-3-methyl-1,2-cyclopentanediol,
(1S,2R,3aR,4S,6aR)-4-((2-amino-3-fluoroquinolin-7-yl)methyl)-2-(4-methyl-7H-pyrrolo[2,3-d]pyrimidin-7-yl)hexahydropentalene-1,6a(1H)-diol,
(1S,2R,3aR,4S,6aR)-4-((2-amino-3-chloroquinolin-7-yl)methyl)-2-(4-methyl-7H-pyrrolo[2,3-d]pyrimidin-7-yl)hexahydropentalene-1,6a(1H)-diol,
(1S,2R,3aR,4R,6aR)-4-((2-amino-3-fluoroquinolin-7-yl)methyl)-2-(4-methyl-7H-pyrrolo[2,3-d]pyrimidin-7-yl)hexahydropentalene-1,6a(1H)-diol,
(1S,2R,3aR,4S,6aR)-4-((2-amino-3-bromoquinolin-7-yl)methyl)-2-(4-methyl-7H-pyrrolo[2,3-d]pyrimidin-7-yl)hexahydropentalene-1,6a(1H)-diol,
(2R,3R,3aS,6S,6aR)-6-[(2-amino-3,8-difluoroquinolin-7-yl)methyl]-2-(4-amino-7H-pyrrolo[2,3-d]pyrimidin-7-yl)hexahydro-3aH-cyclopenta[b]furan-3,3a-diol,
(2R,3R,3aS,6S,6aR)-6-[(2-amino-3-chloro-5-fluoroquinolin-7-yl)methyl]-2-(4-amino-7H-pyrrolo[2,3-d]pyrimidin-7-yl)hexahydro-3aH-cyclopenta[b]furan-3,3a-diol,
(2R,3R,3aS,6S,6aR)-6-[(2-amino-3-chloro-8-fluoroquinolin-7-yl)methyl]-2-(4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-(difluoromethyl)quinolin-7-yl)methyl)-2-(4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3,5-difluoroquinolin-7-yl)methyl)-2-(4-amino-5-fluoro-7H-pyrrolo[2,3-d]pyrimidin-7-yl)hexahydro-3aH-cyclopenta[b]furan-3,3a-diol,
(2R,3R,3aS,6S,6aR)-6-((6-amino-7-fluoro-1,5-naphthyridin-3-yl)methyl)-2-(4-methyl-7H-pyrrolo[2,3-d]pyrimidin-7-yl)hexahydro-3aH-cyclopenta[b]furan-3,3a-diol, 3HCl,
(2R,3R,3aS,6S,6aR)-6-((2-amino-3-chloro-8-fluoroquinolin-7-yl)methyl)-2-(4-amino-7H-pyrrolo[2,3-d]pyrimidin-7-yl)hexahydro-3aH-cyclopenta[b]furan-3,3a-diol,
(2R,3R,3aS,6S,6aR)-6-[(2-amino-3,6-difluoroquinolin-7-yl)methyl]-2-(4-amino-7H-pyrrolo[2,3-d]pyrimidin-7-yl)hexahydro-3aH-cyclopenta[b]furan-3,3a-diol,
(2R,3R,3aS,6S,6aR)-6-((7-amino-6-chloro-1,8-naphthyridin-2-yl)methyl)-2-(4-amino-7H-pyrrolo[2,3-d]pyrimidin-7-yl)hexahydro-3aH-cyclopenta[b]furan-3,3a-diol trihydrochloride,
(2R,3R,3aS,6S,6aR)-6-[(2-amino-3,5-difluoroquinolin-7-yl)methyl]-2-(4-amino-7H-pyrrolo[2,3-d]pyrimidin-7-yl)hexahydro-3aH-cyclopenta[b]furan-3,3a-diol,
(1S,2R,3aR,4S,6aR)-4-((2-amino-3-fluoroquinolin-7-yl)methyl)-2-(4-methyl-7H-pyrrolo[2,3-d]pyrimidin-7-yl)hexahydropentalene-1,6a(1H)-diol,
(1S,2R,3aR,4S,6aR)-2-(4-amino-2-methyl-7H-pyrrolo[2,3-d]pyrimidin-7-yl)-4-((2-amino-3-chloroquinolin-7-yl)methyl)hexahydropentalene-1,6a(1H)-diol 2,2,2-trifluoroacetate,
(1S,2R,3aR,4S,6aR)-2-(4-amino-2-methyl-7H-pyrrolo[2,3-d]pyrimidin-7-yl)-4-((2-amino-3-fluoroquinolin-7-yl)methyl)hexahydropentalene-1,6a(1H)-diol 2,2,2-trifluoroacetate,
(1S,2R,3aR,4S,6aR)-4-((2-amino-3-fluoroquinolin-7-yl)methyl)-2-(4-amino-5-fluoro-7H-pyrrolo[2,3-d]pyrimidin-7-yl)hexahydropentalene-1,6a(1H)-diol,
(1S,2R,3aR,4S,6aR)-4-((2-amino-3-chloroquinolin-7-yl)methyl)-2-(4-amino-5-fluoro-7H-pyrrolo[2,3-d]pyrimidin-7-yl)hexahydropentalene-1,6a(1H)-diol,
(1S,2R,3aR,4S,6aR)-4-[(2-amino-3,5-difluoroquinolin-7-yl)methyl]-2-(4-amino-5-methyl-7H-pyrrolo[2,3-d]pyrimidin-7-yl)hexahydropentalene-1,6a(1H)-diol,
(1S,2R,3aR,4S,6aR)-4-[(2-amino-3-chloro-5-fluoroquinolin-7-yl)methyl]-2-(4-amino-5-methyl-7H-pyrrolo[2,3-d]pyrimidin-7-yl)hexahydropentalene-1,6a(1H)-diol,
(1S,2R,3aR,4S,6aR)-2-(4-amino-2-methyl-7H-pyrrolo[2,3-d]pyrimidin-7-yl)-4-((2-amino-3-bromoquinolin-7-yl)methyl)hexahydropentalene-1,6a(1H)-diol,
(1S,2R,3aR,4S,6aR)-4-((2-amino-3-fluoroquinolin-7-yl)methyl)-2-(4-amino-5-methyl-7H-pyrrolo[2,3-d]pyrimidin-7-yl)hexahydropentalene-1,6a(1H)-diol,
(1S,2R,3aR,4S,6aR)-4-((2-amino-3-chloroquinolin-7-yl)methyl)-2-(4-amino-5-methyl-7H-pyrrolo[2,3-d]pyrimidin-7-yl)hexahydropentalene-1,6a(1H)-diol,
(1S,2R,3aR,4S,6aR)-4-((2-amino-3-bromoquinolin-7-yl)methyl)-2-(4-amino-5-methyl-7H-pyrrolo[2,3-d]pyrimidin-7-yl)hexahydropentalene-1,6a(1H)-diol,
(1S,2R,3aR,4S,6aR)-4-((2-amino-3-bromoquinolin-7-yl)methyl)-2-(4-amino-7H-pyrrolo[2,3-d]pyrimidin-7-yl)hexahydropentalene-1,6a(1H)-diol 2,2,2-trifluoroacetate,
(1S,2R,3aR,4S,6aR)-4-((2-amino-3-bromoquinolin-7-yl)methyl)-2-(4-amino-5-fluoro-7H-pyrrolo[2,3-d]pyrimidin-7-yl)hexahydropentalene-1,6a(1H)-diol,
(2R,3R,3aS,6S,6aR)-6-[(2-amino-3-bromoquinolin-7-yl)methyl]-2-[4-amino-5-(difluoromethyl)-7H-pyrrolo[2,3-d]pyrimidin-7-yl]hexahydro-3aH-cyclopenta[b]furan-3,3a-diol,
(2R,3R,3aS,6S,6aR)-6-((2-amino-3-fluoroquinolin-7-yl)methyl)-2-(4-amino-5-fluoro-2-methyl-7H-pyrrolo[2,3-d]pyrimidin-7-yl)hexahydro-3aH-cyclopenta[b]furan-3,3a-diol 2,2,2-trifluoroacetate,
(2R,3R,3aS,6S,6aR)-2-(4-amino-2-fluoro-7H-pyrrolo[2,3-d]pyrimidin-7-yl)-6-((2-amino-3-fluoroquinolin-7-yl)methyl)hexahydro-3aH-cyclopenta[b]furan-3,3a-diol,
(2R,3R,3aS,6S,6aR)-6-((2-amino-3-bromoquinolin-7-yl)oxy)-2-(2-amino-7H-pyrrolo[2,3-d]pyrimidin-7-yl)hexahydro-3aH-cyclopenta[b]furan-3,3a-diol,
(2R,3R,3aS,6S,6aR)-2-(4-amino-2-chloro-7H-pyrrolo[2,3-d]pyrimidin-7-yl)-6-[(2-amino-3-chloroquinolin-7-yl)methyl]hexahydro-3aH-cyclopenta[b]furan-3,3a-diol,
(2R,3R,3aS,6S,6aR)-2-(4-amino-2-chloro-7H-pyrrolo[2,3-d]pyrimidin-7-yl)-6-[(2-amino-3-fluoroquinolin-7-yl)methyl]hexahydro-3aH-cyclopenta[b]furan-3,3a-diol,
(2R,3R,3aS,6S,6aR)-6-[(2-amino-3-bromoquinolin-7-yl)oxy]-2-(4-amino-5-phenyl-7H-pyrrolo[2,3-d]pyrimidin-7-yl)hexahydro-3aH-cyclopenta[b]furan-3,3a-diol,
(2R,3R,3aS,6S,6aR)-2-(4-amino-5-cyclopropyl-7H-pyrrolo[2,3-d]pyrimidin-7-yl)-6-[(2-amino-3-fluoroquinolin-7-yl)methyl]hexahydro-3aH-cyclopenta[b]furan-3,3a-diol,
(2R,3R,3aS,6S,6aR)-2-[4-amino-5-(difluoromethyl)-7H-pyrrolo[2,3-d]pyrimidin-7-yl]-6-[(2-amino-3-fluoroquinolin-7-yl)methyl]hexahydro-3aH-cyclopenta[b]furan-3,3a-diol,
(2R,3R,3aS,6S,6aR)-6-[(2-amino-3-bromoquinolin-7-yl)oxy]-2-(4-amino-5-cyclopropyl-7H-pyrrolo[2,3-d]pyrimidin-7-yl)hexahydro-3aH-cyclopenta[b]furan-3,3a-diol,
(2R,3R,3aS,6S,6aR)-6-[(2-amino-3-fluoroquinolin-7-yl)methyl]-2-(4-amino-7H-pyrrolo[2,3-d]pyrimidin-7-yl)-5,5-difluorohexahydro-3aH-cyclopenta[b]furan-3,3a-diol,
(2R,3R,3aS,5S,6S,6aR)-6-[(2-amino-3-fluoroquinolin-7-yl)methyl]-2-(4-amino-7H-pyrrolo[2,3-d]pyrimidin-7-yl)-5-fluorohexahydro-3aH-cyclopenta[b]furan-3,3a-diol,
(2R,3R,3aS,5S,6S,6aR)-2-(4-amino-5-fluoro-7H-pyrrolo[2,3-d]pyrimidin-7-yl)-6-[(2-amino-3-fluoroquinolin-7-yl)methyl]-5-fluorohexahydro-3aH-cyclopenta[b]furan-3,3a-diol, 2,2,2-trifluoroethanol,
(2R,3R,3aS,6S,6aR)-6-((2-amino-3-bromoquinolin-7-yl)oxy)-2-(4-amino-5-fluoro-7H-pyrrolo[2,3-d]pyrimidin-7-yl)hexahydro-3aH-cyclopenta[b]furan-3,3a-diol,
(2R,3R,3aS,6S,6aR)-2-(4-amino-7H-pyrrolo[2,3-d]pyrimidin-7-yl)-6-((2-((2,2,2-trifluoroethyl)amino)quinolin-7-yl)oxy)hexahydro-3aH-cyclopenta[b]furan-3,3a-diol,
(2R,3R,3aS,6S,6aR)-2-(4-amino-7H-pyrrolo[2,3-d]pyrimidin-7-yl)-6-((2-((cyclopropylmethyl)amino)quinolin-7-yl)oxy)hexahydro-3aH-cyclopenta[b]furan-3,3a-diol,
(2R,3R,3aS,6S,6aR)-6-((2-amino-3-fluoroquinolin-7-yl)methyl)-2-(4-amino-5-fluoro-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)oxy)-2-(4-amino-5-methyl-7H-pyrrolo[2,3-d]pyrimidin-7-yl)hexahydro-3aH-cyclopenta[b]furan-3,3a-diol,
(2R,3R,3aS,6S,6aR)-2-(4-amino-2-methyl-7H-pyrrolo[2,3-d]pyrimidin-7-yl)-6-((2-amino-3-bromoquinolin-7-yl)methyl)hexahydro-3aH-cyclopenta[b]furan-3,3a-diol,
(2R,3R,3aS,6S,6aR)-6-((2-amino-3-fluoroquinolin-7-yl)methyl)-2-(4-amino-5-methyl-7H-pyrrolo[2,3-d]pyrimidin-7-yl)hexahydro-3aH-cyclopenta[b]furan-3,3a-diol 2,2,2-trifluoroacetate,
(2R,3R,3aS,6S,6aR)-2-(4-amino-2-methyl-7H-pyrrolo[2,3-d]pyrimidin-7-yl)-6-((2-amino-3-bromoquinolin-7-yl)oxy)hexahydro-3aH-cyclopenta[b]furan-3,3a-diol,
(2R,3R,3aS,6S,6aR)-6-((2-amino-3-chloroquinolin-7-yl)oxy)-2-(4-amino-5-methyl-7H-pyrrolo[2,3-d]pyrimidin-7-yl)hexahydro-3aH-cyclopenta[b]furan-3,3a-diol 2,2,2-trifluoroacetate,
(2R,3R,3aS,6S,6aR)-6-((2-amino-3-fluoroquinolin-7-yl)oxy)-2-(4-amino-5-methyl-7H-pyrrolo[2,3-d]pyrimidin-7-yl)hexahydro-3aH-cyclopenta[b]furan-3,3a-diol 2,2,2-trifluoroacetate,
(2R,3R,3aS,6S,6aR)-6-((2-amino-3-chloroquinolin-7-yl)methyl)-2-(4-amino-5-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-chloroquinolin-7-yl)methyl)-2-(4-amino-7H-pyrrolo[2,3-d]pyrimidin-7-yl)hexahydro-3aH-cyclopenta[b]furan-3,3a-diol,
(2R,3R,3aS,6S,6aR)-6-((2-amino-3-chloroquinolin-7-yl)methyl)-2-(4-amino-5-fluoro-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)oxy)-2-(4-amino-5-ethyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)methyl)-2-(4-amino-5-methyl-7H-pyrrolo[2,3-d]pyrimidin-7-yl)hexahydro-3aH-cyclopenta[b]furan-3,3a-diol 2,2,2-trifluoroacetate,
(2R,3R,3aS,6S,6aR)-6-((2-amino-3-fluoroquinolin-7-yl)oxy)-2-(4-amino-7H-pyrrolo[2,3-d]pyrimidin-7-yl)hexahydro-3aH-cyclopenta[b]furan-3,3a-diol,
(2R,3R,3aS,6S,6aR)-6-((2-amino-3-chloroquinolin-7-yl)oxy)-2-(4-amino-7H-pyrrolo[2,3-d]pyrimidin-7-yl)hexahydro-3aH-cyclopenta[b]furan-3,3a-diol,
(2R,3R,3aS,6S,6aR)-6-((2-amino-3-fluoroquinolin-7-yl)oxy)-2-(4-amino-5-fluoro-7H-pyrrolo[2,3-d]pyrimidin-7-yl)hexahydro-3aH-cyclopenta[b]furan-3,3a-diol,
(2R,3R,3aS,6S,6aR)-6-((2-amino-3-chloroquinolin-7-yl)oxy)-2-(4-amino-5-fluoro-7H-pyrrolo[2,3-d]pyrimidin-7-yl)hexahydro-3aH-cyclopenta[b]furan-3,3a-diol,
(2R,3R,3aS,6S,6aR)-2-(4-amino-7H-pyrrolo[2,3-d]pyrimidin-7-yl)-6-((2,3-dihydro-1H-pyrrolo[2,3-b]quinolin-7-yl)oxy)hexahydro-3aH-cyclopenta[b]furan-3,3a-diol,
(2R,3R,3aS,6S,6aR)-6-((2-amino-3-(trifluoromethyl)quinolin-7-yl)methyl)-2-(4-amino-7H-pyrrolo[2,3-d]pyrimidin-7-yl)hexahydro-3aH-cyclopenta[b]furan-3,3a-diol,
(2R,3R,3aS,6S,6aR)-6-[(2-amino-3-bromoquinolin-7-yl)oxy]-2-[4-(hydroxymethyl)-7H-pyrrolo[2,3-d]pyrimidin-7-yl]hexahydro-3aH-cyclopenta[b]furan-3,3a-diol,
(2R,3R,3aS,6S,6aR)-6-[(2-amino-3-bromoquinolin-7-yl)oxy]-2-[4-(2-hydroxypropan-2-yl)-7H-pyrrolo[2,3-d]pyrimidin-7-yl]hexahydro-3aH-cyclopenta[b]furan-3,3a-diol,
(2R,3R,3aS,6S,6aR)-6-((2-amino-3-bromoquinolin-7-yl)oxy)-2-(4-(difluoromethyl)-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)oxy)-2-(2,4-dimethyl-7H-pyrrolo[2,3-d]pyrimidin-7-yl)hexahydro-3aH-cyclopenta[b]furan-3,3a-diol 2,2,2-trifluoroacetate,
(2R,3R,3aS,6S,6aR)-6-((2-amino-3-bromoquinolin-7-yl)methyl)-2-(2,4-dimethyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)oxy)-2-(4-amino-5-ethyl-7H-pyrrolo[2,3-d]pyrimidin-7-yl)hexahydro-3aH-cyclopenta[b]furan-3,3a-diol 2,2,2-trifluoroacetate,
(2R,3R,3aS,6S,6aR)-6-((2-amino-3-bromoquinolin-7-yl)methyl)-2-(4-isopropyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)methyl)-2-(5-fluoro-4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)oxy)-2-(7H-pyrrolo[2,3-d]pyrimidin-7-yl)hexahydro-3aH-cyclopenta[b]furan-3,3a-diol,
(2R,3R,3aS,6S,6aR)-6-((2-amino-3-fluoroquinolin-7-yl)oxy)-2-(4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)methyl)-2-(4-cyclopropyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-(trifluoromethyl)quinolin-7-yl)methyl)-2-(4-methyl-7H-pyrrolo[2,3-d]pyrimidin-7-yl)hexahydro-3aH-cyclopenta[b]furan-3,3a-diol,
(2R,3R,3aS,6S,6aR)-6-((2,3-dihydro-1H-pyrrolo[2,3-b]quinolin-7-yl)oxy)-2-(4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-fluoroquinolin-7-yl)methyl)-2-(2,4-dimethyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-chloroquinolin-7-yl)methyl)-2-(2,4-dimethyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)methyl)-2-(4-(methylamino)-7H-pyrrolo[2,3-d]pyrimidin-7-yl)hexahydro-3aH-cyclopenta[b]furan-3,3a-diol,
(1R,2S,3R,5R)-5-(((2-amino-3-bromoquinolin-7-yl)oxy)methyl)-1-methyl-3-(4-(methylamino)-7H-pyrrolo[2,3-d]pyrimidin-7-yl)cyclopentane-1,2-diol,
(2R,3R,3aS,6S,6aR)-6-((2-amino-3-bromoquinolin-7-yl)oxy)-2-(4-(methylamino)-7H-pyrrolo[2,3-d]pyrimidin-7-yl)hexahydro-3aH-cyclopenta[b]furan-3,3a-diol,
(2R,3R,3aS,6S,6aR)-6-((2-amino-3-fluoroquinolin-7-yl)methyl)-2-(4-(methylamino)-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)methyl)-2-(5-fluoro-4-(methylamino)-7H-pyrrolo[2,3-d]pyrimidin-7-yl)hexahydro-3aH-cyclopenta[b]furan-3,3a-diol,
(1S,2R,3aR,4S,6aR)-4-((2-amino-3-bromoquinolin-7-yl)methyl)-2-(4-(methylamino)-7H-pyrrolo[2,3-d]pyrimidin-7-yl)hexahydropentalene-1,6a(1H)-diol,
(2R,3S,4R,5R)-5-(4-amino-7H-pyrrolo[2,3-d]pyrimidin-7-yl)-2-methyl-2-((quinolin-7-yloxy)methyl)tetrahydrofuran-3,4-diol,
(2R,3S,4R,5R)-5-(4-amino-7H-pyrrolo[2,3-d]pyrimidin-7-yl)-3-methyl-2-((quinolin-7-yloxy)methyl)tetrahydrofuran-3,4-diol,
(1S,2R,3R,5R)-3-(((2-amino-3-bromoquinolin-7-yl)oxy)methyl)-5-(4-amino-7H-pyrrolo[2,3-d]pyrimidin-7-yl)-3-methylcyclopentane-1,2-diol,
(1S,2R,3R,5R)-5-(4-amino-7H-pyrrolo[2,3-d]pyrimidin-7-yl)-3-(((2-aminoquinolin-7-yl)oxy)methyl)-3-methylcyclopentane-1,2-diol,
(1S,2R,5R)-3-(2-(2-amino-3-bromoquinolin-7-yl)ethyl)-5-(4-amino-7H-pyrrolo[2,3-d]pyrimidin-7-yl)-3-(fluoromethyl)cyclopentane-1,2-diol,
(1R,2S,3R,5S)-5-(2-(2-amino-3-bromoquinolin-7-yl)ethyl)-3-(4-amino-7H-pyrrolo[2,3-d]pyrimidin-7-yl)-1-methylcyclopentane-1,2-diol,
(2R,3R,3aS,6S,6aR)-2-(4-amino-7H-pyrrolo[2,3-d]pyrimidin-7-yl)-6-((2-aminoquinolin-7-yl)oxy)hexahydro-2H-cyclopenta[b]furan-3,3a-diol,
(1R,2S,3R,5R)-5-(((2-aminoquinolin-7-yl)oxy)methyl)-1-methyl-3-(7H-pyrrolo[2,3-d]pyrimidin-7-yl)cyclopentane-1,2-diol,
(1R,2S,3R,5R)-5-(((2-amino-3-methylquinolin-7-yl)oxy)methyl)-1-methyl-3-(4-methyl-7H-pyrrolo[2,3-d]pyrimidin-7-yl)cyclopentane-1,2-diol,
(2R,3R,3aS,6S,6aR)-6-((2-aminoquinolin-7-yl)methyl)-2-(4-methyl-7H-pyrrolo[2,3-d]pyrimidin-7-yl)hexahydro-2H-cyclopenta[b]furan-3,3a-diol,
(2R,3R,3aS,6S,6aR)-6-((2-amino-3-bromoquinolin-7-yl)methyl)-2-(4-amino-5-fluoro-7H-pyrrolo[2,3-d]pyrimidin-7-yl)hexahydro-2H-cyclopenta[b]furan-3,3a-diol,
(1S,2R,3S,5R)-3-(2-(2-amino-3-bromoquinolin-7-yl)ethyl)-3-methyl-5-(4-methyl-7H-pyrrolo[2,3-d]pyrimidin-7-yl)cyclopentane-1,2-diol,
(1S,2R,3S,5R)-3-(2-(2,3-dihydro-1H-pyrrolo[2,3-b]quinolin-7-yl)ethyl)-3-methyl-5-(4-methyl-7H-pyrrolo[2,3-d]pyrimidin-7-yl)cyclopentane-1,2-diol,
(1R,2S,3R,5R)-5-(((2-amino-3-bromoquinolin-7-yl)oxy)methyl)-3-(4-amino-7H-pyrrolo[2,3-d]pyrimidin-7-yl)-1,5-dimethylcyclopentane-1,2-diol,
(2R,3R,3aS,6S,6aR)-6-((2-amino-3-chloroquinolin-7-yl)oxy)-2-(4-methyl-7H-pyrrolo[2,3-d]pyrimidin-7-yl)hexahydro-2H-cyclopenta[b]furan-3,3a-diol,
(2R,3R,3aS,6S,6aR)-6-((2-aminoquinolin-7-yl)oxy)-2-(4-methyl-7H-pyrrolo[2,3-d]pyrimidin-7-yl)hexahydro-2H-cyclopenta[b]furan-3,3a-diol,
(2R,3R,3aS,6S,6aR)-6-((2-amino-3-bromoquinolin-7-yl)oxy)-2-(2-amino-4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-(difluoromethyl)quinolin-7-yl)oxy)-2-(4-methyl-7H-pyrrolo[2,3-d]pyrimidin-7-yl)hexahydro-3aH-cyclopenta[b]furan-3,3a-diol,
(1S,2R,3R,5R)-3-(2-(2-amino-3-fluoroquinolin-7-yl)ethyl)-5-(4-amino-7H-pyrrolo[2,3-d]pyrimidin-7-yl)-3-methylcyclopentane-1,2-diol,
(1R,2S,3R,5S)-5-(2-(2-amino-3-bromoquinolin-7-yl)ethyl)-3-(4-amino-7H-pyrrolo[2,3-d]pyrimidin-7-yl)-1,5-dimethylcyclopentane-1,2-diol,
(1R,2S,3S,4R)-1-(2-(2-amino-3-bromoquinolin-7-yl)ethyl)-4-(4-amino-7H-pyrrolo[2,3-d]pyrimidin-7-yl)-2-methylcyclopentane-1,2,3-triol,
(1S,2R,3aR,4S,6aR)-4-((2-amino-3-bromoquinolin-7-yl)oxy)-2-(4-amino-7H-pyrrolo[2,3-d]pyrimidin-7-yl)hexahydropentalene-1,6a(1H)-diol 2,2,2-trifluoroacetate,
(2R,3R,3aS,6S,6aR)-6-((2-amino-3-fluoroquinolin-7-yl)amino)-2-(4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-fluoroquinolin-7-yl)methyl)-2-(4-amino-7H-pyrrolo[2,3-d]pyrimidin-7-yl)-6a-methylhexahydro-3aH-cyclopenta[b]furan-3,3a-diol,
(2R,3R,3aS,6S,6aR)-6-((2-amino-3-chloroquinolin-7-yl)methyl)-2-(4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-fluoroquinolin-7-yl)methyl)-2-(4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-fluoroquinolin-7-yl)methyl)-2-(4-amino-7H-pyrrolo[2,3-d]pyrimidin-7-yl)hexahydro-3aH-cyclopenta[b]furan-3,3a-diol,
(2R,3R,3aS,6S,6aR)-6-((2-amino-3-methylquinolin-7-yl)methyl)-2-(4-amino-7H-pyrrolo[2,3-d]pyrimidin-7-yl)hexahydro-3aH-cyclopenta[b]furan-3,3a-diol,
(1S,2R,3S,5R)-5-(4-amino-7H-pyrrolo[2,3-d]pyrimidin-7-yl)-3-(2-(2,3-dihydro-1H-pyrrolo[2,3-b]quinolin-7-yl)ethyl)-3-methylcyclopentane-1,2-diol,
(2R,3R,3aS,6S,6aR)-6-((2-amino-3-bromoquinolin-7-yl)methyl)-2-(2-amino-4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-(difluoromethyl)quinolin-7-yl)oxy)-2-(4-amino-7H-pyrrolo[2,3-d]pyrimidin-7-yl)hexahydro-3aH-cyclopenta[b]furan-3,3a-diol,
(2R,3R,3aS,6R,6aR)-6-((2-amino-3-bromoquinolin-7-yl)oxy)-2-(4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)oxy)-2-(2-amino-5-fluoro-7H-pyrrolo[2,3-d]pyrimidin-7-yl)hexahydro-3aH-cyclopenta[b]furan-3,3a-diol,
(2R,3R,3aS,6R,6aR)-6-((2-amino-3-chloroquinolin-7-yl)methyl)-2-(4-methyl-7H-pyrrolo[2,3-d]pyrimidin-7-yl)hexahydro-3aH-cyclopenta[b]furan-3,3a-diol,
(3aS,4S,5R)-1-((2-amino-3-bromoquinolin-7-yl)methyl)-5-(4-amino-7H-pyrrolo[2,3-d]pyrimidin-7-yl)tetrahydro-1H-cyclopenta[c]furan-3a,4(3H)-diol,
(2R,3R,3aS,6S,6aR)-6-((2-amino-3-bromoquinolin-7-yl)methyl)-2-(4-methyl-7H-pyrrolo[2,3-d]pyrimidin-7-yl)tetrahydrofuro[3,4-b]furan-3,3a(4H)-diol,
(2R,3R,3aS,6R,6aR)-6-((2-amino-3-bromoquinolin-7-yl)methyl)-2-(4-methyl-7H-pyrrolo[2,3-d]pyrimidin-7-yl)tetrahydrofuro[3,4-b]furan-3,3a(4H)-diol,
(2R,3R,3aS,6S,6aR)-6-((2-amino-3-bromoquinolin-7-yl)methyl)-2-(4-amino-5-methyl-7H-pyrrolo[2,3-d]pyrimidin-7-yl)tetrahydrofuro[3,4-b]furan-3,3a(4H)-diol,
(2R,3R,3aS,6S,6aR)-6-((2-amino-3-bromoquinolin-7-yl)methyl)-2-(4-amino-7H-pyrrolo[2,3-d]pyrimidin-7-yl)tetrahydrofuro[3,4-b]furan-3,3a(4H)-diol, or
(2R,3R,3aS,6S,6aR)-6-((2-amino-3-(difluoromethyl)quinolin-7-yl)methyl)-2-(4-amino-7H-pyrrolo[2,3-d]pyrimidin-7-yl)hexahydro-2H-cyclopenta[b]furan-3,3a-diol,
(4-(3-Oxa-8-azabicyclo[3.2.1]octane-8-carbonyl)-2-ethoxy-N—((R)-2-hydroxy-2-((S)-1,2,3,4-tetrahydroisoquinolin-3-yl)ethyl)benzamide),
   or a pharmaceutically acceptable salt thereof.

Disclosed herein are methods of treating cancer in a patient comprising: evaluating a biological sample from the patient for the presence of one or more mutations or alteration in one of the following genes: FLT3, NPM1, DNMT3a, SRSF2, ZRSR2 or SF3B1, and treating the patient with a PRMT5 inhibitor if one or more mutations including FLT3, NPM1, DNMT3a, SRSF2, ZRSR2 or SF3B1 are present in the sample.

In a subembodiment, the evaluating step comprises: isolating DNA from a biological sample; and sequencing the DNA to determine the presence of any mutation or alteration in any one of the following genes: FLT3, NPM1, DNMT3a, SRSF2, ZRSR2 or SF3B1.

In a subembodiment, the samples would be submitted for testing on a tNGS panel. In a further subembodiment, the testing would be performed on a TruSight™, a Myeloid Sequencing Panel or a Foundation One Heme Panel.

Isolating DNA from the biological sample can be performed by a number of procedures known to one skilled in the art. In one embodiment, DNA can be isolated from the biological sample using an AllPrep FFPE Kit from Qiagen (Product Number: 80234) or QIAamp DNA Blood Mini Kit from Qiagen (Product Number: 51104).

The methods described herein are generally applicable to determining the expression levels of biomarkers and can be used to identify a patient who is likely to be responsive to PRMT5 inhibitor.

The present invention includes kits and primers for identifying the presence of one or more mutation or alterations as described above in a biological sample.

Biomarkers or biomarker gene expression may be detected using commercially available kits, or using custom assays with commercially available anti-biomarker antibodies obtained from suppliers well known in the art, or using custom assays and antibodies raised by the investigator.

The disclosed methods, kits, and primers may be understood more readily by reference to the following detailed description taken in connection with the accompanying figures, which form a part of this disclosure. It is to be understood that the disclosed methods, kits, and primers are not limited to the specific methods, kits, and primers described and/or shown herein, and that the terminology used herein is for the purpose of describing particular embodiments by way of example only and is not intended to be limiting of the claimed methods, kits, and primers.

Reference to numerical value includes at least that particular value, unless the context clearly dictates otherwise. When a range of values is expressed, the range includes from the one particular value and/or to the other particular value. Further, reference to values stated in ranges include each and every value within that range. All ranges are inclusive and combinable.

It is to be appreciated that certain features of the disclosed methods, kits, and primers which are, for clarity, described herein in the context of separate embodiments, may also be provided in combination in a single embodiment. Conversely, various features of the disclosed methods, kits, and primers that are, for brevity, described in the context of a single embodiment, may also be provided separately or in any subcombination.

Disclosed herein are methods of identifying a patient and treating a patient who will have a high likelihood to be responsive to treatment with a protein arginine N-methyltransferase 5 (PRMT5) inhibitor.

PRMT5 inhibitors may bind to the PRMT5 enzyme, competitively or cooperatively with natural substrate SAM (S-adenosyl-L-methionine), to inhibit such enzyme.

The methods provided herein are useful for the treatment of cancer through identification of a biomarker. Cancers that may be treated include, but are not limited to: (1) Cardiac: sarcoma (angiosarcoma, fibrosarcoma, rhabdomyosarcoma, liposarcoma), myxoma, rhabdomyoma, fibroma, lipoma and teratoma; (2) Lung: bronchogenic carcinoma (squamous cell, undifferentiated small cell, undifferentiated large cell, adenocarcinoma), alveolar (bronchiolar) carcinoma, bronchial adenoma, sarcoma, lymphoma, chondromatous hamartoma, mesothelioma, non-small cell; (3) Gastrointestinal: esophagus (squamous cell carcinoma, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel (adenocarcinoma, lymphoma, carcinoid tumors, Karposi's sarcoma, leiomyoma, hemangioma, lipoma, neurofibroma, fibroma), large bowel (adenocarcinoma, tubular adenoma, villous adenoma, hamartoma, leiomyoma), colon, colorectal, rectal; (4) Genitourinary tract: kidney (adenocarcinoma, Wilm's tumor [nephroblastoma], lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); (5) Liver: hepatoma (hepatocellular carcinoma), cholangiocarcinoma, hepatoblastoma, angiosarcoma, hepatocellular adenoma, hemangioma; (6) Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; (7) Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma [pinealoma], glioblastoma multiform, oligodendroglioma, schwannoma, retinoblastoma, congenital tumors), spinal cord neurofibroma, meningioma, glioma, sarcoma); (8) Gynecological: uterus (endometrial carcinoma), cervix (cervical carcinoma, pre-tumor cervical dysplasia), ovaries (ovarian carcinoma [serous cystadenocarcinoma, mucinous cystadenocarcinoma, unclassified carcinoma], granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; (9) Hematologic: blood (myeloid leukemia [acute and chronic], acute lymphoblastic leukemia, chronic lymphocytic leukemia, chronic myelomonocytic (CMML), myeloproliferative diseases, multiple myeloma, myelodysplastic syndrome), Hodgkin's disease, non-Hodgkin's lymphoma [malignant lymphoma]; (10) Skin: malignant melanoma, basal cell carcinoma, squamous cell carcinoma, Karposi's sarcoma, moles dysplastic nevi, lipoma, angioma, dermatofibroma, keloids, psoriasis; and (11) Adrenal glands: neuroblastoma. Examples of cancer that may be treated by the compounds, compositions and methods of the invention include thyroid cancer, anaplastic thyroid carcinoma, epidermal cancer, head and neck cancer (e.g., squamous cell cancer of the head and neck), sarcoma, tetracarcinoma, hepatoma and multiple myeloma. Thus, the term “cancerous cell” as provided herein, includes a cell afflicted by any one of the above-identified conditions.

In one example of the invention the cancer treated is colo-rectal cancer (such as, for example, colon adenocarcinoma and colon adenoma). In one example of the invention the cancer treated is melanoma.

Examples of cancers which may be treated, include, but are not limited to: colo-rectal cancer (such as, for example, colon adenocarcinoma and colon adenoma). Examples of cancers which may be treated, include, but are not limited to: melanoma.

Examples of blood disorders which may be treated, include, but are not limited to, hemoglobinopathy, such as sickle cell disease or β-thalassemia.

As used herein, the singular forms “a,” “an,” and “the” include the plural.

“Biomarker” is an objectively measured indicator that reflects the presence, process, event, condition, progression, or successful treatment of a particular condition. The terms “biomarker” or “marker” are used interchangeably herein. A biomarker is a nucleic acid or polypeptide and the presence (positivity) or absence (negativity) of a mutation or differential expression of the polypeptide is used to determine sensitivity to any PRMT5 inhibitor.

As used herein, “treating” and like terms refer to reducing the severity and/or frequency of cancer symptoms, eliminating cancer symptoms and/or the underlying cause of said symptoms, reducing the frequency or likelihood of cancer symptoms and/or their underlying cause, and improving or remediating damage caused, directly or indirectly, by cancer.

“Biological samples” refers to any sample from a patient in which cancerous cells can be obtained and RNA can be isolated. Suitable biological samples include, but are not limited to, blood, lymph fluid, bone marrow, a solid tumor sample, or any combination thereof.

“Next-generation sequencing” or “NGS” refers to any sequencing method that determines the nucleotide sequence of either individual nucleic acid molecules (e.g., in single molecule sequencing) or clonally expanded proxies for individual nucleic acid molecules in a high throughput parallel fashion (e.g., greater than 103, 104, 105 or more molecules can be sequenced simultaneously). Exemplary next generation sequencing techniques include sequencing by synthesis, sequencing by ligation, sequencing by hybridization. Exemplary next generations sequencing methods include TruSight™ Myeloid Sequencing Panel. A key advantage that NGS holds over single-gene assays is the ability to assess up to thousands of genes in a single experiment. NGS provides the efficiency of library preparation in a single tube with a large number of targets. Potentially eliminating sequential testing by covering all possible targets in the first round may be a valuable feature for researchers trying to conserve limited samples. Descriptions of certain NGS platforms can be found in the following: Shendure, et al, “Next-generation DNA sequencing,” Nature, 2008, vol. 26, No. 10, 1135-1145.

### Next Generation Sequencing Platforms

TruSight™ Myeloid Sequencing Panel is a proven next-generation sequencing technology to identify somatic mutations in hematologic malignancies. The TruSight Myeloid Sequencing Panel uses NGS technology to provide a comprehensive assessment of 54 genes (tumor suppressor genes and oncogenic hotspots) in one assay.

### PRMT5 Inhibitors for Use in the Disclosed Methods

Suitable PRMT5 inhibitors for use in the disclosed methods are provided herein. A PRMT5 inhibitor refers to any compound capable of inhibiting the production, level, activity, expression or presence of PRMT5. In some embodiments, if one or more mutations or alterations are present in the sample, including FLT3 internal tandem duplication (ITD), NPM1 mutation, DNMT3A mutation, SRSF2, SF3B1, ZRSR2 mutation or alteration, the patient can be treated with a PRMT5 inhibitor. PRMT5 inhibitors disclosed in U.S. application Ser. Nos. 15/508,053, 62/583,250, 62/583,258, 62/715,324, 62/715,447, 62/715,446, 14/769,402, 15/508,074, 15/508,053, 15/508,063, 16/081,696, 16/081,745, 16/082,230, 16/082,256, 16/082,196, 16/082,219, 62/672,451, or similar applications (incorporated herein by reference), including any tautomeric or stereochemically isomeric form thereof, and a N-oxide thereof, a pharmaceutically acceptable salts thereof, or a solvate thereof. In some aspects, for example, the patient can be treated with Example 138 as found in the present application, including any tautomeric or sterochemically isomeric form thereof, and N-oxide thereof, a pharmaceutically acceptable salts thereof or a solvate thereof. In some aspects, the pharmaceutically acceptable salt is a HCl salt.

In some embodiments the patient can be treated with a PRMT5 inhibitor if one or more mutations or amplifications including FLT3 internal tandem duplication (ITD), NPM1 mutation, DNMT3A mutation, SRSF2, SF3B1, ZRSR2 mutation or alteration are present in the sample, wherein the PRMT5 inhibitor is an anti-PRMT5 antibody.

Salts can be synthesized from a parent compound that contains a basic or acidic moiety by conventional chemical methods such as methods described in Pharmaceutical Salts: Properties, Selection, and use, P. Heinrich Stahl (Editor), Camille G. Wermuth (Edutor), ISBNL 3-90639-026-8, Hardcover, 388 pages, August 2002, which is incorporated herein by reference. Generally, such salts can be prepared by reacting the free acid or base forms of these compounds with the appropriate base or acid in water or in an organic solvent, or in a mixture of the two generally, non-aqueous media such as ether, ethyl acetate, ethanol, isopropanol, or acetonitrile are used. The PRMT5 inhibitors for use in the disclosed methods may exist as mono- or di- salts depending upon the pKa of the acid from which the salt is formed.

As used herein, the terms “measuring expression levels,” “measuring gene expression level,” or “obtaining an expression level” and the like, includes methods that quantify target gene expression level exemplified by a transcript of a gene, including microRNA (miRNA) or a protein encoded by a gene, as well as methods that determine whether a gene of interest is expressed at all. Thus, an assay which provides a “yes” or “no” result without necessarily providing quantification of an amount of expression is an assay that “measures expression” as that term is used herein. Alternatively, the term may include quantifying expression level of the target gene expressed in a quantitative value, for example, a fold-change in expression, up or down, relative to a control gene or relative to the same gene in another sample

As used herein, “subject” refers to an organism or to a cell sample, tissue sample or organ sample derived therefrom, including, for example, cultured cell lines, biopsy, blood sample or fluid sample containing a cell. In many instances, the subject or sample derived there from, comprises a plurality of cell types. In one embodiment, the sample includes, for example, a mixture of tumor cells and normal cells. In one embodiment, the sample comprises at least 10%, 15%, 20%, et seq., 90%, or 95% tumor cells. In one embodiment, the organism is a mammal, such as, a human, canine, murine, feline, bovine, ovine, swine, or caprine. In a particular embodiment, the organism is a human patient.

“Patient” as that term is used herein, refers to the recipient in need of medical intervention or treatment. Mammalian and non-mammalian patients are included. In one embodiment, the patient is a mammal, such as, a human, canine, murine, feline, bovine, ovine, swine, or caprine. In a particular embodiment, the patient is a human.

The term “treating” in its various grammatical forms in relation to the present invention refers to preventing (i.e. chemoprevention), curing, reversing, attenuating, alleviating, minimizing, suppressing or halting the deleterious effects of a disease state, disease progression, disease causative agent (e.g., bacteria or viruses) or other abnormal condition. For example, treatment may involve alleviating a symptom (i.e., not necessary all symptoms) of a disease or attenuating the progression of a disease.

“Treatment of cancer”, as used herein, refers to partially or totally inhibiting, delaying or preventing the progression of cancer including cancer metastasis; inhibiting, delaying or preventing the recurrence of cancer including cancer metastasis; or preventing the onset or development of cancer (chemoprevention) in a mammal, for example a human. In addition, the methods of the present invention may be practiced for the treatment of chemoprevention of human patients with cancer. However, it is also likely that the methods would also be effective in the treatment of cancer in other mammals.

As used herein, the term “therapeutically effective amount” is intended to qualify the amount of the treatment in a therapeutic regimen necessary to treat cancer. This includes combination therapy involving the use of multiple therapeutic agents, such as a combined amount of a first and second treatment where the combined amount will achieve the desired biological response. The desired biological response is partial or total inhibition, delay or prevention of the progression of cancer including cancer metastasis; inhibition, delay or prevention of the recurrence of cancer including cancer metastasis; or the prevention of the onset or development of cancer (chemoprevention) in a mammal, for example a human.

As used herein, the term “solvate” means a physical association of the compound with one or more solvent molecules. This physical association involves varying degrees of ionic and covalent bonding, including hydrogen bonding. In certain instances, the solvate will be capable of isolation, for example, when one or more solvent molecules are incorporated in the crystal lattice of the crystalline solid. The term “solvate” is intended to encompass both solution-phase and isolatable solvates. Non-limiting examples of suitable solvates include the disclosed compounds in combination with water, isopropanol, ethanol, methanol, DMSO, ethyl acetate, acetic acid or ethanolamine and the like.

Solvates are well known in the pharmaceutical chemistry. They can be important to the processes for the preparation of a substance (e.g. in relation to their purification), the storage of the substance (e.g. its stability) and the ease of handling of the substance and are often formed as part of the isolation or purification stages of a chemical synthesis. A person skilled in the art can determine by means of standard and long used techniques whether a hydrate or other solvate has formed by the isolation conditions or purification conditions used to prepare a given compound. Examples of such techniques include thermogravimetric analysis (TGA), differential scanning calorimetry (DSC), X-ray crystallography (e.g. single crystal X-ray crystallography or X-ray powder diffraction) and Solid-State NMR (SS-NMR, also known as magic Angle Spinning NMR or MAS-NMR). Such techniques are part of the standard analytical toolkit of the skilled chemist as NMR, IR, HPLC, and MS. Alternatively, the skilled person can deliberately form a solvate using crystallization conditions that include an amount of the solvent required for the particular solvate. Thereafter, the standard methods described above, can be used to establish whether solvates had formed.

The term “composition” is intended to encompass a product comprising the specified ingredients in the specified amounts, as well as any product which results, directly or indirectly, from combination of the specified ingredients in the specified amounts. The term “anti-cancer agent” means a drug (medicament or pharmaceutically active ingredient) for treating cancer. The term “antineoplastic agent” means a drug (medicament or pharmaceutically active ingredient) for treating cancer (i.e., a chemotherapeutic agent). The term “at least one” means one or more than one. The meaning of “at least one” with reference to the number of disclosed compounds is independent of the meaning with reference to the number of chemotherapeutic agents. The term “chemotherapeutic agent” means a drug (medicament or pharmaceutically active ingredient) for treating cancer (i.e., an antineoplastic agent). The term “compound” with reference to the antineoplastic agents, includes the agents that are antibodies. The term “consecutively” means one following the other. The term “effective amount” means a “therapeutically effective amount”. The term “therapeutically effective amount” means that amount of active compound or pharmaceutical agent that elicits the biological or medicinal response in a tissue, system, animal or human that is being sought by a researcher, veterinarian, medical doctor or other clinician. Thus, for example, in the methods of treating cancer described herein “effective amount” (or “therapeutically effective amount”) means, the amount of the compound (or drug), or radiation, that results in: (a) the reduction, alleviation or disappearance of one or more symptoms caused by the cancer, (b) the reduction of tumor size, (c) the elimination of the tumor, and/or (d) long-term disease stabilization (growth arrest) of the tumor. Also, for example, an effective amount, or a therapeutically effective amount of the PRMT5 inhibitor (i.e., PRMT5 inhibitor to be administered to the patient may be administered) is that amount which results in the reduction in PRMT5 activity. The term “treating cancer” or “treatment of cancer” refers to administration to a mammal afflicted with a cancerous condition and refers to an effect that alleviates the cancerous condition by killing the cancerous cells and refers to an effect that results in the inhibition of growth and/or metastasis of the cancer.

“Combination” refers to either a fixed combination in one dosage unit form, or a combined administration where a compound of the present invention and a combination partner (e.g. another drug as explained below, also referred to as “therapeutic agent” or “co-agent”) may be administered independently at the same time or separately within time intervals, especially where these time intervals allow that the combination partners show a cooperative, e.g. synergistic effect. The single components may be packaged in a kit or separately. One or both components (e.g., powders or liquids) may be reconstituted or diluted to a desired dose prior to administration.

The terms “co-administration” or “combined administration” or the like as utilized herein are meant to encompass administration of the selected combination partner to a single subject in need thereof (e.g. a patient) and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients.

The term “fixed combination” means that the active ingredients, e.g. PRMT5 inhibitor to be administered to the patient in the present invention and a combination partner, are both administered to a subject simultaneously in the form of a single entity or dosage.

The term “non-fixed combination” means that the active ingredients, e.g. PRMT5 inhibitor to be administered to the patient in the present invention and a combination partner, are both administered to a subject as separate entities either simultaneously, concurrently or sequentially with no specific time limits, wherein such administration provides therapeutically effective levels of the two compounds in the body of the subject. The latter also applies to cocktail therapy, e.g. the administration of three or more active ingredients.

In any of the methods of identifying a patient predicted to be responsive to a treatment with PRMT5 inhibitor, described herein, unless stated otherwise, the methods can optionally include the administration of an effective amount of radiation therapy. For radiation therapy, γ-radiation is preferred. Methods for the safe and effective administration of most of these chemotherapeutic agents are known to those skilled in the art. In addition, their administration is described in the standard literature. For example, the administration of many of the chemotherapeutic agents is described in the “Physicians' Desk Reference” (PDR), e.g., 64^{th} Edition, 2010 (published by PDR Network, LLC at Montvale, N.J. 07645-1725), presently accessible through www.pdr.net; the disclosures of which are incorporated herein by reference thereto.

If the patient is responding, or is stable, after completion of the therapy cycle, the therapy cycle can be repeated according to the judgment of the skilled clinician. Upon completion of the therapy cycles, the patient can be continued on a PRMT5 inhibitor, as an example, but not limited to, one of the disclosed compounds at the same dose that was administered in the treatment protocol. This maintenance dose can be continued until the patient progresses or can no longer tolerate the dose (in which case the dose can be reduced, and the patient can be continued on the reduced dose).

Those skilled in the art will recognize that the actual dosages and protocols for administration employed in the methods of the invention may be varied according to the judgment of the skilled clinician. The actual dosage employed may be varied depending upon the requirements of the patient and the severity of the condition being treated. Determination of the proper dosage for a particular situation is within the skill of the art. A determination to vary the dosages and protocols for administration may be made after the skilled clinician considers such factors as the patient's age, condition and size, as well as the severity of the cancer being treated and the response of the patient to the treatment.

The amount and frequency of administration of the PRMT5 inhibitor and additionally the optional chemotherapeutic agents will be regulated according to the judgment of the attending clinician (physician) considering such factors as age, condition and size of the patient as well as severity of the cancer being treated.

The PRMT5 inhibitor can be administered according to therapeutic protocols well known in the art. It will be apparent to those skilled in the art that the administration of the chemotherapeutic agent can be varied depending on the cancer being treated and the known effects of the chemotherapeutic agent on that disease. Also, in accordance with the knowledge of the skilled clinician, the therapeutic protocols (e.g., dosage amounts and times of administration) can be varied in view of the observed effects of the administered therapeutic agents on the patient, and in view of the observed responses of the cancer to the administered therapeutic agents.

The determination of the order of administration of the PRMT5 inhibitor once the patient is identified, and the number of repetitions of administration of the chemotherapeutic agent during a treatment protocol, is well within the knowledge of the skilled physician after evaluation of the cancer being treated and the condition of the patient.

Thus, in accordance with experience and knowledge, the practicing physician can modify each protocol for the administration of a chemotherapeutic agent according to the individual patient's needs, as the treatment proceeds. All such modifications are within the scope of the present invention.

The attending clinician, in judging whether treatment is effective at the dosage administered, will consider the general well-being of the patient as well as more definite signs such as relief of cancer-related symptoms (e.g., pain), inhibition of tumor growth, actual shrinkage of the tumor, or inhibition of metastasis. Size of the tumor can be measured by standard methods such as radiological studies, e.g., CAT or MRI scan, and successive measurements can be used to judge whether growth of the tumor has been retarded or even reversed. Relief of disease-related symptoms such as pain, and improvement in overall condition can also be used to help judge effectiveness of treatment.

Another example of the instant invention is the method of identifying a patient predicted to be responsive to a treatment with a PRMT5 inhibitor and treatment of such patient with a PRMT5 inhibitor in combination with gene therapy for the treatment of cancer. For an overview of genetic strategies to treating cancer see Hall et al., (Am. J. Hum. Genet. 61:785-789, 1997) and Kufe et al., (Cancer Medicine, 5th Ed, pp 876-889, BC Decker, Hamilton 2000). Gene therapy can be used to deliver any tumor suppressing gene. Examples of such genes include, but are not limited to, p53, which can be delivered via recombinant virus-mediated gene transfer (see U.S. Pat. No. 6,069,134, for example), a uPA/uPAR antagonist (“Adenovirus-Mediated Delivery of a uPA/uPAR Antagonist Suppresses Angiogenesis-Dependent Tumor Growth and Dissemination in Mice,” Gene Therapy, August 1998; 5(8):1105-13), and interferon gamma (J. Immunol. 2000; 164:217-222).

It is understood that substituents and substitution patterns on the disclosed compounds can be selected by one of ordinary skill in the art to provide compounds that are chemically stable and that can be readily synthesized by techniques known in the art, as well as those methods set forth below, from readily available starting materials. If a substituent is itself substituted with more than one group, it is understood that these multiple groups may be on the same carbon or on different carbons, so long as a stable structure results. Also, “optionally substituted” means either unsubstituted or substituted with the specified groups, radicals or moieties.

The present invention includes a method of identifying a patient diagnosed with cancer predicted to be responsible to a treatment with a PRMT5 inhibitor, not limited to but for example, one of the disclosed compounds listed in the present application, as well as the pharmaceutically acceptable salts thereof, and salts that are not pharmaceutically acceptable when they are used as precursors to the free compounds or their pharmaceutically acceptable salts or in other synthetic manipulations.

The PRMT5 inhibitor to be administered to the patient may be administered in the form of a pharmaceutically acceptable salt. The term “pharmaceutically acceptable salt” refers to salts prepared from pharmaceutically acceptable non-toxic bases or acids including inorganic or organic bases and inorganic or organic acids. Salts of basic compounds encompassed within the term “pharmaceutically acceptable salt” refer to non-toxic salts of the disclosed compounds which are generally prepared by reacting the free base with a suitable organic or inorganic acid. Representative salts of basic compounds of the present invention include, but are not limited to, the following: acetate, ascorbate, adipate, alginate, aspirate, benzenesulfonate, benzoate, bicarbonate, bisulfate, bitartrate, borate, bromide, 4-bromobenzenesulfonate, butyrate, camphorate, camphorsulfonate, camsylate, carbonate, chloride, clavulanate, citrate, cyclohexylamidosulfonate, cyclopentane propionate, diethylacetic, digluconate, dihydrochloride, dodecylsulfanate, edetate, edisylate, estolate, esylate, ethanesulfonate, formic, fumarate, gluceptate, glucoheptanoate, gluconate, glucuonate, glutamate, glycerophosphate, glycollylarsanilate, hemisulfate, heptanoate, hexanoate, hexylresorcinate, hydrabamine, hydrobromide, hydrochloride, 2-hydroxyethanesulfonate, hydroxynaphthoate, iodide, isonicotinic, isothionate, lactate, lactobionate, laurate, malate, maleate, mandelate, mesylate, methylbromide, methylnitrate, methylsulfate, methanesulfonate, mucate, 2-naphthalenesulfonate, napsylate, nicotinate, nitrate, N-methylglucamine ammonium salt, oleate, oxalate, pamoate (embonate), palmitate, pantothenate, pectinate, persulfate, phosphate/diphosphate, pimelic, phenylpropionic, polygalacturonate, propionate, salicylate, stearate, sulfate, subacetate, succinate, tannate, tartrate, teoclate, thiocyanate, tosylate, triethiodide, trifluoroacetate, trifluoromethylsulfonate, p-toluenesulfonate, undeconate, valerate and the like.

Furthermore, where the disclosed compounds carry an acidic moiety, suitable pharmaceutically acceptable salts thereof include, but are not limited to, salts derived from inorganic bases including aluminum, ammonium, calcium, copper, ferric, ferrous, lithium, magnesium, manganic, mangamous, potassium, sodium, zinc, and the like. Particularly preferred are the ammonium, calcium, magnesium, potassium, and sodium salts.

With basic reagents such as hydroxides, carbonates, hydrogencarbonates, alkoxides and ammonia, organic bases or alternatively basic amino acids the compounds form stable alkali metal, alkaline earth metal or optionally substituted ammonium salts. Salts derived from pharmaceutically acceptable organic non-toxic bases include salts of primary, secondary, and tertiary amines, cyclic amines, dicyclohexyl amines and basic ion-exchange resins, such as arginine, betaine, caffeine, choline, N,N-dibenzylethylenediamine, diethanolamine, diethylamine, 2-diethylaminoethanol, 2-dimethylaminoethanol, ethanolamine, ethylamine, ethylenediamine, N-ethylmorpholine, N-ethylpiperidine, glucamine, glucosamine, histidine, hydrabamine, isopropylamine, lysine, methylglucamine, morpholine, ornithine, piperazine, piperidine, polyamine resins, procaine, purines, theobromine, triethanolamine, triethylamine, trimethylamine, tripropylamine, trometamol, tromethamine, and the like. Also, included are the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl; and diamyl sulfates, long chain halides such as decyl, lauryl, myristyl and stearyl chlorides, bromides and iodides, aralkyl halides like benzyl and phenethyl bromides and others.

The preparation of pharmacologically acceptable salts from one of the disclosed compounds capable of salt formation, including their stereoisomeric forms is carried out known methods, for example, by mixing a disclosed compound with an equivalent amount of a solution containing a desired acid, base, or the like, and then collecting the desired salt by filtering the salt or distilling off the solvent. The compounds of the present invention and salts thereof may form solvates with a solvent such as water, ethanol, or glycerol. The compounds of the present invention may form an acid addition salt and a salt with a base at the same time according to the type of substituent of the side chain.

The present invention encompasses treatment of a patient with a PRMT5 inhibitor disclosed in this application and all stereoisomeric forms of the disclosed compounds. When bonds to a chiral carbon are depicted as straight lines in the structural formulas of the invention, it is understood that both the (R) and (S) configurations of the chiral carbon, and hence both enantiomers and mixtures thereof, are embraced within the disclosed compounds. Similarly, when a compound name is recited without a chiral designation for a chiral carbon, it is understood that both the (R) and (S) configurations of the chiral carbon, and hence individual enantiomers and mixtures thereof, are embraced by the name. The production of specific stereoisomers or mixtures thereof may be identified in the Examples where such stereoisomers or mixtures were obtained.

The disclosed compounds include all possible enantiomers and diastereomers and mixtures of two or more stereoisomers, for example mixtures of enantiomers and/or diastereomers, in all ratios. Thus, enantiomers are a subject of the invention in enantiomerically pure form, both as levorotatory and as dextrorotatory antipodes, in the form of racemates and in the form of mixtures of the two enantiomers in all ratios. In the case of a cis/trans isomerism the invention includes both the cis form and the trans form as well as mixtures of these forms in all ratios. The preparation of individual stereoisomers can be carried out, if desired, by separation of a mixture by customary methods, for example by chromatography or crystallization, by the use of stereochemically uniform starting materials for the synthesis or by stereoselective synthesis. Optionally a derivatization can be carried out before a separation of stereoisomers. The separation of a mixture of stereoisomers can be carried out at an intermediate step during the synthesis of a disclosed compounds, or it can be done on a final racemic product. Absolute stereochemistry may be determined by X-ray crystallography of crystalline products or crystalline intermediates which are derivatized, if necessary, with a reagent containing a stereogenic center of known configuration. Where disclosed compounds are capable of tautomerization, all individual tautomers as well as mixtures thereof are included in the scope of the invention. The present invention includes all such isomers, as well as salts, solvates (including hydrates) and solvated salts of such racemates, enantiomers, diastereomers and tautomers and mixtures thereof.

In the disclosed compounds, the atoms may exhibit their natural isotopic abundances, or one or more of the atoms may be artificially enriched in a particular isotope having the same atomic number, but an atomic mass or mass number different from the atomic mass or mass number predominantly found in nature. The present invention is meant to include all suitable isotopic variations of the specifically and generically described compounds. For example, different isotopic forms of hydrogen (H) include protium (¹H) and deuterium (²H). Protium is the predominant hydrogen isotope found in nature. Enriching for deuterium may afford certain therapeutic advantages, such as increasing in vivo half-life or reducing dosage requirements, or may provide a compound useful as a standard for characterization of biological samples. Isotopically-enriched compounds can be prepared without undue experimentation by conventional techniques well known to those skilled in the art or by processes analogous to those described in the general process schemes and examples herein using appropriate isotopically-enriched reagents and/or intermediates.

Furthermore, the disclosed compounds may exist in amorphous form and/or one or more crystalline forms, and as such all amorphous and crystalline forms and mixtures thereof of the disclosed compounds are intended to be included within the scope of the present invention. In addition, some of the compounds of the instant invention may form solvates with water (i.e., a hydrate) or common organic solvents. Such solvates and hydrates, particularly the pharmaceutically acceptable solvates and hydrates, of the instant compounds are likewise encompassed within the scope of the invention, along with un-solvated and anhydrous forms.

If the disclosed compounds simultaneously contain acidic and basic groups in the molecule the invention also includes, in addition to the salt forms mentioned, inner salts or betaines (zwitterions). Salts can be obtained from the disclosed compounds by customary methods which are known to the person skilled in the art, for example by combination with an organic or inorganic acid or base in a solvent or dispersant, or by anion exchange or cation exchange from other salts. The present invention also includes all salts of the disclosed compounds which, owing to low physiological compatibility, are not directly suitable for use in pharmaceuticals but which can be used, for example, as intermediates for chemical reactions or for the preparation of physiologically acceptable salts.

The PRMT5 compounds useful in the present methods, include derivatives of the disclosed compounds acting as prodrugs and solvates. Prodrugs, following administration to the patient, are converted in the body by normal metabolic or chemical processes, such as through hydrolysis in the blood, to the disclosed compounds.

The treatment with a PRMT5 inhibitor according to the invention can be administered by oral, inhalative, rectal or transdermal administration or by subcutaneous, intraarticular, intraperitoneal or intravenous injection. Oral administration is preferred. Coating of stents with disclosed compounds and other surfaces which come into contact with blood in the body is possible.

Suitable solid or galenical preparation forms are, for example, granules, powders, coated tablets, tablets, (micro)capsules, suppositories, syrups, juices, suspensions, emulsions, drops or injectable solutions and preparations having prolonged release of active substance, in whose preparation customary excipients such as vehicles, disintegrants, binders, coating agents, swelling agents, glidants or lubricants, flavorings, sweeteners and solubilizers are used. Frequently used auxiliaries which may be mentioned are magnesium carbonate, titanium dioxide, lactose, mannitol and other sugars, talc, lactose, gelatin, starch, cellulose and its derivatives, animal and plant oils such as cod liver oil, sunflower, peanut or sesame oil, polyethylene glycol and solvents such as, for example, sterile water and mono- or polyhydric alcohols such as glycerol.

The dosage regimen utilizing the compounds is selected in accordance with a variety of factors including type, species, age, weight, sex and medical condition of the patient; the severity of the condition to be treated; the route of administration; the renal and hepatic function of the patient; and the particular compound or salt thereof employed. An ordinarily skilled physician or veterinarian can readily determine and prescribe the effective amount of the drug required to prevent, counter, or arrest the progress of the condition.

Oral dosages of the compounds, when used for the indicated effects, will range between about 0.01 mg per kg of body weight per day (mg/kg/day) to about 30 mg/kg/day, preferably 0.025-7.5 mg/kg/day, more preferably 0.1-2.5 mg/kg/day, and most preferably 0.1-0.5 mg/kg/day (unless specified otherwise, amounts of active ingredients are on free base basis). For example, an 80 kg patient would receive between about 0.8 mg/day and 2.4 g/day, preferably 2-600 mg/day, more preferably 8-200 mg/day, and most preferably 8-40 mg/kg/day. A suitably prepared medicament for once a day administration would thus contain between 0.8 mg and 2.4 g, preferably between 2 mg and 600 mg, more preferably between 8 mg and 200 mg, and most preferably 8 mg and 40 mg, e.g., 8 mg, 10 mg, 20 mg and 40 mg. Advantageously, the compounds may be administered in divided doses of two, three, or four times daily. For administration twice a day, a suitably prepared medicament would contain between 0.4 mg and 4 g, preferably between 1 mg and 300 mg, more preferably between 4 mg and 100 mg, and most preferably 4 mg and 20 mg, e.g., 4 mg, 5 mg, 10 mg and 20 mg.

Intravenously, the patient would receive the active ingredient in quantities sufficient to deliver about 0.01 mg per kg of body weight per day (mg/kg/day) to about 30 mg/kg/day, preferably 0.025-7.5 mg/kg/day, more preferably 0.1-2.5 mg/kg/day, and even more preferably 0.1-0.5 mg/kg/day. Such quantities may be administered in a number of suitable ways, e.g. large volumes of low concentrations of active ingredient during one extended period of time or several times a day, low volumes of high concentrations of active ingredient during a short period of time, e.g. once a day. Typically, a conventional intravenous formulation may be prepared which contains a concentration of active ingredient of between about 0.01-1.0 mg/ml, e.g. 0.1 mg/ml, 0.3 mg/ml, and 0.6 mg/ml, and administered in amounts per day of between 0.01 ml/kg patient weight and 10.0 ml/kg patient weight, e.g. 0.1 ml/kg, 0.2 ml/kg, 0.5 ml/kg. In one example, an 80 kg patient, receiving 8 ml twice a day of an intravenous formulation having a concentration of active ingredient of 0.5 mg/ml, receives 8 mg of active ingredient per day. Glucuronic acid, L-lactic acid, acetic acid, citric acid or any pharmaceutically acceptable acid/conjugate base with reasonable buffering capacity in the pH range acceptable for intravenous administration may be used as buffers. The choice of appropriate buffer and pH of a formulation, depending on solubility of the drug to be administered, is readily made by a person having ordinary skill in the art.

“Celite®” (Fluka) diatomite is diatomaceous earth, and can be referred to as “Celite”.

The disclosed compounds may be prepared by employing reactions as shown in the following Reaction Schemes, in addition to other standard manipulations that are known in the literature or exemplified in the experimental procedures. The illustrative Reaction Schemes below, therefore, are not limited by the compounds listed or by any particular substituents employed for illustrative purposes. Substituent numbering as shown in the Reaction Schemes do not necessarily correlate to that used in the claims and often, for clarity, a single substituent is shown attached to the compound where multiple substituents are optionally allowed under the disclosed compounds hereinabove.

Methods for Identifying Biomarkers in Patients that Will be Responsive to Treatment with a PRMT5 inhibitor.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

The disclosures of each patent, patent application, and publication cited or described in this document are hereby incorporated herein by reference, in its entirety.

### Methods for Making the Disclosed PRMT5 Compounds

### General Methods

The PRMT5 compounds useful in the present invention can be readily produced from known compounds or commercially available compounds by, for example, known processes described in published documents, and produced by production processes described below.

It should be noted that, when a disclosed compound has a reactive group such as hydroxy group, amino group, carboxyl group, or thiol group as its substituent, such group may be adequately protected with a protective group in each reaction step and the protective group may be removed at an adequate stage. The process of such introduction and removal of the protective group may be adequately determined depending on the group to be protected and the type of the protective group, and such introduction and removal are conducted, for example, by the process described in the review section of Greene, T. W., et. al., “Protective Groups in Organic Synthesis”, 2007, 4th Ed., Wiley, New York, or Kocienski, P., “Protecting Groups” 1994, Thieme.

It should be noted that, if a discrepancy between the chemical name and structure exists, the structure is understood to dominate.

All solvents used were commercially available and were used without further purification. Reactions were typically run using anhydrous solvents under an inert atmosphere of nitrogen.

Starting materials used were either available from commercial sources or prepared according to literature procedures and had experimental data in accordance with those reported.

Abbreviations used are those conventional in the art of the following:
ACN acetonitrile
AcOH acetic acid
ADDP 1,1′-(azodicarbonyl)dipiperidine
AIBN α,α′-azoisobutyronitrile
AML acute myeloid leukemia
AIMDM Iscove's Modified Dulbecco's Medium for Agar cultures
Ar aryl
Atm atmosphere
aq. aqueous
BBN Borabicyclo(3.3.1)nonane
9-BBN 9-Borabicyclo(3.3.1)nonane
BFU-E primitive erythroid progenitor cells
Bn Benzyl
BnBr Benzyl bromide
BSA bovine serum albumin
Boc Butyloxycarbonyl
Bz benzoyl
° C. degree Celsius
CDCl₃ deuterated chloroform
CD₃OD deuterated methanol
CO carbon monoxide
Cs₂CO₃ cesium carbonate
CuBrMe₂S Copper bromide dimethyl sulfide
CDI carbonyldiimidazole
CDCl₃ deuterated chloroform
conc. concentration
DEA diethylamine
DBU 1,8-diazabicyclo[5.4.0]undec-7-ene
DCA dichloroacetic acid
DCC N—N′-dicyclohexylcarbodiimide
DCE 1,2-dichloroethane
DCM dichloromethane
DDQ 2,3-dichloro-5,6-dicyano-p-benzoquinone
DEAD di-tert-butylazodicarboxylate
DIAD Diisopropyl azodicarboxylate
DIBAL-H diisobutylaluminium hydride
DIEA N,N-diisopropylethylamine
DIPEA N,N-diisopropylethylamine
DMAP 4-(dimethylamino)pyridine
DMF N,N-dimethylformamide
DMP Dess-Martin periodinane
DMSO dimethyl sulfoxide
DMTr 4,4′-dimethoxytrityl
DTT dithiothreitol
dppf 1,1′-bis(diphenylphosphino)ferrocene
EDC N-(3-dimethylaminopropyl)-N′-ethylcarbodiimide
EDCl N-ethyl-N′-(3-dimethylaminopropyl)carbodiimide hydrochloride
EDCl-HCl N-ethyl-N′-(3-dimethylaminopropyl)carbodiimide hydrochloride
EPO Erythropoietin
Et Ethyl
Et₃N triethylamine
EtO Ethoxy
EtOAc ethyl acetate
EtOH ethanol
Equiv (Eq) equivalents (molar)
FBS Fetal Bovine Serum
g gram
G-CSF granulocyte-colony stimulating factor
GM-CSF granulocyte-macrophage colony-stimulating factor
h hour(s)
HATU N-[(Dimethylamino)-1H-1,2,3-triazolo-[4,5-b]pyridin-1-ylmethylene]-N-methylmethanaminium hexafluorophosphate N-oxide HATU 2-(1H-7-azabenzotriazol-1-yl)-1,1,3,3-tetramethyluronium hexafluorophosphate
HCl hydrochloric acid
hM-CSF human macrophage colony stimulating factor
HMPA Hexamethylphosphoramide
HOBt 1-Hydroxybenzotriazole
HPLC high pressure liquid chromatography
IBX 2-iodoxybenzoic acid
Im imidazole
ITD internal tandem duplication
IMDM Iscove's Modified Dulbecco's Medium
iPr isopropyl
K₂CO₃ potassium carbonate
KMnO₄ potassium permanganate
LCMS liquid chromatography and mass spectrometry
LiHMDS lithium bis(trimethylsilyl)amide
LOF Loss of function
M molar
mCPBA me ta-chloroperoxybenzoic acid
Me Methyl
MeO Methoxy
m-CPBA meta-chloroperoxybenzoic acid
MeCN acetonitrile
MeOH methanol
MePPh₃Br Methyltriphenylphosphonium bromide
mg milligram
min minutes
mL milliliter(s)
mmol millimole
MS mass spectrometry
MTBE methyl tert-butyl ether
N normal
ND Not determined
NaBH₄ sodium borohydride
NaH sodium hydride
NaHCO₃ sodium bicarbonate
NaOH sodium hydroxide
NaHSO₄ sodium bisulfate
Na₂SO₄ sodium sulfate
NBS N-bromosuccinimide
NGS Next Generation Sequencing
NH₄HCO₃ ammonium bicarbonate
NH₄Cl ammonium chloride
nM nanomolar
NMO N-methylmorpholine-N-oxide
NMP N-methyl-2-pyrrolidone
nPr n-propyl
NMR nuclear magnetic resonance
OTIPS triisopropylsilyl ether
PBS Phosphate buffered saline
PDC Pyridinium Dichromate
Pd/C palladium on carbon
PdCl₂(dppf) [1,1-bis(diphenylphosphine)ferrocene]dichloropalladium(II)
PdCl₂(PPh₃)₂) trans-dichlorobis(triphenylphosphine)palladium(II)
Pd₂(dba)₃) tris(dibenzylideneacetone) dipalladium(0)
Pd(PPh₃)₄ tetrakis(triphenylphosphine)palladium(0)
Ph Phenyl
Ph₃P Triphenylphosphine
P(n-Bu)₃ Triphenyl phosphine
Prep-TLC preparative TLC
POCl₃ phosphorus(V) oxychloride
Pol polymer-bound
pTsOH p-toluenesulfonic acid
psi pound per square inch
py pyridine
RANKL Receptor activator of nuclear factor kappa-B ligand
Rh(nbd)₂BF₄ Bis(norbornadiene)rhodium(I) tetrafluoroborate
RuPhos Pd G3 (2-Dicyclohexylphosphino-2′,6′-diisopropoxy-1,1′-biphenyl)[2-(2′-amino-1,1′-biphenyl)]palladium(II) methanesulfonate
rt room temperature
sat. saturated
SCF Stem cell factor
SEM 2-(trimethylsilyl)ethoxymethyl
SFC Supercritical fluid chromatography
SM starting material
SOCl₂ thionyl chloride
t-BuOK Potassium t-butoxide
TBAF tetrabutylammonium fluoride
TBDPS tert-butyldiphenylsilyl
TBDPSCl tert-butyl(chloro)diphenylsilane
TBDPSO tert-butyldiphenylsilyl ether
TBHP tert-butyl hydroperoxide
TEA triethylamine
Tf triflyl
TFA trifluoroacetic acid
THF tetrahydrofuran
TIPS Triisopropylsilyl
TLC thin layer chromatography
TMS trimethylsilyl
TMSOTf Trimethylsilyl trifluoromethanesulfonate
TsCl Toluenesulfonyl chloride
TsOH p-toluenesulfonic acid
TPAP Tetrapropylammonium perruthenate
TPO Thrombopoietin
T3P propylphosphonic anhydride
μL microliter
vol volume
xg gravitational force

A panel of primary human acute myeloid leukemia (AML) patient samples were profiled to stratify by growth inhibition by the PRMT5 inhibitor as shown in Compounds 1-138, or a pharmaceutically acceptable salt thereof. Equivalent PRMT5 inhibitors are shown in such compounds as found in U.S. Application No. 62/464,006, PCT/US/19/045050, and U.S. Ser. No. 15/508,053 or European patent application 15757502.8.

### General Synthetic Schemes

In all general schemes, Ar implies either aryl or heteroaryl.

The substituted reagents and starting material were commercially acquired, synthesized as reported above, or synthesized through known routes reported in the literature.

### SYNTHESIS OF INTERMEDIATES

### General Synthetic Schemes Applicable for Examples 1-137

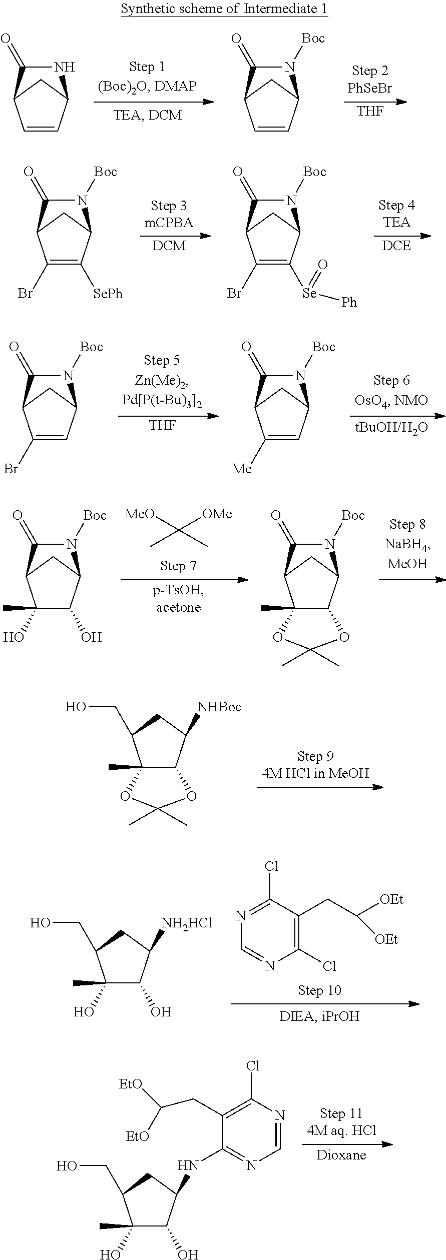

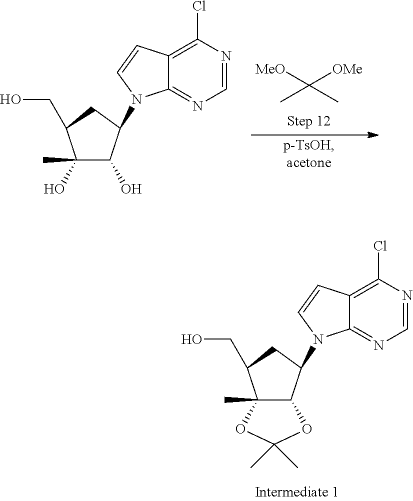

### Intermediate 1: ((3aR,4R,6R,6aS)-6-(4-chloro-7H-pyrrolo[2,3-d]pyrimidin-7-yl)-2,2,3a-trimethyltetrahydro-3aH-cyclopenta[d][1,3]dioxol-4-yl)methanol

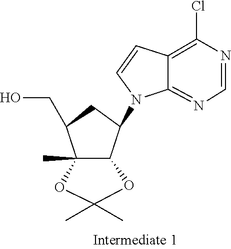 [SMILES: CC1(C)O[C@H]2[C@H](n3ccc4c(Cl)ncnc43)C[C@H](CO)[C@@]2(C)O1]

Step 1: To a mixture of (1R,4S)-2-azabicyclo[2.2.1]hept-5-en-3-one (50 g, 458 mmol), di-tert-butyl dicarbonate (120 g, 550 mmol) and N,N-dimethylpyridin-4-amine (5.6 g, 45.8 mmol) in DCM (500 mL) was added triethylamine (69.5 g, 687 mmol) at 25° C. The reaction mixture was stirred for 2 hours at 25° C. The reaction was quenched by saturated aqueous NaHCO₃ (1500 mL) and extracted with EtOAc (2000 mL×3). The combined organic layers were dried over anhydrous Na₂SO₄ and filtered. The filtrate was concentrated under reduced pressure. The residue was purified by a silica gel column chromatography and eluted with 0-20% of ethyl acetate in petroleum ether to afford (1R,4S)-tert-butyl-3-oxo-2-azabicyclo[2.2.1]hept-5-ene-2-carboxylate. MS: 154 (M−55).

Step 2: To a solution of (1R,4S)-tert-butyl 3-oxo-2-azabicyclo[2.2.1]hept-5-ene-2-carboxylate (40 g, 191 mmol) in THF (400 mL) was added phenyl hypobromoselenoite (49.6 g, 210 mmol) in THF (1.0 L) dropwise at −78° C. under an argon atmosphere. The mixture was stirred for 2 hours at −78° C., and then the temperature was warmed to 25° C. slowly. The reaction mixture was stirred at 25° C. for 16 hours. The reaction was quenched by saturated aqueous NaHCO₃ (500 mL) and extracted with DCM (500 mL×3). The combined organic layers were dried over anhydrous Na₂SO₄ and filtered. The filtrate was concentrated under reduced pressure. The residue was purified by silica gel column chromatography and eluted with 1%-20% of ethyl acetate in petroleum ether to afford (1R,4R)-tert-butyl 5-bromo-3-oxo-6-(phenylselanyl)-2-azabicyclo[2.2.1]heptane-2-carboxylate (mixture of two isomers). MS: 390/392 (M−55/M−53). ¹H NMR (400 MHz, DMSO-d₆) isomer 1: δ 7.73-7.59 (m, 2H), 7.44-7.26 (m, 3H), 4.65 (t, J=4.0 Hz, 1H), 4.38 (s, 1H), 3.55 (t, J=3.4 Hz, 1H), 3.05 (q, J=1.8 Hz, 1H), 2.29-2.16 (m, 1H), 2.04 (dt, J=11.1, 1.4 Hz, 1H), 1.31 (s, 9H). isomer 2: δ 7.73-7.59 (m, 2H), 7.44-7.26 (m, 3H), 4.73 (t, J=2.0 Hz, 1H), 4.33 (dd, J=3.6, 2.0 Hz, 1H), 4.24 (t, J=3.1 Hz, 1H), 3.05 (q, J=1.8 Hz, 1H), 2.42 (dq, J=10.7, 1.9 Hz, 1H), 2.29-2.16 (m, 1H), 1.36 (s, 9H).

Step 3: To a solution of (1R,4R)-tert-butyl 5-bromo-3-oxo-6-(phenylselanyl)-2-azabicyclo[2.2.1]heptane-2-carboxylate (33 g, 74.1 mmol) in DCM (150 mL) was added 3-chloroperbenzoic acid (20.1 g, 82 mmol) in several portions at −78° C. under an argon atmosphere. The resulting mixture was stirred for 2 hours at −78° C. The reaction was quenched by saturated aqueous NaHCO₃ (100 mL) and extracted with DCM (300 mL×3). The combined organic layers were dried over anhydrous Na₂SO₄ and filtered. The filtrate was concentrated under reduced pressure to afford the crude product (1R,4R)-tert-butyl 5-bromo-3-oxo-6-(phenylseleninyl)-2-azabicyclo[2.2.1]heptane-2-carboxylate. MS: 462/464 (M+1/M+3).

Step 4: To a stirred mixture of (1R,4R)-tert-butyl 5-bromo-3-oxo-6-(phenylseleninyl)-2-azabicyclo[2.2.1]heptane-2-carboxylate (127 g, 274 mmol) in DCE (1000 mL) was added triethylamine (76 mL, 549 mmol) at 25° C. The resulting mixture was stirred for 6 hours at 80° C. The reaction was cooled to room temperature and quenched with water (500 mL). The organic layers were separated, washed with brine (100 mL×2), dried over anhydrous sodium sulfate, and filtered. The filtrate was concentrated to dryness. The residue was purified by silica gel column chromatography, eluted with 0-10% of ethyl acetate in petroleum ether to afford (1R,4R)-tert-butyl 5-bromo-3-oxo-2-azabicyclo[2.2.1]hept-5-ene-2-carboxylate. ¹H NMR (400 MHz, DMSO-d₆) δ 7.20 7.20 (d, J=2.6 Hz, 1H), 4.96 (t, J=2.6 Hz, 1H), 3.42 (t, J=2.8 Hz, 1H), 2.40 (t, J=1.8 Hz, 2H), 1.44 (s, 9H).

Step 5 (method A): To a stirred solution of (1R,4R)-tert-butyl 5-bromo-3-oxo-2-azabicyclo[2.2.1]hept-5-ene-2-carboxylate (15 g, 52.1 mmol) in toluene (50 mL) were added Pd(PPh₃)₄ (6.0 g, 5.2 mmol) and tetramethylstannane (28.9 mL, 208 mmol) at 25° C. The mixture was stirred for 6 hours at 100° C. in a sealed tube. The reaction mixture was quenched by saturated NaHCO₃ solution (200 mL) and extracted with EtOAc (300 mL×3). The combined organic layers were dried over anhydrous Na₂SO₄, filtered, and the filtrate was concentrated to dryness. The residue was purified by silica gel column chromatography, eluted with 0-3% EtOAc in a mixture of petroleum ether/DCM (v:v, 5/1) to afford (1R,4S)-tert-butyl 5-methyl-3-oxo-2-azabicyclo[2.2.1]hept-5-ene-2-carboxylate. ¹H NMR (300 MHz, DMSO-d₆) δ 6.51-6.42 (m, 1H), 4.78 (p, J=2.2 Hz, 1H), 3.12 (d, J=2.8 Hz, 1H), 2.25 (d, J=8.4 Hz, 1H), 2.12-2.10 (m, 1H), 1.87 (s, 3H), 1.40 (s, 9H).

Step 5 (method B): To a stirred solution of (1R,4R)-tert-butyl 5-bromo-3-oxo-2-azabicyclo[2.2.1]hept-5-ene-2-carboxylate (26 g, 90 mmol) in THF (250 mL) were added dimethylzinc (1 M in toluene, 180 mL, 180 mmol) dropwise and bis(tri-ter t-butylphosphine)palladium(0) (0.92 g, 1.8 mmol) at 0° C. The resulting mixture was stirred for 16 hours at 20° C. The reaction was quenched by saturated aqueous NH₄Cl (400 mL) and extracted with DCM (500 mL×2). The combined organic layers were dried over anhydrous Na₂SO₄ and filtered. The filtrate was concentrated under reduced pressure. The residue was purified by silica gel column chromatography, eluted with 0-3% EtOAc in a mixture of petroleum ether and DCM (v/v=5:1) to afford (1R,4S)-tert-butyl 5-methyl-3-oxo-2-azabicyclo[2.2.1]hept-5-ene-2-carboxylate. ¹H NMR (400 MHz, DMSO-d₆) δ 6.53-6.47 (m, 1H), 4.82-4.80 (m, 1H), 3.16-3.14 (m, 1H), 2.28 (dt, J=8.4, 1.8 Hz, 1H), 2.12-2.10 (m, 1H), 1.90 (s, 3H), 1.43 (s, 9H).

Step 6: To a solution of (1R,4S)-tert-butyl 5-methyl-3-oxo-2-azabicyclo[2.2.1]hept-5-ene-2-carboxylate (5 g, 22.4 mmol) in tBuOH (25 mL)/water (25 mL) was added 4-methylmorpholine 4-oxide (5.25 g, 44.8 mmol) at 0° C. under argon atmosphere. This was followed by the addition of osmium (VIII) oxide (18.5 mL, 22.4 mmol, 4% in water) dropwise at 0° C. The mixture was stirred for 16 hours at room temperature. The reaction was quenched by the addition of saturated aqueous Na₂S₂O₃ (30 mL), then extracted with EtOAc (30 mL×4). The combined organic layers were washed with brine (30 mL), dried over anhydrous Na₂SO₄ and filtered. The filtrate was concentrated to dryness. The residue was purified by silica gel column chromatography and eluted with 0-70% ethyl acetate in petroleum ether. The fractions containing the desired product were combined and concentrated under reduced pressure to afford (1R,4S,5R,6S)-tert-butyl 5,6-dihydroxy-5-methyl-3-oxo-2-azabicyclo[2.2.1]heptane-2-carboxylate. ¹H NMR (300 MHz, DMSO-d₆) δ 5.54 (d, J=5.7 Hz, 1H), 4.96 (s, 1H), 4.04 (s, 1H), 3.49-3.37 (m, 1H), 2.37 (d, J=2.4 Hz, 1H), 2.11 (dd, J=10.5, 1.8 Hz, 1H), 1.98-1.80 (m, 1H), 1.45 (s, 9H), 1.21 (s, 3H).

Step 7: (1R,4S,5R,6S)-tert-butyl-5,6-dihydroxy-5-methyl-3-oxo-2-azabicyclo[2.2.1]heptane-2-carboxylate (1.4 g, 5.4 mmol) was co-evaporated with dry toluene (10 mL×3) and then re-dissolved in acetone (10 mL). To this solution was added 4-methylbenzenesulfonic acid (0.094 g, 0.5 mmol), followed by the addition of 2,2-dimethoxypropane (2.83 g, 27.2 mmol) at room temperature. The resulting mixture was stirred at ambient temperature for 1 hour. The mixture was neutralized with saturated aqueous NaHCO₃ to pH 7. The mixture was concentrated to dryness. The crude product was purified by silica gel column chromatography and eluted with 10-50% ethyl acetate in petroleum ether to give (3aS,4R,7S,7aR)-tert-butyl 2,2,7a-trimethyl-6-oxotetrahydro-4,7-methano[1,3]dioxolo[4,5-c]pyridine-5(6H)-carboxylate. ¹H NMR (400 MHz, Chloroform-d) δ 4.39 (t, J=1.6 Hz, 1H), 4.21 (d, J=1.5 Hz, 1H), 2.71 (q, J=1.6 Hz, 1H), 2.23-2.19 (m, 1H), 2.07-2.00 (m, 1H), 1.61 (s, 3H), 1.53 (s, 9H), 1.49 (s, 3H), 1.48 (s, 3H).

Step 8: To a solution of (3 aS,4R,7 S,7aR)-tert-butyl 2,2,7a-trimethyl-6-oxotetrahydro-4,7-methano[1,3]dioxolo[4,5-c]pyridine-5(6H)-carboxylate (2.9 g, 9.8 mmol) in MeOH (58 mL) was added NaBH₄ (0.74 g, 19.5 mmol) at 0° C. The mixture was stirred for 2 hours at 0° C. The reaction mixture was quenched by saturated aqueous NH₄Cl (50 mL) and extracted with ethyl acetate (60 mL×3). The combined organic layers were concentrated to dryness. The residue was purified by column chromatography on silica gel and eluted with 0-40% of EtOAc in petroleum ether. The collected fractions were combined and concentrated under vacuum to give tert-butyl ((3aS,4R,6R,6aR)-6-(hydroxymethyl)-2,2,6a-trimethyltetrahydro-3aH-cyclopenta[d][1,3]dioxol-4-yl)carbamate. ¹H NMR (300 MHz, DMSO-d₆) 7.05 (br s, 1H), 4.49 (t, J=5.0 Hz, 1H), 3.86 (d, J=2.8 Hz, 1H), 3.77-3.74 (m, 1H), 3.55-3.44 (m, 1H), 3.31-3.25 (m, 1H), 2.07-1.97 (m, 1H), 2.21-2.14 (m, 1H), 1.40 (s, 9H), 1.40-1.39 (m, 1H), 1.38 (s, 3H), 1.25 (s, 3H), 1.23 (s, 3H).

Step 9: Tert-butyl ((3aS,4R,6R,6aR)-6-(hydroxymethyl)-2,2,6a-trimethyltetrahydro-3aH-cyclopenta[d][1,3]dioxol-4-yl)carbamate (3.5 g, 11.6 mmol) was dissolved in HCl (30 mL, 4M in methanol). The resulting solution was stirred at ambient temperature for 2 h. The solution was concentrated to give the crude product of (1R,2S,3R,5R)-3-amino-5-(hydroxymethyl)-1-methylcyclopentane-1,2-diol hydrochloride. ¹H NMR (300 MHz, DMSO-d₆) δ 8.21 (br s, 3H), 5.21 (br s, 1H), 4.60-4.31 (m, 2H), 3.52 (d, J=9.0 Hz, 1H), 3.44 (dd, J=10.5, 5.1 Hz, 1H), 3.32-3.19 (m, 2H), 2.18-2.07 (m, 1H), 2.00-1.76 (m, 1H), 1.46-1.36 (m, 1H), 1.10 (s, 3H).

Step 10: To a stirred mixture of (1R,2S,3R,5R)-3-amino-5-(hydroxymethyl)-1-methylcyclopentane-1,2-diol hydrochloride (1.85 g, 9.4 mmol) and 4,6-dichloro-5-(2,2-diethoxyethyl)pyrimidine (2.73 g, 10.3 mmol) in 2-propanol (40 mL) was added N-ethyl-N-isopropylpropan-2-amine (2.42 g, 18.7 mmol) at 25° C. The reaction mixture was stirred for 16 hours at 100° C. The reaction mixture was cooled to room temperature and concentrated to dryness. The residue was purified by silica gel column chromatography (0-15% MeOH in DCM) to afford (1R,2S,3R,5R)-3-((6-chloro-5-(2,2-diethoxyethyl)pyrimidin-4-yl)amino)-5-(hydroxymethyl)-1-methylcyclopentane-1,2-diol. MS: 390 (M+1). ¹H NMR (400 MHz, DMSO-d₆) δ 8.15 (s, 1H), 6.79 (d, J=7.5 Hz, 1H), 4.68-4.56 (m, 2H), 4.50-4.40 (m, 1H), 4.37-4.30 (m, 1H), 4.01 (s, 1H), 3.76-3.58 (m, 2H), 3.50-3.39 (m, 4H), 3.35-3.25 (m, 1H), 2.92-2.90 (m, 2H), 2.25-2.18 (m, 1H), 1.94-1.85 (m, 1H), 1.31-1.24 (m, 1H), 1.21-1.02 (m, 9H).

Step 11: To a stirred solution of (1R,2S,3R,5R)-3-((6-chloro-5-(2,2-diethoxyethyl)pyrimidin-4-yl)amino)-5-(hydroxymethyl)-1-methylcyclopentane-1,2-diol (10 g, 25.6 mmol) in 1,4-dioxane (80 mL) was added dropwise aqueous HCl (20 mL, 80 mmol, 4 M in water) at room temperature. The resulting mixture was stirred for 0.5 hours at 50° C. Then the mixture was cooled to 0° C. with an ice bath and neutralized with saturated aqueous NaHCO₃ to pH ˜8 to 9. The resulting mixture was concentrated to dryness and the residue was purified by silica gel column chromatography, eluting with 0-10% of MeOH in DCM to afford (1R,2S,3R,5R)-3-(4-chloro-7H-pyrrolo[2,3-d]pyrimidin-7-yl)-5-(hydroxymethyl)-1-methylcyclopentane-1,2-diol. MS: 298 (M+1). ¹H NMR (400 MHz, DMSO-d₆) δ 8.62 (s, 1H), 7.89 (d, J=3.6 Hz, 1H), 6.71 (d, J=3.6 Hz, 1H), 5.10 (q, J=9.6 Hz, 1H), 4.86 (d, J=7.2 Hz, 1H), 4.73 (t, J=4.8 Hz, 1H), 4.27 (s, 1H), 4.05 (dd, J=9.6, 7.2 Hz, 1H), 3.54-3.51 (m, 2H), 2.41-2.33 (m, 1H), 2.05-1.95 (m, 1H), 1.73-1.66 (m, 1H), 1.21 (s, 3H).

Step 12: (1R,2S,3R,5R)-3-(4-chloro-7H-pyrrolo[2,3-d]pyrimidin-7-yl)-5-(hydroxymethyl)-1-methylcyclopentane-1,2-diol (2.03 g, 6.8 mmol) was co-evaporated with dry toluene (10 mL×3) and then re-dissolved in acetone (20 mL). To this solution were added 4-methylbenzenesulfonic acid (0.12 g, 0.68 mmol), followed by 2,2-dimethoxypropane (3.55 g, 34.1 mmol). The resulting mixture was stirred at 25° C. for 1 hour. Then the solution was neutralized with saturated aqueous NaHCO₃ to pH ˜7 to 8. The resulting mixture was extracted with EtOAc (30 mL×3). The combined organic layers were washed with brine (50 mL), dried over anhydrous sodium sulfate, and filtered. The filtrate was concentrated under reduced pressure. The residue was purified by silica gel column chromatography and eluted with 0-70% ethyl acetate in petroleum ether to afford ((3aR,4R,6R,6aS)-6-(4-chloro-7H-pyrrolo[2,3-d]pyrimidin-7-yl)-2,2,3a-trimethyltetrahydro-3aH-cyclopenta[d][1,3]dioxol-4-yl)methanol. MS: 338 (M+1). ¹H NMR (400 MHz, DMSO-d₆) δ 8.67 (s, 1H), 7.94 (d, J=3.6 Hz, 1H), 6.74 (d, J=3.6 Hz, 1H), 5.20-5.10 (m, 1H), 4.56 (t, J=5.2 Hz, 1H), 4.41 (d, J=4.4 Hz, 1H), 3.64 (dt, J=10.5, 5.2 Hz, 1H), 3.54-3.35 (m, 1H), 2.44-2.26 (m, 2H), 2.28-2.11 (m, 1H), 1.49 (s, 6H), 1.26 (s, 3H).

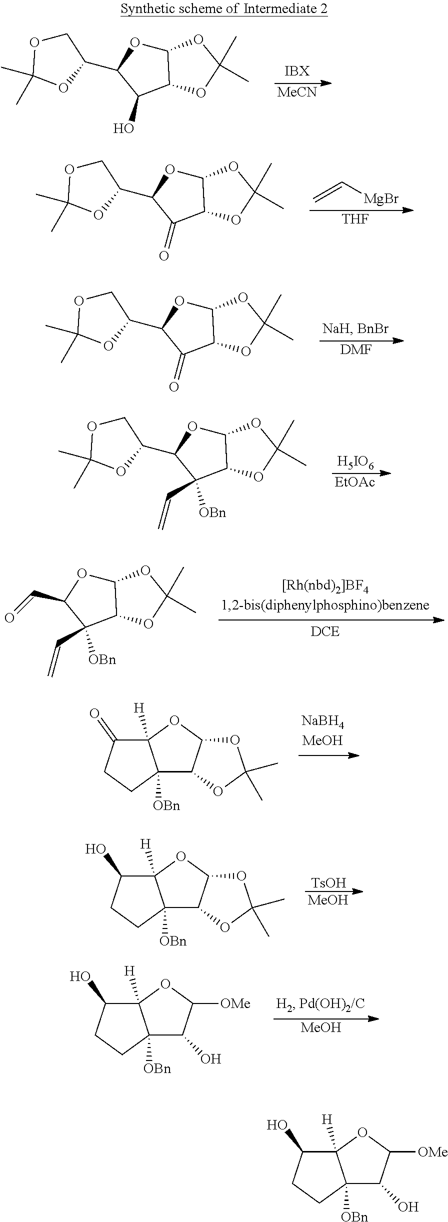

### Intermediate 2: (3R,3aS,6R,6aR)-2-methoxyhexahydro-3aH-cyclopenta[b]furan-3,3a,6-triol

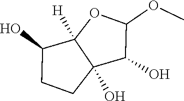

Step 1: To a solution of (3aR,5S,6S,6aR)-5-((R)-2,2-dimethyl-1,3-dioxolan-4-yl)-2,2-dimethyltetrahydrofuro[2,3-d][1,3]dioxol-6-ol (500 g, 1.92 mol) in MeCN (2.50 L) at 25° C. was added slowly IBX (807 g, 2.88 mol) at 2025° C. The reaction mixture was stirred at 8590° C. for 3 hours. The mixture was filtered and concentrated. The crude product (3aR,5R,6aS)-5-((R)-2,2-dimethyl-1,3-dioxolan-4-yl)-2,2-dimethyldihydrofuro[2,3-d][1,3]dioxol-6(5H)-one was used in the next step without further purification. ¹H NMR (400 MHz, CDCl₃) δ: 6.14 (d, J=4.4 Hz, 1H), 4.31-4.45 (m, 3H), 4.00-4.06 (m, 2H), 1.46 (s, 3H), 1.43 (s, 3H), 1.34 (s, 6H).

Step 2: To a solution of (3aR,5R,6aS)-5-((R)-2,2-dimethyl-1,3-dioxolan-4-yl)-2,2-dimethyldihydrofuro[2,3-d][1,3]dioxol-6(5H)-one (500 g, 1.94 mol) in dry THF (2.50 L) cooled to 0˜5° C. was added vinyl magnesium bromide (1 M, 3.87 L) maintaining the temperature at 0˜5° C. The reaction was warmed to 15˜20° C. and stirred for 0.5 hours. The reaction mixture was quenched by pouring into aqueous NH₄Cl (10 L) at 0-5° C. The aqueous phase was extracted with MTBE (3 L×3). The combined organic phase was washed with brine (2 L), dried with anhydrous Na₂SO₄, filtered, and concentrated under reduced pressure. The residue was purified by column chromatography (silica, petroleum ether/ethyl acetate=1/0 to 5/1) to give (3aR,5R,6aR)-5-((R)-2,2-dimethyl-1,3-dioxolan-4-yl)-2,2-dimethyl-6-vinyltetrahydrofuro[2,3-d][1,3]dioxol-6-ol.

Step 3: To a solution of NaH (105 g, 2.62 mol, 60% dispersion in mineral oil) in DMF (2.75 L) at 15˜20° C. was added (3aR,5R,6aR)-5-((R)-2,2-dimethyl-1,3-dioxolan-4-yl)-2,2-dimethyl-6-vinyltetrahydrofuro[2,3-d][1,3]dioxol-6-ol (375 g, 1.31 mol) in DMF (1 L) dropwise at 15˜20° C. The reaction mixture was stirred at 55˜60° C. for 1 h, then BnBr (336 g, 1.96 mol, 233 mL) was added. The reaction mixture was stirred at 15˜20° C. for another 5 hours. The reaction was quenched by pouring the mixture into ice water (1.5 L). The resultant mixture was extracted with ethyl acetate (2 L×3). The combined organic phase was washed with aqueous NaHCO₃ (1.5 L), dried with anhydrous Na₂SO₄, filtered, and concentrated under reduced pressure. The crude product (3aR,5R,6R,6aR)-6-(benzyloxy)-5-((R)-2,2-dimethyl-1,3-dioxolan-4-yl)-2,2-dimethyl-6-vinyltetrahydrofuro[2,3-d][1,3]dioxole was used without further purification.

Step 4: To a solution of (3aR,5R,6R,6aR)-6-(benzyloxy)-5-((R)-2,2-dimethyl-1,3-dioxolan-4-yl)-2,2-dimethyl-6-vinyltetrahydrofuro[2,3-d][1,3]dioxole (400 g, 1.06 mol) in EtOAc (2 L) at 15˜20° C. was added periodic acid (250 g, 1.09 mol) and the resultant mixture was stirred for 1 hour. The reaction was filtered, and the filtrate was concentrated under reduced pressure. The residue was purified by column chromatography (silica, petroleum ether/ethyl acetate=20/1 to 0/1) to afford (3aR,5S,6R,6aR)-6-(benzyloxy)-2,2-dimethyl-6-vinyltetrahydrofuro[2,3-d][1,3]dioxole-5-carbaldehyde. ¹H NMR (400 MHz, CDCl₃) δ: 9.58 (s, 1H), 7.26-7.43 (m, 5H), 5.97 (d, J=3.20 Hz, 1H), 5.78-5.76 (m, 1H), 5.38-5.54 (m, 2H), 4.59-4.73 (m, 4H), 1.62 (s, 3H), 1.40 (s, 3H)

Step 5: To a suspension of [Rh(nbd)₂]BF₄ (6.14 g, 16.4 mmol) in DCE (60 mL) at 15˜20° C. under N₂ was added 1,2-bis(diphenylphosphino)benzene (6.10 g, 13.7 mmol). The suspension was degassed under reduced pressure, purged with H₂ three times, and the H₂ was bubbled through the solution for 0.25 hours. The reaction mixture was flushed again with N₂ for 0.25 hours to remove H₂ (3aR,5S,6R,6aR)-6-(benzyloxy)-2,2-dimethyl-6-vinyltetrahydrofuro[2,3-d][1,3]dioxole-5-carbaldehyde (50.0 g, 164 mmol) in DCE (60 mL) was added dropwise to the above solution at 15˜20° C. under N₂. The mixture was stirred at 75-80° C. for 12 hours. The mixture was filtered, and solvent was removed to give crude (3aR,4aS,7aS,7bR)-7a-(benzyloxy)-2,2-dimethylhexahydro-5H-cyclopenta[4,5]furo[2,3-d][1,3]dioxol-5-one. ¹H NMR: (400 MHz, CDCl₃) δ: 7.5-7.25 (m, 5H), 5.94 (m, 1H), 4.69 (m, 1H), 4.63-4.57 (m, 2H), 4.18 (s, 1H), 2.40-2.56 (m, 3H), 1.68-1.74 (m, 1H), 1.61 (s, 3H), 1.40 (s, 3H)

Step 6: NaBH₄ (37.3 g, 986 mmol) was added to a mixture of (3aR,4aS,7aS,7bR)-7a-(benzyloxy)-2,2-dimethylhexahydro-5H-cyclopenta[4,5]furo[2,3-d][1,3]dioxol-5-one (150 g, 493 mmol) in MeOH (750 mL) at 0˜5° C. The mixture was stirred at 0˜5° C. for 1 hour. The residue was poured into ice-water (250 mL), and the aqueous phase was extracted with ethyl acetate (250 mL×3). The combined organic phases were washed with brine (125 mL), dried with anhydrous Na₂SO₄, filtered, and concentrated under reduced pressure. The residue was purified by column chromatography (silica, petroleum ether/ethyl acetate=20/1 to 0/1) to afford (3aR,4aR,5R,7aR,7bR)-7a-(benzyloxy)-2,2-dimethylhexahydro-5H-cyclopenta[4,5]furo[2,3-d][1,3]dioxol-5-ol. ¹H NMR: (400 MHz, CDCl₃) δ: 7.33-7.40 (m, 5H), 5.88 (d, J=3.6 Hz, 1H), 4.65 (d, J=10.8 Hz, 1H), 4.56 (d, J=3.2 Hz, 1H), 4.45-4.51 (m, 2H), 4.18-4.29 (m, 1H), 2.06-2.26 (m, 3H), 1.66-1.77 (m, 1H), 1.66-1.77 (m, 1H), 1.62 (s, 3H), 1.41 (s, 3H)

Step 7: To a solution of TsOH (10.8 g, 62.7 mmol) in MeOH (150 mL) at 15˜20° C. was added 3aR,4aR,5R,7aR,7bR)-7a-(benzyloxy)-2,2-dimethylhexahydro-5H-cyclopenta[4,5]furo[2,3-d][1,3]dioxol-5-ol (30.0 g, 97.9 mmol). The mixture was stirred at 15˜20° C. for 12 hours. The reaction was poured into ice water (16 mL) and neutralized with aqueous Na₂CO₃ (25 mL). The aqueous phase was extracted with ethyl acetate (100 mL×4). The combined organic fractions were washed with brine (100 mL), dried with anhydrous Na₂SO₄, filtered, and concentrated under reduced pressure. The residue was purified by column chromatography (silica, petroleum ether/ethyl acetate=20/1 to 0/1) to give (3R,3aS,6R,6aR)-3a-(benzyloxy)-2-methoxyhexahydro-2H-cyclopenta[b]furan-3,6-diol. ¹H NMR: (400 MHz, CDCl₃) δ: 7.26-7.40 (m, 5H), 4.93-5.03 (m, 1H), 4.52-4.76 (m, 1H), 4.33-4.45 (m, 1H), 4.00-4.19 (m, 1H), 3.78-3.97 (m, 1H), 3.46 (d, J=7.6 Hz, 3H), 2.98-3.04 (m, 1H), 2.20-2.34 (m, 1H), 1.82-2.12 (m, 4H).

Step 8: Pd(OH)₂/C (1.70 g, 2.42 mmol, 20 wt. % loading) was added to (3R,3aS,6R,6aR)-3a-(benzyloxy)-2-methoxyhexahydro-2H-cyclopenta[b]furan-3,6-diol (17.0 g, 60.7 mmol) in MeOH (150 mL) at 15˜20° C. under N₂ followed by addition of acetic acid (2.98 g, 49.5 mmol, 2.83 mL). The suspension was degassed under reduced pressure and purged with H₂ several times. The mixture was then stirred under H₂ (50 psi) at 50˜55° C. for 12 hours. The reaction mixture was filtered and concentrated under reduced pressure to afford (3R,3aS,6R,6aR)-2-methoxyhexahydro-3aH-cyclopenta[b]furan-3,3a,6-triol. ¹H NMR: (400 MHz, CDCl₃) δ: 5.00 (d, J=4.0 Hz, 1H), 4.94 (d, J=2.0 Hz, 1H), 4.22-4.18 (m, 1H), 4.16-4.13 (m, 2H), 3.76-3.83 (m, 1H), 3.47 (d, J=13.8 Hz, 3H), 2.14-1.96 (m, 2H), 1.81-1.62 (m, 2H).

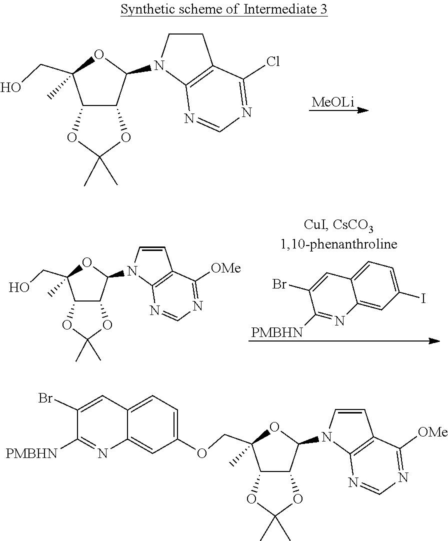

### Intermediate 3: 3-bromo-7-(((3aS,4R,6R,6aR)-6-(4-methoxy-7H-pyrrolo[2,3-d]pyrimidin-7-yl)-2,2,4-trimethyltetrahydrofuro[3,4-d][1,3]dioxol-4-Yl)methoxy)-N-(4-methoxybenzyl)quinolin-2-amine

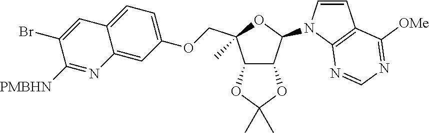

Step 1: To a solution of ((3aS,4R,6R,6aR)-6-(4-chloro-7H-pyrrolo[2,3-d]pyrimidin-7-yl)-2,2,4-trimethyltetrahydrofuro[3,4-d][1,3]dioxol-4-yl)methanol (95.0 mg, 0.280 mmol) in methanol (1.0 mL) was added LiOMe (106 mg, 2.80 mmol). The reaction mixture was stirred at room temperature for 20 minutes and then diluted with water (10 mL). The resulting mixture was extracted with DCM (10 mL) and organic layers were dried over Na₂SO₄. The organic solvent was removed under reduced pressure to yield ((3aS,4R,6R,6aR)-6-(4-methoxy-7H-pyrrolo[2,3-d]pyrimidin-7-yl)-2,2,4-trimethyltetrahydrofuro[3,4-d][1,3]dioxol-4-yl)methanol. The product was used for the next step without further purification. MS: 336 (M+1). 1H NMR (500 MHz, CDCl₃) δ 8.43 (s, 1H), 7.07 (d, J=3.6 Hz, 1H), 6.89 (dd, J=12.1, 1.7 Hz, 1H), 6.51 (d, J=3.6 Hz, 1H), 5.74 (d, J=5.7 Hz, 1H), 5.35 (t, J=5.8 Hz, 1H), 5.01 (d, J=5.9 Hz, 1H), 4.12 (s, 3H), 3.80 (dd, J=12.2, 1.7 Hz, 1H), 3.63 (t, J=12.2 Hz, 1H), 1.65 (s, 3H), 1.37 (s, 3H), 1.35 (s, 3H).

Step 2: To a solution of ((3aS,4R,6R,6aR)-6-(4-methoxy-7H-pyrrolo[2,3-d]pyrimidin-7-yl)-2,2,4-trimethyltetrahydrofuro[3,4-d][1,3]dioxol-4-yl)methanol (94.0 mg, 0.280 mmol), 3-bromo-7-iodo-N-(4-methoxybenzyl)quinolin-2-amine (75.0 mg, 0.160 mmol), copper iodide (3.05 mg, 0.0160 mmol) and 1,10-phenanthroline (5.77 mg, 0.0320 mmol) in dioxanes (0.250 mL) was added cesium carbonate (78.0 mg, 0.240 mmol). The resulting mixture was stirred at 110° C. for 23 hours. The reaction mixture was directly purified by flash column chromatography (EtOAc in hexanes, 0-25%) to yield 3-bromo-7-(((3aS,4R,6R,6aR)-6-(4-methoxy-7H-pyrrolo[2,3-d]pyrimidin-7-yl)-2,2,4-trimethyltetrahydrofuro[3,4-d][1,3]dioxol-4-yl)methoxy)-N-(4-methoxybenzyl)quinolin-2-amine as a solid. MS: 676/678 (M+1/M+3). ¹H-NMR (500 MHz, Chloroform-d) δ 8.51 (s, 1H), 8.01 (s, 1H), 7.42 (d, J=8.8 Hz, 1H), 7.36 (d, J=8.6 Hz, 2H), 7.32 (d, J=3.7 Hz, 1H), 7.06 (d, J=2.4 Hz, 1H), 6.90 (d, J=8.7 Hz, 2H), 6.81 (dd, J=8.8, 2.5 Hz, 1H), 6.49 (d, J=3.7 Hz, 1H), 6.44 (d, J=3.7 Hz, 1H), 5.56 (t, J=5.1 Hz, 1H), 5.44 (dd, J=6.1, 3.8 Hz, 1H), 5.00 (d, J=6.2 Hz, 1H), 4.72 (d, J=5.1 Hz, 2H), 4.21 (d, J=9.7 Hz, 1H), 4.09 (s, 3H), 4.05 (d, J=9.7 Hz, 1H), 3.81 (s, 3H), 1.71 (s, 3H), 1.55 (s, 3H), 1.42 (s, 3H).

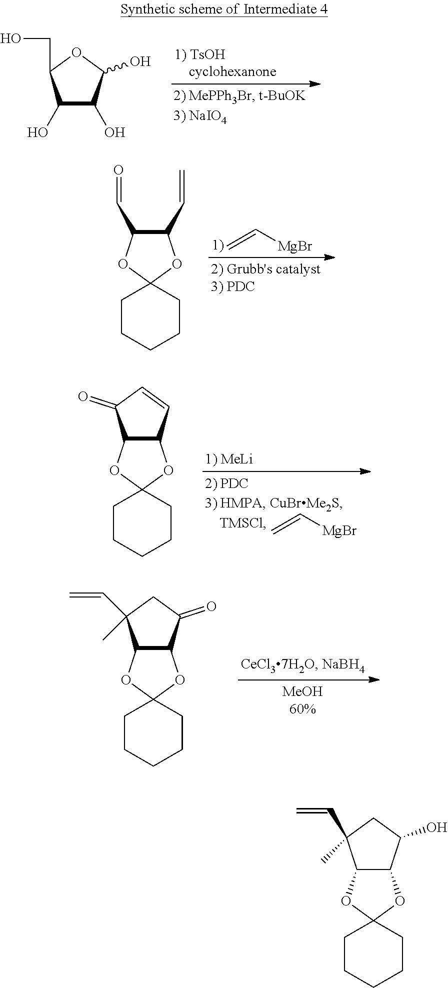

### Intermediate 4: (3a′R,4′R,6'S,6a'S)-4′-methyl-4′-vinyltetrahydro-4′H-spiro[cyclohexane-1,2′-cyclopenta[d][1,3]dioxol]-6′-ol

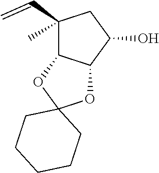 [SMILES: C=C[C@@]1(C)C[C@H](O)[C@@H]2OC3(CCCCC3)O[C@@H]21]

Step 1: Into a 10-L 4-necked round-bottom flask, purged and maintained with an inert atmosphere of nitrogen were placed D-ribofuranose (970 g, 6.46 mol), cyclohexanone (6.4 L), and 4-methylbenzene-1-sulfonic acid (22.8 g, 132 mmol). The resulting solution was stirred overnight at 25° C. The resulting solution was extracted with 5 L of ethyl acetate and the organic layers combined. The organic layers were washed with 5 L of saturated aqueous NaHCO₃ solution and 5 L of H₂O. The organic layers were dried over sodium sulfate. The resulting mixture was concentrated under reduced pressure. The residue was purified by silica gel column chromatography (EtOAc/petroleum ether (1:1)) to afford 2,3-O-1,1-cyclohexanediyl-D-ribofuranose.

Step 2: Into a 20-L 4-necked round-bottom flask, purged and maintained with an inert atmosphere of nitrogen were placed MePPh₃Br (1.83 kg, 5.13 mol) and tetrahydrofuran (12.7 L). This was followed by the addition of t-BuOK (657 g, 5.86 mol) at 0° C. in 15 min. To this mixture was added 2,3-O-1,1-cyclohexanediyl-D-ribofuranose (422 g, 1.83 mol) at 0° C. The resulting solution was stirred for 1 hours at 25° C. The reaction was quenched by the addition of 20 L of water. The resulting solution was extracted with 20 L of ethyl acetate, and the organic layers were combined and dried over sodium sulfate. The resulting mixture was filtered and concentrated under reduced pressure. The residue was purified by column chromatography on silica (EtOAc/petroleum ether (1:3)) to afford (R)-1-((2R,3S)-3-vinyl-1,4-dioxaspiro[4.5]decan-2-yl)ethane-1,2-diol.

Step 3: Into a 20-L 4-necked round-bottom flask, purged and maintained with an inert atmosphere of nitrogen were placed (R)-1-((2R,3S)-3-vinyl-1,4-dioxaspiro[4.5]decan-2-yl)ethane-1,2-diol (630 g, 2.76 mol) and dichloromethane (8.19 L). This was followed by the dropwise addition of a solution of sodium periodate (588 g, 2.75 mol) in water (4.41 L). The resulting mixture was stirred for 30 minutes at 25° C. The solids were filtered off and the filtrate was concentrated under reduced pressure. The residue was purified via a silica gel column with ethyl acetate/petroleum ether (1:10). The product containing fractions were combined and concentrated under reduced pressure to afford (2S,3S)-3-vinyl-1,4-dioxaspiro[4.5]decane-2-carbaldehyde.

Step 4: Into a 20-L 4-necked round-bottom flask, purged and maintained with an inert atmosphere of nitrogen were placed (2S,3S)-3-vinyl-1,4-dioxaspiro[4.5]decane-2-carbaldehyde (637 g, 3.25 mol) and tetrahydrofuran (7.96 L). This was followed by the dropwise addition of bromo(ethenyl)magnesium (4.88 L, 1 M in THF) with stirring at 0° C. The resulting mixture was stirred for 10 minutes at 0° C., and then warmed to room temperature and allowed to stir for an additional 1 hour at 25° C. The reaction was quenched by the addition of 7 L of saturated aqueous NH₄Cl solution. The resulting solution was extracted with 7 L of ethyl acetate, and the combined organic layers were dried over sodium sulfate, filtered, and concentrated under reduced pressure. The residue was purified via a silica gel column with ethyl acetate/petroleum ether (1:50) to afford (R)-1-((2S,3R)-3-vinyl-1,4-dioxaspiro[4.5]decan-2-yl)prop-2-en-1-ol.

Step 5: Into a 20-L 4-necked round-bottom flask, purged and maintained with an inert atmosphere of nitrogen were placed (R)-1-((2S,3R)-3-vinyl-1,4-dioxaspiro[4.5]decan-2-yl)prop-2-en-1-ol (400 g, 1.78 mol), dichloromethane (12.8 L), and Grubbs catalyst (24.3 g). The mixture was stirred for 24 hours at 25° C. To the mixture were added PDC (1.34 kg, 3.57 mol) and 4 Å molecular sieves (400 g). The resulting mixture was stirred for 4 hours at 25° C. The solids were filtered off, and the filtrate was concentrated under reduced pressure. The residue was purified by column chromatography on silica (EtOAc/petroleum ether (1:40)) to afford (3a'S,6a'S)-3a′,6a′-dihydro-4′H-spiro[cyclohexane-1,2′-cyclopenta[d][1,3]dioxol]-4′-one.

Step 6: Into a 10-L 4-necked round-bottom flask, purged and maintained with an inert atmosphere of nitrogen, were placed (3a'S,6a'S)-3a′,6a′-dihydro-4′H-spiro[cyclohexane-1,2′-cyclopenta[d][1,3]dioxol]-4′-one (246 g, 1.27 mol) and tetrahydrofuran (3.44 L). To this stirring mixture at −78° C. was added methyllithium (1.74 L, 2.79 mol, 1.6 M in diethyl ether) dropwise. The mixture was stirred for 30 minutes at −78° C., then allowed to warm to room temperature and continued to stir for an additional 1 hours at 25° C. The reaction was quenched by the addition of 3 L of saturated aqueous NH₄Cl solution. The resulting solution was extracted with 3 L of ethyl acetate and the organic layers were combined and dried over sodium sulfate, filtered, and concentrated under reduced pressure. The residue was purified by column chromatography on silica (EtOAc/petroleum ether (1:20)) to afford (3a'S,4′R,6a'S)-4′-methyl-4′,6a′-dihydro-3a′H-spiro[cyclohexane-1,2′-cyclopenta[d][1,3]dioxol]-4′-ol.

Step 7: Into a 10-L 3-necked round-bottom flask, purged and maintained with an inert atmosphere of nitrogen were placed (3a'S,4′R,6a'S)-4′-methyl-4′,6a′-dihydro-3a′H-spiro[cyclohexane-1,2′-cyclopenta[d][1,3]dioxol]-4′-ol (192 g, 913 mmol), dichloromethane (3.84 L), 4 Å molecular sieves (192 g), PDC (688 g, 1.83 mol), and acetic anhydride (747 g, 7.3 mol). The mixture was stirred overnight at 25° C. The reaction was quenched by the addition of 1 L of saturated aqueous Na₂CO₃ solution. The resulting solution was extracted with 1 L of dichloromethane and the combined organic layers were dried over sodium sulfate, filtered, and concentrated under reduced pressure. The residue was purified by column chromatography on silica (EtOAc/petroleum ether (1:50)) to afford (3a′R,6a′R)-6′-methyl-3a′,6a′-dihydro-4′H-spiro[cyclohexane-1,2′-cyclopenta[d][1,3]dioxol]-4′-one.

Step 8: Into a 2-L 3-necked round-bottom flask, purged and maintained with an inert atmosphere of nitrogen, was placed CuBrMe₂S (8.43 g, 41.1 mmol) and tetrahydrofuran (627 mL). This was followed by the dropwise addition of bromo(ethenyl)magnesium (548 mL, 2 M in THF, 548 mmol) with stirring at −78° C. To this mixture was added HMPA (294 g, 1.64 mol) at −78° C., then (3a′R,6a′R)-6′-methyl-3a′,6a′-dihydro-4′H-spiro[cyclohexane-1,2′-cyclopenta[d][1,3]dioxol]-4′-one (57.0 g, 274 mmol) and chlorotrimethylsilane (148 g, 1.36 mol). The resulting mixture was stirred for 3 hours at −78° C. The reaction was quenched by the addition of 500 mL of saturated aqueous NH₄Cl solution. The resultant mixture was extracted with 1 L of ethyl acetate the combined organic layers were dried over sodium sulfate, filtered, and concentrated under reduced pressure. The residue was purified by column chromatography on silica (EtOAc/petroleum ether (1:100)) to afford (3a′R,6′R,6a′R)-6′-methyl-6′-vinyltetrahydro-4′H-spiro[cyclohexane-1,2′-cyclopenta[d][1,3]dioxol]-4′-one.

Step 9: Into a 2-L 3-necked round-bottom flask, purged and maintained with an inert atmosphere of nitrogen were placed (3a′R,6′R,6a′R)-6′-methyl-6′-vinyltetrahydro-4′H-spiro[cyclohexane-1,2′-cyclopenta[d][1,3]dioxol]-4′-one (32.2 g, 136 mmol) and methanol (966 mL). To this mixture was added CeCl₃.7H₂O (50.8 g) at −30° C., then NaBH₄ (10.3 g, 273 mmol). The resulting mixture was stirred for 15 minutes at −30° C., then allowed to warm to room temperature, and the stirring was continued for an additional 30 minutes at 25° C. The reaction was quenched by the addition of 1 L ethyl acetate/petroleum ether (1:1). The solids were filtered off. The filtrate was concentrated under reduced pressure. The residue was purified by column chromatography on silica (EtOAc/petroleum ether (1:70)) to afford (3a′R,4′R,6'S,6a'S)-4′-methyl-4′-vinyltetrahydro-4′H-spiro[cyclohexane-1,2′-cyclopenta[d][1,3]dioxol]-6′-ol. ¹H NMR (300 MHz, CDCl₃) δ 5.72-5.66 (m, 1H), 5.03-4.99 (m, 2H), 4.45 (t, J=6.0 Hz, 1H), 4.32 (d, J=5.5 Hz, 1H), 4.03-3.99 (m, 1H), 2.51 (d, J=10.0 Hz, 1H), 1.98-1.94 (m, 1H), 1.72-1.52 (m, 9H), 1.43-1.38 (m, 2H), 1.12 (s, 3H).

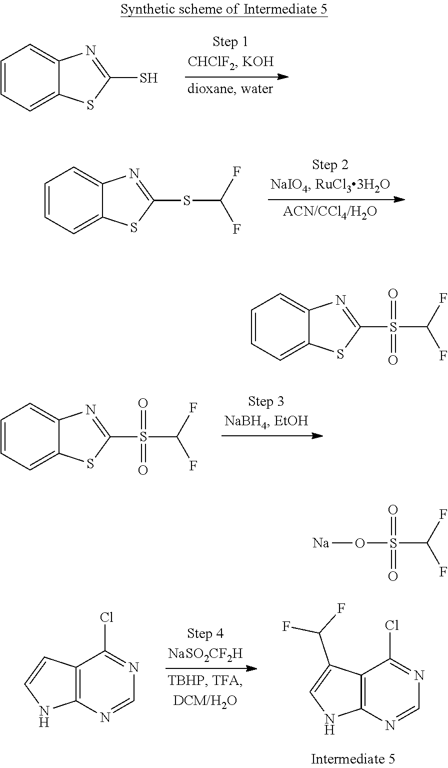

### Intermediate 5: 4-chloro-5-(difluoromethyl)-7H-pyrrolo[2,3-d]pyrimidine

Step 1: To a solution of benzo[d]thiazole-2-thiol (50 g, 300 mmol) in 1,4-dioxane (125 mL) and water (125 mL) was added potassium hydroxide (30 g, 540 mmol) at 0° C. Excess chlorodifluoromethane was bubbled through the resulting mixture over 5 h. The reactor was sealed, and the mixture was stirred at room temperature for 8 h before being concentrated under reduced pressure. The residue was purified by column chromatography on silica (neutralized with triethylamine) (0-30% ethyl acetate/hexanes) to give 2-((difluoromethyl)thio)benzo[d]thiazole. MS: 218 (M+1). ¹H-NMR (300 MHz, Chloroform-d) δ 8.05-8.02 (m, 1H), 7.89-7.86 (m, 1H), 7.67-7.41 (m, 3H). ¹⁹F-NMR (282 MHz, Chloroform-d) δ −93.20 (s, CF₂H).

Step 2: To a solution of 2-((difluoromethyl)thio)benzo[d]thiazole (11.1 g, 51 mmol) in a mixture of ACN/CCl₄/water (v:v:v=1:1:2, 222 mL) were added sodium periodate (34.2 g, 160 mmol) and ruthenium(III) chloride trihydrate (33 mg, 0.13 mmol) portion wise. The resulting solution was stirred at room temperature for 3 h. The mixture was diluted with water (800 mL) and extracted with DCM (1500 mL). The organic phase was washed with brine (800 mL), dried over anhydrous sodium sulfate, and filtered. The filtrate was concentrated under reduced pressure, and the residue was purified by column chromatography on silica (0-20% ethyl acetate/DCM) to give 2-((difluoromethyl)sulfonyl)benzo[d]thiazole. MS: 250 (M+1). ¹H-NMR (400 MHz, Chloroform-d) δ 8.38-8.33 (m, 1H), 8.15-8.06 (m, 1H), 7.76-7.69 (m, 2H), 6.62 (t, J=52 Hz, 1H). ¹⁹F-NMR (376 MHz, Chloroform-d) δ −121.39 (s, CF₂H).

Step 3: To a solution of 2-((difluoromethyl)sulfonyl)benzo[d]thiazole (116.5 g, 467 mmol) in ethanol (700 mL) was added sodium borohydride (26.5 g, 700 mmol) portion wise at room temperature under argon atmosphere. The mixture was stirred at room temperature for 2 h. The reaction mixture was concentrated under reduced pressure. The crude material was triturated with hexane (600 mL×3) at room temperature to afford sodium difluoromethanesulfinate. ¹H-NMR (400 MHz, Methanol-d₄) δ 5.14 (t, J=56 Hz, 1H). ¹⁹F-NMR (376 MHz, Methanol-d₄) δ −128.92 (s, CF₂H).

Step 4: To a solution of 4-chloro-7H-pyrrolo[2,3-d]pyrimidine (10 g, 65.1 mmol) in DCM (150 mL) and water (60 mL) were added sodium difluoromethanesulfinate (27 g, 195 mmol) and TFA (10.0 mL, 130 mmol) portion wise at 0° C. To this mixture was dropwise added tert-butyl hydroperoxide (5.5M in decane, 59 mL, 330 mmol), and the resulting mixture was stirred at room temperature for 5 days before being quenched with sodium bicarbonate (2 M aq, 110 mL). The mixture was extracted with DCM (200 mL×3). The combined organic layers were washed with brine (200 mL×3), dried over anhydrous sodium sulfate, filtered, and the filtrate was concentrated under reduced pressure. The product was purified by column chromatography (0-20% ethyl acetate/hexanes) to give 4-chloro-5-(difluoromethyl)-7H-pyrrolo[2,3-d]pyrimidine. MS: 204 (M+1). ¹H-NMR (400 MHz, DMSO-d₆) δ 13.40 (br s, 1H), 8.73 (s, 1H), 7.31 (t, J=54 Hz, 1H), 7.00-7.00 (m, 1H). ¹⁹F-NMR (376 MHz, DMSO-d₆) δ −112.14 (s, CF₂H).

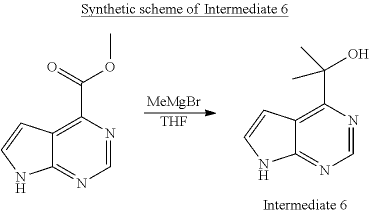

### Intermediate 6: 2-(7H-pyrrolo[2,3-d]pyrimidin-4-yl)propan-2-ol

Methyl 7H-pyrrolo[2,3-d]pyrimidine-4-carboxylate (0.52 g, 2.9 mmol) dissolved in tetrahydrofuran (12 mL) was purged with nitrogen and cooled to −78° C. To the solution was added methylmagnesium bromide (1.4 M, 4.6 mL, 6.5 mmol), and the reaction was warmed to room temperature and stirred for 1 h. After 2 hours additional methylmagnesium bromide (1.4 M, 4.6 mL, 6.5 mmol) was added at −78° C., and the reaction stirred for 18 h and warmed to room temperature. The reaction was quenched with saturated aq. ammonium chloride and stirred for 1 h. at room temperature. The organics were separated, washed with brine, dried over sodium sulfate, and the solvents were removed under reduced pressure to afford 2-(7H-pyrrolo[2,3-d]pyrimidin-4-yl)propan-2-ol, which was used without further purification. MS: 178 (M+1).

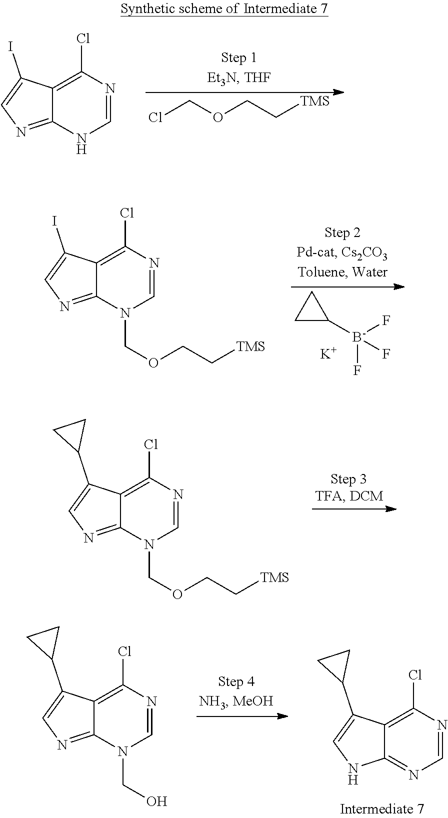

### Intermediate 7: 4-Chloro-5-cyclopropyl-1H-pyrrolo[2,3-d]pyrimidine

Step 1: To a stirred mixture of 4-chloro-5-iodo-1H-pyrrolo[2,3-d]pyrimidine (10.0 g, 35.8 mmol) in THF (119 mL) was added triethylamine (12.5 mL, 89.0 mmol) and (2-(chloromethoxy)ethyl)trimethylsilane (7.60 mL, 42.9 mmol) at 0° C. The mixture was warmed to room temperature and stirred overnight. The mixture was treated with water and extracted with EtOAc. The combined organics were washed with brine, dried over sodium sulfate, concentrated under reduced pressure, and purified by column chromatography on silica (0-10% EtOAc/DCM) to afford 4-chloro-5-iodo-7-{[2-(trimethylsilyl)ethoxy]methyl}-7H-pyrrolo[2,3-d]pyrimidine. MS: 410 (M+1). ¹H NMR (500 MHz, DMSO-d₆) δ 8.69 (s, 1H), 8.13 (s, 1H), 5.60 (s, 2H), 3.57-3.45 (m, 2H), 0.87-0.75 (m, 2H), −0.10 (s, 9H).

Step 2: A mixture of 4-chloro-5-iodo-7-{[2-(trimethylsilyl)ethoxy]methyl}-7H-pyrrolo[2,3-d]pyrimidine (12.2 g, 29.8 mmol), potassium cyclopropyltrifluoroborate (5.29 g, 35.7 mmol), cesium carbonate (29.1 g, 89.0 mmol), and [(di(1-adamantyl)-butylphosphine)-2-(2′-amino-1,1′-biphenyl)]palladium(II) methanesulfonate (2.17 g, 2.98 mmol) in toluene (135 mL)/water (13.5 mL) was purged with nitrogen and then stirred at 100° C. for 10 h. The mixture was cooled to room temperature, diluted with EtOAc, and washed with water and brine. The organic layer was dried over sodium sulfate, concentrated, and purified by column chromatography on silica (0-20% EtOAc/DCM) to afford 4-chloro-5-cyclopropyl-7-{[2-(trimethylsilyl)ethoxy]methyl}-7H-pyrrolo[2,3-d]pyrimidine. MS: 324 (M+1).

Step 3: To a stirred solution of 4-chloro-5-cyclopropyl-7-{[2-(trimethylsilyl)ethoxy]methyl}-7H-pyrrolo[2,3-d]pyrimidine (7.35 g, 22.7 mmol) in DCM (91 mL) was added TFA (14.0 mL, 182 mmol). The mixture was stirred at 32° C. overnight. The mixture was cooled to room temperature, concentrated, diluted with EtOAc, and washed with saturated sodium bicarbonate solution. The organic layer was dried over sodium sulfate, concentrated, and purified by silica gel chromatography (0-100% EtOAc in DCM) to afford (4-chloro-5-cyclopropyl-7H-pyrrolo[2,3-d]pyrimidin-7-yl)methanol. MS: 224 (M+1). ¹H NMR (500 MHz, DMSO-d₆) δ 8.59 (s, 1H), 7.49-7.38 (m, 1H), 6.73-6.59 (m, 1H), 5.53 (d, J=4.6 Hz, 2H), 2.20-2.09 (m, 1H), 1.00-0.85 (m, 2H), 0.71-0.59 (m, 2H).

Step 4: To (4-chloro-5-cyclopropyl-7H-pyrrolo[2,3-d]pyrimidin-7-yl)methanol (3.40 g, 15.2 mmol) was added ammonia (7 N in MeOH, 58.6 mL, 410 mmol). The solution was left to stir for 10 min, concentrated, and purified by column chromatography on silica (0-100% EtOAc/DCM) to afford 4-chloro-5-cyclopropyl-1H-pyrrolo[2,3-d]pyrimidine. MS: 194 (M+1). ¹H NMR (600 MHz, DMSO-d₆) δ 12.23 (s, 1H), 8.51 (s, 1H), 7.34 (d, J=0.8 Hz, 1H), 2.17-2.09 (m, 1H), 0.91-0.86 (m, 2H), 0.68-0.62 (m, 2H).

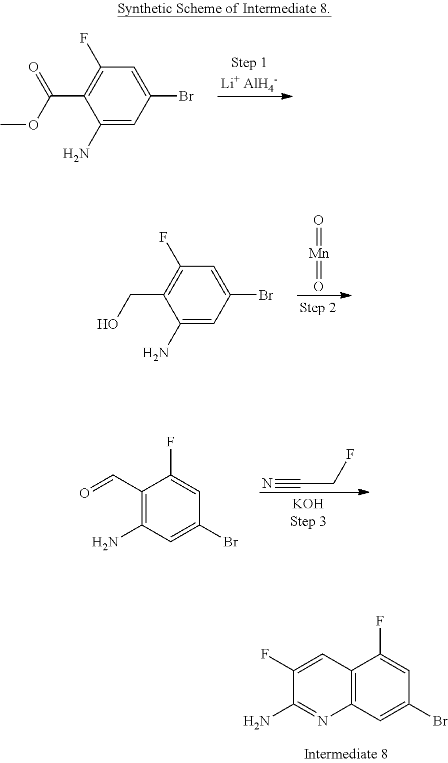

### Intermediate 8: 7-bromo-3,5-difluoroquinolin-2-amine

Step 1: Methyl 2-amino-4-bromo-6-fluorobenzoate (5.0 g, 20 mmol) was dissolved in THF (40 mL) under an atmosphere of nitrogen and cooled to 0° C. Lithium Aluminum Hydride (1M in THF, 40.3 mL, 40.3 mmol) was added dropwise to the stirring solution. The reaction was stirred for 3 h and cooled to 0° C. The reaction was quenched with sequential dropwise additions of water (2 mL), sodium hydroxide (1N in water, 3 mL), and water (6 mL). Magnesium sulfate was then added and stirred for 30 minutes. The solution was filtered through a pad of Celite® and the solvent removed under reduced pressure. The residue was purified by column chromatography on silica (0-30% DCM/3:1 EtOAc/EtOH) to afford (2-amino-4-bromo-6-fluorophenyl)methanol. MS: 202/204 (M−18/M−16).

Step 2: Manganese(IV) Oxide (4.27 g, 49.1 mmol) was added to a stirring solution of (2-amino-4-bromo-6-fluorophenyl)methanol (2.7 g, 12.27 mmol) in DCM (61 mL). The reaction was stirred for 18 h at 40° C. The reaction was filtered through a pad of Celite® and rinsed with EtOAc, and the solvent removed to afford 2-amino-4-bromo-6-fluorobenzaldehyde, which was used without further purification. MS: 218/220 (M+1/M+3).

Step 3: 2-Amino-4-bromo-6-fluorobenzaldehyde (1.20 g, 5.50 mmol) was dissolved in DMSO (11 mL). To the stirring solution was added 2-fluoroacetonitrile (1.2 mL, 22 mmol) and potassium hydroxide (0.055 mL, 0.83 mmol). The reaction mixture was then stirred at 80° C. for 18 h. The reaction was diluted with EtOAc, added to water, and let stir for several minutes. The aqueous layer was separated and washed with EtOAc. The combined organic layers were dried over sodium sulfate, filtered, and the solvent removed under reduced pressure. The material was purified by column chromatography on silica (0-100% EtOAc/Hexanes) to afford 7-bromo-3,5-difluoroquinolin-2-amine. MS: 259/261 (M+1/M+3). ¹H NMR (DMSO-d6) δ: 7.89 (d, J=11 Hz, 1H), 7.52 (s, 1H), 7.32 (dd, J=10, 1 Hz, 1H), 7.28 (s, 2H)

Intermediates 9-10: Intermediates 9-10 (as shown in Table 1) were synthesized using the protocol described with intermediate 8 making the appropriate substitution for the aryl-ester in step 1 or the benzylic alcohol in step 2 or the aryl-aldehyde in step 3.

TABLE 1

| Intermediate | Structure | Name | MS |
|---|---|---|---|
| 9 | 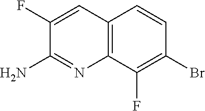 [SMILES: Nc1nc2c(F)c(Br)ccc2cc1F] | 7-bromo-3,8-difluoroquinolin- 2-amine | 259/261 (M + 1/M + 3) |
| 10 | 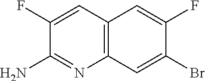 [SMILES: Nc1nc2cc(Br)c(F)cc2cc1F] | 7-bromo-3,6-difluoroquinolin- 2-amine | 259/261 (M + 1/M + 3) |

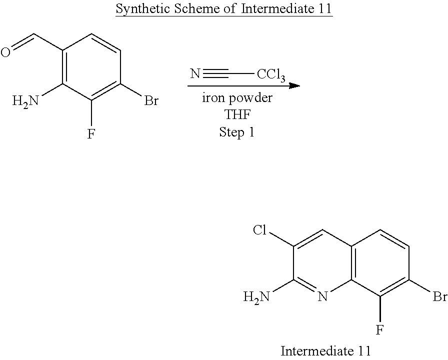

### Intermediate 11: 7-bromo-3-chloro-8-fluoroquinolin-2-amine

A flask containing 2-amino-4-bromo-3-fluorobenzaldehyde (1.8 g, 8.3 mmol) and iron powder (4.6 g, 80 mmol) was purged with nitrogen, charged with THF (16.5 mL), trichloroacetonitrile (1.2 mL, 12 mmol) and heated to 65° C. overnight. The reaction was cooled to room temperature, filtered, and washed with EtOAc. The organics were concentrated under reduced pressure, and the residue was purified by column chromatography on silica (0-100% EtOAc/DCM). The product containing fractions were concentrated under reduced pressure. The residue was dissolved in THF (50 mL), charged with N-propyldiethanolamine-functionalized silica gel (0.84 mmol/g) and allowed to stir overnight. The silica was filtered off through a pad of Celite® and washed with THF (50 mL). The organics were concentrated under reduced pressure to yield 7-bromo-3-chloro-8-fluoroquinolin-2-amine. MS: 275/277 (M+1/M+3). ¹H NMR (500 MHz, DMSO-d6) δ 8.32 (s, 1H), 7.48-7.45 (m, 1H), 7.41 (m, 1H), 7.26 (br s, 2H).

Intermediate 12: Intermediate 12 in Table 2 was synthesized using the protocol described in intermediate 11 making the appropriate substitution for the aryl-aldehyde. The substituted starting material was commercially acquired, synthesized as reported above, or synthesized through known routes reported in the literature.

TABLE 2

| Intermediate | Structure | Name | MS |
|---|---|---|---|
| 12 | 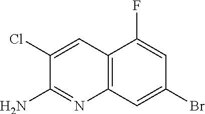 [SMILES: Nc1nc2cc(Br)cc(F)c2cc1Cl] | 7-bromo-3- chloro-5- fluoroquinolin- 2-amine | 275/ 277 (M+1/ M+3) |

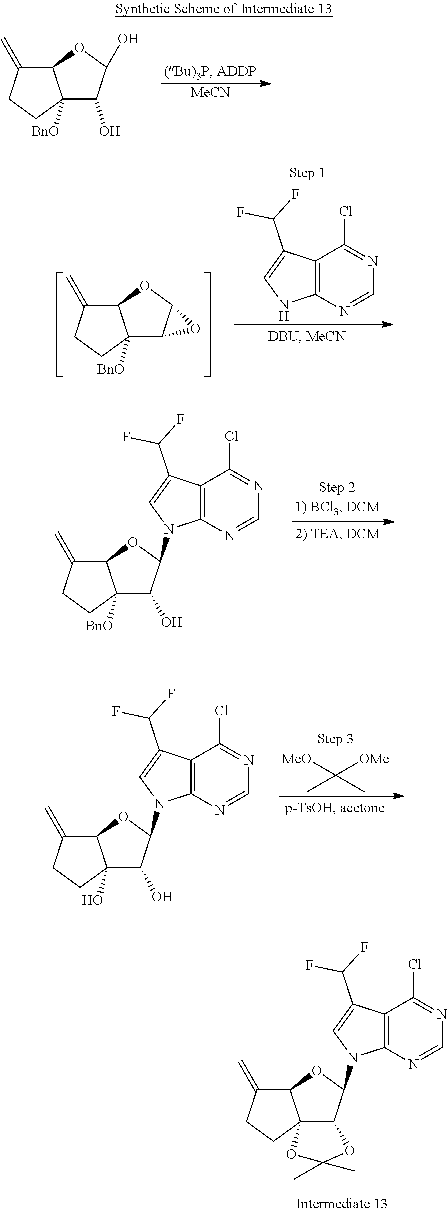

### Intermediate 13: 4-chloro-5-(difluoromethyl)-7-((3aR,4R,5aR,8aR)-2,2-dimethyl-6-methylenehexahydrocyclopenta[2,3]furo[3,4-d][1,3]dioxol-4-yl)-7H-pyrrolo[2,3-d]pyrimidine

Step 1: To a stirred solution of (3R,3aS,6aR)-3a-(benzyloxy)-6-methylenehexahydro-2H-cyclopenta[b]furan-2,3-diol (1.0 g, 3.8 mmol) in dry acetonitrile (60 mL) was dropwise added (E)-diazene-1,2-diylbis(piperidin-1-ylmethanone) (1.54 g, 6.1 mmol) at 0° C. under the atmosphere of argon, followed by tributylphosphine (1.4 mL, 5.7 mmol). The resulting mixture was stirred at 35° C. for 1 h. In a separate container, DBU (0.86 mL, 5.7 mmol) was added to a stirring solution of 4-chloro-5-(difluoromethyl)-7H-pyrrolo[2,3-d]pyrimidine (1.1 g, 5.3 mmol) in dry acetonitrile (25 mL) under the atmosphere of argon at room temperature. The resultant mixture was stirred at room temperature for 30 min. The DBU solution was transferred to the above epoxide containing solution by means of a syringe. The final mixture was stirred at 35° C. for 16 h. The reaction mixture was quenched by adding saturated aqueous ammonium chloride (150 mL) and extracted with ethyl acetate (100 mL×3). The combined organics was washed with brine (100 mL), dried over anhydrous sodium sulfate, and filtered. The filtrate was concentrated under reduced pressure. The product was purified by column chromatography on silica (0-30% ethyl acetate/petroleum ether) to give (2R,3R,3aS,6aR)-3a-(benzyloxy)-2-(4-chloro-5-(difluoromethyl)-7H-pyrrolo[2,3-d]pyrimidin-7-yl)-6-methylenehexahydro-2H-cyclopenta[b]furan-3-ol. MS: 448 (M+1). ¹H NMR (300 MHz, DMSO-d₆) δ 8.84 (s, 1H), 7.61-7.21 (m, 7H), 6.16 (d, J=8.1 Hz, 1H), 5.77 (d, J=6.3 Hz, 1H), 5.21-5.14 (m, 3H), 4.94 (d, J=12.0 Hz, 1H), 4.72-4.66 (m, 2H), 2.85-2.75 (m, 1H), 2.59-2.54 (m, 1H), 2.28-2.25 (m, 1H), 2.07-2.00 (m, 1H). ¹⁹F NMR (282 MHz, DMSO-d₆) δ −109.36 (d, 1F), −114.08 (d, 1F).

Step 2: To a solution of (2R,3R,3aS,6aR)-3a-(benzyloxy)-2-(4-chloro-5-(difluoromethyl)-7H-pyrrolo[2,3-d]pyrimidin-7-yl)-6-methylenehexahydro-2H-cyclopenta[b]furan-3-ol (1.79 g, 4.00 mmol) in DCM (20 mL) was added boron trichloride (1M in DCM, 8.0 mL, 8.0 mmol) at −78° C. under argon atmosphere. The mixture was then stirred at −78° C. for 2 h. Triethylamine (2.2 mL, 16 mmol) was carefully added at −78° C. to quench the reaction and the mixture was stirred at −78° C. for 0.5 h. The mixture was poured into saturated aqueous sodium bicarbonate (100 mL) at 0° C. The mixture was extracted with 200 mL of ethyl acetate. The organic phase was washed with water (30 mL) and brine (60 mL), dried over anhydrous sodium sulfate, and filtered. The filtrate was concentrated under reduced pressure, and the residue was purified by column chromatography on silica (0-50% ethyl acetate/petroleum ether) to give (2R,3R,3aS,6aR)-2-(4-chloro-5-(difluoromethyl)-7H-pyrrolo[2,3-d]pyrimidin-7-yl)-6-methylenehexahydro-2H-cyclopenta[b]furan-3,3a-diol. MS: 358 (M+1). ¹H NMR (400 MHz, DMSO-d₆) δ 8.81 (s, 1H), 7.59-7.09 (m, 2H), 6.07-6.04 (m, 1H), 5.50-5.47 (m, 1H), 5.31 (d, J=4.4 Hz, 1H), 5.14-5.09 (m, 2H), 4.94-4.89 (m, 1H), 4.40 (d, J=4.0 Hz, 1H), 2.80-2.67 (m, 1H), 2.51-2.41 (m, 1H), 2.13-2.06 (m, 1H), 1.71-1.65 (m, 1H). ¹⁹F NMR (376 MHz, DMSO-d₆) δ −108.88 (d, 1F), −114.52 (d, 1F).

Step 3: To a mixture of (2R,3R,3aS,6aR)-2-(4-chloro-5-(difluoromethyl)-7H-pyrrolo[2,3-d]pyrimidin-7-yl)-6-methylenehexahydro-2H-cyclopenta[b]furan-3,3a-diol (670 mg, 1.87 mmol) in acetone (12 mL) was added 2,2-dimethoxypropane (1.2 mL, 9.4 mmol) and 4-methylbenzenesulfonic acid (32 mg, 0.19 mmol) portion wise at ambient temperature. The reaction mixture was stirred at ambient temperature for 16 h. The mixture was concentrated under reduced pressure, and the residue was purified by column chromatography on silica (0-30% ethyl acetate/petroleum ether) to give 4-chloro-5-(difluoromethyl)-7-((3aR,4R,5aR,8aR)-2,2-dimethyl-6-methylenehexahydrocyclopenta[2,3]furo[3,4-d][1,3]dioxol-4-yl)-7H-pyrrolo[2,3-d]pyrimidine. MS: 398 (M+1). ¹H NMR (300 MHz, Methanol-d₄) δ 8.74 (s, 1H), 7.40-7.05 (m, 2H), 6.27 (d, J=4.5 Hz, 1H), 5.76 (d, J=4.2 Hz, 1H), 5.09-5.06 (m, 2H), 4.63 (s, 1H), 2.94-2.72 (m, 2H), 2.57-2.50 (m, 1H), 2.19-2.08 (m, 1H), 1.62 (s, 3H), 1.44 (s, 3H). ¹⁹F NMR (282 MHz, Methanol-d₄) δ −112.94 (d, 1F), −115.23 (d, 1F).

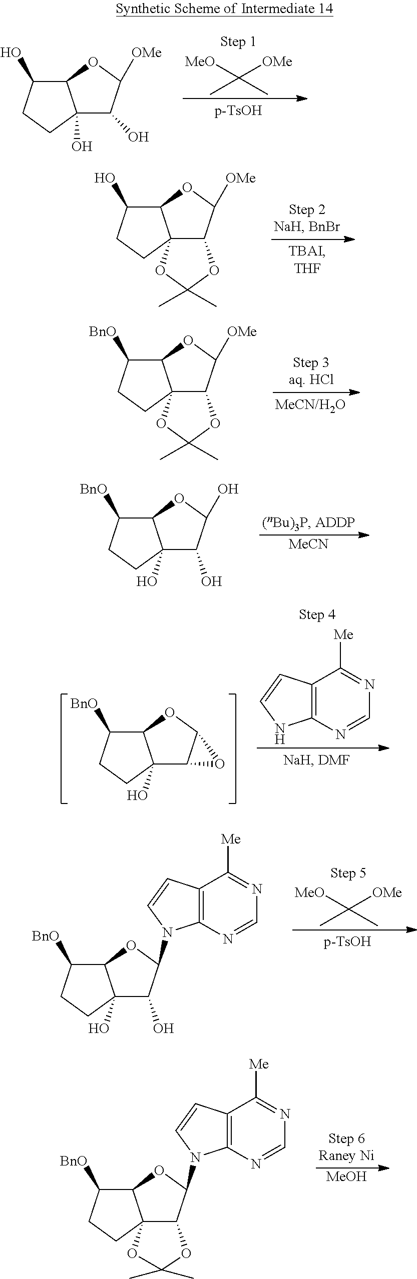

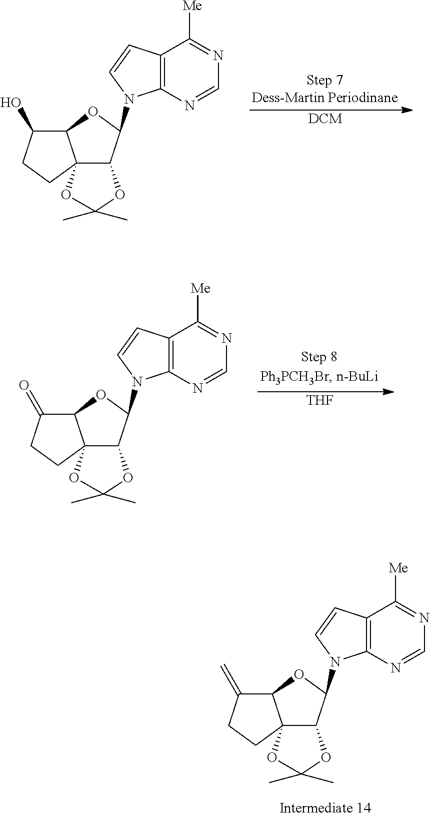

### Intermediate 14: 7-((3aR,4R,5aR,8aR)-2,2-dimethyl-6-methylenehexahydrocyclopenta[2,3]furo[3,4-d][1,3]dioxol-4-yl)-4-methyl-7H-pyrrolo[2,3-d]pyrimidine

Step 1: (3R,3aS,6R,6aR)-2-methoxyhexahydro-2H-cyclopenta[b]furan-3,3a,6-triol (2 g, 10 mmol) was co-evaporated with dry toluene (5 mL×3) and then re-dissolved in acetone (50 mL). To this solution was added 4-methylbenzenesulfonic acid (0.091 g, 0.53 mmol), followed by 2,2-dimethoxypropane (2.74 g, 26.3 mmol). The resulting mixture was stirred at ambient temperature for 1 h. The pH of the resulting solution was adjusted to 8 with saturated aqueous NaHCO₃ (50 mL) at 0° C. The resulting mixture was extracted with EtOAc (150 mL×3). The combined organic layers were washed with brine (100 mL), dried over anhydrous Na₂SO₄, filtered, and the filtrate was concentrated under reduced pressure. The residue was purified by column chromatography on silica (EtOAc/pet. ether) to afford (3aR,5aR,6R,8aR)-4-methoxy-2,2-dimethylhexahydrocyclopenta[2,3]furo[3,4-d][1,3]dioxol-6-ol. MS: 248.20 (M+NH₄). ¹H NMR (300 MHz, DMSO-d₆) δ 4.96 (s, 1H), 4.41 (d, J=5.1 Hz, 1H), 4.17 (s, 1H), 4.10 (d, J=6.0 Hz, 1H), 3.88-3.79 (m, 1H), 3.33 (s, 3H), 2.04-1.92 (m, 1H), 1.76-1.62 (m, 3H), 1.39 (s, 3H), 1.31 (s, 3H). The column was further eluted with 45-50% of EtOAc in petroleum ether to afford (3aR,5aR,6R,8aR)-4-methoxy-2,2-dimethylhexahydrocyclopenta[2,3]furo[3,4-d][1,3]dioxol-6-ol. MS: 248 (M+NH₄). ¹H NMR (300 MHz, DMSO-d₆) δ 4.92 (d, J=4.2 Hz, 1H), 4.72 (d, J=6.0 Hz, 1H), 4.35 (d, J=4.2 Hz, 1H), 4.00 (d, J=(5.4 Hz, 1H), 3.91-3.82 (m, 1H), 3.35 (s, 3H), 2.09-1.97 (m, 1H), 1.83-1.62 (m, 2H), 1.52-1.43 (m, 1H), 1.40 (s, 3H), 1.31 (s, 3H).

Step 2: To a mixture of sodium hydride (60% wt. dispersed in mineral oil, 0.88 g, 22 mmol) in anhydrous THF (20 mL) was added tetrabutylammonium iodide (0.67 g, 1.8 mmol) at ambient temperature under argon atmosphere. The mixture was cooled to 0° C., and a solution of (3aR,5aR,6R,8aR)-4-methoxy-2,2-dimethylhexahydrocyclopenta[2,3]furo[3,4-d][1,3]dioxol-6-ol (4.2 g, 18 mmol) in THF (15 mL) was added. The mixture was stirred for 0.5 h at ambient temperature. A solution of (bromomethyl)benzene (2.6 mL, 22 mmol) in THF (5 mL) was added to the mixture at 0° C. The resulting mixture was stirred at ambient temperature for 12 h. The reaction mixture was quenched with saturated aqueous NH₄Cl (100 mL) at 0° C. The resulting mixture was extracted with EtOAc (2×300 mL). The combined organic layers were washed with saturated aqueous NaHCO₃ (100 mL) and brine (100 mL). The organic layer was dried over anhydrous Na₂SO₄, filtered, and the filtrate was concentrated under reduced pressure. The crude product was purified by column chromatography on silica (0%-10% EtOAc/petroleum ether) to afford (3aR,4S,5aR,6R,8aR)-6-(benzyloxy)-4-methoxy-2,2-dimethylhexahydrocyclopenta[2,3]furo[3,4-d][1,3]dioxole. MS: 343 (M+Na). ¹H NMR (300 MHz, DMSO-d₆) δ 7.33-7.25 (m, 5H), 4.96-4.94 (m, 1H), 4.59 (d, J=11.7 Hz, 1H), 4.42 (d, J=11.7 Hz, 1H), 4.34 (d, J=6.0 Hz, 1H), 4.19-4.17 (m, 1H), 3.77-3.70 (m, 1H), 3.24 (s, 3H), 2.04-1.97 (m, 1H), 1.85-1.64 (m, 3H), 1.38 (s, 3H), 1.29 (s, 3H).

Step 3: To a solution of (3aR,4S,5aR,6R,8aR)-6-(benzyloxy)-4-methoxy-2,2-dimethylhexahydro cyclopenta[2,3]furo[3,4-d][1,3]dioxole (5.7 g, 18 mmol) in acetonitrile (150 mL) and water (100 mL) was added concentrated aq. hydrochloric acid (8.6 mL, 103 mmol) dropwise at ambient temperature. The reaction mixture was stirred at 90° C. for 1 h. The pH value of the resulting solution was adjusted to 7 with 1 M aq. NaOH at 0° C. The mixture was concentrated under reduced pressure. The residue was purified by column chromatography on silica (0-10% Methanol/DCM) to give (3R,3aS,6R,6aR)-6-(benzyloxy)hexahydro-2H-cyclopenta[b]furan-2,3,3a-triol. MS: 284 (M+NH₄).

Step 4: To a stirred mixture of (3R,3aS,6R,6aR)-6-(benzyloxy)hexahydro-2H-cyclopenta[b]furan-2,3,3a-triol (1.7 g, 6.4 mmol) in dry acetonitrile (100 mL) was added tributylphosphine (2.55 mL, 10 mmol) under argon atmosphere, followed by (E)-diazene-1,2-diylbis(piperidin-1-ylmethanone) (2.4 g, 9.6 mmol) at room temperature. The resulting mixture was stirred at room temperature for 30 min. The resulting epoxide containing solution was used directly without any further processing. A separate round bottom flask was charged with a solution of 4-methyl-7H-pyrrolo[2,3-d]pyrimidine (1.7 g, 13 mmol) in dry DMF (25 mL). To this was added sodium hydride (60 wt. % dispersed in mineral oil) (0.77 g, 19 mmol) at 0° C. under argon atmosphere. The suspension was stirred at room temperature for 30 min, and then it was transferred to the previous obtained epoxide containing solution by means of a syringe. The resulting mixture was stirred at room temperature for 1 h. The reaction was quenched by the addition of saturated aqueous ammonium chloride (100 mL) and extracted with EtOAc (3×100 mL). The combined organic layers were washed with brine (100 mL), dried over anhydrous sodium sulfate, and filtered. The filtrate was concentrated under reduced pressure. The residue was purified by reverse-phase column chromatography on C18 (0-95% 5 mM aq. NH₄HCO₃/ACN) to give (2R,3R,3aS,6R,6aR)-6-(benzyloxy)-2-(4-methyl-7H-pyrrolo[2,3-d]pyrimidin-7-yl)hexahydro-2H-cyclopenta[b]furan-3,3a-diol. MS: 382 (M+1). ¹H-NMR (300 MHz, DMSO-d₆) δ 8.69 (s, 1H), 7.74 (d, J=3.6 Hz, 1H), 7.36-7.25 (m, 5H), 6.73 (d, J=3.9 Hz, 1H), 6.16 (d, J=8.4 Hz, 1H), 5.41 (d, J=6.9 Hz, 1H), 5.24 (s, 1H), 4.55-4.50 (m, 2H), 4.27-4.19 (m, 2H), 3.92-3.86 (m, 1H), 2.67 (s, 3H), 2.02-1.98 (m, 3H), 1.60-1.52 (m, 1H).

Step 5: To a mixture of (2R,3R,3aS,6R,6aR)-6-(benzyloxy)-2-(4-methyl-7H-pyrrolo[2,3-d]pyrimidin-7-yl)hexahydro-2H-cyclopenta[b]furan-3,3a-diol (2.4 g, 6.3 mmol) in 2,2-dimethoxypropane (50 mL) under argon atmosphere was added 4-methylbenzenesulfonic acid (0.11 g, 0.63 mmol) at ambient temperature. The mixture was stirred at 70° C. for 48 h. The mixture was quenched with saturated aqueous NaHCO₃ (50 mL), and then extracted with DCM (100 mL×3). The combined organic layers were dried over anhydrous Na₂SO₄, filtered, and the filtrate was concentrated under reduced pressure. The residue was purified by column chromatography on silica (0-60% EtOAc/Petroleum ether) to give 7-((3aR,4R,5aR,6R,8aR)-6-(benzyloxy)-2,2-dimethylhexahydrocyclopenta[2,3]furo[3,4-d][1,3]dioxol-4-yl)-4-methyl-7H-pyrrolo[2,3-d]pyrimidine. MS: 422 (M+1). ¹H-NMR (400 MHz, DMSO-d6) δ 8.71 (s, 1H), 7.79 (d, J=4.0 Hz, 1H), 7.33-7.23 (m, 5H), 6.81 (d, J=3.6 Hz, 1H), 6.32 (d, J=4.4 Hz, 1H), 5.15 (d, J=4.8 Hz, 1H), 4.51 (q, J=12.0 Hz, 2H), 4.42 (d, J=4.4 Hz, 1H), 3.93-3.87 (m, 1H), 2.67 (s, 3H), 2.47-2.41 (m, 1H), 2.03-1.99 (m, 1H), 1.95-1.83 (m, 2H), 1.55 (s, 3H), 1.36 (s, 3H).

Step 6: To a solution of 7-((3aR,4R,5aR,6R,8aR)-6-(benzyloxy)-2,2-dimethylhexahydrocyclopenta[2,3]furo[3,4-d][1,3]dioxol-4-yl)-4-methyl-7H-pyrrolo[2,3-d]pyrimidine (1.2 g, 2.9 mmol) in anhydrous MeOH (35 mL) under argon atmosphere was added wet Raney Ni (8 g, 50 wt. % in water) at ambient temperature. The resulting mixture was stirred at 60° C. for 5 h. The resulting mixture was filtered, and the filtrate was concentrated under reduced pressure. The product was purified by column chromatography on silica (0-100% EtOAc/petroleum ether) to give (3aR,4R,5aR,6R,8aR)-2,2-dimethyl-4-(4-methyl-7H-pyrrolo[2,3-d]pyrimidin-7-yl)hexahydrocyclopenta[2,3]furo[3,4-d][1,3]dioxol-6-ol. MS: 332 (M+1). ¹H-NMR (400 MHz, DMSO-d₆) δ 8.70 (s, 1H), 7.83 (d, J=4.0 Hz, 1H), 6.83 (d, J=4.0 Hz, 1H), 6.29 (d, J=4.4 Hz, 1H), 5.10 (d, J=4.8 Hz, 1H), 4.92 (d, J=6.0 Hz, 1H), 4.15 (d, J=4.8 Hz, 1H), 3.98-3.94 (m, 1H), 2.68 (s, 3H), 2.43-2.35 (m, 1H), 1.90-1.84 (m, 3H), 1.55 (s, 3H), 1.36 (s, 3H).

Step 7: To a mixture of (3aR,4R,5aR,6R,8aR)-2,2-dimethyl-4-(4-methyl-7H-pyrrolo[2,3-d]pyrimidin-7-yl)hexahydrocyclopenta[2,3]furo[3,4-d][1,3]dioxol-6-ol (2.0 g, 6.04 mmol) in DCM (60 mL) was added Dess-Martin Periodinane (4.6 g, 11 mmol) at 25° C. under argon atmosphere. The resulting mixture was stirred at 25° C. for 1.5 h. The mixture was quenched by the addition of saturated aqueous NaHCO₃ (150 mL) and extracted with EtOAc (3×250 mL). The combined organic layers were dried over anhydrous Na₂SO₄, filtered, and the filtrate was concentrated under reduced pressure. The residue was purified by column chromatography on silica (0-100% EtOAc/Petroleum ether to give (3aR,4R,5aS,8aS)-2,2-dimethyl-4- (4-methyl-7H-pyrrolo[2,3-d]pyrimidin-7-yl)tetrahydrocyclopenta[2,3]furo[3,4-d][1,3]dioxol-6(5aH)-one. MS: 330 (M+1). ¹H-NMR (400 MHz, DMSO-d₆) δ 8.67 (s, 1H), 7.63 (d, J=3.6 Hz, 1H), 6.75 (d, J=3.6 Hz, 1H), 6.36 (d, J=2.4 Hz, 1H), 5.51 (d, J=2.8 Hz, 1H), 4.56 (s, 1H), 2.96-2.82 (m, 1H), 2.78-2.59 (m, 5H), 2.42-2.34 (m, 1H), 1.57 (s, 3H), 1.46 (s, 3H).

Step 8: To a mixture of bromo(methyl)triphenylphosphorane (5.8, 16 mmol) in THF (30 mL) was added n-butyllithium (2.5 M in hexane, 6 mL, 15 mmol) at −10° C. under argon atmosphere. The resulting mixture was stirred at −10° C. for 0.5 h. To this was added dropwise a solution of (3aR,4R,5aS,8aS)-2,2-dimethyl-4-(4-methyl-7H-pyrrolo[2,3-d]pyrimidin-7-yl)tetrahydrocyclopenta[2,3]furo[3,4-d][1,3]dioxol-6(5aH)-one (1.9 g, 5.8 mmol) in THF (30 mL) at −10° C. The resulting mixture was stirred at −10° C. for 1 h. The mixture was quenched by the addition of saturated aqueous NH₄Cl (150 mL) then extracted with DCM (3×200 mL). The combined organic layers were dried over anhydrous Na₂SO₄, filtered, and the filtrate was concentrated under reduced pressure. The residue was purified by column chromatography on silica (0-60% EtOAc/Petroleum ether) to give 7-((3aR,4R,5aR,8aR)-2,2-dimethyl-6-methylenehexahydrocyclopenta[2,3]furo[3,4-d][1,3]dioxol-4-yl)-4-methyl-7H-pyrrolo[2,3-d]pyrimidine. 328 (M+1). ¹H-NMR (400 MHz, DMSO-d₆) δ 8.70 (s, 1H), 7.68 (d, J=3.6 Hz, 1H), 6.80 (d, J=3.6 Hz, 1H), 6.32 (d, J=4.0 Hz, 1H), 5.27 (d, J=4.0 Hz, 1H), 5.13-5.11 (m, 2H), 4.61 (s, 1H), 2.67 (s, 3H), 2.61-2.40 (m, 3H), 2.05-1.95 (m, 1H), 1.56 (s, 3H), 1.38 (s, 3H).

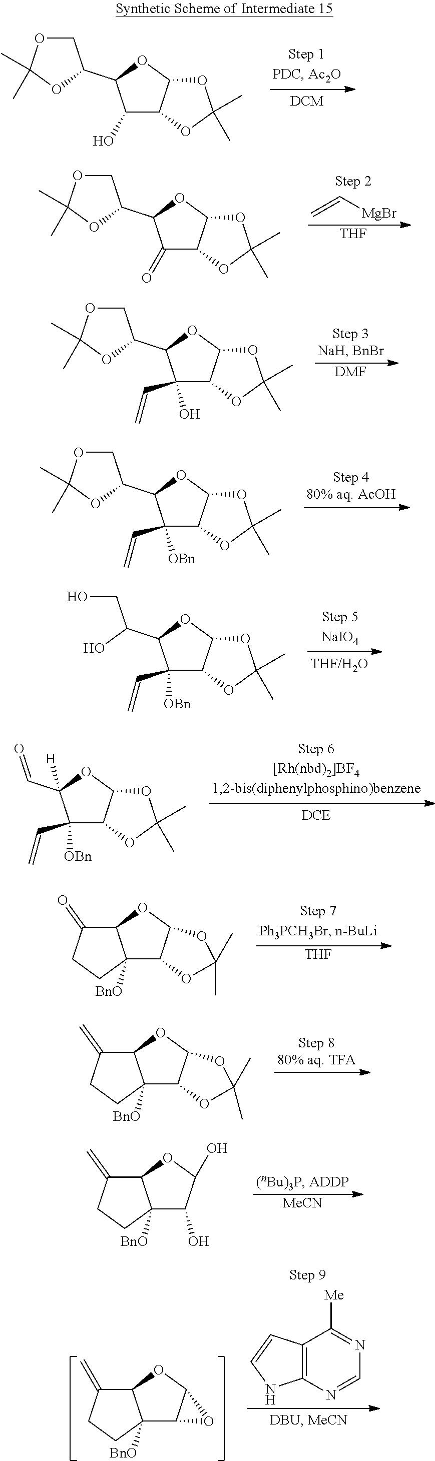

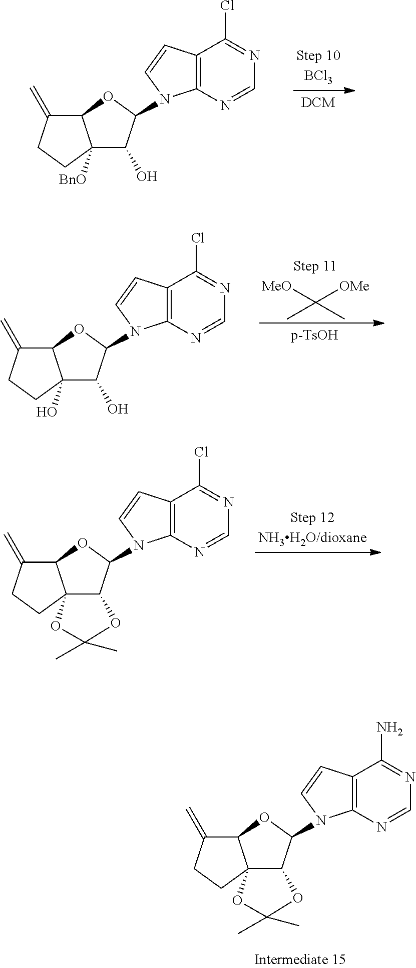

### Intermediate 15: 7-((3aR,4R,5aR,8aR)-2,2-dimethyl-6-methylenehexahydrocyclopenta[2,3]furo[3,4-d][1,3]dioxol-4-yl)-7H-pyrrolo[2,3-d]pyrimidin-4-amine

Step 1: To a stirred solution of (3aR,5S,6R,6aR)-5-((R)-2,2-dimethyl-1,3-dioxolan-4-yl)-2,2-dimethyltetrahydrofuro[2,3-d][1,3]dioxol-6-ol (200 g, 768 mmol) in DCM (1000 mL) was added pyridinium dichromate (170 g, 760 mmol) and acetic anhydride (220 mL, 2.3 mol) at room temperature. The resulting mixture was stirred at 40° C. for 2 h. The mixture was concentrated under reduced pressure, and the residue was purified by column chromatography on silica (10-40% EtOAc/petroleum ether to give (3aR,5R,6aS)-5-((R)-2,2-dimethyl-1,3-dioxolan-4-yl)-2,2-dimethyldihydrofuro[2,3-d][1,3]dioxol-6(3aH)-one. MS: 276 (M+NH₄). ¹H NMR (400 MHz, Chloroform-d) δ 6.11 (d, J=4.5 Hz, 1H), 4.42-4.31 (m, 2H), 4.10-3.95 (m, 2H), 3.44-3.39 (m, 1H), 1.46-1.41 (m, 6H), 1.31 (s, 6H).

Step 2: To a stirred solution of (3aR,5R,6aS)-5-((R)-2,2-dimethyl-1,3-dioxolan-4-yl)-2,2-dimethyldihydrofuro[2,3-d][1,3]dioxol-6(3aH)-one (160 g, 600 mmol) in THF (1500 mL) was added vinyl magnesium bromide (1 M in THF, 900 mL, 900 mmol) at −78° C. under argon atmosphere. The resulting mixture was stirred at room temperature for 2 h. The mixture was quenched with sat. aqueous NH₄Cl (500 mL). The mixture was extracted with EtOAc (3×1000 mL). The combined organic layers were washed with brine (1000 mL), dried over anhydrous Na₂SO₄, and filtered. The filtrate was concentrated under reduced pressure, and the residue was purified column chromatography on silica (1-15% EtOAc/petroleum ether) to give (3aR,5R,6R,6aR)-5-((R)-2,2-dimethyl-1,3-dioxolan-4-yl)-2,2-dimethyl-6-vinyltetrahydrofuro[2,3-d][1,3]dioxol-6-ol. MS: 304 (M+NH₄). ¹H NMR (400 MHz, DMSO-d₆) δ 5.81 (d, J=3.6 Hz, 1H), 5.77-5.67 (m, 1H), 5.40-5.36 (m, 1H), 5.29-5.26 (m, 1H), 5.24-5.19 (m, 1H), 4.20-4.16 (m, 1H), 4.08-4.06 (m, 1H), 4.02-3.96 (m, 1H), 3.79-3.69 (m, 2H), 1.49 (s, 3H), 1.36-1.20 (m, 9H).

Step 3: Sodium hydride (60 wt. % dispersed in mineral oil, 28 g, 700 mmol) was suspended in anhydrous DMF (1000 mL) under argon atmosphere, and the mixture was cooled to 0° C. A solution of (3aR,5R,6R,6aR)-5-((R)-2,2-dimethyl-1,3-dioxolan-4-yl)-2,2-dimethyl-6-vinyltetrahydrofuro[2,3-d][1,3]dioxol-6-ol (133 g, 465 mmol) in anhydrous DMF (300 mL) was added dropwise over a period of 45 min. The mixture was stirred at 50° C. for 1 h then cooled to 0° C. Bromomethyl benzene (160 g, 930 mmol) was added dropwise, and the reaction mixture was stirred at room temperature for 3 h. The reaction mixture was quenched with sat. aqueous NH₄Cl (1300 mL) and extracted with EtOAc (3×1000 mL). The combined organic layers were washed with brine (2000 mL), dried over anhydrous Na₂SO₄, filtered, and the filtrate was concentrated under reduced pressure. The residue was purified by column chromatography on silica (1-20% EtOAc/petroleum ether) to give (3aR,5R,6R,6aR)-6-(benzyloxy)-5-((R)-2,2-dimethyl-1,3-dioxolan-4-yl)-2,2-dimethyl-6-vinyltetrahydrofuro[2,3-d][1,3]dioxole. MS: 394 (M+NH₄). ¹H NMR (300 MHz, DMSO-d₆) δ 7.40-7.24 (m, 5H), 5.89 (d, J=3.3 Hz, 1H), 5.86-5.76 (m, 1H), 5.49-5.36 (m, 2H), 4.79 (d, J=3.6 Hz, 1H), 4.55-4.46 (m, 2H), 4.14-4.02 (m, 2H), 3.90-3.85 (m, 1H), 3.74-3.69 (m, 1H), 1.50 (s, 3H), 1.30 (s, 3H), 1.27 (s, 3H), 1.24 (s, 3H).

Step 4: (3aR,5R,6R,6aR)-6-(benzyloxy)-5-((R)-2,2-dimethyl-1,3-dioxolan-4-yl)-2,2-dimethyl-6-vinyltetrahydrofuro[2,3-d][1,3]dioxole (130 g, 350 mmol) was dissolved in 80% aq. acetic acid (900 mL) and the reaction mixture was stirred at 50° C. for 2 h. The reaction mixture was concentrated under reduced pressure and co-evaporated with toluene (2×300 mL). The residue was partitioned between EtOAc (1000 mL) and sat. aqueous NaHCO₃ (900 mL). The organic phase was combined and concentrated under reduced pressure to give 1-((3aR,5R,6R,6aR)-6-(benzyloxy)-2,2-dimethyl-6-vinyltetrahydrofuro[2,3-d][1,3]dioxol-5-yl)ethane-1,2-diol. The product was used without further purification. MS: 354 (M+NH₄).

Step 5: To a stirred solution of 1-((3aR,5R,6R,6aR)-6-(benzyloxy)-2,2-dimethyl-6-vinyl tetrahydrofuro[2,3-d][1,3]dioxol-5-yl)ethane-1,2-diol (60 g, 180 mmol) in THF (100 mL) was added a solution of sodium periodate (60 g, 270 mmol) in water (100 mL). The reaction was stirred at room temperature for 1 h. Water (200 mL) was added and the resulting mixture was extracted with DCM (3×300 mL). The combined organic layers were washed with brine (800 mL), dried over anhydrous sodium sulfate, and filtered. The filtrate was concentrated under reduced pressure, and the residue was purified by column chromatography on silica (10-30% EtOAc/petroleum ether to give (3aR,5S,6R,6aR)-6-(benzyloxy)-2,2-dimethyl-6-vinyltetrahydrofuro[2,3-d][1,3]dioxole-5-carbaldehyde. MS: 322 (M+NH₄). ¹H NMR (400 MHz, Chloroform-d) δ 9.58 (s, 1H), 7.42-7.28 (m, 5H), 6.01-5.97 (m, 1H), 5.81-5.74 (m, 1H), 5.55-5.41 (m, 2H), 4.75-4.62 (m, 4H), 1.62 (s, 3H), 1.41 (s, 3H).

Step 6: Bis(norbomadiene) rhodium (I) tetrafluoroborate (0.74 g, 2.0 mmol) and 1,2-bis(diphenylphosphino)benzene (1.1 g, 2.4 mmol) were suspended in DCE (70 mL). The mixture was stirred at room temperature under an atmosphere of argon for 10 min. Then hydrogen was bubbled through the solution for 10 min, followed by flushing again with argon for 20 min. (3aR,5S,6R,6aR)-6-(benzyloxy)-2,2-dimethyl-6-vinyltetrahydrofuro[2,3-d][1,3]dioxole-5-carbaldehyde (6 g, 20 mmol) in DCE (120 mL) was added dropwise to the above solution, and the mixture was stirred for 20 h at 75° C. The reaction mixture was concentrated under reduced pressure, and the residue was purified by column chromatography on silica (1-15% EtOAc/petroleum ether) to give (3aR,4aS,7aS,7bR)-7a-(benzyloxy)-2,2-dimethyltetrahydro-3aH-cyclopenta[4,5]furo[2,3-d][1,3]dioxol-5(4aH)-one. MS: 322 (M+NH₄). ¹H NMR (300 MHz, Chloroform-d) δ 7.40-7.31 (m, 5H), 5.96 (d, J=3.6 Hz, 1H), 4.77 (d, J=10.8 Hz, 1H), 4.67-4.61 (m, 2H), 4.19 (s, 1H), 2.58-2.43 (m, 3H), 1.82-1.68 (m, 1H), 1.66 (s, 3H), 1.42 (s, 3H).

Step 7: To a stirred mixture of bromo(methyl)triphenylphosphorane (28.3 g, 79 mmol) in THF (109 mL) was added n-butyllithium (2.5 M in hexane, 28 mL, 71 mmol) dropwise at −60° C. under argon atmosphere. The resulting mixture was stirred at room temperature for 0.5 h. (3aR,4aS,7aS,7bR)-7a-(benzyloxy)-2,2-dimethyltetrahydro-3aH-cyclopenta[4,5]furo[2,3-d][1,3]dioxol-5(4aH)-one (8.6 g, 28.3 mmol) in THF (110 mL) was then added dropwise to the above solution by syringe at −60° C. The reaction mixture was stirred at room temperature for 2 h. The mixture was quenched with saturated aqueous brine (200 mL) at 0° C. The mixture was extracted with EtOAc (3×300 mL). The combined organic layers were dried over anhydrous sodium sulfate and filtered. The filtrate was concentrated under reduced pressure. The residue was purified by column chromatography on silica (0-30% EtOAc/petroleum ether) to afford (3aR,4aR,7aR,7bR)-7a-(benzyloxy)-2,2-dimethyl-5-methylenehexahydro-5H-cyclopenta[4,5]furo[2,3-d][1,3]dioxole. ¹H-NMR (400 MHz, DMSO-d₆) δ 7.34-7.25 (m, 5H), 5.87 (d, J=4.0 Hz, 1H), 5.23-5.22 (m, 1H), 5.10-5.09 (m, 1H), 4.68 (d, J=3.6 Hz, 1H), 4.59 (d, J=11.2 Hz, 1H), 4.51 (d, J=11.2 Hz, 1H), 4.44 (s, 1H), 2.49-2.39 (m, 2H), 2.22-2.16 (m, 1H), 1.63-1.55 (m, 1H), 1.51 (s, 3H), 1.32 (s, 3H).

Step 8: To (3aR,4aR,7aR,7bR)-7a-(benzyloxy)-2,2-dimethyl-5-methylenehexahydro-5H-cyclopenta[4,5]furo[2,3-d][1,3]dioxole (6.8 g, 22 mmol) was added a solution of TFA (45 mL) in water (11 mL) at 0° C. The resulting mixture was stirred at room temperature for 0.25 h. The mixture was neutralized with 2 M aq. NaOH then extracted with EtOAc (4×200 mL). The combined organic layers were dried over anhydrous sodium sulfate and filtered. The filtrate was concentrated under reduced pressure. The residue was purified by silica gel column chromatography (0-70% EtOAc in petroleum ether) to afford (3R,3aS,6aR)-3a-(benzyloxy)-6-methylenehexahydro-2H-cyclopenta[b]furan-2,3-diol as a mixture of two diastereomers at the anomeric center in 5:4 ratio. ¹H-NMR (400 MHz, DMSO-d₆) δ 7.37-7.24 (m, 5H), 6.51-6.06 (m, 1H), 5.25-4.87 (m, 4H), 4.68-4.36 (m, 3H), 3.87-3.76 (m, 1H), 2.57-2.33 (m, 2H), 2.10-1.72 (m, 2H).

Step 9: To a stirred solution of (3R,3aS,6aR)-3a-(benzyloxy)-6-methylenehexahydro-2H-cyclopenta[b]furan-2,3-diol (5.0 g, 19 mmol) in dry acetonitrile (63 mL) under the atmosphere of argon was added dropwise (E)-diazene-1,2-diylbis(piperidin-1-ylmethanone) (7.2 g, 29 mmol) in acetonitrile (63 mL) via syringe over 0.5 min at room temperature. Tributylphosphine (7.6 mL, 31 mmol) was added via syringe over 5 min at room temperature. The reaction solution was stirred at room temperature for about 5 min. The reaction mixture was stirred at 46° C. for 3 h. The resultant epoxide mixture was used directly. In parallel, a separate round bottom flask was charged with a solution of 4-chloro-7H-pyrrolo[2,3-d]pyrimidine (5.6 g, 36 mmol) in dry acetonitrile (30 mL) and DBU (5.2 mL, 34 mmol) at room temperature under an atmosphere of argon. The resulting mixture was stirred at room temperature for 30 min. Then the DBU containing solution was transferred to the above mixture containing the epoxide intermediate by means of a syringe at room temperature under argon atmosphere. The resulting mixture was stirred at 46° C. for 2 h, and then concentrated under reduced pressure. The residue was purified by column chromatography on silica (0-30% EtOAc/petroleum ether) to afford (2R,3R,3aS,6aR)-3a-(benzyloxy)-2-(4-chloro-7H-pyrrolo[2,3-d]pyrimidin-7-yl)-6-methylenehexahydro-2H-cyclopenta[b]furan-3-ol. MS: 398 (M+1). ¹H-NMR (400 MHz, DMSO-d₆) δ 8.71 (s, 1H), 7.96 (d, J=4.0 Hz, 1H), 7.45-7.28 (m, 5H), 6.80 (d, J=3.6 Hz, 1H), 6.30 (d, J=8.0 Hz, 1H), 5.83 (d, J=6.8 Hz, 1H), 5.14 (d, J=16.0 Hz, 2H), 4.92 (d, J=12.0 Hz, 1H), 4.71 (d, J=11.6 Hz, 1H), 4.67 (s, 1H), 4.61-4.58 (m, 1H), 2.84-2.78 (m, 1H), 2.56-2.51 (m, 1H), 2.19-2.13 (m, 1H), 2.09-2.04 (m, 1H).

Step 10: To a solution of (2R,3R,3aS,6aR)-3a-(benzyloxy)-2-(4-chloro-7H-pyrrolo[2,3-d]pyrimidin-7-yl)-6-methylenehexahydro-2H-cyclopenta[b]furan-3-ol (690 mg, 1.7 mmol) in DCM (10 mL) was added dropwise trichloroborane (1 M in DCM, 3.5 mL, 3.5 mmol) at −78° C. under argon atmosphere. The resulting solution was stirred at −78° C. for 3 h. The reaction mixture was quenched by the addition of TEA (1.0 mL, 7.0 mmol) then stirred at −78° C. for 0.5 h. The reaction solution was poured into saturated aqueous NaHCO₃ (150 mL) at 0° C. with vigorous stirring. The mixture was extracted by EtOAc (3×200 mL). The organic layers were dried over anhydrous sodium sulfate and filtered. The filtrate was concentrated under reduced pressure. The crude residue was purified by column chromatography on silica (0-10% MeOH/DCM) to afford (2R,3R,3aS,6aR)-2-(4-chloro-7H-pyrrolo[2,3-d]pyrimidin-7-yl)-6-methylenehexahydro-2H-cyclopenta[b]furan-3,3a-diol. MS: 308 (M+1). ¹H-NMR (400 MHz, DMSO-d₆) δ 8.72 (s, 1H), 7.95 (d, J=4.0 Hz, 1H), 6.78 (d, J=4.0 Hz, 1H), 6.21 (d, J=8.0 Hz, 1H), 5.52 (d, J=7.2 Hz, 1H), 5.38 (s, 1H), 5.12-5.07 (m, 2H), 4.44-4.34 (m, 2H), 2.78-2.69 (m, 1H), 2.51-2.42 (m, 1H), 2.08-2.03 (m, 1H), 1.72-1.64 (m, 1H).

Step 11: To a mixture of (2R,3R,3aS,6aR)-2-(4-chloro-7H-pyrrolo[2,3-d]pyrimidin-7-yl)-6-methylenehexahydro-2H-cyclopenta[b]furan-3,3a-diol (720 mg, 2.3 mmol) in 2,2-dimethoxypropane (2 mL) was added 4-methylbenzenesulfonic acid (40 mg, 0.23 mmol) at ambient temperature. The mixture was stirred for 16 h at ambient temperature. The reaction mixture was quenched with NaHCO₃ (200 mg) at ambient temperature. The reaction mixture was concentrated under reduced pressure. The residue was purified by column chromatography on silica (0-30% EtOAc/petroleum ether) to give 4-chloro-7-((3aR,4R,5aR,8aR)-2,2-dimethyl-6-methylenehexahydrocyclopenta[2,3]furo[3,4-d][1,3]dioxol-4-yl)-7H-pyrrolo[2,3-d]pyrimidine. MS: 348 (M+1). ¹H-NMR (400 MHz, DMSO-d₆) δ 8.72 (s, 1H), 7.87 (d, J=4.0 Hz, 1H), 6.79 (d, J=4.0 Hz, 1H), 6.36 (d, J=3.6 Hz, 1H), 5.30 (d, J=3.6 Hz, 1H), 5.15-5.14 (m, 2H), 4.68 (s, 1H), 2.58-2.41 (m, 3H), 2.04-1.93 (m, 1H), 1.57 (s, 3H), 1.39 (s, 3H).

Step 12: To 4-chloro-7-((3aR,4R,5aR,8aR)-2,2-dimethyl-6-methylenehexahydrocyclopenta[2,3]furo[3,4-d][1,3]dioxol-4-yl)-7H-pyrrolo[2,3-d]pyrimidine (2.7 g, 7.76 mmol) was added 1,4-dioxane (18 mL) and concentrated aqueous ammonia (28 wt. %, 18 mL) at room temperature. The reaction container was sealed and stirred at 90° C. for 16 h. The mixture was concentrated under reduced pressure. The residue was purified by column chromatography on silica (0-10% MeOH/DCM) to give 7-((3aR,4R,5aR,8aR)-2,2-dimethyl-6-methylenehexahydrocyclopenta[2,3]furo[3,4-d][1,3]dioxol-4-yl)-7H-pyrrolo[2,3-d]pyrimidin-4-amine. MS: 329 (M+1). ¹H-NMR (400 MHz, DMSO-d₆) δ 8.09 (s, 1H), 7.29 (d, J=3.6 Hz, 1H), 7.10 (s, 2H), 6.64 (d, J=3.6 Hz, 1H), 6.20 (d, J=4.4 Hz, 1H), 5.19 (d, J=4.0 Hz, 1H), 5.13-5.11 (m, 2H), 4.55 (s, 1H), 2.63-2.42 (m, 3H), 2.01-1.96 (m, 1H), 1.55 (s, 3H), 1.38 (s, 3H).

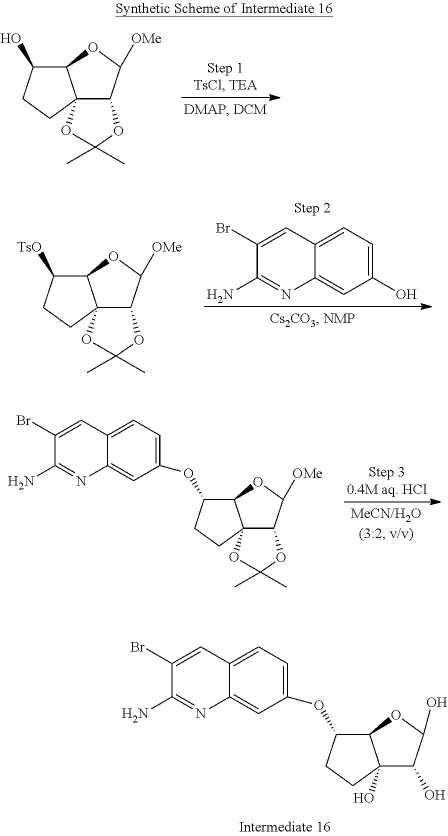

### Intermediate 16: (3R,3aS,6S,6aR)-6-((2-amino-3-bromoquinolin-7-yl)oxy)hexahydro-3aH-cyclopenta[b]furan-2,3,3a-triol

Step 1: To a solution of (3aR,5aR,6R,8aR)-4-methoxy-2,2-dimethylhexahydrocyclopenta[2,3]furo[3,4-d][1,3]dioxol-6-ol (5.0 g, 22 mmol) in DCM (40 mL) was added 4-dimethylaminopyridine (2.9 g, 24 mmol) at room temperature. To the mixture was added dropwise triethylamine (2.4 g, 24 mmol) followed by p-toluenesulfonyl chloride (6.2 g, 33 mmol). The reaction mixture was stirred at 25° C. for 16 h. The resulting mixture was quenched with saturated aqueous NH₄Cl (100 mL) and extracted with DCM (100 mL×3). The combined organic layers were washed with brine (100 mL) and dried over anhydrous sodium sulfate. After filtration, the filtrate was concentrated under reduced pressure. The residue was purified by column chromatography on silica (0-27% ethyl acetate/petroleum ether) to give (3aR,5aR,6R,8aR)-4-methoxy-2,2-dimethylhexahydrocyclopenta[2,3]furo[3,4-d][1,3]dioxol-6-yl 4-methylbenzenesulfonate. MS: 402 (M+NH₄). ¹H-NMR (400 MHz, DMSO-d₆) δ 7.84-7.82 (m, 2H), 7.52-7.49 (m, 2H), 4.87 (d, J=4.0 Hz, 1H), 4.77-4.72 (m, 1H), 4.40 (d, J=4.0 Hz, 1H), 3.93 (d, J=5.2 Hz, 1H), 3.24 (s, 3H), 2.44 (s, 3H), 2.13-2.08 (m, 1H), 1.91-1.86 (m, 1H), 1.78-1.57 (m, 2H), 1.36 (s, 3H), 1.28 (s, 3H).

Step 2: A mixture of 2-amino-3-bromoquinolin-7-ol (2.0 g, 8.2 mmol) and (3aR,5aR,6R,8aR)-4-methoxy-2,2-dimethylhexahydrocyclopenta[2,3]furo[3,4-d][1,3]dioxol-6-yl 4-methylbenzenesulfonate (3.0 g, 7.8 mmol) was co-evaporated with dry toluene (10 mL each, three times) and re-dissolved in NMP (10 mL). To this solution was added cesium carbonate (7.6 g, 23 mmol) at ambient temperature. The resulting mixture was stirred at 90° C. for 1.5 h. The reaction mixture was filtered, and the filtrate was purified by reversed-phase column chromatography on C18 (0-95% 5 mM aq. NH₄HCO₃/MeCN) to give 3-bromo-7-(((3aR,5aR,6S,8aR)-4-methoxy-2,2-dimethylhexahydrocyclopenta[2,3]furo[3,4-d][1,3]dioxol-6-yl)oxy)quinolin-2-amine. MS: 451/453 (M+1/M+3). ¹H-NMR (400 MHz, DMSO-d₆) δ 8.30 (s, 1H), 7.60 (d, J=8.4 Hz, 1H), 6.91-6.86 (m, 2H), 6.62 (s, 2H), 4.95 (d, J=4.4 Hz, 1H), 4.66 (d, J=4.0 Hz, 1H), 4.54 (d, J=4.4 Hz, 1H), 4.19-4.18 (m, 1H), 3.37 (s, 3H), 2.26-1.98 (m, 4H), 1.36 (s, 3H), 1.28 (s, 3H).

Step 3: 3-bromo-7-(((3aR,5aR,6S,8aR)-4-methoxy-2,2-dimethylhexahydrocyclopenta[2,3]furo[3,4-d][1,3]dioxol-6-yl)oxy)quinolin-2-amine (4.9 g, 11 mmol) was dissolved in 0.4 M aq. HCl in MeCN/H₂O (3:2, v/v) (120 mL) at 0° C. The resulting mixture was stirred at 90° C. for 3 h in a sealed tube. The reaction mixture was cooled to 0° C. The pH value of the solution was adjusted to 7˜8 with 2 M aq. NaOH. The resulting mixture was concentrated under reduced pressure, and the residue was purified by reverse-phase column chromatograph on AQ-C18 (0-95% 5 mM aq. NH₄HCO₃/MeCN) to give (3R,3aS,6S,6aR)-6-((2-amino-3-bromoquinolin-7-yl)oxy)hexahydro-3aH-cyclopenta[b]furan-2,3,3a-triol. MS: 397/399 (M+1/M+3). ¹H NMR (400 MHz, Methanol-d₄) δ 8.22-8.21 (m, 1H), 7.57-7.54 (m, 1H), 7.12-7.03 (m, 1H), 6.98-6.94 (m, 1H), 5.35-5.20 (m, 1H), 4.94-4.64 (m, 1H), 4.36-4.18 (m, 1H), 3.80-3.62 (m, 1H), 2.36-2.02 (m, 4H).

Intermediates 17-21: Intermediates 17-21 in Table 3 were synthesized using the protocol described in intermediate 16 (Synthetic Scheme of Intermediate 16) making the appropriate substitution for the 2-amino-3-bromoquinolin-7-ol in step 2. The substituted starting material was commercially acquired, synthesized as reported above, or synthesized through known routes reported in the literature.

TABLE 3

| Intermediate | Structure | Name | MS |
|---|---|---|---|
| 17 | 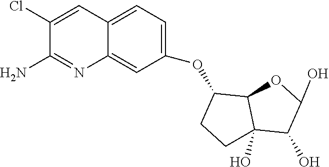 | (3R,3aS,6S,6aR)-6-[(2-amino-3- chloroquinolin-7-yl)oxy]hexahydro- 3aH-cyclopenta[b]furan-2,3,3a-triol | 353 (M + 1) |
| 18 | 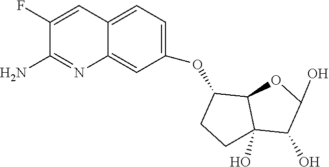 | (3R,3aS,6S,6aR)-6-[(2-amino-3- fluoroquinolin-7-yl)oxy]hexahydro- 3aH-cyclopenta[b]furan-2,3,3a-triol | 337 (M + 1) |
| 19 | 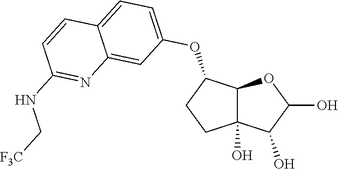 | (3R,3aS,6S,6aR)-6-((2-((2,2,2- trifluoroethyl)amino)quinolin-7- yl)oxy)hexahydro-3aH- cyclopenta[b]furan-2,3,3a-triol | 401 (M + 1) |
| 20 | 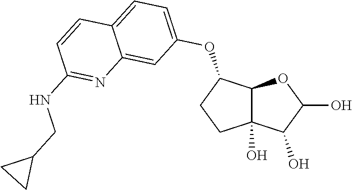 | (3R,3aS,6S,6aR)-6-((2- ((cyclopropylmethyl)amino)quinolin- 7-yl)oxy)hexahydro-3aH- cyclopenta[b]furan-2,3,3a-triol | 373 (M + 1) |
| 21 | 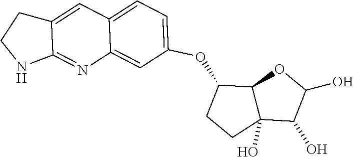 | (3R,3aS,6S,6aR)-6-((2,3-dihydro- 1H-pyrrolo[2,3-b]quinolin-7- yl)oxy)hexahydro-3aH- cyclopenta[b]furan-2,3,3a-triol | 345 (M + 1) |

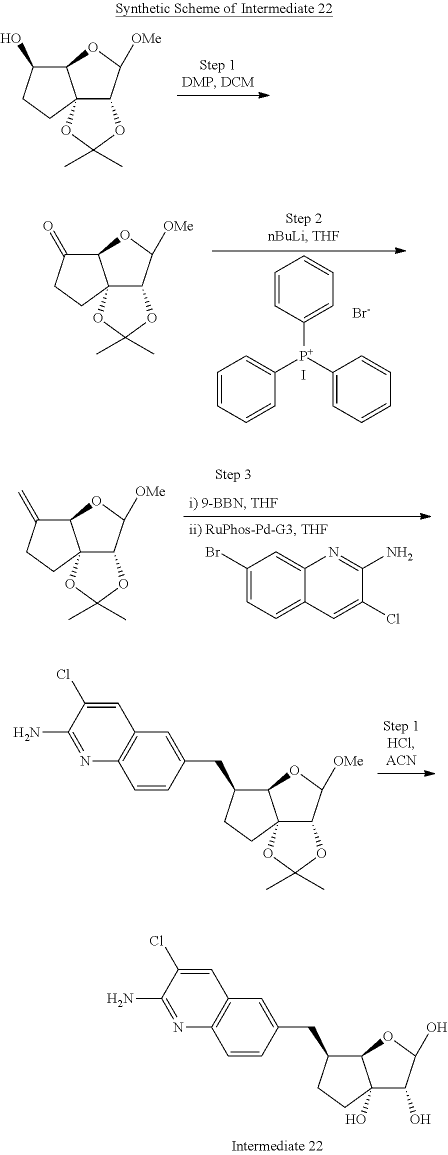

### Intermediate 22: (3R,3aS,6S,6aR)-6-[(2-amino-3-chloroquinolin-7-yl)methyl]hexahydro-3aH-cyclopenta[b]furan-2,3,3a-triol

Step 1: To a solution of (3aR,5aR,6R,8aR)-4-methoxy-2,2-dimethylhexahydrocyclopenta[2,3]furo[3,4-d][1,3]dioxol-6-ol (2.0 g, 8.7 mmol) in anhydrous DCM (43 mL) at 0° C. under nitrogen atmosphere was added DMP (4.4 g, 10 mmol) in one portion. The mixture was stirred at room temperature overnight. The mixture was diluted with DCM (40 mL) and treated with saturated aqueous sodium bicarbonate (80 mL) and sodium thiosulfate (10 g, 63 mmol). The resulting mixture was stirred for 10 minutes at room temperature. The organic layer was separated, and the aqueous phase was extracted with DCM (40 mL×3). The combined organic layers were washed with brine (80 mL), dried with anhydrous sodium sulfate, filtered, and concentrated under reduced pressure. The residue was purified by column chromatography on silica (0-60% EtOAc/hexanes) to afford (3aR,5aS,8aS)-4-methoxy-2,2-dimethyltetrahydrocyclopenta[2,3]furo[3,4-d][1,3]dioxol-6(5aH)-one. ¹H NMR (600 MHz, DMSO-d₆) δ 4.97 (s, 1H), 4.39 (s, 1H), 4.15 (s, 1H), 3.09 (s, 3H), 2.50-2.46 (m, 1H), 2.46-2.40 (m, 1H), 2.40-2.29 (m, 2H), 1.38 (s, 3H), 1.36 (s, 3H).

Step 2: To a solution of methyltriphenylphosphonium bromide (5.26 g, 14.7 mmol) in anhydrous THF (23 mL) at −78° C. under an argon atmosphere was added n-butyllithium (5.52 mL, 2.5 M in hexanes, 13.8 mmol) dropwise. The mixture was stirred at room temperature for 0.5 h. A solution of (3aR,5aS,8aS)-4-methoxy-2,2-dimethyltetrahydrocyclopenta[2,3]furo[3,4-d][1,3]dioxol-6(5aH)-one (1.05 g, 4.6 mmol) dissolved in anhydrous THF (23 mL) was added dropwise at −78° C. The reaction was stirred at room temperature for 1 h. The reaction was quenched with saturated aqueous ammonium chloride (50 mL) at 0° C. The mixture was extracted with EtOAc (2×30 mL), and the combined organic layers were washed with brine, dried over anhydrous sodium sulfate, filtered, and concentrated under reduced pressure. The residue was purified by column chromatography on silica (0-40% EtOAc/hexanes) to afford (3aR,4S,5aR,8aR)-4-methoxy-2,2-dimethyl-6-methylidenehexahydrocyclopenta[2,3]furo[3,4-d][1,3]dioxole.

Step 3: To an oven-dried flask containing (3aR,4S,5aR,8aR)-4-methoxy-2,2-dimethyl-6-methylidenehexahydrocyclopenta[2,3]furo[3,4-d][1,3]dioxole (533 mg, 2.4 mmol) dissolved in THF (6 mL) at 0° C. under an atmosphere of argon was added 9-BBN (24 mL, 0.5 M in THF, 12 mmol) dropwise. The reaction was warmed to room temperature and stirred overnight. The mixture was cooled to 0° C. and treated with potassium phosphate tribasic (12 mL, 1 M in water, 12 mmol). The mixture was then stirred for 30 min at room temperature. In a separate vial, a mixture of 7-bromo-3-chloroquinolin-2-amine (910 mg, 3.5 mmol), THF (18 mL), and (2-dicyclohexylphosphino-2′,6′-diisopropoxy-1,1′-biphenyl)[2-(2′-amino-1,1′-biphenyl)]palladium(II) methanesulfonate (200 mg, 0.24 mmol), was purged with nitrogen for 5 min. The stirring quinoline mixture was added to the vial containing the boronate. This reaction was heated at 50° C. for 1.5 h. The mixture was cooled to room temperature and partitioned between brine and EtOAc. The aqueous phase was extracted with EtOAc. The combined organic layers were dried over anhydrous sodium sulfate, filtered, and concentrated under reduced pressure. The residue was purified by column chromatography on silica (0-50% EtOAc/hexanes) to afford 3-chloro-6-{[(3aR,5aR,6S,8aR)-4-methoxy-2,2-dimethylhexahydrocyclopenta[2,3]furo[3,4-d][1,3]dioxol-6-yl]methyl}quinolin-2-amine. MS: 405 (M+1).

Step 4: To a vial containing 3-chloro-6-{[(3aR,5aR,6S,8aR)-4-methoxy-2,2-dimethylhexahydrocyclopenta[2,3]furo[3,4-d][1,3]dioxol-6-yl]methyl}quinolin-2-amine (600 mg, 1.48 mmol) dissolved in acetonitrile (6 mL) were added water (4 mL) and HCl (355 μL, 37% in water, 4.33 mmol). The mixture was heated at 80° C. for 2.5 h, and then stirred overnight at room temperature. The mixture was cooled to 0° C., and quenched with saturated aqueous sodium bicarbonate (364 mg, 4.3 mmol). The aqueous layer was extracted with EtOAc, and the combined organic layers were washed with brine, dried over anhydrous sodium sulfate, filtered, and concentrated under reduced pressure to afford (3R,3aS,6S,6aR)-6-[(2-amino-3-chloroquinolin-7-yl)methyl]hexahydro-3aH-cyclopenta[b]furan-2,3,3a-triol. MS: 351 (M+1). ¹H NMR (DMSO-d6) δ: 8.14 (s, 1H), 7.57 (d, J=8.2 Hz, 1H), 7.34 (s, 1H), 7.13 (dd, J=8.2, 1.4 Hz, 1H), 6.65 (s, 2H), 5.96 (d, J=6.6 Hz, 1H), 5.16 (dd, J=6.6, 4.0 Hz, 1H), 4.66 (d, J=7.6 Hz, 1H), 4.46 (s, 1H), 3.92 (d, J=4.6 Hz, 1H), 3.48 (dd, J=7.5, 4.0 Hz, 1H), 2.82 (dd, J=13.4, 8.4 Hz, 1H), 2.66 (dd, J=13.4, 6.7 Hz, 1H), 2.26-2.12 (m, 1H), 1.83-1.73 (m, 1H), 1.62 (dt, J=12.6, 6.6 Hz, 1H), 1.57-1.46 (m, 1H), 1.31 (qd, J=12.1, 7.1 Hz, 1H).

Intermediates 23-25: Intermediates 23-25 in Table 4 were synthesized using the protocol described in intermediate 22 making the appropriate substitution for the 7-bromo-3-chloroquinolin-2-amine in step 3. The substituted starting material was commercially acquired, synthesized as reported above, or synthesized through known routes reported in the literature.

TABLE 4

| Intermediate | Structure | Name | MS |
|---|---|---|---|
| 23 | 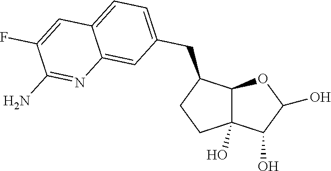 | (3R,3aS,6S,6aR)-6-[(2- amino-3-fluoroquinolin-7- yl)methyl]hexahydro-3aH- cyclopenta[b]furan-2,3,3a- triol | 335 (M + 1) |
| 24 | 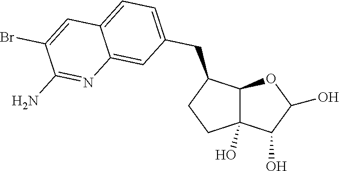 | (3R,3aS,6S,6aR)-6-[(2- amino-3-bromoquinolin-7- yl)methyl]hexahydro-3aH- cyclopenta[b]furan-2,3,3a- triol | 395/397 (M + 1/M + 3) |
| 25 | 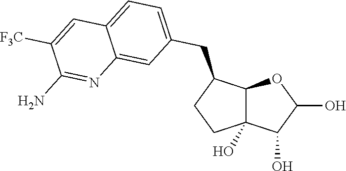 | (3R,3aS,6S,6aR)-6-((2- amino-3- (trifluoromethyl)quinolin-7- yl)methyl)hexahydro-3aH- cyclopenta[b]furan-2,3,3a- triol | 385 (M + 1) |

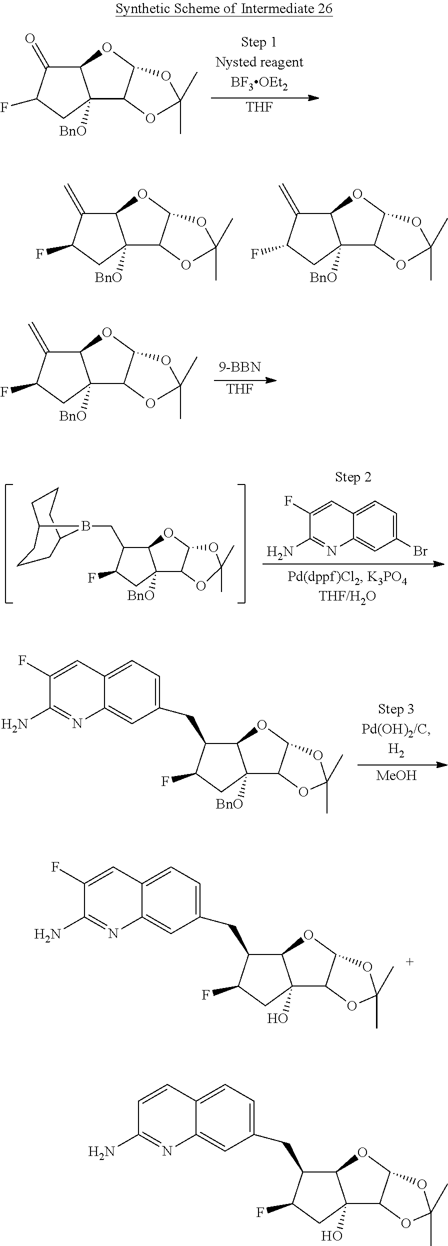

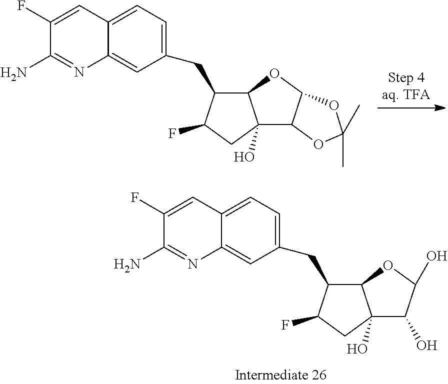

### Intermediate 26: (3R,3aS,5R,6S, 6aR)-6-((2-amino-3-fluoroquinolin-7-yl)methyl)-5-fluorohexahydro-2H-cyclopenta[b]furan-2,3,3a-triol

Step 1: To a mixture of Nysted Reagent (6.37 g, 14.0 mmol) in anhydrous THF (40 mL) was added dropwise boron trifluoride diethyl etherate (1.8 mL, 14.0 mmol) at 0° C. under argon atmosphere. The mixture was stirred at 0° C. for 5 minutes. A solution of (3aR,4aS,7aS,7bR)-7a-(benzyloxy)-6-fluoro-2,2-dimethyltetrahydro-3aH-cyclopenta[4,5]furo[2,3-d][1,3]dioxol-5(4aH)-one (1.5 g, 4.7 mmol) in anhydrous THF (35 mL) was added at 0° C. The resulting mixture was stirred at ambient temperature for 15 h. The reaction mixture was quenched by adding saturated aqueous NaHCO₃ (40 mL) at 0° C., and then it was partitioned between EtOAc/H₂O (250 mL/50 mL). The organic layer was separated, and the aqueous layer was re-extracted with EtOAc (100 mL). The combined organic layers were washed with water (150 mL) and brine (2×100 mL). The organic layer was dried over anhydrous Na₂SO₄, filtered, and the filtrate was concentrated under reduced pressure. The residue was purified by column chromatography on silica (0-10% EtOAc/petroleum ether) to afford (3aR,4aR,6R,7aR,7bR)-7a-(benzyloxy)-6-fluoro-2,2-dimethyl-5-methylenehexahydro-3aH-cyclopenta[4,5]furo[2,3-d][1,3]dioxole. ¹H-NMR (400 MHz, CDCl₃) δ 7.38-7.29 (m, 5H), 5.99 (d, J=3.6 Hz, 1H), 5.68-5.64 (m, 2H), 5.57-5.39 (m, 1H), 4.78 (s, 1H), 4.72 (d, J=10.8 Hz, 1H), 4.64 (d, J=4.0 Hz, 1H), 4.57 (d, J=10.8 Hz, 1H), 2.79-2.72 (m, 1H), 1.96-1.85 (m, 1H), 1.67 (s, 3H), 1.43 (s, 3H). ¹⁹F-NMR (376 MHz, CDCl₃) δ −169.53 (s, 1F). The chromatography step also afforded (3aR,4aR, 6S, 7aR,7bR)-7a-(benzyloxy)-6-fluoro-2,2-dimethyl-5-methylenehexahydro-3aH-cyclopenta[4,5]furo[2,3-d][1,3]dioxole. ¹H-NMR (400 MHz, CDCl₃) δ 7.45-7.44 (m, 2H), 7.44-7.27 (m, 3H), 5.87 (d, J=3.6 Hz, 1H), 5.72 (dd, J=4.4, 1.6 Hz, 2H), 5.58-5.42 (m, 1H), 4.85 (s, 1H), 4.66 (dd, J=17.2, 10.4 Hz, 2H), 4.61 (d, J=4.0 Hz, 1H), 2.67-2.56 (m, 1H), 2.02-1.89 (m, 1H), 1.66 (s, 3H), 1.42 (s, 3H). ¹⁹F-NMR (376 MHz, CDCl₃) δ −164.53 (s, 1F).

Step 2: To a solution of (3aR,4aR,6R,7aR,7bR)-7a-(benzyloxy)-6-fluoro-2,2-dimethyl-5-methylenehexahydro-3aH-cyclopenta[4,5]furo[2,3-d][1,3]dioxole (180 mg, 0.562 mmol) in anhydrous THF (0.5 mL) was added dropwise 9-BBN in THF (0.5 M, 6.7 mL, 3.4 mmol) at 0° C. under argon atmosphere, and the mixture was stirred at 70° C. for 1.5 h. The mixture was cooled to 0° C., and a solution of K₃PO₄ (1M in water, 716 mg, 3.37 mmol) was added. The resultant mixture was stirred for 0.5 h at ambient temperature. Then a solution of 7-bromo-3-fluoroquinolin-2-amine (122 mg, 0.51 mmol) in 3.5 mL of anhydrous THF and Pd(dppf)Cl₂ (41.1 mg, 0.056 mmol) were added to the mixture. The mixture was heated to 80° C. in a microwave reactor for 3.0 h. The organic layer was separated, and the aqueous layer was extracted with EtOAc (150 mL×2). The combined organic layers were washed with water (50 mL) and brine (80 mL). The organic layer was dried over anhydrous Na₂SO₄, filtered, and the filtrate was concentrated under reduced pressure. The residue was purified by column chromatography on silica (0-25% EtOAc/petroleum ether) to afford 7-(((3aR,4aR,5S,6R,7aR,7bR)-7a-(benzyloxy)-6-fluoro-2,2-dimethylhexahydro-3aH-cyclopenta[4,5]furo[2,3-d][1,3]dioxol-5-yl)methyl)-3-fluoroquinolin-2-amine. MS 483 (M+1). ¹H-NMR (400 MHz, Chloroform-d) δ 7.62-7.54 (m, 3H), 7.35-7.33 (m, 4H), 7.31-7.28 (m, 2H), 6.02 (d, J=3.6 Hz, 1H), 5.51 (s, 2H), 5.09-4.93 (m, 1H), 4.72-4.67 (m, 2H), 4.54 (d, J=4.0 Hz, 1H), 4.45 (d, J=10.8 Hz, 1H), 3.14-3.11 (m, 2H), 2.60-2.41 (m, 2H), 2.20-2.07 (m, 1H), 1.62 (s, 3H), 1.44 (s, 3H). ¹⁹F-NMR (376 MHz, Chloroform-d) δ −137.96 (s, 1F), −182.37 (s, 1F).

Step 3: To a mixture of 7-(((3aR,4aR,5S,6R, 7aR,7bR)-7a-(benzyloxy)-6-fluoro-2,2-dimethylhexahydro-3aH-cyclopenta[4,5]furo[2,3-d][1,3]dioxol-5-yl)methyl)-3-fluoroquinolin-2-amine (180 mg, 0.37 mmol) in MeOH (16 mL) and THF (2 mL) was added Pd(OH)₂/C (20 wt. %, 500 mg, 0.71 mmol) at ambient temperature under argon atmosphere. The suspension was degassed under vacuum and purged with H₂ several times, and then it was stirred under 1 atm of H₂ at ambient temperature for 6 h. The mixture was filtered, and the filter cake was washed with MeOH/concentrated aqueous ammonia (10:1) three times (each 10 mL). The filtrate was concentrated under reduced pressure. The residue was purified by column chromatography on silica (0-10% MeOH/DCM) to afford (3aR,4aR,5S,6R, 7aR,7bR)-5-((2-amino-3-fluoroquinolin-7-yl)methyl)-6-fluoro-2,2-dimethylhexahydro-3aH-cyclopenta[4,5]furo[2,3-d][1,3]dioxol-7a-ol. MS: 393 (M+1).

Step 4: (3aR,4aR,5S,6R, 7aR,7bR)-5-((2-amino-3-fluoroquinolin-7-yl)methyl)-6-fluoro-2,2-dimethylhexahydro-3aH-cyclopenta[4,5]furo[2,3-d][1,3]dioxol-7a-ol (40 mg, 0.10 mmol) was dissolved in TFA and H₂O (2.0 mL, TFA/H₂O=1:1) at 0° C. and the mixture was stirred at ambient temperature for 1.0 h. The mixture was co-evaporated with toluene (3×15.0 mL) to dryness. The obtained residue was purified by reverse-phase column chromatograph on C18 (0-95% 5 mM aq. NH₄HCO₃/ACN) to afford (3R,3aS,5R,6S, 6aR)-6-((2-amino-3-fluoroquinolin-7-yl)methyl)-5-fluorohexahydro-2H-cyclopenta[b]furan-2,3,3a-triol. MS: 353 (M+1). ¹H-NMR (300 MHz, CD₃OD) δ 7.70 (d, J=11.4 Hz, 1H), 7.60-7.52 (m, 2H), 7.29 (d, J=8.1 Hz, 1H), 5.37 (d, J=4.2 Hz, 1H), 5.08-4.95 (m, 1H), 4.31-3.71 (m, 2H), 3.10-2.98 (m, 2H), 2.45-2.29 (m, 2H), 2.08-1.93 (m, 1H). ¹⁹F-NMR (282 MHz, CD₃OD) δ −139.28 to −139.34 (m, 1F), −186.24 to −189.90 (m, 1F).

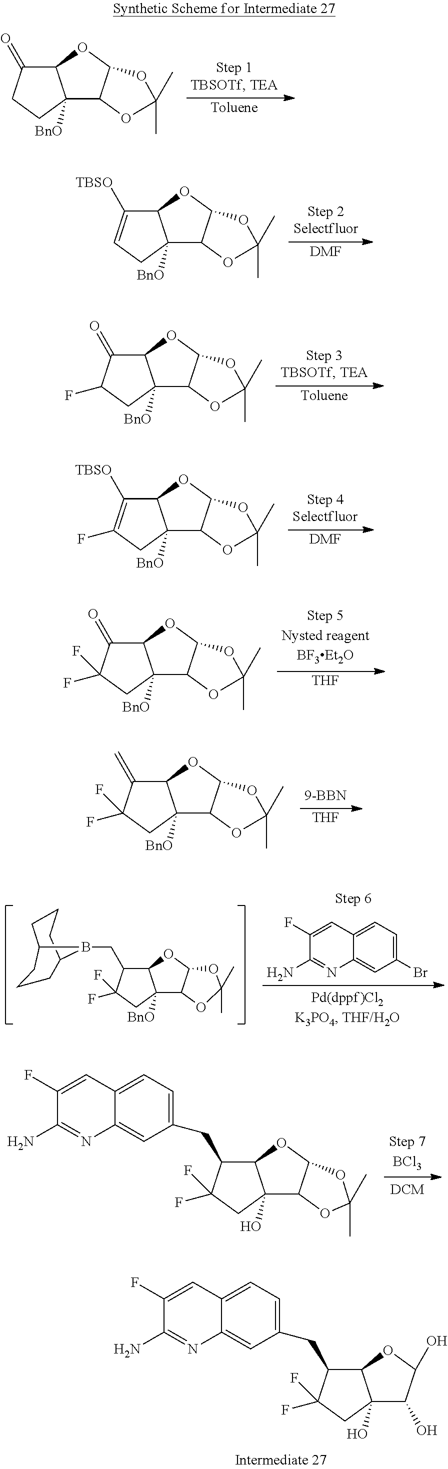

### Intermediate 27: (3R,3aS, 6S, 6aR)-6-((2-amino-3-fluoroquinolin-7-yl)methyl)-5,5-difluorohexahydro-2H-cyclopenta[b]furan-2,3,3a-triol

Step 1: To a mixture of (3aR,4aS,7aS,7bR)-7a-(benzyloxy)-2,2-dimethylhexahydro-5H-cyclopenta[4,5]furo[2,3-d][1,3]dioxol-5-one (3.0 g, 9.9 mmol) in toluene (40 mL) was added triethylamine (46.6 mL, 340 mmol) at ambient temperature under argon atmosphere. The reaction mixture was heated to 100° C. then treated with tert-butyldimethylsilyl trifluoromethanesulfonate (5.21 g, 20. mmol). The resulting mixture was stirred at 100° C. for 30 min. After completion of the reaction, the mixture was cooled to room temperature, diluted with toluene (300 mL), and washed with saturated aqueous NaHCO₃ (300 mL). The organic layer was dried over anhydrous Na₂SO₄ and filtered. The filtrate was concentrated under reduced pressure. The residue was purified by column chromatography on silica (0-15% EtOAc/petroleum ether) to afford (((3aR,4aS,7aR,7bR)-7a-(benzyloxy)-2,2-dimethyl-4a,7,7a,7b-tetrahydro-3aH-cyclopenta[4,5]furo[2,3-d][1,3]dioxol-5-yl)oxy)(tert-butyl)dimethylsilane. ¹H-NMR (300 MHz, DMSO-d₆) δ 7.36-7.26 (m, 5H), 5.85 (d, J=3.3 Hz, 1H), 4.81-4.79 (m, 1H), 4.64-4.56 (m, 3H), 4.46 (s, 1H), 2.70-2.64 (m, 1H), 2.37-2.31 (m, 1H), 1.51 (s, 3H), 1.35 (s, 3H), 0.91 (s, 9H), 0.18 (s, 6H).

Step 2: To a mixture of (((3aR,4aS,7aR,7bR)-7a-(benzyloxy)-2,2-dimethyl-4a,7,7a,7b-tetrahydro-3aH-cyclopenta[4,5]furo[2,3-d][1,3]dioxol-5-yl)oxy)(tert-butyl)dimethylsilane (4.0 g, 9.6 mmol) in anhydrous DMF (70 mL) was added 1-(chloromethyl)-4-fluoro-1,4-diazabicyclo[2.2.2]octane-1,4-diium tetrafluoroborate (3.72 g, 10.5 mmol) at ambient temperature under argon atmosphere. The resulting mixture was stirred at ambient temperature for 2 h. The reaction mixture was diluted with toluene (200 mL) and washed with water (3×50 mL). The combined organic layer was dried over anhydrous Na₂SO₄ and filtered. The filtrate was concentrated under reduced pressure. The residue was purified by column chromatography on silica (0-60% EtOAc/petroleum ether) to afford (3aR,4aS,7aS,7bR)-7a-(benzyloxy)-6-fluoro-2,2-dimethyltetrahydro-3aH-cyclopenta[4,5]furo[2,3-d][1,3]dioxol-5(4aH)-one. ¹H NMR (400 MHz, DMSO-d₆) δ 7.20-7.05 (m, 5H), 5.88-5.67 (m, 1H), 4.62-4.49 (m, 1H), 4.44-4.40 (m, 3H), 3.85-3.83 (m, 1H), 2.13-1.94 (m, 2H), 1.28-1.26 (m, 3H), 1.16-1.14 (m, 3H).

Step 3: To a mixture of (3aR,4aS,7aS,7bR)-7a-(benzyloxy)-6-fluoro-2,2-dimethyltetrahydro-3aH-cyclopenta[4,5]furo[2,3-d][1,3]dioxol-5(4aH)-one (2.0 g, 6.2 mmol) in toluene (10 mL) was added triethylamine (21.4 g, 210 mmol) at ambient temperature under argon atmosphere. The reaction mixture was heated to 100° C. then treated with tert-butyldimethylsilyl trifluoromethanesulfonate (3.28 g, 12.4 mmol). The resulting mixture was stirred at 100° C. for 30 min. After completion of the reaction, the mixture was cooled to room temperature, diluted with water (150 mL), and extracted with EtOAc (3×150 mL). The combined organic layers were dried over anhydrous Na₂SO₄ and filtered. The filtrate was concentrated under reduced pressure. The residue was purified by column chromatography on silica (0-10% EtOAc/petroleum ether) to afford (((3aR,4aS,7aR,7bR)-7a-(benzyloxy)-6-fluoro-2,2-dimethyl-4a,7,7a,7b-tetrahydro-3aH-cyclopenta[4,5]furo[2,3-d][1,3]dioxol-5-yl)oxy)(tert-butyl)dimethylsilane. ¹H-NMR (400 MHz, DMSO-d₆) δ 7.17-7.07 (m, 5H), 5.70-5.56 (m, 1H), 4.52-4.42 (m, 3H), 4.39-4.32 (m, 1H), 2.68 (d, J=16.0 Hz, 1H), 2.51 (d, J=16.4 Hz, 1H), 1.30-1.29 (m, 3H), 1.16-1.15 (m, 3H), 0.72 (s, 9H), −0.04 (s, 6H).

Step 4: To a mixture of (((3aR,4aS,7aR,7bR)-7a-(benzyloxy)-6-fluoro-2,2-dimethyl-4a,7,7a,7b-tetrahydro-3aH-cyclopenta[4,5]furo[2,3-d][1,3]dioxol-5-yl)oxy)(tert-butyl)dimethylsilane (2.6 g, 6.0 mmol) in anhydrous DMF (60 mL) was added 1-(chloromethyl)-4-fluoro-1,4-diazabicyclo[2.2.2]octane-1,4-diium tetrafluoroborate (2.53 g, 7.2 mmol) at 25° C. under argon atmosphere. The resulting mixture was stirred at 25° C. for 2 h. The reaction mixture was diluted with water (300 mL) and extracted with EtOAc (3×300 mL). The combined organic layers were dried over anhydrous Na₂SO₄ and filtered. The filtrate was concentrated under reduced pressure. The residue was purified by column chromatography on silica (0-50% EtOAc/petroleum ether) to afford (3aR,4aS,7aS,7bR)-7a-(benzyloxy)-6,6-difluoro-2,2-dimethyltetrahydro-3aH-cyclopenta[4,5]furo[2,3-d][1,3]dioxol-5(4aH)-one. ¹H-NMR (400 MHz, Chloroform-d) δ 7.42-7.34 (m, 5H), 5.96 (d, J=3.6 Hz, 1H), 4.78 (d, J=10.4 Hz, 1H), 4.62 (d, J=3.6 Hz, 1H), 4.54 (d, J=10.8 Hz, 1H), 4.38 (d, J=4.8 Hz, 1H), 2.90-2.77 (m, 1H), 2.47-2.38 (m, 1H), 1.61 (s, 3H), 1.46 (s, 3H).

Step 5: To a stirred solution of Nysted Reagent (36.9 g, 16.2 mmol, 20 wt. % in THF) in THF (22 mL) was added boron trifluoride diethyl etherate (2.29 g, 16.2 mmol) at 0° C. under argon atmosphere. The mixture was stirred at 0° C. for 5 minutes. A solution of (3aR,4aS,7aS,7bR)-7a-(benzyloxy)-6,6-difluoro-2,2-dimethyltetrahydro-3aH-cyclopenta[4,5]furo[2,3-d][1,3]dioxol-5(4aH)-one (1.1 g, 3.2 mmol) in anhydrous THF (33 mL) was added dropwise into the mixture at 0° C. The resulting mixture was stirred at ambient temperature for 4 h. The reaction mixture was quenched with saturated aqueous NaHCO₃ (50 mL) at 0° C. The resulting mixture was extracted with EtOAc (2×100 mL). The combined organic layers were washed with brine (50 mL), dried over anhydrous Na₂SO₄, filtered, and the filtrate was concentrated under reduced pressure. The residue was purified by Prep-TLC (EtOAc:petroleum ether=1:3) to give (3aR,4aR,7aR,7bR)-7a-(benzyloxy)-6,6-difluoro-2,2-dimethyl-5-methylenehexahydro-3aH-cyclopenta[4,5]furo[2,3-d][1,3]dioxole. ¹H-NMR (400 MHz, DMSO-d₆) δ 7.37-7.26 (m, 5H), 5.97 (d, J=3.6 Hz, 1H), 5.87-5.84 (m, 2H), 4.77 (d, J=4.0 Hz, 1H), 4.70-4.69 (m, 1H), 4.63 (d, J=11.2 Hz, 1H), 4.53 (d, J=11.2 Hz, 1H), 2.98 (t, J=16.0 Hz, 1H), 2.35-2.23 (m, 1H), 1.53 (s, 3H), 1.34 (s, 3H).

Step 6: (3aR,4aR,7aR,7bR)-7a-(benzyloxy)-6,6-difluoro-2,2-dimethyl-5-methylenehexahydro-3aH-cyclopenta[4,5]furo[2,3-d][1,3]dioxole (170 mg, 0.50 mmol) was dissolved in 9-BBN (6.029 mL, 3.01 mmol, 0.5 M in THF) at ambient temperature under argon atmosphere. The resulting solution was stirred at 50° C. for 1 h. The mixture was cooled to 0° C. and treated with a solution of K₃PO₄ (533 mg, 2.50 mmol) in 3.5 mL water. The mixture was stirred for 0.5 h at ambient temperature, then a solution of 7-bromo-3-fluoroquinolin-2-amine (97 mg, 0.40 mmol) in 5.0 mL anhydrous THF and Pd(dppf)Cl₂ (37 mg, 0.05 mmol) were added to the mixture. The mixture was heated to 80° C. in a microwave reactor for 3 h. The mixture was cooled to room temperature, diluted with water (40 mL) and extracted with EtOAc (3×80 mL). The combined organic layers were dried over anhydrous Na₂SO₄, filtered, and the filtrate was concentrated under reduced pressure. The residue was purified by Prep-TLC (EtOAc:petroleum ether=1:2) to give 7-(((3aR,4aR,5S,7aR,7bR)-7a-(benzyloxy)-6,6-difluoro-2,2-dimethylhexahydro-3aH-cyclopenta[4,5]furo[2,3-d][1,3]dioxol-5-yl)methyl)-3-fluoroquinolin-2-amine. MS: 501 (M+1). ¹H-NMR (400 MHz, Chloroform-d) δ 7.61 (s, 1H), 7.55 (d, J=3.6 Hz, 1H), 7.52 (s, 1H), 7.36-7.25 (m, 6H), 5.99 (d, J=3.6 Hz, 1H), 5.61 (s, 2H), 4.68 (d, J=10.4 Hz, 1H), 4.62 (d, J=3.6 Hz, 1H), 4.48 (d, J=10.8 Hz, 1H), 4.39 (dd, J=6.4 Hz, 3.2 Hz, 1H), 3.16-3.05 (m, 2H), 2.87-2.74 (m, 2H), 2.32-2.21 (m, 1H), 1.56 (s, 3H), 1.42 (s, 3H).

Step 7: To a solution of 7-(((3aR,4aR,5S,7aR,7bR)-7a-(benzyloxy)-6,6-difluoro-2,2-dimethylhexahydro-3aH-cyclopenta[4,5]furo[2,3-d][1,3]dioxol-5-yl)methyl)-3-fluoroquinolin-2-amine (290 mg, 0.58 mmol) in anhydrous DCM (6.0 mL) was added dropwise BCl₃ (1 M in DCM, 1.7 mL, 1.74 mmol) at −78° C. under argon atmosphere. The resulting mixture was stirred at −78° C. for 2 h. The reaction mixture was quenched by the addition of triethylamine (0.32 mL, 2.3 mmol), and the resulting mixture was kept at −78° C. for 0.5 h. Then the reaction mixture was poured into saturated aqueous NaHCO₃ (30 mL) at 0° C., and the resulting mixture was stirred at 0° C. for another 0.5 h. The final mixture was extracted with EtOAc (3×200 mL). The combined organic layers were dried over anhydrous Na₂SO₄ and filtered. The filtrate was concentrated under reduced pressure. The residue was purified by reverse-phase column chromatography on C18 (0-95% 5 mM aq. NH₄HCO₃/MeCN) to afford (3R,3aS,6S,6aR)-6-((2-amino-3-fluoroquinolin-7-yl)methyl)-5,5-difluorohexahydro-2H-cyclopenta[b]furan-2,3,3a-triol. MS: 371 (M+1). ¹H-NMR (300 MHz, DMSO-d₆) δ 7.81 (d, J=12.0 Hz, 1H), 7.61 (d, J=8.1 Hz, 1H), 7.41 (s, 1H), 7.19 (d, J=8.1 Hz, 1H), 6.81-6.75 (m, 2H), 6.21 (d, J=7.2 Hz, 1H), 5.26-5.22 (m, 1H), 5.01-4.95 (m, 2H), 4.00-3.99 (m, 1H), 3.66-3.62 (m, 1H), 2.97-2.66 (m, 3H), 2.42-2.09 (m, 2H).

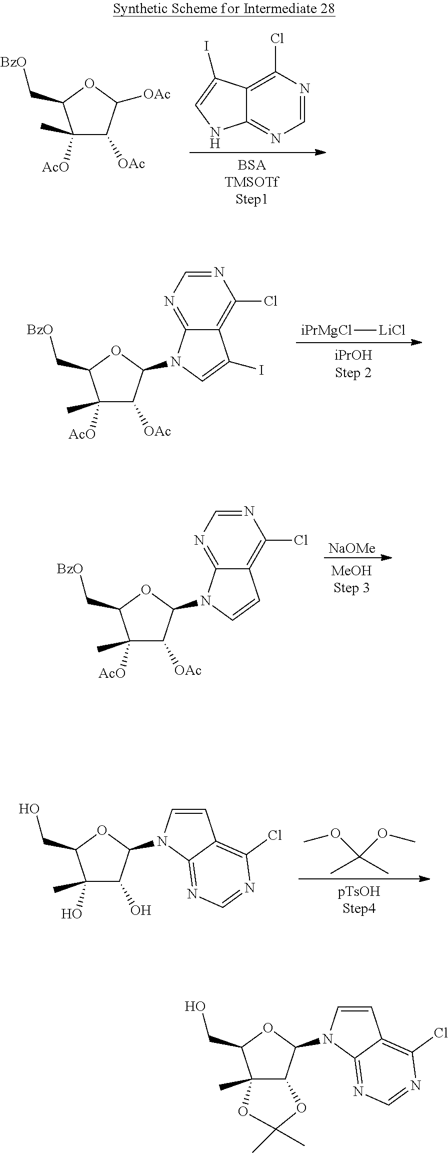

### Intermediate 28: ((3aR,4R,6R,6aR)-6-(4-chloro-7H-pyrrolo[2,3-d]pyrimidin-7-yl)-2,2,3a-trimethyltetrahydrofuro[3,4-d][1,3]dioxol-4-Yl)methanol

Step 1: A solution of 4-chloro-5iodo-7H-pyrolo[2,3-d]pyrimidine (1.417 g, 5.07 mmol) in dry ACN (10 mL) was stirred with BSA (1.25 mL, 5.07 mmol) at room temperature for 15 minutes. (3R,4R,5R)-5-((benzoyloxy)methyl)-4-methyltetrahydrofuran-2,3,4-triyl triacetate (2 g, 5.07 mmol) in ACN (20 mL) was added followed by TMSOTf (1.84 mL, 10.1 mmol), and the reaction mixture was stirred for a further 10 minutes at room temperature, followed by 3 h at 80° C. The reaction mixture was cooled to room temperature and diluted with EtOAc (40 mL). The reaction mixture was then washed with saturated aqueous NaHCO₃ (2×30 mL) and brine (2×30 mL), and dried. The residue was purified by column chromatography on silica gel (PE/Et₂O) to afford (2R,3R,4R,5R)-2-((benzoyloxy)methyl)-5-(4-chloro-5-iodo-7H-pyrrolo[2,3-d]pyrimidin-7-yl)-3-methyltetrahydrofuran-3,4-diyl diacetate. MS: 614 (M+1)

Step 2: To a stirred solution of (2R,3R,4R,5R)-2-((benzoyloxy)methyl)-5-(4-chloro-3-iodo-1H-indol-1-yl)-3-methyltetrahydrofuran-3,4-diyl diacetate (4.6 g, 7.5 mmol) in dry THF (45 mL) was dropwise added isopropylmagnesium chloride-lithium chloride complex (7.21 mL, 9.37 mmol) over a period of 5 minutes at −78° C. The mixture was stirred at −78° C. for 20 minutes, and then quenched with dropwise addition of i-PrOH (0.808 mL, 10.5 mmol) at −78° C. The reaction mixture was poured into a mixture of ice and saturated aqueous NH₄Cl, and extracted with DCM. The organic layers were combined, dried, and concentrated under reduced pressure to afford (2R,3R,4R,5R)-2-((benzoyloxy)methyl)-5-(4-chloro-7H-pyrrolo[2,3-d]pyrimidin-7-yl)-3-methyltetrahydrofuran-3,4-diyl diacetate as an oil. MS: 489 (M+1)

Step 3: At 0° C., sodium methoxide (7.75 mL, 3.87 mmol) was added to a stirred solution of (2R,3R,4R,5R)-2-((benzoyloxy)methyl)-5-(4-chloro-1H-indol-1-yl)-3-methyltetrahydrofuran-3,4-diyl diacetate (3.15 g, 6.46 mmol) in MeOH (100 mL). The mixture was stirred at 0° C. for 1 h and then at room temperature for 2 h. The reaction mixture was quenched with Dowex until pH=6. The reaction mixture was concentrated under reduced pressure, and the residue was purified by column chromatography on silica gel (0-10% MeOH/DCM) to afford (2R,3S,4R,5R)-5-(4-chloro-7H-pyrrolo[2,3-d]pyrimidin-7-yl)-2-(hydroxymethyl)-3-methyltetrahydrofuran-3,4-diol as a foam. MS: 300 (M+1)

Step 4: A mixture of (2R,3S,4R,5R)-5-(4-chloro-1H-indol-1-yl)-2-(hydroxymethyl)-3-methyltetrahydrofuran-3,4-diol (443 mg, 1.48 mmol), p-toluenesulfonic acid monohydrate (562 mg, 2.96 mmol) and 2,2-dimethoxypropane (1.844 μl, 14.78 mmol) in acetone (35 mL) was stirred at 65° C. overnight. The reaction mixture was extracted with DCM and the organic phase was washed with saturated aqueous NaHCO₃. The organic phase was dried, concentrated under reduced pressure, and the residue was purified by reverse phase HPLC (C18, ACN/water) to afford ((3aR,4R,6R,6aR)-6-(4-chloro-7H-pyrrolo[2,3-d]pyrimidin-7-yl)-2,2,3a-trimethyltetrahydrofuro[3,4-d][1,3]dioxol-4-yl)methanol as a foam. MS: 363 (M+24)

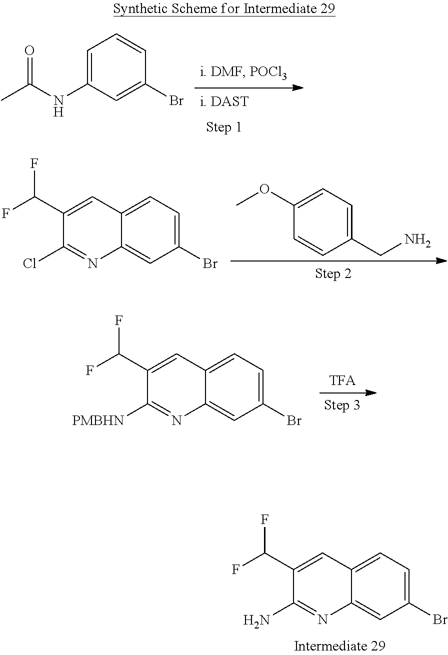

### Intermediate 29: 7-bromo-3-(difluoromethyl)quinolin-2-amine

Step 1: To DMF (16 mL) was added POCl₃ (48.8 mL, 523 mmol) dropwise via cannula over 30 minutes at 0° C., and the reaction mixture was stirred for another 30 minutes at this temperature. Then N-(3-bromophenyl)acetamide (16 g, 75 mmol) was added to the mixture and the reaction was stirred at 80° C. for 2 h. The solvent was then removed under reduced pressure to afford crude residue which was diluted with 200 mL of saturated aqueous NaHCO₃ and extracted with 1000 mL of EtOAc. The organic phase was washed with water (600 mL), brine (300 mL), dried over anhydrous sodium sulfate, filtered, and the filtrate was concentrated under reduced pressure. The resulting residue was purified by silica gel column chromatography (eluted with 20% EtOAc/PE) to afford 7-bromo-2-chloroquinoline-3-carbaldehyde as a solid. Then 7-bromo-2-chloroquinoline-3-carbaldehyde (1.8 g, 6.65 mmol) was co-evaporated with toluene (5 mL) three times. To a solution of 7-bromo-2-chloroquinoline-3-carbaldehyde (1.8 g, 6.65 mmol) in DCM (27 mL) was added DAST (1.76 mL, 13.31 mmol) at 0° C., and the mixture was then stirred at 50° C. for 1.5 h. The reaction was diluted with 50 mL of saturated aqueous NaHCO₃ at 0° C. and extracted with 250 mL EtOAc. The organic phase was washed with water (100 mL), brine (100 mL), dried over anhydrous sodium sulfate, filtered, and the filtrate was concentrated under reduced pressure. The resulting residue was purified by silica gel column chromatography (eluted with 30% DCM/PE) to afford 7-bromo-2-chloro-3-(difluoromethyl)quinoline as a solid. MS: 292/294 (M+1/M+3).

Step 2: A solution of 7-bromo-2-chloro-3-(difluoromethyl)quinoline (960 mg, 3.28 mmol) and (4-methoxyphenyl)methanamine (2.144 mL, 16.41 mmol) in 1,4-dioxane (10 mL) was stirred at room temperature in a sealed tube. Then the reaction mixture was heated at 90° C. for 16 h. The reaction was concentrated under reduced pressure, and the resulting residue was purified by silica gel column chromatography (eluted with 20% EtOAc/PE) to afford 7-bromo-3-(difluoromethyl)-N-(4-methoxybenzyl)quinolin-2-amine as a solid. MS: 393/395 (M+1/M+3).

Step 3: A solution of 7-bromo-3-(difluoromethyl)-N-(4-methoxybenzyl)quinolin-2-amine (200 mg, 0.509 mmol) in TFA (15 mL) was stirred at 50° C. under argon for 3 h. The reaction was diluted with 100 mL of saturated aqueous NaHCO₃ at 0° C. and extracted with 200 mL of EtOAc. The organic phase was washed with water (50 mL), brine (50 mL), dried over anhydrous sodium sulfate, filtered, and the filtrate was concentrated under reduced pressure. The resulting residue was purified by silica gel column chromatography (eluted with 20% EtOAc/PE) to afford 7-bromo-3-(difluoromethyl)quinolin-2-amine as a solid. MS: 273/275 (M+1/M+3).

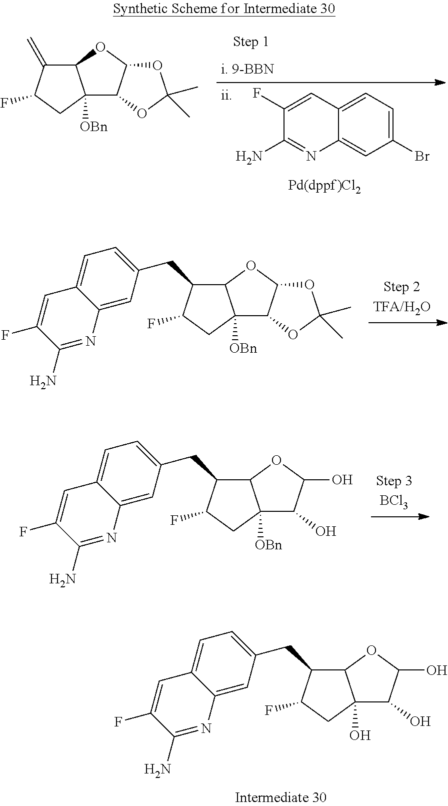 [SMILES: C=C1[C@H]2O[C@@H]3OC(C)(C)O[C@@H]3[C@@]2(OCc2ccccc2)C[C@@H]1F ; Nc1nc2cc(Br)ccc2cc1F ; CC1(C)O[C@H]2OC3[C@H](Cc4ccc5cc(F)c(N)nc5c4)[C@@H](F)C[C@]3(OCc3ccccc3)[C@H]2O1 ; Nc1nc2cc(C[C@H]3C4OC(O)[C@H](O)[C@@]4(OCc4ccccc4)C[C@@H]3F)ccc2cc1F ; Nc1nc2cc(C[C@H]3C4OC(O)[C@H](O)[C@@]4(O)C[C@@H]3F)ccc2cc1F]

### Intermediate 30: (3R,3aS,5S,6S)-6-((2-amino-3-fluoroquinolin-7-yl)methyl)-5-fluorohexahydro-2H-cyclopenta[b]furan-2,33a-triol

Step 1: To a solution of (3aR,4aR,6S,7aR,7bR)-7a-(benzyloxy)-6-fluoro-2,2-dimethyl-5-methylenehexahydro-3aH-cyclopenta[4,5]furo[2,3-d][1,3]dioxole (190 mg, 0.593 mmol) in anhydrous THF (0.5 mL) was added 9-BBN (7.12 mL, 0.5M in THF, 3.56 mmol) dropwise at room temperature under argon. The mixture was stirred at 70° C. for 1.5 h. Then the mixture was cooled to 0° C., and a solution of K₃PO₄ (755 mg, 3.56 mmol) in 2.5 mL of H₂O was added. The resultant mixture was stirred for another 0.5 h at room temperature. Then a solution of 7-bromo-3-fluoroquinolin-2-amine (129 mg, 0.534 mmol) in anhydrous THF (3 mL) and Pd(dppf)Cl2 (43.4 mg, 0.059 mmol) were added to the mixture. The final reaction mixture was irradiated with microwave radiation at 80° C. for 3 h. The organic layer was then separated, and the aqueous layer was re-extracted with EtOAc (60 mL×2). The combined organic layers were washed with H₂O (60 mL) and brine (60 mL), dried over anhydrous Na₂SO₄, filtered, and the filtrate was concentrated under reduced pressure. The resulting residue was purified by silica gel flash chromatography (eluted with 0-25% EtOAc/PE) to afford 7-(((3aR,4aR,5S,6S,7aR,7bR)-7a-(benzyloxy)-6-fluoro-2,2-dimethylhexahydro-3aH-cyclopenta[4,5]furo[2,3-d][1,3]dioxol-5-yl)methyl)-3-fluoroquinolin-2-amine as a solid. MS: 483 (M+1). ¹H-NMR (300 MHz, DMSO-d₆) δ 7.81 (d, J=11.7 Hz, 1H), 7.61 (d, J=8.1 Hz, 1H), 7.47-7.36 (m, 5H), 7.34-7.29 (m, 1H), 7.15-7.12 (m, 1H), 6.76 (br s, 2H), 5.86 (d, J=3.9 Hz, 1H), 5.42-5.34 (m, 1H), 4.68-4.65 (m, 2H), 4.57 (d, J=11.1 Hz, 1H), 4.22 (s, 1H), 3.02 (dd, J=14.1, 6.9 Hz, 1H), 2.86-2.78 (m, 1H), 2.70-2.64 (m, 2H), 2.08-1.90 (m, 1H), 1.40 (s, 3H), 1.30 (s, 3H).

Step 2: 7-(((3aR,4aR,5S,6S,7aR,7bR)-7a-(benzyloxy)-6-fluoro-2,2-dimethylhexahydro-3aH-cyclopenta[4,5]furo[2,3-d][1,3]dioxol-5-yl)methyl)-3-fluoroquinolin-2- amine (750 mg, 1.55 mmol) was dissolved in TFA and H₂O (12.0 mL, 1:1 TFA/H₂O) at 0° C. and the mixture was then stirred at room temperature for 1 h. The mixture was co-evaporated with toluene (3×20 mL) under reduced pressure. The residue was purified by silica gel column chromatography (eluted with 1-10% MeOH/DCM) to afford (3R,3aS,5S,6S)-6-((2-amino-3-fluoroquinolin-7-yl)methyl)-3a-(benzyloxy)-5-fluorohexahydro-2H-cyclopenta[b]furan-2,3-diol as a solid. MS: 443 (M+1).

Step 3: To a solution of (3R,3aS,5S,6S)-6-((2-amino-3-fluoroquinolin-7-yl)methyl)-3a-(benzyloxy)-5-fluorohexahydro-2H-cyclopenta[b]furan-2,3-diol (650 mg, 1.47 mmol) in anhydrous DCM (20 mL) was added BCl₃ (4.41 mL, 1M in DCM, 4.41 mmol) dropwise at −78° C. under argon. The resulting solution was stirred at −78° C. for 1 h. The reaction was quenched with triethylamine (0.819 mL, 5.88 mmol) and stirred at −78° C. for 0.5 h. The reaction mixture was poured into ice-cold saturated aqueous NaHCO₃ (50 mL) at 0° C. and stirring continued for 0.5 h. The mixture was then extracted with EtOAc (3×200 mL). The combined organic layers were dried over anhydrous Na₂SO₄ and filtered. The filtrate was concentrated under reduced pressure, and the resulting residue and all of the aqueous phase were purified by RP-Combi-Flash at room temperature (ACN/water with 5 mM NH₄CO₃ modifier) to afford (3R,3aS,5S,6S)-6-((2-amino-3-fluoroquinolin-7-yl)methyl)-5-fluorohexahydro-2H-cyclopenta[b]furan-2,3,3a-triol as a solid. MS: 353 (M+1). ¹H NMR (300 MHz, CD₃OD) δ 7.71 (d, J=11.4 Hz, 1H), 7.61 (d, J=8.1 Hz, 1H), 7.50 (s, 1H), 7.25 (d, J=8.1 Hz, 1H), 5.29-5.21 (m, 1H), 5.03-4.97 (m, 1H), 4.39-4.20 (m, 1H), 3.82-3.67 (m, 1H), 3.06-2.99 (m, 2H), 2.68-2.40 (m, 1H), 2.33-2.05 (m, 2H).

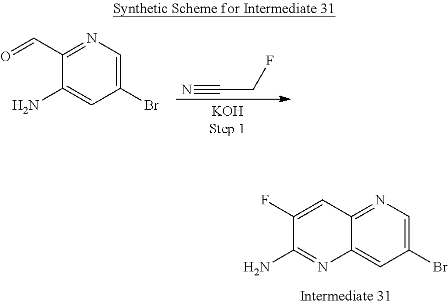

### Intermediate 31: 7-bromo-3-fluoro-1,5-naphthyridin-2-amine

Step 1: 3-amino-5-bromopicolinaldehyde (1000 mg, 4.97 mmol) was dissolved in DMSO (10 mL), charged with 2-fluoroacetonitrile (1108 μL, 19.9 mmol), 15M potassium hydroxide (100 μL, 1.49 mmol) and heated to 80° C. for 2 h. The reaction was poured into 10 mL water, diluted with EtOAc (30 mL) and filtered through Celite. The organic layer was washed with brine, dried over sodium sulfate, filtered, and concentrated under reduced pressure. The residue was purified by column chromatography on silica (0-100% EtOAc/CH₂Cl₂) to afford 7-bromo-3-fluoro-1,5-naphthyridin-2-amine as a solid. MS: 242/244 (M+1/3).

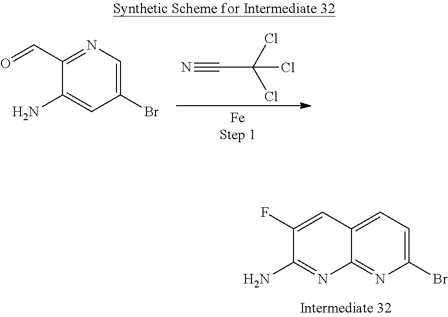 [SMILES: Nc1cc(Br)cnc1C=O ; N#CC(Cl)(Cl)Cl ; Nc1nc2nc(Br)ccc2cc1F]

### Intermediate 32: 7-bromo-3-chloro-1,8-naphthyridin-2-amine

Step 1: A mixture of 2-amino-6-bromonicotinaldehyde (2.6 g, 12.9 mmol), and iron powder (7.22 g, 129 mmol) was degassed under nitrogen, and then charged with THF (26 mL). Trichloroacetonitrile (1.95 mL, 19.4 mmol) was added and the mixture was stirred for 2 h at room temperature. The reaction was refluxed at 65° C. overnight. The reaction was cooled to room temperature and filtered through Celite charged with 10 g of silica gel. The mixture was concentrated under reduced pressure, and the residue was purified by column chromatography on silica (0-50% 3:1 EtOAc:EtOH/Hexanes with 1% aqueous NH₄OH modifier). The resulting solid was washed with 2×10 mL cold Et₂O to afford 7-bromo-3-chloro-1,8-naphthyridin-2-amine as a solid used without further purification. MS: 258/260 (M+1/3).

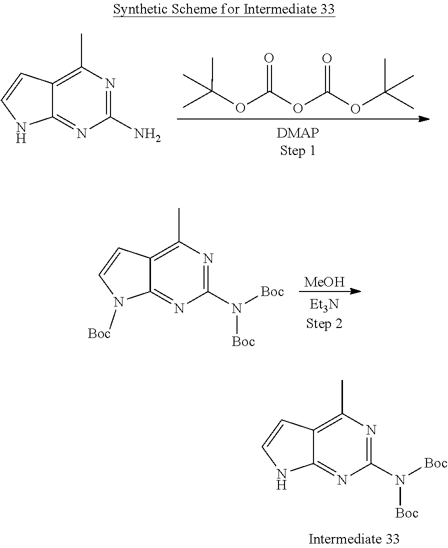 [SMILES: "MeOH" ; Cc1nc(N)nc2[nH]ccc12 ; CC(C)(C)OC(=O)OC(=O)OC(C)(C)C ; Cc1nc(N(C(=O)OC(C)(C)C)C(=O)OC(C)(C)C)nc2c1ccn2C(=O)OC(C)(C)C ; Cc1nc(N(C(=O)OC(C)(C)C)C(=O)OC(C)(C)C)nc2[nH]ccc12]

### Intermediate 33: di-tert-butyl (4-methyl-7H-pyrrolo[2,3-d]pyrimidin-2-yl)imidodicarbonate

Step 1: To a solution of 4-methyl-7H-pyrrolo[2,3-d]pyrimidin-2-amine (0.5 g, 3.4 mmol) in acetonitrile (8.5 mL)/DCM (8.5 mL) was added di-tert-butyl dicarbonate (2.6 g, 12 mmol) and 4-dimethylaminopyridine (0.082 g, 0.68 mmol). The solution was stirred for 18 h at room temperature. The reaction was concentrated and purified by column chromatography on silica (0-60% EtOAc/Hexanes) to afford tert-butyl 2-[bis(tert-butoxycarbonyl)amino]-4-methyl-7H-pyrrolo[2,3-d]pyrimidine-7-carboxylate as a solid. MS: 449 (M+1).

Step 2: To a solution of tert-butyl 2-[bis(tert-butoxycarbonyl)amino]-4-methyl-7H-pyrrolo[2,3-d]pyrimidine-7-carboxylate (0.6 g, 1.34 mmol) in MeOH (2.2 mL) was added triethylamine (1.87 mL, 13.4 mmol) at room temperature. The reaction was heated to 60° C. and stirred for 18 h. The mixture was cooled to room temperature and concentrated under reduced pressure. The residue was purified by column chromatography on silica (0-100% EtOAc/Hexanes) to afford di-tert-butyl (4-methyl-7H-pyrrolo[2,3-d]pyrimidin-2-yl)imidodicarbonate. MS: 349 (M+1).

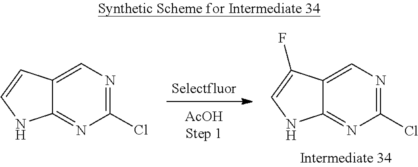 [SMILES: Clc1ncc2cc[nH]c2n1 ; Fc1c[nH]c2nc(Cl)ncc12]

### Intermediate 34: 2-chloro-5-fluoro-1H-pyrrolo[2,3-d]pyrimidine

To 2-chloro-1H-pyrrolo[2,3-d]pyrimidine (335 mg, 2.18 mmol) in acetonitrile (11 mL) was added Selectfluor (1.16 g, 3.27 mmol) and AcOH (1.1 mL). The mixture was heated at 70° C. overnight. The mixture was cooled to room temperature and concentrated under reduced pressure. The residue was diluted with EtOAc and washed with water (2×). The solution was dried over sodium sulfate, filtered, and concentrated under reduced pressure. The residue was purified by chromatography on silica (20-50% EtOAc/Hexanes) to afford 2-chloro-5-fluoro-1H-pyrrolo[2,3-d]pyrimidine. MS: 172 (M+1).

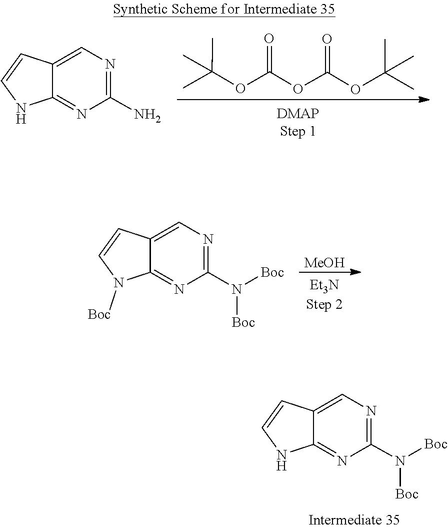 [SMILES: "MeOH" ; Nc1ncc2cc[nH]c2n1 ; CC(C)(C)OC(=O)OC(=O)OC(C)(C)C ; CC(C)(C)OC(=O)N(C(=O)OC(C)(C)C)c1ncc2ccn(C(=O)OC(C)(C)C)c2n1 ; CC(C)(C)OC(=O)N(C(=O)OC(C)(C)C)c1ncc2cc[nH]c2n1]

### Intermediate 35: bis(2-methyl-2-propanyl) 7H-pyrrolo[2,3-d]pyrimidin-2-ylimidodicarbonate

Step 1: To a stirred solution of 7H-pyrrolo[2,3-d]pyrimidin-2-amine (500 mg, 3.73 mmol) in acetonitrile (9 mL) and dichloromethane (9 mL) was added Boc-anhydride (2.85 g, 13.1 mmol) and DMAP (91 mg, 0.75 mmol). The reaction mixture was stirred overnight. The mixture was concentrated under reduced pressure, and the residue was purified by column chromatography on silica (0-40% EtOAc in Hex) to afford 2-methyl-2-propanyl 2-(bis{[(2-methyl-2-propanyl)oxy]carbonyl}amino)-7H-pyrrolo[2,3-d]pyrimidine-7-carboxylate. MS: 435 (M+1).

Step 2: To a stirred solution of 2-methyl-2-propanyl 2-(bis{[(2-methyl-2-propanyl)oxy]carbonyl}amino)-7H-pyrrolo[2,3-d]pyrimidine-7-carboxylate (1.52 g, 3.50 mmol) in MeOH (17.5 mL) was added TEA (4.88 mL, 35.0 mmol). The solution was heated at reflux for 2.5 h. The mixture was cooled to room temperature, concentrated under reduced pressure, and purified by silica gel chromatography (0-60% EtOAc in Hex) to afford bis(2-methyl-2-propanyl) 7H-pyrrolo[2,3-d]pyrimidin-2-ylimidodicarbonate. MS: 335 (M+1). ¹H NMR (500 MHz, DMSO-d₆) δ 12.23 (s, 1H), 8.98 (s, 1H), 7.62 (d, J=3.5 Hz, 1H), 6.63 (d, J=3.5 Hz, 1H), 1.38 (s, 18H).

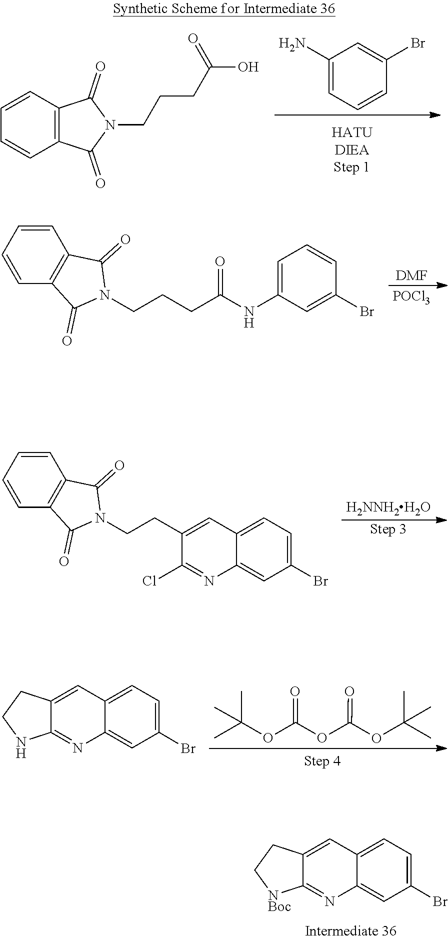

### Intermediate 36: tert-butyl 7-bromo-2,3-dihydro-1H-pyrrolo[2,3-b]quinoline-1-carboxylate

Step 1: To a solution of 4-(1,3-dioxoisoindolin-2-yl)butanoic acid (7.73 g, 33.1 mmol), HATU (15.1 g, 39.8 mmol) and DIEA (17.4 mL, 99 mmol) in DMF (50 mL) was added 3-bromoaniline (5.7 g, 33.1 mmol) at 15° C. The mixture was stirred for 0.5 h. Water (500 mL) was added and the mixture was extracted with EtOAc (200 mL×3). The combined organic layers were washed with brine (200 mL) and concentrated under reduced pressure. The residue was purified by filtering with EtOAc to afford N-(3-bromophenyl)-4-(1,3-dioxoisoindolin-2-yl)butanamide as a solid. MS:387/389 (M+1/M+3)

Step 2: DMF (2.70 mL, 34.9 mmol) was added dropwise to POCl₃ (19.02 mL, 204 mmol) at 5° C. (temperature kept within 5-15° C.), and the reaction mixture was stirred for 15 minutes. N-(3-bromophenyl)-4-(1,3-dioxoisoindolin-2-yl)butanamide (9 g, 23.24 mmol) was added to the reaction mixture and heated to 80° C. for 12 hours. The mixture was cooled to room temperature and poured into water (200 mL), and the pH was adjusted to 9. The mixture was extracted with EtOAc (100 mL×3), and the combined organic layers were concentrated under reduced pressure. The residue was purified by filtering with EtOAc to afford 2-(2-(7-bromo-2-chloroquinolin-3-yl)ethyl)isoindoline-1,3-dione as a solid. MS: 415/417 (M+1/M+3)

Step 3: Hydrazine hydrate (0.905 mL, 18.2 mmol) was added dropwise to 2-(2-(7-bromo-2-chloroquinolin-3-yl)ethyl)isoindoline-1,3-dione (6.3 g, 15.2 mmol) in butan-1-ol (60 mL) at 80° C. The reaction mixture was stirred at 100° C. for 12 h. The reaction was concentrated under reduced pressure to afford 7-bromo-2,3-dihydro-1H-pyrrolo[2,3-b]quinoline as a solid. MS: 249/251 (M+1/M+3)

Step 4: Into a 5 L 4-necked round bottom flask purged and maintained with an inert atmosphere of nitrogen was added 7-bromo-2,3-dihydro-1H-pyrrolo[2,3-b]quinoline (100 g, 0.401 mol) and di-tert-butyl dicarbonate (400 g, 1.83 mol). The resulting solution was stirred for 12 h at 100° C. The mixture was cooled to room temperature and concentrated under reduced pressure. The residue was purified by column chromatography on silica (1:10 ethyl acetate/petroleum ether) to afford tert-butyl 7-bromo-2,3-dihydro-1H-pyrrolo[2,3-b]quinoline-1-carboxylate as a solid. MS: 349/351 (M+1/M+3).

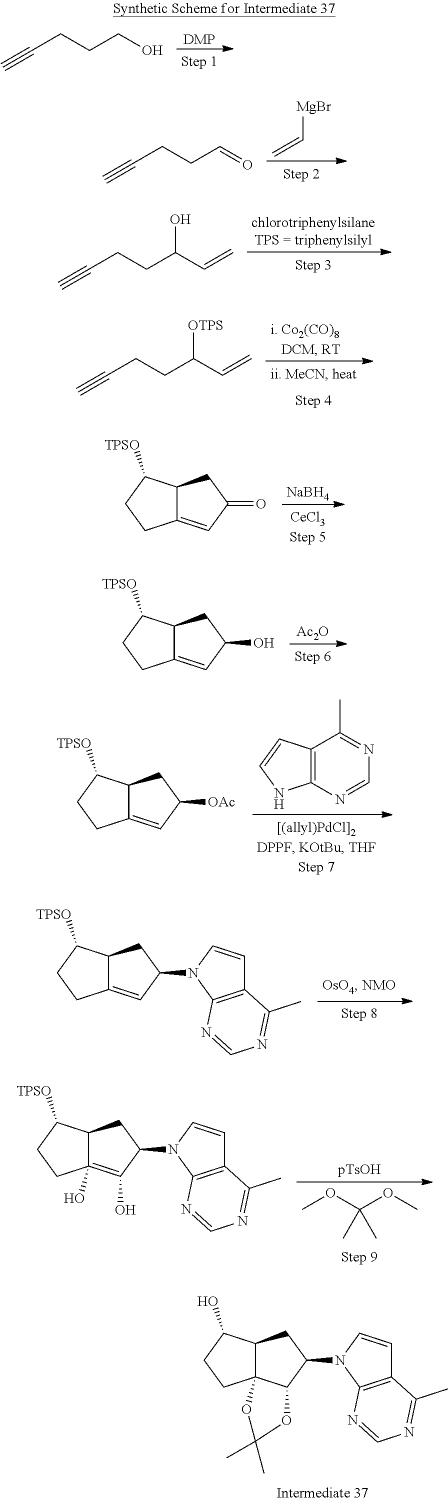

### Intermediate 37: (3aS,4R,5aR,6S,8aR)-2,2-dimethyl-4-(4-methyl-7H-pyrrolo[2,3-d]pyrimidin-7-yl)hexahydro-5H-pentaleno[1,6a-d][1,3]dioxol-6-ol

Step 1: To a flask containing a solution of pent-4-yn-1-ol (2.4 mL, 25 mmol) in DCM (200 mL) was added Dess-Martin Periodinane (14 g, 33 mmol). The reaction was stirred at room temperature overnight. The reaction was slowly poured into a beaker containing a stirring solution of both saturated aqueous sodium bicarbonate and saturated aqueous sodium thiosulfate. The mixture was poured into a separatory funnel and extracted. The organic layers were combined, dried over magnesium sulfate, filtered through a plug of Celite®, and concentrated under reduced pressure to afford pent-4-ynal which was used in the next step without further purification.

Step 2: To a flask containing the crude pent-4-ynal was added THF (200 mL). The reaction was cooled to 0° C. under an atmosphere of argon. Vinyl magnesium bromide (50 mL, 1 M, 50 mmol) was added and the reaction was stirred at 0° C. for 70 minutes. The reaction was poured into a separatory funnel containing saturated aqueous ammonium chloride and extracted with EtOAc. The organic layers were combined, dried over magnesium sulfate, filtered, and concentrated under reduced pressure to afford hept-1-en-6-yn-3-ol which was used in the next step without further purification.

Step 3: To a flask containing the crude hept-1-en-6-yn-3-ol in DCM (200 mL), was added pyridine (6.0 mL, 74 mmol), DMAP (4.58 g, 37.5 mmol), and triphenylchlorosilane (11.5 g, 37.5 mmol). The reaction was stirred at room temperature overnight. The reaction was poured into a separatory funnel containing saturated aqueous ammonium chloride and extracted. The organic layers were combined, dried over magnesium sulfate, filtered, and concentrated under reduced pressure. The residue was then purified by column chromatography on silica (0-10% EtOAc/hexanes) to afford (hept-1-en-6-yn-3-yloxy)triphenylsilane. ¹H NMR (600 MHz, CDCl₃) δ 7.65-7.61 (m, 6H), 7.45-7.41 (m, 3H), 7.39-7.35 (m, 6H), 5.85-5.78 (m, 1H), 5.06-4.99 (m, 2H), 4.42 (q, J=6.2 Hz, 1H), 2.27-2.15 (m, 2H), 1.87-1.78 (m, 2H), 1.77-1.70 (m, 1H).

Step 4: To a flask containing a solution of (hept-1-en-6-yn-3-yloxy)triphenylsilane (4.72 g, 12.8 mmol) in DCM (250 mL) was added dicobalt octacarbonyl (5.25 g, 14.6 mmol), under an atmosphere of argon. The reaction was stirred at room temperature for 2 h. The reaction was concentrated under reduced pressure, and the residue was dissolved in acetonitrile (500 mL). The reaction was heated to 83° C. under an atmosphere of argon for overnight. The reaction was concentrated under reduced pressure, triturated with ether, filtered over a plug of Celite®, and then the filtrate was concentrated under reduced pressure. The residue was purified by column chromatography on silica (10-30% EtOAc/hexanes) followed by chiral SFC (R,R′-Welk-O1 column, 20% MeOH w/ 0.1% NH₄OH in CO₂) to afford (6R,6aR)-6-((triphenylsilyl)oxy)-4,5,6,6a-tetrahydropentalen-2(1H)-one. ¹H NMR (600 MHz, CDCl₃) δ 7.65-7.61 (m, 6H), 7.47-7.43 (m, 3H), 7.42-7.37 (m, 6H), 5.80-5.78 (m, 1H), 3.93 (q, J=8.8 Hz, 1H), 3.18-3.12 (m, 1H), 2.83-2.74 (m, 1H), 2.49-2.41 (m, 1H), 2.37 (dd, J=18.1, 6.2 Hz, 1H), 2.21-2.10 (m, 2H), 1.73 (dd, J=18.1, 3.1 Hz, 1H).

Step 5: To a flask containing (6R,6aR)-6-((triphenylsilyl)oxy)-4,5,6,6a-tetrahydropentalen-2(1H)-one (7.13 g, 18 mmol) was added THF (100 mL) and methanol (80 mL). The solution was cooled in a dry ice/MeCN bath, and then cerium(III) chloride heptahydrate (6.70 g, 18.0 mmol) was added. The reaction was stirred in the bath for 20 minutes before sodium borohydride (0.817 g, 22 mmol) was added. The reaction was stirred in the cold bath for another 20 minutes before being brought out of the bath. After 5 minutes, the reaction was poured into a separatory funnel containing EtOAc and 3:2:1 saturated ammonium chloride:water:brine (200 mL). The aqueous layer was separated and washed twice more with EtOAc. The combined organic layers were then dried over sodium sulfate, filtered over Celite®, and concentrated under reduced pressure. The crude (2R,6S,6aS)-6-((triphenylsilyl)oxy)-1,2,4,5,6,6a-hexahydropentalen-2-ol was taken directly to the next step.

Step 6: To a flask containing the crude (2R,6S,6aS)-6-((triphenylsilyl)oxy)-1,2,4,5,6,6a-hexahydropentalen-2-ol in DCM (120 mL) was added pyridine (2.9 mL, 36 mmol), DMAP (2.86 g, 23.4 mmol), and acetic anhydride (2.2 mL, 23 mmol). The reaction was stirred at room temperature for three days. The reaction was quenched with saturated aqueous ammonium chloride (80 mL). The organic layer was separated by a Phase Separator, concentrated under reduced pressure, and purified by column chromatography on silica (0-10% EtOAc/hexanes) to afford (2R,6S,6aS)-6-((triphenylsilyl)oxy)-1,2,4,5,6,6a-hexahydropentalen-2-yl acetate. ¹H NMR (600 MHz, CDCl₃) δ 7.66-7.62 (m, 6H), 7.46-7.42 (m, 3H), 7.41-7.37 (m, 6H), 5.82-5.77 (m, 1H), 5.24-5.20 (m, 1H), 3.93 (q, J=8.0 Hz, 1H), 2.98-2.91 (m, 1H), 2.49-2.43 (m, 1H), 2.40-2.32 (m, 1H), 2.20-2.12 (m, 1H), 2.09-2.04 (m, 2H), 1.99 (s, 3H), 1.14-1.08 (m, 1H).

Step 7: To a flask containing allyl palladium(II) chloride dimer (1.66 g, 4.45 mmol), dppf (6.36 g, 11.1 mmol), 4-methyl-7H-pyrrolo[2,3-d]pyrimidine (4.44 g, 33.4 mmol), and potassium tert-butoxide (3.74 g, 33.4 mmol) was added THF (100 mL) under an atmosphere of argon. The solution was stirred at room temperature for 10 minutes. Then a solution of (2R,6S,6aS)-6-((triphenylsilyl)oxy)-1,2,4,5,6,6a-hexahydropentalen-2-yl acetate (9.8 g, 22 mmol) in THF (100 mL) was added, and the reaction was heated to 40° C. overnight. The reaction was cooled to room temperature, filtered through Celite®, and concentrated under reduced pressure. The residue was subjected to column chromatography on silica (10-50% EtOAc/hexanes) to afford 4-methyl-7-((2R,6S,6aS)-6-((triphenylsilyl)oxy)-1,2,4,5,6,6a-hexahydropentalen-2-yl)-7H-pyrrolo[2,3-d]pyrimidine. MS: 514 (M+1). ¹H NMR (600 MHz, CDCl₃) δ 8.75 (s, 1H), 7.65-7.58 (m, 6H), 7.43-7.38 (m, 3H), 7.38-7.31 (m, 6H), 7.05 (d, J=3.7 Hz, 1H), 6.54 (d, J=3.6 Hz, 1H), 6.15 (s, 1H), 5.23-5.19 (m, 1H), 3.99 (q, J=7.9 Hz, 1H), 3.17-3.11 (m, 1H), 2.78 (s, 3H), 2.63-2.58 (m, 1H), 2.49-2.42 (m, 1H), 2.29-2.22 (m, 1H), 2.20-2.12 (m, 2H), 1.22-1.15 (m, 1H).

Step 8: To a flask containing a solution of 4-methyl-7-((2R,6S,6aS)-6-((triphenylsilyl)oxy)-1,2,4,5,6,6a-hexahydropentalen-2-yl)-7H-pyrrolo[2,3-d]pyrimidine (13.2 g, 25.7 mmol) in THF (300 mL) was added water (150 mL). The solution was cooled to 0° C., then NMO (6.02 g, 51.4 mmol) was added, followed by Osmium (VIII) oxide (7.8 mL, 4% in water, 1.3 mmol). The reaction was stirred overnight, and the bath was allowed to expire naturally. The reaction was quenched with saturated aqueous sodium sulfite (60 mL), and stirring was continued at room temperature for 30 minutes. The reaction was poured into a separatory funnel containing water and extracted with 25% IPA/chloroform. The aqueous layer was separated and washed twice more with 25% IPA/chloroform. The combined organic layers were dried over sodium sulfite, filtered over Celite®, and concentrated under reduced pressure to afford crude (1S,2R,3aR,4S,6aR)-2-(4-methyl-7H-pyrrolo[2,3-d]pyrimidin-7-yl)-4-((triphenylsilyl)oxy)hexahydropentalene-1,6a(1H)-diol. This crude product was used directly in the next step.

Step 9: To a flask containing the crude (1S,2R,3aR,4S,6aR)-2-(4-methyl-7H-pyrrolo[2,3-d]pyrimidin-7-yl)-4-((triphenylsilyl)oxy)hexahydropentalene-1,6a(1H)-diol was added DCM (200 mL), followed by 2,2-dimethoxypropane (35 mL, 290 mmol) and p-toluenesulfonic acid monohydrate (17.1 g, 90 mmol). The reaction was stirred at room temperature for 1 h. The reaction was quenched with saturated aqueous sodium bicarbonate (100 mL). The organic layer was separated by Phase Separator and concentrated under reduced pressure. The residue was purified by column chromatography on silica (50-100% EtOAc/hexanes to 100% 3:1 EtOAc:EtOH)) to afford (3aS,4R,5aR,6S,8aR)-2,2-dimethyl-4-(4-methyl-7H-pyrrolo[2,3-d]pyrimidin-7-yl)hexahydro-5H-pentaleno[1,6a-d][1,3]dioxol-6-ol. MS: 330 (M+1).

Intermediate 38: Intermediate 38 in Table 5 was synthesized using the protocol described in Intermediate 37, making the appropriate substitution for 4-methyl-7H-pyrrolo[2,3-d]pyrimidine in step 7. The substituted starting material was commercially acquired, synthesized as reported above, or synthesized through known routes reported in the literature.

TABLE 5

| Intermediate | Structure | Name | MS |
|---|---|---|---|
| 38 | 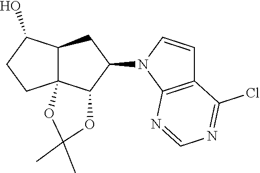 [SMILES: CC1(C)O[C@H]2[C@H](n3ccc4c(Cl)ncnc43)C[C@@H]3[C@@H](O)CC[C@@]32O1] | (3aS,4R,5aR,6S,8aR)-4-(4-chloro-7H- pyrrolo[2,3-d]pyrimidin-7-yl)-2,2- dimethylhexahydro-5H-pentaleno[1,6a- d][1,3]dioxol-6-ol | 350 (M + 1) |

Intermediate 39: Intermediate 39 in Table 6 was synthesized using the protocol described in Intermediate 13, making the appropriate substitution for 4-chloro-5-(difluoromethyl)-7H-pyrrolo[2,3-d]pyrimidine in step 1. The substituted starting material was commercially acquired, synthesized as reported above, or synthesized through known routes reported in the literature.

TABLE 6

| Intermediate | Structure | Name | MS |
|---|---|---|---|
| 39 | 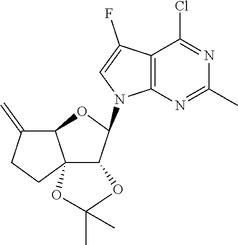 | 4-chloro-7-((3a,4R,5aR,8aR)-2,2- dimethyl-6- methylenehexahydrocyclopenta[2,3]furo [3,4-d][1,3]dioxol-4-yl)-5-fluoro-2- methyl-7H-pyrrolo[2,3-d]pyrimidine | 380 (M + 1) |

### General Synthesis Schemes Applicable for Example 138

### General Synthesis 1A

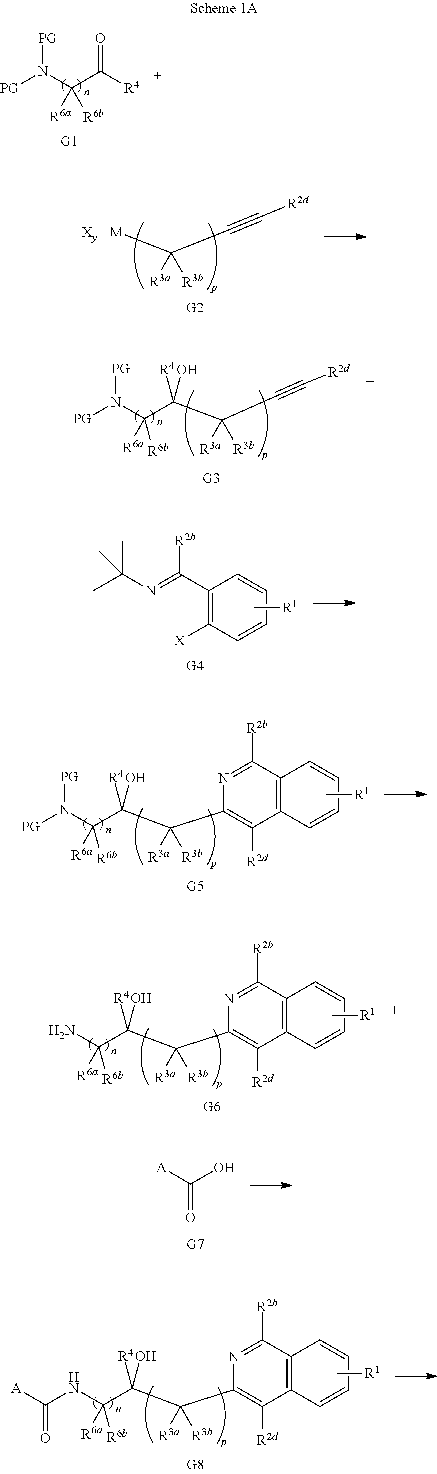

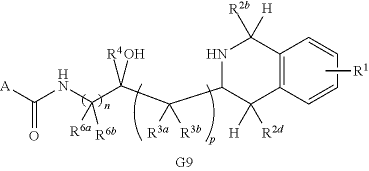

Scheme 1A illustrates the synthesis of compounds with the structure G9. A coupling of a carbonyl compound of structure G1 with an organometallic compound of structure G2 to give a compound with structure G3 will be apparent to those skilled in the art. The group represented by (M) includes but is not limited to Mg, In, Zn and the group represented by (X) may be a halide where (Y) may be the number 1-3. Suitable protected amino groups represented by (PG) include but are not limited to phthalimide, and methods for the removal of said protecting groups are known to those skilled in the art (for example Greene's Protective Groups in Organic Synthesis, 4th Edition). Synthesis of compounds with structure G5 is performed by reacting alkyne G3 with compounds of structure G4 in the presence of atransition metal catalyst or combination of transition metal catalysts such as but not limited to bis(triphenylphosphine)nickel(II) chloride/Zn.

After removal of the protecting group, methods to synthesize amides G8 are apparent to those skilled in the art, and include for example the use of reagents such as HATU, HBTU, T3P and EDCI/HOBt, and the use of activated forms of the carboxylic acid G7 such as the corresponding acyl halide, carbamate or N-hydroxysuccinimide ester. Transformation of isoquinolines of structure G8 to give tetrahydroisoquinolines of structure G9 will be apparent to those skilled in the art and such methods include but are not limited to reduction in the presence of a transition metal catalyst.

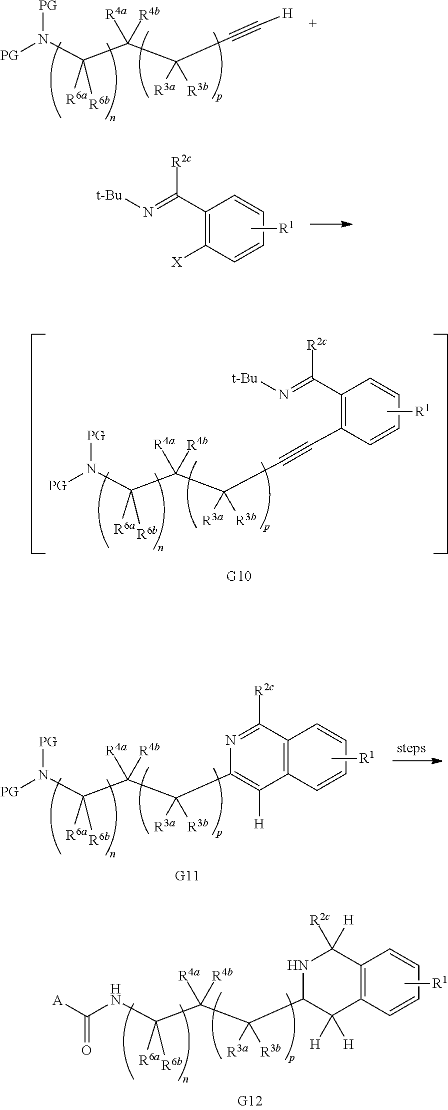

Alternatively, for the synthesis of compounds with structure G12 where R^{2a}, R^{2b}, R^{2d}═H, a coupling of an alkyne and an aryl halide will be apparent to those skilled in the art and such methods include a coupling in the presence of a transition metal catalyst or catalyst combination such as but not limited to PdCl₂(PPh₃)₂/CuI and Pd(OAc)₂/PPh₃. This may be followed by cyclisation either in situ or as a separate step to give isoquinoline of structure G11. The synthetic steps to give compounds with general formula G12 will be similar to those used in Scheme 1A.

### General Synthesis 2A

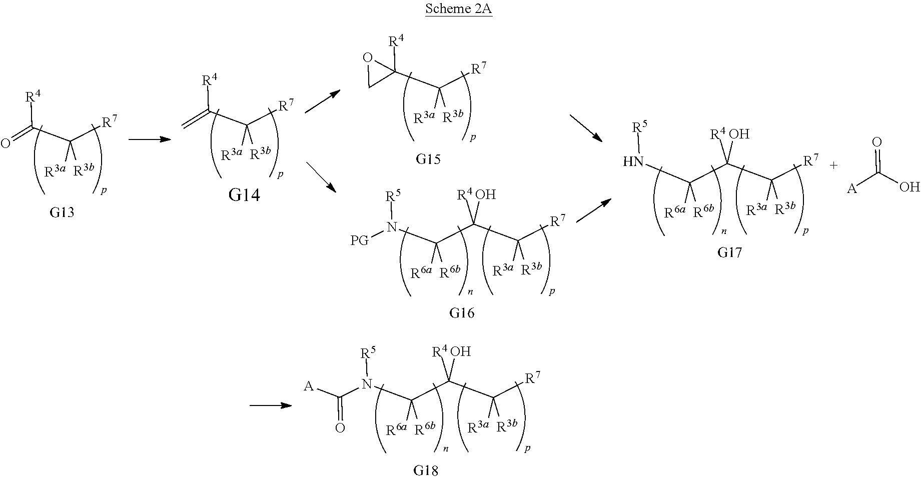

Where R⁷ represents the fused ring group.

Scheme 2A illustrates the synthesis of compounds G18 from aldehyde G13 (or ketone where R⁴=Me). Conversion of a carbonyl to an alkene will be apparent to those skilled in the art but methods include but are not limited to a Wittig reaction with [Ph₃PMe]⁺Br⁻ in the presence of a base such as KHMDS. The alkene G14 can be epoxidised with reagents such as mCPBA and then reacted with an amine to give intermediate G16. Alternatively, an aminohydroxylation can be performed by methods such as but not limited to reaction with (PG)NHOTs in the presence of potassium osmate dihydrate. Removal of the protecting group will be apparent to those skilled in the art (for example Greene's Protective Groups in Organic Synthesis, 4th Edition) and gives intermediate G17. Amide bond formation to give compounds G18 can be performed by methods previously described (General synthesis 1).

### General Synthesis 3A

Scheme 3A illustrates the synthesis of compounds G22 beginning with a Henry reaction between an aldehyde G19 (or ketone where R⁴=Me) and nitromethane in the presence or absence of a suitable base, such as but not limited to DBU, a KF, TBAF or sodium hydroxide, in the presence or absence of a chiral or achiral transition metal compound for example but not limited to complexes of copper, cobalt or zinc to furnish a nitro-alcohol G20. Reduction of the nitro group to the primary amine G21 will be apparent to those skilled in the art and includes but is not limited to using reducing conditions such as a transition metal (Fe, In, Zn) in the presence of HCl, hydrogenation in the presence of a transition metal or transition metal catalyst. Amide bond formation to give compounds G22 can be performed by methods previously described (General synthesis 1). The method can also be carried out with nitroethane and other nitroalkanes, as appropriate.

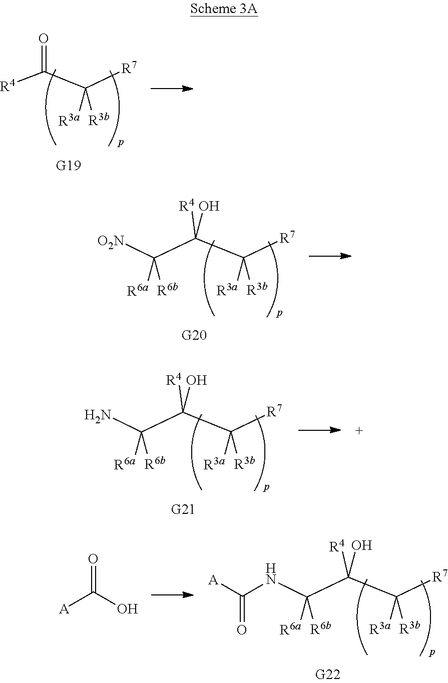

### General Synthesis 4A

Scheme 4A illustrates the addition of an amine (HNR⁸R⁹), as a substituent which is a part of A. This can be achieved by coupling a relevant carboxylic acid to a primary amine or a secondary amine, NHR⁸R⁹. Methods to form such amides will be apparent to those skilled in the art, but include for example the use of reagents such as HATU, HBTU, T3P and EDCI/HOBt, and the use of activated forms of the carboxylic acid such as the corresponding acyl halide, mixed anhydride or N-hydroxysuccinimide ester. The group denoted by (X) may be but not limited to halogen, tosylate or other suitable group. Conversion of (X) in G22 into an ester in G23 will be apparent to those skilled in the art, but include for example a carbonylation reaction which can be achieved by the use of carbon monoxide in the presence of a transition metal catalyst such as but not limited to PdCl₂dppfDCM; and an alcoholic solvent such as but not limited to methanol, ethanol, isopropanol or tert-butyl alcohol. Formation of the carboxylic acid can be achieved by for example hydrolysis with a base such as an alkali metal hydroxide or an acid for example aqueous hydrochloric acid to form G24. The amide formation to form G25 can be achieved by the methods outline in Scheme 1A.

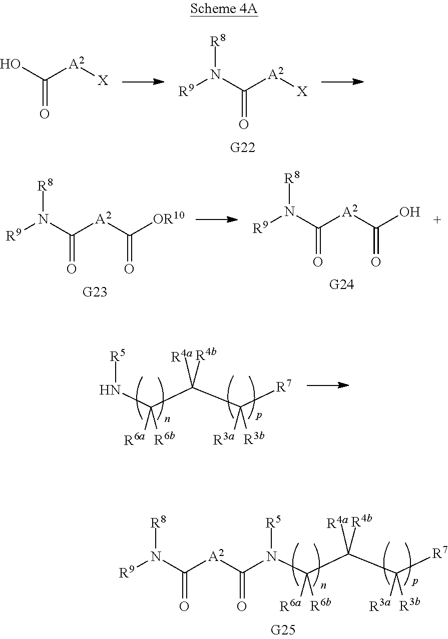

Alternatively, for the synthesis of ester G24 the order of steps can be reversed as described in Scheme 4B.

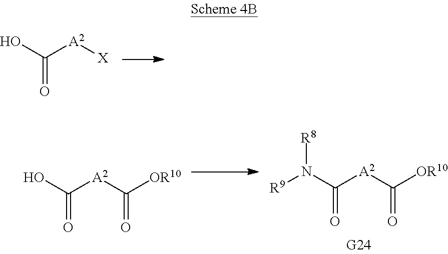

Alternatively, for the synthesis of amide G25 the steps may be reordered such that the formation of the R⁸R⁹N amide on the A substituent occurs after the coupling of A to the primary amine G21. This may be achieved by coupling a suitable amine with an intermediate where A bears a suitable functional group for coupling, for example but not limited to a carboxylic acid or alkali metal carboxylate salt, as shown in Scheme 4C.

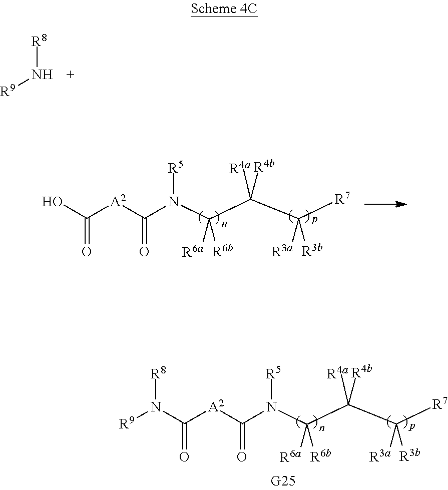

### General Synthesis 5A

Scheme 5A illustrates the addition of an R¹¹ group, as a substituent which is part of A. This can be achieved using any suitable coupling reaction known to the person skilled in the art, for example by Suzuki coupling. The groups denoted by R¹¹X and B¹ are chosen to be suitable for the coupling reaction employed. For example, in the case of a Suzuki coupling reaction (X) may be a halogen, tosylate or other suitable group and B¹ represents a suitable boron compound including, but not limited to, a boronic acid or boronic ester.

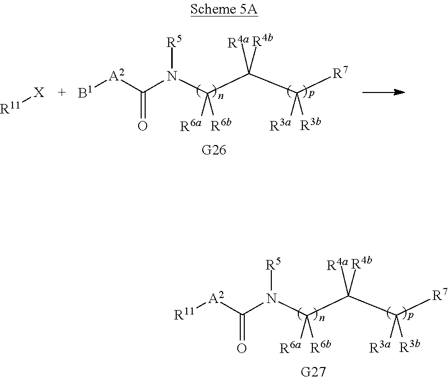

Examples of B¹ that can be used in the Suzuki coupling include, but are not limited to, those shown below.

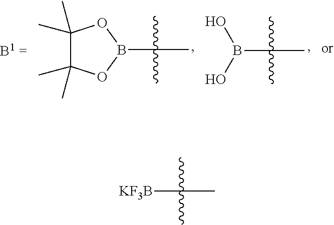

The types of R¹¹X compounds that can be used in the Suzuki coupling include, but are not limited to:

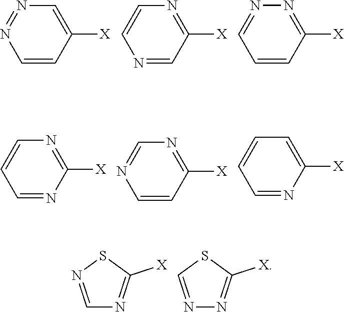

In addition to scheme 5A, the position of the (X) and (B¹) can be reversed as shown below in scheme 2B, to give the same final compound G27. Similarly to Scheme 2A, the groups denoted by R¹¹B¹ and (X) are chosen to be suitable for the coupling reaction employed. For example, in the case of a Suzuki coupling reaction (X) may be a halogen, tosylate or other suitable group and R¹¹B¹ represents a suitable boron compound including, but not limited to, a boronic acid or boronic ester.

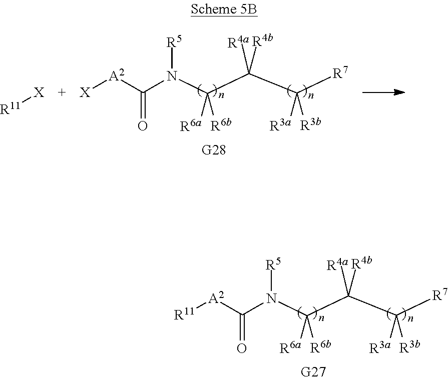

The types of R¹¹B¹ compounds that can be used in the Suzuki coupling include, but are not limited to:

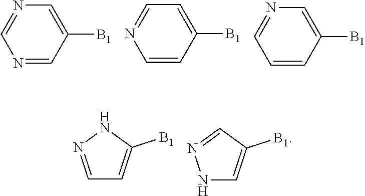

A variety of coupling reactions may be used to introduce the R¹¹ group other than Suzuki coupling, such as for example transition metal catalysed coupling reactions of for example tin (Stille type reaction) and zinc (Negishi type reaction) compounds. Substitution of the halogen by suitable nucleophiles in the presence or absence of other reagents such as for example transition metal compounds is also suitable.

Coupling reactions can also be used to prepare the carboxylic acids used in Scheme 1A for the amide formations, scheme 5C. In starting material G30 and G32, A as described herein, consists of -A²X and -A²B¹ respectively. In the product G33, A as described herein, consists of -A²R¹¹. The groups denoted by (X) and B¹ are chosen to be suitable for the coupling reaction employed. For example, in the case of a Suzuki coupling reaction (X) may be a halogen, tosylate or other suitable group and B¹ represents a suitable boron compound including, but not limited to, a boronic acid or boronic ester.

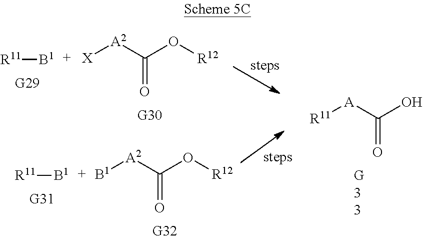

In G30 and G32 R¹² can be a H or a carbon group for example but not limited to Me, Et, Pr, iPr, Bu, t-Bu. In these instances where R¹² is carbon group it may be necessary to form the carboxylic acid before use in the amide coupling (Scheme 1A), generally this can be achieved by for example hydrolysis with a base such as an alkali metal hydroxide or an acid for example aqueous hydrochloric acid to form G33. The same method for converting an ester to a carboxylic acid is used in other general schemes.

### General Synthesis 6A

Scheme 6A illustrates the addition of an R¹³ group, as a substituent which is part of A. This can be achieved using any suitable coupling reaction known to the person skilled in the art, for example, by an SnAr displacement or Buchwald coupling. The group denoted by (X) may be but not limited to halogen and is chosen to be suitable for the coupling reaction employed.

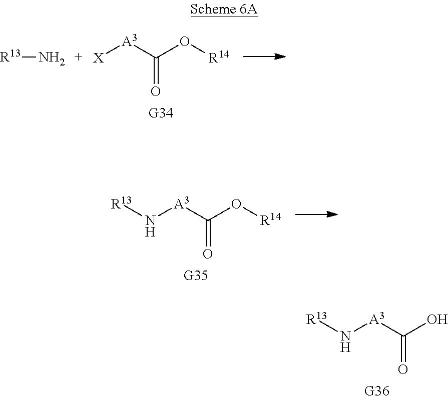

In G34 and G35 R¹⁴ can be a H or a carbon group for example but not limited to Me, Et, Pr, iPr, Bu, t-Bu. In these instances, it may be necessary to form the carboxylic acid before use in an amide coupling (Scheme 1A), generally this can be achieved by, for example, hydrolysis with a base such as an alkali metal hydroxide or an acid, for example, aqueous hydrochloric acid to form G36. The same method for converting an ester to a carboxylic acid is used in other general schemes.

This method may also be extended to the addition of secondary amines.

Alternatively, to synthesise ether linked compounds, a similar strategy can be employed as shown in Scheme 6B. This can be achieved using any suitable coupling reaction known to a person skilled in the art, for example, by an SnAr displacement or an Ullman-type coupling to give compounds with structure G37. Upon hydrolysis using methods previously described, compounds with structure G38 may be obtained and used in an amide bond formation as shown in scheme 1A.

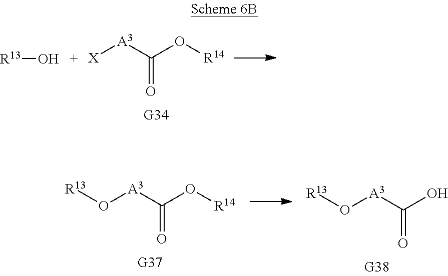

Both the above couplings may also be reversed, such that the group added is R¹³—X.

### Synthesis of Intermediates Applicable for Example 138

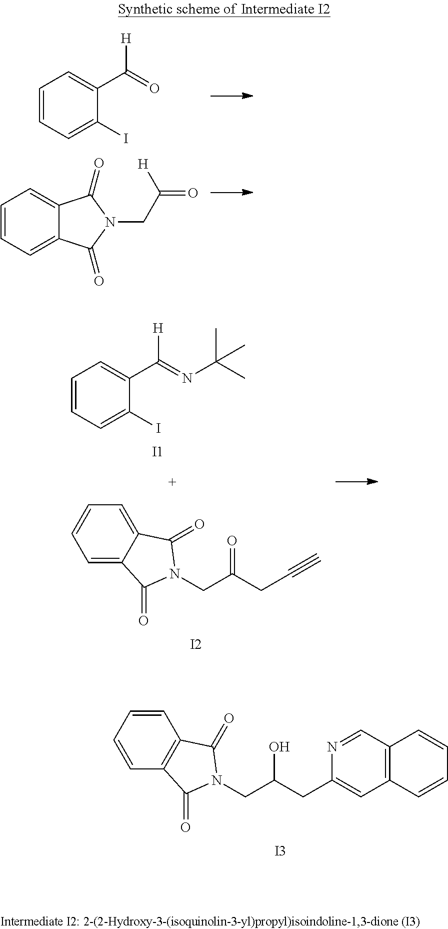

### (a) (E)-N-tert-Butyl-1-(2-iodophenyl)methanimine (I1)

2-Iodobenzaldehyde (1.460 g, 6.29 mmol), water (3 mL) and tert-butylamine (1.98 mL, 18.9 mmol) were stirred at room temperature for 18 hours. The volatiles were removed in vacuo and the residue was extracted with diethyl ether (2×5 mL). The combined ether extracts were dried over sodium sulfate and concentrated to give the desired compound as an oil (1.45 g, 80%)

### (b) 2-(2-Hydroxypent-4-yn-1-yl)isoindoline-1,3-dione (I2)

2-(1,3-Dioxoisoindolin-2-yl)acetaldehyde (1.00 g, 5.29 mmol), indium powder (1.21 g, 10.6 mmol), THF (10 mL), water (10 mL) and propargyl bromide (80% in toluene, 1.38 mL, 10.6 mmol) were stirred vigorously at room temperature. After four hours, the volatile solvents were removed in vacuo and the aqueous residue diluted with water (100 mL). The aqueous mixture was extracted with DCM (3×100 mL) and the combined organic extracts were washed with brine (100 mL), dried over sodium sulfate and concentrated. Chromatography (40 g silica cartridge, 0-60% ethyl acetate in petroleum benzine 40-60° C.) gave the desired compound as a solid (531 mg, 44%)¹H NMR (400 MHz, CDCl₃) δ 7.90-7.84 (m, 2H), 7.77-7.71 (m, 2H), 4.15-4.07 (m, 1H), 3.93-3.89 (m, 2H), 2.69 (br s, 1H), 2.58-2.43 (m, 2H), 2.09 (t, J=2.7 Hz, 1H); LCMS-B 3.39 min; m/z 212.1 [M−H₂O+H]⁺; 230.1 [M+H]⁺; 252.1 [M+Na]⁺.

### (c) 2-(2-Hydroxy-3-(isoquinolin-3-yl)propyl)isoindoline-1,3-dione (I3)

A Schlenk tube was loaded with zinc dust (86 mg, 1.3 mmol) and bis(triphenylphosphine)nickel(II) chloride (21 mg, 5 mol %) and purged with nitrogen. A solution of (E)-N-tert-butyl-1-(2-iodophenyl)methanimine 11 (188 mg, 0.65 mmol) and 2-(2-Hydroxypent-4-yn-1-yl)isoindoline-1,3-dione 12 (150 mg, 0.65 mmol) in dry acetonitrile (15 mL) was added via cannula and the mixture stirred at 80° C. under nitrogen. After 30 minutes, the mixture was cooled to room temperature, filtered through Celite and the Celite washed with acetonitrile (30 mL). The combined filtrates were concentrated in vacuo and the material was subjected to column chromatography (12 g silica cartridge, 0-10% methanol/DCM then 100% methanol). The mixture was loaded in methanol onto a 10 g SCX cartridge, the cartridge washed with methanol (60 mL) and eluted with 2.0 M ammonia in methanol (100 mL). The basic eluate was concentrated in vacuo to a sticky solid. The residue was slurried in ethyl acetate (2×2 mL) and the insoluble material dried in vacuo to give the desired compound (116 mg, 53% yield) as a solid. ¹H NMR (400 MHz, d₆-DMSO) δ 9.22-9.17 (m, 1H), 8.06-8.01 (m, 1H), 7.88-7.84 (m, 1H), 7.83-7.76 (m, 4H), 7.74-7.69 (m, 1H), 7.65 (s, 1H), 7.62-7.56 (m, 1H), 5.10 (d, J=5.5 Hz, 1H), 4.43-4.31 (m, 1H), 3.76-3.65 (m, 1H), 3.63-3.54 (m, 1H), 3.07-2.90 (m, 2H); LCMS-B: Retention Time 3.26 min; m/z 333.2 [M+H]⁺.

### (ii) 2-(2-Hydroxy-2-(isoquinolin-3-yl)ethyl)isoindoline-1,3-dione (15)

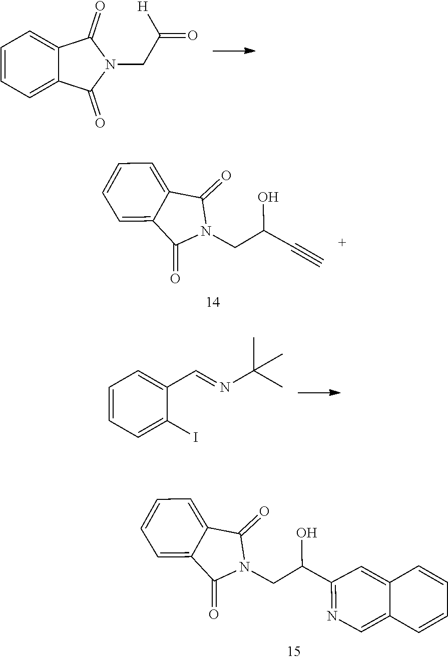

### (a) 2-(2-Hydroxybut-3-yn-1-yl)isoindoline-1,3-dione (I4)

A 0.5 M THF solution of ethynyl magnesium bromide (3.33 mL, 1.67 mmol) was cooled to 0° C. under nitrogen. A solution of 2-(1,3-dioxoisoindolin-2-yl)acetaldehyde (300 mg, 1.59 mmol) in THF (3 mL) was added cannula and the mixture stirred at 0° C. under nitrogen. After 1.5 hours, the mixture was quenched with a saturated aqueous solution of ammonium chloride (3 mL) and the volatile solvents were removed in vacuo. The residue was diluted with water (10 mL) and DCM (15 mL), the aqueous phase was extracted with DCM (2×15 mL). The combined organic phases were dried over sodium sulfate and concentrated. Chromatography (12 g silica cartridge, 0-60% ethyl acetate in petroleum benzine 40-60° C.) gave the desired compound as a solid (142 mg, 42%). ¹H NMR (400 MHz, CDCl₃) δ 7.88 (dd, J=5.5, 3.1 Hz, 2H), 7.75 (dd, J=5.5, 3.1 Hz, 2H), 4.74-4.66 (m, 1H), 4.08 (dd, J=14.3, 8.0 Hz, 1H), 3.94 (dd, J=14.2, 3.9 Hz, 1H), 2.83 (d, J=7.7 Hz, 1H), 2.49 (d, J=2.1 Hz, 1H); LCMS-B: Retention Time 3.37 min; no product ions detected.

### (b) 2-(2-Hydroxy-2-(isoquinolin-3-yl)ethyl)isoindoline-1,3-dione (15)

A Schlenk tube was loaded with zinc dust (84 mg, 1.3 mmol) and bis(triphenylphosphine)nickel(II) chloride (21 mg, 5 mol %) then flushed with nitrogen. A solution of N-tert-butyl-1-(2-iodophenyl)methanimine (184 mg, 0.641 mmol) and 2-(2-hydroxybut-3-yn-1-yl)isoindoline-1,3-dione 14 (138 mg, 0.641 mmol) in dry acetonitrile (15 mL) was added via cannula, and the mixture stirred at 80° C. under nitrogen. After 30 minutes, the mixture was cooled and filtered through Celite. The Celite was washed with acetonitrile (2×20 mL) and the combined filtrates concentrated. Chromatography (12 g silica cartridge, 0-10% methanol/DCM) gave the desired compound as a solid (174 mg, 85%). ¹H NMR (400 MHz, d₄-DMSO) δ 9.20 (s, 1H), 8.15-8.07 (m, 1H), 8.02-7.97 (m, 1H), 7.96 (s, 1H), 7.91-7.80 (m, 5H), 7.80-7.73 (m, 1H), 7.68-7.61 (m, 1H), 5.91 (d, J=4.9 Hz, 1H), 5.18-5.07 (m, 1H), 4.04-3.95 (m, 1H), 3.92-3.79 (m, 1H); LCMS-B: Retention Time 3.41 min; m/z 319.1 [M+H]⁺.

### (iii) 4-(Morpholine-4-carbonyl)benzoic acid (I7)

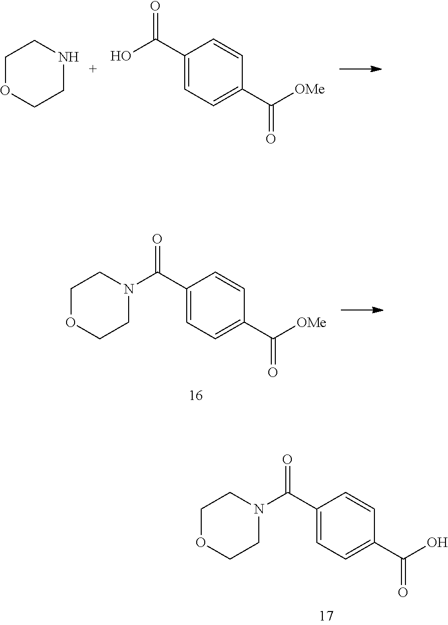 [SMILES: C1COCCN1 ; COC(=O)c1ccc(C(=O)O)cc1 ; COC(=O)c1ccc(C(=O)N2CCOCC2)cc1 ; O=C(O)c1ccc(C(=O)N2CCOCC2)cc1]

### (a) Methyl 4-(morpholine-4-carbonyl)benzoate (I6)

To an ice-cooled (0° C.) solution of mono-methyl terephthalate (10.0 g, 55.5 mmol) in DCM (100 mL) was added oxalyl chloride (5.7 mL, 67 mmol) and a catalytic amount of DMF (10 drops). The mixture was stirred at 0° C. for 3 hours and the solvent was removed in vacuo. The residue was taken up in DCM (100 mL), cooled to 0° C. and morpholine (5.3 mL, 61 mmol) was added drop-wise followed by triethylamine (9.3 mL, 67 mmol). The reaction was stirred at room temperature for 16 hours. The reaction mixture was diluted with a saturated aqueous solution of NaHCO₃ (100 mL). The aqueous layer was extracted with DCM (3×100 mL) and the combined organic layers were washed with 1 M HCl (100 mL), water (100 mL) and brine (25 mL). The organic layer was dried (Na₂SO₄), filtered and concentrated in vacuo give the desired compound (14.0 g, quantitative) as a solid: ¹H NMR (400 MHz, CDCl₃) δ 8.09 (d, J=8.6 Hz, 2H), 7.47 (d, J=8.6 Hz, 2H), 3.93 (s, 3H), 3.87-3.71 (m, 4H), 3.69-3.52 (m, 2H), 3.50-3.25 (m, 2H) COOH peak not observed; LCMS-B: Retention Time 3.35 min, m/z 250 [M+H]⁺.

### (b) 4-(Morpholine-4-carbonyl)benzoic acid (I7)

(i) A suspension of methyl 4-(morpholine-4-carbonyl)benzoate I6 (5.00 g, 20.1 mmol) and LiOH.H₂O (926 mg, 22.1 mmol) in THF (100 mL), MeOH (10 mL) and water (2 mL) was stirred at room temperature for 16 hours. The volatiles were concentrated under reduced pressure and the resulting gum suspended in a 0.5 M aqueous citric acid solution (100 mL). Et₃N (1 mL) was added and the water layer extracted with DCM (3×100 mL). The combined organic fractions were dried over MgSO₄ and the volatiles removed in vacuo to give the product as a solid (3.55 g, 75%): ¹H NMR (400 MHz, Chloroform-d) δ 8.17 (d, J=8.5, 2H), 7.53 (d, J=8.5, 2H), 3.95-3.58 (m, 6H), 3.44 (s, 2H); LCMS-B: Retention Time 3.162 min; m/z 236.2 [M+H]⁺. (ii) Methyl 4-(morpholine-4-carbonyl)benzoate (I6) (6.95 g, 27.9 mmol) was dissolved in THF (100 mL) and a solution of lithium hydroxide monohydrate (1.65 g, 39.4 mmol) in water (50 mL) was added. The mixture was stirred at room temperature for 2 hours. The volatile solvents were removed in vacuo and the aqueous residue diluted with water (50 mL) and 10% w/v aqueous sodium hydrogen sulfate monohydrate (50 mL). The resulting slurry was filtered, the collected solid washed with water (20 mL) and dried under vacuum to give the desired compound (5.77 g, 88% yield) as a powder. LCMS-B: Retention Time 3.17 min; m/z 236.1 [M+H]⁺, 258.2 [M+Na]; m/z 234.1 [M−H]⁻

### (iv) tert-Butyl (S)-3-((R)-2-amino-1-hydroxyethyl)-3,4-dihydroisoquinoline-2(1H)-carboxylate (I12)

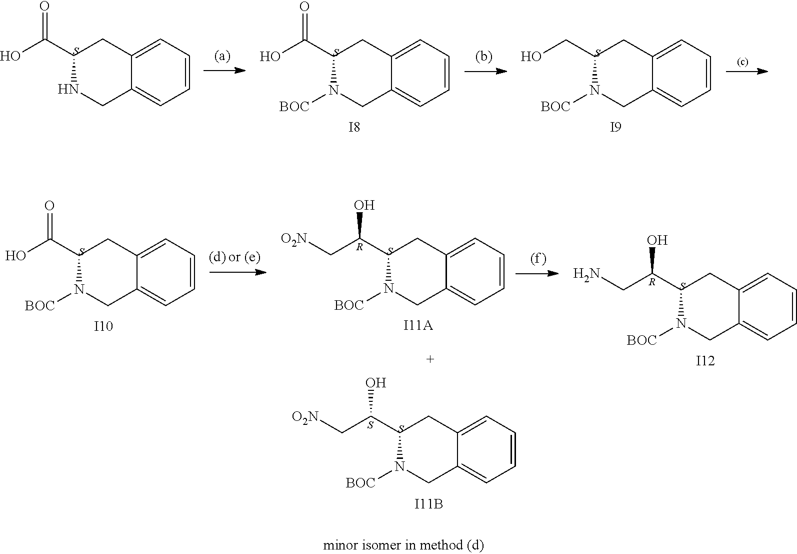

### (a) (S)-2-(tert-Butoxycarbonyl)-1,2,3,4-tetrahydroisoquinoline-3-carboxylic acid (I8)

(S)-1,2,3,4-Tetrahydroisoquinoline-3-carboxylic acid (5.00 g, 28.2 mmol) was vigorously stirred in 1,4-dioxane (100 mL) and water (50 mL). Sodium bicarbonate (4.74 mg, 56.4 mmol) and Boc anhydride (6.77 g, 31.0 mmol) were added and the mixture was stirred vigorously at room temperature. After 17 hours the mixture was concentrated in vacuo and the residue dissolved in water (200 mL). A 30% w/v aqueous solution of sodium hydrogen sulfate monohydrate (30 mL) was added and the mixture extracted with chloroform (3×200 mL). The pooled organic extracts were washed with brine, dried over sodium sulfate and concentrated in vacuo to give the desired compound (7.50 g, 96% yield) as a thick syrup. LCMS-B: Retention Time 3.64 min; m/z 178.1 [M-Boc+2H]⁺; m/z 276.1 [M−H]⁻

### (b) tert-Butyl (S)-3-(hydroxymethyl)-3,4-dihydroisoquinoline-2(1H)-carboxylate (I9)

(S)-2-(tert-Butoxycarbonyl)-1,2,3,4-tetrahydroisoquinoline-3-carboxylic acid (I8) (7.50 g, 27.0 mmol) was dissolved in THF (150 mL) and CDI (8.77 g, 54.1 mmol) was added. The mixture was stirred for 30 minutes at room temperature then cooled to 0° C. A solution of sodium borohydride (1.16 g, 30.5 mmol) in water (15 mL) was added dropwise. After 40 minutes the mixture was quenched with acetone (25 mL) and concentrated in vacuo. The residue was partitioned between water (250 mL) and ethyl acetate (200 mL). The separated aqueous phase was extracted with ethyl acetate (2×250 mL), the combined organic extracts washed with 5% w/v aqueous NaHSO₄ (250 mL), brine (200 mL), dried over sodium sulfate and concentrated in vacuo. The residue was loaded in diethyl ether (50 mL) onto a plug of basic alumina and silica (50 mL each). The plug was eluted with diethyl ether (250 mL) and the eluate evaporated to give the desired compound (5.93 g, 83% yield) as a syrup. ¹H NMR (400 MHz, CDCl₃) δ 7.25-7.06 (m, 4H), 4.82-4.59 (m, 1H), 4.57-4.38 (m, 1H), 4.37-4.19 (m, 1H), 3.57-3.40 (m, overlaps with trace solvent), 3.03 (dd, J=16.1, 5.7 Hz, 1H), 2.80 (d, J=16.1 Hz, 1H), 1.50 (s, 9H). LCMS-B: Retention Time 3.66 min; m/z 164.2 [M-Boc+2H]⁺, 286.2 [M+Na]⁺

### (c) tert-Butyl (S)-3-formyl-3,4-dihydroisoquinoline-2(1H)-carboxylate (I10)

tert-Butyl (S)-3-(hydroxymethyl)-3,4-dihydroisoquinoline-2(1H)-carboxylate (I9) (1.50 g, 5.70 mmol), DCM (25 mL) and DMSO (5 mL) were cooled to 0° C. Triethylamine (2.38 mL, 17.1 mmol) was added, followed by pyridine-sulfur trioxide complex (2.72 g, 17.1 mmol). The mixture was stirred at 0° C. for 10 minutes then allowed to come to room temperature. After 2 hours, saturated sodium bicarbonate (75 mL) and water (75 mL) were added, and the mixture extracted with diethyl ether (3×150 mL). The pooled ether extracts were washed with 1:1 water: saturated aqueous NH₄Cl (200 mL), brine (200 mL), dried over sodium sulfate and concentrated in vacuo to give the desired compound as an oil which was used without further purification.

### (d) tert-Butyl (S)-3-((R)-1-hydroxy-2-nitroethyl)-3,4-dihydroisoquinoline-2(1H)-carboxylate (I11A) and tert-butyl (S)-3-((S)-1-hydroxy-2-nitroethyl)-3,4-dihydroisoquinoline-2(1H)-carboxylate (I11B)

A solution of tert-butyl (S)-3-formyl-3,4-dihydroisoquinoline-2(1H)-carboxylate (I10) (5.70 mmol @ 100% conversion) in i-propanol (50 mL) was cooled to 0° C. Nitromethane (1.22 mL, 22.8 mmol) and potassium fluoride (331 mg, 5.70 mmol) were added and the mixture stirred for 18 hours, allowing the temperature to come to room temperature as the ice bath thawed. The mixture was diluted with water (200 mL) and extracted with DCM (3×200 mL). The pooled DCM extracts were washed with brine, dried over sodium sulfate and concentrated in vacuo. Chromatography (40 g silica cartridge, 0-20% ethyl acetate/hexanes) gave two partly overlapping peaks, which were split into early (11A major, syrup, 697 mg, 37% yield) and late (11B minor, syrup, 170 mg, 9% yield) fractions. Overall: 867 mg, 47% yield.

Data for major isomer tert-butyl (S)-3-((R)-1-hydroxy-2-nitroethyl)-3,4-dihydroisoquinoline-2(1H)-carboxylate I11A:

¹H NMR (400 MHz, d₄-MeOD) δ 7.25-7.14 (m, 4H), 4.85-4.49 (m, 5H), 4.44 (dd, J=12.6, 9.3 Hz, 1H), 4.37-3.99 (m, overlaps with solvent), 3.19 (dd, J=15.9, 3.2 Hz, 1H), 2.92 (dd, J=15.8, 5.6 Hz, 1H), 1.51 (s, 9H). LCMS-B: Retention Time 3.71 min; m/z 223.2 [M-Boc+2H]⁺, 345.2 [M+Na]⁺; m/z 321.2 [M−H]⁻

Data for minor isomer tert-butyl (S)-3-((S)-1-hydroxy-2-nitroethyl)-3,4-dihydroisoquinoline-2(1H)-carboxylate I11B:

¹H NMR (400 MHz, d₄-MeOD) δ 7.25-7.11 (m, 4H), 4.75 (d, J=16.5 Hz, 1H), 4.68-4.48 (m, 4H), 4.42-4.23 (m, overlaps with residual nitromethane), 3.06 (dd, J=16.3, 6.1 Hz, 1H), 2.91 (d, J=16.1 Hz, 1H), 1.50 (s, 9H). LCMS-B: Retention Time 3.70 min; m/z 223.2 [M-Boc+2H]⁺, 345.2 [M+Na]⁺; m/z 321.2 [M−H]⁻

### (e) tert-Butyl (S)-3-((R)-1-hydroxy-2-nitroethyl)-3,4-dihydroisoquinoline-2(1H)-carboxylate (I11A)

### Copper Catalyst Used

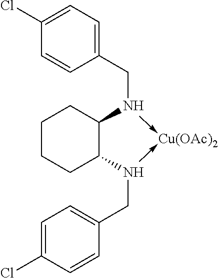

tert-Butyl (S)-3-formyl-3,4-dihydroisoquinoline-2(1H)-carboxylate (I10) (1.9 mmol @100% conversion), absolute ethanol (5 mL), nitromethane (1.02 mL, 19.0 mmol), and the copper catalyst (91 mg, 10 mol %) (see above figure, prepared according to Tetrahedron: Asymmetry (2008) 2310-2315) were stirred at room temperature. After 90 hours the mixture was concentrated in vacuo, chromatography (40 g silica cartridge, 0-15% ethyl acetate/hexanes) gave the desired compound (352 mg, 58% yield over two steps). ¹H NMR (400 MHz, d₄-MeOD) δ 7.25-7.13 (m, 4H), 4.85-4.68 (m, 1H), 4.65-4.49 (m, 1H), 4.49-4.39 (m, 1H), 4.36-3.96 (m, overlaps with trace solvent), 3.19 (dd, J=15.9, 3.2 Hz, 1H), 2.92 (dd, J=15.9, 5.6 Hz, 1H), 1.51 (s, 9H). LCMS-B: Retention Time 3.25 min; m/z 321.1 [M−H]⁻

### (f) tert-Butyl (S)-3-((R)-2-amino-1-hydroxyethyl)-3,4-dihydroisoquinoline-2(1H)-carboxylate (I12)

tert-Butyl (S)-3-((R)-1-hydroxy-2-nitroethyl)-3,4-dihydroisoquinoline-2(1H)-carboxylate (I11A) (1.54 g, 4.78 mmol), absolute ethanol (75 mL) and 10% Pd/C (53% wetted with water, 1.5 g) were stirred under hydrogen (balloon). After 3 hours the mixture was filtered through Celite, the Celite was washed with absolute ethanol (100 mL) and the combined filtrates concentrated in vacuo to give the desired compound (1.34 g, 96% yield) as a syrup. LCMS-B: Retention Time 3.27 min, m/z 293.2 [M+H]⁺

Alternate Synthesis Method

### (a) (S)-2-(tert-Butoxycarbonyl)-1,2,3,4-tetrahydroisoquinoline-3-carboxylic acid (I8)

(S)-1,2,3,4-Tetrahydroisoquinoline-3-carboxylic acid (50.0 g, 282 mmol) was vigorously stirred in a mixture of 1,4-dioxane (1000 mL) and water (500 mL). Sodium bicarbonate (47.4 g, 564 mmol) and Boc anhydride (67.7 g, 310 mmol) were added and the reaction was stirred vigorously at room temperature for 6 days. The mixture was concentrated in vacuo and the residue dissolved in water (2000 mL). A 30% w/v aqueous solution of sodium hydrogen sulfate monohydrate (300 mL) was added and the mixture extracted with chloroform (3×1000 mL). The pooled organic extracts were washed with brine, dried over sodium sulfate and concentrated in vacuo to give the desired compound (90.0 g, quantitative) as a thick syrup. LCMS-B: Retention Time 3.64 min; m/z 178.1 [M-Boc+2H]⁺; m/z 276.1 [M−H]⁻

### (b) tert-Butyl (S)-3-(hydroxymethyl)-3,4-dihydroisoquinoline-2(1H)-carboxylate (I9)

(S)-2-(tert-Butoxycarbonyl)-1,2,3,4-tetrahydroisoquinoline-3-carboxylic acid (I8) (54.0 g, 195 mmol) was dissolved in THF (1000 mL) and CDI (63.2 g, 390 mmol) was added. The mixture was stirred for 2 hours at 30° C. then cooled to 0° C. A solution of sodium borohydride (14.7 g, 390 mmol) in water (120 mL) was added dropwise. After 3 hours the mixture was quenched with acetone (300 mL) and concentrated in vacuo. The residue was partitioned between water (1000 mL) and ethyl acetate (1000 mL). The separated aqueous phase was extracted with ethyl acetate (4×500 mL) and the combined organic extracts washed with 5% w/v aqueous NaHSO₄ (1000 mL), brine (500 mL), dried over sodium sulfate and concentrated in vacuo. The residue was purified by chromatography (5-20% ethyl acetate/petroleum ether) to give the desired compound (30.4 g, 59% yield) as a syrup. ¹H NMR (400 MHz, CDCl₃) δ 7.25-7.06 (m, 4H), 4.82-4.59 (m, 1H), 4.57-4.38 (m, 1H), 4.37-4.19 (m, 1H), 3.57-3.40 (m, overlaps with trace solvent), 3.03 (dd, J=16.1, 5.7 Hz, 1H), 2.80 (d, J=16.1 Hz, 1H), 1.50 (s, 9H). LCMS-B: Retention Time 3.66 min; m/z 164.2 [M-Boc+2H]⁺, 286.2 [M+Na]⁺

### (c) tert-Butyl (S)-3-formyl-3,4-dihydroisoquinoline-2(1H)-carboxylate (I10)

tert-Butyl (S)-3-(hydroxymethyl)-3,4-dihydroisoquinoline-2(1H)-carboxylate (I9) (16 g, 0.06 mol), DCM (250 mL) and DMSO (75 mL) were cooled to 0° C. Triethylamine (25.1 mL, 0.18 mol) was added, followed by pyridine-sulfur trioxide complex (28.6 g, 0.18 mol). The mixture was stirred at 0° C. for 30 minutes then allowed to come to room temperature and stirred at room temperature overnight. Saturated sodium bicarbonate (200 mL) and water (200 mL) were added, and the mixture extracted with diethyl ether (3×300 mL). The pooled ether extracts were washed with 1:1 water:saturated aqueous NH₄Cl (200 mL), dried over sodium sulfate and concentrated in vacuo to give the desired compound (16.0 g) as oil which was used without further purification.

### (e) tert-Butyl (S)-3-((R)-1-hydroxy-2-nitroethyl)-3,4-dihydroisoquinoline-2(1H)-carboxylate (I111A)

To a solution of tert-butyl (S)-3-formyl-3,4-dihydroisoquinoline-2(1H)-carboxylate (I10) (0.06 mol @100% conversion) in absolute ethanol (50 mL) was added a solution of the copper catalyst (6.8 g, 20 mol %) (see above figure, prepared according to Tetrahedron: Asymmetry (2008) 2310-2315) in absolute ethanol (10 mL). The mixture was cooled to 0° C. and nitromethane (36.0 g, 0.6 mol) was added. The reaction was stirred at 0° C. for 3 days, the mixture was concentrated in vacuo and purified by chromatography (5% ethyl acetate/petroleum ether) to give the desired compound (7.5 g, 39% yield over two steps). ¹H NMR (400 MHz, d₄-MeOD) δ 7.25-7.13 (m, 4H), 4.85-4.68 (m, 1H), 4.65-4.49 (m, 1H), 4.49-4.39 (m, 1H), 4.36-3.96 (m, overlaps with trace solvent), 3.19 (dd, J=15.9, 3.2 Hz, 1H), 2.92 (dd, J=15.9, 5.6 Hz, 1H), 1.51 (s, 9H). LCMS-B: Retention Time 3.25 min; m/z 321.1 [M−H]⁻

### (f) tert-Butyl (S)-3-((R)-2-amino-1-hydroxyethyl)-3,4-dihydroisoquinoline-2(1H)-carboxylate (I12)

To a solution of tert-butyl (S)-3-((R)-1-hydroxy-2-nitroethyl)-3,4-dihydroisoquinoline-2(1H)-carboxylate (I11A) (7.5 g, 23.3 mmol) in absolute ethanol (100 mL) was added 10% Pd/C (7.5 g) and the reaction was stirred under an atmosphere of hydrogen. After 3 hours, the mixture was filtered through Celite, the Celite was washed with absolute ethanol (200 mL) and the combined filtrates concentrated in vacuo to give the desired compound (5.3 g, 78% yield) as a solid. LCMS-B: Retention Time 3.27 min, m/z 293.2 [M+H]⁺

### (v) 2-Methylbenzoic acid (I13)

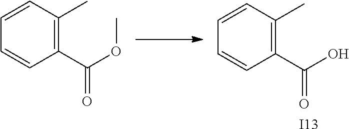

### (a) 2-Methylbenzoic acid (I13)

Methyl 2-methylbenzoate (0.19 mL, 1.33 mmol) was dissolved in THF (4 mL) and water (0.6 mL) and lithium hydroxide monohydrate (0.34 g, 7.99 mmol) was added. The reaction was stirred at room temperature for 24 hours. The volatiles were removed in vacuo and the resulting residue diluted with EtOAc (50 mL) followed by 2 M aqueous NaOH (50 mL). The layers were separated, and the aqueous layer was acidified with 1 M aqueous HCl (checked by pH paper), then extracted with EtOAc (2×70 mL), washed with brine, dried (Na₂SO₄), filtered and concentrated in vacuo to give the desired compound (0.010 g, 6% yield) as a solid. ¹H NMR (400 MHz, CDCl₃) δ 8.05 (dd, J=8.1, 1.5 Hz, 1H), 7.46 (td, J=7.5, 1.5 Hz, 1H), 7.32-7.26 (m, 2H), 2.66 (s, 3H), OH not observed. LCMS-A: Retention Time 5.627 min; mass ion not detected

### (vi) 3-(Pyridazin-4-yl)benzoic acid (I15)

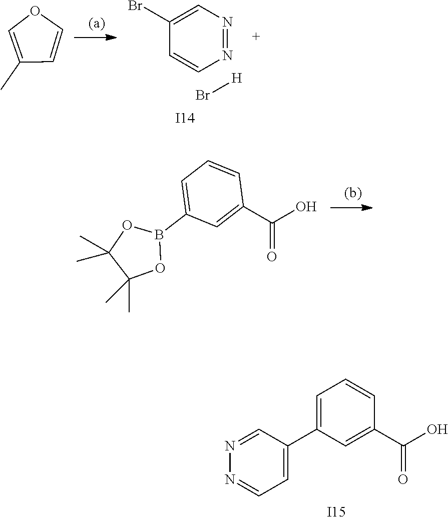

### (a) 4-Bromopyridazine hydrobromide (I14)

Potassium acetate (7.4 g, 75 mmol) and 3-bromofuran (4.0 g, 27 mmol) were stirred in acetic acid (20 mL) and a solution of bromine (1.4 mL, 27 mmol) in acetic acid (10 mL) was added dropwise. After one hour the mixture was filtered, the solids washed with acetic acid (10 mL) and the filtrate concentrated. The mixture was dissolved in ethanol (40 mL) and hydrazine hydrate (4 mL) added. After 3 hours the mixture was added to ethyl acetate (100 mL) and brine (100 mL). The aqueous phase was extracted with further ethyl acetate (100 mL), and the aqueous phases discarded. The pooled ethyl acetate phases were washed with brine (100 mL), and the brine extracted with ethyl acetate (100 mL). The pooled ethyl acetate phases were dried over sodium sulfate and evaporated. The residue was diluted with 1,4-dioxane (20 mL) and treated with 33% HBr in acetic acid (4 mL) dropwise. The dark suspension was filtered, the collected solids washed with 1,4-dioxane (2×20 mL), acetone (20 mL) and air dried to give the desired compound (4.41 g, 68% yield) as a solid. ¹H NMR (400 MHz, d₆-DMSO) δ 9.51 (dd, J=2.5, 1.1 Hz, 1H), 9.15 (dd, J=5.7, 1.0 Hz, 1H), 8.16 (dd, J=5.6, 2.5 Hz, 1H). LCMS-B: Retention Time 2.80 min; m/z 159.0 [M+H]⁺ for ⁷⁹Br (free base)

### (b) 3-(Pyridazin-4-yl)benzoic acid (I15)

3-(4,4,5,5-Tetramethyl-1,3,2-dioxaborolan-2-yl)benzoic acid (0.50 g, 2.0 mmol), 4-bromopyridazine hydrobromide (I14) (0.58 g, 2.4 mmol), PdCl₂(dppf) DCM complex (83 mg, 5 mol %) and 1,4-dioxane (10 mL) were loaded into a microwave tube. A solution of potassium carbonate (0.83 g, 6.0 mmol) in water (5 mL) was added, the mixture degassed with a stream of nitrogen bubbles then heated in the microwave (120° C./30 minutes). The mixture was cooled, and the volatile solvents removed in vacuo. The aqueous residue was diluted with water to 75 mL and shaken with DCM (75 mL). The mixture was filtered through Celite, the aqueous layer separated and washed with further DCM (75 mL). The DCM extracts were discarded, the aqueous phase was diluted with water (25 mL) and treated with 5% w/v citric acid until pH 3 to pH paper. The resulting precipitate was collected by filtration, washed with water and dried under vacuum to give the desired compound (312 mg, 78% yield) as a solid. ¹H NMR (400 MHz, d₆-DMSO) δ 9.70-9.65 (m, 1H), 9.33-9.28 (m, 1H), 8.38 (s, 1H), 8.17 (d, J=7.8 Hz, 1H), 8.12-8.05 (m, 2H), 7.71 (t, J=7.8 Hz, 1H). Acyl proton not observed. LCMS-B: Retention Time 3.15 min, m/z 201.1 [M+H]⁺; m/z 199.1 [M−H]⁻

### (vii) 4-(5-Morpholino-1,3,4-oxadiazol-2-yl)benzoic acid (I17)

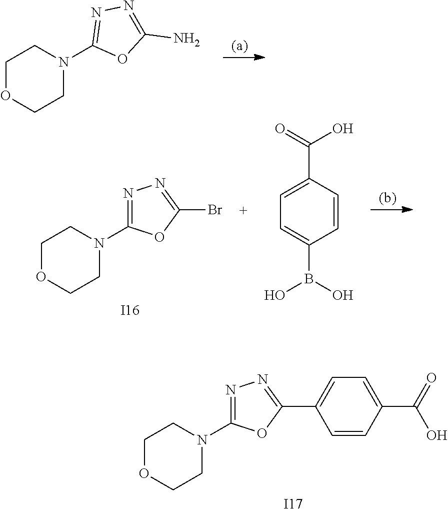

### (a) 4-(5-Bromo-1,3,4-oxadiazol-2-yl)morpholine (I16)

5-Morpholino-1,3,4-oxadiazol-2-amine (250 mg, 1.47 mmol) and copper(II) bromide (492 mg, 2.20 mmol) were stirred in acetonitrile (15 mL) under nitrogen for 5 minutes. tert-Butyl nitrite (0.349 mL, 2.94 mmol) was added and the mixture stirred at room temperature for 17 hours. The mixture was diluted with 0.5M HCl (25 mL) and ethyl acetate (25 mL). The organic phase was separated, and the aqueous phase extracted with further ethyl acetate (2×25 mL). The combined organic phases were washed with brine (30 mL), dried over sodium sulfate and evaporated. Chromatography (12 g silica cartridge, 0-60% ethyl acetate/hexanes) gave the desired compound (104 mg, 30% yield) as a solid. ¹H NMR (400 MHz, CDCl₃) δ 3.82-3.77 (m, 4H), 3.52-3.48 (m, 4H). LCMS-B: Retention Time 3.22 min; m/z 236.1 [M+H]⁺ for ⁸¹Br

### (b) 4-(5-Morpholino-1,3,4-oxadiazol-2-yl)benzoic acid (I17)

4-(5-Bromo-1,3,4-oxadiazol-2-yl)morpholine (I16) (102 mg, 0.436 mmol), 4-boronobenzoic acid (108 mg, 0.654 mmol), PdCl₂(dppf) DCM complex (18 mg, 5 mol %), and 1,4-dioxane (3 mL) were degassed with a stream of nitrogen bubbles. A 1.0M aqueous solution of cesium carbonate (1.5 mL, 1.5 mmol) was added, the mixture again degassed with a stream of nitrogen bubbles, then heated in a microwave (120° C./30 minutes). The mixture was diluted with water (20 mL) and diethyl ether (20 mL), filtered through Celite, the organic phase was discarded, and the aqueous phase was concentrated in vacuo. The residue was dissolved in water (10 mL) and the pH adjusted to 1 with 6M HCl. The precipitate was collected by filtration, the supernatant discarded, the solid resuspended in water (5 mL) and again collected by filtration. The collected solid was repeatedly suspended in absolute ethanol (20 mL) and the solvents removed in vacuo (three times) to give the desired compound (115 mg, 96% yield) as a solid of approximately 80% purity. ¹H NMR (400 MHz, d₆-DMSO) δ 8.10-8.05 (m, 2H), 8.02-7.97 (m, 2H), 3.76-3.71 (m, 4H), 3.53 (m, overlaps with solvent). LCMS-B: Retention Time 3.25 min; m/z 276.2 [M+H]⁺; m/z 274.1 [M−H]⁻

### (viii) 2-Fluoro-4-(morpholine-4-carbonyl)benzoic acid (I20)

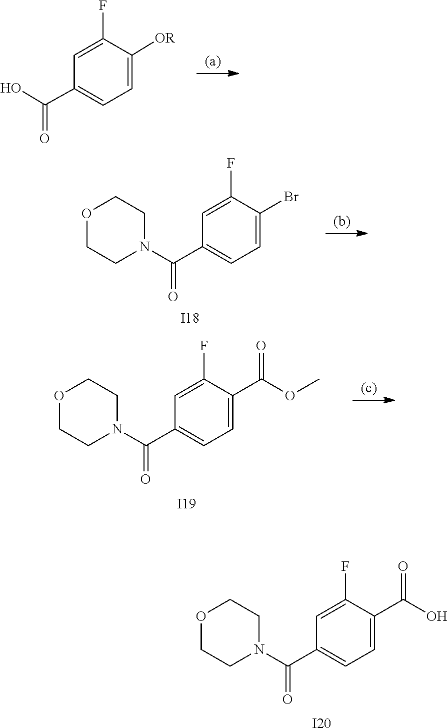

### (a) (4-Bromo-3-fluorophenyl)(morpholino)methanone (I18)

4-Bromo-3-fluorobenzoic acid (2.19 g, 10.0 mmol), DCM (100 mL), morpholine (2.59 mL, 30.0 mmol), DMAP (122 mg, 10 mol %) and EDCI.HCl (2.876 g, 15.0 mmol) were stirred at room temperature. After 17 hours the mixture was added to 2% w/v sodium hydroxide (100 mL). The separated aqueous phase was extracted with DCM (2×50 mL), the pooled DCM extracts washed with 1M HCl (75 mL), brine (50 mL), dried over sodium sulfate and evaporated. Chromatography (40 g silica cartridge, 0-60% ethyl acetate/hexanes) gave the desired compound (2.63 g, 91% yield) as a syrup. ¹H NMR (400 MHz, CDCl₃) δ 7.61 (dd, J=8.1, 6.8 Hz, 1H), 7.19 (dd, J=8.5, 1.9 Hz, 1H), 7.08 (ddd, J=8.1, 1.9, 0.7 Hz, 1H), 3.70 (br s, 6H), 3.45 (br s, 2H). LCMS-B: Retention Time 3.49 min; m/z 290.0, 288.1 [M+H]⁺

### (b) Methyl 2-fluoro-4-(morpholine-4-carbonyl)benzoate (I19)

(4-Bromo-3-fluorophenyl)(morpholino)methanone (I18) (2.62 g, 9.09 mmol), dry methanol (20 mL), PdCl₂(dppf) DCM complex (376 mg, 5 mol %) and triethylamine (2.54 mL, 18.2 mmol) were loaded into a Schlenk tube and flushed with nitrogen. The tube was flushed with carbon monoxide and the mixture brought to reflux under carbon monoxide (balloon). After 18 hours the mixture was cooled to room temperature, filtered through Celite and the Celite washed with methanol (40 mL). The combined filtrates were evaporated, chromatography (40 g silica cartridge, 20-60% ethyl acetate/hexanes) gave the desired compound (2.25 g, 93% yield) as a solid. ¹H NMR (400 MHz, CDCl₃) δ 7.99 (t, J=7.3 Hz, 1H), 7.25-7.17 (m, 2H), 3.94 (s, 3H), 3.78 (br s, 4H), 3.63 (br s, 2H), 3.40 (br s, 2H). LCMS-A: Retention Time 5.30 min; m/z 268.1 [M+H]⁺

### (c) 2-Fluoro-4-(morpholine-4-carbonyl)benzoic acid (I20)

Methyl 2-fluoro-4-(morpholine-4-carbonyl)benzoate (I19) (1.00 g, 3.74 mmol) was dissolved in THF (20 mL) and a solution of lithium hydroxide monohydrate (188 mg, 4.49 mmol) in water (10 mL) was added and the mixture was stirred vigorously at room temperature. After 2 hours, the volatile solvents were removed in vacuo and the aqueous residue cooled to 4° C. Cold 3.0M aqueous HCl (5 mL) was added, the resulting slurry diluted with water (5 mL), filtered, the collected solids washed with water (5 mL) and air dried to give the desired compound (817 mg, 86% yield) as a solid. ¹H NMR (400 MHz, d₄-MeOD) δ 8.05-7.99 (m, 1H), 7.32 (s, 1H), 7.30 (dd, J=3.7, 1.4 Hz, 1H), 3.76 (br s, 4H), 3.63 (br s, 2H), 3.42 (br s, 2H). LCMS-B: Retention Time 3.20 min; m/z 254.2 [M+H]⁺; 276.2 [M+Na]⁺

### (ix) 3-(Pyrimidin-5-yl)benzoic acid (I21)

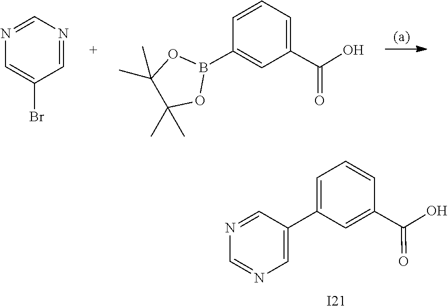 [SMILES: Brc1cncnc1 ; CC1(C)OB(c2cccc(C(=O)O)c2)OC1(C)C ; "I21" ; O=C(O)c1cccc(-c2cncnc2)c1]

### (a) 3-(Pyrimidin-5-yl)benzoic acid (I21)

3-(4,4,5,5-Tetramethyl-1,3,2-dioxaborolan-2-yl)benzoic acid (496 mg, 2.00 mmol), 5-bromopyrimidine (382 mg, 2.40 mmol), PdCl₂(dppf) DCM complex (82 mg, 5 mol %) were stirred in 1,4-dioxane (10 mL) under nitrogen and a solution of potassium carbonate (829 mg, 6.00 mmol) in water (5 mL) was added. The mixture was degassed with a stream of nitrogen bubbles and heated in the microwave (120° C./30 minutes). The volatiles were removed in vacuo and the aqueous residue diluted with water (50 mL) and DCM (50 mL). The mixture was filtered through Celite, the DCM phase discarded, and the aqueous phase extracted with further DCM (2×50 mL). The DCM extracts were again discarded, the aqueous phase was adjusted to pH 3 with 30% w/v aqueous NaHSO₄ and the precipitate collected by filtration, and dried under vacuum (40° C./3 hours over P₂O₅) to give the desired compound (137 mg, 34% yield) as a grey solid. LCMS-B: Retention Time 3.25 min; m/z 201.1 [M+H]⁺

### (x) Lithium 4-((1-acetylpiperidin-4-yl)(methyl)carbamoyl)benzoate (I26)

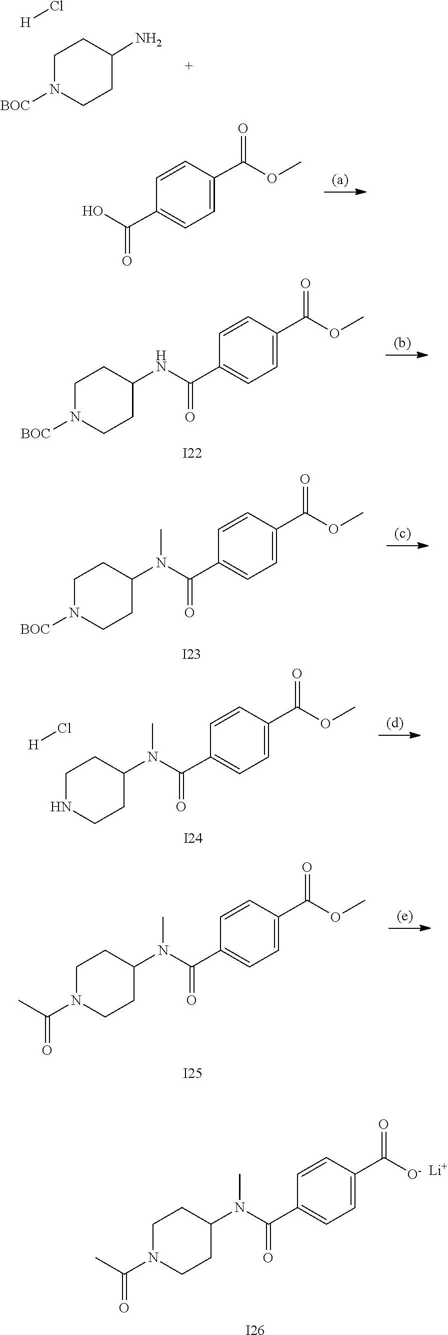

### (a) tert-Butyl 4-(4-(methoxycarbonyl)benzamido)piperidine-1-carboxylate (I22)

4-(Methoxycarbonyl)benzoic acid (1.00 g, 5.55 mmol), DCM (25 mL), DMAP (34 mg, 5 mol %), tert-butyl 4-aminopiperidine-1-carboxylate hydrochloride salt (1.45 g, 6.11 mmol) and EDCI.HCl (1.28 g, 6.66 mmol) were stirred at room temperature. After 18 hours the mixture was diluted with 10% w/v NaHSO₄ (50 mL) and DCM (50 mL). The aqueous phase was extracted with further DCM (2×50 mL), the pooled DCM extracts were washed with 1:1 saturated aqueous NaHCO₃: water (50 mL), dried over sodium sulfate and concentrated in vacuo to give the desired compound (1.82 g, 91% yield) as a solid. ¹H NMR (400 MHz, CDCl₃) δ 8.10-8.06 (m, 2H), 7.83-7.78 (m, 2H), 6.11 (d, J=7.9 Hz, 1H), 4.19-4.04 (m, 3H), 3.94 (s, 3H), 2.98-2.81 (m, 2H), 2.07-1.97 (m, 2H), 1.51-1.35 (m, 11H). LCMS-B: Retention Time 3.63 min; m/z 307.1 [M-^{t}Bu+2H]⁺, 385.2 [M+Na]⁺, 263.1 [M-Boc+2H]⁺, m/z 361.2 [M−H]⁻

### (b) tert-Butyl 4-(4-(methoxycarbonyl)-N-methylbenzamido)piperidine-1-carboxylate (I23)

tert-Butyl 4-(4-(methoxycarbonyl)benzamido)piperidine-1-carboxylate (I22) (1.00 g, 2.76 mmol) was dissolved in DMF (10 mL) and cooled to 0° C. A 60% dispersion (in mineral oil) of sodium hydride (331 mg, 8.28 mmol) and methyl iodide (0.344 mL, 5.52 mmol) were added and the mixture stirred at room temperature. After one-hour saturated ammonium chloride (10 mL) was added and the mixture added to water (200 mL). The suspension was extracted with diethyl ether (3×100 mL), the pooled ether phases washed with brine (100 mL), dried over sodium sulfate and concentrated in vacuo. Chromatography (24 g silica cartridge, 0-60% ethyl acetate/hexanes) gave the desired compound (602 mg, 58% yield) as a solid. LCMS-B: Retention Time 3.67 min; m/z 321.1 [M-^{t}Bu+2H]⁺, 277.1 [M-Boc+2H]⁺, 399.1 [M+Na]⁺

### (c) Methyl 4-(methyl(piperidin-4-yl)carbamoyl)benzoate hydrochloride salt (I24)

tert-Butyl 4-(4-(methoxycarbonyl)-N-methylbenzamido)piperidine-1-carboxylate (I23) (600 mg, 1.59 mmol), 1,4-dioxane (6 mL) and 4.0M HCl in 1,4-dioxane (6 mL) were stirred at room temperature. After 5 hours the resulting precipitate was filtered, washed with further 1,4-dioxane (2×5 mL) and air dried to give the desired compound (444 mg, 89% yield) as a solid. LCMS-B: Retention Time 3.09 min; m/z 277.1 [M+H]⁺ (free base)

### (d) Methyl 4-((I-acetylpiperidin-4-yl)(methyl)carbamoyl)benzoate (I25)

Methyl 4-(methyl(piperidin-4-yl)carbamoyl)benzoate hydrochloride salt (I24) (200 mg, 0.639 mmol), DCM (5 mL), triethylamine (0.267 mL, 1.92 mmol), DMAP (8 mg, 10 mol %) and acetyl chloride (0.068 mL, 0.96 mmol) were stirred at room temperature. After 17 hours the mixture was diluted with water (5 mL) and DCM (5 mL). The mixture was passed through a phase separation cartridge and the DCM phase concentrated in vacuo. Chromatography (12 g silica cartridge, 0-100% ethyl acetate/hexanes) gave the desired compound (176 mg, 87% yield) as a solid. LCMS-B: Retention Time 3.27 min; m/z 319.1 [M+H]⁺

### (e) Lithium 4-((I-acetylpiperidin-4-yl)(methyl)carbamoyl)benzoate (I26)

Methyl 4-((1-acetylpiperidin-4-yl)(methyl)carbamoyl)benzoate (I25) (170 mg, 0.53 mmol) was dissolved in THF (1 mL) and methanol (0.5 mL). A solution of lithium hydroxide monohydrate (25 mg, 0.59 mmol) in water (0.5 mL) was added and the mixture stirred at room temperature. After 3 hours the mixture was concentrated in vacuo, the residue was dried under vacuum over P₂O₅ to give the desired compound (153 mg, 92% yield) as a solid. LCMS-B: 3.13 min; m/z 305.1 [M-Li+2H]⁺, m/z 303.1 [M-Li]⁻

### (xi) Lithium 4-((1-(methoxycarbonyl)piperidin-4-yl)(methyl)carbamoyl)benzoate (I28)

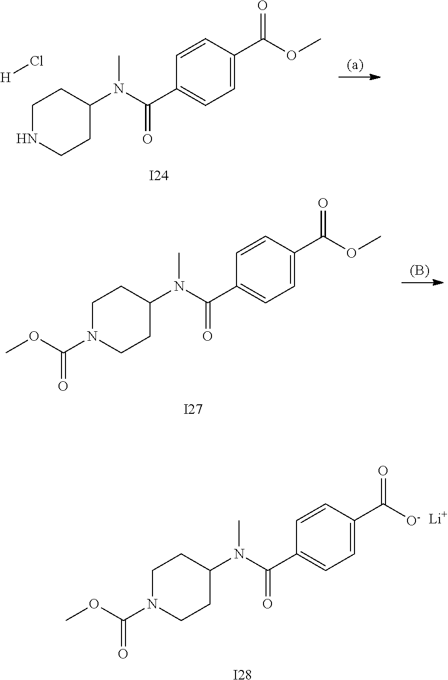

### (a) Methyl 4-(4-(methoxycarbonyl)-N-methylbenzamido)piperidine-1-carboxylate (I27)

Methyl 4-(methyl(piperidin-4-yl)carbamoyl)benzoate hydrochloride salt (I24) (200 mg, 0.639 mmol), DCM (5 mL), triethylamine (0.267 mL, 1.92 mmol), DMAP (8 mg, 10 mol %) and methyl chloroformate (0.074 mL, 0.96 mmol) were stirred at room temperature. After 17 hours the mixture was diluted with water (5 mL) and DCM (5 mL). The mixture was passed through a phase separation cartridge and the DCM phase concentrated in vacuo. Chromatography (12 g silica cartridge, 0-100% ethyl acetate/hexanes) gave the desired compound (135 mg, 63% yield) as a solid. LCMS-B: Retention Time 3.40 min; m/z 335.1 [M+H]⁺

### (b) Lithium 4-((1-(methoxycarbonyl)piperidin-4-yl)(methyl)carbamoyl)benzoate (I28)

Methyl 4-(4-(methoxycarbonyl)-N-methylbenzamido)piperidine-1-carboxylate (I27) was dissolved in THF (1 mL) and methanol (0.5 mL). A solution of lithium hydroxide monohydrate (18 mg, 0.43 mmol) in water (0.5 mL) was added and the mixture stirred at room temperature. After 3 hours the mixture was concentrated in vacuo, the residue was dried under vacuum over P₂O₅ to give the desired compound (127 mg, quant yield) as a solid. LCMS-B: Retention Time 3.26 min; m/z 321.3 [M-Li+2H]⁺; m/z 319.1 [M-Li]⁻

### (xii) 2-((Tetrahydro-2H-pyran-4-yl)oxy)isonicotinic acid (I29)

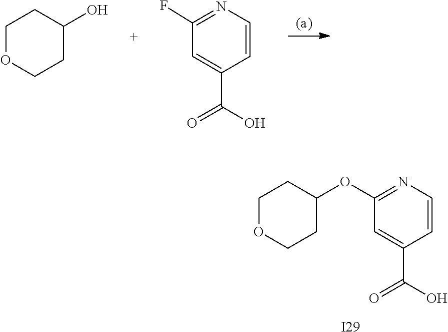 [SMILES: OC1CCOCC1 ; O=C(O)c1ccnc(F)c1 ; "I29" ; O=C(O)c1ccnc(OC2CCOCC2)c1]

### (a) 2-((Tetrahydro-2H-pyran-4-yl)oxy)isonicotinic acid (I29)

A solution of tetrahydro-2H-pyran-4-ol (0.462 g, 4.52 mmol, 2 equiv) in anhydrous DMF (5 mL) was added to a stirring suspension of sodium hydride (60% dispersion in oil, 0.362 g, 9.04 mmol, 4 equiv) in anhydrous DMF (5 mL) under an atmosphere of nitrogen. The mixture was stirred at room temperature for 10 minutes before a solution of 2-fluoroisonicotinic acid (0.319 g, 2.26 mmol, 1 equiv) in DMF (5 mL) was added. The mixture was stirred for a further 16 hours at room temperature. H₂O (˜20 mL) was carefully added and the pH of the aqueous mixture was adjusted to ˜2 with aqueous HCl (˜2M). The aqueous was extracted with EtOAc (3×30 mL), the organics were combined, washed with brine, dried (Na₂SO₄) and the solvent removed in vacuo. The resultant residue was purified by column chromatography (Biotage Isolera, 40 g SiO₂ cartridge, 0-100% EtOAc in petroleum benzine 40-60° C.) to give the desired compound (383 mg, 76% yield) as a solid. ¹H NMR (400 MHz, d₆-DMSO) δ 8.31 (dd, J=5.2, 0.8 Hz, 1H), 7.36 (dd, J=5.2, 1.4 Hz, 1H), 7.15 (dd, J=1.3, 0.7 Hz, 1H), 5.21 (tt, J=8.7, 4.1 Hz, 1H), 3.86 (dt, J=11.3, 4.3 Hz, 2H), 3.58-3.45 (m, 2H), 2.06-1.93 (m, 2H), 1.74-1.55 (m, 2H), OH not observed. LCMS-B: Retention Time 3.36 min, m/z 224 [M+H]⁺.

### (xiii) 2-((1-(Methoxycarbonyl)piperidin-4-yl)oxy)isonicotinic acid (I32)

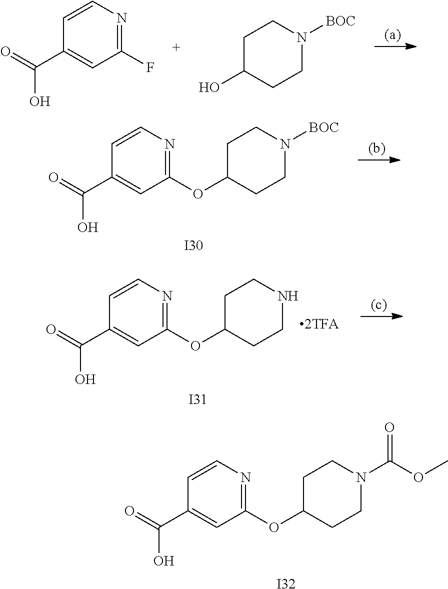

### (a) 2-((1-(tert-Butoxycarbonyl)piperidin-4-yl)oxy)isonicotinic acid (I30)

A solution of tert-butyl 4-hydroxypiperidine-1-carboxylate (0.500 g, 2.48 mmol) in anhydrous DMF (5 mL) was added to a stirring suspension of sodium hydride (60% dispersion in mineral oil, 0.238 g, 5.95 mmol) in anhydrous DMF (5 mL) under an atmosphere of nitrogen. The mixture was stirred at room temperature for 10 minutes before a solution of 2-fluoroisonicotinic acid (0.319 g, 2.26 mmol) in DMF (5 mL) was added. The mixture was stirred for a further 16 hours at room temperature and 4 hours at 60° C. After returning to room temperature, water (˜20 mL) was carefully added and the pH of the aqueous mixture was adjusted to ˜2 with aqueous HCl (˜2M). The aqueous was extracted with EtOAc (3×30 mL), the organics were combined, washed with brine, dried (MgSO₄) and the solvent removed in vacuo. The resultant residue was purified by column chromatography (Biotage Isolera, 40 g SiO₂ cartridge, 0-100% EtOAc in petroleum benzine 40-60° C.) to give a solid. Analysis by ¹H NMR showed an approximate 1:1 mixture of the desired product and the isonicotinic acid starting material. This material was reacted with another equivalent of the Boc-protected hydroxypiperidine anion: A solution of tert-butyl 4-hydroxypiperidine-1-carboxylate (0.500 g, 2.48 mmol) in anhydrous DMF (5 mL) was added to a stirring suspension of sodium hydride (60% dispersion in mineral oil, 0.238 g, 5.95 mmol) in anhydrous DMF (5 mL) under an atmosphere of nitrogen. The mixture was stirred at room temperature for 10 minutes before a solution of the 1:1 product/starting material mixture in DMF (5 mL) was added and stirring was continued for 16 hours. Water (˜20 mL) was carefully added and the pH of the aqueous mixture was adjusted to ˜2 with aqueous HCl (˜2M). The aqueous was extracted with DCM (3×30 mL), the organics were combined, dried (MgSO₄) and the solvent removed in vacuo. The resultant residue was purified by column chromatography (Biotage Isolera, 40 g SiO₂ cartridge, 0-100% EtOAc in petroleum benzine 40-60° C.) to give the desired compound (0.419 g, 58% yield) as a solid. ¹H NMR (400 MHz, d₆-DMSO) δ 8.34-8.27 (m, 1H), 7.39-7.34 (m, 1H), 7.17-7.13 (m, 1H), 5.27-5.15 (m, 1H), 3.74-3.62 (m, 2H), 3.23-3.11 (m, 2H), 2.00-1.88 (m, 2H), 1.63-1.49 (m, 2H), 1.40 (s, 9H). LCMS-B: Retention Time 3.65 min; m/z 321.2 [M−H]⁻

### (b) 2-(Piperidin-4-yloxy)isonicotinic acid bis(2,2,2-trifluoroacetic acid) salt (I31)

To a solution of 2-((1-(tert-butoxycarbonyl)piperidin-4-yl)oxy)isonicotinic acid I30 (141 mg, 0.439 mmol, 1 equiv) in DCM (10 mL) was added TFA (0.5 mL, 6.5 mmol, 15 equiv). The reaction was stirred at room temperature for 16 hours and then dried in vacuo to give an oil. The residue was taken up in diethyl ether:methanol (10 mL, 1:1) and dried in vacuo to give the desired compound (196 mg, quantitative yield) as a solid. LCMS-B: Retention Time 1.74 min, m/z 223 [M+H]⁺ (free base)

### (c) 2-((1-(Methoxycarbonyl)piperidin-4-yl)oxy)isonicotinic acid (I32)

Methylchloroformate (68 μL, 0.87 mmol, 2 equiv) was added drop-wise to a mixture of 2-(piperidin-4-yloxy)isonicotinic acid-bis(2,2,2-trifluoroacetic acid) salt I31 (196 mg, 0.437 mmol, 1 equiv) and sodium hydroxide (73 mg, 1.8 mmol) in water (10 mL). The reaction was stirred at ambient temperature overnight. The aqueous phase was separated and adjusted to pH 1 with a 1M aqueous solution of HCl. The aqueous phase was extracted with EtOAc (3×20 mL), and the combined organic layers were dried over Na₂SO₄, filtered, and the filtrate concentrated in vacuo. The resultant oil was purified by column chromatography (12 g SiO₂ cartridge, 0-100% EtOAc in petroleum benzine 40-60° C.) to give the desired compound (103 mg, 84% yield) as a glassy solid. ¹H NMR (400 MHz, CDCl₃) δ 8.29 (dd, J=5.3, 0.8 Hz, 1H), 7.45 (dd, J=5.3, 1.4 Hz, 1H), 7.36 (dd, J=1.4, 0.8 Hz, 1H), 6.98 (br s, 1H), 5.27 (tt, J=7.4, 3.6 Hz, 1H), 3.86-3.75 (m, 2H), 3.73 (s, 3H), 3.49-3.34 (m, 2H), 2.06-1.95 (m, 2H), 1.86-1.70 (m, 2H)

### (xiv) Lithium 5-(morpholine-4-carbonyl)picolinate (I34)

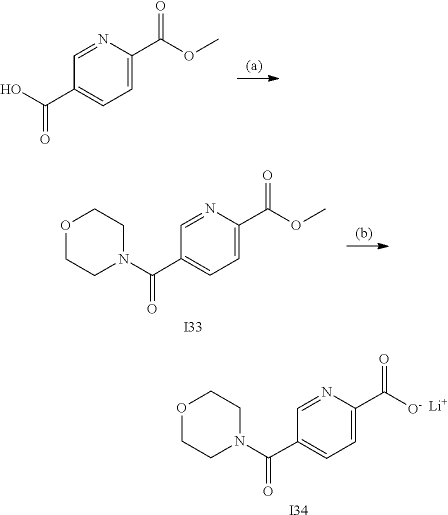 [SMILES: COC(=O)c1ccc(C(=O)O)cn1 ; "I33" ; "I34" ; COC(=O)c1ccc(C(=O)N2CCOCC2)cn1 ; O=C([O-])c1ccc(C(=O)N2CCOCC2)cn1 ; [Li+]]

### (a) Methyl 5-(morpholine-4-carbonyl)picolinate (I33)

6-(Methoxycarbonyl)nicotinic acid (1.00 g, 5.52 mmol), DCM (30 mL), DMAP (67 mg, 10 mol %), morpholine (1.43 mL, 16.6 mmol) and EDCI.HCl (1.59 g, 8.28 mmol) were stirred together at room temperature. After 18 hours the mixture was diluted with water, the organic phase separated, and the aqueous phase extracted with DCM (2×30 mL). The pooled organic extracts were washed with brine and concentrated in vacuo. Chromatography (40 g silica cartridge, 0-100% ethyl acetate/hexanes) gave the desired compound (1.108 g, 80% yield) as an oil that became solid on standing. LCMS-B: Retention Time 3.12 min; m/z 251.2 [M+H]⁺

### (b) Lithium 5-(morpholine-4-carbonyl)picolinate (I34)

Methyl 5-(morpholine-4-carbonyl)picolinate (I33) (1.11 g, 4.43 mmol) was dissolved in THF (10 mL) and a solution of lithium hydroxide monohydrate (204 mg, 4.87 mmol) in water (5 mL) was added. After 2 hours the mixture was concentrated in vacuo, the residue was dried by evaporation with absolute ethanol (3×100 mL), washed with diethyl ether (100 mL) and dried in vacuo to give the desired compound (0.995 g, 89% yield) as a solid. LCMS-A: Retention Time 4.01 min; m/z 237.6 [M-Li+2H]⁺; m/z 235.6 [M-Li]⁻

### (xv) Lithium 4-(((R)-2-((S)-2-(tert-butoxycarbonyl)-1,2,3,4-tetrahydroisoquinolin-3-yl)-2-hydroxyethyl)carbamoyl)benzoate (I36)

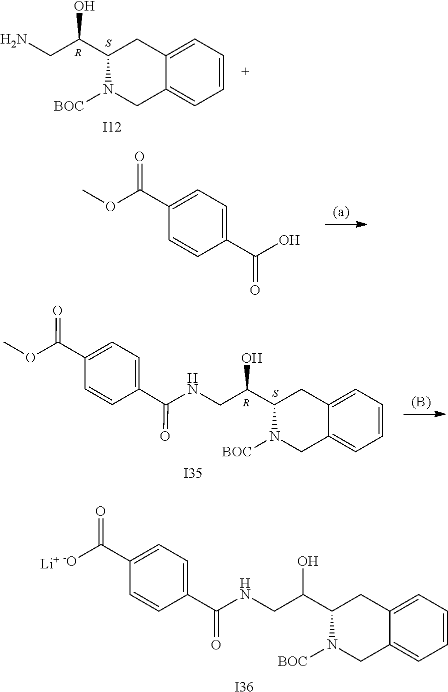

### (a) tert-Butyl (S)-3-((R)-1-hydroxy-2-(4-(methoxycarbonyl)benzamido)ethyl)-3,4-dihydroisoquinoline-2(1H)-carboxylate (I35)

tert-Butyl (S)-3-((R)-2-amino-1-hydroxyethyl)-3,4-dihydroisoquinoline-2(1H)-carboxylate (I12) (400 mg, 1.37 mmol), MeCN (10 mL), triethylamine (0.381 mL, 2.74 mmol), 4-(methoxycarbonyl)benzoic acid (246 mg, 1.37 mmol) and HATU (780 mg, 2.05 mmol) were stirred together at room temperature. After 18 hours the mixture was concentrated in vacuo, chromatography (12 g silica cartridge, 0-60% ethyl acetate/hexanes) gave the desired compound (309 mg, 50% yield) as an oil. ¹H NMR (400 MHz, d₄-MeOD) δ 8.11-8.03 (m, 2H), 7.94-7.84 (m, 2H), 7.22-7.10 (m, 4H), 4.48-4.23 (m, 2H), 3.92 (s, 3H), 3.85-3.70 (m, 1H), 3.68-3.57 (m, 1H), 3.22 (dd, J=16.1, 2.8 Hz, 1H), 2.95 (dd, J=15.9, 5.3 Hz, 1H), 1.56-1.45 (m, 9H). LCMS-B: Retention Time 3.80 min; m/z 355.3 [M-Boc+2H]⁺; m/z 453.2 [M−H]⁻

### (b) Lithium 4-(((R)-2-((S)-2-(tert-butoxycarbonyl)-1,2,3,4-tetrahydroisoquinolin-3-yl)-2-hydroxyethyl)carbamoyl)benzoate (I36)

tert-Butyl (S)-3-((R)-1-hydroxy-2-(4-(methoxycarbonyl)benzamido)ethyl)-3,4-dihydroisoquinoline-2(1H)-carboxylate (I35) (307 mg, 0.675 mmol) was dissolved in THF (2 mL) and a solution of lithium hydroxide monohydrate (31 mg, 0.74 mmol) in water (1 mL) was added. After 18 hours the mixture was concentrated in vacuo, the residue was dried by evaporation with absolute ethanol (3×20 mL) to give the crude desired compound (308 mg) as a solid. LCMS-B: Retention Time 3.58 min; m/z 439.2 [M-Li+2H]⁻

### (xvi) 4-(3-Oxa-8-azabicyclo[3.2.1]octane-8-carbonyl)benzoic acid (I38)

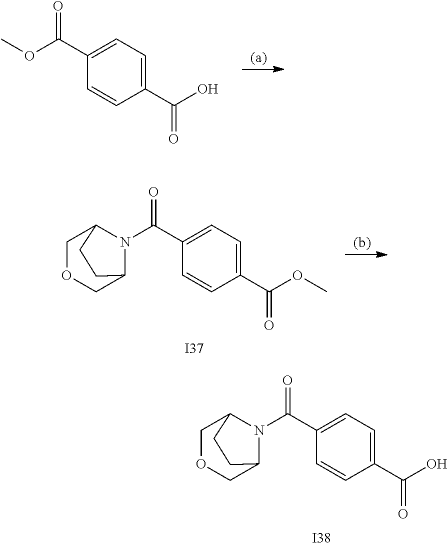 [SMILES: COC(=O)c1ccc(C(=O)O)cc1 ; "I37" ; "I38" ; COC(=O)c1ccc(C(=O)N2C3CCC2COC3)cc1 ; O=C(O)c1ccc(C(=O)N2C3CCC2COC3)cc1]

### (a) Methyl 4-(3-oxa-8-azabicyclo[3.2.1]octane-8-carbonyl)benzoate (I37)

To a solution of 4-(methoxycarbonyl)benzoic acid (0.66 g, 3.7 mmol) in DCM (20 mL) was added, 3-oxa-8-azabicyclo[3.2.1]octane hydrochloride (0.50 g, 3.4 mmol), DIPEA (0.87 g, 6.7 mmol), HOBt (45 mg, 0.3 mmol) and EDCI (0.77 g, 4.0 mmol). The resulting mixture was stirred at room temperature overnight. The mixture was partitioned against saturated aqueous NaHCO₃ (20 mL×2) and the aqueous layer extracted with DCM (5 mL×2). The combined organic layers were washed with brine (20 mL×2), dried (Na₂SO₄) and concentrated. The crude residue obtained was purified by column chromatography (1% methanol/DCM) to give the desired compound (0.79 g, 85% yield) as a solid. LCMS-C: Retention Time 0.61 min; m/z 276.1 [M+H]⁺

### (b) 4-(3-Oxa-8-azabicyclo[3.2.1]octane-8-carbonyl)benzoic acid (I38)

To a solution of methyl 4-(3-oxa-8-azabicyclo[3.2.1]octane-8-carbonyl)benzoate I37 (0.77 g, 2.8 mmol) in a mixture of THF (20 mL), methanol (2 mL) and water (2 mL) was added LiOH—H₂O (0.59 g, 14 mmol). The resulting mixture was stirred at room temperature overnight, then the solvent was removed, and the residue obtained diluted with water (20 mL). The pH of the aqueous solution was adjusted to 6 by addition of 2 M HCl. The aqueous layer was extracted with DCM (20 mL×3) and the combined organic layers washed with brine (20 mL×2), dried (Na₂SO₄) and concentrated to give the desired compound (0.46 g, 63% yield) as a solid. LCMS-C: Retention Time 0.83 min; m/z 262.1 [M+H]⁺

### (xvii) 4-(3-Oxa-8-azabicyclo[3.2.1]octane-8-carbonyl)-2-ethoxybenzoic acid (I42)

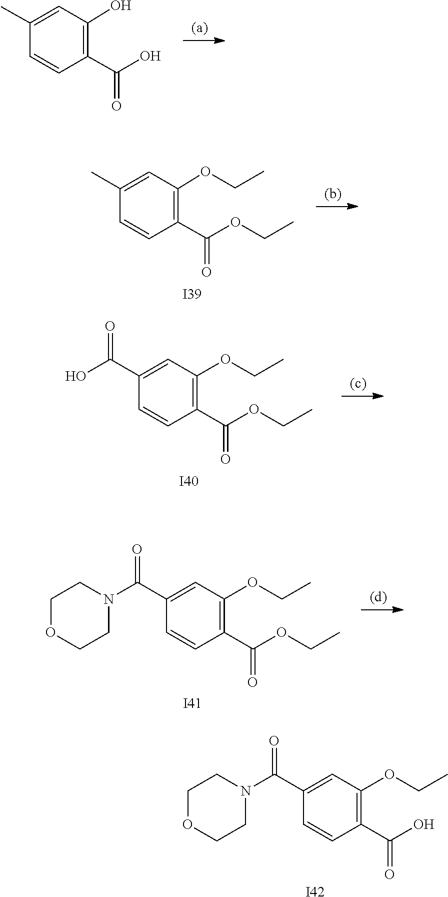

### (a) Ethyl-2-ethoxy-4-methylbenzoate (I39)

To a mixture of 2-hydroxy-4-methylbenzoic acid (8.2 g, 54 mmol) and K₂CO₃ (22.4 g, 162 mmol) in DMSO (70 mL) at 40° C. was added ethyl iodide (12.6 g, 80.8 mmol) drop-wise over a period of 30 minutes. The reaction was stirred for 2 hours then further ethyl iodide (12.6 g, 80.8 mmol) was added over 30 minutes. The resulting mixture was stirred for another 8 hours at 40° C., then diluted with DCM (150 mL) and filtered. The filtrate was washed with water (200 mL×10) and brine (200 mL×2), dried (Na₂SO₄) and concentrated to give the desired compound (10.1 g, 90% yield) as a liquid. LCMS-C: Retention Time 2.70 min; m/z 209.1 [M+H]⁺

### (b) 3-Ethoxy-4-(ethoxycarbonyl)-benzoic acid (I40)

To a solution of ethyl 2-ethoxy-4-methylbenzoate I39 (10.0 g, 48.1 mmol) in a mixture of pyridine (25 mL) and water (75 mL) was added KMnO₄ (22.8 g, 144 mmol). The resulting mixture was heated at 50° C. for 48 hours, then cooled and allowed to stir at room temperature 24 hours. The mixture was filtered, and the filter cake washed with hot water. The combined aqueous filtrates were washed with EtOAc (75 mL×3) and acidified with 2M HCl solution. The mixture was extracted with DCM (150 mL×3), the combined DCM layers were washed with brine, dried (Na₂SO₄) and concentrated to give the desired compound (5.0 g, 44% yield) as a solid. LCMS-C: Retention Time 0.25 min; m/z 239.0 [M+H]⁺

### (c) Ethyl-4-(3-oxa-8-azabicyclo[3.2.1]octane-8-carbonyl)-2-ethoxybenzoate (I41)

To a solution of 3-ethoxy-4-(ethoxycarbonyl)benzoic acid I40 (2.5 g, 10.4 mmol) in DCM (20 mL) was added 3-oxa-8-azabicyclo[3.2.1]octane hydrochloride (1.4 g, 9.5 mmol), HOBt (135.1 mg, 1.0 mmol), DIPEA (2.5 g, 19.0 mmol) and EDCI (2.2 g, 11.4 mmol). The resulting mixture was stirred at room temperature overnight. The mixture was partitioned against saturated aqueous NaHCO₃, and extracted with DCM (20 mL×2). The combined organic layers were washed with brine, dried (Na₂SO₄) and concentrated. The residue was purified by column chromatography (1% methanol/dichloromethane) to give the desired compound (2.5 g, 80% yield) as an oil. LCMS-C: Retention Time 2.40 min; m/z 334.1 [M+H]⁺

### (d) 4-(3-Oxa-8-azabicyclo[3.2.1]octane-8-carbonyl)-2-ethoxybenzoic acid (I42)

To a solution of ethyl-4-(3-oxa-8-azabicyclo-[3.2.1]-octane-8-carbonyl)-2-ethoxybenzoate I41 (2.4 g, 7.2 mmol) in a mixture of THF (20 mL), methanol (2 mL) and water (2 mL) was added LiOH—H₂O (1.5 g, 36 mmol). The resulting mixture was stirred at room temperature for 24 hours. The solvent was removed, and the residue obtained diluted with water (20 mL), the pH of the aqueous mixture was adjusted to 6 by addition of 2M HCl. The mixture was extracted with DCM (20 mL×3) and the combined organic layers washed with brine (10 mL×2), dried (Na₂SO₄) and concentrated to give the desired compound (1.7 g, 79% yield) as an oil. ¹H NMR (400 MHz, d₄-MeOD) δ 7.83 (d, J=7.8 Hz, 1H), 7.19 (d, J=1.0 Hz, 1H), 7.10 (dd, J=7.8, 1.3 Hz, 1H), 4.65 (br s, 1H), 4.20 (q, J=7.0 Hz, 2H), 3.97 (br s, 1H), 3.82 (d, J=10.8 Hz, 1H), 3.72 (d, J=11.0 Hz, 2H), 3.59 (d, J=10.9 Hz, 1H), 2.13-1.94 (m, 4H), 1.45 (t, J=7.0 Hz, 3H). LCMS-C: Retention Time 1.20 min; m/z 306.1 [M+H]⁺.

Alternate Synthesis Method

### (a) Ethyl-2-ethoxy-4-methylbenzoate (I39)

To a mixture of 2-hydroxy-4-methylbenzoic acid (20 g, 131.5 mmol) and K₂CO₃ (54.5 g, 394.5 mmol) in DMSO (50 mL) was added ethyl iodide (21.5 g, 197.2 mmol) drop-wise over a period of 30 minutes. The reaction was stirred for 2 hours then further ethyl iodide (21.5 g, 197.3 mmol) was added over 30 minutes. The resulting mixture was stirred for another 8 hours at 40° C., then diluted with DCM (150 mL) and filtered. The filtrate was washed with water (150 mL×15) and brine (100 mL×2), dried (Na₂SO₄) and concentrated to give the desired compound as an oil (27 g, 98%). LCMS-C: Retention Time 2.70 min; m/z 209.1 [M+H]⁺

### (b) 3-Ethoxy-4-(ethoxycarbonyl)-benzoic acid (I40)

To a solution of ethyl 2-ethoxy-4-methylbenzoate I39 (10.0 g, 48.1 mmol) in a mixture of pyridine (25 mL) and water (75 mL) was added KMnO₄ (22.8 g, 144 mmol). The resulting mixture was heated at 50° C. for 48 hours, then cooled and allowed to stir at room temperature for 24 hours. The mixture was filtered and the filter cake washed with hot water, the combined aqueous filtrates were washed with EtOAc (75 mL×3) and acidified with 2M HCl solution. The aqueous was extracted with DCM (150 mL×3), the combined organic layers were washed with brine, dried (Na₂SO₄) and concentrated to give the desired compound (5.0 g, 44% yield) as a solid. LCMS-C: Retention Time 0.25 min; m/z 239.0 [M+H]⁺

### (c) Ethyl-4-(3-oxa-8-azabicyclo[3.2.1]octane-8-carbonyl)-2-ethoxybenzoate (I41)

To a solution of 3-ethoxy-4-(ethoxycarbonyl)benzoic acid I40 (10.0 g, 42 mmol) in DCM (20 mL) was added 3-oxa-8-azabicyclo[3.2.1]octane hydrochloride (5.7 g, 38.2 mmol), HOBt (0.52 g, 3.8 mmol), DIPEA (9.8 g, 76.2 mmol) and EDCI (8.8 g, 45.7 mmol). The resulting mixture was stirred at room temperature overnight. The above reaction was repeated from 8.5 g of 3-ethoxy-4-(ethoxycarbonyl)benzoic acid and the two batches combined and worked up together. The reaction was partitioned against saturated aqueous NaHCO₃, and extracted with DCM (200 mL×2). The combined organic layers were washed with brine, dried (Na₂SO₄) and concentrated. The residue was purified by column chromatography (50% ethyl acetate/petroleum ether) to give the desired compound (20.5 g, 87%) as an oil. LCMS-C: Retention Time 2.40 min; m/z 334.1 [M+H]⁺

### (d) 4-(3-Oxa-8-azabicyclo[3.2.1]octane-8-carbonyl)-2-ethoxybenzoic acid (I42)

To a solution of ethyl-4-(3-oxa-8-azabicyclo-[3.2.1]-octane-8-carbonyl)-2-ethoxybenzoate I41 (20.4 g, 61.2 mmol) in a mixture of THF (150 mL), methanol (15 mL) and water (15 mL) was added LiOH—H₂O (12.9 g, 306.1 mmol). The resulting mixture was stirred at room temperature for 48 hours. The solvent was removed, and the residue obtained diluted with water (50 mL), the pH of the aqueous mixture was adjusted to 4 by addition of 2 M HCl. The mixture was extracted with DCM (150 mL×3) and the combined organic layers washed with brine (200 mL×2), dried (Na₂SO₄) and concentrated to give the desired compound (16.8 g, 90%) as a solid. ¹H NMR (400 MHz, d₄-MeOD) δ 7.83 (d, J=7.8 Hz, 1H), 7.19 (d, J=1.0 Hz, 1H), 7.10 (dd, J=7.8, 1.3 Hz, 1H), 4.65 (br s, 1H), 4.20 (q, J=7.0 Hz, 2H), 3.97 (br s, 1H), 3.82 (d, J=10.8 Hz, 1H), 3.72 (d, J=11.0 Hz, 2H), 3.59 (d, J=10.9 Hz, 1H), 2.13-1.94 (m, 4H), 1.45 (t, J=7.0 Hz, 3H). LCMS-C: Retention Time 1.20 min; m/z 306.1 [M+H]⁺.

### (xviii) 6-((1-Acetylpiperidin-4-yl)amino)pyrimidine-4-carboxylic acid (I47)

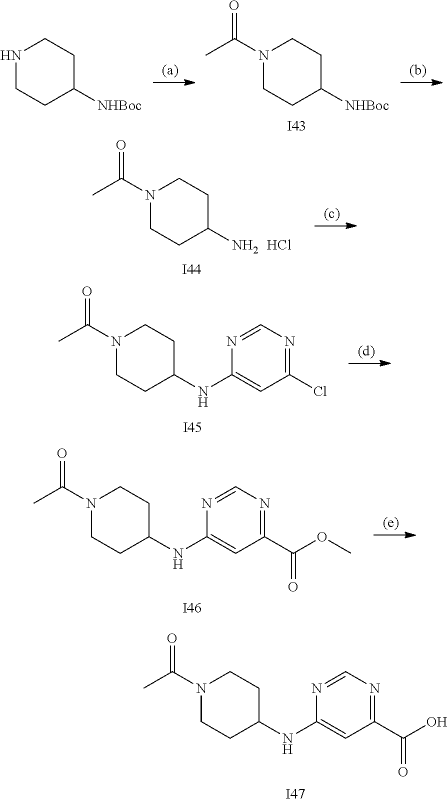

### (a) tert-Butyl (1-acetylpiperidin-4-yl)carbamate (I43)

To a solution of tert-butyl piperidin-4-ylcarbamate (5.0 g, 25 mmol) in DCM (80 mL) at 0° C. were added Et₃N (3.8 g, 38 mmol) and Ac₂O (2.6 g, 25 mmol). The resulting mixture was stirred at 0° C. for 2 hours. The reaction was quenched with water (30 mL), separated and the organic layer was washed with saturated NaHCO₃ (30 mL), dried over Na₂SO₄ and concentrated to give the desired compound (5.6 g, 93% yield) as a solid. LCMS-C: Retention Time 1.88 min; m/z 265.1[M+Na]⁺

### (b) 1-(4-Aminopiperidin-1-yl)ethanone hydrochloride (I44)

To a solution of tert-butyl (1-acetylpiperidin-4-yl)carbamate 143 (2.5 g, 10 mmol) in MeOH (10 mL) was added HCl/EtOAc (2 M, 10 mL). The mixture was stirred at room temperature overnight. The solvent was removed to give the desired compound (1.6 g, 89% yield) as a solid. LCMS-C: Retention Time 0.25 min; m/z 143.1[M+H]⁺ (free base)

### (c) 1-(4-((6-Chloropyrimidin-4-yl)amino)piperidin-1-yl)ethanone (I45)

To a solution of 1-(4-aminopiperidin-1-yl)ethanone hydrochloride I44 (1.0 g, 5.6 mmol) in i-PrOH (10 mL) were added DIPEA (2.17 g, 16.8 mmol) and 4,6-dichloropyrimidine (0.83 g, 5.6 mmol). The resulting mixture was stirred at 100° C. in a sealed tube overnight. The solvent was removed under reduced pressure, the residue was diluted with water (50 mL) and the pH adjusted to 12 by addition of 1 M NaOH. The aqueous layer was extracted with DCM (4×50 mL), the combined organic layers were washed with brine (100 mL), dried over Na₂SO₄ and concentrated. The residue was purified by column chromatography (100% EtOAc) to give the desired compound (1.3 g, 92% yield) as an oil. ¹H NMR (400 MHz, d₄-MeOD) δ 8.24 (s, 1H), 6.50 (s, 1H), 4.44-4.41 (m, 1H), 4.17 (m, 1H), 3.94-3.90 (m, 1H), 3.28-3.20 (m, 1H), 2.91-2.84 (m, 1H), 2.12-2.05 (m, 5H), 1.52-1.40 (m, 2H). LCMS-C: Retention Time 0.73 min; m/z 255.1[M+H]⁺

### (d) Methyl 6-((1-acetylpiperidin-4-yl)amino)pyrimidine-4-carboxylate (I46)

To a solution of 1-(4-((6-chloropyrimidin-4-yl)amino)piperidin-1-yl)ethanone I45 (1.7 g, 6.7 mmol) in MeOH (25 mL) were added Et₃N (2.0 g, 20 mmol) and PdCl₂(dppf) (0.24 g, 0.33 mmol). The reaction was heated at reflux overnight under an atmosphere of carbon monoxide (balloon). The solvent was removed under reduced pressure, the residue was diluted with water (50 mL) and the aqueous layer extracted with DCM (3×50 mL). The combined organic layers were washed with brine (100 mL), dried over Na₂SO₄ and concentrated. The residue was purified by column chromatography (0-5% MeOH/DCM) to give the desired compound (1.2 g, 65% yield) as a red solid. LCMS-C: Retention Time 0.49 min; m/z 279.1[M+H]⁺

### (e) 6-((1-Acetylpiperidin-4-yl)amino)pyrimidine-4-carboxylic acid (I47)

To a solution of methyl 6-((1-acetylpiperidin-4-yl)amino)pyrimidine-4-carboxylate I46 (100 mg, 0.34 mmol) in MeOH (3 mL) was added a solution of NaOH (27 mg, 0.68 mmol) in water (1 mL). The resulting mixture was stirred at room temperature overnight. The pH of the solution was adjusted to pH 6 by addition of 3 M HCl and concentrated. The residue was lyophilized to give the crude desired compound (110 mg) as a solid. LCMS-C: Retention Time 0.28 min; m/z 265.1 [M+H]⁺

### (xix) 3-((1-(Methoxycarbonyl)piperidin-4-yl)oxy)benzoic acid (I48)

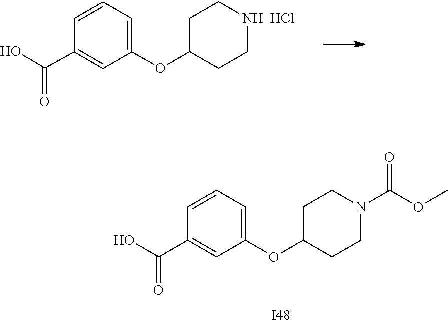 [SMILES: O=C(O)c1cccc(OC2CCNCC2)c1 ; Cl ; "I48" ; COC(=O)N1CCC(Oc2cccc(C(=O)O)c2)CC1]

Methyl chloroformate (240 μL, 3.10 mmol, 2 equiv) was added drop-wise to a mixture of 3-(piperidin-4-yloxy)benzoic acid hydrochloride (400 mg, 1.55 mmol, 1 equiv) and sodium hydroxide (261 mg, 6.52 mmol, 4.2 equiv) in water (10 mL). The reaction was stirred at ambient temperature overnight, acidified to pH=3 with a 0.5 M aqueous solution of citric acid and extracted with EtOAc (3×20 mL). The combined organic layers were dried over Na₂SO₄, filtered, and the filtrate concentrated in vacuo. The crude product was purified by column chromatography (12 g SiO₂ cartridge, 0-60% EtOAc in petroleum benzine 40-60° C.) to give the title compound (201 mg, 46% yield) as an oil. ¹H NMR (400 MHz, CDCl₃) δ 9.47 (s, 1H), 7.72-7.68 (m, 1H), 7.61 (dd, J=2.6, 1.5 Hz, 1H), 7.36 (t, J=7.9 Hz, 1H), 7.16-7.10 (m, 1H), 4.56 (tt, J=6.9, 3.4 Hz, 1H), 3.77-3.65 (m, 5H), 3.54-3.38 (m, 2H), 1.99-1.87 (m, 2H), 1.84-1.71 (m, 2H). LCMS-B: Retention Time 3.09 min, m/z 280.1 [M+H]⁺, 278.1 [M−H]⁻.

### (xx) 2-(Piperidin-4-yloxy)isonicotinic acid dihydrochloride (I49)

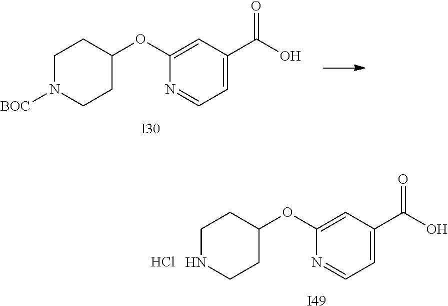 [SMILES: "I30" ; CC(C)(C)OC(=O)N1CCC(Oc2cc(C(=O)O)ccn2)CC1 ; "I49" ; Cl ; O=C(O)c1ccnc(OC2CCNCC2)c1]

To 2-((1-(tert-butoxycarbonyl)piperidin-4-yl)oxy)isonicotinic acid I30 (1.76 g, 4.15 mmol, 1 equiv) in 1,4-dioxane (10 mL) was added a 4M solution of HCl in 1,4-dioxane (1.35 mL, 5.39 mmol, 1.3 equiv). The reaction was stirred at room temperature for 16 hours then dried in vacuo to give a solid. The solid was re-suspended in 1,4-dioxane (10 mL) and a 4M solution of HCl in 1,4-dioxane (4 mL, 16 mmol, 3.9 equiv) was added. The reaction was stirred at room temperature for an additional 2 days, and then the solvent was evaporated to give the title compound (1.23 g @ 100% conversion) as a solid. LCMS-B (hydrophilic method): Retention Time 0.56 min, m/z 221.1 [M−H]⁻ for the free base.

### (xxi) 2-((1-(Isopropoxncarbonyl)piperidin-4-yl)oxy)isonicotinic acid (I50) and 2-((1-(Isobutoxycarbonyl)piperidin-4-yl)oxy)isonicotinic acid (I51)

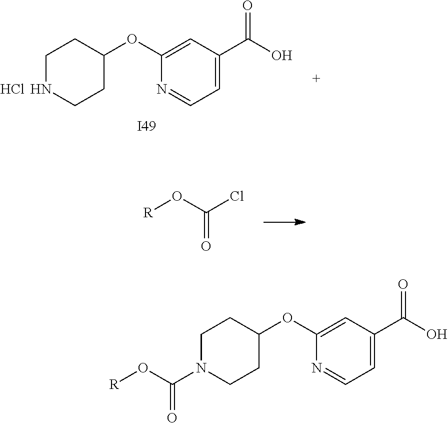

General Method:

The desired chloroformate (0.87 mmol, 2 equiv) was added drop-wise to a mixture of 2-(piperidin-4-yloxy)isonicotinic acid dihydrochloride I49 (129 mg, 0.437 mmol, 1 equiv) and sodium hydroxide (73 mg, 1.8 mmol, 4.2 equiv) in water (10 mL). The reaction was stirred at ambient temperature overnight. The pH was adjusted to pH=3 with a 0.5 M aqueous solution of citric acid and the aqueous phase was extracted with EtOAc (3×20 mL). The combined organic layers were washed with water (20 mL), brine (20 mL), dried over Na₂SO₄ and concentrated in vacuo. The resultant oil was purified by column chromatography (12 g SiO₂ cartridge, 0-65% EtOAc in petroleum benzine 40-60° C.) to give the title compound.

| Intermediate | Name and Structure | Yield and Analytical data |
|---|---|---|
| 150 | 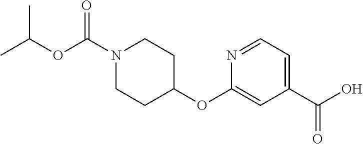 [SMILES: CC(C)OC(=O)N1CCC(Oc2cc(C(=O)O)ccn2)CC1] 2-((1-(Isopropoxycarbonyl)piperidin- 4-yl)oxy)isonicotinic acid | 114 mg, 85% yield LCMS-B: Retention Time 3.21 min, m/z 309.1 [M + H]⁺, 331.1 [M + Na]⁺. |
| 151 | 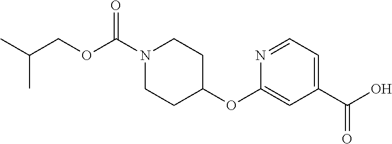 [SMILES: CC(C)COC(=O)N1CCC(Oc2cc(C(=O)O)ccn2)CC1] 2-((1-(Isobutoxycarbonyl)piperidin-4- yl)oxy)isonicotinic acid | 46 mg, 16% yield LCMS-B: Retention Time 3.31 min, m/z 323.1 [M + H]⁺, 345.1 [M + Na]⁺. |

### (xxii) Lithium 4-(4-acetylpiperazine-1-carbonyl)benzoate (I55)

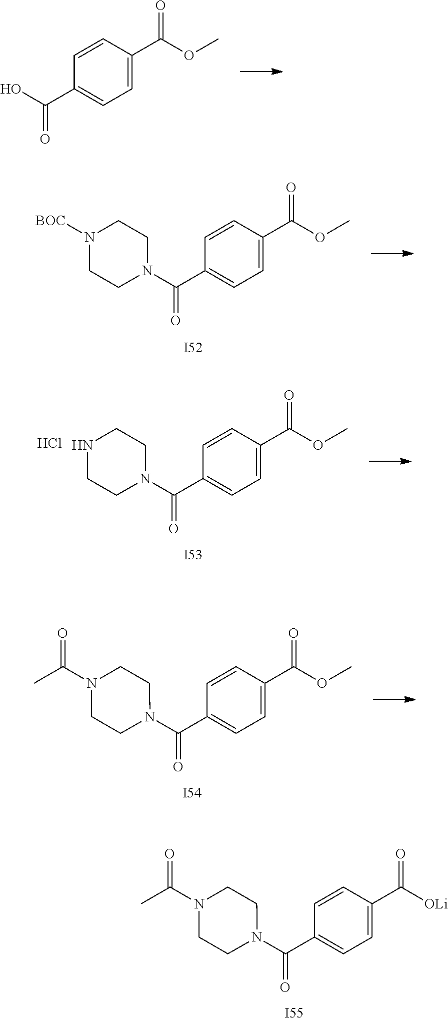

### (a) tert-Butyl 4-(4-(methoxycarbonyl)benzoyl)piperazine-1-carboxylate I52

4-(Methoxycarbonyl)benzoic acid (500 mg, 2.78 mmol), DCM (25 mL), DMAP (17 mg, 5 mol %), triethylamine (1.16 mL, 8.33 mmol), tert-butyl piperazine-1-carboxylate (569 mg, 3.05 mmol) and EDCI HCl (585 mg, 3.05 mmol) were stirred at 30° C. overnight. The mixture was diluted with DCM (50 mL) and added to 10% w/v aqueous NaHSO₄ (150 mL). The organic phase was separated, and the aqueous phase extracted with further DCM (50 mL). The pooled organic extracts were washed with water (100 mL), brine (100 mL), dried over Na₂SO₄ and concentrated in vacuo. Chromatography (12 g silica cartridge, 0-100% ethyl acetate/hexanes) gave the title compound as a solid (613 mg, 63% yield). ¹H NMR (400 MHz, CDCl₃) δ 8.09 (d, J=8.2 Hz, 2H), 7.46 (d, J=8.2 Hz, 2H), 3.94 (s, 3H), 3.74 (br s, 2H), 3.57-3.27 (m, 6H), 1.47 (s, 9H). LCMS-B: Retention Time 3.25 min; m/z 371.1 [M+Na]⁺; 293.1 [M-^{t}Bu+2H]⁺; 249.2 [M−Boc+2H]⁺

### (b) Methyl 4-(piperazine-1-carbonyl)benzoate hydrochloride I53

tert-Butyl 4-(4-(methoxycarbonyl)benzoyl)piperazine-1-carboxylate I52 (611 mg, 1.75 mmol) was dissolved in 1,4-dioxane (5 mL) and a 4.0M solution of HCl in 1,4-dioxane (5 mL) was added. The mixture was stirred at 30° C. for 2 hours then concentrated in vacuo to give the title compound as a solid (511 mg). LCMS-B: Retention Time 0.95 min; m/z 249.1 [M+H]⁺ for free base.

### (c) Methyl 4-(4-acetylpiperazine-1-carbonyl)benzoate I54

Methyl 4-(piperazine-1-carbonyl)benzoate hydrochloride I53 (100 mg, 0.35 mmol), DCM (1.5 mL), triethylamine (0.147 mL, 1.05 mmol), DMAP (0.4 mg, 1 mol %) and acetyl chloride (0.050 mL, 0.70 mmol) were stirred at 30° C. After 17 hours, the mixture was quenched with 10% w/v aqueous NaHSO₄ (1 mL), the organic phase separated and loaded onto a 4 g silica cartridge. Chromatography (0-100% ethyl acetate/hexanes) gave the title compound as a solid (54 mg, 53% yield). LCMS-B: Retention Time 2.92 min; m/z 291.1 [M+H]⁺

### (d) Lithium 4-(4-acetylpiperazine-1-carbonyl)benzoate I55

Methyl 4-(4-acetylpiperazine-1-carbonyl)benzoate I54 (54 mg, 0.19 mmol) and lithium hydroxide monohydrate (8.6 mg, 0.21 mmol) were stirred in THF (1 mL), water (0.5 mL) and methanol (0.5 mL). After 4 hours, the mixture was concentrated in vacuo, the residue diluted with absolute ethanol (5 mL) and again concentrated in vacuo to give the title compound as a solid (54 mg). The solid was used without further characterisation or purification.

### (xxiii) Lithium 4-(4-(methoxycarbonyl)piperazine-1-carbonyl)benzoate I57

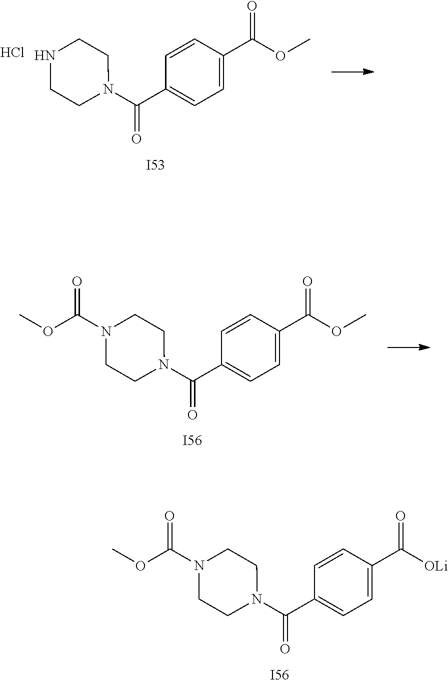

### (a) Methyl 4-(4-(methoxycarbonyl)benzoyl)piperazine-1-carboxylate I56

Methyl 4-(piperazine-1-carbonyl)benzoate hydrochloride I53 (100 mg, 0.35 mmol), DCM (1.5 mL), triethylamine (0.147 mL, 1.05 mmol), DMAP (0.4 mg, 1 mol %) and methyl chloroformate (0.054 mL, 0.70 mmol) were stirred at 30° C. After 17 hours the mixture was quenched with 10% w/v aqueous NaHSO₄ (1 mL), the organic phase separated and loaded onto a 4 g silica cartridge. Chromatography (0-100% ethyl acetate/hexanes) gave the title compound as a solid (58 mg, 54% yield). LCMS-B: Retention Time 3.03 min; m/z 307.1 [M+H]⁺

### (b) Lithium 4-(4-(methoxycarbonyl)piperazine-1-carbonyl)benzoate I57

Methyl 4-(4-(methoxycarbonyl)benzoyl)piperazine-1-carboxylate I56 (58 mg, 0.19 mmol) and lithium hydroxide monohydrate (8.7 mg, 0.21 mmol) were stirred in THF (1 mL), water (0.5 mL) and methanol (0.5 mL). After 4 hours, the mixture was concentrated in vacuo, the residue diluted with absolute ethanol (5 mL) and again concentrated in vacuo to give the title compound as a solid (56 mg). The solid was used without further characterisation or purification.

### (xxiv) 2-((1-Acetylpiperidin-4-yl)oxy)benzoic acid I58

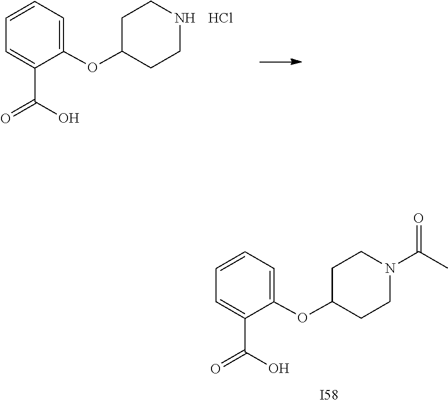 [SMILES: O=C(O)c1ccccc1OC1CCNCC1 ; Cl ; "I58" ; CC(=O)N1CCC(Oc2ccccc2C(=O)O)CC1]

2-(Piperidin-4-yloxy)benzoic acid hydrochloride (200 mg, 0.78 mmol), DCM (5 mL), triethylamine (0.541 mL, 3.88 mmol) and acetyl chloride (0.221 mL, 3.10 mmol) were stirred at 30° C. After 3 hours, DCM (5 mL) and water (10 mL) were added and the mixture adjusted to pH 1 with 3M aqueous HCl. The organic layer was separated (phase separator cartridge) and concentrated in vacuo. Chromatography (12 g silica cartridge, 0-100% ethyl acetate/hexanes, then 0-20% methanol/ethyl acetate) gave the title compound as solid (173 mg, 85% yield). LCMS-A: Retention Time 5.24 min; m/z 264.2 [M+H]⁺

### (xxv) Lithium 4-(3-oxa-8-azabicyclo[3.2.1]octane-8-carbonyl)-2-ethoxy-5-fluorobenzoate I61

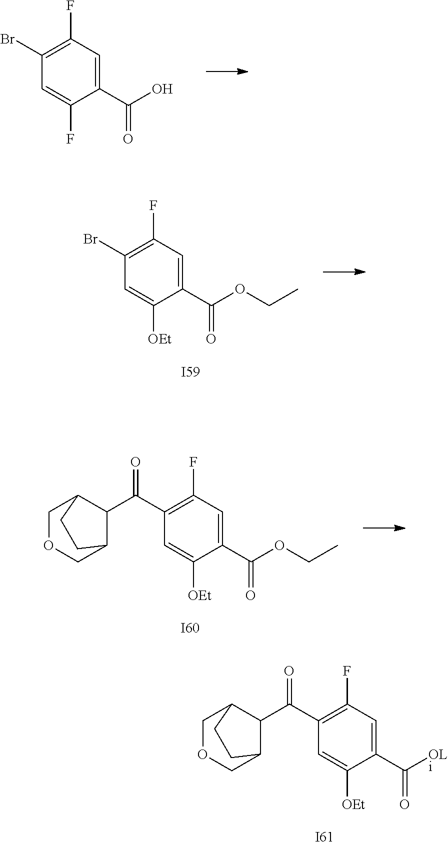

### (a) Ethyl 4-bromo-2-ethoxy-5-fluorobenzoate I59

4-Bromo-2,5-difluorobenzoic acid (1.00 g, 4.22 mmol) was suspended in DCM (20 mL) and cooled to 0° C. Oxalyl chloride (0.543 mL, 6.33 mmol) and DMF (1 drop) were added and the mixture stirred at room temperature. After one hour, the mixture was concentrated in vacuo, the residue dissolved in DCM (10 mL) and cooled to 0° C. Absolute ethanol (10 mL) was added and the mixture stirred at room temperature. After 15 minutes, the mixture was concentrated in vacuo and the residue taken up in absolute ethanol. Sodium metal (146 mg, 6.33 mmol) was dissolved in absolute ethanol (30 mL) and the solution added to the solution of the ester. After 2 hours, the reaction had a thick precipitate, the mixture was diluted with dry THF (50 mL) and the stir bar replaced with an oversized stir bar. After 19 hours, the mixture was concentrated in vacuo, the residue slurried in ethyl acetate (75 mL) and filtered. The filtrate was concentrated in vacuo and purified by chromatography (12 g silica cartridge, 0-10% ethyl acetate/hexanes) to give the title compound (216 mg, 18% yield) as a solid. ¹H NMR (400 MHz, CDCl₃) δ 7.57 (d, J=8.6 Hz, 1H), 7.13 (d, J=5.5 Hz, 1H), 4.35 (q, J=7.2 Hz, 2H), 4.07 (q, J=7.0 Hz, 2H), 1.46 (t, J=7.0 Hz, 3H), 1.37 (t, J=7.1 Hz, 3H). LCMS-B: Retention Time 3.53 min, m/z 245.0 [M−OEt]⁺ for ⁷⁹Br

### (b) Ethyl 4-(3-oxa-8-azabicyclo[3.2.1]octane-8-carbonyl)-2-ethoxy-5-fluorobenzoate I60

Palladium(II) acetate (8 mg, 5 mol %), xantphos (21 mg, 5 mol %), sodium carbonate (232 mg, 2.19 mmol), 3-oxa-8-azabicyclo[3.2.1]octane hydrochloride salt (163 mg, 1.09 mmol) and ethyl 4-bromo-2-ethoxy-5-fluorobenzoate I59 (212 mg, 0.728 mmol) were stirred in toluene (2 mL) in a schlenk tube under nitrogen. The tube was flushed with carbon monoxide, and heated to 80° C. under carbon monoxide atmosphere. After 18 hours, the mixture was cooled to room temperature and diluted with ethyl acetate (20 mL). The mixture was filtered through Celite and the filtrate concentrated in vacuo. Chromatography (12 g silica cartridge, 0-60% ethyl acetate/hexanes) gave the title compound (41 mg, 16% yield) as a film. ¹H NMR (400 MHz, CDCl₃) δ 7.51 (d, J=9.2 Hz, 1H), 7.01 (d, J=5.1 Hz, 1H), 4.75 (d, J=5.8 Hz, 1H), 4.36 (q, J=7.1 Hz, 2H), 4.10 (q, J=7.0 Hz, 2H), 3.84 (d, J=11.0 Hz, 1H), 3.73-3.68 (m, 2H), 3.64 (d, J=10.9 Hz, 1H), 3.56 (dd, J=11.0, 1.6 Hz, 1H), 2.13-1.90 (m, 4H), 1.45 (t, J=7.0 Hz, 3H), 1.38 (t, J=7.1 Hz, 3H). LCMS-B: Retention Time 3.23 min; m/z 352.1 [M+H]⁺

### (c) Lithium 4-(3-oxa-8-azabicyclo[3.2.1]octane-8-carbonyl)-2-ethoxy-5-fluorobenzoate I61

A solution of ethyl 4-(3-oxa-8-azabicyclo[3.2.1]octane-8-carbonyl)-2-ethoxy-5-fluorobenzoate I60 (41 mg, 0.12 mmol) in THF (1 mL) was stirred vigorously, and a solution of lithium hydroxide monohydrate (7.3 mg, 0.18 mmol) in water (0.5 mL) was added. After 4.5 hours, the mixture was concentrated in vacuo, the residue dissolved in absolute ethanol and the mixture again concentrated in vacuo to give the title compound. LCMS-B: Retention Time 2.96 min; m/z 324.1 [M-Li+2H]⁺; m/z 322.1 [M-Li]⁻

### (xxvi) 6-(3-Oxa-8-azabicyclo[3.2.1]octane-8-carbonyl)-2-ethoxnnicotinic acid I65

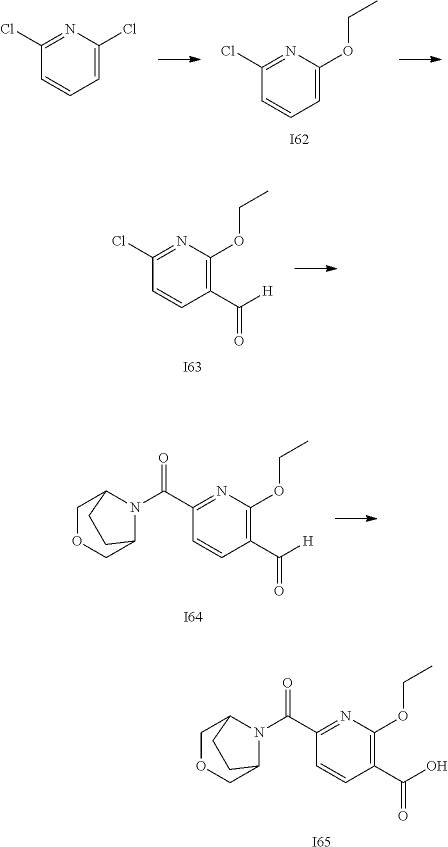

### (a) 2-Chloro-6-ethoxypyridine I62

To a solution of 2,6-dichloropyridine (5.0 g, 33.8 mmol) in EtOH (50 mL) was added EtONa (9.2 g, 0.14 mol). The mixture was stirred at 60° C. for 24 hours. The solvent was removed, and the residue obtained was dissolved in water (100 mL). The aqueous layer was acidified to pH 7 with 2 M HCl, then extracted with DCM (100 mL×2). The combined organic layers were washed with water (100 mL) and brine (100 mL), dried (Na₂SO₄) and concentrated to give the title compound as oil (4.2 g, 79%). LCMS-C: Retention Time 2.64 min; m/z 158.1 [M+H]⁺.

### (b) 6-Chloro-2-ethoxynicotinaldehyde I63

To a solution of 2-chloro-6-ethoxypyridine I62 (2.0 g, 12.7 mmol) in THF (40 mL) at −78° C. was added t-BuLi (1.6 M in pentane, 8.8 mL, 14.0 mmol) dropwise under N₂. After stirring at the same temperature for 1 hour, DMF (2.8 g, 38.1 mmol) was added dropwise and the reaction mixture was stirred at −78° C. for 30 minutes then warmed to room temperature and stirred for 30 minutes. The reaction mixture was quenched with 2M HCl (5 mL) and extracted with EtOAc (50 mL×2). The combined organic layers were washed with water (50 mL×3) and brine (50 mL), dried (Na₂SO₄) and concentrated to give the title compound as oil (2.0 g, 87%). ¹HNMR (400 MHz, DMSO-d6) δ 10.17 (s, 1H), 8.09 (d, J=8.0 Hz, 1H), 7.23 (d, J=8.0 Hz, 1H), 4.44 (q, J=14.0, 7.2 Hz, 2H), 1.38 (t, J=7.2 Hz, 3H). LCMS-C: Retention Time 2.64 min; m/z 186 [M+H]⁺; 218.1 [M+MeOH+H]⁺.

### (c) 6-(3-Oxa-8-azabicyclo[3.2.1]octane-8-carbonyl)-2-ethoxynicotinaldehyde I64

To a solution of 3-oxa-8-azabicyclo[3.2.1]octane hydrochloride (1.5 g, 10.3 mmol) in toluene (80 mL) was added Et₃N (2.6 g, 25.8 mmol), Xantphos (0.2 g, 0.34 mmol) and Pd(OAc)₂ (40 mg, 0.17 mmol). The mixture was degassed three times under N₂ followed by addition of 6-chloro-2-ethoxynicotinaldehyde I63 (1.6 g, 8.6 mmol). The mixture was degassed three times under N₂ and then three times under CO. The reaction was stirred at 90° C. overnight. Water (80 mL) was added and the mixture was extracted with EtOAc (80 mL×2), the combined organic layers were washed with water (80 mL), brine (80 mL), dried (Na₂SO₄) and concentrated. The residue was purified by column chromatography (100% petroleum ether to 50% EtOAc in petroleum ether) to give the title compound as a solid (1.1 g, 44%): LCMS-C: Retention Time 2.22 min; m/z 291.1 [M+H]⁺.

### (d) 6-(3-Oxa-8-azabicyclo[3.2.1]octane-8-carbonyl)-2-ethoxynicotinic acid I65

To a mixture of 6-(3-oxa-8-azabicyclo[3.2.1]octane-8-carbonyl)-2-ethoxynicotinaldehyde I64 (100 mg, 0.34 mmol) in a mixture of t-BuOH (5 mL) and 2-methylbut-2-ene (2 mL) was added a solution of NaH₂PO₄ 2H₂O (376 mg, 2.4 mmol) and NaClO₂ (300 mg, 3.3 mmol) in water (5 mL). The resulting mixture was stirred at room temperature for 2 hours after which the solvent was removed under reduced pressure. The residue obtained was dissolved in water and the aqueous layer was acidified to pH 5 with 2 M HCl and extracted with EtOAc (20 mL×4). The combined organic layers were dried (Na₂SO₄) and concentrated to give the title compound as a solid (80 mg, 76%). LCMS-C: Retention Time 1.74 min; m/z 307.1 [M+H]⁺.

### (xxvii) 6-(2-Oxo-2-(piperidin-1-yl)ethyl)nicotinic acid I70

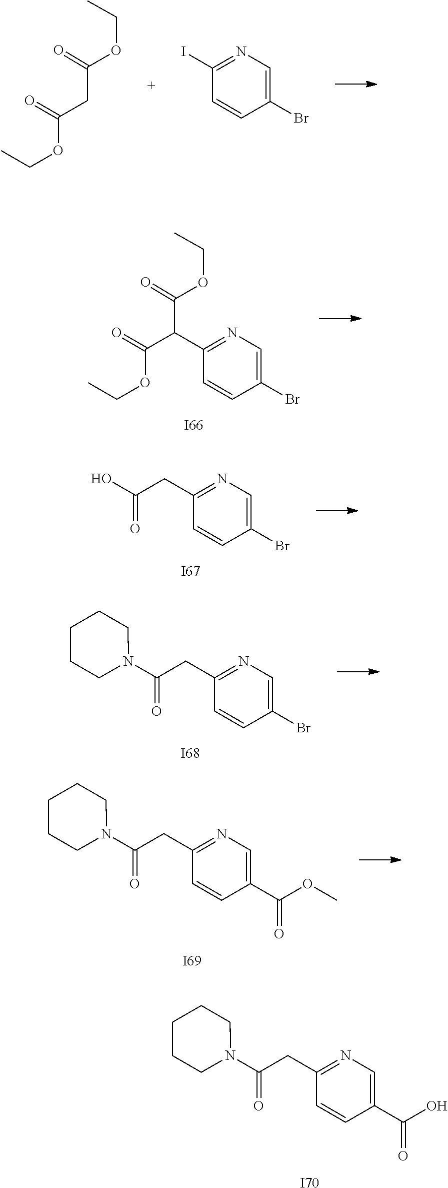

### (a) Diethyl 2-(5-bromopyridin-2-yl)malonate I66

To a solution of 5-bromo-2-iodopyridine (18.0 g, 63.4 mmol) in 1,4-dioxane (100 mL) was added diethyl malonate (20.3 g, 126.8 mmol), Cs₂CO₃ (62.0 g, 190.2 mmol), CuI (1.2 g, 6.3 mmol) and picolinic acid (1.6 g, 12.7 mmol). The resulting mixture was heated to 70° C. and stirred overnight. Water (100 mL) and EtOAc (100 mL) were added and the aqueous layer extracted with EtOAc (3×50 mL). The combined organic extracts were washed with water (3×50 mL) and brine (3×50 mL), dried (Na₂SO₄) and concentrated. The crude product was purified by silica gel chromatography (ethyl acetate/petroleum ether=1/20) to give the title compound as oil (19.6 g, 97%). LCMS-C: Retention Time 2.49 min; m/z 316.0, 318.0 [M+H]⁺.

### (b) 2-(5-Bromopyridin-2-yl)acetic acid I67

To a solution of diethyl 2-(5-bromopyridin-2-yl)malonate I66 (2.1 g, 6.6 mmol) in MeOH (30 mL) was added 2 M NaOH aqueous solution (14 mL, 28 mmol). The resulting mixture was stirred at room temperature for 3 hours. The solution was concentrated under reduced pressure and the residue was dissolved in water (20 mL). The pH of the solution was adjusted to 3-4 by addition of 6 M HCl. The precipitate formed was collected by filtration, washed with water and dried at 50° C. to give the title compound as a solid (1.0 g, 70%). LCMS-C: Retention Time 0.78 min; m/z 216.0 [M+H]⁺.

### (c) 2-(5-Bromopyridin-2-yl)-1-(piperidin-1-yl)ethanone I68

To a solution of 2-(5-bromopyridin-2-yl)acetic acid I67 (890 mg, 4.1 mmol) in DCM (5 mL) was added HATU (2.4 g, 6.2 mmol) followed by a solution of piperidine (526 mg, 6.2 mmol) in DCM (5 mL) and DIPEA (1.9 mL, 14.4 mmol). The resulting mixture was stirred at room temperature overnight. Water (15 mL) and DCM (15 mL) were then added, the layers were separated and the aqueous layer extracted with DCM (3×15 mL). The combined organic extracts were washed with saturated NaHCO₃ (3×10 mL) and brine (3×10 mL), dried (Na₂SO₄) and concentrated in vacuo. The residue was purified by silica gel chromatography (ethyl acetate/petroleum ether=1/10) to give the title compound as a solid (960 mg, 82%). ¹HNMR (400 MHz, DMSO-d₆) δ 8.59 (d, J=2.4 Hz, 1H), 7.97 (dd, J=8.4, 2.4 Hz, 1H), 7.28 (d, J=8.4 Hz, 1H), 3.83 (s, 2H), 3.45-3.40 (m, 4H), 1.58-1.52 (m, 2H), 1.43-1.37 (m, 4H); LCMS-C: Retention Time 2.22 min; m/z 283.1 [M+H]⁺.

### (d) Methyl 6-(2-oxo-2-(piperidin-1-yl)ethyl)nicotinate I69

To a solution of 2-(5-bromopyridin-2-yl)-1-(piperidin-1-yl)ethanone I68 (500 mg, 1.8 mmol) in MeOH (20 mL) was added Pd(dppf)Cl₂ (65 mg, 0.1 mmol) and triethylamine (394 mg, 3.9 mmol). The resulting mixture was heated at reflux under a carbon monoxide atmosphere overnight. The mixture was concentrated, and the residue was purified by silica gel chromatography (ethyl acetate/petroleum ether=1/1) to give the title compound as a solid (417 mg, 90%). LCMS-C: Retention Time 1.62 min; m/z 263.1 [M+H]⁺.

### (e) 6-(2-Oxo-2-(piperidin-1-yl)ethyl)nicotinic acid I70

To a solution of methyl 6-(2-oxo-2-(piperidin-1-yl)ethyl)nicotinate I69 (390 mg, 1.5 mmol) in a mixture of THF (5 mL), MeOH (2 mL) and water (2 mL) was added lithium hydroxide monohydrate (313 mg, 7.4 mmol). The resulting mixture was stirred at room temperature overnight. The organic solvent was removed and the aqueous layer neutralized with 4 M HCl aqueous solution to a pH of 5-6. The water was removed to give the title crude product which was dissolved in a mixed solution of MeOH/DCM (1/20) and filtered. The filtrate was concentrated in vacuo to give the title compound as an oil (338 mg, 91%). The material was carried forward without further purification. LCMS-C: Retention Time 0.55 min; m/z 249.1 [M+H]⁺.

### (xxviii) 2-(3,6-Dihydro-2H-pyran-4-yl)-4,4,5,5-tetramethyl-1,3,2-dioxaborolane I71

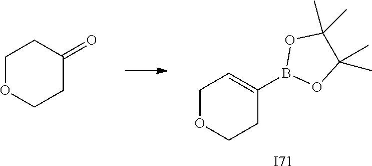

To a solution of diisopropylamine (1.41 g, 13.98 mmol) in THF (10 mL) at −20° C. under N₂ was added dropwise n-BuLi (5.82 mL, 13.98 mmol). The mixture was stirred for 30 minutes at −20° C. then cooled to −70° C. and a solution of dihydro-2H-pyran-4(3H)-one (2.0 g, 9.99 mmol) in THF (5 mL) was added. The reaction was stirred at −70° C. for 30 minutes. A solution of 1,1,1-trifluoro-N-phenylmethanesulfonamide (3.57 g, 9.99 mmol) in THF (5 mL) was added dropwise, the resulting mixture was allowed to warm to room temperature and stirred for 18 hours. The reaction mixture was concentrated under reduced pressure and the residue obtained diluted with water (20 mL). The mixture was extracted with ethyl acetate (3×20 mL) and the combined organic layers dried (Na₂SO₄), filtered and concentrated. The residue obtained was purified by silica gel chromatography (ethyl acetate/petroleum ether=1/20) to give the triflate intermediate. To a solution of 4,4,4′,4′,5,5,5′,5′-octamethyl-2,2′-bi(1,3,2-dioxaborolane) (993 mg, 3.91 mmol) in DMSO (5 mL) under N₂ was added KOAc (959.8 mg, 9.78 mmol) and Pd(dppf)Cl₂ (71.7 mg, 0.097 mmol). A solution of the triflate intermediate (750 mg) in DMSO (1 mL) was added and the reaction heated at 80° C. overnight. The mixture was diluted with water (20 mL) and extracted with DCM (3×20 mL). The combined organic layers were washed with saturated NaHCO₃ (3×10 mL) and brine (3×10 mL), dried (Na₂SO₄) and concentrated. The residue obtained was purified by silica gel chromatography (ethyl acetate/petroleum ether=1/2) to give the title compound (300 mg) as a solid which was used without further purification or analysis.

### (xxix) 2-(Tetrahydro-2H-pyran-4-yl)isonicotinic acid I75

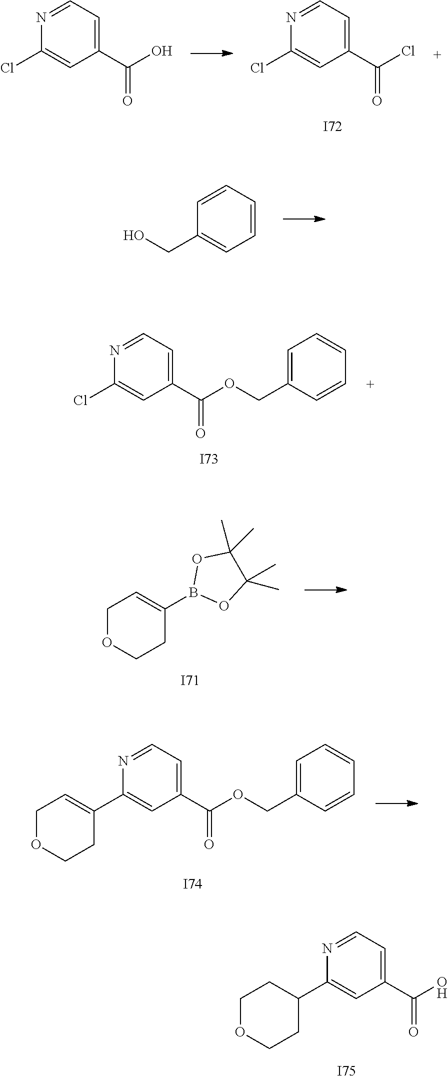

### (a) 2-Chloroisonicotinoyl chloride I72

A solution of 2-chloroisonicotinic acid (2.0 g, 12.69 mmol) in thionyl chloride (15 mL) was heated at 80° C. for 3 hours. The mixture was concentrated in vacuo and the residue resuspended in DCM (3×10 mL) and concentrated to remove the excess thionyl chloride to give the title compound as oil (2.2 g, 100%). LCMS-C: (MeOH quench) Retention Time 1.79 min; m/z 172.1 [M+H]⁺.

### (b) Benzyl 2-chloroisonicotinate I73

To a solution of benzyl alcohol (1.37 g, 12.69 mmol) in DCM (80 mL) was added triethylamine (5.07 g, 50.76 mmol) followed by dropwise addition of 2-chloroisonicotinoyl chloride I72 (2.2 g, 12.69 mmol), and the reaction was stirred at room temperature overnight. The mixture was diluted with water (50 mL) and extracted with DCM (3×50 mL). The combined organic layers were washed with saturated NaHCO₃ (3×10 mL), brine (3×10 mL), dried (Na₂SO₄) and concentrated to give the title compound (3.1 g, 99%) as oil. LCMS-C: Retention Time 2.88 min; m/z 248.1 [M+H]⁺.

### (c) Benzyl 2-(3,6-dihydro-2H-pyran-4-yl)isonicotinate I74

To a solution of benzyl 2-chloroisonicotinate I73 (319 mg, 1.29 mmol) in a mixture of toluene (2 mL) and THF (10 mL) was added 2-(3,6-dihydro-2H-pyran-4-yl)-4,4,5,5-tetramethyl-1,3,2-dioxaborolane I71 (300 mg, 1.42 mmol) and 2M Na₂CO₃ aqueous solution (1.29 mL). Pd(dppf)Cl₂ (38 mg, 0.05 mmol) was added and the reaction heated at 100° C. for 3 hours. The mixture was diluted with 1M HCl (20 mL) and extracted with DCM (3×20 mL), the combined organic layers were washed with saturated NaHCO₃ (3×10 mL), brine (3×10 mL), dried (Na₂SO₄) and concentrated. The residue was purified by silica gel chromatography (ethyl acetate/petroleum ether=1/5) to give the title compound (60 mg, 6%) as an oil. LCMS-C: Retention Time 2.88 min; m/z 296.2 [M+H]⁺.

### (d) 2-(Tetrahydro-2H-pyran-4-yl)isonicotinic acid I75

To a solution of benzyl 2-(3,6-dihydro-2H-pyran-4-yl)isonicotinate I74 (60 mg, 0.20 mmol) in MeOH (5 mL) was added 10% Pd/C (20 mg). The mixture was stirred under H₂ at room temperature for 1 day. The mixture was filtered and the filtrate concentrated to give the title compound as an oil (30 mg, 71%). LCMS-C: Retention Time 0.52 min; m/z 208.2 [M+H]⁺.

### (xxx) 4-(2-(3-Oxa-8-azabicyclo[3.2.1]octan-8-yl)-2-oxoethyl)-2-ethoxybenzoic acid I79

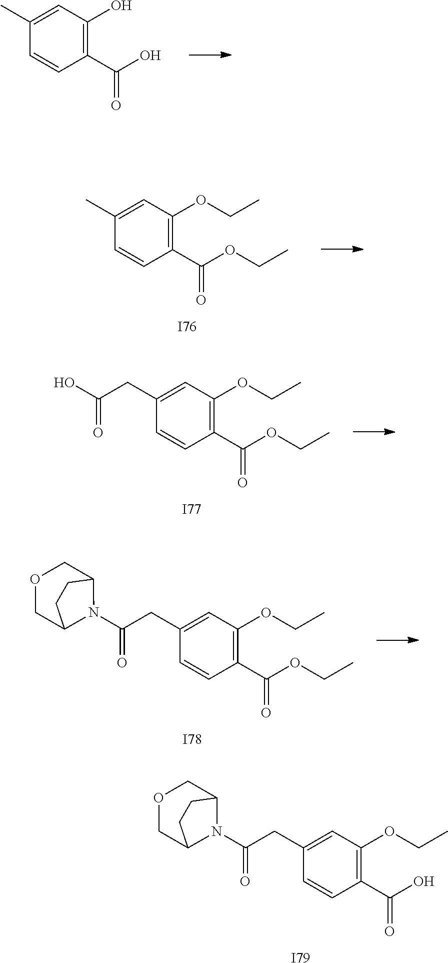

### (a) Ethyl 2-ethoxy-4-methylbenzoate I76

To a mixture of 2-hydroxy-4-methylbenzoic acid (20.0 g, 131.5 mmol) and K₂CO₃ (54.5 g, 394.5 mmol) in DMSO (50 mL) at 40° C. was added bromoethane (21.5 g, 197.2 mmol) over 30 minutes. The reaction was stirred at 40° C. for 2 hours. Further bromoethane (21.5 g, 197.2 mmol) was then added dropwise and the reaction stirred at 40° C. for 8 hours. DCM (200 mL) was added and the mixture was filtered. The organic layer was washed with water (4×150 mL), dried (Na₂SO₄) and concentrated to give the title compound (24.1 g, 88%) as a clear oil. LCMS-C: Retention Time 2.77 min; m/z 209.1[M+H]⁺.

### (b) 2-(3-Ethoxy-4-(ethoxycarbonyl)phenyl)acetic acid I77

To a solution of diisopropylamine (1.46 g, 14.4 mmol) in anhydrous THF (20 mL) at −30° C. was added n-BuLi (2.4M in hexane, 5.8 mL, 14.4 mmol) slowly under a nitrogen atmosphere. The mixture was stirred at −30° C. for 30 minutes then cooled to −78° C. and HMPA (4.0 g) was added. A solution of ethyl 2-ethoxy-4-methylbenzoate I76 (2.0 g, 9.6 mmol) in anhydrous THF (5 mL) was then added dropwise and the resulting mixture stirred at −78° C. for 2 hours. CO₂ (g) was bubbled through the mixture until the colour of the anion discharged then for a further 30 minutes. The reaction was allowed to warm to 10° C., then diluted with water (20 mL) and extracted with diethyl ether (2×20 mL). The aqueous layer was acidified to pH 2 by addition of 10% aqueous H₂SO₄ and extracted with DCM (2×20 mL). The combined DCM layers were washed with water and brine, dried (Na₂SO₄) and concentrated to give the title compound (550 mg, 23%) as oil. LCMS-C: Retention Time 2.36 min; m/z 253.1[M+H]⁺.

### (c) Ethyl 4-(2-(3-oxa-8-azabicyclo[3.2.1]octan-8-yl)-2-oxoethyl)-2-ethoxybenzoate I78

A mixture of 2-(3-ethoxy-4-(ethoxycarbonyl)phenyl)acetic acid I77 (550 mg, 2.2 mmol), 3-oxa-8-azabicyclo[3.2.1]octane hydrochloride salt (320 mg, 2.1 mmol), EDCI (500 mg, 2.6 mmol), HOBt (30 mg, 0.22 mmol) and DIPEA (710 mg, 5.5 mmol) in DCM (10 mL) was stirred at room temperature overnight. Water (20 mL) was added and the reaction mixture extracted with DCM (3×10 mL), the organic layers were combined and washed with saturated aqueous NaHCO₃, water and brine, dried (Na₂SO₄) and concentrated to give the title product (710 mg, 93%) as an oil. LCMS-C: Retention Time 2.38 min; m/z 348.2 [M+H]⁺.

### (d) 4-(2-(3-Oxa-8-azabicyclo[3.2.1]octan-8-yl)-2-oxoethyl)-2-ethoxybenzoic acid I79

To a solution of ethyl 4-(2-(3-oxa-8-azabicyclo[3.2.1]octan-8-yl)-2-oxoethyl)-2-ethoxybenzoate I78 (710 mg, 2.0 mmol) in a mixture of THF (5 mL), MeOH (10 mL) and water (5 mL) was added LiOH.H₂O (429 mg, 10.2 mmol). The reaction was stirred at room temperature overnight then the solvent was removed in vacuo. The residue obtained was re-dissolved in water (10 mL) then acidified to pH 2 by addition of 10% aqueous H₂SO₄ solution. The resulting mixture was extracted with DCM (3×10 mL) and the combined organic layers dried (Na₂SO₄) and concentrated to give the title compound (540 mg, 85%) as a solid. LCMS-C: Retention Time 1.20 min; m/z 320.2 [M+H]⁺.

### (xxxi) 5-Chloro-2-((tetrahydro-2H-pyran-4-yl)oxy)isonicotinic acid I81

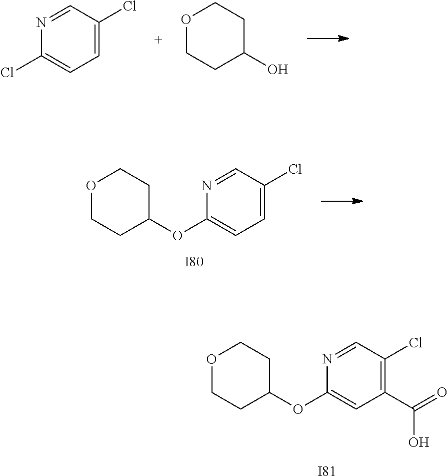 [SMILES: Clc1ccc(Cl)nc1 ; OC1CCOCC1 ; "I80" ; Clc1ccc(OC2CCOCC2)nc1 ; "I81" ; O=C(O)c1cc(OC2CCOCC2)ncc1Cl]

### (a) 5-Chloro-2-((tetrahydro-2H-pyran-4-yl)oxy)pyridine I80

To a solution of tetrahydro-2H-pyran-4-ol (1.0 g, 10 mmol) in DMF (25 mL) at 0° C. was added NaH in mineral oil (60% wt, 1.2 g, 30 mmol), and the mixture was stirred at 0° C. for 30 minutes. 2,5-Dichloropyridine (1.8 g, 12 mmol) was added and the resulting mixture was heated at 60° C. for 2 hours, then cooled to room temperature and allowed to stir overnight. The reaction was partitioned between EtOAc (50 mL) and water and the organic fraction washed with water (3×50 mL) and brine, dried (Na₂SO₄) and concentrated. The crude material obtained was purified by silica gel chromatography (2% EtOAc in petroleum ether) to give the title compound (1.3 g, 62%). LCMS-C: Retention Time 2.65 min; m/z 214.1 [M+H]⁺.

### (b) 5-Chloro-2-((tetrahydro-2H-pyran-4-yl)oxy)isonicotinic acid I81

To a solution of diisopropylamine (284 mg, 2.81 mmol) in anhydrous THF (20 mL) at −5° C. was added n-BuLi (2.4M in hexane, 1.2 mL, 2.81 mmol) slowly under a nitrogen atmosphere. The mixture was then stirred at −15° C. to −5° C. for 30 minutes, then cooled to −78° C. and a solution of 5-chloro-2-((tetrahydro-2H-pyran-4-yl)oxy)pyridine I80 (500 mg, 2.34 mmol) in anhydrous THF (5 mL) was added dropwise. The resulting mixture was stirred at −78° C. for 2 hours and then CO₂ (g) was bubbled through the mixture for 15 minutes. Water (10 mL) was added and the mixture extracted with diethyl ether (×2). The aqueous layer was separated and acidified to pH 3 by addition of 10% aqueous H₂SO₄, the aqueous layer was extracted with DCM (×2) and the combined organic layers washed with water and brine, dried (Na₂SO₄) and concentrated to give the title compound (240 mg, 40%) as a solid. LCMS-C: Retention Time 1.77 min; m/z 258.2[M+H]⁺.

### (xxxii) Lithium 6-((1-(methoxycarbonyl)piperidin-4-yl)oxy)pyrimidine-4-carboxylate I86

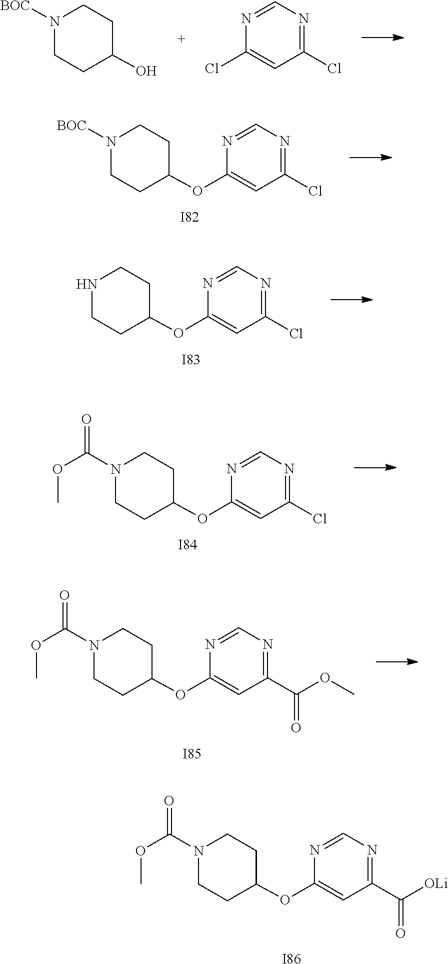

### (a) tert-Butyl 4-((6-chloropyrimidin-4-yl)oxy)piperidine-1-carboxylate I82

To a solution of tert-butyl 4-hydroxypiperidine-1-carboxylate (3.2 g, 16.1 mmol) in THF (45 mL) was added NaH in mineral oil (60% wt, 0.97 g, 24.2 mmol), and the mixture was stirred at 60° C. for 1 hour, then cooled to room temperature and a solution of 4,6-dichloropyrimidine (2.0 g, 13.4 mmol) in THF (15 mL) was added. The resulting mixture was then stirred at room temperature for 2 hours. Water (50 mL) was added and the reaction mixture was extracted with EtOAc (×2). The combined organic layers were washed with brine, dried (Na₂SO₄) and concentrated to give the title compound (4.3 g, 100%) as an oil. LCMS-C: Retention Time 2.95 min; m/z 314.1 [M+H]⁺.

### (b) 4-Chloro-6-(piperidin-4-yloxy)pyrimidine dihydrochloride I83

A solution of tert-butyl 4-((6-chloropyrimidin-4-yl)oxy)piperidine-1-carboxylate I82 (4.3 g, 13.4 mmol) in HCl/EtOAc (4 M, 40 mL) was stirred at room temperature for 2 hours. The reaction mixture was concentrated, and the residue was washed with diethyl ether and dried to give the title compound (3.3 g, 86%) as a solid. LCMS-C: Retention Time 0.31 min; m/z 214.1 [M+H]⁺ for free base.

### (c) Methyl 4-((6-chloropyrimidin-4-yl)oxy)piperidine-1-carboxylate I84

To a solution of 4-chloro-6-(piperidin-4-yloxy)pyrimidine dihydrochloride I83 (3.3 g, 11.5 mmol) and Et₃N (5.8 g, 57.6 mmol) in DCM (40 mL) was added methyl chloroformate (1.3 g, 13.8 mmol). The reaction was stirred at room temperature for 2 hours. Water (100 mL) was added and the aqueous extracted with DCM (×2). The combined organic layers were washed with saturated aqueous NaHCO₃, water, and brine, dried (Na₂SO₄) and concentrated to give the title compound (2.6 g, 83%) as an oil. LCMS-C: Retention Time 2.47 min; m/z 272.1 [M+H]⁺.

### (d) Methyl 6-((1-(methoxycarbonyl)piperidin-4-yl)oxy)pyrimidine-4-carboxylate I85

A mixture of methyl 4-((6-chloropyrimidin-4-yl)oxy)piperidine-1-carboxylate I84 (500 mg, 1.84 mmol), PdCl₂(dppf) (67 mg, 0.09 mmol) and Et₃N (372 mg, 3.68 mmol) in MeOH (10 mL) under an atmosphere of CO(g) was heated at 50° C. for 6 hours. The reaction mixture was concentrated in vacuo and the residue obtained purified by silica gel chromatography (33% EtOAc in petroleum ether) to give the title compound (330 mg, 61%) as an oil. LCMS-C: Retention Time 2.35 min; m/z 296.3 [M+H]⁺.

### (e) Lithium 6-((1-(methoxycarbonyl)piperidin-4-yl)oxy)pyrimidine-4-carboxylate I86

To a solution of methyl 6-((1-(methoxycarbonyl)piperidin-4-yl)oxy)pyrimidine-4-carboxylate 185 (100 mg, 0.34 mmol) in a mixture of THF (1 mL), MeOH (2 mL) and water (1 mL) was added LiOH H₂O (30 mg, 0.68 mmol). The reaction was stirred at room temperature overnight, then concentrated, and the residue was lyophilized to give a crude product (110 mg) as solid which was used in the next step without further purification. LCMS-C: Retention Time 1.21 min; m/z 282.1 [M-Li+2H]⁺.

### (xxxiii) 2-Ethoxy-4-(2-oxo-2-(pyrrolidin-1-yl)ethyl)benzoic acid I88

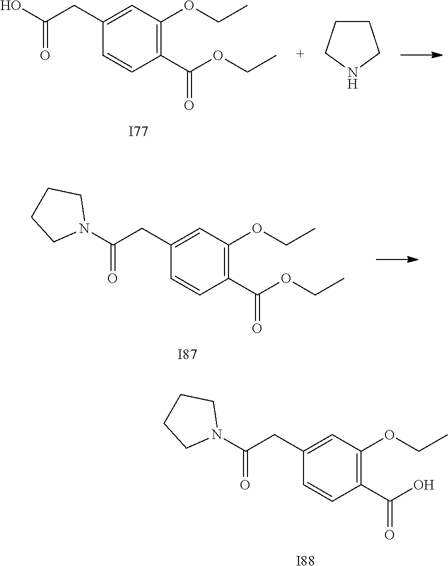

### (a) Ethyl 2-ethoxy-4-(2-oxo-2-(pyrrolidin-1-yl)ethyl)benzoate I87

A solution of 2-(3-ethoxy-4-(ethoxycarbonyl)phenyl)acetic acid (170 mg, 0.67 mmol), pyrrolidine (47.9 mg, 0.67 mmol), EDCI (154 mg, 0.8 mmol), HOBt (9.5 mg, 0.07 mmol) and DIPEA (173 mg, 1.34 mmol) in DCM (5 mL) was stirred at room temperature overnight. Water (5 mL) was added and the reaction mixture was extracted with DCM (3×5 mL). The combined organic layers were washed with saturated aqueous NaHCO₃, water and brine, dried (Na₂SO₄) and concentrated to give the title compound (170 mg, 83%) as an oil. LCMS-C: Retention Time 2.47 min; m/z 306.1 [M+H]⁺.

### 2-Ethoxy-4-(2-oxo-2-(pyrrolidin-1-yl)ethyl)benzoic acid I88

To a solution of ethyl 2-ethoxy-4-(2-oxo-2-(pyrrolidin-1-yl)ethyl)benzoate I87 (170 mg, 0.56 mmol) in a mixture of THF (2 mL), MeOH (4 mL) and water (2 mL) was added LiOH H₂O (47 mg, 1.1 mmol), and the mixture was stirred at room temperature overnight. The reaction mixture was concentrated in vacuo and the residue re-dissolved in water (5 mL) and acidified to pH 2 with 10% aqueous H₂SO₄. The aqueous layer was extracted with DCM (3×5 mL) and the combined organic fractions dried (Na₂SO₄) and concentrated to give the title compound (120 mg, 77%) as a solid. LCMS-C: Retention Time 1.76 min; m/z 278.1 [M+H]⁺.

### (xxxiv) 2-Ethoxy-3-methyl-4-(morpholine-4-carbonyl)benzoic acid I95

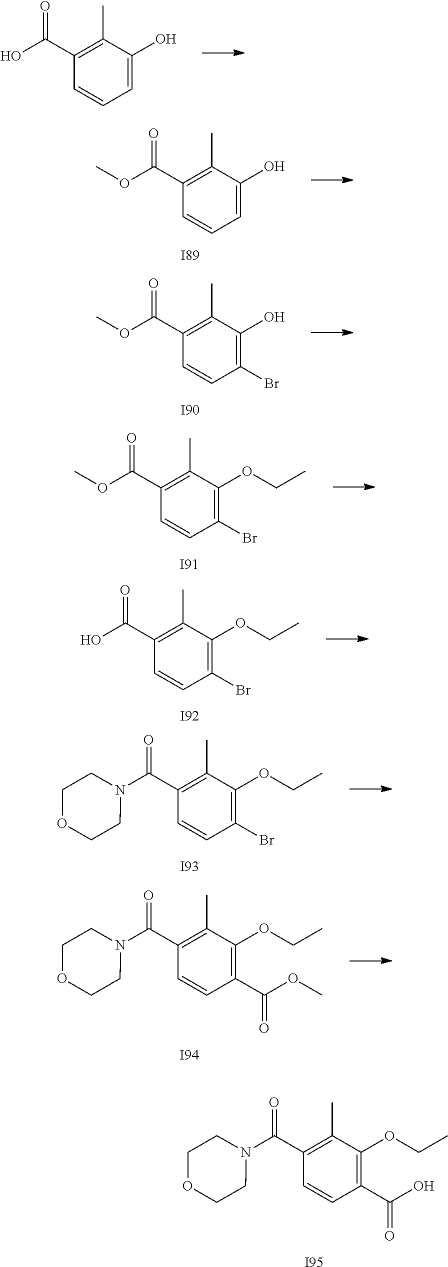

### (a) Methyl 3-hydroxy-2-methylbenzoate I89

To a solution of 3-hydroxy-2-methylbenzoic acid (10.0 g, 65.7 mmol) in MeOH (100 mL) was added SOCl₂ (15.6 g, 131.5 mmol) slowly and the mixture was stirred at 50° C. overnight. The reaction mixture was concentrated, and the residue dissolved in DCM (100 mL). The organic solution was washed with saturated aqueous NaHCO₃, dried (Na₂SO₄) and concentrated to give the title compound (10.7 g, 98%) as a solid. LCMS-C: Retention Time 1.98 min; m/z 167.1 [M+H]⁺.

### (b) Methyl 4-bromo-3-hydroxy-2-methylbenzoate I90

To a solution of tert-butylamine (2.0 g, 27.1 mmol) in DCM (180 mL) at −70° C. was added a solution of Br₂ (4.2 g, 27.1 mmol) in DCM (10 mL) dropwise and the mixture was stirred at −70° C. for 1 hour. A solution of methyl 3-hydroxy-2-methylbenzoate I89 (4.5 g, 27.1 mmol) in DCM (10 mL) was then added dropwise and the resulting mixture allowed to warm to room temperature and stirred overnight. The reaction was quenched by addition of water (30 mL) and the aqueous layer extracted with DCM (3×50 mL). The combined organic layers were washed with brine, dried (Na₂SO₄) and concentrated. The crude residue obtained was purified by silica gel chromatography (2% EtOAc in petroleum ether) to give the title compound (2.2 g, 33%) as a solid. LCMS-C: Retention Time 2.46 min; m/z 245.0, 247.0 [M+H]⁺. ¹H NMR (400 MHz, DMSO-d₆) δ 9.38 (s, 1H), 7.46 (d, J=8.4 Hz, 1H), 7.17 (d, J=8.4 Hz, 1H), 3.80 (s, 3H), 2.38 (s, 3H).

### (c) Methyl 4-bromo-3-ethoxy-2-methylbenzoate I91

To a solution of methyl 4-bromo-3-hydroxy-2-methylbenzoate I90 (2.1 g, 8.6 mmol) in DMSO (10 mL) was added K₂CO₃ (3.5 g, 25.8 mmol) and ethyl bromide (1.4 g, 12.9 mmol). The reaction was stirred at 40° C. overnight. Water (30 mL) was added and the aqueous extracted with DCM (3×20 mL). The organic layer was washed with water (10×30 mL), dried (Na₂SO₄), and concentrated to give the title compound (2.2 g, 95%) as a solid. LCMS-C: Retention Time 3.08 min; m/z 273.0, 275.0 [M+H]⁺.

### (d) 4-Bromo-3-ethoxy-2-methylbenzoic acid I92

To a solution of methyl 4-bromo-3-ethoxy-2-methylbenzoate I91 (2.1 g, 10.0 mmol) in a mixture of MeOH (10 mL) and water (0.2 mL) was added NaOH (0.4 g, 20.0 mmol). The reaction was stirred at room temperature overnight then the solvent was removed under reduced pressure. The residue obtained was re-dissolved in water (20 mL) and acidified with 1M HCl to pH 2. The aqueous layer was extracted with DCM (4×20 mL) and the combined organic layers dried (Na₂SO₄) and concentrated to give the title compound (2.4 g, 92%) as a solid. LCMS-C: Retention Time 2.75 min; m/z 281.0, 283.0 [M+Na]⁺.

### (e) (4-Bromo-3-ethoxy-2-methylphenyl)(morpholino)methanone I93

To a solution of 4-bromo-3-ethoxy-2-methylbenzoic acid I92 (2.4 g, 9.3 mmol) in DCM (500 mL) at 0° C. was added oxalyl chloride (3.5 g, 27.9 mmol) and DMF (0.2 mL). The reaction was stirred for 3 hours then morpholine (1.6 g, 18.6 mmol) and triethylamine (4.1 g, 40.9 mmol) were added and stirring was continued overnight at 0° C. Water (100 mL) was added and the mixture was extracted with DCM (2×100 mL), the combined organic layers were washed with brine, dried (Na₂SO₄) and concentrated. The residue obtained was purified by silica gel chromatography (1% MeOH in DCM) to give the title compound (720 mg, 24%) as a solid. LCMS-C: Retention Time 2.48 min; m/z 328.1, 330.1 [M+H]⁺.

### (f) Methyl 2-ethoxy-3-methyl-4-(morpholine-4-carbonyl)benzoate I94

To a solution of (4-bromo-3-ethoxy-2-methylphenyl)(morpholino)methanone I93 (720 mg, 2.2 mmol) in MeOH (10 mL) was added PdCl₂(dppf) (81 mg, 0.11 mmol) and TEA (489 mg, 4.8 mmol). The reaction was heated at reflux overnight under a CO atmosphere. The mixture was concentrated, and the residue obtained dissolved in DCM (20 mL), washed with water and brine, dried (Na₂SO₄) and concentrated. The residue obtained was purified by silica gel chromatography (1% MeOH in DCM) to give the title compound (670 mg) as an oil. LCMS-C: Retention Time 2.02 min; m/z 308.2 [M+H]⁺.

### (g) 2-Ethoxy-3-methyl-4-(morpholine-4-carbonyl)benzoic acid I95

To a solution of methyl 2-ethoxy-3-methyl-4-(morpholine-4-carbonyl)benzoate I94 (670 mg, 2.2 mmol) in a mixture of THF (1 mL), MeOH (10 mL) and water (0.1 mL) was added LiOH H₂O (275 mg, 6.5 mmol) The reaction was stirred at room temperature overnight, then concentrated under reduced pressure. The residue obtained was re-dissolved in water (20 mL) then acidified with 1M HCl to pH 2. The aqueous mixture was extracted with DCM (10 mL×3) and the combined organic layers were dried (Na₂SO₄) and concentrated to give the title compound (310 mg, 48%) as an oil. LCMS-C: Retention Time 0.89 min; m/z 294.1 [M+H]⁺.

### (xxxv) 2-Fluoro-4-(3-oxa-8-aza-bicyclo[3.2.1]octane-8-carbonyl)-benzoic acid I97

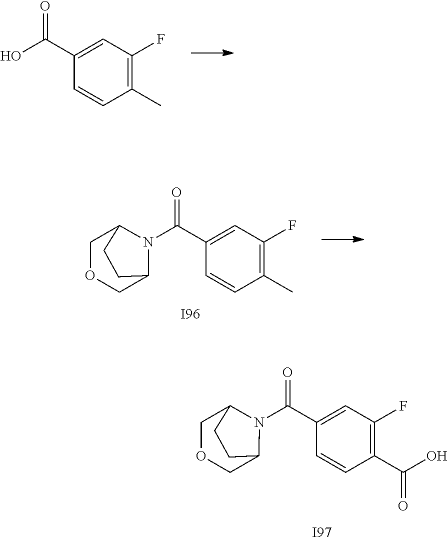 [SMILES: Cc1ccc(C(=O)O)cc1F ; "I96" ; Cc1ccc(C(=O)N2C3CCC2COC3)cc1F ; "I97" ; O=C(O)c1ccc(C(=O)N2C3CCC2COC3)cc1F]

### (a) (3-Fluoro-4-methyl-phenyl)-(3-oxa-8-aza-bicyclo[3.2.1]oct-8-yl)-methanone I96

To a solution of 3-fluoro-4-methylbenzoic acid (2.0 g, 13.0 mmol) and 3-oxa-8-azabicyclo[3.2.1]octane hydrochloride (1.8 g, 11.8 mmol) in DCM (10 mL) were added DIPEA (6.3 mL, 35.4 mmol), EDCI (2.72 g, 14.2 mmol) and HOBt (162.1 g, 1.2 mmol). The mixture was then stirred at room temperature overnight. Saturated aqueous NaHCO₃ (50 mL) was added and the mixture was extracted with DCM (3×50 mL). The combined organic extracts were dried (Na₂SO₄) and concentrated, and the residue was purified by chromatography (5% EtOAc/petroleum ether) to give the title compound as a solid (1.7 g, 58%). LCMS-C: Retention Time 2.30 min; m/z 250.1 [M+H]⁺.

### (b) 2-Fluoro-4-(3-oxa-8-aza-bicyclo[3.2.1]octane-8-carbonyl)-benzoic acid I97

To a solution of (3-fluoro-4-methyl-phenyl)-(3-oxa-8-aza-bicyclo[3.2.1]oct-8-yl)-methanone I96 (800 mg, 3.2 mmol) in a mixture of pyridine (6 mL) and H₂O (12 mL) was added KMnO₄ (5.1 g, 32 mmol), and the mixture was stirred at room temperature for 2 days. The resulting suspension was filtered through Celite and the filtrate washed with DCM (3×50 mL). The remaining aqueous layer was acidified to pH 3 by addition of 2M HCl, and extracted with DCM (4×60 mL). The combined organic layers were dried (Na₂SO₄) and concentrated to give the title compound as a solid (500 mg, 56%). LCMS-C: Retention Time 2.89 min; m/z 280.1 [M+H]⁺.

### (xxxvi) 6-(3-Oxa-8-azabicyclo[3.2.1]octane-8-carbonyl)-4-ethoxynicotinic acid I103

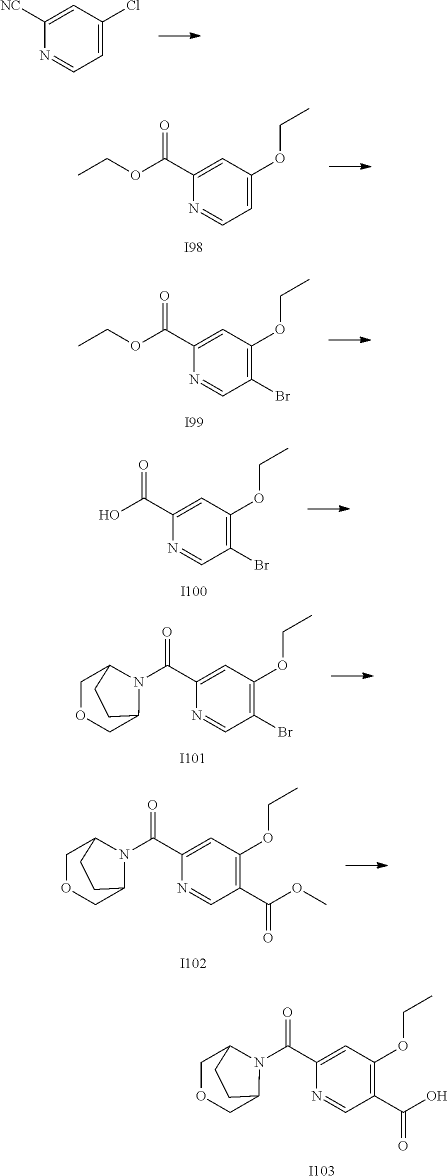

### (a) Ethyl 4-ethoxypicolinate I98

A solution of 4-chloropicolinonitrile (5.5 g, 39.7 mmol) in saturated HCl/EtOH solution (80 mL) was stirred at 80° C. for 2 days. The solvent was removed under reduced pressure, saturated aqueous NaHCO₃ (200 mL) was added and the aqueous layer extracted with DCM (3×200 mL). The pooled organic extracts were dried (Na₂SO₄) and concentrated to give the title compound as a solid (2.6 g, 34%). ¹H NMR (400 MHz, CDCl₃) δ 8.52 (d, J=5.6 Hz, 1H), 7.63 (d, J=2.4 Hz, 1H), 6.93-6.91 (m, 1H), 4.80-4.43 (m, 2H), 4.16-4.11 (m, 2H), 1.46-1.41 (m, 6H). LCMS-C: Retention Time 1.35 min; m/z 196.1[M+H]⁺.

### (b) Ethyl 5-bromo-4-ethoxypicolinate I99

To a solution of ethyl 4-ethoxypicolinate I98 (1.6 g, 8.2 mmol) in concentrated H₂SO₄ (80 mL) was added NBS (2.7 g, 14.8 mmol). The reaction was stirred at room temperature overnight then quenched by addition of saturated aqueous NaHCO₃ (150 mL). The aqueous layer was extracted with DCM (3×130 mL) and the combined organic layers dried (Na₂SO₄) and concentrated to give the title compound as a solid (2.0 g, 91%). ¹H NMR (400 MHz, CDCl₃) δ 8.67 (s, 1H), 7.63 (s, 1H), 4.94-4.40 (m, 2H), 4.29-4.23 (m, 2H), 1.54-1.51 (m, 3H), 1.46-1.42 (m, 3H); LCMS-C: Retention Time 2.59 min; m/z 274.0, 276.0 [M+H]⁺.

### (c) 5-Bromo-4-ethoxypicolinic acid I100

To a solution of ethyl 5-bromo-4-ethoxypicolinate I99 (1.6 g, 6.0 mmol) in a mixture of THF (20 mL), MeOH (2 mL) and H₂O (0.2 mL) was added LiOH H₂O (1.0 g, 24.0 mmol). The reaction was stirred at room temperature for 2 days. The solvent was removed under reduced pressure and the residue obtained dissolved in water (10 mL) and acidified to pH 3 with 1M HCl. The aqueous phase was extracted with DCM (4×50 mL) and the pooled organic extracts dried (Na₂SO₄) and concentrated to give the title compound as a solid (800 mg, 54%). LCMS-C: Retention Time 0.86 min; m/z 246.0, 248.0 [M+H]⁺

### (d) 3-Oxa-8-azabicyclo[3.2.1]octan-8-yl(5-bromo-4-ethoxypyridin-2-yl)methanone I101

To a solution of 5-bromo-4-ethoxypicolinic acid I100 (300 mg, 1.2 mmol) and 3-oxa-8-azabicyclo[3.2.1]octane hydrochloride (153 mg, 1.0 mmol) in DCM (5 mL) were added DIPEA (0.72 mL, 4.1 mmol), EDCI (393 mg, 2.0 mmol) and HOBt (15 mg, 0.1 mmol). The mixture was stirred at room temperature overnight, then quenched by addition of saturated aqueous NaHCO₃ (50 mL) and the mixture was extracted with DCM (3×50 mL). The combined organic layers were dried (Na₂SO₄) and concentrated, and the residue obtained purified by chromatography (20% EtOAc/petroleum ether) to give the title compound as a solid (262 mg, 75%). LCMS-C: Retention Time 5.56 min; m/z 340.9, 342.9 [M+H]⁺

### (e) Methyl 6-(3-oxa-8-azabicyclo[3.2.1]octane-8-carbonyl)-4-ethoxynicotinate I102

To a solution of 3-oxa-8-azabicyclo[3.2.1]octan-8-yl(5-bromo-4-ethoxypyridin-2-yl)methanone 1101 (650 mg, 1.9 mmol) in MeOH (50 mL) was added Et₃N (577 mg, 5.7 mmol) and PdCl₂(dppf) (73 mg, 0.05 mmol). The reaction was heated at reflux for 2 days under an atmosphere of CO. The solvent was removed under reduced pressure and the residue obtained taken up in water (100 mL) and extracted with DCM (3×100 mL). The combined organic layers were dried over (Na₂SO₄) and concentrated. The residue obtained was purified by chromatography (33% EtOAc/petroleum ether) to give the title compound as a solid (400 mg, 66%). LCMS-C: Retention Time 4.94 min; m/z 320.8 [M+H]⁺.

### (f) 6-(3-Oxa-8-azabicyclo[3.2.1]octane-8-carbonyl)-4-ethoxynicotinic acid I103

To a solution of methyl 6-(3-oxa-8-azabicyclo[3.2.1]octane-8-carbonyl)-4-ethoxynicotinate I102 (350 mg, 1.1 mmol) in MeOH (20 mL) was added 1 M aqueous NaOH (2.2 mL) and the mixture was stirred at room temperature overnight. The solvent was removed under reduced pressure and the residue obtained taken up in water (15 mL). The aqueous was acidified to pH 4 by addition of 1M HCl, then extracted with DCM (4×60 mL) and the pooled extracts dried (Na₂SO₄) and concentrated to give the title compound as a solid (320 mg, 95%). LCMS-C: Retention Time 0.61 min; m/z 307.1 [M+H]⁺.

### (xxxvii) 4-(3-Oxa-8-azabicyclo[3.2.1]octane-8-carbonyl)-2-chlorobenzoic acid 105

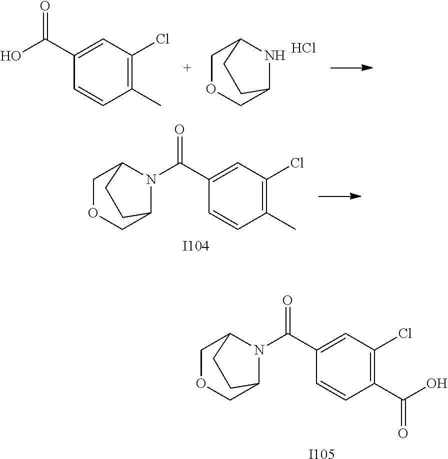

### 3-Oxa-8-azabicyclo[3.2.1]octan-8-yl(3-chloro-4-methylphenyl)methanone I104

To a solution of 3-chloro-4-methylbenzoic acid (2.26 g, 13.24 mmol) in DCM (20 mL) was added DIPEA (4.7 g, 36.10 mmol), EDCI (3.5 g, 18.05 mmol), HOBt (190 mg, 1.40 mmol) and 3-oxa-8-azabicyclo[3.2.1]octane hydrochloride (1.8 g, 12.03 mmol). The resulting mixture was stirred at room temperature overnight, then diluted with water (10 mL) and partitioned against DCM (10 mL). The aqueous layer was extracted with DCM (3×5 mL) and the combined organic extracts washed with saturated NaHCO₃ (3×5 mL) and brine (3×5 mL), dried (Na₂SO₄), filtered and concentrated. The crude product was purified by silica gel chromatography (ethyl acetate/petroleum ether=1/10) to give the title compound (3.0 g, 92%) as a solid. ¹HNMR (400 MHz, Methanol-d₄) δ 7.50 (d, J=1.6 Hz, 1H), 7.40 (d, J=7.6 Hz, 1H), 7.33 (dd, J=7.6, 1.6 Hz, 1H), 4.61-4.56 (m, 1H), 3.99 (s, 1H), 3.78-3.59 (m, 4H), 2.41 (s, 3H), 2.00-1.99 (m, 4H); LCMS-C: Retention Time 2.51 min; m/z 266.1 [M+H]⁺

### (b) 4-(3-oxa-8-azabicyclo[3.2.]octane-8-carbonyl)-2-chlorobenzoic acid I105

To a solution of 3-oxa-8-azabicyclo[3.2.1]octan-8-yl(3-chloro-4-methylphenyl)methanone I104 (500 mg, 1.88 mmol) in a mixture of pyridine (5 mL) and water (15 mL) was added KMnO₄ (1.78 g, 11.28 mmol) in portions. The resulting mixture was heated to 50° C. and stirred for 48 hours. The mixture was filtered and the filtrate was extracted with ethyl acetate (3×5 mL). The pH of the aqueous phase was adjusted to pH 1-2 by addition of concentrated HCl, then extracted with DCM (8×5 mL). The combined organic extracts were dried (Na₂SO₄), filtered and concentrated to give the title compound (340 mg, 61%) as a solid. LCMS Retention Time 0.88 min; m/z 296.1 [M+H]⁺.

### (xxxviii) 2-(Trifluoromethyl)-1H-benzo[d]imidazole-6-carboxylic acid I106

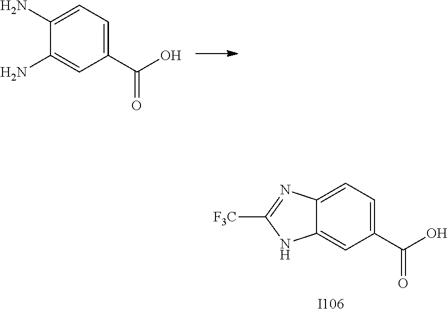 [SMILES: Nc1ccc(C(=O)O)cc1N ; "I106" ; O=C(O)c1ccc2nc(C(F)(F)F)[nH]c2c1]

A solution of 3,4-diaminobenzoic acid (1.0 g, 6.6 mmol) in TFA (15 mL) was stirred at 70° C. for 16 hours. The reaction mixture was concentrated, the crude material was purified by column chromatography (DCM:methanol=50:1) to give the title compound as a solid (1.3 g, 86%). LCMS-C: Retention Time 1.76 min; m/z 231.1 [M+H]⁺.

### (xxxix) 4-(3-Oxa-8-azabicyclo[3.2.1]octane-8-carbonyl)-3-(difluoromethyl)benzoic acid I110

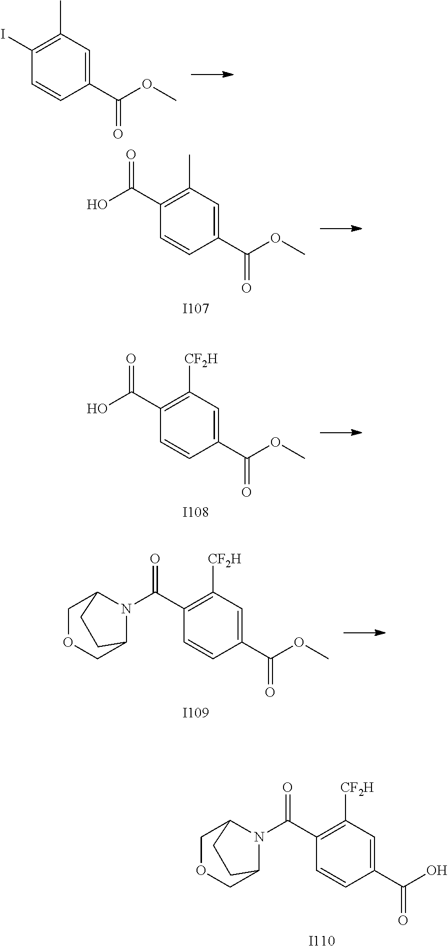

### (a) 4-(Methoxycarbonyl)-2-methylbenzoic acid I107

A solution of methyl 4-iodo-3-methylbenzoate (2.0 g, 7.2 mmol) in THF (30 mL) under N₂ was cooled to −20° C., a solution of iso-propylmagnesium chloride (2 M in THF, 4 mL, 8.0 mmol) was added dropwise and the suspension stirred at −20° C. for 1 hour. CO₂ (g) was bubbled through the mixture, and the reaction stirred at room temperature for 1 hour. The solvent was evaporated and water was added, the aqueous layer was washed with DCM (15 mL×3) and the pH adjusted to 3 by addition of 3M HCl. The mixture was extracted with DCM (15 mL×3) and the combined organic layers dried (Na₂SO₄) and concentrated to give the title compound as a solid (870 mg, 88%). LCMS-C: Retention Time 2.26 min; m/z 195.1 [M+H]⁺.

### (b) 2-(Difluoromethyl)-4-(methoxycarbonyl)benzoic acid I108

To a solution of 4-(methoxycarbonyl)-2-methylbenzoic acid I107 (450 mg, 2.3 mmol) in MeCN (20 mL) and water (20 mL) was added AgNO₃ (79 mg, 0.5 mmol), Na₂S₂O₈ (552 mg, 2.3 mmol) and Selectfluor® (4.6 g, 13.9 mmol). The resulting mixture was stirred at 80° C. for 6 hours. Further AgNO₃ (19 mg, 0.003 mmol), Na₂S₂O₈ (138 mg, 0.6 mmol) and Selectfluor® (1.2 g, 3.5 mmol) were added and the resulting mixture was stirred at 80° C. for 3 hours. The pH of the reaction mixture was adjusted to pH 9 by addition of saturated aqueous NaHCO₃ solution and washed with DCM (20 mL×2). The pH of the aqueous fraction that remained was adjusted to 3-4 by addition of 2M HCl solution and extracted with DCM (20 mL×3). The combined organic layers were washed with brine (10 mL×2), dried (Na₂SO₄) and concentrated to give the title compound (180 mg, 41%) as a solid. LCMS-C: Retention Time 2.30 min; m/z 229.1 [M−H]⁺.

### (c) Methyl 4-(3-oxa-8-azabicyclo[3.2.1]octane-8-carbonyl)-3-(difluoromethyl)benzoate I109

To a solution of 3-oxa-8-azabicyclo[3.2.1]octane hydrochloride (59 mg, 0.4 mmol) in DCM (5 mL) was added 2-(difluoromethyl)-4-(methoxycarbonyl)benzoic acid I108 (100 mg, 0.4 mmol), HOBt (5.3 mg, 0.04 mmol), DIPEA (102 mg, 0.79 mmol) and EDCI (91 mg, 0.5 mmol). The resulting mixture was stirred at room temperature overnight. The reaction mixture was partitioned against saturated aqueous NaHCO₃, the organic layer was washed with brine (4 mL×2), dried (Na₂SO₄) and concentrated. The residue was purified by prep TLC (DCM: methanol=20:1) to give the title compound as a solid (45 mg, 35%). LCMS-C: Retention Time 2.40 min; m/z 326.1 [M+H]⁺.

### (d) 4-(3-Oxa-8-azabicyclo[3.2.1]octane-8-carbonyl)-3-(difluoromethyl)benzoic acid I110

To a solution of methyl 4-(3-oxa-8-azabicyclo[3.2.1]octane-8-carbonyl)-3-(difluoromethyl) benzoate I109 (42 mg, 0.13 mmol) in a mixture of THF (5 mL), methanol (0.5 mL) and water (0.5 mL) was added LiOH H₂O (27 mg, 0.65 mmol). The resulting mixture was stirred at room temperature overnight. The reaction mixture was filtered and concentrated under reduced pressure. The residue obtained was diluted with water (3 mL) and the pH of the aqueous mixture was adjusted to 6 by addition of 2 M HCl. The aqueous solution was extracted with DCM (3 mL×2) and the combined organic layers washed with brine (2 mL×2), dried (Na₂SO₄) and concentrated to give the title compound as a solid (49 mg, quantitative yield). LCMS-C: Retention Time 1.95 min; m/z 312.1 [M+H]⁺.

### (xl) 4-(3-Oxa-8-azabicyclo[3.2.1]octane-8-carbonyl)-2-ethoxybenzoic acid I112

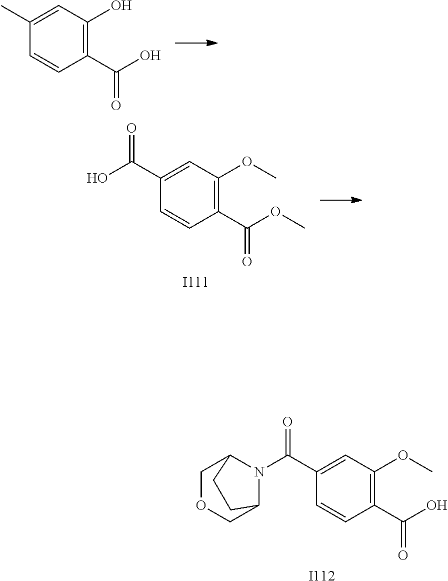

### (a) 3-Methoxy-4-(methoxycarbonyl)benzoic acid I111

To a mixture of 2-hydroxy-4-methylbenzoic acid (10.0 g, 65.7 mmol) and K₂CO₃ (22.7 g, 164.3 mmol) in DMF (200 mL) at room temperature was added methyl iodide (20.5 g, 144.5 mmol) over a period of 10 minutes. The resulting mixture was stirred at room temperature overnight, then diluted with DCM (150 mL) and filtered. The filtrate was washed with water (200 mL×10) and brine (200 mL×2), dried (Na₂SO₄) and concentrated to give the intermediate as a liquid (11.0 g).

To a solution of the intermediate in a mixture of pyridine (30 mL) and water (90 mL) was added KMnO₄ (30.37 g, 192.2 mmol). The resulting mixture was heated at 50° C. for 48 hours, then cooled and allowed to stir at room temperature for 24 hours. The mixture was filtered and the filter cake washed with hot water. The combined aqueous filtrates were washed with EtOAc (75 mL×3) and acidified to pH 2 with 2M aqueous HCl solution. The mixture was extracted with DCM (150 mL×3) and the combined organic layers were washed with brine, dried (Na₂SO₄) and concentrated to give the title compound as a solid (7.0 g, 51% yield over 2 steps): LCMS-C: Retention Time 1.24 min; m/z 211.0 [M+H]⁺.

### (b) 4-(3-Oxa-8-azabicyclo[3.2.1]octane-8-carbonyl)-2-methoxybenzoic acid I112

To a solution of 3-methoxy-4-(methoxycarbonyl)benzoic acid I111 (0.5 g, 2.4 mmol) in DCM (20 mL) was added 3-oxa-8-azabicyclo[3.2.1]octane hydrochloride (0.38 g, 2.5 mmol), HOBt (0.43 g, 3.2 mmol), triethylamine (0.85 g, 8.4 mmol) and EDCI (0.60 g, 3.2 mmol). The resulting mixture was stirred at room temperature overnight. Saturated aqueous NaHCO₃ was added, the organic layer was separated and the aqueous extracted with DCM (20 mL×2). The combined organic layers were washed with brine, dried (Na₂SO₄) and concentrated. The residue was purified by column chromatography (5% methanol in dichloromethane) to give the intermediate as a solid.

To a solution of the intermediate in a mixture of THF (4 mL) and methanol (4 mL) was added aqueous NaOH (2 M, 4 mL). The resulting mixture was then stirred at room temperature for 14 hours. The solvent was removed, and the residue obtained was diluted with water (20 mL). The pH of the aqueous mixture was adjusted to 6 by addition of 2 M aqueous HCl solution. The mixture was extracted with DCM (20 mL×3) and the combined organic layers washed with brine (10 mL×2), dried (Na₂SO₄) and concentrated to give the title compound as a solid (0.7 g, quantitative yield over 2 steps). LCMS-C: Retention Time 0.55 min; m/z 292.1 [M+H]⁺.

### (xli) 2-(1-Acetylpiperidin-4-yl)-1H-benzo[d]imidazole-5-carboxylic acid I117

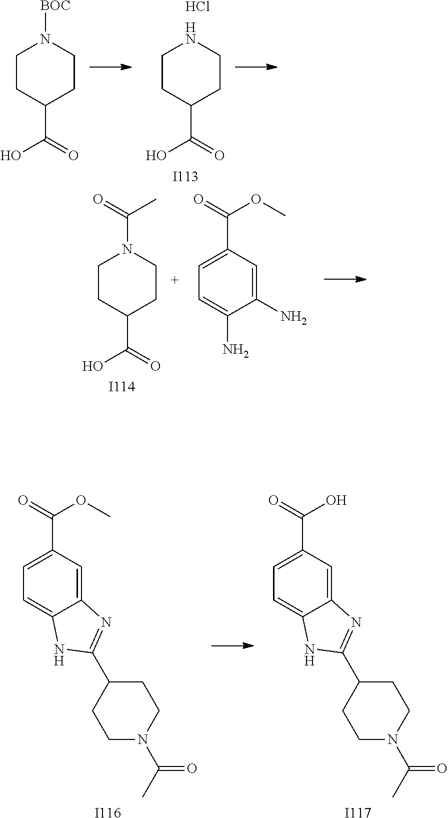

### (a) Piperidine-4-carboxylic acid hydrochloride I113

A mixture of 1-(tert-butoxycarbonyl)piperidine-4-carboxylic acid (1.0 g, 4.4 mmol) in saturated EtOAc/HCl solution (10 mL) was stirred at room temperature for 2 hours. The mixture was concentrated to give the title compound as a solid. LCMS-C: Retention Time 0.72 min; m/z 130.1 [M+H]⁺ for free base.

### (b) 1-Acetylpiperidine-4-carboxylic acid I114

To a mixture of piperidine-4-carboxylic acid hydrochloride I113 (720 mg, 4.3 mmol) and Et₃N (528 mg, 5.2 mmol) in DCM (10 mL) was added Ac₂O (488 mg, 4.8 mmol). The reaction was stirred at room temperature overnight then concentrated. The residue obtained was recrystallized from EtOH to give the title compound as a solid. LCMS-C: Retention Time 0.73 min; m/z 172.1 [M+H]⁺.

### (c) Methyl 2-(1-acetylpiperidin-4-yl)-1H-benzo[d]imidazole-5-carboxylate I116

A mixture of 1-acetylpiperidine-4-carboxylic acid I114 (200 mg, 1.2 mmol), methyl 3,4-diaminobenzoate (I94 mg, 1.2 mmol), EDCI (269 mg, 1.4 mmol), HOBt (16 mg, 0.12 mmol) and DIPEA (302 mg, 2.3 mmol) in DCM (5 mL) was stirred at room temperature overnight. The precipitate was collected by filtration, and the filter cake dried to give an intermediate compound (182 mg, 49%) as an oil. LCMS-C: Retention Time 4.27 min; m/z 319.9 [M+H]⁺.

A mixture of the intermediate compound (130 mg, 0.41 mmol) in AcOH (5 mL) was heated at 65° C. for 3 hours to form a clear solution. The reaction mixture was neutralised with saturated aqueous NaHCO₃ to pH 6-7 and extracted with DCM (3×10 mL). The combined organic layers were washed with brine and dried (Na₂SO₄). The chemistry was repeated with a further 50 mg of starting material, and the product from the two batches combined to give the title compound (110 mg, 65%). LCMS-C: Retention Time 4.28 min; m/z 301.9 [M+H]⁺.

### (d) 2-(1-Acetylpiperidin-4-yl)-1H-benzo[d]imidazole-5-carboxylic acid I117

To a mixture of methyl 2-(1-acetylpiperidin-4-yl)-1H-benzo[d]imidazole-5-carboxylate I116 (110 mg, 0.36 mmol) in a mixture of THF (1 mL), MeOH (2 mL) and water (1 mL) was added LiOH H₂O (136 mg, 3.2 mmol), and the mixture was stirred at room temperature overnight. The reaction mixture was concentrated, and the residue obtained was re-dissolved in water (3 mL) then neutralised by addition of 10% aqueous H₂SO₄ to pH 6. The aqueous solution was lyophilized to give the crude title compound (300 mg) as solid containing some Li₂SO₄ which was used in the next step without purification. LCMS-C: Retention Time 0.30 min; m/z 288.1 [M+H]⁺.

### (xlii) 2-Ethoxy-4-(5-methyl-1,3,4-oxadiazol-2-yl)benzoic acid I120

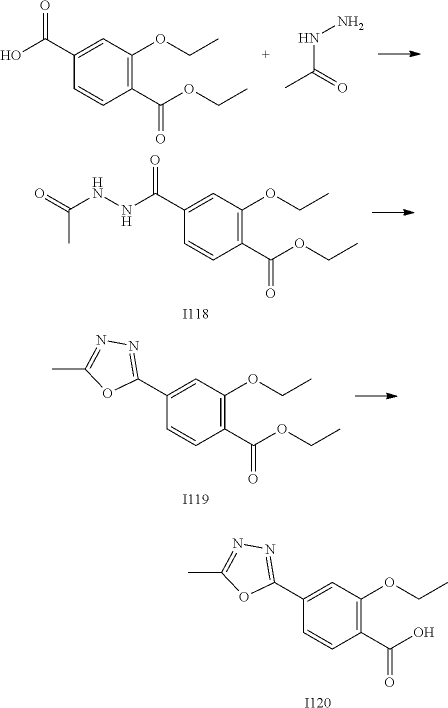

### (a) Ethyl 4-(2-acetylhydrazinecarbonyl)-2-ethoxybenzoate I118

To a solution of 3-ethoxy-4-(ethoxycarbonyl)benzoic acid I40 (500 mg, 2.1 mmol) in DCM (20 mL) was added DIPEA (960 mg, 7.4 mmol), HOBt (30 mg, 0.2 mmol), EDCI (800 mg, 4.2 mmol), and acetohydrazide (156 mg, 2.1 mmol). The mixture was stirred at room temperature overnight then diluted with DCM (50 mL). The organic layer was washed with water (50 mL) and brine (50 mL), dried (Na₂SO₄) and concentrated. The residue was purified by column chromatography (100% DCM to 4% MeOH in DCM) to give the title compound as a solid (350 mg, 57%). LCMS-C: Retention Time 4.79 min; m/z 294.9 [M+H]⁺.

### (b) Ethyl 2-ethoxy-4-(5-methyl-1,3,4-oxadiazol-2-yl)benzoate I119

A mixture of ethyl 4-(2-acetylhydrazinecarbonyl)-2-ethoxybenzoate I118 (330 mg, 1.1 mmol) in POCl₃ (3 mL) was heated at reflux for 1.5 hours. The mixture was poured into ice-water (20 mL), and the aqueous layer extracted with DCM (20 mL×2). The combined organic layers were washed with saturated NaHCO₃ (40 mL×3) and brine (40 mL), dried (Na₂SO₄) and concentrated to give the crude product as a solid (290 mg, 94%). The crude product was used for the next step without purification. LCMS-C: Retention Time 5.40 min; m/z 277.0 [M+H]⁺.

### (c) 2-Ethoxy-4-(5-methyl-1,3,4-oxadiazol-2-yl)benzoic acid I120

To a solution of ethyl 2-ethoxy-4-(5-methyl-1,3,4-oxadiazol-2-yl)benzoate I119 (270 mg, 1.0 mmol) in MeOH (10 mL) was added a solution of NaOH (193 mg, 4.8 mmol) in water (2 mL). The resulting mixture was stirred at room temperature overnight. The solvent was removed, and the residue obtained suspended in water (5 mL). The pH of the aqueous solution was adjusted to pH 4-5 by addition of 1 M aqueous HCl solution. The solid which precipitated was collected by filtration, washed with water (5 mL) and dried to give the title compound as a solid (110 mg, 45%). LCMS-C: Retention Time 4.77 min, m/z 249.0 [M+H]⁺

### (xliii) 2-Chloro-6-(oxetan-3-yloxy)isonicotinic acid I121

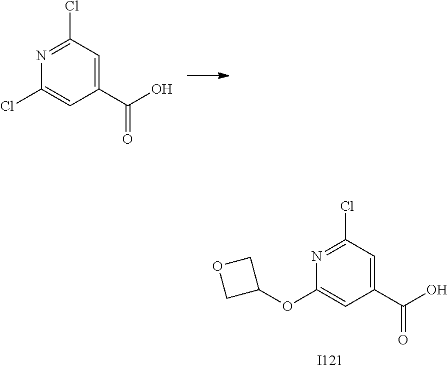 [SMILES: O=C(O)c1cc(Cl)nc(Cl)c1 ; "I121" ; O=C(O)c1cc(Cl)nc(OC2COC2)c1]

To a mixture of oxetan-3-ol (1.1 g 15.6 mmol) in dry DMF (20 mL) was added NaH (60% in mineral oil, 832 mg, 20.8 mmol) at 0° C. under nitrogen atmosphere and the mixture was stirred for 30 minutes, then 2,6-dichloroisonicotinic acid (2.0 g, 10.4 mmol) was added. The resulting mixture was heated to 50° C. overnight. Water (20 mL) was added and the mixture was acidified with 1 M HCl to form a suspension. The solid was collected by filtration to give the title compound (2.0 g, 83%) as a solid. LCMS-C: Retention Time 2.24 min; m/z 230.0, 232.0 [M+H]⁺

### (xliv) 6-((1-Acetylpiperidin-4-yl)oxy)pyridazine-4-carboxylic acid I124

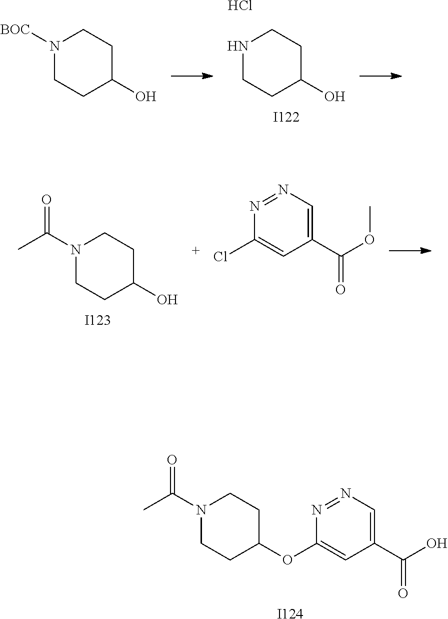

### (a) Piperidin-4-ol hydrochloride I122

To a solution of tert-butyl 4-hydroxypiperidine-1-carboxylate (10.9 g, 49.7 mmol, 1.0 eq) in EtOAc (40 mL) was added a saturated solution of HCl in EtOAc (10 mL), and the resulting mixture was stirred at room temperature overnight. The mixture was concentrated to give the title compound (7.0 g) as a solid.

### (b) I-(4-hydroxypiperidin-1-yl) ethanone I123

A mixture of piperidin-4-ol hydrochloride salt I122 (1.63 g, 11.9 mmol), acetic anhydride (1.82 g, 17.8 mmol), and K₂CO₃ (4.11 g, 29.7 mmol) in acetone (25 mL) was stirred at room temperature overnight. The reaction mixture was filtered and the filtrate was concentrated to give the crude title compound (1.6 g) as oil which was used for the next step without further purification. LCMS-C: Retention Time 0.29 min; m/z 144.1 [M+H]+

### (c) 6-((1-Acetylpiperidin-4-yl)oxy)pyridazine-4-carboxylic acid I124

To a solution of 1-(4-hydroxypiperidin-1-yl)ethanone I123 (166 mg, 1.16 mmol, 2.0 eq) in DMF (5 mL) at 0° C. was added NaH (60% in mineral oil, 58 mg, 1.45 mmol, 2.5 eq). Methyl 6-chloropyridazine-4-carboxylate (100 mg, 0.58 mmol, 1.0 eq) was added and the reaction was allowed to warm slowly to room temperature and stirred overnight. The reaction was quenched by addition of saturated aqueous NaHCO₃ (15 mL) and the aqueous layer was washed with DCM (15 mL×3). The aqueous layer was acidified to pH 5 with 1M HCl and extracted with DCM (15 mL×3), the combined organic fractions were dried (MgSO₄) and concentrated to give the crude title compound (130 mg) as an oil. LCMS-C: Retention Time 2.19 min, m/z 266.1 [M+H]⁺

### (xlv) 1-(Tetrahydro-2H-pyran-4-yl)-1H-benzo[d]imidazole-4-carboxylic acid I129

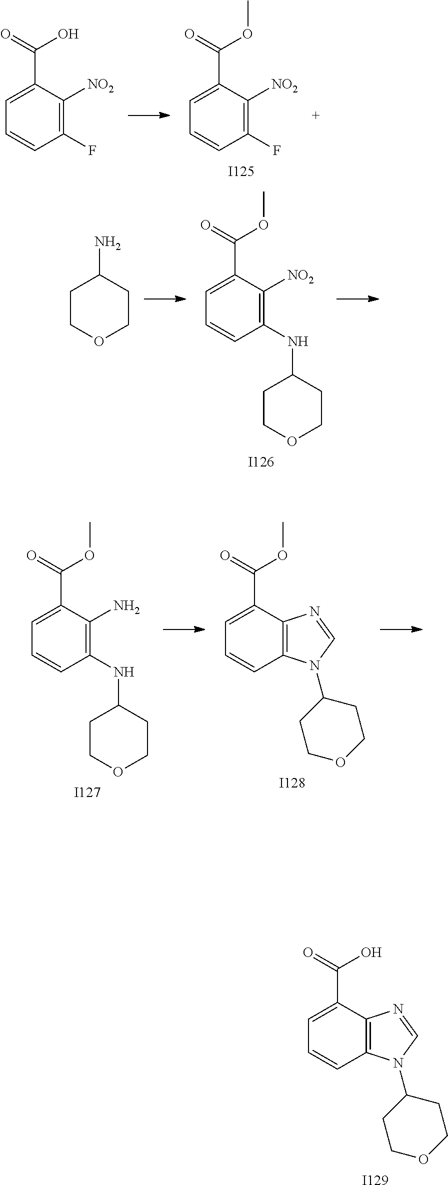

### (a) Methyl 3-fluoro-2-nitrobenzoate I125

To a solution of 3-fluoro-2-nitrobenzoic acid (1.0 g, 5.4 mmol) in DCM (15 mL) and DMF (1 drop) was added (COCl)₂ (1.4 g, 10.8 mmol) dropwise. The mixture was stirred at room temperature for 2 hours. Methanol (5 mL) was added dropwise to the mixture and stirring was continued for 1 hour. The solvent was removed, and the residue was dissolved in DCM (50 mL), the organic layer was washed with saturated NaHCO₃ (50 mL), water (50 mL) and brine (50 mL), dried (Na₂SO₄) and concentrated to give the title compound as a solid (1.0 g, 93%). LCMS-C: Retention Time 2.30 min; m/z 222.0 [M+Na]⁺.

### (b) Methyl 2-nitro-3-((tetrahydro-2H-pyran-4-yl)amino)benzoate I126

To a solution of methyl 3-fluoro-2-nitrobenzoate I125 (100 mg, 0.5 mmol) in MeCN (5 mL) were added tetrahydro-2H-pyran-4-amine (50 mg, 0.5 mmol) and DIPEA (97 mg, 0.75 mmol). The mixture was heated at 50° C. overnight. The solvent was removed, and the residue diluted with EtOAc (30 mL). The organic layer was washed with water (30 mL×2) and brine (30 mL), dried (Na₂SO₄) and concentrated to give the title compound as a solid (130 mg, 93%). LCMS-C: Retention Time 2.28 min; m/z 281.1 [M+H]⁺.

### (c) Methyl 2-amino-3-((tetrahydro-2H-pyran-4-yl)amino)benzoate I127

To a solution of methyl 2-nitro-3-((tetrahydro-2H-pyran-4-yl)amino)benzoate I126 (130 mg, 0.46 mmol) in EtOH (4 mL) was added 10% Pd/C (20 mg). The mixture was stirred under a hydrogen atmosphere at room temperature overnight. The catalyst was removed by filtration through Celite and the filtrate concentrated to give the title compound as a solid (100 mg, 86%). LCMS-C: Retention Time 2.32 min; m/z 251.1 [M+H]⁺.

### (d) Methyl 1-(tetrahydro-2H-pyran-4-yl)-JH-benzo[d]imidazole-4-carboxylate I128

To a solution of methyl 2-amino-3-((tetrahydro-2H-pyran-4-yl)amino)benzoate I127 (100 mg, 0.4 mmol) in trimethyl orthoformate (1 mL) was added TsOH H₂O (8 mg, 0.04 mmol). The reaction was heated at 100° C. for 2.5 hours then cooled to room temperature and poured into water (10 mL). The aqueous layer was extracted with EtOAc (10 mL×2), the combined organic fractions were dried (Na₂SO₄) and concentrated. The residue was purified by preparative TLC (6% MeOH/DCM) to give the title compound as a grey solid (45 mg, 43%). LCMS-C: Retention Time 0.33 min; m/z 261.1 [M+H]⁺.

### (e) 1-(Tetrahydro-2H-pyran-4-yl)-1H-benzo[d]imidazole-4-carboxylic acid I129

To a solution of methyl 1-(tetrahydro-2H-pyran-4-yl)-1H-benzo[d]imidazole-4-carboxylate I128 (300 mg, 1.1 mmol) in MeOH (5 mL) was added a solution of NaOH (90 mg, 2.2 mmol) in water (1 mL). The resulting mixture was stirred at room temperature overnight. The solvent was removed, and the residue re-suspended in water (5 mL). The pH of the aqueous solution was adjusted to 4-5 by addition of 1 M aqueous HCl solution. The precipitate was collected by filtration, washed with water (5 mL) and dried in air to give the title compound as a grey solid (150 mg, 53%). LCMS-C: Retention Time 0.66 min, m/z 247.1 [M+H]⁺

### (xlvi) 2-Methyl-1-(tetrahydro-2H-pyran-4-yl)-1H-benzo[d]imidazole-4-carboxylic acid I131

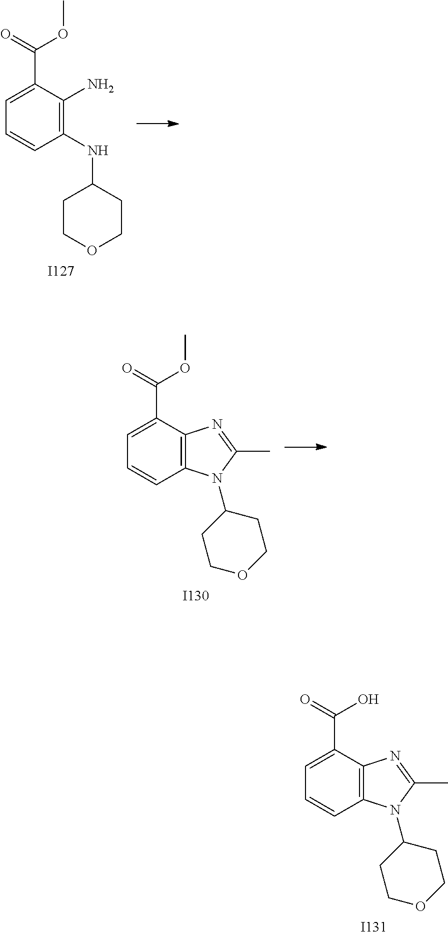 [SMILES: "I127" ; COC(=O)c1cccc(NC2CCOCC2)c1N ; "I130" ; COC(=O)c1cccc2c1nc(C)n2C1CCOCC1 ; "I131" ; Cc1nc2c(C(=O)O)cccc2n1C1CCOCC1]

### (a) Methyl 2-methyl-1-(tetrahydro-2H-pyran-4-yl)-1H-benzo[d]imidazole-4-carboxylate I130

To a solution of methyl 2-amino-3-((tetrahydro-2H-pyran-4-yl)amino)benzoate I127 (300 mg, 1.2 mmol) in triethyl orthoacetate (5 mL) was added TsOH H₂O (23 mg, 0.12 mmol). The mixture was heated at 100° C. for 3 hours then cooled to room temperature and poured into water (50 mL). The aqueous layer was extracted with EtOAc (50 mL×2) and the combined organic fractions washed with brine (100 mL), dried (Na₂SO₄) and concentrated. The residue was purified by column chromatography (2% MeOH/DCM) to give the title compound as a solid (200 mg, 52%). LCMS-C: Retention Time 0.34 min, m/z 275.1 [M+H]⁺. ¹H NMR (400 MHz, Methanol-d₄) δ 7.96 (d, J=8.4 Hz, 1H), 7.86 (d, J=8.4 Hz, 1H), 7.33 (t, J=8.4 Hz, 1H), 4.83-4.66 (m, 1H), 4.16-4.12 (m, 2H), 3.97 (s, 3H), 3.69-3.63 (m, 2H), 2.72 (s, 3H), 2.62-2.51 (m, 2H), 1.90-1.86 (m, 2H).

### (b) 2-Methyl-1-(tetrahydro-2H-pyran-4-yl)-1H-benzo[d]imidazole-4-carboxylic acid I131

To a solution of methyl 2-methyl-1-(tetrahydro-2H-pyran-4-yl)-1H-benzo[d]imidazole-4-carboxylate I130 (200 mg, 0.73 mmol) in MeOH (5 mL) was added a solution of NaOH (60 mg, 1.46 mmol) in water (1 mL). The resulting mixture was stirred at room temperature overnight. The solvent was removed, and the residue suspended in water (2 mL). The pH of the aqueous solution was adjusted to 4-5 by addition of 1 M aqueous HCl solution. The aqueous layer was washed with DCM (3 mL×2) and lyophilized to give the title compound as a solid (220 mg). The crude product was used in the next step without further purification. LCMS-C: Retention Time 0.31 min, m/z 261.1 [M+H]⁺

### (xlvii) Lithium 5-((1-(methoxycarbonyl)piperidin-4-yl)oxy)nicotinate I135

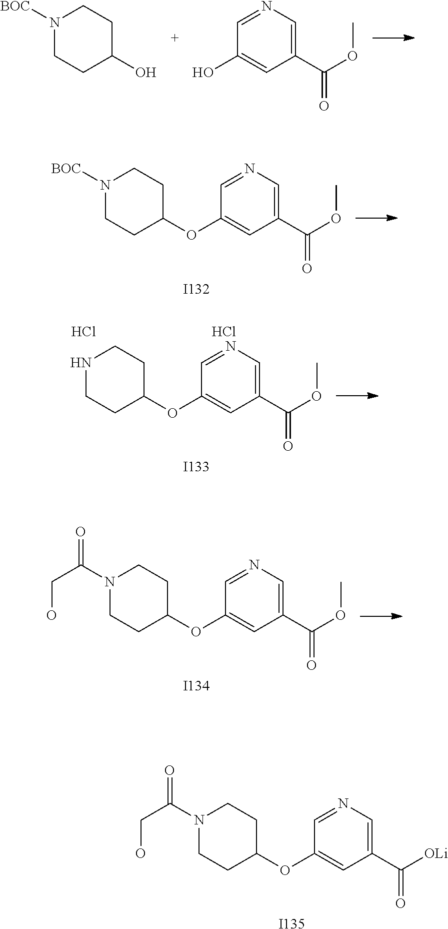

### (a) Methyl 5-((1-(tert-butoxycarbonyl)piperidin-4-yl)oxy)nicotinate I132

To a solution of methyl 5-hydroxynicotinate (500 mg, 3.3 mmol) in dry THF (10 mL) at 0° C. were added tert-butyl 4-hydroxypiperidine-1-carboxylate (660 mg, 3.3 mmol), DIAD (870 mg, 4.3 mmol), and PPh₃ (950 mg, 3.6 mmol). The mixture was stirred at 0° C. for 1 hour then allowed to warm slowly to room temperature and stirred overnight. The mixture was diluted with EtOAc (100 mL) and washed with water (100 mL) and brine (100 mL), dried (Na₂SO₄) and concentrated. The residue obtained was purified by column chromatography (20% EtOAc/petroleum ether) to give the title compound as oil (580 mg, 58%). LCMS-C: Retention Time 2.77 min, m/z 337.2 [M+H]⁺

### (b) Methyl 5-(piperidin-4-yloxy)nicotinate dihydrochloride I133

A mixture of methyl 5-((1-(tert-butoxycarbonyl)piperidin-4-yl)oxy)nicotinate I132 (570 mg, 1.7 mmol) in HCl in diethyl ether (2.0 M, 5 mL) was stirred at room temperature for 3 hours. The solvent was removed to give the title compound as a solid (500 mg, 96%). LCMS-C: Retention Time 0.31 min, m/z 237.1 [M+H]⁺

### (c) Methyl 5-((1-(methoxycarbonyl)piperidin-4-yl)oxy)nicotinate I134

To a solution of methyl 5-(piperidin-4-yloxy)nicotinate dihydrochloride I133 (500 mg, 1.6 mmol) in DCM (10 mL) was added Et₃N (520 mg, 5.1 mmol). Methyl chloroformate (185 mg, 1.9 mmol) was then added dropwise and the mixture stirred at room temperature for 1 hour. The mixture was diluted with DCM (50 mL) and washed with water (50 mL) and brine (50 mL), dried (Na₂SO₄) and concentrated. The residue was purified by column chromatography (5% methanol/DCM) to give the title compound as an oil (390 mg, 82%). ¹H NMR (400 MHz, Methanol-d₄) δ 8.71 (d, J=1.6 Hz, 1H), 8.47 (d, J=2.8 Hz, 1H), 7.92 (dd, J=2.8, 1.6 Hz, 1H), 4.80-4.74 (m, 1H), 3.95 (s, 3H), 3.80-3.74 (m, 2H), 3.70 (s, 3H), 3.46-3.40 (m, 2H), 2.04-1.98 (m, 2H), 1.78-1.70 (m, 2H). LCMS-C: Retention Time 2.34 min, m/z 295.1 [M+H]⁺

### d) Lithium 5-((1-(methoxycarbonyl)piperidin-4-yl)oxy)nicotinate I135

To a solution of methyl 5-((1-(methoxycarbonyl)piperidin-4-yl)oxy)nicotinate I134 (350 mg, 1.2 mmol) in a mixture of THF (3 mL), MeOH (6 mL) and water (1.5 mL) was added LiOH H₂O (50 mg, 2.4 mmol). The reaction was stirred at room temperature overnight then concentrated under reduced pressure. The residue obtained was re-dissolved in water (3 mL) and lyophilized to give the title compound as a solid (420 mg). LCMS-C: Retention Time 1.31 min; m/z 281.1 [M-Li+2H]⁺.

### (xlviii) 5-Methoxynicotinic acid I137

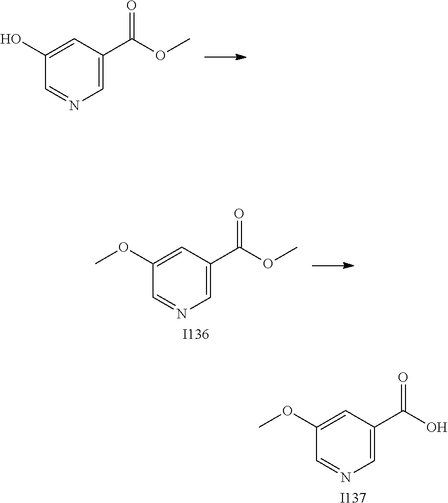

### (a) Methyl 5-methoxynicotinate I136

To a suspension of NaH (60% in mineral oil, 1.46 g, 0.03 mol, washed three times with hexane) in DMF (50 mL) was added methyl-5-hydroxynicotinate (4.0 g, 0.04 mol) portion-wise, keeping the temperature below 10° C. After 30 minutes, methyl iodide (3.87 g, 0.03 mol) was added dropwise over 20 minutes. The mixture was stirred at room temperature for 3 hours, then quenched with MeOH and concentrated in vacuo. The residue was dissolved in chloroform and partitioned against saturated NaHCO₃ and brine. The organic layer was separated and the aqueous layer was extracted with chloroform, the combined organic fractions were dried (Na₂SO₄) and concentrated in vacuo. The residue obtained was purified by flash silica gel chromatography (petroleum ether/EtOAc=5/1 then 3/1) to give the title compound (1.0 g, 23%) as a solid. LCMS-C: Retention Time 1.16 min; m/z 168.1 [M+H]⁺.

### (b) 5-Methoxynicotinic acid I137

To a solution of methyl 5-methoxynicotinate I136 (300 mg, 1.79 mmol) in a mixture of THF (4 mL) and methanol (4 mL) was added NaOH (2 M aqueous solution, 3 mL). The resulting mixture was stirred at room temperature for 14 hours. The solvent was removed, and the residue obtained diluted with water (20 mL). The pH of the aqueous mixture was adjusted to 6 by addition of a 2 M aqueous HCl solution. The mixture was extracted with DCM (20 mL×3) and the combined organic layers washed with brine (10 mL×2), dried (Na₂SO₄) and concentrated to give the title compound as a solid (230 mg, 84%). LCMS-C: Retention Time 0.36 min; m/z 154.1 [M+H]⁺.

### (xlix) Alternate synthesis of (S)-tert-Butyl 3-((R)-2-(6-chloronicotinamido)-1-hydroxyethyl)-3,4-dihydroisoquinoline-2(1H)-carboxylate A19

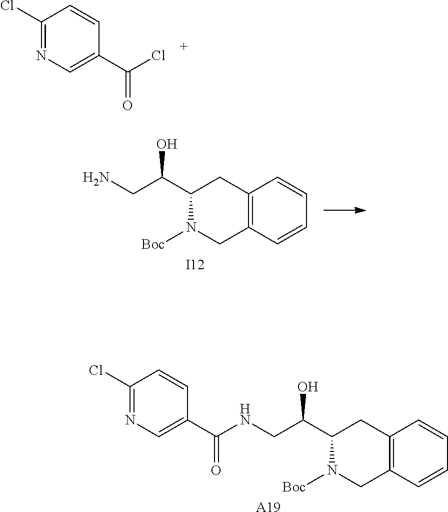

To a solution of (S)-tert-butyl 3-((R)-2-amino-1-hydroxyethyl)-3,4-dihydroisoquinoline-2(1H)-carboxylate I12 (500 mg, 1.71 mmol, 1.0 eq) in DCM (10 mL) at 0° C. was added Et₃N (520 mg, 5.13 mmol, 3.0 eq) and 6-chloronicotinoyl chloride (301 mg, 1.71 mmol, 1.0 eq). The reaction was stirred at room temperature under a nitrogen atmosphere overnight. Saturated aqueous NaHCO₃ (50 mL) was added and the resulting mixture extracted with DCM (3×50 mL). The combined organic fractions were dried (Na₂SO₄) and concentrated, and the crude residue purified by chromatography (25% EtOAc/petroleum ether) to give the title compound (510 mg, 69%) as a solid. LCMS-C: Retention Time 2.87 min; m/z 454.2 [M+Na]⁺.

### (1) 5-Isopropoxynicotinic acid I39

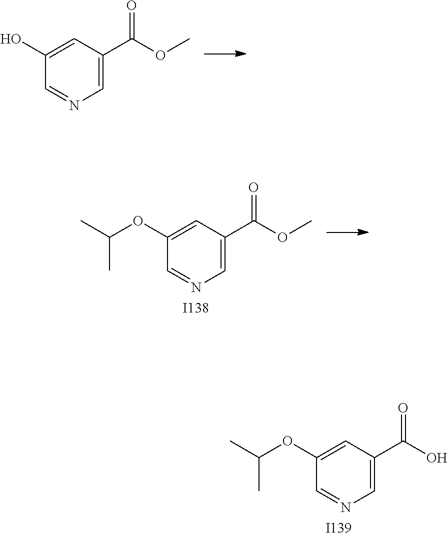

### (a) Methyl 5-isopropoxynicotinate I38

A mixture of DCC (1.62 g, 7.84 mmol) and CuCl (129 mg, 1.31 mmol) in isopropanol (20 mL) was heated at 60° C. After 13 hours, 5-hydroxynicotinic acid methyl ester (1.0 g, 6.53 mmol) and benzene (20 mL) were added and the reaction heated at 105° C. for 24 hours. The mixture was diluted with chloroform and filtered. The organic filtrate was washed with saturated NaHCO₃ (15 mL), water and brine, dried (Na₂SO₄) and concentrated. Purification by flash silica gel chromatography (EtOAc/petroleum ether=1/6) gave the title compound (300 mg, 24%) as a solid. LCMS-C: Retention Time 2.32 min; m/z 196.1 [M+H]⁺.

### (b) 5-Isopropoxynicotinic acid I39

To a solution of methyl 5-isopropoxynicotinate I38 (300 mg, 1.54 mmol) in a mixture of THF (2 mL) and methanol (4 mL) was added NaOH (2M aqueous solution, 2 mL). The resulting mixture was stirred at room temperature for 14 hours. The solvent was removed, and the residue diluted with water (20 mL). The pH of the aqueous mixture was adjusted to 6 by addition of a 2M aqueous HCl solution. The mixture was extracted with DCM (20 mL×3) and the combined organic layers washed with brine (10 mL×2), dried (Na₂SO₄) and concentrated to give the title compound (250 mg, 90%) as a solid. LCMS-C: Retention Time 0.88 min; m/z 182.1 [M+H]⁺.

### PRMT5 Compound Examples

The following experimental procedures detail the preparation of specific PRMT5 inhibitor compounds useful in the present invention. The examples are for illustrative purposes only and are not intended to limit the scope of the instant disclosure in any way. These disclosed PRMT5 compounds, as representative compounds, are useful in the methods in treating a patient who is predicted to be responsive to treatment, wherein the patient has the presence of at least one of the following:
a) a FLT3 internal tandem duplication; or
b) a mutation in NPM1 or DNMT3a; or
c) a mutation in any of the following splicing genes SRSF2, ZRSR2, or SF3B1.

These disclosed PRMT5 compounds, as representative compounds, are useful in a method of identifying a patient diagnosed with cancer predicted to be responsive to a treatment with PRMT5 inhibitor comprising;
a) obtaining a biological sample comprising cancer cells from a patient diagnosed with cancer;
b) measuring the gene expression level of a predictive biomarker of PRMT5 inhibitor responsiveness in the biological sample;
c) identifying a patient predicted to be responsive to a treatment with a PRMT5 inhibitor, wherein the patients has the presence of a least one of the following:
   i. a FLT3 internal tandem duplication; or
   ii. a mutation in NPM1 or DNMT3a; or
   iii. a mutation in any of the following splicing genes SRSF2, ZRSR2, or SF3B1; and
d) administering an effective amount of a PRMT5 inhibitor, or a pharmaceutically acceptable salt thereof, to the patient, if the predictive biomarker is present.

The substituted reagents and starting material were commercially acquired, synthesized as reported above, or synthesized through known routes reported in the literature.

### Example 1

### (1R,2S,3R,5R)-5-(((2-amino-3-bromoquinolin-7-yl)oxy)methyl)-3-(4-chloro-7H-pyrrolo[2,3-d]pyrimidin-7-yl)-1-methylcyclopentane-1,2-diol

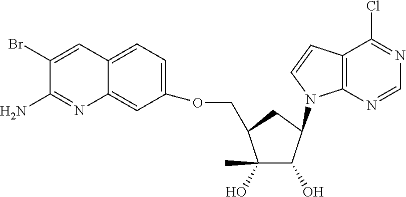 [SMILES: C[C@@]1(O)[C@@H](COc2ccc3cc(Br)c(N)nc3c2)C[C@@H](n2ccc3c(Cl)ncnc32)[C@@H]1O]

Step 1: A mixture of ((3aR,4R,6R,6aS)-6-(4-chloro-7H-pyrrolo[2,3-d]pyrimidin-7-yl)-2,2,3a-trimethyltetrahydro-3aH-cyclopenta[d][1,3]dioxol-4-yl)methanol (1.2 g, 3.6 mmol), 2-amino-3-bromoquinolin-7-ol (0.934 g, 3.91 mmol), and triphenylphosphine (1.86 g, 7.10 mmol) was co-evaporated with dry toluene (three times, 10 mL each) and then re-dissolved in anhydrous THF (20 mL). The reaction mixture was cooled to 0° C., and (E)-diisopropyl diazene-1,2-dicarboxylate (1.44 g, 7.10 mmol) was added dropwise at 0° C. The mixture was warmed to room temperature naturally, and the mixture was stirred at room temperature for 2 hours. The reaction mixture was concentrated under reduced pressure. The residue was purified by silica gel column chromatography (0-10% MeOH in DCM). The fractions containing the desired product were combined and concentrated under reduced pressure to afford a crude solid. The crude material was further purified by reverse-phase column chromatography (0-100% 5 mM aqueous NH₄HCO₃/acetonitrile) to afford 3-bromo-7-(((3aR,4R,6R,6aS)-6-(4-chloro-7H-pyrrolo[2,3-d]pyrimidin-7-yl)-2,2,3a-trimethyltetrahydro-3aH-cyclopenta[d][1,3]dioxol-4-yl)methoxy)quinolin-2-amine. MS 558, 560 (M+1, M+3). ¹H NMR (400 MHz, DMSO-d6) δ 8.68 (d, J=1.2 Hz, 1H), 8.28 (s, 1H), 7.96 (d, J=3.6 Hz, 1H), 7.58 (d, J=8.8 Hz, 1H), 6.98 (d, J=2.4 Hz, 1H), 6.90 (dd, J=9.2, 2.4 Hz, 1H), 6.76 (d, J=3.6 Hz, 1H), 6.53 (s, 2H), 5.40-5.13 (m, 1H), 4.51 (d, J=4.0 Hz, 1H), 4.27 (dd, J=10.0, 6.0 Hz, 1H), 4.12 (t, J=8.8 Hz, 1H), 2.72-2.66 (m, 1H), 2.49-2.45 (m, 1H), 2.42-2.36 (m, 1H), 1.60 (s, 3H), 1.51 (s, 3H), 1.30 (s, 3H).

Step 2: To a solution of 3-bromo-7-(((3aR,4R,6R,6aS)-6-(4-chloro-7H-pyrrolo[2,3-d]pyrimidin-7-yl)-2,2,3a-trimethyltetrahydro-3aH-cyclopenta[d][1,3]dioxol-4-yl)methoxy)quinolin-2-amine (430 mg, 0.769 mmol) in water (8 mL) was added TFA (8 mL) at room temperature. The reaction was stirred at 25° C. for 4 h. The reaction was cooled to 0° C. The pH was adjusted to pH 7-8 with saturated aqueous sodium bicarbonate (50 mL). The resultant mixture was extracted with EtOAc (50 mL×5) and the combined organic layers were dried over anhydrous sodium sulfate, filtered, and concentrated under reduced pressure. The residue was purified by silica gel column chromatography (0-15% of MeOH in DCM) to afford (1R,2S,3R,5R)-5-(((2-amino-3-bromoquinolin-7-yl)oxy)methyl)-3-(4-chloro-7H-pyrrolo[2,3-d]pyrimidin-7-yl)-1-methylcyclopentane-1,2-diol. MS 518, 520 (M+1, M+3). ¹H NMR (400 MHz, DMSO-d6) δ 8.60 (s, 1H), 8.28 (s, 1H), 7.93 (d, J=4.0 Hz, 1H), 7.58 (d, J=8.8 Hz, 1H), 6.97 (s, 1H), 6.93 (d, J=8.8 Hz, 1H), 6.71 (d, J=3.6 Hz, 1H), 6.51 (br s, 2H), 5.13 (q, J=9.2 Hz, 1H), 4.99 (d, J=7.2 Hz, 1H), 4.51 (s, 1H), 4.21-4.11 (m, 3H), 2.47-2.39 (m, 2H), 1.82-1.75 (m, 1H), 1.26 (s, 3H).

### Example 2

### (1R,2S,3R,5R)-5-(((2-amino-3-bromoquinolin-7-yl)oxy)methyl)-3-(4-amino-7H-pyrrolo[2,3-d]pyrimidin-7-yl)-1-methylcyclopentane-1,2-diol

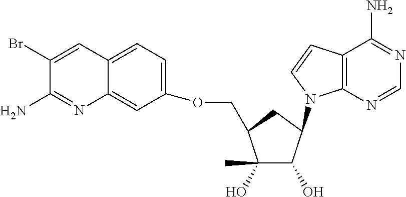 [SMILES: C[C@@]1(O)[C@@H](COc2ccc3cc(Br)c(N)nc3c2)C[C@@H](n2ccc3c(N)ncnc32)[C@@H]1O]

To a sealed tube (10 mL) was added (1R,2S,3R,5R)-5-(((2-amino-3-bromoquinolin-7-yl)oxy)methyl)-3-(4-chloro-7H-pyrrolo[2,3-d]pyrimidin-7-yl)-1-methylcyclopentane-1,2-diol (350 mg, 0.675 mmol), 1,4-dioxane (3 mL) and NH₃H₂O (5 mL; 25%-28% w/w) at room temperature. The reaction mixture was sealed tightly and then stirred at 90° C. for 16 hours. The reaction mixture was concentrated under reduced pressure. The residue was purified by reverse phase HPLC (0-45% acetonitrile/water) to afford (1R,2S,3R,5R)-5-(((2-amino-3-bromoquinolin-7-yl)oxy)methyl)-3-(4-amino-7H-pyrrolo[2,3-d]pyrimidin-7-yl)-1-methylcyclopentane-1,2-diol. MS 499, 501 (M+1, M+3). ¹H NMR (400 MHz, DMSO-d₆) δ 8.28 (s, 1H), 8.02 (s, 1H), 7.58 (d, J=8.7 Hz, 1H), 7.30 (d, J=3.6 Hz, 1H), 6.96-6.89 (m, 4H), 6.57-6.53 (m, 3H), 4.97-4.91 (m, 2H), 4.41 (s, 1H), 4.19-4.10 (m, 3H), 2.45-2.37 (m, 2H), 1.72-1.66 (m, 1H), 1.24 (s, 3H).

### Example 3

### (2R,3S,4R,5R)-2-(((2-amino-3-bromoquinolin-7-yl)oxy)methyl)-5-(4-amino-7H-pyrrolo[2,3-d]pyrimidin-7-yl)-3-ethynyltetrahydrofuran-3,4-diol

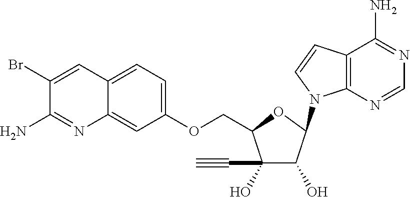 [SMILES: C#C[C@@]1(O)[C@@H](COc2ccc3cc(Br)c(N)nc3c2)O[C@@H](n2ccc3c(N)ncnc32)[C@@H]1O]

Step 1: (2R,3R,4S,5R)-2-(4-amino-7H-pyrrolo[2,3-d]pyrimidin-7-yl)-5-(hydroxymethyl)tetrahydrofuran-3,4-diol (2 g, 7.5 mmol) was co-evaporated with dry pyridine (10 mL×3) and then re-suspended in dry pyridine (30 mL) at ambient temperature under an argon atmosphere. To this suspension was added chlorotrimethylsilane (5.71 g, 52.6 mmol) in one portion at 0° C., and the mixture was maintained at ambient temperature for 1 hour. Then to the mixture was added benzoyl chloride (1.58 g, 11.3 mmol) at 0° C. After stirring at ambient temperature for 2 h, the resulting mixture was quenched with H₂O (8 mL) at 0° C. Then aqueous NH₃ solution (15 mL, 25-28% wt) was added dropwise at 0° C. followed by stirring at ambient temperature for 30 min. The reaction mixture was concentrated under reduced pressure, and the residue was purified by column chromatography on silica (6% MeOH in DCM) to afford N-(7-((2R,3R,4S,5R)-3,4-dihydroxy-5-(hydroxymethyl)tetrahydrofuran-2-yl)-7H-pyrrolo[2,3-d]pyrimidin-4-yl)benzamide. MS: 371 (M+1). ¹H NMR (300 MHz, DMSO-d₆) δ 11.12 (s, 1H), 8.59 (s, 1H), 8.10-8.00 (m, 2H), 7.71 (d, J=3.8 Hz, 1H), 7.63 (t, J=7.3 Hz, 1H), 7.53 (dd, J=8.3, 6.6 Hz, 2H), 6.67 (d, J=3.8 Hz, 1H), 6.22 (d, J=6.1 Hz, 1H), 5.35 (d, J=6.4 Hz, 1H), 5.15 (d, J=4.8 Hz, 1H), 5.06 (t, J=5.4 Hz, 1H), 4.41 (dd, J=5.9 Hz, 1H), 4.10 (dd, J=4.5 Hz, 1H), 3.91 (d, J=3.7 Hz, 1H), 3.69-3.47 (m, 2H).

Step 2: N-(7-((2R,3R,4S,5R)-3,4-dihydroxy-5-(hydroxymethyl)tetrahydrofuran-2-yl)-7H-pyrrolo[2,3-d]pyrimidin-4-yl)benzamide (2.1 g, 5.7 mmol) was co-evaporated with dry pyridine (10 mL×3) and then re-suspended in dry pyridine (15 mL) at ambient temperature under an argon atmosphere. To this suspension was added 4,4′-(chloro(phenyl)methylene)bis(methoxy-benzene) (2.11 g, 6.24 mmol) in one portion at ambient temperature, and the mixture was maintained at ambient temperature for 2 hours. The reaction mixture was concentrated under reduced pressure, and the residue was purified by column chromatography on silica (EtOAc in petroleum ether) to give N-(7-((2R,3R,4S,5R)-5-((bis(4-methoxyphenyl)(phenyl)methoxy)-methyl)-3,4-dihydroxytetrahydrofuran-2-yl)-7H-pyrrolo[2,3-d]pyrimidin-4-yl)benzamide as a solid. MS: 673 (M+1). ¹H-NMR (300 MHz, DMSO-d₆) δ 11.13 (s, 1H), 8.59 (s, 1H), 8.15-7.94 (m, 2H), 7.68-7.48 (m, 4H), 7.37 (d, J=7.6 Hz, 2H), 7.31-7.15 (m, 7H), 6.84 (dd, J=8.7, 1.6 Hz, 4H), 6.65 (d, J=3.7 Hz, 1H), 6.25 (d, J=5.0 Hz, 1H), 5.47 (d, J=5.9 Hz, 1H), 5.19 (d, J=5.6 Hz, 1H), 4.46 (dd, J=5.5 Hz, 1H), 4.20 (dd, J=5.2 Hz, 1H), 3.99 (d, J=7.1 Hz, 1H), 3.71 (s, 6H), 3.23-3.19 (m, 2H).

Step 3: N-(7-((2R,3R,4S,5R)-5-((bis(4-methoxyphenyl)(phenyl)methoxy)methyl)-3,4-dihydroxytetrahydrofuran-2-yl)-7H-pyrrolo[2,3-d]pyrimidin-4-yl)benzamide (2.36 g, 3.51 mmol), (2R,4S)-4-isopropyl-2-methoxy-3-((R)-2-methyl-1-(1-methyl-1H-imidazol-2-yl)propyl)oxazolidine (0.20 g, 0.70 mmol), and N-ethyl-N-isopropylpropan-2-amine hydrochloride (0.017 g, 0.105 mmol) were co-evaporated with dry toluene (10 mL×3) and then re-suspended in dry THF (21 mL) under an argon atmosphere. To the suspension was added N-ethyl-N-isopropylpropan-2-amine (2.13 g, 16.5 mmol) in one portion at 0° C. This was followed by addition of triisopropylsilyl trifluoromethanesulfonate (4.62 g, 15.1 mmol) in DCM (10 mL) at 0° C. The reaction was stirred at ambient temperature for 40 min. The reaction mixture was then quenched with water (10 mL) and extracted with EtOAc (2×100 mL). The combined organic layers were washed with saturated aqueous NaHCO₃ (50 mL) and brine (2×50 mL), dried over anhydrous sodium sulfate, and filtered. The filtrate was concentrated under reduced pressure, and the residue was purified by column chromatography on silica (25% EtOAc in petroleum ether) to afford N-(7-((2R,3R,4R,5R)-5-((bis(4-methoxyphenyl)(phenyl)methoxy)-methyl)-4-hydroxy-3-((triisopropylsilyl)oxy)tetrahydrofuran-2-yl)-7H-pyrrolo[2,3-d]pyrimidin-4-yl)benzamide. MS: 829 (M+1). ¹H NMR (300 MHz, DMSO-d₆) δ 11.10 (s, 1H), 8.55 (s, 1H), 8.19-7.95 (m, 2H), 7.68-7.48 (m, 4H), 7.45-7.35 (m, 2H), 7.25 (dd, J=7.2, 5.4 Hz, 7H), 6.89-6.80 (m, 4H), 6.68 (d, J=3.7 Hz, 1H), 6.32 (d, J=5.6 Hz, 1H), 5.11 (d, J=6.1 Hz, 1H), 4.77 (t, J=5.4 Hz, 1H), 4.26-4.06 (m, 2H), 3.71 (s, 6H), 3.28-3.17 (m, 2H), 0.98-0.87 (m, 12H), 0.82 (d, J=6.4 Hz, 9H).

Step 4: N-(7-((2R,3R,4R,5R)-5-((bis(4-methoxyphenyl)(phenyl)methoxy)methyl)-4-hydroxy-3-((triisopropylsilyl)oxy)tetrahydrofuran-2-yl)-7H-pyrrolo[2,3-d]pyrimidin-4-yl)benzamide (1.95 g, 2.35 mmol) was co-evaporated with dry pyridine (10 mL×3) and then re-suspended in dry DCM (30 mL) at ambient temperature under an argon atmosphere. To this suspension was added Dess-Martin periodinane (2.49 g, 5.88 mmol) and pyridine (0.65 g, 8.2 mmol) at ambient temperature. The resulting mixture was then stirred at 0° C. for 2 hours. The reaction mixture was quenched with saturated aqueous NaHCO₃ (10 mL) and then extracted with EtOAc (100 mL×2). The combined organic layers were washed with brine (100 mL), dried over anhydrous sodium sulfate, and filtered. The filtrate was concentrated under reduced pressure to give crude N-(7-((2R,3S,5R)-5-((bis(4-methoxyphenyl)(phenyl)methoxy)methyl)-4-oxo-3-((triisopropylsilyl)-oxy)tetrahydrofuran-2-yl)-7H-pyrrolo[2,3-d]pyrimidin-4-yl)benzamide, which was used directly in the next step. MS: 827 (M+1).

Step 5: To a solution of N-(7-((2R,3S,5R)-5-((bis(4-methoxyphenyl)(phenyl)methoxy)methyl)-4-oxo-3-((triisopropylsilyl)oxy)tetrahydrofuran-2-yl)-7H-pyrrolo[2,3-d]pyrimidin-4-yl)benzamide (2.27 g, 2.74 mmol) in DCM (40 mL) was added 2,2-dichloroacetic acid (3.18 g, 24.7 mmol) at ambient temperature and then stirred for 30 minutes. Triethylsilane (31.9 g, 274 mmol) was added to this suspension. After stirring for an additional 10 minutes, pyridine (1.5 mL) was added to the mixture. Then the mixture was concentrated under reduced pressure, and the residue was purified by column chromatography on silica (30% EtOAc in petroleum ether) to afford N-(7-((2R,3S,5R)-5-(hydroxymethyl)-4-oxo-3-((triisopropylsilyl)oxy)tetrahydrofuran-2-yl)-7H-pyrrolo[2,3-d]pyrimidin-4-yl)benzamide. MS: 525 (M+1). ¹H NMR (400 MHz, DMSO-d₆) δ 11.20 (s, 1H), 8.65 (s, 1H), 8.14-8.04 (m, 2H), 7.89 (d, J=3.8 Hz, 1H), 7.71-7.62 (m, 1H), 7.61-7.53 (m, 2H), 6.84 (d, J=3.8 Hz, 1H), 6.53 (d, J=8.3 Hz, 1H), 5.39 (br, 1H), 5.00 (d, J=8.3 Hz, 1H), 4.44 (t, J=2.8 Hz, 1H), 3.74-3.70 (m, 2H), 0.91-0.87 (m, 12H), 0.78-0.73 (m, 9H).

Step 6: Cerium (III) chloride (3.04 g, 12.4 mmol) was dried at 140° C. under reduced pressure for 1 h. The resulting powder was cooled under argon. Anhydrous THF (20 mL) was added. The resulting mixture was cooled to −78° C., and ((trimethylsilyl)ethynyl)lithium (24.7 mL, 12.4 mmol) was added. The reaction mixture was stirred for 1 h at −78° C. Then a cooled solution (−78° C.) of N-(7-((2R,3S,5R)-5-(hydroxymethyl)-4-oxo-3-((triisopropylsilyl)oxy)tetrahydrofuran-2-yl)-7H-pyrrolo[2,3-d]pyrimidin-4-yl)benzamide (1.08 g, 2.06 mmol) in anhydrous THF (20 mL) was rapidly added, and the stirring was continued for 2 h. The reaction was quenched with saturated aqueous ammonium chloride solution (40 mL). The mixture was diluted with EtOAc (300 mL) and washed with water (100 mL×2). The organic fraction was dried over anhydrous sodium sulfate and filtered. The filtrate was concentrated under reduced pressure, and the residue was purified by reverse phase HPLC (0-95% 5 mM aqueous NH₄HCO₃/acetonitrile) to afford N-(7-((2R,3R,4R,5R)-4-hydroxy-5-(hydroxymethyl)-3-((triisopropylsilyl)oxy)-4-((trimethylsilyl)ethynyl)tetrahydrofuran-2-yl)-7H-pyrrolo[2,3-d]pyrimidin-4-yl)benzamide. MS: 623 (M+1). ¹H NMR (300 MHz, DMSO-d₆) δ 11.08 (s, 1H), 8.56 (s, 1H), 8.06-8.03 (m, 2H), 7.80 (d, J=3.8 Hz, 1H), 7.69-7.41 (m, 3H), 6.71 (d, J=3.7 Hz, 1H), 6.29 (d, J=7.1 Hz, 1H), 5.80 (s, 1H), 5.18 (t, J=4.6 Hz, 1H), 4.98 (d, J=7.2 Hz, 1H), 3.98 (t, J=3.2 Hz, 1H), 3.87-3.60 (m, 2H), 0.85-0.70 (m, 21H), 0.14 (s, 9H).

Step 7: To a mixture of N-(7-((2R,3R,4R,5R)-4-hydroxy-5-(hydroxymethyl)-3-((triisopropylsilyl)oxy)-4-((trimethylsilyl)ethynyl)tetrahydrofuran-2-yl)-7H-pyrrolo[2,3-d]pyrimidin-4-yl)benzamide (250 mg, 0.401 mmol) in THF (8 mL) was added 2-amino-3-bromoquinolin-7-ol (115 mg, 0.482 mmol) and triphenylphosphine (368 mg, 1.41 mmol) under an argon atmosphere. Then (E)-diisopropyl diazene-1,2-dicarboxylate (203 mg, 1.00 mmol) was added dropwise at 0° C. The mixture was stirred at ambient temperature for 12 h. The reaction mixture was filtered, and the filtrate was concentrated under reduced pressure. The residue was purified by column chromatography on silica (50% EtOAc in petroleum ether) to afford N-(7-((2R,3R,4R,5R)-5-(((2-amino-3-bromoquinolin-7-yl)oxy)methyl)-4-hydroxy-3-((triisopropylsilyl)oxy)-4-((trimethylsilyl)ethynyl)tetrahydrofuran-2-yl)-7H-pyrrolo[2,3-d]pyrimidin-4-yl)benzamide. MS: 843/845 (M+1/M+3).

Step 8: To a mixture of N-(7-((2R,3R,4R,5R)-5-(((2-amino-3-bromoquinolin-7-yl)oxy)methyl)-4-hydroxy-3-((triisopropylsilyl)oxy)-4-((trimethylsilyl)ethynyl)tetrahydrofuran-2-yl)-7H-pyrrolo[2,3-d]pyrimidin-4-yl)benzamide (90 mg, 0.11 mmol) in pyridine (3 mL) was added triethylamine (1.08 g, 10.7 mmol) and triethylamine trihydrofluoride (860 mg, 5.33 mmol) at ambient temperature. Stirring was then continued at ambient temperature for 1 h. The reaction mixture was concentrated under reduced pressure, and the residue was purified by column chromatography on silica (90% EtOAc in petroleum ether) to afford N-(7-((2R,3R,4S,5R)-5-(((2-amino-3-bromoquinolin-7-yl)oxy)methyl)-4-ethynyl-3,4-dihydroxytetrahydrofuran-2-yl)-7H-pyrrolo[2,3-d]pyrimidin-4-yl)benzamide. MS: 615/617 (M+1/M+3). ¹H NMR (300 MHz, CD₃OD) δ 8.57 (s, 1H), 8.21 (s, 1H), 8.06-8.00 (m, 2H), 7.73 (d, J=3.8 Hz, 1H), 7.68-7.60 (m, 1H), 7.58 (d, J=4.2 Hz, 1H), 7.56-7.53 (m, 1H), 7.52-7.32 (m, 1H), 7.10-7.00 (m, 2H), 6.88 (d, J=3.8 Hz, 1H), 6.50 (d, J=7.4 Hz, 1H), 4.94 (d, J=7.4 Hz, 1H), 4.54-4.41 (m, 3H), 3.15 (s, 1H).

Step 9: To a mixture of N-(7-((2R,3R,4S,5R)-5-(((2-amino-3-bromoquinolin-7-yl)oxy)methyl)-4-ethynyl-3,4-dihydroxytetrahydrofuran-2-yl)-7H-pyrrolo[2,3-d]pyrimidin-4-yl)benzamide (40 mg, 0.065 mmol) in MeOH (2 mL) was added sodium methanolate (17.6 mg, 0.325 mmol) at ambient temperature. Stirring was then continued at ambient temperature for 16 h. The reaction mixture was concentrated under reduced pressure, and the residue was purified by reverse phase column chromatography (ACN/water with 5 mM aqueous NH₄HCO₃ modifier) to afford (2R,3S,4R,5R)-2-(((2-amino-3-bromoquinolin-7-yl)oxy)methyl)-5-(4-amino-7H-pyrrolo[2,3-d]pyrimidin-7-yl)-3-ethynyltetrahydrofuran-3,4-diol. MS: 511/513 (M+1/M+3). ¹H NMR (300 MHz, DMSO-d₆) δ 8.30 (s, 1H), 8.10 (s, 1H), 7.60 (d, J=9.5 Hz, 1H), 7.45 (d, J=3.7 Hz, 1H), 7.22 (br s, 2H), 6.95-6.93 (m, 2H), 6.76-6.60 (m, 3H), 6.22 (s, 1H), 6.16 (d, J=7.5 Hz, 1H), 5.95 (d, J=7.3 Hz, 1H), 4.74 (t, J=7.3 Hz, 1H), 4.35-4.29 (m, 3H), 3.61 (s, 1H).

### Example 4

### (2R,3S,4R,5R)-2-(2-(2-amino-3-bromoquinolin-7-yl)ethyl)-5-(4-amino-7H-pyrrolo[2,3-d]pyrimidin-7-yl)-2,3-dimethyltetrahydrofuran-3,4-diol

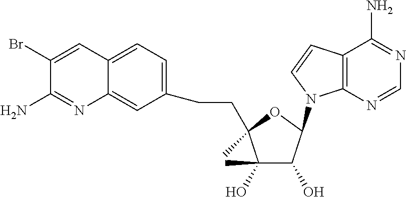 [SMILES: C[C@]1(O)[C@@H](O)[C@H](n2ccc3c(N)ncnc32)O[C@@]1(C)CCc1ccc2cc(Br)c(N)nc2c1]

Step 1: To a stirred solution of ((3aR,5R,6S,6aR)-5-((tert-butyldiphenylsilyloxy)methyl)-2,2-dimethyl-6-(naphthalen-2-ylmethoxy)-tetrahydrofuro[3,2-d][1,3]dioxol-5-yl)methanol (12 g, 20 mmol) and imidazole (5.44 g, 80 mmol) in toluene (240 mL) was added PPh₃ (21 g, 80 mmol) at 25° C. under an argon atmosphere. Then 1₂ (10.1 g, 40 mmol) was added in portions to the mixture at 60° C. The resulting mixture was stirred at 80° C. for 14 h. The reaction mixture was cooled to 0° C., quenched with saturated aqueous Na₂S203 (200 mL) and diluted with EtOAc (300 mL). The organic layer was washed with H₂O (150 mL), saturated aqueous NaHCO₃ (150 mL×2) and brine (150 mL). The organic layer was dried over anhydrous Na₂SO₄, filtered, and the filtrate was concentrated under reduced pressure. The residue was purified by silica gel column chromatography (10-40% EtOAc/pet. ether) to afford tert-butyl(((3aR,5R,6S,6aR)-5-(iodomethyl)-2,2-dimethyl-6-(naphthalen-2-ylmethoxy)-tetrahydrofuro[3,2-d][1,3]dioxol-5-yl)methoxy)diphenylsilane. MS: 726 (M+NH₄). ¹H NMR (300 MHz, DMSO-d₆): δ 7.92-7.68 (m, 4H), 7.60-7.25 (m, 13H), 5.68 (d, J=3.6 Hz, 1H), 4.98-4.82 (m, 2H), 4.71 (d, J=12.3 Hz, 1H), 4.41 (d, J=2.4 Hz, 1H), 3.87 (d, J=12.0 Hz, 1H), 3.71-3.50 (m, 3H), 1.50 (s, 3H), 1.27 (s, 3H), 0.81 (s, 9H).

Step 2: Tert-butyl (((3aR,5R,6S,6aR)-5-(iodomethyl)-2,2-dimethyl-6-(naphthalen-2-ylmethoxy)-tetrahydrofuro[3,2-d][1,3]dioxol-5-yl)methoxy)diphenylsilane (10.6 g, 14.6 mmol) (co-evaporated with freshly distilled toluene (10 mL×3)) was dissolved in 200 mL of toluene. (Z)-3,3′-(diazene-1,2-diyl)bis(2,2-dimethyl-3-oxopropanenitrile) (615 mg, 3.85 mmol) and (n-Bu)₃SnH (11 g, 37 mmol) were added at 60° C. under an argon atmosphere in one portion; then the temperature was increased to 120° C., and the reaction was stirred at this temperature for 3 h. The reaction mixture was cooled to room temperature and diluted with EtOAc (300 mL). The organic layer was washed with H₂O (150 mL), saturated aqueous NaHCO₃ (150 mL×2) and brine (150 mL). The organic layer was dried over anhydrous Na₂SO₄, filtered, and the filtrate was concentrated under the reduced pressure. The residue was purified by silica gel column chromatography (14% EtOAc/pet. ether) to afford tert-butyldiphenyl(((3aR,5R,6S,6aR)-2,2,5-trimethyl-6-(naphthalen-2-ylmethoxy)-tetrahydrofuro[3,2-d][1,3]dioxol-5-yl)methoxy)silane. MS: 600 (M+NH₄). ¹H NMR (300 MHz, DMSO-d₆): δ 7.91-7.84 (m, 4H), 7.56-7.49 (m, 7H), 7.45-7.34 (m, 6H), 5.70 (d, J=3.9 Hz, 1H), 4.94-4.83 (m, 2H), 4.67 (d, J=12.3 Hz, 1H), 4.16 (d, J=5.1 Hz, 1H), 3.41 (dd, J=21.0, 9.0 Hz, 2H), 1.52 (s, 3H), 1.30 (s, 3H), 1.26 (s, 3H), 0.82 (s, 9H).

Step 3: To a solution of tert-butyldiphenyl(((3aR,5R,6S,6aR)-2,2,5-trimethyl-6-(naphthalen-2-ylmethoxy)tetrahydrofuro[2,3-d][1,3]dioxol-5-yl)methoxy)silane (5 g, 8.6 mmol) in DCM (50 mL) and water (12.5 mL) was added DDQ (3.90 g, 17.2 mmol) at ambient temperature. The resulting mixture was stirred at room temperature for 2 h. The reaction mixture was quenched with saturated aqueous NaHCO₃ (100 mL) and extracted with DCM (100 mL×3). The combined organic layers were washed with saturated aqueous NaHCO₃ (150 mL), dried over anhydrous Na₂SO₄, and filtered. The filtrate was concentrated under reduced pressure, and the residue was purified by silica gel chromatography (1-20% EtOAc/pet. ether) to afford (3aR,5R,6S,6aR)-5-(((tert-butyldiphenylsilyl)oxy)methyl)-2,2,5-trimethyltetrahydrofuro[2,3-d][1,3]dioxol-6-ol. MS: 460 (M+NH₄). ¹H NMR (300 MHz, DMSO-d₆) δ 7.61-7.42 (m, 10H), 5.65 (d, J=3.6 Hz, 1H), 5.11 (d, J=6.6 Hz, 1H), 4.57 (t, J=4.8 Hz, 1H), 4.16 (t, J=6.0 Hz, 1H), 3.49-3.40 (m, 2H), 1.48 (s, 3H), 1.25 (s, 3H), 1.14 (s, 3H), 0.99 (s, 9H).

Step 4: To a stirred solution of (3aR,5R,6S,6aR)-5-(((tert-butyldiphenylsilyl)oxy)methyl)-2,2,5-trimethyltetrahydrofuro[2,3-d][1,3]dioxol-6-ol (3.5 g, 7.91 mmol) in DCM (80 mL) was added pyridine (2.24 mL, 27.7 mmol) and Dess Martin Periodinane (6.71 g, 15.8 mmol) at 0° C. The resulting mixture was stirred at room temperature for 2 h. The reaction mixture was quenched with saturated aqueous NaHCO₃ (50 mL) and extracted with EtOAc (80 mL×3). The combined organic layers were washed with saturated aqueous NaHCO₃ (80 mL) and brine (100 mL), dried over anhydrous sodium sulfate, and filtered. The filtrate was concentrated under reduced pressure, and the residue was purified by silica gel column chromatography (1-24% EtOAc/pet. ether) to afford (3aR,5R,6aS)-5-(((tert-butyldiphenylsilyl)oxy)methyl)-2,2,5-trimethyldihydrofuro[2,3-d][1,3]dioxol-6(3aH)-one. MS: 458 (M+NH₄). ¹H NMR (300 MHz, Chloroform-d) δ 7.68-7.65 (m, 2H), 7.59-7.56 (m, 2H), 7.45-7.36 (m, 6H), 6.27 (d, J=4.5 Hz, 1H), 4.53 (d, J=4.5 Hz, 1H), 3.66-3.55 (m, 2H), 1.51 (s, 3H), 1.44 (s, 3H), 1.26 (s, 3H), 1.00 (s, 9H).

Step 5: To a stirred solution of (3aR,5R,6aS)-5-(((tert-butyldiphenylsilyl)oxy)methyl)-2,2,5-trimethyldihydrofuro[2,3-d][1,3]dioxol-6(3aH)-one (3 g, 6.8 mmol) in THF (25 mL) was added methyllithium (1.6 M in Et₂O, 10.6 mL, 17.0 mmol) dropwise at −78° C. The resulting mixture was stirred at −78° C. for 2 hours. The reaction mixture was quenched with saturated aqueous NH₄Cl (50 mL) and extracted with diethylether (100 mL×3). The combined organic layers were washed with brine (50 mL), dried over anhydrous sodium sulfate, and filtered. The filtrate was concentrated under reduced pressure, and the residue was purified by silica gel column chromatography (1-14% EtOAc/pet. ether) to afford (3aR,5R,6S,6aR)-5-(((tert-butyldiphenylsilyl)oxy)methyl)-2,2,5,6-tetramethyltetrahydrofuro[2,3-d][1,3]dioxol-6-ol. MS: 479 (M+Na). ¹H NMR (400 MHz, DMSO-d₆) δ 7.64-7.43 (m, 10H), 5.71 (d, J=4.4 Hz, 1H), 4.58 (s, 1H), 4.26 (d, J=4.4 Hz, 1H), 3.55-3.44 (m, 2H), 1.49 (s, 3H), 1.29 (s, 3H), 1.24 (s, 3H), 1.12 (s, 3H), 1.00 (s, 9H).

Step 6: To a solution of (3aR,5R,6S,6aR)-5-(((tert-butyldiphenylsilyl)oxy)methyl)-2,2,5,6-tetramethyltetrahydrofuro[2,3-d][1,3]dioxol-6-ol (2.3 g, 5.04 mmol) in 1,4-Dioxane (40 mL) and water (10 mL) was added 4-methylbenzenesulfonic acid (0.173 g, 1.01 mmol) at room temperature. The resulting mixture was stirred at 80° C. for 2 hours. The reaction mixture was cooled to room temperature and quenched with saturated aqueous NaHCO₃ (50 mL) and extracted with EtOAc (50 mL×3). The combined organic layers were washed with saturated aqueous NaHCO₃ (50 mL), dried over anhydrous sodium sulfate, and filtered. The filtrate was concentrated under reduced pressure, and the residue was purified by silica gel column chromatography (10-40% EtOAc/pet. ether) to afford (3R,4S,5R)-5-(((tert-butyldiphenylsilyl)oxy)methyl)-4,5-dimethyltetrahydrofuran-2,3,4-triol. MS: 439 (M+Na). ¹H NMR (400 MHz, DMSO-d₆) δ 7.75-7.39 (m, 10H), 5.65 (d, J=7.6 Hz, 1H), 5.04-5.01 (m, 1H), 4.66 (d, J=8.8 Hz, 1H), 4.35 (s, 1H), 4.06-4.01 (m, 2H), 3.40 (s, 1H), 1.22 (s, 3H), 1.04 (s, 3H), 0.98 (s, 9H).

Step 7: To a stirred solution of (3R,4S,5R)-5-(((tert-butyldiphenylsilyl)oxy)methyl)-4,5-dimethyltetrahydrofuran-2,3,4-triol (1.45 g, 3.5 mmol) (co-evaporated with dry MeCN 6 mL×3) in acetonitrile (20 mL) was added tributylphosphine (1.13 g, 5.6 mmol) and (E)-diazene-1,2-diylbis(piperidin-1-ylmethanone) (1.41 g, 5.6 mmol) at ambient temperature under an argon atmosphere. The reaction mixture was stirred for 1 h at room temperature. The resulting mixture containing (1S,3R,4S,5R)-3-(((tert-butyldiphenylsilyl)oxy)methyl)-3,4-dimethyl-2,6-dioxabicyclo[3.1.0]hexan-4-ol was used in the next step directly without work-up or purification.

Step 8: To a stirred solution of 4-chloro-7H-pyrrolo[2,3-d]pyrimidine (1.07 g, 6.96 mmol) in DMF (4 mL) was added NaH (418 mg, 10.4 mmol) at 0° C. The reaction mixture was stirred at room temperature for 0.5 h. A solution of (1S,3R,4S,5R)-3-(((tert-butyldiphenylsilyl)oxy)methyl)-3,4-dimethyl-2,6-dioxabicyclo[3.1.0]hexan-4-ol (˜3.5 mmol) in MeCN (20 mL) (from the previous step) was added to the above system at room temperature. The resulting mixture was stirred at room temperature for 0.5 h. The reaction mixture was quenched with saturated aqueous NaHCO₃ (35 mL) and extracted with EtOAc (50 mL×3). The combined organic layers were washed with water (100 mL) and brine (50 mL), dried over anhydrous sodium sulfate, and filtered. The filtrate was concentrated under reduced pressure, and the residue was purified by silica gel column chromatography (1-24% EtOAc/pet. ether) to afford (2R,3S,4R,5R)-2-(((tert-butyldiphenylsilyl)oxy)methyl)-5-(4-chloro-7H-pyrrolo[2,3-d]pyrimidin-7-yl)-2,3-dimethyltetrahydrofuran-3,4-diol. MS: 552 (M+1). ¹H NMR (300 MHz, DMSO-d6) δ 8.58 (s, 1H), 7.68-7.34 (m, 11H), 6.51 (d, J=3.6 Hz, 1H), 6.15 (d, J=8.4 Hz, 1H), 5.44 (d, J=7.2 Hz, 1H), 4.87 (s, 1H), 4.65-4.60 (m, 1H), 3.82 (d, J=10.8 Hz, 1H), 3.57 (d, J=11.1 Hz, 1H), 1.34 (s, 3H), 1.18 (s, 3H), 1.06 (s, 9H).

Step 9: To a solution of (2R,3S,4R,5R)-2-(((tert-butyldiphenylsilyl)oxy)methyl)-5-(4-chloro-7H-pyrrolo[2,3-d]pyrimidin-7-yl)-2,3-dimethyltetrahydrofuran-3,4-diol (600 mg, 1.09 mmol) in acetone (50 mL) were added 2,2-dimethoxypropane (1.13 g, 10.9 mmol) and 4-methylbenzenesulfonic acid (19 mg, 0.11 mmol) at room temperature. The reaction mixture was stirred at 40° C. for 5 h. The reaction mixture was quenched with saturated aqueous NaHCO₃ (40 mL), and the acetone was removed under reduced pressure. The aqueous phase was extracted with EtOAc (50 mL×3). The combined organic layers were washed with brine (50 mL), dried over anhydrous Na₂SO₄, and filtered. The filtrate was concentrated under reduced pressure, and the residue was purified by silica gel column chromatography (0-40% EtOAc/pet. ether) to afford 7-((3aR,4R,6R,6aS)-6-(((tert-butyldiphenylsilyl)oxy)methyl)-2,2,6,6a-tetramethyltetrahydrofuro[3,4-d][1,3]dioxol-4-yl)-4-chloro-7H-pyrrolo[2,3-d]pyrimidine. MS: 592 (M+1). ¹H NMR (400 MHz, CDCl₃) δ 8.66 (s, 1H), 7.65-7.53 (m, 5H), 7.55-7.45 (m, 1H), 7.49-7.28 (m, 5H), 6.48 (d, J=3.7 Hz, 1H), 6.30 (d, J=2.7 Hz, 1H), 5.15 (d, J=2.8 Hz, 1H), 3.64 (d, J=10.8 Hz, 1H), 3.54 (d, J=10.8 Hz, 1H), 1.66 (s, 3H), 1.53 (s, 6H), 1.46 (s, 3H), 1.08 (s, 9H).

Step 10: To a solution of 7-((3aR,4R,6R,6aS)-6-(((tert-butyldiphenylsilyl)oxy)methyl)-2,2,6,6a-tetramethyltetrahydrofuro[3,4-d][1,3]dioxol-4-yl)-4-chloro-7H-pyrrolo[2,3-d]pyrimidine (416 mg, 0.702 mmol) in tetrahydrofuran (5 mL) under an argon atmosphere was added tetrabutylammonium fluoride (1 M in THF, 2.11 mL, 2.11 mmol) at room temperature. The reaction solution was stirred at room temperature for 3 h. The solvent was removed under reduced pressure. The residue was purified by silica gel column chromatography (0-20% MeOH/DCM) to afford ((3aS,4R,6R,6aR)-6-(4-chloro-7H-pyrrolo[2,3-d]pyrimidin-7-yl)-2,2,3a,4-tetramethyltetrahydrofuro[3,4-d][1,3]dioxol-4-yl)methanol. MS: 354 (M+1). ¹H NMR (400 MHz, CDCl₃) δ 8.69 (s, 1H), 7.41 (d, J=3.7 Hz, 1H), 6.67 (d, J=3.7 Hz, 1H), 6.10 (d, J=5.1 Hz, 1H), 5.14 (d, J=5.1 Hz, 1H), 4.84 (dd, J=8.9, 3.8 Hz, 1H), 3.74-3.55 (m, 2H), 1.71 (s, 6H), 1.59 (s, 3H), 1.38 (s, 3H).

Step 11: To a solution of oxalyl dichloride (201 mg, 1.6 mmol) in anhydrous DCM (5 mL) was added DMSO (309 mg, 3.96 mmol) dropwise at −78° C. under an argon atmosphere. The resulting solution was stirred at −78° C. for 0.5 h. Then a solution of ((3aS,4R,6R,6aR)-6-(4-chloro-7H-pyrrolo[2,3-d]pyrimidin-7-yl)-2,2,3a,4-tetramethyltetrahydrofuro[3,4-d][1,3]dioxol-4-yl)methanol (140 mg, 0.40 mmol) in anhydrous DCM (5 mL) was added dropwise to the above reaction system at −78° C. The resulting solution was stirred at −78° C. for another 0.5 h. This was followed by the addition of TEA (400 mg, 4 mmol) at −78° C. The resulting solution was stirred for 0.5 h at −78° C. The reaction solution was quenched with H₂O (5 mL) at 0° C., and diluted with DCM (30 mL). The organic layer was washed with saturated aqueous NaHCO₃ (40 mL), brine (30 mL), dried over anhydrous Na₂SO₄, and filtered. The filtrate was concentrated under reduced pressure to afford (3aS,4S,6R,6aR)-6-(4-chloro-7H-pyrrolo[2,3-d]pyrimidin-7-yl)-2,2,3a,4-tetramethyltetrahydrofuro[3,4-d][1,3]dioxole-4-carbaldehyde, which was used in next step directly without further purification. MS: 352 (M+1). ¹H NMR (400 MHz, CDCl₃) δ 9.52 (s, 1H), 8.68 (s, 1H), 7.46 (d, J=3.7 Hz, 1H), 6.73 (d, J=3.7 Hz, 1H), 6.34 (d, J=2.3 Hz, 1H), 5.29 (d, J=2.3 Hz, 1H), 1.73 (s, 3H), 1.67 (s, 3H), 1.51 (s, 3H), 1.49 (s, 3H).

Step 12: To a solution of methyltriphenylphosphonium bromide (395 mg, 1.11 mmol) in anhydrous tetrahydrofuran (5 mL) was added n-BuLi (2.5 M in THF, 0.411 mL, 1.03 mmol) dropwise at −10° C. under an argon atmosphere. The reaction mixture was stirred at room temperature for 0.5 h. Then a solution of (3aS,4S,6R,6aR)-6-(4-chloro-7H-pyrrolo[2,3-d]pyrimidin-7-yl)-2,2,3a,4-tetramethyltetrahydrofuro[3,4-d][1,3]dioxole-4-carbaldehyde (139 mg, 0.396 mmol) in anhydrous tetrahydrofuran (8 mL) was added dropwise to the above reaction system at −10° C. The reaction mixture was stirred for 1 h at room temperature. The reaction mixture was quenched with saturated aqueous NH₄Cl (20 mL) at 0° C. The reaction solution was diluted with EtOAc (100 mL), washed with H₂O (20 mL) and brine (30 mL), dried over anhydrous Na₂SO₄, and filtered. The filtrate was concentrated under reduced pressure. The residue was purified by silica gel column chromatography (0-30% EtOAc/pet. ether) to afford 4-chloro-7-((3aR,4R,6R,6aS)-2,2,6,6a-tetramethyl-6-vinyltetrahydrofuro[3,4-d][1,3]dioxol-4-yl)-7H-pyrrolo[2,3-d]pyrimidine. MS: 350 (M+1). ¹H NMR (400 MHz, CDCl₃) δ 8.72 (s, 1H), 7.42 (d, J=3.7 Hz, 1H), 6.70 (d, J=3.7 Hz, 1H), 6.50 (d, J=2.5 Hz, 1H), 5.88 (dd, J=17.3, 11.0 Hz, 1H), 5.21 (dd, J=17.3, 1.3 Hz, 1H), 5.13 (dd, J=11.0, 1.3 Hz, 1H), 4.89 (d, J=2.5 Hz, 1H), 1.66 (s, 3H), 1.58 (s, 3H), 1.55 (s, 3H), 1.47 (s, 3H).

Step 13: To a sealed tube (20 mL) was added 4-chloro-7-((3aR,4R,6R,6aS)-2,2,6,6a-tetramethyl-6-vinyltetrahydrofuro[3,4-d][1,3]dioxol-4-yl)-7H-pyrrolo[2,3-d]pyrimidine (50 mg, 0.143 mmol), 1,4-dioxane (8 mL) and NH₃H₂O (8 mL, 25%-28% wt) at room temperature. The mixture was sealed tightly and then stirred at 90° C. for 16 hours. The reaction mixture was cooled to room temperature and concentrated under reduced pressure. The residue was purified by silica gel column chromatography (0-10% MeOH/DCM) to afford 7-((3aR,4R,6R,6aS)-2,2,6,6a-tetramethyl-6-vinyltetrahydrofuro[3,4-d][1,3]dioxol-4-yl)-7H-pyrrolo[2,3-d]pyrimidin-4-amine. MS: 331 (M+1). ¹H NMR (400 MHz, CDCl₃) δ 8.38 (s, 1H), 7.15 (d, J=3.7 Hz, 1H), 6.49 (d, J=2.6 Hz, 1H), 6.46 (d, J=3.7 Hz, 1H), 5.89 (dd, J=17.3, 11.0 Hz, 1H), 5.33 (s, 2H), 5.23 (dd, J=17.3, 1.4 Hz, 1H), 5.12 (dd, J=11.0, 1.4 Hz, 1H), 4.82 (d, J=2.5 Hz, 1H), 1.65 (s, 3H), 1.57 (s, 3H), 1.54 (s, 3H), 1.45 (s, 3H).

Step 14: To a solution of 7-((3aR,4R,6R,6aS)-2,2,6,6a-tetramethyl-6-vinyltetrahydrofuro[3,4-d][1,3]dioxol-4-yl)-7H-pyrrolo[2,3-d]pyrimidin-4-amine (20 mg, 0.061 mmol) in anhydrous THF (1.0 mL) was added 9-BBN in THF (0.605 mL, 0.303 mmol, 0.5M) dropwise at 0° C. under an argon atmosphere. The reaction solution was stirred at 50° C. for 1 h. The resulting solution was used in next step directly.

Step 15: To the above solution was added potassium phosphate tribasic (64.3 mg, 0.303 mmol) in water (0.2 mL) dropwise at 0° C. under an atmosphere of argon. The reaction solution was stirred at room temperature for 0.5 h. 3-bromo-7-iodo-N-(4-methoxybenzyl)quinolin-2-amine (31.3 mg, 0.067 mmol) in tetrahydrofuran (0.3 mL) and 1,1′-bis(diphenylphosphino)ferrocene-palladium (II) dichloride dichloromethane complex (4.95 mg, 6.06 μmol) were added to the above reaction system respectively at room temperature. The reaction mixture was heated at 70° C. for 2 h under microwave irradiation. The reaction mixture was then cooled to room temperature, diluted with water (5 mL) and extracted with EtOAc (2×20 mL). The combined organic layers were dried over anhydrous sodium sulfate and filtered. The filtrate was concentrated under reduced pressure. The residue was purified by Prep-TLC (developed by 10% MeOH in DCM) to afford 7-(2-((3aS,4R,6R,6aR)-6-(4-amino-7H-pyrrolo[2,3-d]pyrimidin-7-yl)-2,2,3a,4-tetramethyltetrahydrofuro[3,4-d][1,3]dioxol-4-yl)ethyl)-3-bromo-N-(4-methoxybenzyl)quinolin-2-amine. MS: 673/675 (M+1/M+3). ¹H NMR (400 MHz, CDCl₃) δ 8.35 (s, 1H), 8.05 (s, 1H), 7.43-7.29 (m, 4H), 7.19 (d, J=3.7 Hz, 1H), 6.93 (d, J=8.6 Hz, 2H), 6.74 (d, J=8.1 Hz, 1H), 6.47 (d, J=3.7 Hz, 1H), 6.34 (d, J=2.4 Hz, 1H), 5.65-5.54 (m, 1H), 5.40-5.29 (m, 2H), 4.77 (d, J=5.0 Hz, 2H), 3.84 (s, 3H), 2.76-2.68 (m, 1H), 2.55-2.45 (m, 1H), 2.05-1.93 (m, 1H), 1.87-1.76 (m, 1H), 1.66 (s, 3H), 1.63 (s, 3H), 1.54 (s, 3H), 1.51 (s, 3H).

Step 16: A solution of 7-(2-((3aS,4R,6R,6aR)-6-(4-amino-7H-pyrrolo[2,3-d]pyrimidin-7-yl)-2,2,3a,4-tetramethyltetrahydrofuro[3,4-d][1,3]dioxol-4-yl)ethyl)-3-bromo-N-(4-methoxybenzyl)quinolin-2-amine (40 mg, 0.059 mmol) in TFA (2 mL, 26.0 mmol) was stirred at 60° C. for 1 h. The reaction was cooled to room temperature and concentrated under reduced pressure to afford the crude product N-(7-(2-((2R,3S,4R,5R)-5-(4-amino-7H-pyrrolo[2,3-d]pyrimidin-7-yl)-3,4-dihydroxy-2,3-dimethyltetrahydrofuran-2-yl)ethyl)-3-bromoquinolin-2-yl)-2,2,2-trifluoroacetamide, which was used in next step directly without further purification.

Step 17: To a solution of the crude N-(7-(2-((2R,3S,4R,5R)-5-(4-amino-7H-pyrrolo[2,3-d]pyrimidin-7-yl)-3,4-dihy droxy-2,3-dimethyltetrahydrofuran-2-yl)ethyl)-3-bromoquinolin-2-yl)-2,2,2-trifluoroacetamide (calculated as 0.059 mmol) in methanol (3 mL) was added K₂CO₃ (24.6 mg, 0.178 mmol) at room temperature. The reaction mixture was stirred at 60° C. for 1 h. The solid was filtered and washed with MeOH (0.5 mL). The filtrate was concentrated under reduced pressure, and the residue was purified by reverse phase column chromatography (ACN/water with 5 mM NH₄HCO₃ modifier) to afford (2R,3S,4R,5R)-2-(2-(2-amino-3-bromoquinolin-7-yl)ethyl)-5-(4-amino-7H-pyrrolo[2,3-d]pyrimidin-7-yl)-2,3-dimethyltetrahydrofuran-3,4-diol as solid. MS: 513/515 (M+1/M+3). ¹H NMR (400 MHz, DMSO-d₆) δ 8.33 (s, 1H), 8.06 (s, 1H), 7.58 (d, J=8.0 Hz, 1H), 7.38 (d, J=4.0 Hz, 1H), 7.30 (s, 1H), 7.08 (d, J=8.4 Hz, 1H), 7.01 (br s, 2H), 6.64 (d, J=3.6 Hz, 1H), 6.58 (br s, 2H), 6.03 (d, J=8.0 Hz, 1H), 5.29 (br s, 1H), 4.79-4.77 (m, 1H), 4.72 (s, 1H), 2.66-2.59 (m, 2H), 2.20-2.15 (m, 1H), 1.70-1.63 (m, 1H), 1.25 (s, 3H), 1.17 (s, 3H).

### Example 5

### (2R,3R,3aS,6S,6aR)-6-((2-amino-3-bromoquinolin-7-yl)oxy)-2-(4-chloro-7H-pyrrolo[2,3-d]pyrimidin-7-yl)hexahydro-2H-cyclopenta[b]furan-3,3a-diol

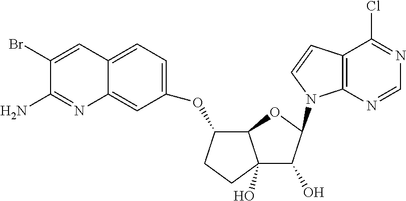 [SMILES: Nc1nc2cc(O[C@H]3CC[C@@]4(O)[C@@H]3O[C@@H](n3ccc5c(Cl)ncnc53)[C@@H]4O)ccc2cc1Br]

Step 1: (3R,3aS,6R,6aR)-2-methoxyhexahydro-2H-cyclopenta[b]furan-3,3a,6-triol (2 g, 10 mmol) was co-evaporated with dry toluene (5 mL×3) and then re-dissolved in acetone (50 mL). To this solution was added 4-methylbenzenesulfonic acid (0.091 g, 0.53 mmol), followed by 2,2-dimethoxypropane (2.74 g, 26.3 mmol). The resulting mixture was stirred at ambient temperature for 1 h. The pH of the resulting solution was adjusted to 8 with saturated aqueous NaHCO₃ (50 mL) at 0° C. The resulting mixture was extracted with EtOAc (150 mL×3). The combined organic layers were washed with brine (100 mL), dried over anhydrous Na₂SO₄, filtered, and the filtrate was concentrated under reduced pressure. The residue was purified by column chromatography on silica (EtOAc/pet. ether) to afford (3aR,5aR,6R,8aR)-4-methoxy-2,2-dimethylhexahydrocyclopenta[2,3]furo[3,4-d][1,3]dioxol-6-ol. MS: 248.20 (M+NH₄). ¹H NMR (300 MHz, DMSO-d₆) δ 4.96 (s, 1H), 4.41 (d, J=5.1 Hz, 1H), 4.17 (s, 1H), 4.10 (d, J=6.0 Hz, 1H), 3.88-3.79 (m, 1H), 3.33 (s, 3H), 2.04-1.92 (m, 1H), 1.76-1.62 (m, 3H), 1.39 (s, 3H), 1.31 (s, 3H). The column was further eluted with 45-50% of EtOAc in petroleum ether to afford (3aR,5aR,6R,8aR)-4-methoxy-2,2-dimethylhexahydrocyclopenta[2,3]furo[3,4-d][1,3]dioxol-6-ol. MS: 248 (M+NH₄). ¹H NMR (300 MHz, DMSO-d₆) δ 4.92 (d, J=4.2 Hz, 1H), 4.72 (d, J=6.0 Hz, 1H), 4.35 (d, J=4.2 Hz, 1H), 4.00 (d, J=(5.4 Hz, 1H), 3.91-3.82 (m, 1H), 3.35 (s, 3H), 2.09-1.97 (m, 1H), 1.83-1.62 (m, 2H), 1.52-1.43 (m, 1H), 1.40 (s, 3H), 1.31 (s, 3H).

Step 2: To a solution of (3aR,5aR,6R,8aR)-4-methoxy-2,2-dimethylhexahydrocyclopenta[2,3]furo[3,4-d][1,3]dioxol-6-ol (748 mg, 3.25 mmol) in DCM (30 mL) was added N,N-dimethylpyridin-4-amine (437 mg, 3.57 mmol) at room temperature. Then triethylamine (362 mg, 3.57 mmol) was added, followed by 4-methylbenzene-1-sulfonyl chloride (929 mg, 4.87 mmol). The reaction mixture was stirred for 16 h at 25° C. The resulting mixture was quenched with saturated aqueous NH₄Cl (200 mL) and extracted with DCM (100 mL×3). The combined organic layers were washed with brine (200 mL) and dried over anhydrous sodium sulfate. After filtration, the filtrate was concentrated under reduced pressure, and the residue was purified by column chromatography on silica (EtOAc/pet. ether) to afford (3aR,5aR,6R,8aR)-4-methoxy-2,2-dimethylhexahydrocyclopenta[2,3]furo[3,4-d][1,3]dioxol-6-yl 4-methylbenzenesulfonate. MS: 402 (M+NH₄). ¹H NMR (300 MHz, Chloroform-d) δ 7.85-7.81 (m, 2H), 7.35-7.32 (m, 2H), 4.94 (d, J=4.2 Hz, 1H), 4.74-4.67 (m, 1H), 4.35 (d, J=4.2 Hz, 1H), 4.15 (d, J=5.1 Hz, 1H), 3.38 (s, 3H), 2.44 (s, 3H), 2.18-1.75 (m, 4H), 1.49 (s, 3H), 1.37 (s, 3H).

Step 3: To a solution of (3aR,5aR,6R,8aR)-4-methoxy-2,2-dimethylhexahydrocyclopenta[2,3]furo[3,4-d][1,3]dioxol-6-yl 4-methylbenzenesulfonate (400 mg, 1.04 mmol) and 2-amino-3-bromoquinolin-7-ol (249 mg, 1.04 mmol) (azeotroped with toluene 2 mL×3) in NMP (5 mL) was added Cs₂CO₃ (1.02 g, 3.12 mmol). The reaction mixture was stirred at 90° C. for 2 h under an argon atmosphere. The reaction mixture was cooled to room temperature and quenched with saturated aqueous NH₄Cl (60 mL) and extracted with DCM (60 mL×3). The combined organic layers were washed with brine (60 mL) and dried over anhydrous Na₂SO₄. After filtration, the filtrate was concentrated under reduced pressure. The residue was purified by column chromatography on silica (EtOAc/pet. Ether). The product was further purified by reverse phase HPLC (ACN/water) to afford 3-bromo-7-(((3aR,5aR,6S,8aR)-4-methoxy-2,2-dimethylhexahydrocyclopenta[2,3]furo[3,4-d][1,3]dioxol-6-yl)oxy)quinolin-2-amine. MS: 451/453 (M+1/M+3). ¹H NMR (300 MHz, Chloroform-d) δ 8.06 (s, 1H), 7.46 (d, J=9.0 Hz, 1H), 7.06 (d, J=2.4 Hz, 1H), 6.95 (dd, J=8.7, 2.4 Hz, 1H), 5.28 (s, 2H), 4.93 (d, J=4.2 Hz, 1H), 4.66 (d, J=4.2 Hz, 1H), 4.63-4.50 (m, 2H), 3.49 (s, 3H), 2.45-2.33 (m, 1H), 2.26-2.14 (m, 2H), 2.01-1.88 (m, 1H), 1.55 (s, 3H), 1.44 (s, 3H).

Step 4: A solution of 3-bromo-7-(((3aR,5aR,6S,8aR)-4-methoxy-2,2-dimethylhexahydrocyclopenta[2,3]furo[3,4-d][1,3]dioxol-6-yl)oxy)quinolin-2-amine (315 mg, 0.698 mmol) in HCl (10 mL, 4.00 mmol, 0.4 M in MeCN/H₂O=3:2 (v/v)) was stirred at 90° C. for 2 h. The reaction mixture was quenched with saturated aqueous NaHCO₃ (50 mL) and extracted with DCM (50 mL×3). The combined organic layers were washed with brine (50 mL), dried over anhydrous Na₂SO₄, and filtered. The filtrate was concentrated under reduced pressure. The combined residue was purified by reverse phase HPLC (ACN/water) to afford (3R,3aS,6S,6aR)-6-((2-amino-3-bromoquinolin-7-yl)oxy)hexahydro-2H-cyclopenta[b]furan-2,3,3a-triol. MS: 397/399 (M+1/M+3). ¹H NMR (300 MHz, Methanol-d4) δ 8.20 (s, 1H), 7.56-7.52 (m, 1H), 7.11 (d, J=2.4 Hz, 1H), 7.02 (d, J=2.4 Hz, 1H), 6.97-6.92 (m, 1H), 5.34 (d, J=4.2 Hz, 1H), 5.20 (d, J=3.0 Hz, 1H), 4.76-4.74 (m, 1H), 4.64-4.62 (m, 1H), 4.36 (s, 1H), 4.18 (s, 1H), 3.79 (d, J=4.2 Hz, 1H), 3.63 (d, J=3.3 Hz, 1H), 2.46-1.81 (m, 4H).

Step 5: To a stirred solution of (3R,3aS,6S,6aR)-6-((2-amino-3-bromoquinolin-7-yl)oxy)hexahydro-2H-cyclopenta[b]furan-2,3,3a-triol (240 mg, 0.604 mmol) in anhydrous MeCN (10 mL) was added tributylphosphine (0.241 mL, 0.967 mmol), followed by (E)-diazene-1,2-diylbis(piperidin-1-ylmethanone) (229 mg, 0.906 mmol) at room temperature. The reaction mixture was stirred at ambient temperature for 20 minutes, and the solution containing crude (1aS,2aR,3S,5aR,5bR)-3-((2-amino-3-bromoquinolin-7-yl)oxy)hexahydro-5aH-cyclopenta[b]oxireno[2,3-d]furan-5a-ol was concentrated under reduced pressure. The product was used in next step without purification.

Step 6: To a stirred solution of 4-chloro-7H-pyrrolo[2,3-d]pyrimidine (184 mg, 1.20 mmol) in dry DMF (4 mL) was added sodium hydride (72 mg, 1.8 mmol) at 0° C. The suspension was stirred at room temperature for 30 minutes, then the suspension was transferred to a solution containing crude (1aS,2aR,3S,5aR,5bR)-3-((2-amino-3-bromoquinolin-7-yl)oxy)hexahydro-5aH-cyclopenta[b]oxireno[2,3-d]furan-5a-ol in ACN (4 mL) via syringe. The resulting mixture was stirred at room temperature for 2 h. The reaction mixture was quenched with saturated aqueous NH₄Cl (50 mL) and extracted with DCM (50 mL×3). The combined organic layers were washed with brine (50 mL), dried over anhydrous Na₂SO₄, and filtered. The filtrate was concentrated under reduced pressure, and the residue was purified by reverse phase HPLC (ACN/water with 5 mM aqueous ammonium bicarbonate). The crude product was further purified by Prep-TLC, developed by DCM:MeOH=10:1 (v:v) (rf=0.6) to afford (2R,3R,3aS,6S,6aR)-6-((2-amino-3-bromoquinolin-7-yl)oxy)-2-(4-chloro-7H-pyrrolo[2,3-d]pyrimidin-7-yl)hexahydro-2H-cyclopenta[b]furan-3,3a-diol. MS: 532/534 (M+1/M+3). ¹H NMR (300 MHz, DMSO-d₆) δ 8.72 (s, 1H), 8.27 (s, 1H), 8.18 (d, J=3.9 Hz, 1H), 7.57 (d, J=8.7 Hz, 1H), 6.88-6.83 (m, 3H), 6.61 (br s, 2H), 6.17 (d, J=8.4 Hz, 1H), 5.53 (d, J=7.2 Hz, 1H), 5.44 (s, 1H), 4.67 (d, J=4.8 Hz, 1H), 4.48 (t, J=7.8 Hz, 1H), 4.15 (s, 1H), 2.51-2.50 (m, 1H), 2.10-1.95 (m, 3H).

### Example 6

### (2R,3R,3aS,6S,6aR)-6-((2-amino-3-bromoquinolin-7-yl)oxy)-2-(4-amino-7H-pyrrolo[2,3-d]pyrimidin-7-yl)hexahydro-2H-cyclopenta[b]furan-3,3a-diol

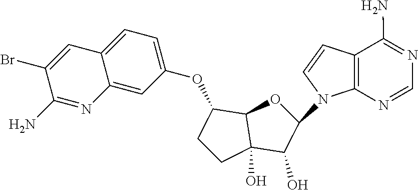 [SMILES: Nc1nc2cc(O[C@H]3CC[C@@]4(O)[C@@H]3O[C@@H](n3ccc5c(N)ncnc53)[C@@H]4O)ccc2cc1Br]

Step 1: A solution of (2R,3R,3aS,6S,6aR)-6-((2-amino-3-bromoquinolin-7-yl)oxy)-2-(4-chloro-7H-pyrrolo[2,3-d]pyrimidin-7-yl)hexahydro-2H-cyclopenta[b]furan-3,3a-diol (55 mg, 0.10 mmol), 1,4-dioxane (15 mL) and NH₃—H₂O (15 mL) was stirred in a sealed tube at 90° C. for 10 h. The reaction mixture was concentrated under reduced pressure, and the residue was diluted with saturated aqueous NH₄Cl (30 mL). The mixture was extracted with DCM (25 mL×5). The combined organic layers were washed with brine (50 mL), dried over anhydrous sodium sulfate, and filtered. The filtrate was concentrated under reduced pressure, and the residue was purified by column chromatography on silica (MeOH/DCM) to afford (2R,3R,3aS,6S,6aR)-6-((2-amino-3-bromoquinolin-7-yl)oxy)-2-(4-amino-7H-pyrrolo[2,3-d]pyrimidin-7-yl)hexahydro-2H-cyclopenta[b]furan-3,3a-diol. MS: 513/515 (M+1/M+3). ¹H NMR (400 MHz, DMSO-d₆) δ 8.26 (s, 1H), 8.09 (s, 1H), 7.57 (d, J=8.8 Hz, 1H), 7.50 (d, J=3.6 Hz, 1H), 7.05 (br s, 2H), 6.87-6.83 (m, 2H), 6.66 (d, J=4.0 Hz, 1H), 6.53 (br s, 2H), 6.02 (d, J=8.8 Hz, 1H), 5.36 (d, J=7.2 Hz, 1H), 5.31 (s, 1H), 4.60 (d, J=4.8 Hz, 1H), 4.40 (t, J=7.6 Hz, 1H), 4.08 (s, 1H), 2.52-2.50 (m, 1H), 2.07-1.99 (m, 3H).

### Example 7

### (2R,3R,3aS,6S,6aR)-6-((2-amino-3-bromoquinolin-7-yl)oxy)-2-(4-methyl-7H-pyrrolo[2,3-d]pyrimidin-7-yl)hexahydro-2H-cyclopenta[b]furan-3,3a-diol

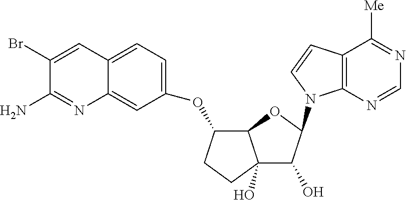 [SMILES: Cc1ncnc2c1ccn2[C@@H]1O[C@@H]2[C@@H](Oc3ccc4cc(Br)c(N)nc4c3)CC[C@]2(O)[C@H]1O]

Step 1: To a stirred solution of (3R,3aS,6S,6aR)-6-((2-amino-3-bromoquinolin-7-yl)oxy)hexahydro-2H-cyclopenta[b]furan-2,3,3a-triol (1.0 g, 2.5 mmol) in dry MeCN (30 mL) under an argon atmosphere was added (E)-diazene-1,2-diylbis(piperidin-1-ylmethanone) (1.13 g, 4.53 mmol) and tributylphosphine (1.20 mL, 4.78 mmol) at 25° C. The resulting mixture was stirred at 25° C. for 40 minutes. This solution was used in the next step without isolation and characterization.

Step 2: To a stirred solution of 4-methyl-7H-pyrrolo[2,3-d]pyrimidine (0.671 g, 5.04 mmol) (co-evaporated with dry toluene 10 mL×3 before being used) in anhydrous DMF (12 mL) was added sodium hydride (0.302 g, 7.56 mmol) at 0° C. The suspension was stirred at room temperature for 30 minutes. The suspension was transferred via a syringe at ambient temperature into the solution from the previous step, which contained (1aS,2aR,3S,5aR,5bR)-3-((2-amino-3-bromoquinolin-7-yl)oxy)hexahydro-1aH-cyclopenta[b]oxireno[2,3-d]furan-5a-ol (calculated as ˜2.52 mmol). The resulting mixture was stirred at room temperature for 30 minutes. The reaction was quenched with saturated aqueous NH₄Cl (40 mL) and concentrated under reduced pressure. The residue was purified by reverse-phase HPLC (0-100% acetonitrile/water with 5 mM ammonium bicarbonate modifier) to afford (2R,3R,3aS,6S,6aR)-6-((2-amino-3-bromoquinolin-7-yl)oxy)-2-(4-methyl-7H-pyrrolo[2,3-d]pyrimidin-7-yl)hexahydro-2H-cyclopenta[b]furan-3,3a-diol as a solid. MS: 512/514 (M+1/M+3). ¹H NMR (400 MHz, DMSO-d₆) δ 8.70 (s, 1H), 8.26 (s, 1H), 7.95 (d, J=4.0 Hz, 1H), 7.57 (d, J=8.8 Hz, 1H), 6.88-6.83 (m, 3H), 6.52 (br s, 2H), 6.15 (d, J=8.4 Hz, 1H), 5.45 (d, J=7.2 Hz, 1H), 5.39 (s, 1H), 4.64 (d, J=4.8 Hz, 1H), 4.48 (t, J=8.0 Hz, 1H), 4.14 (s, 1H), 2.69 (s, 3H), 2.56-2.50 (m, 1H), 2.10-1.99 (m, 3H).

### Example 8

### (2R,3R,3aS,6S,6aR)-6-((2-amino-3-bromoquinolin-7-yl)methyl)-2-(4-methyl-7H-pyrrolo[2,3-d]pyrimidin-7-yl)hexahydro-2H-cyclopenta[b]furan-3,3a-diol

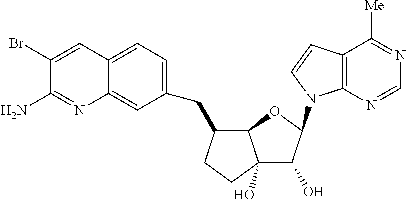

Step 1: To a solution of oxalyl dichloride (1.47 mL, 17.4 mmol) in anhydrous DCM (20 mL) was added DMSO (3.08 mL, 43.4 mmol) in anhydrous DCM (2 mL) at −78° C. under argon atmosphere. The reaction was stirred at −65° C. for 0.5 h. A solution of (3aR,5aR,6R,8aR)-4-methoxy-2,2-dimethylhexahydrocyclopenta[2,3]furo[3,4-d][1,3]dioxol-6-ol (1.0 g, 4.34 mmol) in anhydrous DCM (10 mL) was added to the above solution at −65° C. The solution was stirred for another 0.5 h at −65° C. Under this temperature, TEA (6.05 mL, 43.4 mmol) was added to the reaction mixture. The resulting solution was stirred for 0.5 h at −65° C. The reaction was quenched with H₂O (50 mL) at 0° C. and extracted with DCM (100 mL×3). The combined organic layers were washed with saturated aqueous NaHCO₃ (40 mL) and brine (30 mL), dried over anhydrous Na₂SO₄, and filtered. The filtrate was concentrated under reduced pressure to give (3aR,5aS,8aS)-4-methoxy-2,2-dimethyltetrahydrocyclopenta[2,3]furo[3,4-d][1,3]dioxol-6(5aH)-one, which was azeotroped with toluene (10 mL×3) and used directly in the next step without purification. ¹H NMR (400 MHz, DMSO-d₆) δ 5.77 (d, J=1.6 Hz, 1H), 4.98 (d, J=1.6 Hz, 1H), 4.40 (d, J=1.6 Hz, 1H), 4.16 (s, 1H), 3.10 (s, 3H), 2.50-2.30 (m, 3H), 1.39 (s, 3H), 1.37 (s, 3H).

Step 2: To a solution of methyltriphenylphosphonium bromide (4.34 g, 12.2 mmol) in anhydrous THF (20 mL) was added n-BuLi (2.5 M in THF) (4.52 mL, 11.3 mmol) dropwise at −60° C. under an argon atmosphere. The reaction mixture was stirred at room temperature for 0.5 h. Then a solution of (3aR,5aS,8aS)-4-methoxy-2,2-dimethyltetrahydrocyclopenta[2,3]furo[3,4-d][1,3]dioxol-6(5aH)-one (1.8 g crude, 4.3 mmol) in anhydrous THF (20 mL) was added dropwise at −60° C. The reaction mixture was stirred at room temperature for 1 h. The reaction was quenched with saturated aqueous NH₄Cl (50 mL) at 0° C. The mixture was extracted with EtOAc (200 mL×2), and the combined organic layers were washed with brine (50 mL), dried over anhydrous Na₂SO₄, and filtered. The filtrate was concentrated under reduced pressure. The residue was purified by silica gel column chromatography (EtOAc/pet. ether) to afford (3aR,5aR,8aR)-4-methoxy-2,2-dimethyl-6-methylenehexahydrocyclopenta[2,3]furo[3,4-d][1,3]dioxole. ¹H NMR (400 MHz, Chloroform-d) δ 5.17 (s, 1H), 5.07 (s, 1H), 5.02 (t, J=1.1 Hz, 1H), 4.64 (s, 1H), 4.30 (t, J=1.1 Hz, 1H), 3.29 (s, 3H), 2.75-2.59 (m, 1H), 2.44-2.33 (m, 1H), 2.23-2.09 (m, 1H), 2.15-2.05 (m, 1H), 1.51 (s, 3H), 1.41 (s, 3H).

Step 3: To a solution of (3aR,5aR,8aR)-4-methoxy-2,2-dimethyl-6-methylenehexahydrocyclopenta[2,3]furo[3,4-d][1,3]dioxole (300 mg, 1 mmol) in anhydrous THF (3 mL) was added 9-BBN (0.5 M in THF, 13.3 mL, 6.63 mmol) dropwise at 0° C. under an argon atmosphere. The reaction mixture was stirred at 50° C. for 1 h. This solution was used in the next step without characterization.

Step 4: To the solution containing the borane intermediate from the previous step was added a solution of potassium phosphate tribasic (1.41 g, 6.63 mmol) in water (3 mL) at 0° C. under an argon atmosphere. The reaction mixture was stirred at room temperature for 0.5 h. A solution of 3-bromo-7-iodo-N-(4-methoxybenzyl)quinolin-2-amine (684 mg, 1.46 mmol) and 1,1′-bis(diphenylphosphino)ferrocene-palladium(II)dichloridedichhloromethane complex (108 mg, 0.133 mmol) in THF (5 mL) was added at room temperature. The reaction mixture was stirred at 50° C. for 1.5 h. The reaction mixture was cooled to room temperature and partitioned between brine (80 mL) and EtOAc (100 mL). The aqueous phase was back-extracted with EtOAc (100 mL×2). The combined organic layers were dried over anhydrous sodium sulfate, filtered, and the filtrate was concentrated under reduced pressure. The residue was purified by column chromatography on silica (EtOAc/pet. ether) to afford 3-bromo-7-(((3aR,5aR,8aR)-4-methoxy-2,2-dimethylhexahydrocyclopenta[2,3]furo[3,4-d][1,3]dioxol-6-yl)methyl)-N-(4-methoxybenzyl)quinolin-2-amine. The crude product was used in next step directly without further purification. MS: 569/571 (M+1/M+3).

Step 5: A solution of 3-bromo-7-(((3aR,5aR,8aR)-4-methoxy-2,2-dimethylhexahydrocyclopenta[2,3]furo[3,4-d][1,3]dioxol-6-yl)methyl)-N-(4-methoxybenzyl)quinolin-2-amine (890 mg crude,1.33 mmol) in HCl (0.4 M in MeCN/H₂O (3:2, v/v), 10 mL, 4 mmol) was stirred at 90° C. for 1 h. The reaction was cooled to 0° C., quenched with saturated aqueous Na₂CO₃ (60 mL), and extracted with EtOAc (100 mL×3). The combined organic layers were washed with brine (60 mL), dried over anhydrous sodium sulfate, filtered, and the filtrate was concentrated under reduced pressure. The residue was purified by silica gel column chromatography (MeOH/DCM) to afford (3R,3aS,6aR)-6-((3-bromo-2-((4-methoxybenzyl)amino)quinolin-7-yl)methyl)hexahydro-2H-cyclopenta[b]furan-2,3,3a-triol. MS: 515/517 (M+1/M+3). ¹H NMR (400 MHz, DMSO-d₆) δ 8.33 (s, 1H), 7.57 (d, J=8.3 Hz, 1H), 7.35 (d, J=8.4 Hz, 3H), 7.13 (d, J=7.5 Hz, 2H), 6.88 (d, J=8.2 Hz, 2H), 5.93 (d, J=6.5 Hz, 1H), 5.20-5.12 (m, 1H), 4.64 (t, J=8.1 Hz, 3H), 4.46 (d, J=8.1 Hz, 1H), 3.93 (d, J=4.7 Hz, 1H), 3.72 (d, J=1.1 Hz, 3H), 3.49 (dd, J=7.5, 4.1 Hz, 1H), 2.87-2.76 (m, 1H), 2.65 (dd, J=13.6, 7.0 Hz, 1H), 2.20-1.19 (m, 5H).

Step 6: To a stirred solution of (3R,3aS,6aR)-6-((3-bromo-2-((4-methoxybenzyl)amino)quinolin-7-yl)methyl)hexahydro-2H-cyclopenta[b]furan-2,3,3a-triol (250 mg, 0.486 mmol) in dry MeCN (9 mL) was added tributylphosphine (176 mg, 0.869 mmol), followed by (E)-diazene-1,2-diylbis(piperidin-1-ylmethanone) (206 mg, 0.815 mmol) at room temperature. The reaction mixture was stirred at room temperature for 1 h, and the solution was used directly in the next step without characterization.

Step 7: To a stirred solution of 4-methyl-7H-pyrrolo[2,3-d]pyrimidine (129 mg, 0.970 mmol) in dry DMF (6 mL) was added sodium hydride (60% dispersion in mineral oil) (58.2 mg, 1.46 mmol) at 0° C. The suspension was stirred at room temperature for 30 minutes. The suspension was transferred to the solution from the previous step containing the epoxide intermediate via syringe, and the resulting mixture was stirred at room temperature for 2 h. The reaction mixture was quenched with saturated aqueous ammonium chloride (30 mL) and extracted with EtOAc (40 mL×3). The combined organic layers were washed with brine (40 mL), dried over anhydrous sodium sulfate, and filtered. The filtrate was concentrated under reduced pressure. The residue was purified by Preparative TLC (MeOH/DCM) to afford (2R,3R,3aS,6aR)-6-((3-bromo-2-((4-methoxybenzyl)amino)quinolin-7-yl)methyl)-2-(4-methyl-7H-pyrrolo[2,3-d]pyrimidin-7-yl)hexahydro-3aH-cyclopenta[b]furan-3,3a-diol. MS: 630/632 (M+1/M+3). ¹H NMR (400 MHz, DMSO-d₆) δ 8.69 (s, 1H), 8.32 (d, J=6.0 Hz, 1H), 8.02 (s, 1H), 7.54 (d, J=8.2 Hz, 1H), 7.33 (d, J=8.4 Hz, 2H), 7.14-7.04 (m, 3H), 6.91-6.80 (m, 4H), 6.03 (d, J=8.1 Hz, 1H), 5.30 (d, J=7.0 Hz, 1H), 5.12 (s, 1H), 4.61 (d, J=6.2 Hz, 2H), 4.22 (t, J=7.6 Hz, 1H), 4.04 (d, J=6.6 Hz, 1H), 3.72 (s, 3H), 2.83 (dd, J=13.7, 7.2 Hz, 1H), 2.69 (s, 3H), 2.65 (s, 1H), 2.37-2.22 (m, 1H), 1.99-1.93 (m, 1H), 1.55 (d, J=6.5 Hz, 2H).

Step 8: A solution of (2R,3R,3aS,6aR)-6-((3-bromo-2-((4-methoxybenzyl)amino)quinolin-7-yl)methyl)-2-(4-methyl-7H-pyrrolo[2,3-d]pyrimidin-7-yl)hexahydro-3aH-cyclopenta[b]furan-3,3a-diol (150 mg, 0.21 mmol) in TFA (2 mL) was stirred at 60° C. for 1 h. The reaction mixture was quenched with saturated aqueous NaHCO₃ (50 mL) and extracted with EtOAc (50 mL×3). The combined organic layers were washed with brine (50 mL), dried over anhydrous sodium sulfate, and filtered. The filtrate was concentrated under reduced pressure. The residue was purified by preparative TLC on silica (MeOH/DCM) to afford crude product as a solid. The crude product was further purified by reverse phase HPLC (ACN/water with 5 mM ammonium bicarbonate modifier) to afford (2R,3R,3aS,6S,6aR)-6-((2-amino-3-bromoquinolin-7-yl)methyl)-2-(4-methyl-7H-pyrrolo[2,3-d]pyrimidin-7-yl)hexahydro-2H-cyclopenta[b]furan-3,3a-diol. MS: 510/512 (M+1/M+3). ¹H NMR (400 MHz, DMSO-d₆) δ 8.69 (s, 1H), 8.30 (s, 1H), 7.87 (d, J=3.6 Hz, 1H), 7.54 (d, J=8.4 Hz, 1H), 7.27 (s, 1H), 7.08 (dd, J=8.4, 1.6 Hz, 1H), 6.82 (d, J=3.6 Hz, 1H), 6.53 (br s, 2H), 6.01 (d, J=8.0 Hz, 1H), 5.30 (d, J=6.8 Hz, 1H), 5.11 (s, 1H), 4.22 (t, J=7.6 Hz, 1H), 4.00 (d, J=5.6 Hz, 1H), 2.83 (dd, J=13.6, 7.8 Hz, 1H), 2.70 (s, 3H), 2.64 (dd, J=13.6, 7.6 Hz, 1H), 2.35-2.24 (m, 1H), 1.97 (dd, J=12.4, 5.6 Hz, 1H), 1.79-1.68 (m, 2H), 1.55 (dt, J=12.4, 6.4 Hz, 1H).

### Example 9

### (2R,3R,3aS,6S,6aR)-6-((2-amino-3-bromoquinolin-7-yl)methyl)-2-(4-amino-7H-pyrrolo[2,3-d]pyrimidin-7-yl)hexahydro-2H-cyclopenta[b]furan-3,3a-diol

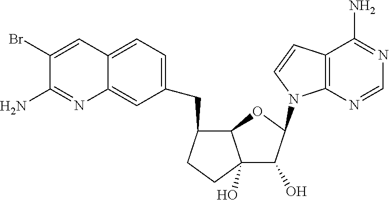 [SMILES: Nc1nc2cc(C[C@@H]3CC[C@@]4(O)[C@@H]3O[C@@H](n3ccc5c(N)ncnc53)[C@@H]4O)ccc2cc1Br]

Step 1: To a stirred solution of (3R,3aS,6aR)-6-((3-bromo-2-((4-methoxybenzyl)amino)quinolin-7-yl)methyl)hexahydro-2H-cyclopenta[b]furan-2,3,3a-triol (70 mg, 0.14 mmol) in dry MeCN (3 mL) was added (E)-diazene-1,2-diylbis(piperidin-1-ylmethanone) (51.4 mg, 0.204 mmol) in MeCN (0.3 mL) dropwise at 0° C. under an atmosphere of argon. This was followed by the addition of tributylphosphine (0.054 mL, 0.22 mmol) dropwise at 0° C. Then the reaction was stirred at 35° C. for 1 h. The resulting solution was used directly in the next step without further purification.

Step 2: To a stirred suspension of 4-chloro-7H-pyrrolo[2,3-d]pyrimidine (39.0 mg, 0.254 mmol) in dry ACN (3 mL) was added DBU (0.039 mL, 0.26 mmol) at room temperature under an argon atmosphere. The resultant solution was stirred at room temperature for 30 minutes. Then the solution from the previous step was transferred via syringe at room temperature under an argon atmosphere. The resulting mixture was stirred at 35° C. for 2 h. The reaction was quenched with brine (20 mL) and extracted with EtOAc (50 mL×3). The combined organic layers were dried over anhydrous sodium sulfate and filtered. The filtrate was concentrated under reduced pressure. The residue was purified by column chromatography on silica (MeOH/DCM) to afford (2R,3R,3aS,6S,6aR)-6-((3-bromo-2-((4-methoxybenzyl)amino)quinolin-7-yl)methyl)-2-(4-chloro-7H-pyrrolo[2,3-d]pyrimidin-7-yl)hexahydro-2H-cyclopenta[b]furan-3,3a-diol. MS: 650/652 (M+1/M+3). ¹H NMR (400 MHz, DMSO-d₆) δ 8.71 (d, J=1.6 Hz, 1H), 8.32 (s, 1H), 8.12 (d, J=4.0 Hz, 1H), 7.55 (d, J=8.3 Hz, 1H), 7.37-7.29 (m, 4H), 7.10-7.05 (m, 1H), 6.86 (d, J=8.2 Hz, 2H), 6.84-6.79 (m, 1H), 6.04 (d, J=8.0 Hz, 1H), 5.37 (brs, 1H), 4.61 (d, J=6.0 Hz, 2H), 4.22 (d, J=8.0 Hz, 1H), 4.08-4.01 (m, 1H), 3.72 (s, 3H), 2.83 (dd, J=13.6, 7.6 Hz, 1H), 2.64 (dd, J=14.6, 7.6 Hz, 1H), 2.37-2.26 (m, 2H), 1.96 (dd, J=12.4, 6.0 Hz, 1H), 1.85-1.75 (m, 1H), 1.71-1.63 (m, 1H), 1.56-1.50 (m, 1H).

Step 3: A solution of (2R,3R,3aS,6S,6aR)-6-((3-bromo-2-((4-methoxybenzyl)amino)quinolin-7-yl)methyl)-2-(4-chloro-7H-pyrrolo[2,3-d]pyrimidin-7-yl)hexahydro-2H-cyclopenta[b]furan-3,3a-diol (60 mg, 0.09 mmol) in TFA (2 mL, 30 mmol) was stirred at 60° C. for 1 h. The mixture was cooled to room temperature and concentrated under reduced pressure. The residue was diluted with EtOAc (100 mL) and washed with saturated aqueous NaHCO₃ (20 mL). The organic layer was dried over anhydrous sodium sulfate and filtered. The filtrate was concentrated under reduced pressure. The residue was purified by colunn chromatography on silica (MeOH/DCM) to afford (2R,3R,3aS,6S,6aR)-6-((2-amino-3-bromoquinolin-7-yl)methyl)-2-(4-chloro-7H-pyrrolo[2,3-d]pyrimidin-7-yl)hexahydro-2H-cyclopenta[b]furan-3,3a-diol. MS: 530/532 (M+1/M+3). ¹H NMR (400 MHz, DMSO-d₆) δ 8.72 (s, 1H), 8.31 (s, 1H), 8.11 (d, J=4.0 Hz, 1H), 7.55 (d, J=8.4 Hz, 1H), 7.29 (s, 1H), 7.10 (dd, J=8.4, 1.6 Hz, 1H), 6.83 (d, J=4.0 Hz, 1H), 6.57-6.51 (m, 3H), 6.03 (d, J=8.0 Hz, 1H), 5.39 (d, J=6.8 Hz, 1H), 5.17 (s, 1H), 4.24 (t, J=7.6 Hz, 1H), 4.05 (d, J=5.6 Hz, 1H), 2.84 (dd, J=13.6, 8.0 Hz, 1H), 2.71-2.60 (m, 1H), 2.37-2.15 (m, 1H), 2.03-1.92 (m, 1H), 1.88-1.63 (m, 1H), 1.62-1.49 (m, 1H).

Step 4: To a sealed tube (20 mL) was added (2R,3R,3aS,6S,6aR)-6-((2-amino-3-bromoquinolin-7-yl)methyl)-2-(4-chloro-7H-pyrrolo[2,3-d]pyrimidin-7-yl)hexahydro-2H-cyclopenta[b]furan-3,3a-diol (30 mg, 0.06 mmol), 1,4-dioxane (5 mL) and NH₃H₂O (9 mL) at room temperature. The vial was sealed, and the reaction was stirred at 90° C. for 16 h. The reaction mixture was cooled to room temperature and concentrated under reduced pressure. The residue was purified by reverse phase HPLC (ACN/water with 5 mM ammonium bicarbonate modifier) to afford (2R,3R,3aS,6S,6aR)-6-((2-amino-3-bromoquinolin-7-yl)methyl)-2-(4-amino-7H-pyrrolo[2,3-d]pyrimidin-7-yl)hexahydro-2H-cyclopenta[b]furan-3,3a-diol. MS: 511/513 (M+1/M+3). ¹H NMR (400 MHz, DMSO-d₆) δ 8.30 (s, 1H), 8.09 (s, 1H), 7.54 (d, J=8.4 Hz, 1H), 7.43 (d, J=4.0 Hz, 1H), 7.28 (s, 1H), 7.09 (d, J=8.4 Hz, 1H), 7.03 (br s, 2H), 6.67 (d, J=3.6 Hz, 1H), 6.54 (br s, 2H), 5.88 (d, J=8.0 Hz, 1H), 5.23 (d, J=7.2 Hz, 1H), 5.04 (s, 1H), 4.13 (t, J=7.6 Hz, 1H), 3.95 (d, J=6.0 Hz, 1H), 2.82 (dd, J=13.2, 8.0 Hz, 1H), 2.62 (dd, J=14.4, 7.2 Hz, 1H), 2.24-2.22 (m, 1H), 1.96-1.92 (m, 1H), 1.70-1.68 (m, 2H), 1.57-1.51 (m, 1H).

### Examples 10 and 11

### (2R,3S,4R,5R)-2-(((2-amino-3-bromoquinolin-7-yl)oxy)methyl)-5-(4-amino-7H-pyrrolo[2,3-d]pyrimidin-7-yl)-3-methyltetrahydrofuran-3,4-diol (Example 10)

### And

### (2R,3S,4R,5R)-2-{[(2-amino-3-bromoquinolin-7-yl)oxy]methyl}-5-(4-chloro-7H-pyrrolo[2,3-d]pyrimidin-7-yl)-3-methyltetrahydrofuran-3,4-diol (Example 11)

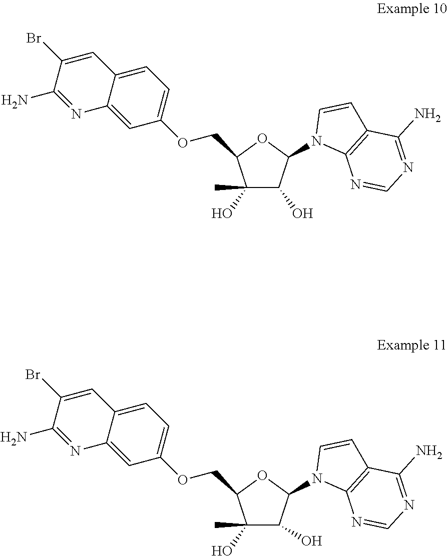 [SMILES: C[C@@]1(O)[C@@H](COc2ccc3cc(Br)c(N)nc3c2)O[C@@H](n2ccc3c(N)ncnc32)[C@@H]1O ; C[C@@]1(O)[C@@H](COc2ccc3cc(Br)c(N)nc3c2)O[C@@H](n2ccc3c(N)ncnc32)[C@@H]1O]

Step 1: Into a 10-L 4-necked round-bottom flask purged and maintained with an inert atmosphere of nitrogen was placed a solution of (5R,6S)-5-(hydroxymethyl)-2,2-dimethyltetrahydrofuro[2,3-d][1,3]dioxol-6-ol (951 g, 5 mol) in pyridine (7 L) and tert-butyl(chloro)diphenylsilane (1.4 kg, 5.1 mol). The resulting solution was stirred overnight at room temperature. The mixture was diluted with MeOH (600 mL) and then concentrated under reduced pressure. The residue was diluted with EtOAc, washed with HCl (0.5 M in water), saturated sodium bicarbonate, and brine. The organic layer was dried over sodium sulfate, filtered, and concentrated under reduced pressure to afford (5R,6S)-5-(((tert-butyldiphenylsilyl)oxy)methyl)-2,2-dimethyltetrahydrofuro[2,3-d][1,3]dioxol-6-ol as an oil, which was used in the next step without further purification.

Step 2: Into a 20-L 4-necked round-bottom flask purged and maintained with an inert atmosphere of nitrogen was placed a solution of (5R,6S)-5-(((tert-butyldiphenylsilyl)oxy)methyl)-2,2-dimethyltetrahydrofuro[2,3-d][1,3]dioxol-6-ol 1(2.1 kg, 5 mol) in DCM (15 L) and Dess-Martin periodinane (3.18 kg, 7.50 mol). The resulting solution was stirred overnight at room temperature. The reaction was quenched by the addition of saturated aqueous sodium bicarbonate, and the solution was concentrated under reduced pressure. The residue was diluted with diethyl ether, and the mixture was filtered. The filtrate was concentrated under reduced pressure to afford (5R)-5-(((tert-butyldiphenylsilyl)oxy)methyl)-2,2-dimethyldihydrofuro[2,3-d][1,3]dioxol-6(5H)-one as an oil which was used in the next step without further purification.

Step 3: Into a 20-L 4-necked round-bottom flask purged and maintained with an inert atmosphere of nitrogen was placed a solution of (5R)-5-(((tert-butyldiphenylsilyl)oxy)methyl)-2,2-dimethyldihydrofuro[2,3-d][1,3]dioxol-6(5H)-one (2.1 kg, 5 mol) in THF (10 L). To this mixture was added methylmagnesium chloride (1.84 L, 3.0 M in THF) dropwise with stirring at 0° C. The resulting solution was stirred for 1 h at room temperature. The reaction was quenched by the addition of saturated aqueous ammonium chloride. The organic layer was washed with brine, dried over anhydrous sodium sulfate, filtered, and concentrated under reduced pressure to afford (5R,6R)-5-(((tert-butyldiphenylsilyl)oxy)methyl)-2,2,6-trimethyltetrahydrofuro[2,3-d][1,3]dioxol-6-ol as an oil, which was used in the next step without further purification. ¹H NMR (500 MHz, CDCl₃) δ 7.73-7.70 (m, 4H), 7.46-7.39 (m, 6H), 5.79 (d, J=3.8 Hz, 1H), 4.16-4.10 (m, 2H), 3.92 (dd, J=6.4, 4.5 Hz, 1H), 3.85-3.84 (m, 2H), 2.56 (s, 1H), 2.07 (s, 1H), 1.38 (s, 3H), 1.28 (t, J=7.1 Hz, 2H), 1.14 (s, 3H), 1.09 (s, 8H).

Step 4: Into a 20-L 4-necked round-bottom flask purged and maintained with an inert atmosphere of nitrogen was placed a solution of (5R,6R)-5-(((tert-butyldiphenylsilyl)oxy)methyl)-2,2,6-trimethyltetrahydrofuro[2,3-d][1,3]dioxol-6-ol (2.2 kg, 5 mol) in AcOH (10 L). To this mixture was added sulfuric acid (49.2 g, 502 mmol) dropwise with stirring at 10° C., followed by acetic anhydride (2.04 kg, 20.0 mol) dropwise with stirring at 10° C. The resulting solution was stirred for 3 h at room temperature. The mixture was concentrated under reduced pressure and then diluted with EtOAc. The resulting mixture was washed with saturated aqueous sodium bicarbonate and then brine. The organic layer was dried over anhydrous sodium sulfate, filtered, and concentrated under reduced pressure. The residue was purified by column chromatography on silica (eluting with 1:1 ethyl acetate/petroleum ether) to afford (3R,4R,5R)-5-(((tert-butyldiphenylsilyl)oxy)methyl)-4-methyltetrahydrofuran-2,3,4-triyl triacetate as an oil.

Step 5: Into a 5-L 4-necked round-bottom flask purged and maintained with an inert atmosphere of nitrogen was placed 4-chloro-5-iodo-7H-pyrrolo[2,3-d]pyrimidine (420 g, 1503 mmol), BSA (305 g, 7.11 mol), a solution of (3R,4R,5R)-5-(((tert-butyldiphenylsilyl)oxy)methyl)-4-methyltetrahydrofuran-2,3,4-triyl triacetate (795 g, 1.50 mol) in MeCN (7 L), and trimethylsilyl trifluoromethanesulfonate (668 g, 3.00 mol). The resulting solution was stirred for 6 h at 80° C. The mixture was cooled to room temperature, concentrated under reduced pressure, and diluted with EtOAc. The resulting mixture was washed with saturated aqueous sodium bicarbonate and brine. The organic layer was dried over anhydrous sodium sulfate, filtered, and concentrated under reduced pressure. The residue was purified by column chromatography on silica (EtOAc/pet. ether) to afford (2R,3R,4R,5R)-2-(((tert-butyldiphenylsilyl)oxy)methyl)-5-(4-chloro-5-iodo-7H-pyrrolo[2,3-d]pyrimidin-7-yl)-3-methyltetrahydrofuran-3,4-diyl diacetate as an oil. MS: 748 (M+1).

Step 6: Into a 5-L 4-necked round-bottom flask purged and maintained with an inert atmosphere of nitrogen was placed a solution of (2R,3R,4R,5R)-2-(((tert-butyldiphenylsilyl)oxy)methyl)-5-(4-chloro-5-iodo-7H-pyrrolo[2,3-d]pyrimidin-7-yl)-3-methyltetrahydrofuran-3,4-diyl diacetate (225 g, 300. mmol) in tetrahydrofuran (2.2 L). The solution was cooled to −78° C., and isopropylmagnesium chloride-lithium chloride complex (54.6 g, 376 mmol) was added dropwise, and the solution was stirred for 2 h at −78° C. The mixture was quenched with dropwise addition of iPrOH (25.2 g, 420 mmol) at −78° C. This cold reaction mixture was poured into a mixture of ice and saturated aqueous ammonium chloride. The mixture was extracted with DCM (2×), and the combined organic layers were dried over sodium sulfate, filtered, and concentrated under reduced pressure to afford (2R,3R,4R,5R)-2-(((tert-butyldiphenylsilyl)oxy)methyl)-5-(4-chloro-7H-pyrrolo[2,3-d]pyrimidin-7-yl)-3-methyltetrahydrofuran-3,4-diyl diacetate as an oil which was used without further purification.

Step 7: Into a 5-L 3-necked round-bottom flask purged and maintained with an inert atmosphere of nitrogen was placed a solution of (2R,3R,4R,5R)-2-(((tert-butyldiphenylsilyl)oxy)methyl)-5-(4-chloro-7H-pyrrolo[2,3-d]pyrimidin-7-yl)-3-methyltetrahydrofuran-3,4-diyl diacetate (186 g, 299 mmol) in THF (1.8 L) and AcOH (90 g, 1.5 mol). TBAF (600 mL, 2.00 equiv, 1.0 M in THF) was added dropwise with stirring at room temperature, and the solution was stirred for 2 h at room temperature. The reaction mixture was concentrated under reduced pressure, and the residue was purified by column chromatography on silica (MeOH/DCM) to afford (2R,3R,4R,5R)-5-(4-chloro-7H-pyrrolo[2,3-d]pyrimidin-7-yl)-2-(hydroxymethyl)-3-methyltetrahydrofuran-3,4-diyl diacetate as a solid. MS: 384 (M+1).

Step 8: A mixture of (2R,3R,4R,5R)-5-(4-chloro-7H-pyrrolo[2,3-d]pyrimidin-7-yl)-2-(hydroxymethyl)-3-methyltetrahydrofuran-3,4-diyl diacetate (542 mg, 1.41 mmol), TEA (0.59 mL, 4.2 mmol), and DMAP (34.5 mg, 0.28 mmol) was dissolved in DCM (11 mL). 4-Toluenesulfonyl chloride (538 mg, 2.8 mmol) was added at 0° C., and the reaction was stirred at room temperature overnight. The mixture was diluted with DCM and washed with water. The aqueous layer was extracted with DCM (2×). The combined organic layers were dried over sodium sulfate, filtered, and concentrated under reduced pressure. The residue was purified by column chromatography on silica (EtOAc/Hexanes) to afford (2R,3R,4R,5R)-5-(4-chloro-7H-pyrrolo[2,3-d]pyrimidin-7-yl)-3-methyl-2-((tosyloxy)methyl)tetrahydrofuran-3,4-diyl diacetate which was used without further purification. MS: 538 (M+1).

Step 9: To a solution of (2R,3R,4R,5R)-5-(4-chloro-7H-pyrrolo[2,3-d]pyrimidin-7-yl)-3-methyl-2-((tosyloxy)methyl)tetrahydrofuran-3,4-diyl diacetate (380 mg, 0.71 mmol) and 2-amino-3-bromoquinolin-7-ol (169 mg, 0.71 mmol) in DMF (5 mL) at 0° C. was added cesium carbonate (460 mg, 1.4 mmol). The mixture was stirred at room temperature overnight and then quenched with water. The precipitate was collected by filtration, rinsed with water, and dried under reduced pressure to afford (2R,3R,4R,5R)-2-{[(2-amino-3-bromoquinolin-7-yl)oxy]methyl}-5-(4-chloro-7H-pyrrolo[2,3-d]pyrimidin-7-yl)-3-methyltetrahydrofuran-3,4-diyl diacetate as a solid. MS: 604/606 (M+1/M+3).

Step 10: To a solution of (2R,3R,4R,5R)-2-{[(2-amino-3-bromoquinolin-7-yl)oxy]methyl}-5-(4-chloro-7H-pyrrolo[2,3-d]pyrimidin-7-yl)-3-methyltetrahydrofuran-3,4-diyl diacetate (372 mg, 0.62 mmol) dissolved in dioxane (4 mL) was added ammonium hydroxide (4 mL, 51.8 mmol, 30% in water). The mixture was heated at 85° C. overnight. The mixture was cooled to room temperature, and the reaction mixture was concentrated under reduced pressure. The residue was purified by mass triggered reverse phase HPLC (ACN/water with 0.1% TFA modifier) to afford: Example 10: (2R,3S,4R,5R)-2-(((2-amino-3-bromoquinolin-7-yl)oxy)methyl)-5-(4-amino-7H-pyrrolo[2,3-d]pyrimidin-7-yl)-3-methyltetrahydrofuran-3,4-diol. MS: 501/503 (M+1/M+3). ¹H NMR (600 MHz, DMSO-d₆) δ 8.72-8.34 (m, 3H), 8.03-7.40 (m, 3H), 7.15-7.03 (m, 2H), 6.94 (d, J=3.0 Hz, 1H), 6.16 (d, J=7.9 Hz, 1H), 5.65-4.96 (m, 2H), 4.40-4.35 (m, 1H), 4.29-4.18 (m, 4H), 1.27 (s, 3H).

Example 11: (2R,3S,4R,5R)-2-{[(2-amino-3-bromoquinolin-7-yl)oxy]methyl}-5-(4-chloro-7H-pyrrolo[2,3-d]pyrimidin-7-yl)-3-methyltetrahydrofuran-3,4-diol. MS: 520/522 (M+1/M+3). ¹H NMR (600 MHz, DMSO-d₆) δ 8.73-8.62 (m, 2H), 8.13 (s, 1H), 7.93 (d, J=3.4 Hz, 1H), 7.76 (d, J=8.7 Hz, 1H), 7.18-7.11 (m, 2H), 6.73 (d, J=3.5 Hz, 1H), 6.25 (d, J=8.0 Hz, 1H), 4.51-4.44 (m, 2H), 4.33-4.20 (m, 5H), 1.28 (s, 3H).

### Example 12

### (2R,3S,4R,5R)-2-(((2-amino-3-bromoquinolin-7-yl)oxy)methyl)-5-(4-methoxy-7H-pyrrolo[2,3-d]pyrimidin-7-yl)-2-methyltetrahydrofuran-3,4-diol

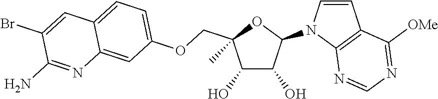 [SMILES: COc1ncnc2c1ccn2[C@@H]1O[C@@](C)(COc2ccc3cc(Br)c(N)nc3c2)[C@@H](O)[C@H]1O]

A solution of 3-bromo-7-(((3aS,4R,6R,6aR)-6-(4-methoxy-7H-pyrrolo[2,3-d]pyrimidin-7-yl)-2,2,4-trimethyltetrahydrofuro[3,4-d][1,3]dioxol-4-yl)methoxy)-N-(4-methoxybenzyl)quinolin-2-amine (30 mg, 0.04 mmol) in TFA (1.0 mL) was stirred at 40° C. for 3 days. The reaction mixture was concentrated in vacuum, and the residue was purified by reverse-phase HPLC (ACN/water with 0.1% TFA modifier) to afford (2R,3S,4R,5R)-2-(((2-amino-3-bromoquinolin-7-yl)oxy)methyl)-5-(4-methoxy-7H-pyrrolo[2,3-d]pyrimidin-7-yl)-2-methyltetrahydrofuran-3,4-diol, TFA salt as a solid. MS: 516/518 (M+1/M+3). ¹H-NMR (600 MHz, DMSO-d₆) δ 8.66 (br, 1H), 8.43 (s, 1H), 7.75 (d, J=8.2 Hz, 1H), 7.63-7.59 (m, 1H), 7.16-7.09 (m, 2H), 6.59-6.54 (m, 1H), 6.23 (d, J=6.6 Hz, 1H), 4.88-4.81 (m, 1H), 4.27-4.19 (m, 2H), 4.11 (d, J=9.9 Hz, 1H), 4.03 (s, 3H), 1.36 (s, 3H).

### Example 13

### (2R,3S,4R,5R)-2-(((2-amino-3-bromoquinolin-7-yl)oxy)methyl)-5-(4-amino-7H-pyrrolo[2,3-d]pyrimidin-7-yl)-2-methyltetrahydrofuran-3,4-diol

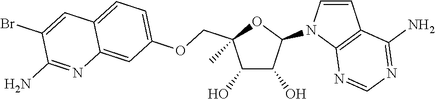 [SMILES: C[C@@]1(COc2ccc3cc(Br)c(N)nc3c2)O[C@@H](n2ccc3c(N)ncnc32)[C@H](O)[C@@H]1O]

A solution of (2R,3S,4R,5R)-2-(((2-amino-3-bromoquinolin-7-yl)oxy)methyl)-5-(4-methoxy-7H-pyrrolo[2,3-d]pyrimidin-7-yl)-2-methyltetrahydrofuran-3,4-diol, TFA salt (10 mg, 0.019 mmol) in 30% ammonia in water (3 mL) was stirred at 150° C. for 4 h in a microwave reactor. The reaction mixture was concentrated under reduced pressure, and the residue was purified by chiral SFC (DIOL column, 35%/65% methanol/CO₂) to afford (2R,3S,4R,5R)-2-(((2-amino-3-bromoquinolin-7-yl)oxy)methyl)-5-(4-amino-7H-pyrrolo[2,3-d]pyrimidin-7-yl)-2-methyltetrahydrofuran-3,4-diol as a solid. MS: 501/503 (M+1/M+3). ¹H-NMR (500 MHz, CD₃OD) δ 8.22 (s, 1H), 8.09 (s, 1H), 7.58 (d, J=9.0 Hz, 1H), 7.35 (d, J=3.7 Hz, 1H), 7.06-6.98 (m, 2H), 6.60 (d, J=3.3 Hz, 1H), 6.27 (d, J=6.7 Hz, 1H), 4.87-4.82 (m, 1H), 4.39 (d, J=5.4 Hz, 1H), 4.20 (d, J=10.2 Hz, 1H), 4.12 (d, J=10.2 Hz, 1H), 1.48 (s, 3H).

### Example 14

### (2R,3S,4R,5R)-2-(((2-amino-3-bromoquinolin-7-yl)oxy)methyl)-5-(4-amino-7H-pyrrolo[2,3-d]pyrimidin-7-yl)-3-methyltetrahydrofuran-3,4-diol

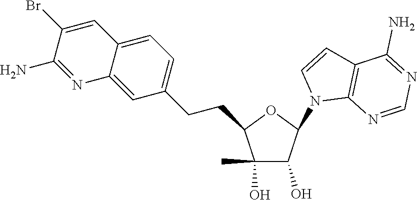 [SMILES: C[C@@]1(O)[C@@H](CCc2ccc3cc(Br)c(N)nc3c2)O[C@@H](n2ccc3c(N)ncnc32)[C@@H]1O]

Step 1: A 500 mL round bottom flask was charged with (3aR,5R,6aR)-5-((R)-2,2-dimethyl-1,3-dioxolan-4-yl)-2,2-dimethyltetrahydrofuro[2,3-d][1,3]dioxol-6-ol (10.0 g, 38.4 mmol), which was then dissolved in DCM (100 mL). Then Dess-Martin Periodinane (32.6 g, 77 mmol) was added portion-wise, and the cloudy reaction was stirred at room temperature for 80 min. DCM (100 mL) was added, and the reaction was stirred at room temperature overnight. The reaction was quenched with saturated aqueous sodium bicarbonate and saturated aqueous sodium thiosulfate. After 1 h with stirring, the layers were extracted, and the organic layer was dried over magnesium sulfate, filtered, and concentrated under reduced pressure to afford crude (3aR,5S,6aS)-5-((R)-2,2-dimethyl-1,3-dioxolan-4-yl)-2,2-dimethyldihydrofuro[2,3-d][1,3]dioxol-6(3aH)-one as an oil, which was used directly in the next step without further purification. ¹H-NMR (600 MHz, CDCl₃) δ 6.14 (d, J=4.5 Hz, 1H), 4.39-4.38 (m, 1H), 4.38-4.34 (m, 2H), 4.04-4.01 (m, 2H), 1.46 (s, 3H), 1.43 (s, 3H), 1.35-1.32 (m, 6H).

Step 2: A flask was charged with crude (3aR,5R,6aS)-5-((S)-2,2-dimethyl-1,3-dioxolan-4-yl)-2,2-dimethyldihydrofuro[2,3-d][1,3]dioxol-6(3aH)-one (7.42 g, max 28.7 mmol), backfilled with argon, and then toluene (100 mL) was added. The solution was cooled to 0° C., and then methyl magnesium chloride (3.0 M in THF, 14.4 mL, 43.2 mmol) was added dropwise under an argon atmosphere. After 5 minutes, the reaction was removed from the ice bath and allowed to warm to room temperature, stirring overnight. The reaction was poured into a separatory funnel containing saturated ammonium chloride and extracted with EtOAc. The organic layers were combined and washed with brine, dried over magnesium sulfate, filtered, and concentrated under reduced pressure to afford (3aR,5R,6R,6aR)-5-((S)-2,2-dimethyl-1,3-dioxolan-4-yl)-2,2,6-trimethyltetrahydrofuro[2,3-d][1,3]dioxol-6-ol as a solid, which was used in the next step without further purification. ¹H-NMR (600 MHz, CDCl₃) δ 5.70 (d, J=3.6 Hz, 1H), 4.17 (d, J=3.7 Hz, 1H), 4.12-4.08 (m, 2H), 3.95-3.91 (m, 1H), 3.78 (d, J=7.4 Hz, 1H), 2.67 (s, 1H), 1.59 (s, 3H), 1.45 (s, 3H), 1.36 (s, 3H), 1.35 (s, 3H), 1.28 (s, 3H).

Step 3: To flask charged with (3aR,5R,6R,6aR)-5-((S)-2,2-dimethyl-1,3-dioxolan-4-yl)-2,2,6-trimethyltetrahydrofuro[2,3-d][1,3]dioxol-6-ol (5.82 g, 21.2 mmol) was added acetonitrile (100 mL). Then sulfuric acid (10 mL, 188 mmol) as a 5 vol % in water solution was added, and the reaction was stirred at room temperature for 4.5 h. The reaction was poured into a separatory funnel containing saturated sodium bicarbonate and extracted with EtOAc. The organic layers were combined and washed with brine, dried over sodium sulfate, filtered, and concentrated under reduced pressure to afford (S)-1-((3aR,5R,6R,6aR)-6-hydroxy-2,2,6-trimethyltetrahydrofuro[2,3-d][1,3]dioxol-5-yl)ethane-1,2-diol as a solid, which was used in the next reaction without further purification. ¹H-NMR (600 MHz, CDCl₃) δ 5.73 (d, J=3.7 Hz, 1H), 4.16 (d, J=3.7 Hz, 1H), 3.85-3.81 (m, 2H), 3.75 (d, J=8.4 Hz, 1H), 3.72-3.68 (m, 1H), 2.87 (s, 1H), 2.53 (s, 1H), 2.02 (s, 1H), 1.59 (s, 3H), 1.36 (s, 3H), 1.33 (s, 3H).

Step 4: To a flask containing a solution of crude (S)-1-((3aR,5R,6R,6aR)-6-hydroxy-2,2,6-trimethyltetrahydrofuro[2,3-d][1,3]dioxol-5-yl)ethane-1,2-diol (1.24 g, 5.30 mmol) in toluene (100 mL) was sequentially added imidazole (1.44 g, 21.2 mmol) and triphenylphosphine (5.56 g, 21.2 mmol). The flask was cooled to 0° C., and then iodine (4.04 g, 15.9 mmol) was added. The ice bath was allowed to naturally expire, and the solution was then stirred at room temperature under an atmosphere of argon for four days. The reaction was poured into a separatory funnel containing 1M NaOH and extracted with EtOAc. The organic layers were combined and washed with brine, dried over magnesium sulfate, filtered, and concentrated under reduced pressure. The residue was purified by column chromatography on silica (0-10-20-30% EtOAc/hexanes) to afford (3aR,5R,6R,6aR)-2,2,6-trimethyl-5-vinyltetrahydrofuro[2,3-d][1,3]dioxol-6-ol as a solid. ¹H-NMR (600 MHz, CDCl₃) δ 5.84-5.76 (m, 2H), 5.40 (dt, J=17.3, 1.5 Hz, 1H), 5.29 (dt, J=10.7, 1.4 Hz, 1H), 4.21 (d, J=5.9 Hz, 1H), 4.17 (d, J=3.9 Hz, 1H), 2.63 (s, 1H), 1.59 (s, 3H), 1.37 (s, 3H), 1.12 (s, 3H).

Step 5: A vial charged with (3aR,5R,6R,6aR)-2,2,6-trimethyl-5-vinyltetrahydrofuro[2,3-d][1,3]dioxol-6-ol (200 mg, 0.999 mmol) and 9-BBN (0.5 M in THF, 6 mL, 3.00 mmol) was stirred at 50° C. for 2 h under an atmosphere of argon. The reaction was cooled to room temperature, and tripotassium phosphate (2M in water, 2.5 mL, 5.00 mmol) was added. The mixture was stirred vigorously at room temperature for 30 minutes, and then a solution of 3-bromo-7-iodo-N-(4-methoxybenzyl)quinolin-2-amine (609 mg, 1.298 mmol) and 1,1′-bis(diphenylphosphino)ferrocene-palladium(II)dichloride dichloromethane complex (82 mg, 0.100 mmol) in THF (3 mL) was added. The vial headspace was purged with argon, and the reaction was stirred at 50° C. overnight. The reaction was diluted with DCM and water and passed through a phase separator. The organic layers were combined and concentrated under reduced pressure. The residue was purified by column chromatography on silica (0-60% EtOAc/hexanes) to afford (3aR,5R,6R,6aR)-5-(2-(3-bromo-2-((4-methoxybenzyl)amino)quinolin-7-yl)ethyl)-2,2,6-trimethyltetrahydrofuro[2,3-d][1,3]dioxol-6-ol as a solid. MS: 543/545 (M+1/M+3).

Step 6: To a vial containing (3aR,5R,6R,6aR)-5-(2-(3-bromo-2-((4-methoxybenzyl)amino)quinolin-7-yl)ethyl)-2,2,6-trimethyltetrahydrofuro[2,3-d][1,3]dioxol-6-ol (353.2 mg, 0.650 mmol) was added water (5 mL), followed by neat formic acid (5 mL, 130 mmol). The resulting solution was heated at 50° C. overnight. The reaction was cooled to room temperature, and then poured into a separatory funnel containing water and extracted with 25% isopropanol/chloroform. The organic layers were combined and dried over sodium sulfate, filtered, and concentrated under reduced pressure to afford (2S,3R,4S,5R)-5-(2-(3-bromo-2-((4-methoxybenzyl)amino)quinolin-7-yl)ethyl)-4-methyltetrahydrofuran-2,3,4-triol as a solid, which was used in the next step without further purification. MS: 503/505 (M+1/M+3).

Step 7: In a vial, 4-chloro-7H-pyrrolo[2,3-d]pyrimidine (405 mg, 2.64 mmol) was dissolved in DMF (8 mL). Then sodium hydride (106 mg, 2.64 mmol) was added. The mixture was stirred at room temperature under an atmosphere of argon for 75 minutes. Concurrently, to another vial was added a solution of crude (3R,4S,5R)-5-(2-(3-bromo-2-((4-methoxybenzyl)amino)quinolin-7-yl)ethyl)-4-methyltetrahydrofuran-2,3,4-triol (532 mg, 1.06 mmol) in acetonitrile (18 mL). Then tri-n-butylphosphine (0.45 mL, 1.80 mmol) was added, followed by 1,1′-(azodicarbonyl)dipiperidine (400 mg, 1.58 mmol). The reaction was stirred at room temperature for 25 minutes before additional 1,1′-(azodicarbonyl)dipiperidine (130 mg) was added. 20 minutes later, more tri-n-butylphosphine (0.5 mL) was added. This mixture was stirred at room temperature for another 15 min. Then the solution containing 4-chloro-7H-pyrrolo[2,3-d]pyrimidine and sodium hydride was taken up by syringe and added to the solution initially containing (3R,4S,5R)-5-(2-(3-bromo-2-((4-methoxybenzyl)amino)quinolin-7-yl)ethyl)-4-methyltetrahydrofuran-2,3,4-triol. This combined reaction was stirred at room temperature under an atmosphere of argon overnight. The reaction was poured into a separatory funnel containing water and extracted with EtOAc. The organic layers were combined, washed with water twice and then brine, dried over magnesium sulfate, filtered, and concentrated under reduced pressure. The residue was purified by column chromatography on silica (20-90% EtOAc/hexanes) to afford (2R,3S,4R,5R)-2-(2-(3-bromo-2-((4-methoxybenzyl)amino)quinolin-7-yl)ethyl)-5-(4-chloro-7H-pyrrolo[2,3-d]pyrimidin-7-yl)-3-methyltetrahydrofuran-3,4-diol as a solid, which was used directly in the next step. MS: 638/640 (M+1/M+3).

Step 8: To a vial was added a solution of (2R,3S,4R,5R)-2-(2-(3-bromo-2-((4-methoxybenzyl)amino)quinolin-7-yl)ethyl)-5-(4-chloro-7H-pyrrolo[2,3-d]pyrimidin-7-yl)-3-methyltetrahydrofuran-3,4-diol (114 mg, 0.178 mmol) in ammonia (7 M in MeOH, 10 mL, 70.0 mmol) and dioxane (2 mL). This solution was heated at 130° C. for 4 h in a microwave reactor. The entire solution was then concentrated under reduced pressure to afford (2R,3S,4R,5R)-5-(4-amino-7H-pyrrolo[2,3-d]pyrimidin-7-yl)-2-(2-(3-bromo-2-((4-methoxybenzyl)amino)quinolin-7-yl)ethyl)-3-methyltetrahydrofuran-3,4-diol, which was used in the next step without further purification. MS: 619/621 (M+1/M+3).

Step 9: To a flask containing crude (2R,3S,4R,5R)-5-(4-amino-7H-pyrrolo[2,3-d]pyrimidin-7-yl)-2-(2-(3-bromo-2-((4-methoxybenzyl)amino)quinolin-7-yl)ethyl)-3-methyltetrahydrofuran-3,4-diol (110 mg, 0.178 mmol) was added DCM (9 mL). Then trifluoroacetic acid (1 mL, 12.98 mmol) was added, and the reaction was stirred at room temperature overnight. TFA (5 mL) was added, and the reaction was stirred at room temperature for another 1 h. TFA (2 mL) was added, and the reaction was refluxed for 7 h. The reaction was cooled to room temperature, concentrated under reduced pressure. The residue was purified by mass-triggered reverse phase HPLC (ACN/water with 0.1% TFA modifier) to afford (2R,3S,4R,5R)-2-(2-(2-amino-3-bromoquinolin-7-yl)ethyl)-5-(4-amino-7H-pyrrolo[2,3-d]pyrimidin-7-yl)-3-methyltetrahydrofuran-3,4-diol as a solid TFA salt. MS: 499/501 (M+1/M+3). ¹H-NMR (600 MHz, DMSO-d₆) δ 8.64 (s, 1H), 8.39 (s, 1H), 7.74 (d, J=3.7 Hz, 1H), 7.71 (d, J=8.2 Hz, 1H), 7.39 (s, 1H), 7.25 (d, J=8.0 Hz, 1H), 7.00 (d, J=3.7 Hz, 1H), 6.10 (d, J=7.7 Hz, 1H), 4.31 (d, J=7.7 Hz, 1H), 3.81 (dd, J=11.4, 2.9 Hz, 1H), 2.81 (ddd, J=13.8, 9.2, 4.7 Hz, 1H), 2.67 (dt, J=13.8, 8.1 Hz, 1H), 2.07-1.99 (m, 1H), 1.88-1.81 (m, 1H), 1.20 (s, 3H).

### Example 15

### (1S,2R,3S,5R)-3-[2-(2-amino-3-bromo-7-quinolinyl)ethyl]-5-(4-amino-7H-pyrrolo[2,3-d]pyrimidin-7-yl)-3-methyl-1,2-cyclopentanediol

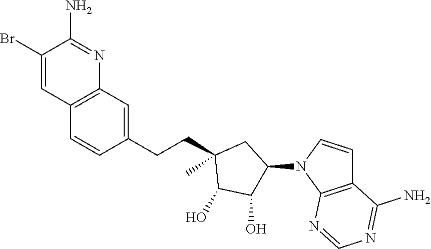 [SMILES: C[C@]1(CCc2ccc3cc(Br)c(N)nc3c2)C[C@@H](n2ccc3c(N)ncnc32)[C@H](O)[C@@H]1O]

Step 1: To a stirred solution of (3a′R,4′R,6'S,6a'S)-4′-methyl-4′-vinyltetrahydro-4′H-spiro[cyclohexane-1,2′-cyclopenta[d][1,3]dioxol]-6′-ol (3.8 g, 16 mmol) in DCM (80 mL) was added pyridine (6.45 mL, 80 mmol). The mixture was cooled to 0° C. and treated with trifluoromethanesulfonic anhydride in DCM (23.92 mL, 23.92 mmol) over 10 min. The mixture was stirred at 0° C. for 30 minutes and treated with water (5 mL). The organic layers were separated and washed with brine. The organic layers were combined, dried over sodium sulfate and concentrated under reduced pressure. Toluene (10 mL) was added to the residue and concentrated to afford (3a′R,4'S,6′R,6a′R)-6′-methyl-6′-vinyltetrahydro-3a′H-spiro[cyclohexane-1,2′-cyclopenta[d][1,3]dioxol]-4′-yl trifluoromethanesulfonate as an oil. The residue was used in the next step without further purification.

Step 2: To a stirred solution of 4-chloro-7H-pyrrolo[2,3-d]pyrimidine (2.94 g, 19.1 mmol) in DMF (35 mL) at 0° C. was added sodium hydride (0.893 g, 22.3 mmol). The mixture was stirred at that temperature for 30 minutes. In a separate flask, (3a′R,4'S,6′R,6a′R)-6′-methyl-6′-vinyltetrahydro-3a′H-spiro[cyclohexane-1,2′-cyclopenta[d][1,3]dioxol]-4′-yl trifluoromethanesulfonate (5.90 g, 15.9 mmol) was dissolved in DMF (10 mL). The solution was added to the solution of the sodium salt slowly over 10 minutes. The resultant mixture was warmed to room temperature and stirred overnight. The mixture was cooled to 0° C. and treated with water. The mixture was diluted with EtOAc (500 mL) and washed with water (3×) and brine. The organic layer was dried over sodium sulfate, concentrated, and purified by column chromatography on silica (0-20% EtOAc/DCM) to afford 4-chloro-7-((3a′R,4′R,6′R,6a'S)-4′-methyl-4′-vinyltetrahydro-4′H-spiro[cyclohexane-1,2′-cyclopenta[d][1,3]dioxol]-6′-yl)-7H-pyrrolo[2,3-d]pyrimidine. MS: 374 (M+1). ¹H-NMR (500 MHz, DMSO-d₆) δ 8.66 (s, 1H), 8.03 (d, J=3.2 Hz, 1H), 6.73 (d, J=3.2 Hz, 1H), 6.00 (dd, J=17.4, 10.7 Hz, 1H), 5.32-5.26 (m, 1H), 5.10-4.99 (m, 3H), 4.63 (d, J=7.5 Hz, 1H), 2.46 (m, 1H), 2.08 (dd, J=12.7, 7.2 Hz, 1H), 1.75-1.71 (m, 2H), 1.63-1.28 (m, 8H), 1.17 (s, 3H).

Step 3: To a stirred solution of 4-chloro-7-((3a′R,4′R,6′R,6a'S)-4′-methyl-4′-vinyltetrahydro-4′H-spiro[cyclohexane-1,2′-cyclopenta[d][1,3]dioxol]-6′-yl)-7H-pyrrolo[2,3-d]pyrimidine (910 mg, 2.43 mmol) in 1,4-dioxane (4 mL) was added ammonium hydroxide (28%, 4 mL) in a microwave vial. The reaction was heated to 160° C. for 5 h in a microwave reactor. The mixture was cooled to room temperature, and diluted with EtOAc and water. The aqueous layer was extracted with EtOAc. The organic layers were combined and washed with brine, dried over sodium sulfate, and concentrated under reduced pressure to afford 7-((3a′R,4′R,6′R,6a'S)-4′-methyl-4′-vinyltetrahydro-4′H-spiro[cyclohexane-1,2′-cyclopenta[d][1,3]dioxol]-6′-yl)-7H-pyrrolo[2,3-d]pyrimidin-4-amine. The crude product was used in the next step without further purification. MS: 355 (M+1). ¹H NMR (500 MHz, DMSO-d₆) δ 8.06 (s, 1H), 7.37 (d, J=2.8 Hz, 1H), 6.99 (s, 2H), 6.57 (d, J=2.8 Hz, 1H), 5.98 (dd, J=17.4, 10.7 Hz, 1H), 5.18-4.93 (m, 4H), 4.59 (d, J=7.6 Hz, 1H), 2.37 (dd, J=12.3 Hz, 1H), 2.00-1.95 (m, 1H), 1.74-1.28 (m, 10H), 1.14 (s, 3H).

Step 4: To 7-((3a′R,4′R,6′R,6a'S)-4′-methyl-4′-vinyltetrahydro-4′H-spiro[cyclohexane-1,2′-cyclopenta[d][1,3]dioxol]-6′-yl)-7H-pyrrolo[2,3-d]pyrimidin-4-amine (300 mg, 0.846 mmol) was added 9-BBN (0.5 M in THF, 6.77 mL, 3.39 mmol). The mixture was heated to 50° C. for 1 h, cooled to room temperature, treated with potassium phosphate tribasic (898 mg, 4.23 mmol) and water (0.9 mL), and left to stir for 30 minutes. The mixture was treated with THF (1 mL), 3-bromo-7-iodoquinolin-2-amine (266 mg, 0.762 mmol), and PdCl₂(dppf) (61.9 mg, 0.085 mmol), purged with nitrogen for 5 minutes, and heated to 50° C. for 3 h. The mixture was cooled to room temperature, diluted with EtOAc, and washed with water and brine. The organic layers were combined and dried over sodium sulfate, concentrated, and purified by column chromatography on silica (0-10% MeOH/DCM) to afford 7-(2-((3a'S,4′R,6'S,6a′R)-4′-(4-amino-7H-pyrrolo[2,3-d]pyrimidin-7-yl)-6′-methyltetrahydro-4′H-spiro[cyclohexane-1,2′-cyclopenta[d][1,3]dioxol]-6′-yl)ethyl)-3-bromoquinolin-2-amine as a solid. MS: 577/579 (M+1/M+3). ¹H-NMR (500 MHz, DMSO-d₆) δ 8.32 (s, 1H), 8.06 (s, 1H), 7.57 (d, J=8.1 Hz, 1H), 7.38 (s, 1H), 7.32 (s, 1H), 7.12 (d, J=8.1 Hz, 1H), 6.99 (s, 2H), 6.57 (s, 3H), 5.14-5.07 (m, 1H), 4.98-4.94 (m, 1H), 4.52 (d, J=7.5 Hz, 1H), 2.75-2.65 (m, 2H), 2.20 (dd J=12.4 Hz, 1H), 2.05-2.00 (m, 1H), 1.78-1.28 (m, 12H), 1.14 (s, 3H).

Step 5: To 7-(2-((3a'S,4′R,6'S,6a′R)-4′-(4-amino-7H-pyrrolo[2,3-d]pyrimidin-7-yl)-6′-methyltetrahydro-4′H-spiro[cyclohexane-1,2′-cyclopenta[d][1,3]dioxol]-6′-yl)ethyl)-3-bromoquinolin-2-amine (320 mg, 0.399 mmol) was added HCl (4M in MeOH, 10 mL). The mixture was stirred overnight, and then heated at 50° C. for 5 h. The reaction mixture was cooled to room temperature, concentrated under reduced pressure, and purified by reverse phase column chromatography (ACN/water with 0.1% TFA modifier) to afford (1S,2R,3S,5R)-3-[2-(2-amino-3-bromo-7-quinolinyl)ethyl]-5-(4-amino-7H-pyrrolo[2,3-d]pyrimidin-7-yl)-3-methyl-1,2-cyclopentanediol as the TFA salt. MS: 497/499 (M+1/M+3). ¹H NMR (500 MHz, DMSO-d₆) δ 9.17 (brs, 1H), 8.72 (s, 1H), 8.65 (br s, 1H), 8.37 (s, 1H), 8.27 (brs, 2H), 7.76 (d, J=8.1 Hz, 1H), 7.70 (d, J=3.2 Hz, 1H), 7.46 (s, 1H), 7.34 (d, J=8.1 Hz, 1H), 6.94 (d, J=3.1 Hz, 1H), 5.00 (dd J=8.8 Hz, 1H), 4.89 (brs, 2H), 4.39-4.34 (m, 1H), 3.79 (d, J=5.9 Hz, 1H), 2.85-2.68 (m, 2H), 1.96-1.70 (m, 4H), 1.12 (s, 3H).

### Example 16

### (1S,2R,3aR,4S,6aR)-4-((2-amino-3-fluoroquinolin-7-yl)methyl)-2-(4-methyl-7H-pyrrolo[2,3-d]pyrimidin-7-yl)hexahydropentalene-1,6a(1H)-diol

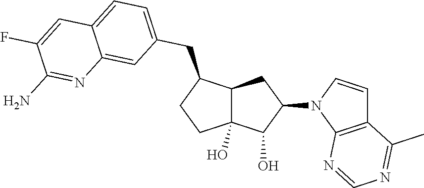 [SMILES: Cc1ncnc2c1ccn2[C@@H]1C[C@@H]2[C@H](Cc3ccc4cc(F)c(N)nc4c3)CC[C@]2(O)[C@H]1O]

Step 1: To a flask containing (3aS,4R,5aR,6S,8aR)-2,2-dimethyl-4-(4-methyl-7H-pyrrolo[2,3-d]pyrimidin-7-yl)hexahydro-5H-pentaleno[1,6a-d][1,3]dioxol-6-ol (6.0 g, 18 mmol) was added DCM (200 mL), followed by Dess-Martin Periodinane (10.25 g, 23.68 mmol). The reaction was stirred at room temperature overnight. The reaction was quenched with 1:1 saturated aqueous sodium bicarbonate:saturated aqueous sodium thiosulfate (160 mL) with vigorous stirring. The organic layer was separated by Phase Separator and concentrated under reduced pressure. The crude (3aS,4R,5aS,8aR)-2,2-dimethyl-4-(4-methyl-7H-pyrrolo[2,3-d]pyrimidin-7-yl)hexahydro-6H-pentaleno[1,6a-d][1,3]dioxol-6-one was used directly in the next reaction.

Step 2: To a flask was added methyltriphenylphosphonium bromide (21.91 g, 60.1 mmol), followed by THF (100 mL) under an atmosphere of argon. The mixture was cooled to 0° C., and then nBuLi (22 mL, 2.5M, 55 mmol) was added. The reaction was brought out of the cold bath and allowed to vigorously stir at room temperature under argon for 30 minutes. Then the reaction was cooled back down to 0° C., and a solution of (3aS,4R,5aS,8aR)-2,2-dimethyl-4-(4-methyl-7H-pyrrolo[2,3-d]pyrimidin-7-yl)hexahydro-6H-pentaleno[1,6a-d][1,3]dioxol-6-one (5.96 g, 18.2 mmol) in THF (100 mL) was added. After addition, the reaction was brought out of the cold bath and allowed to stir at room temperature for 70 min. The reaction was poured into a separatory funnel containing EtOAc and saturated ammonium chloride. The aqueous layer was separated and washed twice with EtOAc. The organic layers were combined, dried over magnesium sulfate, filtered over Celite®, and concentrated under reduced pressure. The crude material was subjected to column chromatography on silica (10-100% EtOAc/hexanes) to afford 7-((3aS,4R,5aR,8aR)-2,2-dimethyl-6-methylenehexahydro-5H-pentaleno[1,6a-d][1,3]dioxol-4-yl)-4-methyl-7H-pyrrolo[2,3-d]pyrimidine. MS: 326 (M+1). ¹H NMR (600 MHz, CDCl₃) δ 8.76 (s, 1H), 7.24 (d, J=3.4 Hz, 1H), 6.55 (d, J=3.4 Hz, 1H), 5.16-5.10 (m, 1H), 5.00-4.97 (m, 1H), 4.87 (s, 1H), 4.80 (d, J=5.3 Hz, 1H), 3.08 (t, J=9.1 Hz, 1H), 2.73 (s, 3H), 2.71-2.61 (m, 2H), 2.58-2.51 (m, 1H), 2.32-2.24 (m, 2H), 2.15-2.08 (m, 1H), 1.58 (s, 3H), 1.36 (s, 3H).

Step 3: To a flask containing 7-((3aS,4R,5aR,8aR)-2,2-dimethyl-6-methylenehexahydro-5H-pentaleno[1,6a-d][1,3]dioxol-4-yl)-4-methyl-7H-pyrrolo[2,3-d]pyrimidine (1.22 g, 3.7 mmol) was added THF (20 mL). Then 9-BBN (23.0 mL, 0.5M in THF, 11.5 mmol) solution was added under an atmosphere of argon. The reaction was stirred at room temperature under argon for overnight. Then tripotassium phosphate (9.5 mL, 2M in water, 19 mmol) was added, and the reaction was stirred at room temperature for 30 minutes. Then a solution of 7-bromo-3-fluoroquinolin-2-amine (1.345 g, 5.60 mmol) and methanesulfonato(2-dicyclohexylphosphino-2′,6′-diisopropoxy-1,1′-biphenyl)(2′-methylamino-1,1′-biphenyl-2-yl)palladium(II) (0.312 g, 0.373 mmol) in THF (10 mL) was added, and the reaction was heated to 50° C. for 2 h. The reaction was cooled to room temperature, and then concentrated under reduced pressure. The crude material was purified by column chromatography on silica (80-100% EtOAc/hexanes to 100% 3:1 EtOAc:EtOH) followed by chiral SFC (OJ-H column, 30% MeOH w/0.1% NH₄OH in CO₂) to afford 7-(((3aS,4R,5aR,6S,8aR)-2,2-dimethyl-4-(4-methyl-7H-pyrrolo[2,3-d]pyrimidin-7-yl)hexahydro-5H-pentaleno[1,6a-d][1,3]dioxol-6-yl)methyl)-3-fluoroquinolin-2-amine. MS: 488 (M+1).

Step 4: To a flask containing 7-(((3aS,4R,5aR,6S,8aR)-2,2-dimethyl-4-(4-methyl-7H-pyrrolo[2,3-d]pyrimidin-7-yl)hexahydro-5H-pentaleno[1,6a-d][1,3]dioxol-6-yl)methyl)-3-fluoroquinolin-2-amine (2.03 g, 4.17 mmol) were added DCM (50 mL), water (16 mL), and trifluoroacetic acid (40 mL, 520 mmol). The reaction was stirred at room temperature overnight. The reaction was concentrated under reduced pressure and purified by mass-triggered reverse phase HPLC (ACN/water with 0.1% NH₄OH modifier) to afford (1S,2R,3aR,4S,6aR)-4-((2-amino-3-fluoroquinolin-7-yl)methyl)-2-(4-methyl-7H-pyrrolo[2,3-d]pyrimidin-7-yl)hexahydropentalene-1,6a(1H)-diol. MS: 448 (M+1). ¹H NMR (600 MHz, DMSO) δ 8.60 (s, 1H), 7.82 (d, J=3.6 Hz, 1H), 7.75 (d, J=11.8 Hz, 1H), 7.55 (d, J=8.2 Hz, 1H), 7.29 (s, 1H), 7.10 (d, J=8.6 Hz, 1H), 6.71 (d, J=3.6 Hz, 1H), 6.67 (s, 2H), 4.84-4.74 (m, 2H), 4.67 (s, 1H), 3.95 (dd, J=10.2, 7.4 Hz, 1H), 2.76-2.66 (m, 2H), 2.65 (s, 3H), 2.49-2.42 (m, 1H), 2.18 (q, J=9.4 Hz, 1H), 1.88-1.78 (m, 3H), 1.71-1.64 (m, 1H), 1.64-1.55 (m, 1H), 1.49-1.43 (m, 1H).

Examples 17-18: Examples 17-18 in Table 7 were synthesized in an analogously as described in example 16 by substituting 7-bromo-3-fluoroquinolin-2-amine with an appropriate aryl-halide in step 3.

TABLE 7

| Ex | Structure | Name | MS |
|---|---|---|---|
| 17 | 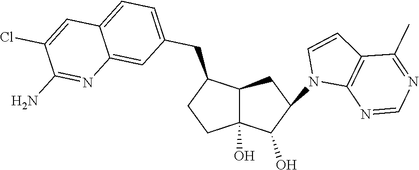 [SMILES: Cc1ncnc2c1ccn2[C@@H]1C[C@@H]2[C@H](Cc3ccc4cc(Cl)c(N)nc4c3)CC[C@]2(O)[C@H]1O] | (1S,2R,3aR,4S,6aR)-4-((2- amino-3-chloroquinolin-7- yl)methyl)-2-(4-methyl-7H- pyrrolo[2,3-d]pyrimidin-7- yl)hexahydropentalene- 1,6a(1H)-diol | 464 (M + 1) |
| 18 | 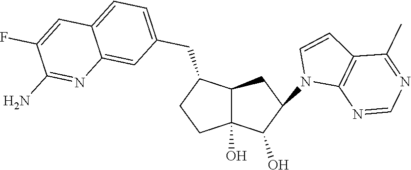 [SMILES: Cc1ncnc2c1ccn2[C@@H]1C[C@@H]2[C@@H](Cc3ccc4cc(F)c(N)nc4c3)CC[C@]2(O)[C@H]1O] | (1S,2R,3aR,4R,6aR)-4-((2- amino-3-fluoroquinolin-7- yl)methyl)-2-(4-methyl-7H- pyrrolo[2,3-d]pyrimidin-7- yl)hexahydropentalene- 1,6a(1H)-diol | 448 (M + 1) |

Examples 19: Example 19 in Table 8 was synthesized in an analogously to Example 16 by substituting step 3 with step 1 in Example 25, and substituting 7-bromo-3,5-difluoroquinolin-2-amine with an appropriate aryl-halide.

TABLE 8

| Ex | Structure | Name | MS |
|---|---|---|---|
| 19 | 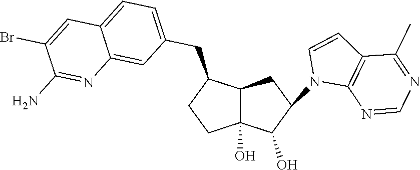 [SMILES: Cc1ncnc2c1ccn2[C@@H]1C[C@@H]2[C@H](Cc3ccc4cc(Br)c(N)nc4c3)CC[C@]2(O)[C@H]1O] | (1S,2R,3aR,4S,6aR)-4-((2- amino-3-bromoquinolin-7- yl)methyl)-2-(4-methyl-7H- pyrrolo[2,3-d]pyrimidin-7- yl)hexahydropentalene- 1,6a(1H)-diol | 508/510 (M + 1/M + 3) |

Examples 20-24: Examples 20-24 in Table 9 were synthesized in an analogously to steps 3-4 of Example 16 by substituting the 7-((3aS,4R,5aR,8aR)-2,2-dimethyl-6-methylenehexahydro-5H-pentaleno[1,6a-d][1,3]dioxol-4-yl)-4-methyl-7H-pyrrolo[2,3-d]pyrimidine with an appropriate exo-olefin and 7-bromo-3-fluoroquinolin-2-amine with an appropriate aryl-halide and followed by chiral resolution by SFC if needed based on the substituted exo-olefin.

TABLE 9

| Ex | Structure | Name | MS |
|---|---|---|---|
| 20 | 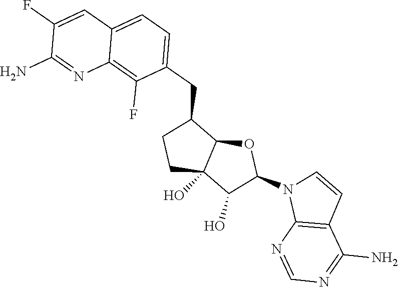 [SMILES: Nc1nc2c(F)c(C[C@@H]3CC[C@@]4(O)[C@@H]3O[C@@H](n3ccc5c(N)ncnc53)[C@@H]4O)ccc2cc1F] | (2R,3R,3aS,6S,6aR)-6-[(2-amino- 3,8-difluoroquinolin-7- yl)methyl]-2-(4-amino-7H- pyrrolo[2,3-d]pyrimidin-7- yl)hexahydro-3aH- cyclopenta[b]furan-3,3a-diol | 469 (M + 1) |
| 21 | 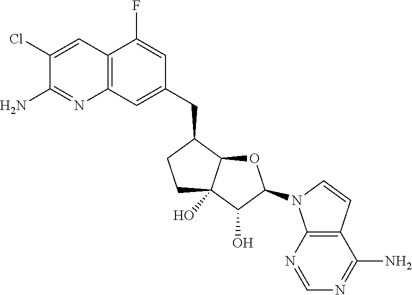 [SMILES: Nc1nc2cc(C[C@@H]3CC[C@@]4(O)[C@@H]3O[C@@H](n3ccc5c(N)ncnc53)[C@@H]4O)cc(F)c2cc1Cl] | (2R,3R,3aS,6S,6aR)-6-[(2-amino- 3-chloro-5-fluoroquinolin-7- yl)methyl]-2-(4-amino-7H- pyrrolo[2,3-d]pyrimidin-7- yl)hexahydro-3aH- cyclopenta[b]furan-3,3a-diol | 485 (M + 1) |
| 22 | 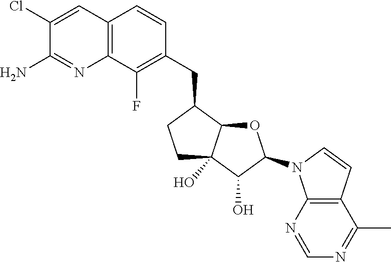 [SMILES: Cc1ncnc2c1ccn2[C@@H]1O[C@@H]2[C@H](Cc3ccc4cc(Cl)c(N)nc4c3F)CC[C@]2(O)[C@H]1O] | (2R,3R,3aS,6S,6aR)-6-[(2-amino- 3-chloro-8-fluoroquinolin-7- yl)methyl]-2-(4-methyl-7H- pyrrolo[2,3-d]pyrimidin-7- yl)hexahydro-3aH- cyclopenta[b]furan-3,3a-diol | 484 (M + 1) |
| 23 | 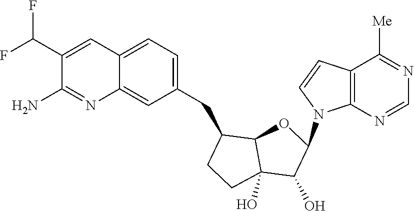 [SMILES: Cc1ncnc2c1ccn2[C@@H]1O[C@@H]2[C@H](Cc3ccc4cc(C(F)F)c(N)nc4c3)CC[C@]2(O)[C@H]1O] | (2R,3R,3aS,6S,6aR)-6-((2-amino- 3-(difluoromethyl)quinolin-7- yl)methyl)-2-(4-methyl-7H- pyrrolo[2,3-d]pyrimidin-7- yl)hexahydro-3aH- cyclopenta[b]furan-3,3a-diol | 482 (M + 1) |
| 24 | 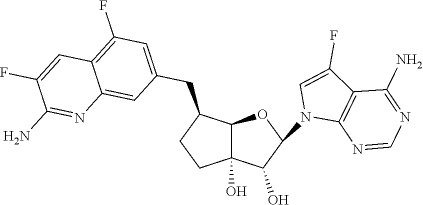 [SMILES: Nc1nc2cc(C[C@@H]3CC[C@@]4(O)[C@@H]3O[C@@H](n3cc(F)c5c(N)ncnc53)[C@@H]4O)cc(F)c2cc1F] | (2R,3R,3aS,6S,6aR)-6-((2-amino- 3,5-difluoroquinolin-7- yl)methyl)-2-(4-amino-5-fluoro- 7H-pyrrolo[2,3-d]pyrimidin-7- yl)hexahydro-3aH- cyclopenta[b]furan-3,3a-diol | 487 (M + 1) |

### Example 25

### (2R,3R,3aS,6S,6aR)-6-((6-amino-7-fluoro-1,5-naphthyridin-3-yl)methyl)-2-(4-methyl-7H-pyrrolo[2,3-d]pyrimidin-7-yl)hexahydro-3aH-cyclopenta[b]furan-3,3a-diol, 3HCl

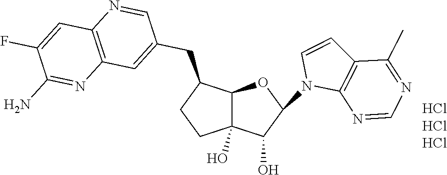 [SMILES: Cc1ncnc2c1ccn2[C@@H]1O[C@@H]2[C@H](Cc3cnc4cc(F)c(N)nc4c3)CC[C@]2(O)[C@H]1O]

Step 1: To a vial containing 7-((3aR,4R,5aR,8aR)-2,2-dimethyl-6-methylenehexahydrocyclopenta[2,3]furo[3,4-d][1,3]dioxol-4-yl)-4-methyl-7H-pyrrolo[2,3-d]pyrimidine (0.070 g, 0.214 mmol) was added THF (2.047 mL), followed by 9-BBN (0.5 M in THF, 1.37 mL, 0.684 mmol). The reaction was stirred at room temperature, under an argon atmosphere, overnight. Then aqueous tripotassium phosphate (1 M, 1.155 mL, 1.155 mmol) was added, and the reaction was vigorously stirred at room temperature for 1 h. Then a solution of 7-bromo-3-fluoro-1,5-naphthyridin-2-amine (0.078 g, 0.321 mmol) and [1,1′-bis(diphenylphosphino)ferrocene]dichloropalladium(II) (0.016 g, 0.021 mmol) in THF (1.01 mL) was added, and the reaction was heated to 50° C. for 2 h. The reaction was cooled to room temperature and poured into a separatory funnel containing water and EtOAc. After extraction, the aqueous layer was washed with EtOAc (×2). The organic layers were combined, dried over Na₂SO₄, filtered, and concentrated under reduced pressure. The material was purified by mass triggered reverse phase HPLC (ACN/water with 0.1% TFA for the modifier) to afford 7-(((3aR,4R,5aR,6S,8aR)-2,2-dimethyl-4-(4-methyl-7H-pyrrolo[2,3-d]pyrimidin-7-yl)hexahydrocyclopenta[2,3]furo[3,4-d][1,3]dioxol-6-yl)methyl)-3-fluoro-1,5-naphthyridin-2-amine as the TFA salt. MS: 491 (M+1).

Step 2: To a solution of 7-(((3aR,4R,5aR,6S,8aR)-2,2-dimethyl-4-(4-methyl-7H-pyrrolo[2,3-d]pyrimidin-7-yl)hexahydrocyclopenta[2,3]furo[3,4-d][1,3]dioxol-6-yl)methyl)-3-fluoro-1,5-naphthyridin-2-amine (0.117 g, 0.239 mmol) in MeOH (11.9 mL) was added dropwise hydrochloric acid (2 M, 11.9 mL, 23.9 mmol). The reaction was stirred for 90 minutes at room temperature. The temperature was increased to 40° C. and allowed to stir for 18 h. The reaction was split in two separate vials and concentrated under reduced pressure to afford (2R,3R,3aS,6S,6aR)-6-((6-amino-7-fluoro-1,5-naphthyridin-3-yl)methyl)-2-(4-methyl-7H-pyrrolo[2,3-d]pyrimidin-7-yl)hexahydro-3aH-cyclopenta[b]furan-3,3a-diol, 3HCl. MS: 451 (M+1). ¹H NMR (499 MHz, DMSO-d₆) δ 9.19 (s, 1H), 8.66 (d, J=1.4 Hz, 1H), 8.34 (d, J=3.8 Hz, 1H), 8.20 (d, J=10.8 Hz, 1H), 8.10 (s, 1H), 7.32 (d, J=3.8 Hz, 1H), 6.12 (d, J=8.2 Hz, 1H), 4.21 (d, J=8.2 Hz, 1H), 4.10 (d, J=5.9 Hz, 1H), 3.02-2.91 (m, 3H), 2.87-2.80 (m, 1H), 2.45-2.35 (m, 1H), 2.05-1.95 (m, 1H), 1.89-1.79 (m, 1H), 1.78-1.70 (m, 1H), 1.62-1.52 (m, 1H).

Examples 26-29: Examples 26-29 in Table 10 were synthesized in an analogously to steps 1-2 of Example 25 by substituting the 7-((3aS,4R,5aR,8aR)-2,2-dimethyl-6-methylenehexahydro-5H-pentaleno[1,6a-d][1,3]dioxol-4-yl)-4-methyl-7H-pyrrolo[2,3-d]pyrimidine with an appropriate exo-olefin and 7-bromo-3-fluoroquinolin-2-amine with an appropriate aryl-halide and followed by chiral resolution by SFC if needed based on the substituted exo-olefin.

TABLE 10

| Ex | Structure | Name | MS |
|---|---|---|---|
| 26 | 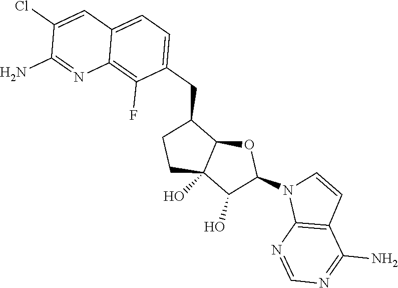 [SMILES: Nc1nc2c(F)c(C[C@@H]3CC[C@@]4(O)[C@@H]3O[C@@H](n3ccc5c(N)ncnc53)[C@@H]4O)ccc2cc1Cl] | (2R,3R,3aS,6S,6aR)-6-((2- amino-3-chloro-8-fluoroquinolin- 7-yl)methyl)-2-(4-amino-7H- pyrrolo[2,3-d]pyrimidin-7- yl)hexahydro-3aH- cyclopenta[b]furan-3,3a-diol | 485 (M + 1) |
| 27 | 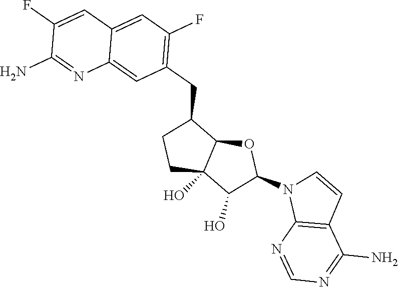 [SMILES: Nc1nc2cc(C[C@@H]3CC[C@@]4(O)[C@@H]3O[C@@H](n3ccc5c(N)ncnc53)[C@@H]4O)c(F)cc2cc1F] | (2R,3R,3aS,6S,6aR)-6-[(2-amino- 3,6-difluoroquinolin-7- yl)methyl]-2-(4-amino-7H- pyrrolo[2,3-d]pyrimidin-7- yl)hexahydro-3aH- cyclopenta[b]furan-3,3a-diol | 469 (M + 1) |
| 28 | 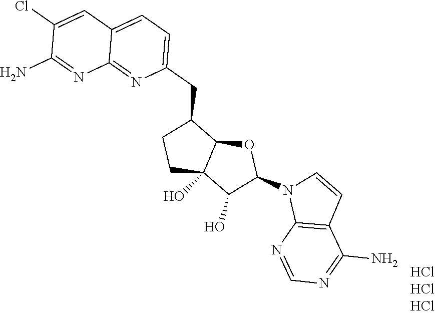 [SMILES: Nc1nc2nc(C[C@@H]3CC[C@@]4(O)[C@@H]3O[C@@H](n3ccc5c(N)ncnc53)[C@@H]4O)ccc2cc1Cl] | (2R,3R,3aS,6S,6aR)-6-((7- amino-6-chloro-1,8- naphthyridin-2-yl)methyl)-2-(4- amino-7H-pyrrolo[2,3- d]pyrimidin-7-yl)hexahydro- 3aH-cyclopenta[b]furan-3,3a-diol trihydrochloride | 468 (M + 1) |
| 29 | 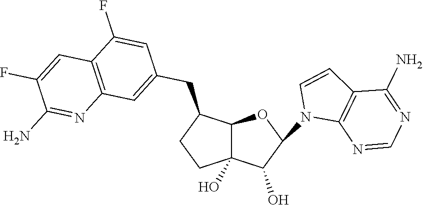 [SMILES: Nc1nc2cc(C[C@@H]3CC[C@@]4(O)[C@@H]3O[C@@H](n3ccc5c(N)ncnc53)[C@@H]4O)cc(F)c2cc1F] | (2R,3R,3aS,6S,6aR)-6-[(2-amino- 3,5-difluoroquinolin-7- yl)methyl]-2-(4-amino-7H- pyrrolo[2,3-d]pyrimidin-7- yl)hexahydro-3aH- cyclopenta[b]furan-3,3a-diol | 469 (M + 1) |

### Example 30

### (1S,2R,3aR,4S,6aR)-4-((2-amino-3-fluoroquinolin-7-yl)methyl)-2-(4-methyl-7H-pyrrolo[2,3-d]pyrimidin-7-yl)hexahydropentalene-1,6a(1H)-diol

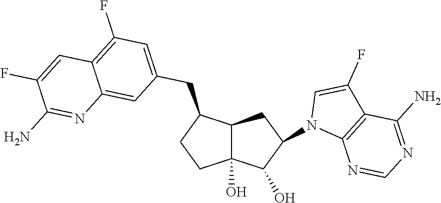 [SMILES: Nc1nc2cc(C[C@@H]3CC[C@@]4(O)[C@@H]3C[C@@H](n3cc(F)c5c(N)ncnc53)[C@@H]4O)cc(F)c2cc1F]

Step 1: To a flask containing 1,1′-bis(diphenylphosphino)ferrocene (1.6 g, 2.8 mmol), sodium tert-butoxide (0.96 g, 8.4 mmol), 4-chloro-5-fluoro-7H-pyrrolo[2,3-d]pyrimidine (1.4 g, 8.4 mmol), and allyl palladium chloride dimer (0.42 g, 1.1 mmol) was added argon-degassed THF (55 mL). This mixture was stirred at room temperature for 15 minutes. Then a solution of (2R,6S,6aS)-6-((triphenylsilyl)oxy)-1,2,4,5,6,6a-hexahydropentalen-2-yl acetate (2.47 g, 5.61 mmol) in degassed THF (23 mL) was added. The reaction was stirred under an argon atmosphere at 40° C. for 3 h. The mixture was quenched with water and extracted with EtOAc (2×). The combined organic layers were washed with brine, dried over anhydrous sodium sulfate, filtered, and concentrated under reduced pressure. The residue was purified by column chromatography on silica (0-15% EtOAc/hexanes) to afford 4-chloro-5-fluoro-7-((2R,6S,6aS)-6-((triphenylsilyl)-oxy)-1,2,4,5,6,6a-hexahydropentalen-2-yl)-7H-pyrrolo[2,3-d]pyrimidine. MS: 552 (M+1).

Step 2: To a flask containing a solution of 4-chloro-5-fluoro-7-((2R,6S,6aS)-6-((triphenylsilyl)oxy)-1,2,4,5,6,6a-hexahydropentalen-2-yl)-7H-pyrrolo[2,3-d]pyrimidine (1.36 g, 2.46 mmol) in THF (59 mL) was added water (29 mL). The solution was cooled to 0° C., and NMO (0.58 g, 4.9 mmol) and osmium (VIII) oxide (0.75 mL, 4% in water, 0.12 mmol) were added. The ice bath was allowed to naturally expire as the reaction was stirred overnight. The reaction was quenched with saturated aqueous sodium sulfite (40 mL), and the mixture was stirred at room temperature for 15 minutes. The reaction was poured into a separatory funnel containing water and EtOAc. After separation, the aqueous layer was washed twice with EtOAc and then once with 3:1 chloroform:IPA. The combined organic layers were dried over anhydrous sodium sulfate, filtered, and concentrated under reduced pressure to afford (1S,2R,3aR,4S,6aR)-2-(4-chloro-5-fluoro-7H-pyrrolo[2,3-d]pyrimidin-7-yl)-4-((triphenylsilyl)oxy)hexahydro-pentalene-1,6a(1H)-diol. The material was used crude directly in the next step without further purification.

Step 3: To a flask containing (1S,2R,3aR,4S,6aR)-2-(4-chloro-5-fluoro-7H-pyrrolo[2,3-d]pyrimidin-7-yl)-4-((triphenylsilyl)oxy)hexahydropentalene-1,6a(1H)-diol (1.4 g, 2.4 mmol) dissolved in acetone (24 mL) under an argon atmosphere was added sulfuric acid (0.126 mL, 2.36 mmol). The mixture was stirred at room temperature for 4 h. The reaction was cooled to 0° C., and quenched with sodium hydroxide (0.3 mL, 10 M in water, 3 mmol). The material was diluted with water (20 mL) and extracted with EtOAc (3×), and the combined organic layers were dried over anhydrous sodium sulfate, filtered, and concentrated under reduced pressure. The residue was purified by column chromatography on silica (50% EtOAc/hexanes) to afford (3aS,4R,5aR,6S,8aR)-4-(4-chloro-5-fluoro-7H-pyrrolo[2,3-d]pyrimidin-7-yl)-2,2-dimethylhexahydro-5H-pentaleno[1,6a-d][1,3]dioxol-6-ol. MS: 368 (M+1). ¹H NMR (600 MHz, CDCl₃) δ 8.60 (s, 1H), 7.05 (d, J=2.6 Hz, 1H), 4.91-4.85 (m, 1H), 4.70 (d, J=7.2 Hz, 1H), 4.11-4.08 (m, 1H), 2.59-2.52 (m, 2H), 2.41-2.34 (m, 1H), 2.25-2.19 (m, 1H), 2.13-2.06 (m, 1H), 2.06-1.93 (m, 2H), 1.59 (s, 3H), 1.38 (s, 3H).

Step 4: To a solution of (3aS,4R,5aR,6S,8aR)-4-(4-chloro-5-fluoro-7H-pyrrolo[2,3-d]pyrimidin-7-yl)-2,2-dimethylhexahydro-5H-pentaleno[1,6a-d][1,3]dioxol-6-ol (270 mg, 0.734 mmol) in anhydrous DCM (5 mL) at 0° C. under a nitrogen atmosphere was added Dess-Martin Periodinane (374 mg, 0.881 mmol) in one portion. The mixture was stirred at room temperature overnight. The reaction was quenched with saturated aqueous sodium bicarbonate (10 mL) and 1 g of Na₂S₂O₃. The resulting mixture was stirred for 10 minutes at room temperature. The organic layer was separated, and the aqueous phase was extracted with DCM (10 mL×3). The combined organic layers were washed with brine (10 mL), dried with anhydrous sodium sulfate, filtered, and concentrated under reduced pressure. The residue was purified by column chromatography on silica (0-60% EtOAc/hexanes) to afford (3aS,4R,5aS,8aR)-4-(4-chloro-5-fluoro-7H-pyrrolo[2,3-d]pyrimidin-7-yl)-2,2-dimethylhexahydro-6H-pentaleno[1,6a-d][1,3]dioxol-6-one. MS: 366 (M+1).

Step 5: To a vial was added methyltriphenylphosphonium bromide (781 mg, 2.14 mmol), followed by THF (3.3 mL) under an argon atmosphere. The mixture was cooled to 0° C., and then nBuLi (650 μl, 2.5 M, 1.624 mmol) was added dropwise. The reaction was brought out of the ice bath and allowed to vigorously stir at room temperature for 30 minutes. Then the reaction was cooled back down to 0° C., and a solution of (3aS,4R,5aS,8aR)-4-(4-chloro-5-fluoro-7H-pyrrolo[2,3-d]pyrimidin-7-yl)-2,2-dimethylhexahydro-6H-pentaleno[1,6a-d][1,3]dioxol-6-one (238 mg, 0.650 mmol) in THF (3.3 mL) was added dropwise. The reaction was brought out of the bath and allowed to stir at room temperature for 70 minutes. The reaction was quenched with saturated aqueous ammonium chloride (10 mL) at 0° C. The mixture was extracted with EtOAc (2×10 mL), and the combined organic layers were washed with brine, dried over anhydrous sodium sulfate, filtered, and concentrated under reduced pressure. The residue was purified by column chromatography on silica (0-50% EtOAc/hexanes) to afford 4-chloro-7-((3aS,4R,5aR,8aR)-2,2-dimethyl-6-methylenehexahydro-5H-pentaleno[1,6a-d][1,3]dioxol-4-yl)-5-fluoro-7H-pyrrolo[2,3-d]pyrimidine. MS: 364 (M+1).

Step 6: To an argon-purged vial containing 4-chloro-7-((3aS,4R,5aR,8aR)-2,2-dimethyl-6-methylenehexahydro-5H-pentaleno[1,6a-d][1,3]dioxol-4-yl)-5-fluoro-7H-pyrrolo[2,3-d]pyrimidine (201.6 mg, 0.554 mmol) was added THF (4 ml). Then 9-BBN (3.4 ml, 1.700 mmol, 0.5 M in THF) solution was added. The reaction was stirred overnight at room temperature under a balloon of argon. Then tripotassium phosphate (1.4 ml, 2.80 mmol, 2 M aqueous) was added, and the reaction was vigorously stirred for −35 min. Then potassium tert-butoxide (0.1 ml, 0.100 mmol) was added, followed quickly by a solution of chloro[di(1-adamantyl)-N-butylphosphine)-2-(2-aminobiphenyl)]palladium(II) (60 mg, 0.085 mmol) and 7-bromo-3,5-difluoroquinolin-2-amine (215 mg, 0.831 mmol) in THF (4 ml). The reaction was heated to 50 degrees under argon for 4 hrs. 40 min. The reaction was cooled to room temperature, and diluted with DCM and water. The mixture was passed through a phase separator. The organic layer was concentrated under reduced pressure, and the crude material was subjected to silica gel flash chromatography (20-40-60% EtOAc/hexanes) followed by chiral SFC (OJ-H column, 15% MeOH w/0.1% NH₄OH modifier in CO2) to afford 7-(((3aS,4R,5aR,6S,8aR)-4-(4-chloro-5-fluoro-7H-pyrrolo[2,3-d]pyrimidin-7-yl)-2,2-dimethylhexahydro-5H-pentaleno[1,6a-d][1,3]dioxol-6-yl)methyl)-3,5-difluoroquinolin-2-amine as a foam.

Step 7: To a vial containing 7-(((3aS,4R,5aR,6S,8aR)-4-(4-chloro-5-fluoro-7H-pyrrolo[2,3-d]pyrimidin-7-yl)-2,2-dimethylhexahydro-5H-pentaleno[1,6a-d][1,3]dioxol-6-yl)methyl)-3,5-difluoroquinolin-2-amine (21.4 mg, 0.039 mmol) was added ammonia (1 mL, 7 M in MeOH, 7 mmol). The vial was capped, and the reaction was heated at 140° C. in a microwave reactor for 5 h. The mixture was then concentrated under reduced pressure to give 7-(((3aS,4R,5aR,6S,8aR)-4-(4-amino-5-fluoro-7H-pyrrolo[2,3-d]pyrimidin-7-yl)-2,2-dimethylhexahydro-5H-pentaleno[1,6a-d][1,3]dioxol-6-yl)methyl)-3,5-difluoroquinolin-2-amine, which was used crude without further purification in the next reaction.

Step 8: To a vial containing 7-(((3aS,4R,5aR,6S,8aR)-4-(4-amino-5-fluoro-7H-pyrrolo[2,3-d]pyrimidin-7-yl)-2,2-dimethylhexahydro-5H-pentaleno[1,6a-d][1,3]dioxol-6-yl)methyl)-3,5-difluoroquinolin-2-amine (21 mg, 0.039 mmol) was added DCM (600 μL), water (190 μL), and then TFA (454 μL, 5.89 mmol). The mixture was stirred at room temperature for 3 h. The reaction was directly concentrated under reduced pressure. The residue was purified by mass-triggered reverse phase HPLC (ACN/water gradient with 0.1% NH₄OH modifier) to afford (1S,2R,3aR,4S,6aR)-4-((2-amino-3,5-difluoroquinolin-7-yl)methyl)-2-(4-amino-5-fluoro-7H-pyrrolo[2,3-d]pyrimidin-7-yl)hexahydropentalene-1,6a(1H)-diol. MS: 485 (M+1). ¹H NMR (600 MHz, DMSO) δ 8.69 (s, 2H), 8.29 (s, 1H), 7.98 (d, J=11.1 Hz, 1H), 7.87 (s, 1H), 7.68 (s, 2H), 7.19 (s, 1H), 7.06 (d, J=11.0 Hz, 1H), 4.84-4.77 (m, 1H), 3.76 (d, J=10.3 Hz, 1H), 2.79-2.66 (m, 2H), 2.49-2.42 (m, 1H), 2.16 (q, J=9.0 Hz, 1H), 1.82 (dd, J=12.2, 5.8 Hz, 1H), 1.78-1.69 (m, 2H), 1.69-1.63 (m, 1H), 1.63-1.54 (m, 1H), 1.48-1.41 (m, 1H).

Examples 31-34: Examples 31-34 in Table 11 were synthesized in an analogously to Example 30 by substituting 4-chloro-5-fluoro-7H-pyrrolo[2,3-d]pyrimidine with an appropriate nucleobase in step 1 and step 6 is replaced with Step 3 in Example 16, where an appropriate aryl-halide substitutes 7-bromo-3,5-difluoroquinolin-2-amine.

TABLE 11

| Ex | Structure | Name | MS |
|---|---|---|---|
| 31 | 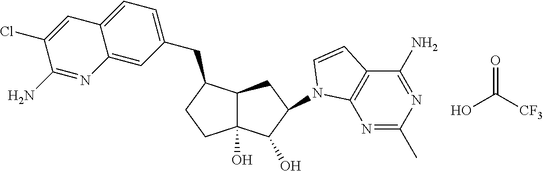 [SMILES: Cc1nc(N)c2ccn([C@@H]3C[C@@H]4[C@H](Cc5ccc6cc(Cl)c(N)nc6c5)CC[C@]4(O)[C@H]3O)c2n1 ; O=C(O)C(F)(F)F] | (1S,2R,3aR,4S,6aR)-2-(4- amino-2-methyl-7H- pyrrolo[2,3-d]pyrimidin-7-yl)- 4-((2-amino-3-chloroquinolin- 7-yl)methyl) hexahydropentalene- 1,6a(1H)-diol 2,2,2- trifluoroacetate | 479 (M + 1) |
| 32 | 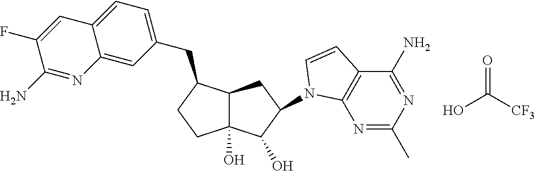 [SMILES: Cc1nc(N)c2ccn([C@@H]3C[C@@H]4[C@H](Cc5ccc6cc(F)c(N)nc6c5)CC[C@]4(O)[C@H]3O)c2n1 ; O=C(O)C(F)(F)F] | (1S,2R,3aR,4S,6aR)-2-(4- amino-2-methyl-7H- pyrrolo[2,3-d]pyrimidin-7-yl)- 4-((2-amino-3-fluoroquinolin- 7-yl)methyl) hexahydropentalene- 1,6a(1H)-diol 2,2,2- trifluoroacetate | 463 (M + 1) |
| 33 | 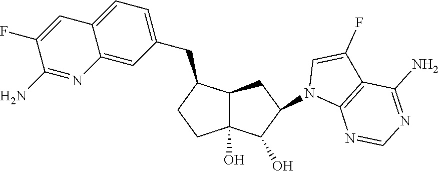 [SMILES: Nc1nc2cc(C[C@@H]3CC[C@@]4(O)[C@@H]3C[C@@H](n3cc(F)c5c(N)ncnc53)[C@@H]4O)ccc2cc1F] | (1S,2R,3aR,4S,6aR)-4-((2- amino-3-fluoroquinolin-7- yl)methyl)-2-(4-amino-5- fluoro-7H-pyrrolo[2,3- d]pyrimidin-7- yl)hexahydropentalene- 1,6a(1H)-diol | 467 (M + 1) |
| 34 | 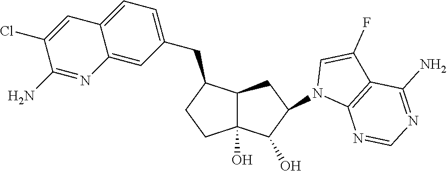 [SMILES: Nc1nc2cc(C[C@@H]3CC[C@@]4(O)[C@@H]3C[C@@H](n3cc(F)c5c(N)ncnc53)[C@@H]4O)ccc2cc1Cl] | (1S,2R,3aR,4S,6aR)-4-((2- amino-3-chloroquinolin-7- yl)methyl)-2-(4-amino-5- fluoro-7H-pyrrolo[2,3- d]pyrimidin-7- yl)hexahydropentalene- 1,6a(1H)-diol | 483 (M + 1) |

Examples 35-42: Examples 35-42 in Table 12 were synthesized in an analogously Example 30 by substituting 4-chloro-5-fluoro-7H-pyrrolo[2,3-d]pyrimidine with an appropriate nucleobase in step 1 and step 6 is replaced with Step 1 in Example 25, where an appropriate aryl-halide substitutes 7-bromo-3,5-difluoroquinolin-2-amine.

TABLE 12

| Ex | Structure | Name | MS |
|---|---|---|---|
| 35 | 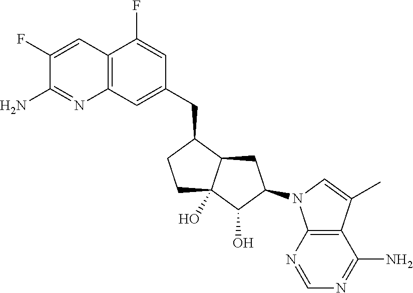 [SMILES: Cc1cn([C@@H]2C[C@@H]3[C@H](Cc4cc(F)c5cc(F)c(N)nc5c4)CC[C@]3(O)[C@H]2O)c2ncnc(N)c12] | (1S,2R,3aR,4S,6aR)-4- [(2-amino-3,5-difluoroquinolin- 7-yl)methyl]-2-(4-amino-5- methyl-7H-pyrrolo[2,3-d] pyrimidin-7-yl) hexahydropentalene-1,6a(1H)- diol | 481 (M + 1) |
| 36 | 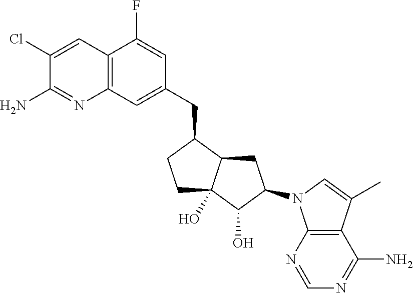 [SMILES: Cc1cn([C@@H]2C[C@@H]3[C@H](Cc4cc(F)c5cc(Cl)c(N)nc5c4)CC[C@]3(O)[C@H]2O)c2ncnc(N)c12] | (1S,2R,3aR,4S,6aR)-4-[(2-amino- 3-chloro-5-fluoroquinolin-7- yl)methyl]-2-(4-amino-5-methyl- 7H-pyrrolo[2,3-d]pyrimidin-7- yl)hexahydropentalene-1,6a(1H)- diol | 497 (M + 1) |
| 37 | 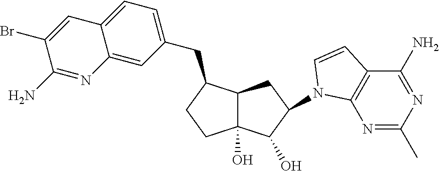 [SMILES: Cc1nc(N)c2ccn([C@@H]3C[C@@H]4[C@H](Cc5ccc6cc(Br)c(N)nc6c5)CC[C@]4(O)[C@H]3O)c2n1] | (1S,2R,3aR,4S,6aR)-2- (4-amino- 2-methyl-7H-pyrrolo[2,3- d]pyrimidin-7-yl)-4- ((2-amino-3-bromoquinolin-7- yl)methyl)hexahydropentalene- 1,6a(1H)-diol | 523/525 (M + 1/ M + 3) |
| 38 | 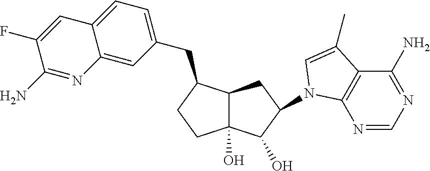 [SMILES: Cc1cn([C@@H]2C[C@@H]3[C@H](Cc4ccc5cc(F)c(N)nc5c4)CC[C@]3(O)[C@H]2O)c2ncnc(N)c12] | (1S,2R,3aR,4S,6aR)-4-((2- amino-3-fluoroquinolin-7- yl)methyl)-2-(4-amino-5-methyl- 7H-pyrrolo[2,3-d]pyrimidin-7- yl)hexahydropentalene- 1,6a(1H)-diol | 463 (M + 1) |
| 39 | 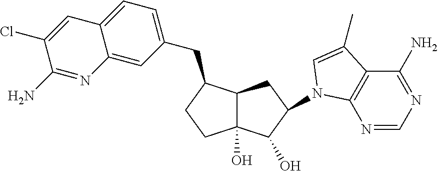 [SMILES: Cc1cn([C@@H]2C[C@@H]3[C@H](Cc4ccc5cc(Cl)c(N)nc5c4)CC[C@]3(O)[C@H]2O)c2ncnc(N)c12] | (1S,2R,3aR,4S,6aR)-4-((2- amino-3-chloroquinolin-7- yl)methyl)-2-(4-amino-5-methyl- 7H-pyrrolo[2,3-d]pyrimidin-7- yl)hexahydropentalene- 1,6a(1H)-diol | 479 (M + 1) |
| 40 | 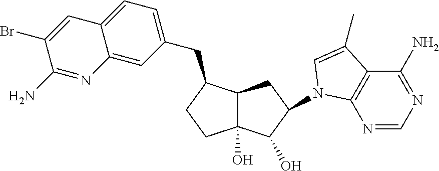 [SMILES: Cc1cn([C@@H]2C[C@@H]3[C@H](Cc4ccc5cc(Br)c(N)nc5c4)CC[C@]3(O)[C@H]2O)c2ncnc(N)c12] | (1S,2R,3aR,4S,6aR)-4-((2- amino-3-bromoquinolin-7- yl)methyl)-2-(4-amino-5-methyl- 7H-pyrrolo[2,3-d]pyrimidin-7- yl)hexahydropentalene- 1,6a(1H)-diol | 523/525 (M + 1/ M + 3) |
| 41 | 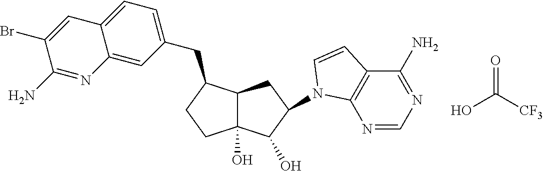 [SMILES: Nc1nc2cc(C[C@@H]3CC[C@@]4(O)[C@@H]3C[C@@H](n3ccc5c(N)ncnc53)[C@@H]4O)ccc2cc1Br ; O=C(O)C(F)(F)F] | (1S,2R,3aR,4S,6aR)-4-((2- amino-3-bromoquinolin-7- yl)methyl)-2-(4-amino-7H- pyrrolo[2,3-d]pyrimidin-7- yl)hexahydropentalene- 1,6a(1H)- diol 2,2,2-trifluoroacetate | 509/511 (M + 1/ M + 3) |
| 42 | 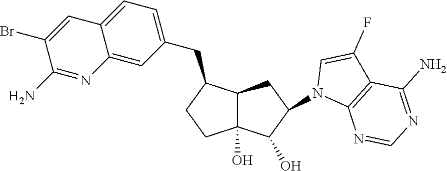 [SMILES: Nc1nc2cc(C[C@@H]3CC[C@@]4(O)[C@@H]3C[C@@H](n3cc(F)c5c(N)ncnc53)[C@@H]4O)ccc2cc1Br] | (1S,2R,3aR,4S,6aR)-4-((2- amino-3-bromoquinolin-7- yl)methyl)-2-(4-amino-5-fluoro- 7H-pyrrolo[2,3-d]pyrimidin-7- yl)hexahydropentalene- 1,6a(1H)-diol | 527/529 (M + 1/ M + 3) |

Examples 43-44: Examples 43-44 in Table 13 were synthesized by applying the protocols as described in steps 6-8 of Example 30 by substituting 7-bromo-3,5-difluoroquinolin-2-amine with an appropriate aryl-halide in step 6.

TABLE 13

| Ex | Structure | Name | MS |
|---|---|---|---|
| 43 | 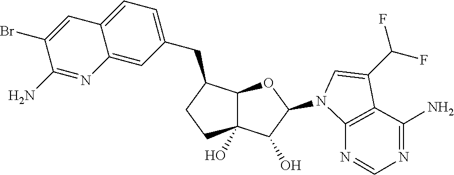 [SMILES: Nc1nc2cc(C[C@@H]3CC[C@@]4(O)[C@@H]3O[C@@H](n3cc(C(F)F)c5c(N)ncnc53)[C@@H]4O)ccc2cc1Br] | (2R,3R,3aS,6S,6aR)- 6-[(2-amino-3- bromoquinolin-7- yl)methyl]-2-[4- amino-5- (difluoromethyl)-7H- pyrrolo[2,3- d]pyrimidin-7- yl]hexahydro-3aH- cyclopenta[b]furan- 3,3a-diol | 561/563 (M + 1/M + 3) |
| 44 | 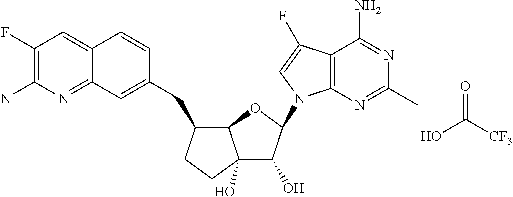 | (2R,3R,3aS,6S,6aR)- 6-((2-amino-3- fluoroquinolin-7- yl)methyl)-2-(4- amino-5-fluoro-2- methyl-7H-pyrrolo [2,3-d]pyrimidin-7- yl)hexahydro-3aH- cyclopenta[b]furan- 3,3a-diol 2,2,2- trifluoroacetate | 484 (M + 1) |

### Example 45

### (2R,3R,3aS,6S,6aR)-2-(4-amino-2-fluoro-7H-pyrrolo[2,3-d]pyrimidin-7-yl)-6-((2-amino-3-fluoroquinolin-7-yl)methyl)hexahydro-3aH-cyclopenta[b]furan-3,3a-diol

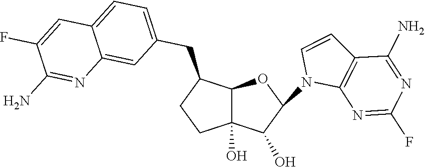 [SMILES: Nc1nc2cc(C[C@@H]3CC[C@@]4(O)[C@@H]3O[C@@H](n3ccc5c(N)nc(F)nc53)[C@@H]4O)ccc2cc1F]

Step 1: To 2-fluoro-7H-pyrrolo[2,3-d]pyrimidin-4-amine (500 mg, 3.29 mmol) dissolved in DCM (8 mL) and acetonitrile (8 mL) was added di-tert-butyl dicarbonate (2.5 g, 12 mmol) and DMAP (80 mg, 0.66 mmol). The solution was stirred at room temperature for 1 h. The mixture was then concentrated under reduced pressure, and the residue was purified by column chromatography on silica (0-30% EtOAc/hexanes) to afford tert-butyl 4-[bis(tert-butoxycarbonyl)amino]-2-fluoro-7H-pyrrolo[2,3-d]pyrimidine-7-carboxylate. MS: 453 (M+1).

¹H NMR (600 MHz, CDCl₃) δ 7.64 (d, J=4.1 Hz, 1H), 6.48 (d, J=4.1 Hz, 1H), 1.69 (s, 9H), 1.44 (s, 18H).

Step 2: To a solution of tert-butyl 4-[bis(tert-butoxycarbonyl)amino]-2-fluoro-7H-pyrrolo[2,3-d]pyrimidine-7-carboxylate (1.35 g, 2.98 mmol) in MeOH (15 mL) was added TEA (4.15 mL, 29.8 mmol) at room temperature. The reaction was heated to 60° C. and stirred overnight. The mixture was cooled to room temperature and concentrated under reduced pressure. The residue was purified by column chromatography on silica (0-60% EtOAc/hexanes) to afford di-tert-butyl (2-fluoro-7H-pyrrolo[2,3-d]pyrimidin-4-yl)imidodicarbonate. MS: 353 (M+1). ¹H NMR (600 MHz, CDCl₃) δ 9.34 (s, 1H), 7.28 (dd, J=3.5, 2.3 Hz, 1H), 6.50 (dd, J=3.6, 2.0 Hz, 1H), 1.47 (s, 18H).

Step 3: To a vial containing (3R,3aS,6S,6aR)-6-((2-amino-3-fluoroquinolin-7-yl)methyl)hexahydro-3aH-cyclopenta[b]furan-2,3,3a-triol (100 mg, 0.299 mmol) dissolved in dry acetonitrile (5 mL) was added 1,1′-(azodicarbonyl)dipiperidine (113 mg, 0.45 mmol) followed by tri-n-butylphosphine (120 μL, 0.48 mmol) at room temperature. The mixture was stirred for 1 h. In a separate vial containing di-tert-butyl (2-fluoro-7H-pyrrolo[2,3-d]pyrimidin-4-yl)imidodicarbonate (211 mg, 0.598 mmol) dissolved in dry acetonitrile (1 mL) was added DBU (90 μL, 0.60 mmol). The mixture was stirred for 30 minutes at room temperature. The mixture was then added to the mixture containing the pre-formed epoxide, and the reaction was stirred for 2.5 h at room temperature. The mixture was then filtered and purified by mass-triggered reverse phase HPLC (ACN/water with 0.1% NH₄OH modifier) to afford di-tert-butyl (7-{(2R,3R,3aS,6S,6aR)-6-[(2-amino-3-fluoroquinolin-7-yl)methyl]-3,3a-dihydroxyhexahydro-2H-cyclopenta[b]furan-2-yl}-2-fluoro-7H-pyrrolo[2,3-d]pyrimidin-4-yl)imidodicarbonate. MS: 669 (M+1).

Step 4: To di-tert-butyl (7-{(2R,3R,3aS,6S,6aR)-6-[(2-amino-3-fluoroquinolin-7-yl)methyl]-3,3a-dihydroxyhexahydro-2H-cyclopenta[b]furan-2-yl}-2-fluoro-7H-pyrrolo[2,3-d]pyrimidin-4-yl)imidodicarbonate (97 mg, 0.14 mmol) dissolved in DCM (3 mL) was added TFA (1.1 mL, 14.5 mmol). The mixture was stirred overnight at room temperature. The reaction was concentrated under reduced pressure. The residue was purified by mass-triggered reverse phase HPLC (ACN/water gradient with 0.1% NH₄OH modifier) to afford (2R,3R,3aS,6S,6aR)-2-(4-amino-2-fluoro-7H-pyrrolo[2,3-d]pyrimidin-7-yl)-6-((2-amino-3-fluoroquinolin-7-yl)methyl)hexahydro-3aH-cyclopenta[b]furan-3,3a-diol. MS: 469 (M+1). ¹H NMR (600 MHz, DMSO) δ 7.75 (d, J=11.8 Hz, 1H), 7.59 (s, 2H), 7.54 (d, J=8.2 Hz, 1H), 7.40 (d, J=3.7 Hz, 1H), 7.30 (s, 1H), 7.09 (d, J=8.2 Hz, 1H), 6.78-6.62 (m, 3H), 5.72 (d, J=8.1 Hz, 1H), 5.26 (d, J=7.0 Hz, 1H), 5.08 (s, 1H), 4.09 (t, J=7.5 Hz, 1H), 3.96 (d, J=5.7 Hz, 1H), 2.82 (dd, J=13.6, 7.8 Hz, 1H), 2.62 (dd, J=13.6, 7.2 Hz, 1H), 2.28-2.20 (m, 1H), 1.92 (dd, J=12.7, 6.0 Hz, 1H), 1.76-1.64 (m, 2H), 1.55-1.48 (m, 1H).

Example 46: Example 46 in Table 14 was synthesized by applying the Mitsunobu protocol described in step 3 of the synthesis of Example 45 followed by application of the TFA deprotection described in step 4 of Example 45. Di-tert-butyl (2-fluoro-7H-pyrrolo[2,3-d]pyrimidin-4-yl)imidodicarbonate and (3R,3aS,6S,6aR)-6-((2-amino-3-fluoroquinolin-7-yl)methyl)hexahydro-3aH-cyclopenta[b]furan-2,3,3a-triol in the Mitsunobu sequence were substituted with an appropriate nucleobase and triol respectively.

TABLE 14

| Ex | Structure | Name | MS |
|---|---|---|---|
| 46 | 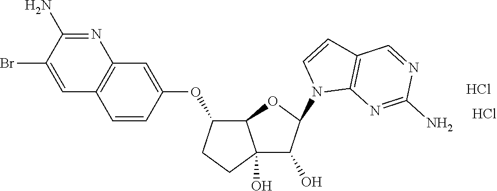 [SMILES: Nc1ncc2ccn([C@@H]3O[C@@H]4[C@@H](Oc5ccc6cc(Br)c(N)nc6c5)CC[C@]4(O)[C@H]3O)c2n1 ; Cl ; Cl] | (2R,3R,3aS,6S,6aR)-6-((2-amino- 3-bromoquinolin-7-yl)oxy)-2-(2- amino-7H-pyrrolo[2,3- d]pyrimidin-7-yl)hexahydro-3aH- cyclopenta[b]furan-3,3a-diol dihydrochloride | 513/515 (M + 1/ M + 3) |

Examples 47-48: Examples 47-48 in Table 15 were synthesized in an analogously to Example 45 by substituting 2-fluoro-7H-pyrrolo[2,3-d]pyrimidin-4-amine with an appropriate nucleobase in step 1 and (3R,3aS,6S,6aR)-6-((2-amino-3-fluoroquinolin-7-yl)methyl)hexahydro-3aH-cyclopenta[b]furan-2,3,3a-triol with an appropriate triol in step 3.

TABLE 15

| Ex | Structure | Name | LCMS |
|---|---|---|---|
| 47 | 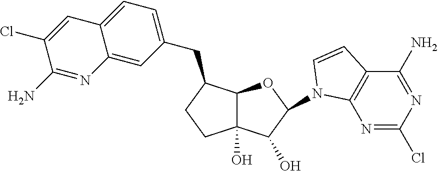 [SMILES: Nc1nc2cc(C[C@@H]3CC[C@@]4(O)[C@@H]3O[C@@H](n3ccc5c(N)nc(Cl)nc53)[C@@H]4O)ccc2cc1Cl] | (2R,3R,3aS,6S,6aR)-2-(4- amino-2-chloro-7H- pyrrolo[2,3-d]pyrimidin-7-yl)- 6-[(2-amino-3-chloroquinolin- 7-yl)methyl]hexahydro-3aH- cyclopenta[b]furan-3,3a-diol | 501 (M + 1) |
| 48 | 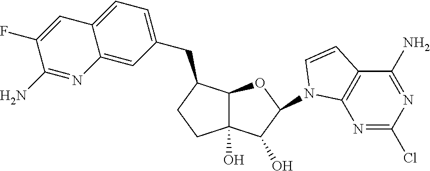 [SMILES: Nc1nc2cc(C[C@@H]3CC[C@@]4(O)[C@@H]3O[C@@H](n3ccc5c(N)nc(Cl)nc53)[C@@H]4O)ccc2cc1F] | (2R,3R,3aS,6S,6aR)-2-(4- amino-2-chloro-7H- pyrrolo[2,3-d]pyrimidin-7-yl)- 6-[(2-amino-3-fluoroquinolin- 7-yl)methyl]hexahydro-3aH- cyclopenta[b]furan-3,3a-diol | 485 (M + 1) |

Examples 49-66: Examples 49-66 in Table 16 were synthesized by applying the Mitsunobu protocol described in step 3 of the synthesis of Example 45 followed by application of the ammnolysis protocol described in step 7 of Example 30. Di-tert-butyl (2-fluoro-7H-pyrrolo[2,3-d]pyrimidin-4-yl)imidodicarbonate and (3R,3aS,6S,6aR)-6-((2-amino-3-fluoroquinolin-7-yl)methyl)hexahydro-3aH-cyclopenta[b]furan-2,3,3a-triol in the Mitsunobu sequence were substituted with an appropriate nucleobase and triol respectively.

TABLE 16

| Ex | Structure | Name | MS |
|---|---|---|---|
| 49 | 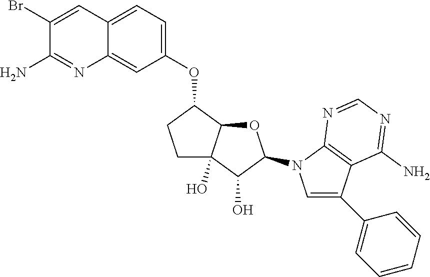 [SMILES: Nc1nc2cc(O[C@H]3CC[C@@]4(O)[C@@H]3O[C@@H](n3cc(-c5ccccc5)c5c(N)ncnc53)[C@@H]4O)ccc2cc1Br] | (2R,3R,3aS,6S,6aR)-6-[(2-amino-3- bromoquinolin-7-yl)oxy]-2-(4- amino-5-phenyl-7H-pyrrolo[2,3- d]pyrimidin-7-yl)hexahydro-3aH- cyclopenta[b]furan-3,3a-diol | 589/591 (M + 1/ M + 3) |
| 50 | 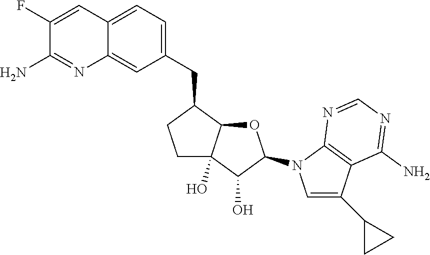 [SMILES: Nc1nc2cc(C[C@@H]3CC[C@@]4(O)[C@@H]3O[C@@H](n3cc(C5CC5)c5c(N)ncnc53)[C@@H]4O)ccc2cc1F] | (2R,3R,3aS,6S,6aR)-2-(4-amino-5- cyclopropyl-7H-pyrrolo[2,3- d]pyrimidin-7-yl)-6-[(2-amino-3- fluoroquinolin-7- yl)methyl]hexahydro-3aH- cyclopenta[b]furan-3,3a-diol | 491 (M + 1) |
| 51 | 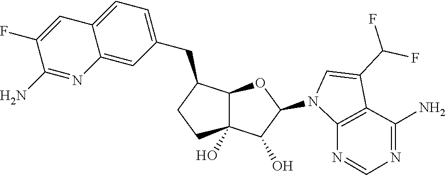 [SMILES: Nc1nc2cc(C[C@@H]3CC[C@@]4(O)[C@@H]3O[C@@H](n3cc(C(F)F)c5c(N)ncnc53)[C@@H]4O)ccc2cc1F] | (2R,3R,3aS,6S,6aR)-2-[4-amino-5- (difluoromethyl)-7H-pyrrolo[2,3- d]pyrimidin-7-yl]-6-[(2-amino-3- fluoroquinolin-7- yl)methyl]hexahydro-3aH- cyclopenta[b]furan-3,3a-diol | 501 (M + 1) |
| 52 | 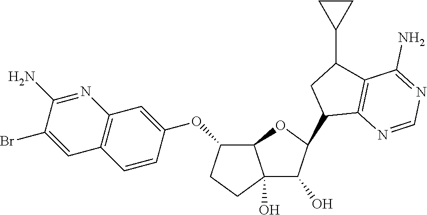 [SMILES: Nc1nc2cc(O[C@H]3CC[C@@]4(O)[C@@H]3O[C@@H](C3CC(C5CC5)c5c(N)ncnc53)[C@@H]4O)ccc2cc1Br] | (2R,3R,3aS,6S,6aR)-6-[(2-amino-3- bromoquinolin-7-yl)oxy]-2-(4- amino-5-cyclopropyl-7H- pyrrolo[2,3-d]pyrimidin-7- yl)hexahydro-3aH- cyclopenta[b]furan-3,3a-diol | 553/555 (M + 1/ M + 3) |
| 53 | 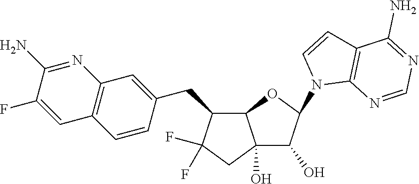 [SMILES: Nc1nc2cc(C[C@H]3[C@H]4O[C@@H](n5ccc6c(N)ncnc65)[C@H](O)[C@@]4(O)CC3(F)F)ccc2cc1F] | (2R,3R,3aS,6S,6aR)-6-[(2-amino-3- fluoroquinolin-7-yl)methyl]-2-(4- amino-7H-pyrrolo[2,3-d]pyrimidin- 7-yl)-5,5-difluorohexahydro-3aH- cyclopenta[b]furan-3,3a-diol | 487 (M + 1) |
| 54 | 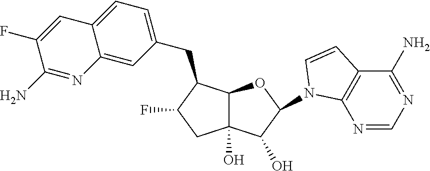 [SMILES: Nc1nc2cc(C[C@H]3[C@H]4O[C@@H](n5ccc6c(N)ncnc65)[C@H](O)[C@@]4(O)C[C@@H]3F)ccc2cc1F] | (2R,3R,3aS,5S,6S,6aR)-6-[(2- amino-3-fluoroquinolin-7- yl)methyl]-2-(4-amino-7H- pyrrolo[2,3-d]pyrimidin-7-yl)-5- fluorohexahydro-3aH- cyclopenta[b]furan-3,3a-diol | 469 (M + 1) |
| 55 | 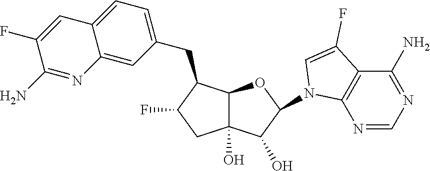 [SMILES: Nc1nc2cc(C[C@H]3[C@H]4O[C@@H](n5cc(F)c6c(N)ncnc65)[C@H](O)[C@@]4(O)C[C@@H]3F)ccc2cc1F] | (2R,3R,3aS,5S,6S,6aR)-2-(4- amino-5-fluoro-7H-pyrrolo[2,3- d]pyrimidin-7-yl)-6-[(2-amino-3- fluoroquinolin-7-yl)methyl]-5- fluorohexahydro-3aH- cyclopenta[b]furan-3,3a-diol, 2,2,2-trifluoroethanol | 487 (M + 1) |
| 56 | 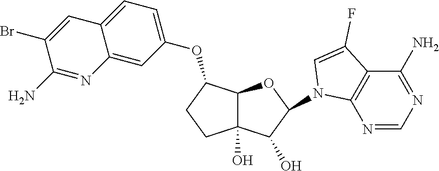 [SMILES: Nc1nc2cc(O[C@H]3CC[C@@]4(O)[C@@H]3O[C@@H](n3cc(F)c5c(N)ncnc53)[C@@H]4O)ccc2cc1Br] | (2R,3R,3aS,6S,6aR)-6-((2-amino- 3-bromoquinolin-7-yl)oxy)-2-(4- amino-5-fluoro-7H-pyrrolo[2,3- d]pyrimidin-7-yl)hexahydro-3aH- cyclopenta[b]furan-3,3a-diol | 531/533 (M + 1/ M + 3) |
| 57 | 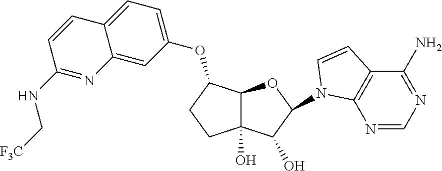 [SMILES: Nc1ncnc2c1ccn2[C@@H]1O[C@@H]2[C@@H](Oc3ccc4ccc(NCC(F)(F)F)nc4c3)CC[C@]2(O)[C@H]1O] | (2R,3R,3aS,6S,6aR)-2-(4-amino- 7H-pyrrolo[2,3-d]pyrimidin-7-yl)- 6-((2-((2,2,2- trifluoroethyl)amino)quinolin-7- yl)oxy)hexahydro-3aH- cyclopenta[b]furan-3,3a-diol | 517 (M + 1) |
| 58 | 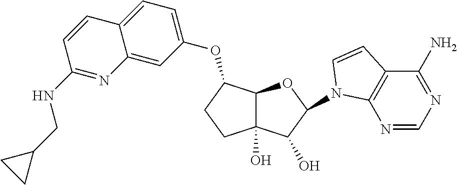 [SMILES: Nc1ncnc2c1ccn2[C@@H]1O[C@@H]2[C@@H](Oc3ccc4ccc(NCC5CC5)nc4c3)CC[C@]2(O)[C@H]1O] | (2R,3R,3aS,6S,6aR)-2-(4-amino- 7H-pyrrolo[2,3-d]pyrimidin-7-yl)- 6-((2- ((cyclopropylmethyl)amino) quinolin-7-yl)oxy)hexahydro-3aH- cyclopenta[b]furan-3,3a-diol | 489 (M + 1) |
| 59 | 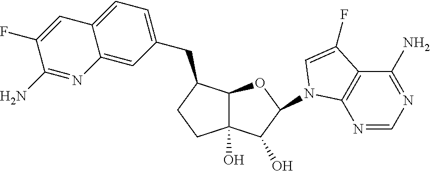 [SMILES: Nc1nc2cc(C[C@@H]3CC[C@@]4(O)[C@@H]3O[C@@H](n3cc(F)c5c(N)ncnc53)[C@@H]4O)ccc2cc1F] | (2R,3R,3aS,6S,6aR)-6-((2-amino- 3-fluoroquinolin-7-yl)methyl)-2-(4- amino-5-fluoro-7H-pyrrolo[2,3- d]pyrimidin-7-yl)hexahydro-3aH- cyclopenta[b]furan-3,3a-diol | 469 (M + 1) |
| 60 | 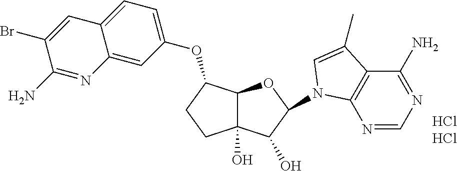 [SMILES: Cc1cn([C@@H]2O[C@@H]3[C@@H](Oc4ccc5cc(Br)c(N)nc5c4)CC[C@]3(O)[C@H]2O)c2ncnc(N)c12 ; Cl ; Cl] | (2R,3R,3aS,6S,6aR)-6-((2-amino- 3-bromoquinolin-7-yl)oxy)-2-(4- amino-5-methyl-7H-pyrrolo[2,3- d]pyrimidin-7-yl)hexahydro-3aH- cyclopenta[b]furan-3,3a-diol | 527/529 (M + 1/ M + 3) |
| 61 | 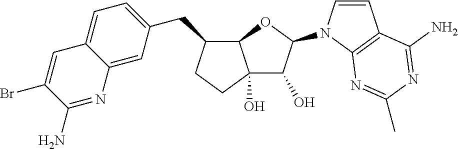 [SMILES: Cc1nc(N)c2ccn([C@@H]3O[C@@H]4[C@H](Cc5ccc6cc(Br)c(N)nc6c5)CC[C@]4(O)[C@H]3O)c2n1] | (2R,3R,3aS,6S,6aR)-2-(4-amino-2- methyl-7H-pyrrolo[2,3- d]pyrimidin-7-yl)-6-((2-amino-3- bromoquinolin-7- yl)methyl)hexahydro-3aH- cyclopenta[b]furan-3,3a-diol | 525/527 (M + 1/ M + 3) |
| 62 | 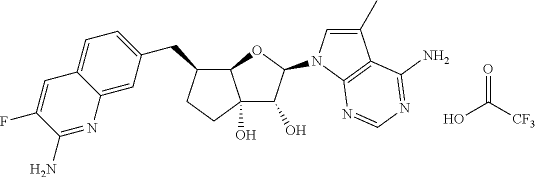 [SMILES: Cc1cn([C@@H]2O[C@@H]3[C@H](Cc4ccc5cc(F)c(N)nc5c4)CC[C@]3(O)[C@H]2O)c2ncnc(N)c12 ; O=C(O)C(F)(F)F] | (2R,3R,3aS,6S,6aR)-6-((2-amino- 3-fluoroquinolin-7-yl)methyl)-2-(4- amino-5-methyl-7H-pyrrolo[2,3- d]pyrimidin-7-yl)hexahydro-3aH- cyclopenta[b]furan-3,3a-diol 2,2,2- trifluoroacetate | 465 (M + 1) |
| 63 | 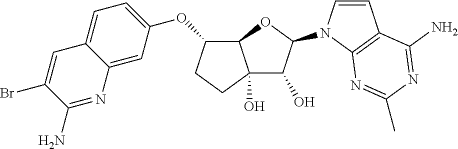 [SMILES: Cc1nc(N)c2ccn([C@@H]3O[C@@H]4[C@@H](Oc5ccc6cc(Br)c(N)nc6c5)CC[C@]4(O)[C@H]3O)c2n1] | (2R,3R,3aS,6S,6aR)-2-(4-amino- 2-methyl-7H-pyrrolo[2,3- d]pyrimidin-7-yl)-6-((2-amino-3- bromoquinolin-7- yl)oxy)hexahydro-3aH- cyclopenta[b]furan-3,3a-diol | 527/529 (M + 1/ M + 3) |
| 64 | 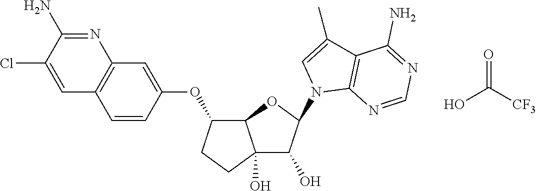 [SMILES: Cc1cn([C@@H]2O[C@@H]3[C@@H](Oc4ccc5cc(Cl)c(N)nc5c4)CC[C@]3(O)[C@H]2O)c2ncnc(N)c12 ; O=C(O)C(F)(F)F] | (2R,3R,3aS,6S,6aR)-6-((2-amino- 3-chloroquinolin-7-yl)oxy)-2-(4- amino-5-methyl-7H-pyrrolo[2,3- d]pyrimidin-7-yl)hexahydro-3aH- cyclopenta[b]furan-3,3a-diol 2,2,2- trifluoroacetate | 483 (M + 1) |
| 65 | 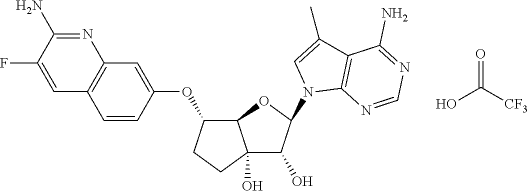 [SMILES: Cc1cn([C@@H]2O[C@@H]3[C@@H](Oc4ccc5cc(F)c(N)nc5c4)CC[C@]3(O)[C@H]2O)c2ncnc(N)c12 ; O=C(O)C(F)(F)F] | (2R,3R,3aS,6S,6aR)-6-((2-amino- 3-fluoroquinolin-7-yl)oxy)-2-(4- amino-5-methyl-7H-pyrrolo[2,3- d]pyrimidin-7-yl)hexahydro-3aH- cyclopenta[b]furan-3,3a-diol 2,2,2- trifluoroacetate | 467 (M + 1) |
| 66 | 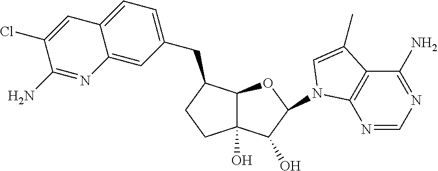 [SMILES: Cc1cn([C@@H]2O[C@@H]3[C@H](Cc4ccc5cc(Cl)c(N)nc5c4)CC[C@]3(O)[C@H]2O)c2ncnc(N)c12] | (2R,3R,3aS,6S,6aR)-6-((2-amino- 3-chloroquinolin-7-yl)methyl)-2-(4- amino-5-methyl-7H-pyrrolo[2,3- d]pyrimidin-7-yl)hexahydro-3aH- cyclopenta[b]furan-3,3a-diol | 481 (M + 1) |

Examples 67-70: Examples 67-70 in Table 17 were synthesized by applying the Mitsunobu protocol described in step 3 of the synthesis of Example 45 followed by application of the ammnolysis protocol described in step 11 of Example 10 & 11. Di-tert-butyl (2-fluoro-7H-pyrrolo[2,3-d]pyrimidin-4-yl)imidodicarbonate and (3R,3aS,6S,6aR)-6-((2-amino-3-fluoroquinolin-7-yl)methyl)hexahydro-3aH-cyclopenta[b]furan-2,3,3a-triol in the Mitsunobu sequence were substituted with an appropriate nucleobase and triol respectively.

TABLE 17

| Ex | Structure | Name | MS |
|---|---|---|---|
| 67 | 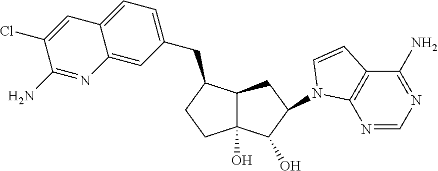 [SMILES: Nc1nc2cc(C[C@@H]3CC[C@@]4(O)[C@@H]3C[C@@H](n3ccc5c(N)ncnc53)[C@@H]4O)ccc2cc1Cl] | (2R,3R,3aS,6S,6aR)-6-((2- amino-3-chloroquinolin-7- yl)methyl)-2-(4-amino-7H- pyrrolo[2,3-d]pyrimidin-7- yl)hexahydro-3aH- cyclopenta[b]furan-3,3a-diol | 467 (M + 1) |
| 68 | 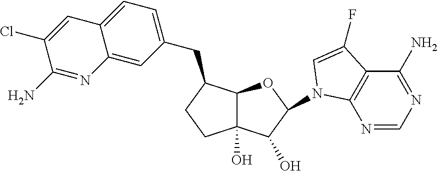 [SMILES: Nc1nc2cc(C[C@@H]3CC[C@@]4(O)[C@@H]3O[C@@H](n3cc(F)c5c(N)ncnc53)[C@@H]4O)ccc2cc1Cl] | (2R,3R,3aS,6S,6aR)-6-((2- amino-3-chloroquinolin-7- yl)methyl)-2-(4-amino-5- fluoro-7H-pyrrolo[2,3- d]pyrimidin-7-yl)hexahydro- 3aH-cyclopenta[b]furan-3,3a- diol | 485 (M + 1) |
| 69 | 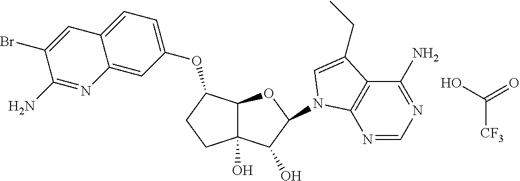 [SMILES: CCc1cn([C@@H]2O[C@@H]3[C@@H](Oc4ccc5cc(Br)c(N)nc5c4)CC[C@]3(O)[C@H]2O)c2ncnc(N)c12 ; O=C(O)C(F)(F)F] | (2R,3R,3aS,6S,6aR)-6-((2- amino-3-bromoquinolin-7- yl)oxy)-2-(4-amino-5-ethyl- 7H-pyrrolo[2,3-d]pyrimidin-7- yl)hexahydro-3aH- cyclopenta[b]furan-3,3a-diol | 541/543 (M + 1/ M + 3) |
| 70 | 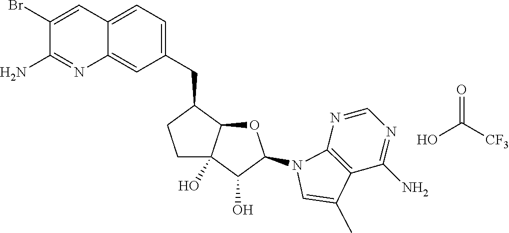 | (2R,3R,3aS,6S,6aR)-6-((2- amino-3-bromoquinolin-7- yl)methyl)-2-(4-amino-5- methyl-7H-pyrrolo[2,3- d]pyrimidin-7-yl)hexahydro- 3aH-cyclopenta[b]furan-3,3a- diol 2,2,2-trifluoroacetate | 525/527 (M + 1/ M + 3) |

Examples 71-75: Examples 71-75 in Table 18 were synthesized by applying the Mitsunobu protocol described in step 3 of the synthesis of Example 45 followed by application of the ammnolysis protocol described in step 12 of intermediate 15. Di-tert-butyl (2-fluoro-7H-pyrrolo[2,3-d]pyrimidin-4-yl)imidodicarbonate and (3R,3aS,6S,6aR)-6-((2-amino-3-fluoroquinolin-7-yl)methyl)hexahydro-3aH-cyclopenta[b]furan-2,3,3a-triol in the Mitsunobu sequence were substituted with an appropriate nucleobase and triol respectively.

TABLE 18

| Ex | Structure | Name | MS |
|---|---|---|---|
| 71 | 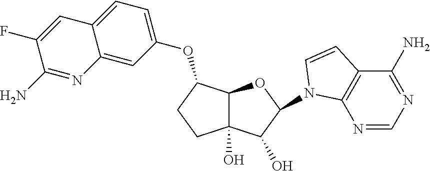 [SMILES: Nc1nc2cc(O[C@H]3CC[C@@]4(O)[C@@H]3O[C@@H](n3ccc5c(N)ncnc53)[C@@H]4O)ccc2cc1F] | (2R,3R,3aS,6S,6aR)-6-((2-amino- 3-fluoroquinolin-7-yl)oxy)-2-(4- amino-7H-pyrrolo[2,3-d]pyrimidin- 7-yl)hexahydro-3aH- cyclopenta[b]furan-3,3a-diol | 453 (M + 1) |
| 72 | 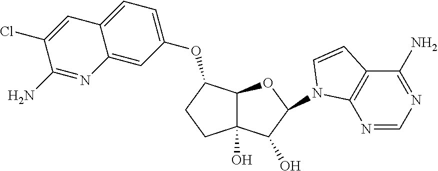 [SMILES: Nc1nc2cc(O[C@H]3CC[C@@]4(O)[C@@H]3O[C@@H](n3ccc5c(N)ncnc53)[C@@H]4O)ccc2cc1Cl] | (2R,3R,3aS,6S,6aR)-6-((2-amino- 3-chloroquinolin-7-yl)oxy)-2-(4- amino-7H-pyrrolo[2,3-d]pyrimidin- 7-yl)hexahydro-3aH- cyclopenta[b]furan-3,3a-diol | 469 (M + 1) |
| 73 | 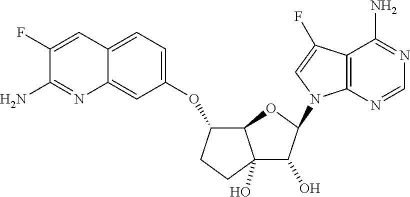 [SMILES: Nc1nc2cc(O[C@H]3CC[C@@]4(O)[C@@H]3O[C@@H](n3cc(F)c5c(N)ncnc53)[C@@H]4O)ccc2cc1F] | (2R,3R,3aS,6S,6aR)-6-((2-amino- 3-fluoroquinolin-7-yl)oxy)-2-(4- amino-5-fluoro-7H-pyrrolo[2,3- d]pyrimidin-7-yl)hexahydro-3aH- cyclopenta[b]furan-3,3a-diol | 471 (M + 1) |
| 74 | 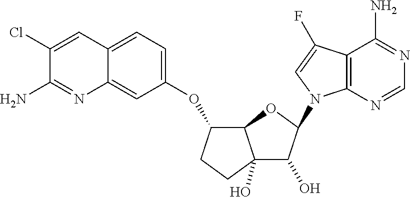 [SMILES: Nc1nc2cc(O[C@H]3CC[C@@]4(O)[C@@H]3O[C@@H](n3cc(F)c5c(N)ncnc53)[C@@H]4O)ccc2cc1Cl] | (2R,3R,3aS,6S,6aR)-6-((2-amino- 3-chloroquinolin-7-yl)oxy)-2-(4- amino-5-fluoro-7H-pyrrolo[2,3- d]pyrimidin-7-yl)hexahydro-3aH- cyclopenta[b]furan-3,3a-diol | 487 (M + 1) |
| 75 | 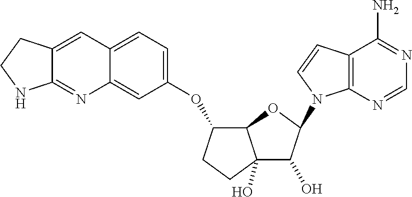 [SMILES: Nc1ncnc2c1ccn2[C@@H]1O[C@@H]2[C@@H](Oc3ccc4cc5c(nc4c3)NCC5)CC[C@]2(O)[C@H]1O] | (2R,3R,3aS,6S,6aR)-2-(4-amino- 7H-pyrrolo[2,3-d]pyrimidin-7-yl)- 6-((2,3-dihydro-1H-pyrrolo[2,3- b]quinolin-7-yl)oxy)hexahydro- 3aH-cyclopenta[b]furan-3,3a-diol | 461 (M + 1) |

Example 76: Example 76 in Table 19 was synthesized by applying the Mitsunobu protocol described in step 7 of the synthesis of Example 14 followed by application of the aminolysis protocol described in step 12 of intermediate 15. 4-chloro-7H-pyrrolo[2,3-d]pyrimidine and (3R,4S,5R)-5-(2-(3-bromo-2-((4-methoxybenzyl)amino)quinolin-7-yl)ethyl)-4-methyltetrahydrofuran-2,3,4-triol in the Mitsunobu sequence were substituted with an appropriate nucleobase and triol respectively.

TABLE 19

| Ex | Structure | Name | MS |
|---|---|---|---|
| 76 | 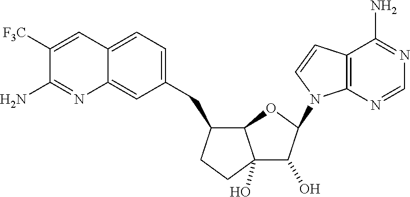 [SMILES: Nc1nc2cc(C[C@@H]3CC[C@@]4(O)[C@@H]3O[C@@H](n3ccc5c(N)ncnc53)[C@@H]4O)ccc2cc1C(F)(F)F] | (2R,3R,3aS,6S,6aR)-6-((2- amino-3- (trifluoromethyl)quinolin-7- yl)methyl)-2-(4-amino-7H- pyrrolo[2,3-d]pyrimidin-7- yl)hexahydro-3aH- cyclopenta[b]furan-3,3a-diol | 501 (M+) |

Examples 77-84: Examples 77-84 in Table 20 were synthesized by applying the Mitsunobu protocol described in step 3 of the synthesis of Example 45. Di-tert-butyl (2-fluoro-7H-pyrrolo[2,3-d]pyrimidin-4-yl)imidodicarbonate and (3R,3aS,6S,6aR)-6-((2-amino-3-fluoroquinolin-7-yl)methyl)hexahydro-3aH-cyclopenta[b]furan-2,3,3a-triol in the Mitsunobu sequence were substituted with an appropriate nucleobase and triol respectively.

TABLE 20

| Ex | Structure | Name | MS |
|---|---|---|---|
| 77 | 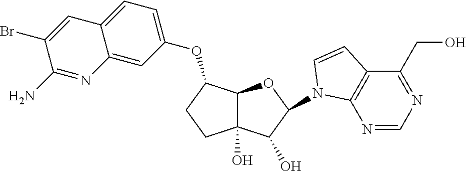 [SMILES: Nc1nc2cc(O[C@H]3CC[C@@]4(O)[C@@H]3O[C@@H](n3ccc5c(CO)ncnc53)[C@@H]4O)ccc2cc1Br] | (2R,3R,3aS,6S,6aR)-6-[(2- amino-3-bromoquinolin-7- yl)oxy]-2-[4- (hydroxymethyl)-7H- pyrrolo[2,3-d]pyrimidin-7- yl]hexahydro-3aH- cyclopenta[b]furan-3,3a- diol | 528/530 (M + 1/M + 3) |
| 78 | 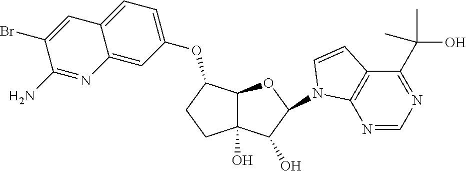 [SMILES: CC(C)(O)c1ncnc2c1ccn2[C@@H]1O[C@@H]2[C@@H](Oc3ccc4cc(Br)c(N)nc4c3)CC[C@]2(O)[C@H]1O] | (2R,3R,3aS,6S,6aR)-6-[(2- amino-3-bromoquinolin-7- yl)oxy]-2-[4-(2- hydroxypropan-2-yl)-7H- pyrrolo[2,3-d]pyrimidin-7- yl]hexahydro-3aH- cyclopenta[b]furan-3,3a- diol | 556/558 (M + 1/M + 3) |
| 79 | 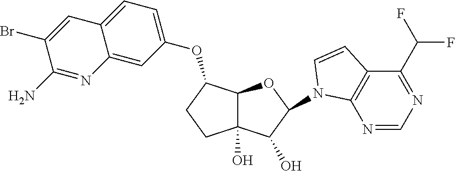 [SMILES: Nc1nc2cc(O[C@H]3CC[C@@]4(O)[C@@H]3O[C@@H](n3ccc5c(C(F)F)ncnc53)[C@@H]4O)ccc2cc1Br] | (2R,3R,3aS,6S,6aR)-6-((2- amino-3-bromoquinolin-7- yl)oxy)-2-(4- (difluoromethyl)-7H- pyrrolo[2,3-d]pyrimidin-7- yl)hexahydro-3aH- cyclopenta[b]furan-3,3a- diol | 548/550 (M + 1/M + 3) |
| 80 | 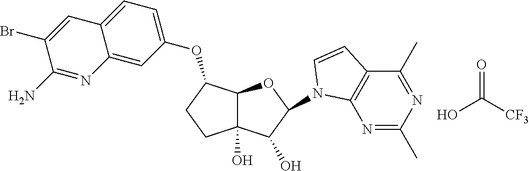 | (2R,3R,3aS,6S,6aR)-6-((2- amino-3-bromoquinolin-7- yl)oxy)-2-(2,4-dimethyl- 7H-pyrrolo[2,3- d]pyrimidin-7- yl)hexahydro-3aH- cyclopenta[b]furan-3,3a- diol 2,2,2-trifluoroacetate | 526/528 (M + 1/M + 3) |
| 81 | 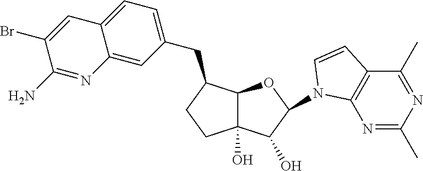 [SMILES: Cc1nc(C)c2ccn([C@@H]3O[C@@H]4[C@H](Cc5ccc6cc(Br)c(N)nc6c5)CC[C@]4(O)[C@H]3O)c2n1] | (2R,3R,3aS,6S,6aR)-6-((2- amino-3-bromoquinolin-7- yl)methyl)-2-(2,4-dimethyl- 7H-pyrrolo[2,3- d]pyrimidin-7- yl)hexahydro-3aH- cyclopenta[b]furan-3,3a- diol | 524/526 (M + 1/M + 3) |
| 82 | 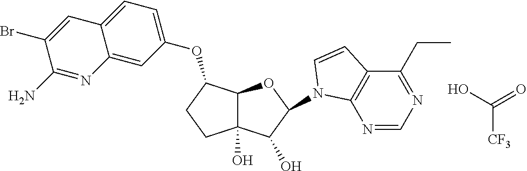 [SMILES: CCc1ncnc2c1ccn2[C@@H]1O[C@@H]2[C@@H](Oc3ccc4cc(Br)c(N)nc4c3)CC[C@]2(O)[C@H]1O ; O=C(O)C(F)(F)F] | (2R,3R,3aS,6S,6aR)-6-((2- amino-3-bromoquinolin-7- yl)oxy)-2-(4-amino-5- ethyl-7H-pyrrolo[2,3- d]pyrimidin-7- yl)hexahydro-3aH- cyclopenta[b]furan-3,3a- diol 2,2,2-trifluoroacetate | 526/528 (M + 1/M + 3) |
| 83 | 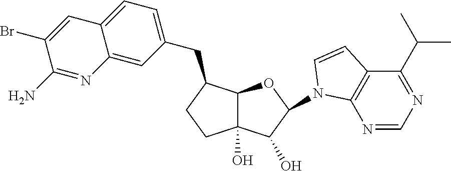 [SMILES: CC(C)c1ncnc2c1ccn2[C@@H]1O[C@@H]2[C@H](Cc3ccc4cc(Br)c(N)nc4c3)CC[C@]2(O)[C@H]1O] | (2R,3R,3aS,6S,6aR)-6-((2- amino-3-bromoquinolin-7- yl)methyl)-2-(4-isopropyl- 7H-pyrrolo[2,3- d]pyrimidin-7- yl)hexahydro-3aH- cyclopenta[b]furan-3,3a- diol | 538/540 (M + 1/M + 3) |
| 84 | 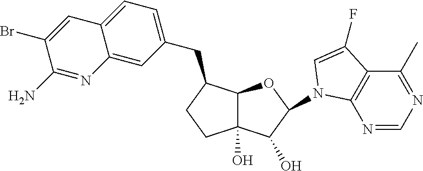 [SMILES: Cc1ncnc2c1c(F)cn2[C@@H]1O[C@@H]2[C@H](Cc3ccc4cc(Br)c(N)nc4c3)CC[C@]2(O)[C@H]1O] | (2R,3R,3aS,6S,6aR)-6-((2- amino-3-bromoquinolin-7- yl)methyl)-2-(5-fluoro-4- methyl-7H-pyrrolo[2,3- d]pyrimidin-7- yl)hexahydro-3aH- cyclopenta[b]furan-3,3a- diol | 528/530 (M + 1/M + 3) |

Examples 85-91: Examples 85-91 in Table 21 were synthesized by applying the Mitsunobu protocol described in Example 14, Step 7. 4-chloro-7H-pyrrolo[2,3-d]pyrimidine and (3R,4S,5R)-5-(2-(3-bromo-2-((4-methoxybenzyl)amino)quinolin-7-yl)ethyl)-4-c methyltetrahydrofuran-2,3,4-triol in the Mitsunobu sequence were substituted with an appropriate nucleobase and triol respectively.

TABLE 21

| Ex | Structure | Name | MS |
|---|---|---|---|
| 85 | 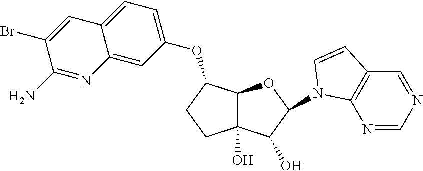 [SMILES: Nc1nc2cc(O[C@H]3CC[C@@]4(O)[C@@H]3O[C@@H](n3ccc5cncnc53)[C@@H]4O)ccc2cc1Br] | (2R,3R,3aS,6S,6aR)-6-((2- amino-3-bromoquinolin-7- yl)oxy)-2-(7H-pyrrolo[2,3- d]pyrimidin-7-yl)hexahydro-3aH- cyclopenta[b]furan-3,3a-diol | 498/500 (M + 1/M + 3) |
| 86 | 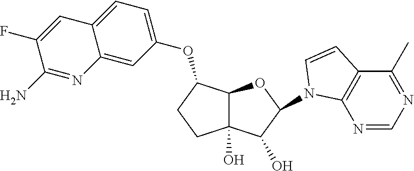 [SMILES: Cc1ncnc2c1ccn2[C@@H]1O[C@@H]2[C@@H](Oc3ccc4cc(F)c(N)nc4c3)CC[C@]2(O)[C@H]1O] | (2R,3R,3aS,6S,6aR)-6-((2- amino-3-fluoroquinolin-7- yl)oxy)-2-(4-methyl-7H- pyrrolo[2,3-d]pyrimidin-7- yl)hexahydro-3aH- cyclopenta[b]furan-3,3a-diol | 452 (M + 1) |
| 87 | 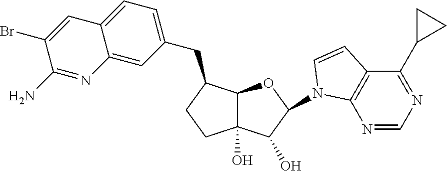 [SMILES: Nc1nc2cc(C[C@@H]3CC[C@@]4(O)[C@@H]3O[C@@H](n3ccc5c(C6CC6)ncnc53)[C@@H]4O)ccc2cc1Br] | (2R,3R,3aS,6S,6aR)-6-((2- amino-3-bromoquinolin-7- yl)methyl)-2-(4-cyclopropyl-7H- pyrrolo[2,3-d]pyrimidin-7- yl)hexahydro-3aH- cyclopenta[b]furan-3,3a-diol | 536/538 (M + 1/M + 3) |
| 88 | 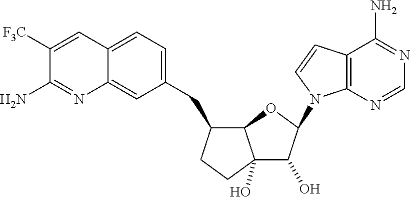 [SMILES: Nc1nc2cc(C[C@@H]3CC[C@@]4(O)[C@@H]3O[C@@H](n3ccc5c(N)ncnc53)[C@@H]4O)ccc2cc1C(F)(F)F] | (2R,3R,3aS,6S,6aR)-6-((2- amino-3- (trifluoromethyl)quinolin-7- yl)methyl)-2-(4-methyl-7H- pyrrolo[2,3-d]pyrimidin-7- yl)hexahydro-3aH- cyclopenta[b]furan-3,3a-diol | 500 (M + 1) |
| 89 | 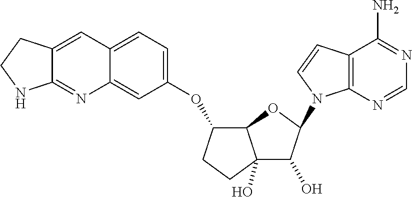 [SMILES: Nc1ncnc2c1ccn2[C@@H]1O[C@@H]2[C@@H](Oc3ccc4cc5c(nc4c3)NCC5)CC[C@]2(O)[C@H]1O] | (2R,3R,3aS,6S,6aR)-6-((2,3- dihydro-1H-pyrrolo[2,3- b]quinolin-7-yl)oxy)-2-(4- methyl-7H-pyrrolo[2,3- d]pyrimidin-7-yl)hexahydro-3aH- cyclopenta[b]furan-3,3a-diol | 460 (M + 1) |
| 90 | 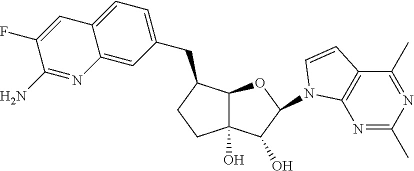 [SMILES: Cc1nc(C)c2ccn([C@@H]3O[C@@H]4[C@H](Cc5ccc6cc(F)c(N)nc6c5)CC[C@]4(O)[C@H]3O)c2n1] | (2R,3R,3aS,6S,6aR)-6-((2- amino-3-fluoroquinolin-7- yl)methyl)-2-(2,4-dimethyl-7H- pyrrolo[2,3-d]pyrimidin-7- yl)hexahydro-3aH- cyclopenta[b]furan-3,3a-diol | 464 (M + 1) |
| 91 | 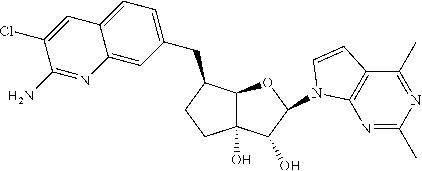 [SMILES: Cc1nc(C)c2ccn([C@@H]3O[C@@H]4[C@H](Cc5ccc6cc(Cl)c(N)nc6c5)CC[C@]4(O)[C@H]3O)c2n1] | (2R,3R,3aS,6S,6aR)-6-((2- amino-3-chloroquinolin-7- yl)methyl)-2-(2,4-dimethyl-7H- pyrrolo[2,3-d]pyrimidin-7- yl)hexahydro-3aH- cyclopenta[b]furan-3,3a-diol | 480 (M + 1) |

### Example 92

### (2R,3R,3aS,6S,6aR)-6-((2-amino-3-bromoquinolin-7-yl)methyl)-2-(4-(methylamino)-7H-pyrrolo[2,3-d]pyrimidin-7-yl)hexahydro-3aH-cyclopenta[b]furan-3,3a-diol

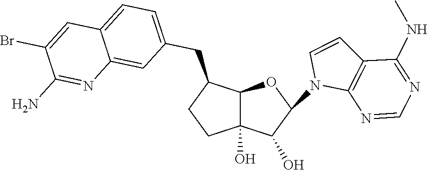 [SMILES: CNc1ncnc2c1ccn2[C@@H]1O[C@@H]2[C@H](Cc3ccc4cc(Br)c(N)nc4c3)CC[C@]2(O)[C@H]1O]

Step 1: To a vial containing (2R,3R,3aS,6S,6aR)-6-((3-bromo-2-((4-methoxybenzyl)amino)quinolin-7-yl)methyl)-2-(4-chloro-7H-pyrrolo[2,3-d]pyrimidin-7-yl)hexahydro-2H-cyclopenta[b]furan-3,3a-diol (102 mg, 0.157 mmol) dissolved in dioxane (3000 μl) was added methylamine (2.0 M in THF, 6.26 mL, 12.5 mmol) at room temperature. The mixture was sealed and heated at 70° C. for 6 h. The mixture was concentrated under reduced pressure to afford (2R,3R,3aS,6S,6aR)-6-((3-bromo-2-((4-methoxybenzyl)amino)quinolin-7-yl)methyl)-2-(4-(methylamino)-7H-pyrrolo[2,3-d]pyrimidin-7-yl)hexahydro-2H-cyclopenta[b]furan-3,3a-diol as a foam. MS: 645/647 (M+1/M+3).

Step 2: To (2R,3R,3aS,6aR)-6-((3-bromo-2-((4-methoxybenzyl)amino)quinolin-7-yl)methyl)-2-(4-(methylamino)-7H-pyrrolo[2,3-d]pyrimidin-7-yl)hexahydro-2H-cyclopenta[b]furan-3,3a-diol (101 mg, 0.156 mmol) dissolved in DCM (1304 μl) was added TFA (1300 μl, 16.87 mmol) at room temperature. The mixture was stirred for 5.5 h at 40° C. and then turned off the heat and let the reaction stir at room temperature overnight. The reaction was heated at 40° C. for 2 h. The mixture was concentrated under reduced pressure and purified by mass triggered reverse phase column chromatography (ACN:water with 0.1% NH₄OH modifier) to afford (2R,3R,3aS,6S,6aR)-6-((2-amino-3-bromoquinolin-7-yl)methyl)-2-(4-(methylamino)-7H-pyrrolo[2,3-d]pyrimidin-7-yl)hexahydro-2H-cyclopenta[b]furan-3,3a-diol as a solid. MS: 525/527 (M+1/M+3). 1H NMR (600 MHz, DMSO-d6) δ 8.31 (s, 1H), 8.19 (s, 1H), 7.55 (d, J=8.2 Hz, 2H), 7.45 (d, J=3.6 Hz, 1H), 7.28 (s, 1H), 7.09 (dd, J=8.2, 1.4 Hz, 1H), 6.67 (d, J=3.6 Hz, 1H), 6.57 (s, 2H), 5.90 (d, J=8.1 Hz, 1H), 5.25 (d, J=7.1 Hz, 1H), 5.07 (s, 1H), 4.15 (t, J=7.4 Hz, 1H), 3.96 (d, J=5.7 Hz, 1H), 2.99 (d, J=4.5 Hz, 3H), 2.86-2.79 (m, 1H), 2.66-2.58 (m, 1H), 2.29-2.22 (m, 1H), 1.98-1.91 (m, 1H), 1.78-1.65 (m, 2H), 1.58-1.49 (in, 1H).

Examples 93-97: Examples 93-97 in Table 22 were synthesized in an analogously to Example 92, Steps 1-2 of Example 92 by substituting the (2R,3R,3aS,6S,6aR)-6-((3-bromo-2-((4-methoxybenzyl)amino)quinolin-7-yl)methyl)-2-(4-chloro-7H-pyrrolo[2,3-d]pyrimidin-7-yl)hexahydro-2H-cyclopenta[b]furan-3,3a-diol with an appropriate chloro nucleobase.

TABLE 22

| Ex | Structure | Name | MS |
|---|---|---|---|
| 93 | 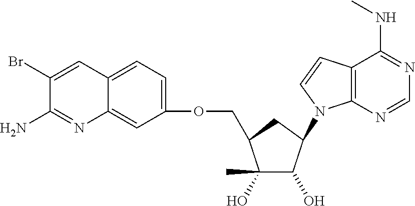 [SMILES: CNc1ncnc2c1ccn2[C@@H]1C[C@H](COc2ccc3cc(Br)c(N)nc3c2)[C@@](C)(O)[C@H]1O] | (1R,2S,3R,5R)-5-(((2-amino-3- bromoquinolin-7-yl)oxy)methyl)- 1-methyl-3-(4-(methylamino)- 7H-pyrrolo[2,3-d]pyrimidin-7- yl)cyclopentane-1,2-diol | 513/515 (M + 1/M + 3) |
| 94 | 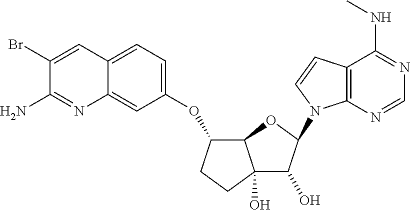 [SMILES: CNc1ncnc2c1ccn2[C@@H]1O[C@@H]2[C@@H](Oc3ccc4cc(Br)c(N)nc4c3)CC[C@]2(O)[C@H]1O] | (2R,3R,3aS,6S,6aR)-6-((2- amino-3-bromoquinolin-7- yl)oxy)-2-(4-(methylamino)-7H- pyrrolo[2,3-d]pyrimidin-7- yl)hexahydro-3aH- cyclopenta[b]furan-3,3a-diol | 527/529 (M + 1/M + 3) |
| 95 | 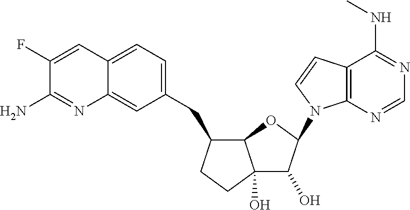 [SMILES: CNc1ncnc2c1ccn2[C@@H]1O[C@@H]2[C@H](Cc3ccc4cc(F)c(N)nc4c3)CC[C@]2(O)[C@H]1O] | (2R,3R,3aS,6S,6aR)-6-((2- amino-3-fluoroquinolin-7- yl)methyl)-2-(4-(methylamino)- 7H-pyrrolo[2,3-d]pyrimidin-7- yl)hexahydro-3aH- cyclopenta[b]furan-3,3a-diol | 465 (M + 1) |
| 96 | 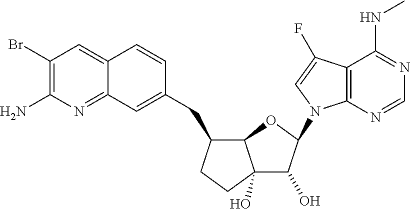 [SMILES: CNc1ncnc2c1c(F)cn2[C@@H]1O[C@@H]2[C@H](Cc3ccc4cc(Br)c(N)nc4c3)CC[C@]2(O)[C@H]1O] | (2R,3R,3aS,6S,6aR)-6-((2- amino-3-bromoquinolin-7- yl)methyl)-2-(5-fluoro-4- (methylamino)-7H-pyrrolo[2,3- d]pyrimidin-7-yl)hexahydro- 3aH-cyclopenta[b]furan-3,3a-diol | 543/545 (M + 1/M + 3) |
| 97 | 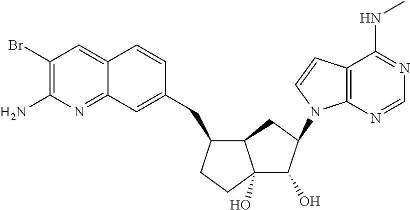 [SMILES: CNc1ncnc2c1ccn2[C@@H]1C[C@@H]2[C@H](Cc3ccc4cc(Br)c(N)nc4c3)CC[C@]2(O)[C@H]1O] | (1S,2R,3aR,4S,6aR)-4-((2- amino-3-bromoquinolin-7- yl)methyl)-2-(4-(methylamino)- 7H-pyrrolo[2,3-d]pyrimidin-7- yl)hexahydropentalene-1,6a(1H)- diol | 523/525 (M + 1/M + 3) |

### Example 98

### (2R,3S,4R,5R)-5-(4-amino-7H-pyrrolo[2,3-d]pyrimidin-7-yl)-2-methyl-2-((quinolin-7-yloxy)methyl)tetrahydrofuran-3,4-diol

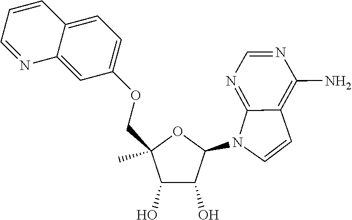 [SMILES: C[C@]1(COc2ccc3cccnc3c2)O[C@@H](n2ccc3c(N)ncnc32)[C@H](O)[C@@H]1O]

Step 1: To a solution of ((3aS,4R,6R,6aR)-6-(4-chloro-7H-pyrrolo[2,3-d]pyrimidin-7-yl)-2,2,4-trimethyltetrahydrofuro[3,4-d][1,3]dioxol-4-yl)methanol (400 mg, 1.18 mmol), quinolin-7-ol (205 mg, 1.41 mmol) and triphenylphosphine (1578 mg, 3.06 mmol) were stirred in anhydrous THF (12 mL) under nitrogen gas. DIAD (0.572 mL, 2.94 mmol) was added dropwise at 0° C. The mixture was stirred at room temperature overnight. The reaction mixture was filtered and washed with MeOH. The filtrate was concentrated under reduced pressure and purified by column chromatography on silica (EtOAc/PE 0-50% and then MeOH/DCM 0-5%) to afford 7-(((3aS,4R,6R,6aR)-6-(4-chloro-7H-pyrrolo[2,3-d]pyrimidin-7-yl)-2,2,4-trimethyltetrahydrofuro[3,4-d][1,3]dioxol-4-yl)methoxy)quinoline as a solid. Then ammonia (28% in water) (1 mL, 12.94 mmol) was added to a stirred solution of 7-(((3aS,4R,6R,6aR)-6-(4-chloro-7H-pyrrolo[2,3-d]pyrimidin-7-yl)-2,2,4-trimethyltetrahydrofuro[3,4-d][1,3]dioxol-4-yl)methoxy)quinoline (100 mg, 0.214 mmol) in dioxane (1 mL), and the mixture was stirred at 120° C. for 8.5 h. The reaction mixture was evaporated under reduced pressure and purified by reverse phase HPLC (ACN/Water) to afford 7-((3aR,4R,6R,6aS)-2,2,6-trimethyl-6-((quinolin-7-yloxy)methyl)tetrahydrofuro[3,4-d][1,3]dioxol-4-yl)-7H-pyrrolo[2,3-d]pyrimidin-4-amine as a solid. MS: 448 (M+1).

Step 2: To compound 7-((3aR,4R,6R,6aS)-2,2,6-trimethyl-6-((quinolin-7-yloxy)methyl)tetrahydrofuro[3,4-d][1,3]dioxol-4-yl)-7H-pyrrolo[2,3-d]pyrimidin-4-amine (11 mg, 0.025 mmol) was added premixed TFA (70 μl, 0.909 mmol)/water (150 μl) at 0° C. The resulting suspension was stirred at room temperature for 30 minutes. The reaction mixture was concentrated under reduced pressure. The residue was purified by reverse phase HPLC (MeCN/Water) to afford (2R,3S,4R,5R)-5-(4-amino-7H-pyrrolo[2,3-d]pyrimidin-7-yl)-2-methyl-2-((quinolin-7-yloxy)methyl)tetrahydrofuran-3,4-diol as a solid. MS: 408 (M+1). ¹H NMR (400 MHz, Methanol-d4) δ 8.79 (dd, J=4.5, 1.7 Hz, 1H), 8.32 (d, J=6.7 Hz, 1H), 8.10 (s, 1H), 7.91 (d, J=9.0 Hz, 1H), 7.48-7.37 (m, 5H), 6.62 (d, J=3.7 Hz, 1H), 6.30 (d, J=6.7 Hz, 1H), 4.45 (d, J=5.5 Hz, 1H), 4.32-4.21 (m, 2H), 1.53 (s, 3H).

Example 99: Example 99 in Table 23 was synthesized by following Steps 1-2 of Example 98 above starting from Intermediate 28.

TABLE 23

| Ex | Structure | Name | MS |
|---|---|---|---|
| 99 | 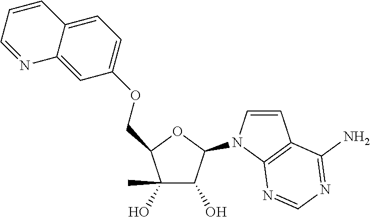 [SMILES: C[C@@]1(O)[C@@H](COc2ccc3cccnc3c2)O[C@@H](n2ccc3c(N)ncnc32)[C@@H]1O] | (2R,3S,4R,5R)-5-(4-amino-7H- pyrrolo[2,3-d]pyrimidin-7-yl)-3- methyl-2-((quinolin-7- yloxy)methyl)tetrahydrofuran-3,4- diol | 408 (M + 1) |

### Example 100

### (1S,2R,3R,5R)-3-(((2-amino-3-bromoquinolin-7-yl)oxy)methyl)-5-(4-amino-7H-pyrrolo[2,3-d]pyrimidin-7-yl)-3-methylcyclopentane-1,2-diol

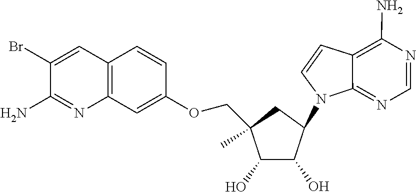

Step 1: To a solution of lithium bis(trimethylsilyl)amide (73.9 mL, 73.9 mmol) at −70° C. was added a solution of (1S,4R)-methyl 4-((tert-butoxycarbonyl)amino)cyclopent-2-enecarboxylate (8 g, 33.2 mmol) in THF (8 mL) over 2 minutes at −70° C. The resulted solution was stirred at −70° C. for 30 minutes before iodomethane (3.67 mL, 59.0 mmol) was added for 5 minutes. The reaction was warmed to −25° C. This temperature was maintained for 2 h. The resulting mixture was diluted with saturated aqueous NaHCO₃ (200 mL) and extracted with EtOAc (3×200 mL). The combined organic layers were washed with brine (200 mL), dried over anhydrous sodium sulfate, and filtered. The filtrate was concentrated under reduced pressure, and the residue was purified by column chromatography on silica (16.6% EtOAc/PE) to afford (1S,4R)-methyl 4-((tert-butoxycarbonyl)amino)-1-methylcyclopent-2-enecarboxylate as an oil. MS: 199 (M−56).

Step 2: To a stirred mixture of (1S,4R)-methyl 4-((tert-butoxycarbonyl)amino)-1-methylcyclopent-2-enecarboxylate (7.1 g, 27.8 mmol) in THF (100 mL) was added lithium tetrahydroborate (27.8 mL, 55.6 mmol) at 0° C. under an argon atmosphere. The resulting mixture was warmed to 25° C. and stirred for 16 h. The reaction mixture was quenched by MeOH (50 mL), diluted with saturated aqueous NH₄Cl (300 mL) and then extracted with EtOAc (3×200 mL). The combined organic layers were washed with brine (200 mL), dried over anhydrous sodium sulfate, and filtered. The filtrate was concentrated under reduced pressure and recrystallized from PE (100 mL). The solid was collected by filtration, and dried under reduced pressure to afford tert-butyl ((1R,4S)-4-(hydroxymethyl)-4-methylcyclopent-2-en-1-yl)carbamate as a solid. MS: 228 (M+1).

Step 3: A mixture of tert-butyl ((1R,4S)-4-(hydroxymethyl)-4-methylcyclopent-2-en-1-yl)carbamate (7.84 g, 34.5 mmol) and HCl (4 M in dioxane, 80 mL) was stirred at 25° C. for 2 h. The solution was then concentrated under reduced pressure, and the residue was triturated with ether (200 mL). The solid was collected, washed with ether (100 mL), and dried to afford ((1S,4R)-4-amino-1-methylcyclopent-2-en-1-yl)methanol hydrochloride as a solid. MS: 128 (M+1).

Step 4: To a stirred mixture of ((1S,4R)-4-amino-1-methylcyclopent-2-en-1-yl)methanol hydrochloride (5.5 g, 32.6 mmol) in 2-propanol (120 mL) were added 2-(4,6-dichloropyrimidin-5-yl)acetaldehyde (6.85 g, 35.9 mmol) and triethylamine (6.60 g, 65.2 mmol) at 25° C. under argon atmosphere. The resulting mixture was stirred at 82° C. for 16 h. The reaction mixture was quenched by water (250 mL) and extracted with ethyl acetate (3×200 mL). The combined organic layers were washed with brine (200 mL), dried over anhydrous sodium sulfate, and filtered. The filtrate was concentrated under reduced pressure, and the residue was purified by column chromatography on silica (25-32% EtOAc/PE) to afford ((1S,4R)-4-(4-chloro-7H-pyrrolo[2,3-d]pyrimidin-7-yl)-1-methylcyclopent-2-en-1-yl)methanol as a solid. MS: 264 (M+1).

Step 5: To a solution of ((1S,4R)-4-(4-chloro-7H-pyrrolo[2,3-d]pyrimidin-7-yl)-1-methylcyclopent-2-en-1-yl)methanol (300 mg, 1.138 mmol) in THF (5 mL) was added sodium phenolate (660 mg, 5.69 mmol) at 0° C. under an argon atmosphere. The reaction mixture was stirred at 66° C. for 12 h. The reaction mixture was quenched by saturated aqueous NH₄Cl (100 mL) and extracted with EtOAc (3×100 mL). The combined organic layers were washed with brine (100 mL), dried over anhydrous sodium sulfate, and filtered. The filtrate was concentrated under reduced pressure, and the residue was purified by column chromatography on silica (0-60% EtOAc/PE) to afford ((1S,4R)-1-methyl-4-(4-phenoxy-7H-pyrrolo[2,3-d]pyrimidin-7-yl)cyclopent-2-en-1-yl)methanol as a solid. MS: 322 (M+1).

Step 6: To a solution of ((1S,4R)-1-methyl-4-(4-phenoxy-7H-pyrrolo[2,3-d]pyrimidin-7-yl)cyclopent-2-en-1-yl)methanol (260 mg, 0.809 mmol) in DMF (1 mL) were added 1H-imidazole (116 mg, 1.699 mmol) and tert-butylchlorodiphenylsilane (289 mg, 1.052 mmol) in one portion at 0° C. under an argon atmosphere. The reaction mixture was stirred at 25° C. for 1.5 h. The reaction mixture was quenched by saturated aqueous NH₄Cl (150 mL) and extracted with EtOAc (3×100 mL). The combined organic layers were washed with brine (100 mL), dried over anhydrous sodium sulfate, and filtered. The filtrate was concentrated under reduced pressure, and the residue was purified by column chromatography on silica (10-20% EtOAc/PE) to afford 7-((1R,4S)-4-(((tert-butyldiphenylsilyl)oxy)methyl)-4-methylcyclopent-2-en-1-yl)-4-phenoxy-7H-pyrrolo[2,3-d]pyrimidine as a solid. MS: 560 (M+1).

Step 7: To a stirred solution of 7-((1R,4S)-4-(((tert-butyldiphenylsilyl)oxy)methyl)-4-methylcyclopent-2-en-1-yl)-4-phenoxy-7H-pyrrolo[2,3-d]pyrimidine (260 mg, 0.464 mmol) in THF (4 mL) were added pyridine (4 mL, 49.5 mmol) and osmium (VIII) oxide (1.299 mL, 0.511 mmol) at 0° C. under argon atmosphere. The resulting mixture was stirred at the same temperature for 1 h. The reaction mixture was quenched by saturated aqueous Na₂S20₃ (25 mL) and extracted with EtOAc (5×20 mL). The combined organic layers were washed with brine (50 mL), dried over anhydrous sodium sulfate, and filtered. The filtrate was concentrated under reduced pressure. The residue was dissolved in tBuOH (3.00 mL), and water (3 mL) and hydrogen sodium sulfite (5.52 mg, 0.053 mmol) were added at 25° C. under air. The resulting mixture was stirred for 20 minutes. The reaction mixture was diluted with saturated aqueous NaHSO₃ (25 mL) and extracted with EtOAc (3×25 mL). The combined organic layers were washed with brine (50 mL), dried over anhydrous sodium sulfate, and filtered. The crude residue was purified by column chromatography on silica (0-70% EtOAc/PE) to afford (3R,5R)-3-(((tert-butyldiphenylsilyl)oxy)methyl)-3-methyl-5-(4-phenoxy-7H-pyrrolo[2,3-d]pyrimidin-7-yl)cyclopentane-1,2-diol as an oil. MS: 594 (M+1).

Step 8: To a stirred solution of (3R,5R)-3-(((tert-butyldiphenylsilyl)oxy)methyl)-3-methyl-5-(4-phenoxy-7H-pyrrolo[2,3-d]pyrimidin-7-yl)cyclopentane-1,2-diol (2.2 g, 3.71 mmol) in dry acetone (12 mL) were added 4-methylbenzenesulfonic acid (0.064 g, 0.371 mmol) and 2,2-dimethoxypropane (1.93 g, 18.5 mmol). The resulting mixture was stirred at 25° C. for 3 h. EtOAc (300 mL) and saturated aqueous NaHCO₃ (100 mL) were added to the solution, the organic layer was concentrated under reduced pressure, and the residue was purified by column chromatography on silica (0-30% EtOAc/PE) to afford 7-((3aS,4R,6R,6aR)-6-(((tert-butyldiphenylsilyl)oxy)methyl)-2,2,6-trimethyltetrahydro-3aH-cyclopenta[d][1,3]dioxol-4-yl)-4-phenoxy-7H-pyrrolo[2,3-d]pyrimidine as a foam. MS: 634 (M+1).

Step 9: To a stirred solution of 7-((3aS,4R,6R,6aR)-6-(((tert-butyldiphenylsilyl)oxy)methyl)-2,2,6-trimethyltetrahydro-3aH-cyclopenta[d][1,3]dioxol-4-yl)-4-phenoxy-7H-pyrrolo[2,3-d]pyrimidine (650 mg, 1.025 mmol) in THF (5 mL) was added tetrabutylammonium fluoride (1M in THF, 3.08 mL, 3.08 mmol) at 25° C. The resulting mixture was stirred at 25° C. for 2 h. Saturated aqueous NaHCO₃ (100 mL) and EtOAc (250 mL) were added to the solution, then the organic layer was concentrated under reduced pressure, and the residue was purified by column chromatography (0-70% EtOAc/PE) to afford ((3aR,4R,6R,6aS)-2,2,4-trimethyl-6-(4-phenoxy-7H-pyrrolo[2,3-d]pyrimidin-7-yl)tetrahydro-3aH-cyclopenta[d][1,3]dioxol-4-yl)methanol as a foam. MS: 396 (M+1).

Step 10: To a stirred mixture of ((3aR,4R,6R,6aS)-2,2,4-trimethyl-6-(4-phenoxy-7H-pyrrolo[2,3-d]pyrimidin-7-yl)tetrahydro-3aH-cyclopenta[d][1,3]dioxol-4-yl)methanol (270 mg, 0.683 mmol) in 1,4-dioxane (6 mL) were added 3-bromo-7-iodo-N-(4-methoxybenzyl)quinolin-2-amine (256 mg, 0.546 mmol), 1,10-phenanthroline (19.7 mg, 0.109 mmol), copper(I) iodide (10.4 mg, 0.055 mmol) and cesium carbonate (267 mg, 0.819 mmol). The resulting mixture was stirred at 110° C. for 18 h. The mixture was concentrated under reduced pressure, and the residue was purified by column chromatography on silica (0-30% EtOAc/PE) to afford 3-bromo-N-(4-methoxybenzyl)-7-(((3aR,4R,6R,6aS)-2,2,4-trimethyl-6-(4-phenoxy-7H-pyrrolo[2,3-d]pyrimidin-7-yl)tetrahydro-3aH-cyclopenta[d][1,3]dioxol-4-yl)methoxy)quinolin-2-amine as a solid. MS: 736/738 (M+1/M+3).

Step 11: 3-bromo-N-(4-methoxybenzyl)-7-(((3aR,4R,6R,6aS)-2,2,4-trimethyl-6-(4-phenoxy-7H-pyrrolo[2,3-d]pyrimidin-7-yl)tetrahydro-3aH-cyclopenta[d][1,3]dioxol-4-yl)methoxy)quinolin-2-amine (230 mg, 0.312 mmol) was dissolved in 2,2,2-trifluoroacetic acid (5 mL, 67.3 mmol), then the solution was stirred at 60° C. for 2 h. The solution was co-evaporated with toluene five times, to afford (1S,2R,3R,5R)-3-(((2-amino-3-bromoquinolin-7-yl)oxy)methyl)-3-methyl-5-(4-phenoxy-7H-pyrrolo[2,3-d]pyrimidin-7-yl)cyclopentane-1,2-diol. MS: 576/578 (M+1/M+3).

Step 12: To a solution of (1S,2R,3R,5R)-3-(((2-amino-3-bromoquinolin-7-yl)oxy)methyl)-3-methyl-5-(4-phenoxy-7H-pyrrolo[2,3-d]pyrimidin-7-yl)cyclopentane-1,2-diol (230 mg, 0.299 mmol) in 2-propanol (2 mL) was added ammonia in i-PrOH(NH₃/i-PrOH 5:1) (5 mL, 0.299 mmol) at −50° C., the resulting mixture was stirred at 130° C. for 64 h. The reaction mixture was concentrated under reduced pressure, and the residue was purified by prep-HPLC (ACN/water) to afford (1S,2R,3R,5R)-3-(((2-amino-3-bromoquinolin-7-yl)oxy)methyl)-5-(4-amino-7H-pyrrolo[2,3-d]pyrimidin-7-yl)-3-methylcyclopentane-1,2-diol as a solid. MS: 499/501 (M+1/M+3). 1H-NMR (400 MHz, DMSO-d6) δ 8.29 (s, 1H), 8.03 (s, 1H), 7.59 (d, J=9.2 Hz, 1H), 7.31 (d, J=3.6 Hz, 1H), 6.98-6.92 (m, 2H), 6.89 (br s, 2H), 6.55 (d, J=3.2 Hz, 1H), 6.51 (br s, 2H), 5.03-4.96 (m, 1H), 4.85 (d, J=6.8 Hz, 1H), 4.75 (d, J=4.8 Hz, 1H), 4.56-4.50 (m, 1H), 4.03 (d, J=9.2 Hz, 1H), 3.97-3.91 (m, 2H), 1.93 (d, J=9.6 Hz, 2H), 1.19 (s, 3H).

### Example 101

### (1S,2R,3R,5R)-5-(4-amino-7H-pyrrolo[2,3-d]pyrimidin-7-yl)-3-(((2-aminoquinolin-7-yl)oxy)methyl)-3-methylcyclopentane-1,2-diol

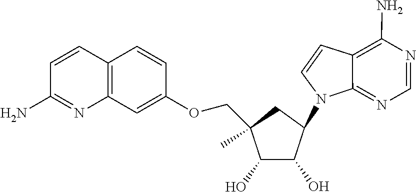

Step 1: To a solution of (1S,2R,3R,5R)-3-(((2-amino-3-bromoquinolin-7-yl)oxy)methyl)-5-(4-amino-7H-pyrrolo[2,3-d]pyrimidin-7-yl)-3-methylcyclopentane-1,2-diol (26 mg, 0.052 mmol) in MeOH (3 mL) was added Pd/C (10%) (20 mg) at 25° C. The mixture was stirred for 30 minutes under a hydrogen atmosphere at 25° C. The mixture was filtered, and the filtrate was purified by reverse phase flash column (ACN/water) to afford (1S,2R,3R,5R)-5-(4-amino-7H-pyrrolo[2,3-d]pyrimidin-7-yl)-3-(((2-aminoquinolin-7-yl)oxy)methyl)-3-methylcyclopentane-1,2-diol as a solid. MS: 421 (M+1). ¹H-NMR (300 MHz, DMSO-d6) δ 8.00 (s, 1H), 7.76 (d, J=9.0 Hz, 1H), 7.50 (d, J=8.7 Hz, 1H), 7.28 (d, J=3.6 Hz, 1H), 6.91-6.80 (m, 4H), 6.57-6.52 (m, 2H), 6.26 (br s, 2H), 5.01-4.92 (m, 1H), 4.82 (d, J=6.6 Hz, 1H), 4.71 (d, J=4.8 Hz, 1H), 4.53-4.46 (m, 1H), 3.98 (d, J=9.6 Hz, 1H), 3.91-3.89 (m, 2H), 1.91-1.88 (m, 2H), 1.16 (s, 3H).

### Example 102

### (1S,2R,5R)-3-(2-(2-amino-3-bromoquinolin-7-yl)ethyl)-5-(4-amino-7H-pyrrolo[2,3-d]pyrimidin-7-yl)-3-(fluoromethyl)cyclopentane-1,2-diol

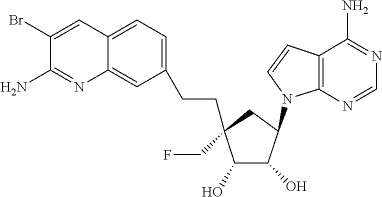 [SMILES: Nc1nc2cc(CC[C@]3(CF)C[C@@H](n4ccc5c(N)ncnc54)[C@H](O)[C@@H]3O)ccc2cc1Br]

Step 1: To a stirred mixture of (1R,2S,3R,5R)-3-(4-chloro-7H-pyrrolo[2,3-d]pyrimidin-7-yl)-5-(hydroxymethyl)cyclopentane-1,2-diol (4.0 g, 14.1 mmol) (azeotroped with toluene 3×) in pyridine (40 mL) was added 1-(chloro(4-methoxyphenyl)(phenyl)methyl)-3-methoxybenzene (5.25 g, 15.5 mmol) at room temperature under an argon atmosphere. The resulting mixture was stirred for about 3 h at room temperature. The reaction mixture was quenched by MeOH (25 mL). The mixture was azeotroped with toluene, and the residue was purified by column chromatography on silica (1-10% MeOH/DCM) to afford (1S,2R,3R,5R)-3-((bis(4-methoxyphenyl)(phenyl)methoxy)methyl)-5-(4-chloro-7H-pyrrolo[2,3-d]pyrimidin-7-yl)cyclopentane-1,2-diol as a solid. MS: 586 (M+1). ¹H-NMR (300 MHz, DMSO-d₆) δ 8.59 (s, 1H), 7.81 (d, J=3.6 Hz, 1H), 7.44-7.41 (m, 2H), 7.35-7.24 (m, 7H), 6.92-6.90 (m, 4H), 6.68 (d, J=3.6 Hz, 1H), 4.98-4.93 (m, 2H), 4.79 (d, J=3.6 Hz, 1H), 4.28-4.26 (m, 1H), 3.89-3.87 (m, 1H), 3.75 (s, 6H), 3.20-3.17 (m, 1H), 3.09-3.05 (m, 1H), 2.32-2.26 (m, 2H), 1.76-1.69 (m, 1H).

Step 2: To a stirred mixture of (1S,2R,3R,5R)-3-((bis(4-methoxyphenyl)(phenyl)methoxy)methyl)-5-(4-chloro-7H-pyrrolo[2,3-d]pyrimidin-7-yl)cyclopentane-1,2-diol (6.0 g, 10.2 mmol) (azeotroped with toluene 3×) in DMF (80 mL) was added sodium hydride (60% in mineral oil) (1.64 g, 41.0 mmol) at 0° C. under an argon atmosphere. The resulting mixture was stirred for about 30 minutes at 0° C., then tetrabutylammonium iodide (1.89 g, 5.12 mmol) and (bromomethyl)benzene (5.25 g, 30.7 mmol) were further added. The resulting mixture was warmed to room temperature and stirred for 3 h. The reaction mixture was quenched by saturated aqueous NH₄Cl (150 mL) and extracted with EtOAc (3×200 mL). The combined organic fractions were washed with brine (3×100 mL), dried over anhydrous sodium sulfate, and filtered. The filtrate was concentrated under reduced pressure, and the residue was purified by column chromatography on silica (0-50% EtOAc/PE) to afford 7-((1R,2S,3R,4R)-2,3-bis(benzyloxy)-4-((bis(4-methoxyphenyl)(phenyl)methoxy)methyl)cyclopentyl)-4-chloro-7H-pyrrolo[2,3-d]pyrimidine as a solid. MS: 766 (M+1). ¹H-NMR (300 MHz, DMSO-d₆) δ 8.46 (s, 1H), 7.75 (d, J=3.9 Hz, 1H), 7.38-7.08 (m, 17H), 6.91-6.84 (m, 6H), 6.61 (d, J=3.6 Hz, 1H), 5.20-5.10 (m, 1H), 4.61-4.48 (m, 2H), 4.39-4.35 (m, 1H), 4.27-4.19 (m, 2H), 3.93-3.91 (m, 1H), 3.71 (s, 6H), 3.15-3.11 (m, 2H), 2.50-2.40 (m, 1H), 2.30-2.20 (m, 1H), 1.82-1.72 (m, 1H).

Step 3: To a stirred solution of 7-((1R,2S,3R,4R)-2,3-bis(benzyloxy)-4-((bis(4-methoxyphenyl)(phenyl)methoxy)methyl)cyclopentyl)-4-chloro-7H-pyrrolo[2,3-d]pyrimidine (6.0 g, 7.83 mmol) in DCM (50 mL) were added water (1.411 g, 78 mmol) and 2,2-dichloroacetic acid (6% DCA in DCM) (9.09 g, 70.5 mmol) at room temperature under an argon atmosphere. The resulting mixture was stirred for 15 minutes at this temperature. Then triethylsilane (18.21 g, 157 mmol) was further added, the resulting mixture was stirred for 50 minutes at this temperature. The reaction mixture was quenched by pyridine (11.2 g, 141 mmol) at 0° C. and stirred for 15 minutes. The mixture was azeotroped with toluene and the residue was purified by column chromatography on silica (0-50% EtOAc/PE) to afford ((1R,2R,3S,4R)-2,3-bis(benzyloxy)-4-(4-chloro-7H-pyrrolo[2,3-d]pyrimidin-7-yl)cyclopentyl)methanol as a solid. MS: 464 (M+1). ¹H-NMR (300 MHz, DMSO-d₆) δ 8.56 (s, 1H), 7.86 (d, J=3.6 Hz, 1H), 7.39-7.27 (m, 5H), 7.15-7.11 (m, 3H), 6.94-6.91 (m, 2H), 6.66 (d, J=3.6 Hz, 1H), 5.36-5.14 (m, 1H), 4.86-4.84 (m, 1H), 4.67-4.52 (m, 2H), 4.45-4.41 (m, 1H), 4.35-4.30 (m, 1H), 4.29-4.25 (m, 1H), 4.03-3.97 (m, 1H), 3.49-3.47 (m, 2H), 2.31-2.27 (m, 2H), 1.74-1.68 (m, 1H).

Step 4: To a stirred solution of ((1R,2R,3S,4R)-2,3-bis(benzyloxy)-4-(4-chloro-7H-pyrrolo[2,3-d]pyrimidin-7-yl)cyclopentyl)methanol (3.1 g, 6.68 mmol) in DCM (25 mL) was added Dess-Martin periodinane (5.67 g, 13.4 mmol) at 0° C. under an argon atmosphere. The resulting mixture was stirred for 15 minutes at 0° C., and the reaction mixture was warmed to room temperature and stirred for 1 h. The reaction mixture was quenched by saturated aqueous NaHCO₃ (20 mL) and extracted with EtOAc (3×100 mL). The combined organic fractions were washed with brine (3×40 mL), dried over anhydrous sodium sulfate, and filtered. The filtrate was concentrated under reduced pressure. The residue was purified by column chromatography on silica (0-50% EtOAc/PE) to afford (1S,2R,3S,4R)-2,3-bis(benzyloxy)-4-(4-chloro-7H-pyrrolo[2,3-d]pyrimidin-7-yl)cyclopentanecarbaldehyde as a solid. MS: 462 (M+1). ¹H-NMR (400 MHz, DMSO-d6) δ 9.79 (s, 1H), 8.57 (s, 1H), 7.85 (d, J=3.6 Hz, 1H), 7.43-7.33 (m, 5H), 7.18-7.13 (m, 3H), 6.96-6.93 (m, 2H), 6.70 (d, J=3.6 Hz, 1H), 5.35-5.28 (m, 1H), 4.75-4.64 (m, 2H), 4.51-4.49 (m, 2H), 4.35-4.23 (m, 2H), 3.32-3.28 (m, 1H), 2.49-2.33 (m, 2H).

Step 5: To a stirred solution of (1S,2R,3S,4R)-2,3-bis(benzyloxy)-4-(4-chloro-7H-pyrrolo[2,3-d]pyrimidin-7-yl)cyclopentanecarbaldehyde (2.4 g, 5.20 mmol) in dioxane (20 mL) were added potassium carbonate (18.8 mL, 37.7 mmol) and formaldehyde (1.01 mL, 5.20 mmol) at 25° C. under an argon atmosphere. The resulting mixture was stirred for 16 h at 25° C. The reaction mixture was quenched by adding HCl aqueous (1 M, 20 mL) to adjust pH=7 at 0° C. The solvent was removed under reduced pressure. The residue was dissolved in ethanol (20 mL), then sodium borohydride (0.236 g, 6.23 mmol) was added at 0° C. under an argon atmosphere. The reaction was stirred at 0° C. for about 2 h. The reaction mixture was quenched by HCl aqueous (1M, 20 mL, to adjust pH=7) at 0° C., water (50 mL) was further added, and the mixture was extracted with EtOAc (3×100 mL). The combined organic fractions were washed with brine (3×50 mL), dried over anhydrous sodium sulfate, and filtered. The filtrate was concentrated under reduced pressure, and the residue was purified by column chromatography on silica (0-80% EtOAc/PE) to afford ((2R,3S,4R)-2,3-bis(benzyloxy)-4-(4-chloro-7H-pyrrolo[2,3-d]pyrimidin-7-yl)cyclopentane-1,1-diyl)dimethanol as a solid. MS: 494 (M+1). ¹H-NMR (300 MHz, DMSO-d₆) δ 8.54 (s, 1H), 7.83 (d, J=3.6 Hz, 1H), 7.38-7.29 (m, 5H), 7.12-7.08 (m, 3H), 6.91-6.88 (m, 2H), 6.64 (d, J=3.6 Hz, 1H), 5.29-5.19 (m, 1H), 4.89-4.85 (m, 1H), 4.73-4.69 (m, 1H), 4.61-4.59 (m, 1H), 4.55-4.49 (m, 1H), 4.48-4.46 (m, 1H), 4.39-4.35 (m, 1H), 4.31-4.27 (m, 1H), 4.09-4.07 (m, 1H), 3.61-3.51 (m, 4H), 2.08-2.00 (m, 1H), 1.71-1.63 (m, 1H).

Step 6: To a stirred solution of ((2R,3S,4R)-2,3-bis(benzyloxy)-4-(4-chloro-7H-pyrrolo[2,3-d]pyrimidin-7-yl)cyclopentane-1,1-diyl)dimethanol (1.2 g, 2.429 mmol) (azeotroped with toluene 3×) in DCM (30 mL) was added triethylamine (0.737 g, 7.29 mmol) at 0° C. under an argon atmosphere. (Chloro(4-methoxyphenyl)methylene)dibenzene (0.788 g, 2.55 mmol) was further added at this temperature, then the reaction mixture was warmed to room temperature and stirred for 2 h. The reaction mixture was quenched by saturated aqueous NH₄Cl (40 mL) and extracted with EtOAc (3×100 mL). The combined organic fractions were washed with brine (3×50 mL), dried over anhydrous sodium sulfate, and filtered. The filtrate was concentrated under reduced pressure, and the residue was purified by column chromatography on silica (0-80% EtOAc/PE) to afford a mixture ((1S,2R,3S,4R)-2,3-bis(benzyloxy)-4-(4-chloro-7H-pyrrolo[2,3-d]pyrimidin-7-yl)-1-(((4-methoxyphenyl)diphenylmethoxy)methyl)cyclopentyl)methanol and ((1R,2R,3S,4R)-2,3-bis(benzyloxy)-4-(4-chloro-7H-pyrrolo[2,3-d]pyrimidin-7-yl)-1-(((4-methoxyphenyl)diphenylmethoxy)methyl)cyclopentyl)methanol as a solid. MS: 766 (M+1). ¹H NMR of the mixture (two regioisomers) (400 MHz, DMSO-d₆) δ 8.57-8.52 (m, 1H), 7.82 (d, J=3.6 Hz, 1H), 7.43-7.24 (m, 15H), 7.13-7.06 (m, 5H), 6.88-6.84 (m, 4H), 6.66 (d, J=3.6 Hz, 1H), 5.14-5.08 (m, 2H), 4.96-4.40 (m, 4H), 4.29-4.12 (m, 2H), 3.77-3.74 (m, 5H), 3.39-3.36 (m, 1H), 3.21-3.19 (m, 1H), 2.09-2.00 (m, 1H), 1.86-1.80 (m, 1H).

Step 7: To a stirred mixture of ((1S,2R,3S,4R)-2,3-bis(benzyloxy)-4-(4-chloro-7H-pyrrolo[2,3-d]pyrimidin-7-yl)-1-(((4-methoxyphenyl)diphenylmethoxy)methyl)cyclopentyl)methanol (1.45 g, 0.473 mmol) and ((1R,2R,3S,4R)-2,3-bis(benzyloxy)-4-(4-chloro-7H-pyrrolo[2,3-d]pyrimidin-7-yl)-1-(((4-methoxyphenyl)diphenylmethoxy)methyl)cyclopentyl)methanol (1.45 g, 1.42 mmol) (azeotroped with toluene 3×) in DMF (15 mL) was added sodium hydride (60% in mineral oil) (0.227 g, 5.68 mmol) at 0° C. under an argon atmosphere. The resulting mixture was stirred for about 30 minutes at 0° C., then tetrabutylammonium iodide (0.262 g, 0.710 mmol) and (bromomethyl)benzene (0.728 g, 4.26 mmol) were further added. The resulting mixture was warmed to room temperature and stirred for 3 hours. The reaction mixture was quenched by saturated aqueous NH₄Cl (20 mL) and extracted with EtOAc (3×100 mL). The combined organic fractions were washed with brine (3×50 mL), dried over anhydrous sodium sulfate, and filtered. The filtrate was concentrated under reduced pressure, and the residue was purified by column chromatography on silica (0-80% EtOAc/PE) to afford a mixture 7-((1R,2S,3R,4S)-2,3-bis(benzyloxy)-4-((benzyloxy)methyl)-4-(((4-methoxyphenyl)diphenylmethoxy)methyl)cyclopentyl)-4-chloro-7H-pyrrolo[2,3-d]pyrimidine as a solid and 7-((1R,2S,3R,4R)-2,3-bis(benzyloxy)-4-((benzyloxy)methyl)-4-(((4-methoxyphenyl)diphenylmethoxy)methyl)cyclopentyl)-4-chloro-7H-pyrrolo[2,3-d]pyrimidine as a solid. MS: 856 (M+1).

Step 8: 7-((1R,2S,3R,4S)-2,3-bis(benzyloxy)-4-((benzyloxy)methyl)-4-(((4-methoxyphenyl)diphenylmethoxy)methyl)cyclopentyl)-4-chloro-7H-pyrrolo[2,3-d]pyrimidine (1.00 g, 0.292 mmol) and 7-((1R,2S,3R,4R)-2,3-bis(benzyloxy)-4-((benzyloxy)methyl)-4-(((4-methoxyphenyl)diphenylmethoxy)methyl)cyclopentyl)-4-chloro-7H-pyrrolo[2,3-d]pyrimidine (1.00 g, 0.876 mmol) was dissolved in AcOH (20 mL) and water (2.5 mL) at room temperature. Then the temperature was raised up to 40° C. and the reaction was stirred for about 4 h. The mixture (azeotroped with toluene 3×) was concentrated under reduced pressure, and the residue was purified by column chromatography on silica (0-80% EtOAc/PE) to afford a mixture of ((1R,2R,3S,4R)-2,3-bis(benzyloxy)-1-((benzyloxy)methyl)-4-(4-chloro-7H-pyrrolo[2,3-d]pyrimidin-7-yl)cyclopentyl)methanol as a solid and ((1S,2R,3S,4R)-2,3-bis(benzyloxy)-1-((benzyloxy)methyl)-4-(4-chloro-7H-pyrrolo[2,3-d]pyrimidin-7-yl)cyclopentyl)methanol as a solid. MS: 584 (M+1). ¹H-NMR of the mixture of two regioisomers (300 MHz, DMSO-d₆) δ 8.51-8.49 (m, 1H), 7.78-7.66 (m, 1H), 7.37-7.26 (m, 10H), 7.11-7.07 (m, 3H), 6.89-6.86 (m, 2H), 6.59-6.54 (m, 1H), 5.26-5.16 (m, 1H), 4.74-4.24 (m, 8H), 4.09-4.07 (m, 1H), 3.66-3.50 (m, 4H), 2.08-1.97 (m, 1H), 1.79-1.71 (m, 1H).

Step 9: To a stirred mixture of ((1S,2R,3S,4R)-2,3-bis(benzyloxy)-1-((benzyloxy)methyl)-4-(4-chloro-7H-pyrrolo[2,3-d]pyrimidin-7-yl)cyclopentyl)methanol (500 mg, 0.642 mmol) and ((1R,2R,3S,4R)-2,3-bis(benzyloxy)-1-((benzyloxy)methyl)-4-(4-chloro-7H-pyrrolo[2,3-d]pyrimidin-7-yl)cyclopentyl)methanol (500 mg, 0.214 mmol) (azeotroped with toluene 3×) in DCM (10 mL) was added DAST (310 mg, 1.93 mmol) at 0° C. under an argon atmosphere. The resulting mixture was stirred for about 16 h at room temperature. The mixture was quenched by saturated aqueous NaHCO₃ (25 mL) and extracted with EtOAc (3×50 mL). The combined organic fractions were washed with brine (3×25 mL), dried over anhydrous sodium sulfate, and filtered. The filtrate was concentrated under reduced pressure, and the residue was purified by Prep-TLC (50% EtOAc/PE) to afford 7-((1R,2S,3R,4S)-2,3-bis(benzyloxy)-4-((benzyloxy)methyl)-4-(fluoromethyl)cyclopentyl)-4-chloro-7H-pyrrolo[2,3-d]pyrimidine as an oil. MS: 586 (M+1). ¹H-NMR (400 MHz, DMSO-d₆) δ 8.51 (s, 1H), 7.74 (d, J=3.6 Hz, 1H), 7.39-7.33 (m, 10H), 7.15-7.06 (m, 3H), 6.90-6.87 (m, 2H), 6.61 (d, J=3.6 Hz, 1H), 5.30-5.20 (m, 1H), 4.80-4.75 (m, 2H), 4.63-4.57 (m, 5H), 4.52-4.48 (m, 1H), 4.33-4.29 (m, 1H), 4.15 (d, J=4.2 Hz, 1H), 3.63-3.61 (m, 2H), 2.10-1.89 (m, 2H).

Step 10: To a stirred mixture of 7-((1R,2S,3R,4S)-2,3-bis(benzyloxy)-4-((benzyloxy)methyl)-4-(fluoromethyl)cyclopentyl)-4-chloro-7H-pyrrolo[2,3-d]pyrimidine (395 mg, 0.674 mmol) (azeotroped with toluene 3×) in DCM (6 mL) was added trichloroborane (1 M in DCM) (6.74 mL, 6.74 mmol) at −80° C. under an argon atmosphere. The resulting mixture was stirred for about 60 minutes at −80° C. The reaction mixture was quenched with saturated aqueous NaHCO₃ (5 mL). The mixture was purified by reverse phase column chromatography (ACN/water with 5 mM NH₄HCO₃ modifier) to afford (1S,2R,3S,5R)-5-(4-chloro-7H-pyrrolo[2,3-d]pyrimidin-7-yl)-3-(fluoromethyl)-3-(hydroxymethyl)cyclopentane-1,2-diol as a solid. MS: 316 (M+1). ¹H-NMR (300 MHz, DMSO-d₆) δ 8.63 (s, 1H), 7.92 (d, J=3.6 Hz, 1H), 6.71 (d, J=3.6 Hz, 1H), 5.18-5.04 (m, 2H), 4.94-4.92 (m, 2H), 4.73-4.34 (m, 3H), 3.90-3.87 (m, 1H), 3.55-3.45 (m, 2H), 2.04-1.96 (m, 1H), 1.70-1.62 (m, 1H).

Step 11: To a stirred mixture of (1S,2R,3S,5R)-5-(4-chloro-7H-pyrrolo[2,3-d]pyrimidin-7-yl)-3-(fluoromethyl)-3-(hydroxymethyl)cyclopentane-1,2-diol (178 mg, 0.564 mmol) (azeotroped with toluene 3×) in acetone (5 mL) was added 4-methylbenzenesulfonic acid (9.71 mg, 0.056 mmol) at 0° C. under an argon atmosphere. The resulting mixture was stirred for about 5 minutes at 0° C., then 2,2-dimethoxypropane (294 mg, 2.82 mmol) was added at this temperature. The resulting mixture was warmed to room temperature and stirred for 3 h. The reaction mixture was quenched by saturated aqueous NaHCO₃ (10 mL) and extracted with EtOAc (3×30 mL). The combined organic fractions were washed with brine (3×10 mL), dried over anhydrous sodium sulfate, and filtered. The filtrate was concentrated under reduced pressure, and the residue was purified by column chromatography on silica (0-80% EtOAc/PE) to afford ((3aR,4S,6R,6aS)-6-(4-chloro-7H-pyrrolo[2,3-d]pyrimidin-7-yl)-4-(fluoromethyl)-2,2-dimethyltetrahydro-3aH-cyclopenta[d][1,3]dioxol-4-yl)methanol as a solid. MS: 356 (M+1). ¹H-NMR (400 MHz, DMSO-d₆) δ 8.67 (s, 1H), 8.01 (d, J=3.6 Hz, 1H), 6.75 (d, J=3.6 Hz, 1H), 5.29-5.23 (m, 1H), 5.18 (t, J=4.2 Hz, 1H), 5.04 (t, J=6.8 Hz, 1H), 4.68-4.60 (m, 2H), 4.56-4.48 (m, 1H), 3.53-3.45 (m, 2H), 2.24-2.15 (m, 2H), 1.50 (s, 3H), 1.24 (s, 3H).

Step 12: To a stirred solution of oxalyl dichloride (0.103 mL, 1.22 mmol) in anhydrous DCM (5 mL) was added (methylsulfinyl)methane (0.174 mL, 2.445 mmol) at −78° C. under an argon atmosphere, the mixture was stirred for 0.5 h at −78° C. Then a solution of ((3aR,4S,6R,6aS)-6-(4-chloro-7H-pyrrolo[2,3-d]pyrimidin-7-yl)-4-(fluoromethyl)-2,2-dimethyltetrahydro-3aH-cyclopenta[d][1,3]dioxol-4-yl)methanol (145 mg, 0.408 mmol) in anhydrous DCM (3 mL) was added dropwise to the mixture at −78° C. The mixture was stirred for another 0.5 h at −78° C. Under this temperature, triethylamine (0.568 mL, 4.08 mmol) was further added to the reaction mixture, and the resulting mixture was stirred for 0.5 h at room temperature. The reaction was quenched with H₂O (10.0 mL) at 0° C. and extracted with DCM (20 mL). The combined organic layers were washed with saturated aqueous NaHCO₃ (10×3 mL), brine (10 mL), dried over anhydrous Na₂SO₄, and filtered. The filtrate was concentrated under reduced pressure to afford (3aR,4R,6R,6aS)-6-(4-chloro-7H-pyrrolo[2,3-d]pyrimidin-7-yl)-4-(fluoromethyl)-2,2-dimethyltetrahydro-3aH-cyclopenta[d][1,3]dioxole-4-carbaldehyde as an oil. MS: 354 (M+1).

Step 13: Bromo(methyl)triphenylphosphorane (367 mg, 1.03 mmol) was dissolved in THF (4 mL) at −30° C. under an argon atmosphere, then butyllithium (2.5 M in hexanes) (0.382 mL, 0.954 mmol) was added dropwise at this temperature. The reaction mixture was stirred at −10° C. for about 30 minutes. Then (3aR,4R,6R,6aS)-6-(4-chloro-7H-pyrrolo[2,3-d]pyrimidin-7-yl)-4-(fluoromethyl)-2,2-dimethyltetrahydro-3aH-cyclopenta[d][1,3]dioxole-4-carbaldehyde (130 mg, 0.367 mmol) in THF (2 mL) was added at −30° C. The reaction was stirred for about 1 h at room temperature. The reaction mixture was quenched by adding saturated aqueous NH₄Cl (10 mL) at −40° C. and extracted with EtOAc (3×30 mL). The combined organic fractions were washed with brine (3×10 mL), dried over anhydrous sodium sulfate, and filtered. The filtrate was concentrated under reduced pressure, and the residue was purified by Prep-TLC (2:1=PE: EtOAc) to afford 4-chloro-7-((3aS,4R,6S,6aR)-6-(fluoromethyl)-2,2-dimethyl-6-vinyltetrahydro-3aH-cyclopenta[d][1,3]dioxol-4-yl)-7H-pyrrolo[2,3-d]pyrimidine. MS: 352 (M+1). ¹H-NMR (300 MHz, DMSO-d₆): δ 8.67 (s, 1H), 8.00 (d, J=3.6 Hz, 1H), 6.73 (d, J=3.6 Hz, 1H), 6.06-5.96 (m, 1H), 5.31-5.08 (m, 4H), 4.79-4.77 (m, 1H), 4.65-4.58 (m, 1H), 4.49-4.42 (m, 1H), 2.49-2.44 (m, 2H), 1.48 (s, 3H), 1.24 (s, 3H).

Step 14: 4-chloro-7-((3aS,4R,6S,6aR)-6-(fluoromethyl)-2,2-dimethyl-6-vinyltetrahydro-3aH-cyclopenta[d][1,3]dioxol-4-yl)-7H-pyrrolo[2,3-d]pyrimidine (100 mg, 0.284 mmol) was dissolved in 5 mL dioxane and ammonia hydrate (28% in water)) (5 mL, 36.4 mmol) in a sealed tube. The reaction was stirred at 90° C. for about 16 h. The mixture was concentrated under reduced pressure, and the residue was purified by reverse phase column chromatography (ACN/water with 5 mM NH₄HCO₃ modifier) to afford 7-((3aS,4R,6S,6aR)-6-(fluoromethyl)-2,2-dimethyl-6-vinyltetrahydro-3aH-cyclopenta[d][1,3]dioxol-4-yl)-7H-pyrrolo[2,3-d]pyrimidin-4-amine as a solid. MS: 333 (M+1). ¹H NMR (300 MHz, DMSO-d₆): δ 8.07 (s, 1H), 7.36 (d, J=3.6 Hz, 1H), 6.99 (s, 2H), 6.58 (d, J=3.6 Hz, 1H), 6.07-5.98 (m, 1H), 5.26-5.17 (m, 3H), 5.08-5.04 (m, 1H), 4.77-4.74 (m, 1H), 4.68-4.52 (m, 1H), 4.49-4.42 (m, 1H), 2.39-2.35 (m, 2H), 1.48 (s, 3H), 1.25 (s, 3H).

Step 15: Under an argon atmosphere, 7-((3aS,4R,6S,6aR)-6-(fluoromethyl)-2,2-dimethyl-6-vinyltetrahydro-3aH-cyclopenta[d][1,3]dioxol-4-yl)-7H-pyrrolo[2,3-d]pyrimidin-4-amine (20 mg, 0.060 mmol) was dissolved in 9-BBN (0.5 M in THF, 0.602 mL, 0.301 mmol) at room temperature, and the mixture was stirred for 1 h at 60° C. The mixture was cooled to 0° C., and a solution of K₃PO₄ (63.9 mg, 0.301 mmol) in 0.30 mL H₂O was added. The mixture was stirred for 0.5 h at room temperature. Then a solution of 3-bromo-7-iodo-N-(4-methoxybenzyl)quinolin-2-amine (28.2 mg, 0.060 mmol) in 0.90 mL anhydrous THF and Pd(dppf)Cl₂ (7.37 mg, 9.03 μmol) were added to the mixture respectively. The resulting mixture was irradiated with microwave radiation at 70° C. for 3 h. The organic layer was separated and concentrated under reduced pressure. The crude product was purified by Prep-TLC (DCM/MeOH=15:1) to afford 7-(2-((3aR,6R,6aS)-6-(4-amino-7H-pyrrolo[2,3-d]pyrimidin-7-yl)-4-(fluoromethyl)-2,2-dimethyltetrahydro-3aH-cyclopenta[d][1,3]dioxol-4-yl)ethyl)-3-bromo-N-(4-methoxybenzyl)quinolin-2-amine as a solid. MS: 675/677 (M+1/M+3).

Step 16: Under an argon atmosphere, 7-(2-((3aR,6R,6aS)-6-(4-amino-7H-pyrrolo[2,3-d]pyrimidin-7-yl)-4-(fluoromethyl)-2,2-dimethyltetrahydro-3aH-cyclopenta[d][1,3]dioxol-4-yl)ethyl)-3-bromo-N-(4-methoxybenzyl)quinolin-2-amine (45 mg, 0.067 mmol) was dissolved in a solution of TFA in H₂O (2.0 mL, TFA/H₂O=1:1) at ambient temperature. The reaction mixture was stirred for 1 h at 50° C. The reaction mixture was azeotroped with toluene 3× to remove TFA. The crude product was purified by reverse phase column chromatography (ACN/water with 5 mM NH₄HCO₃ modifier). The product was further purified by Prep-HPLC (ACN/water with 6.3 mM NH₄HCO₃ modifier) afford desired product (1S,2R,5R)-3-(2-(2-amino-3-bromoquinolin-7-yl)ethyl)-5-(4-amino-7H-pyrrolo[2,3-d]pyrimidin-7-yl)-3-(fluoromethyl)cyclopentane-1,2-diol as a solid. MS: 515/517 (M+1/M+3). ¹H-NMR (300 MHz, DMSO-d₆) δ 8.34 (s, 1H), 8.04 (s, 1H), 7.60 (d, J=8.1 Hz, 1H), 7.36 (s, 1H), 7.28 (d, J=3.6 Hz, 1H), 7.17-7.14 (m, 1H), 6.92 (br s, 2H), 6.58-6.53 (m, 3H), 4.95-4.83 (m, 3H), 4.79-4.38 (m, 3H), 3.85 (t, J=4.2 Hz, 1H), 2.86-2.69 (m, 2H), 2.01-1.76 (m, 4H).

### Example 103

### (1R,2S,3R,5S)-5-(2-(2-amino-3-bromoquinolin-7-yl)ethyl)-3-(4-amino-7H-pyrrolo[2,3-d]pyrimidin-7-yl)-1-methylcyclopentane-1,2-diol

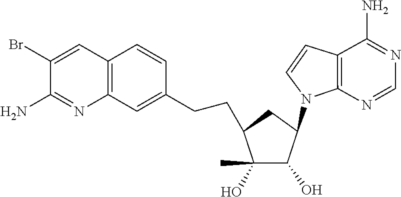 [SMILES: C[C@@]1(O)[C@@H](CCc2ccc3cc(Br)c(N)nc3c2)C[C@@H](n2ccc3c(N)ncnc32)[C@@H]1O]

Step 1: Under an argon atmosphere, to a solution of oxalyl dichloride (1015 mg, 7.99 mmol) in anhydrous DCM (10 mL) was added DMSO (1249 mg, 15.99 mmol) at −78° C. The mixture was stirred for 0.5 h at −78° C. Then a solution of ((3aR,4R,6R,6aS)-6-(4-chloro-7H-pyrrolo[2,3-d]pyrimidin-7-yl)-2,2,3a-trimethyltetrahydro-3aH-cyclopenta[d][1,3]dioxol-4-yl)methanol (900 mg, 2.66 mmol) in anhydrous DCM (10 mL) was added dropwise to the mixture at −78° C. The mixture was stirred for another 0.5 h at −78° C. Under this temperature, TEA (2.70 mg, 26.6 mmol) was added to the reaction mixture, and the resulting mixture was stirred for 0.5 h at −78° C. The reaction was quenched by H₂O (10 mL) at 0° C. The mixture was extracted with DCM (50 mL), washed with saturated aqueous NaHCO₃ (3×30 mL), brine (30 mL). The combined organic layers were dried over anhydrous Na₂SO₄ and filtered. The filtrate was concentrated under reduced pressure to afford the crude product (3aR,4S,6R,6aS)-6-(4-chloro-7H-pyrrolo[2,3-d]pyrimidin-7-yl)-2,2,3a-trimethyltetrahydro-3aH-cyclopenta[d][1,3]dioxole-4-carbaldehyde as a solid. MS: 336 (M+1).

Step 2: To a stirred solution of methyltriphenylphosphonium bromide (2.7 g, 7.50 mmol) in anhydrous THF (4.0 mL) was added n-BuLi (2.5 M in THF, 2.79 mL, 6.97 mmol) dropwise at −10° C. under an argon atmosphere. The reaction mixture was stirred for 0.5 h at room temperature. Then a solution of (3aR,4S,6R,6aS)-6-(4-chloro-7H-pyrrolo[2,3-d]pyrimidin-7-yl)-2,2,3a-trimethyltetrahydro-3aH-cyclopenta[d][1,3]dioxole-4-carbaldehyde (900 mg, 2.68 mmol) in anhydrous THF (6.0 mL) was added to the mixture at −10° C. The mixture was stirred for 1.5 h at room temperature. The reaction mixture was diluted with DCM (50 mL) and washed with water (50 mL) and brine (50 mL). The organic layer was dried over anhydrous sodium sulfate and filtered. The filtrate was concentrated under reduced pressure and purified by column chromatography on silica (EtOAc/PE 0-30%) to afford 4-chloro-7-((3aS,4R,6R,6aR)-2,2,6a-trimethyl-6-vinyltetrahydro-3aH-cyclopenta[d][1,3]dioxol-4-yl)-7H-pyrrolo[2,3-d]pyrimidine as a solid. MS: 334 (M+1).

Step 3: Into a sealed tube were added 4-chloro-7-((3aS,4R,6R,6aR)-2,2,6a-trimethyl-6-vinyltetrahydro-3aH-cyclopenta[d][1,3]dioxol-4-yl)-7H-pyrrolo[2,3-d]pyrimidine (800 mg, 2.40 mmol), 1,4-dioxane (12 mL) and NH₃.H₂O (25%, 16 mL) at room temperature. The mixture was sealed tightly and then stirred at 90° C. for 16 h. Then the reaction mixture was concentrated under reduced pressure. The residue was purified by column chromatography on silica (0-10% MeOH/DCM) to afford 7-((3aS,4R,6R,6aR)-2,2,6a-trimethyl-6-vinyltetrahydro-3aH-cyclopenta[d][1,3]dioxol-4-yl)-7H-pyrrolo[2,3-d]pyrimidin-4-amine as a solid. MS: 315 (M+1).

Step 4: To a stirred solution of 7-((3aS,4R,6R,6aR)-2,2,6a-trimethyl-6-vinyltetrahydro-3aH-cyclopenta[d][1,3]dioxol-4-yl)-7H-pyrrolo[2,3-d]pyrimidin-4-amine (100 mg, 0.32 mmol) in anhydrous THF (0.5 mL) was added 9-BBN (0.5 M in THF, 3.18 mL, 1.59 mmol) dropwise at 0° C. under an argon atmosphere. The reaction solution was stirred at 50° C. for 1 h. To the reaction solution was added a solution of potassium phosphate tribasic (336 mg, 1.581 mmol) in water (1 mL) dropwise at 0° C. The reaction solution was stirred at room temperature for 0.5 h. A solution of 3-bromo-7-iodo-N-(4-methoxybenzyl)quinolin-2-amine (163 mg, 0.348 mmol) in THF (1 ml) and 1,1′-bis(diphenylphosphino)ferrocene-palladium(II)dichloride dichloromethane complex (25.8 mg, 0.032 mmol) were added at room temperature. The final reaction mixture was irradiated with microwave radiation at 70° C. for 2 h. The reaction mixture was cooled to room temperature, diluted with water (5 mL), and extracted with EtOAc (2×20 mL). The combined organic layers were dried over anhydrous sodium sulfate and filtered. The filtrate was concentrated under reduced pressure and purified by Combi-flash (Column: AQ-C18 Column, 80 g, 60A, 40-60 μm; Mobile Phase A: water, Mobile Phase B: MeCN; Flow rate: 50 mL/min; Gradient: 20% B to 90% B in 60 min (80% hold 5 min)) to afford 7-(2-((3aR,4S,6R,6aS)-6-(4-amino-7H-pyrrolo[2,3-d]pyrimidin-7-yl)-2,2,3a-trimethyltetrahydro-3aH-cyclopenta[d][1,3]dioxol-4-yl)ethyl)-3-bromo-N-(4-methoxybenzyl)quinolin-2-amine as a solid. MS: 657/659 (M+1/M+3).

Step 5: A solution of 7-(2-((3aR,4S,6R,6aS)-6-(4-amino-7H-pyrrolo[2,3-d]pyrimidin-7-yl)-2,2,3a-trimethyltetrahydro-3aH-cyclopenta[d][1,3]dioxol-4-yl)ethyl)-3-bromo-N-(4-methoxybenzyl)quinolin-2-amine (40 mg, 0.061 mmol) in TFA (2 mL, 26.0 mmol) was stirred at 60° C. for 1 h. TFA was removed under reduced pressure to obtain the crude product N-(7-(2-((1S,2R,3S,4R)-4-(4-amino-7H-pyrrolo[2,3-d]pyrimidin-7-yl)-2,3-dihydroxy-2-methylcyclopentyl)ethyl)-3-bromoquinolin-2-yl)-2,2,2-trifluoroacetamidde as an oil. A mixture of N-(7-(2-((1S,2R,3S,4R)-4-(4-amino-7H-pyrrolo[2,3-d]pyrimidin-7-yl)-2,3-dihydroxy-2-methylcyclopentyl)ethyl)-3-bromoquinolin-2-yl)-2,2,2-trifluoroacetamide (40 mg, 0.067 mmol) and K₂CO₃ (28 mg, 0.202 mmol) in methanol (3 mL) was stirred at 60° C. for 1 h. The mixture was filtered and washed with MeOH (0.5 mL). The filtrate was concentrated under reduced pressure, and the residue was purified by reverse phase column chromatography (ACN/water) to afford (1R,2S,3R,5S)-5-(2-(2-amino-3-bromoquinolin-7-yl)ethyl)-3-(4-amino-7H-pyrrolo[2,3-d]pyrimidin-7-yl)-1-methylcyclopentane-1,2-diol as a solid. MS: 497/499 (M+1/M+3). ¹H-NMR (400 MHz, DMSO-d₆) δ 8.32 (s, 1H), 8.03 (s, 1H), 7.58 (d, J=8.0 Hz, 1H), 7.32 (s, 1H), 7.25 (d, J=3.6 Hz, 1H), 7.11 (d, J=8.0 Hz, 1H), 6.87 (br s, 2H), 6.55-6.54 (m, 3H), 4.85 (d, J=6.8 Hz, 1H), 4.80-4.77 (m, 1H), 4.18 (s, 1H), 3.98-3.94 (m, 1H), 2.78-2.73 (m, 1H), 2.59-2.50 (m, 1H), 2.32-2.27 (m, 1H), 1.91-1.83 (m, 2H), 1.63-1.57 (m, 2H), 1.15 (s, 3H).

### Example 104

### (2R,3R,3aS,6S,6aR)-2-(4-amino-7H-pyrrolo[2,3-d]pyrimidin-7-yl)-6-((2-aminoquinolin-7-yl)oxy)hexahydro-2H-cyclopenta[b]furan-3,3a-diol

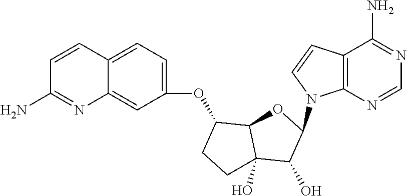 [SMILES: Nc1ccc2ccc(O[C@H]3CC[C@@]4(O)[C@@H]3O[C@@H](n3ccc5c(N)ncnc53)[C@@H]4O)cc2n1]

Step 1: To a solution of (2R,3R,3aS,6S,6aR)-6-((2-amino-3-bromoquinolin-7-yl)oxy)-2-(4-amino-7H-pyrrolo[2,3-d]pyrimidin-7-yl)hexahydro-2H-cyclopenta[b]furan-3,3a-diol (45 mg, 0.088 mmol) in MeOH (6 mL) was added dihydroxypalladium on carbon (18.5 mg, 0.026 mmol) at ambient temperature. The resulting mixture was stirred at 25° C. for 30 minutes under a hydrogen atmosphere (1.2 atm). Then the reaction mixture was filtered, and the filtrate was concentrated under reduced pressure, and the residue was purified by reverse phase HPLC (ACN/water modified with 0.05% TFA). The product-containing fractions were collected, and the pH value of the solution was adjusted to 7˜8 with NH₃.H₂O (25%). Then the solution was concentrated under reduced pressure and further purified by reverse phase column chromatography (ACN/water with 5 mM NH₄HCO₃ modifier) to afford (2R,3R,3aS,6S,6aR)-2-(4-amino-7H-pyrrolo[2,3-d]pyrimidin-7-yl)-6-((2-aminoquinolin-7-yl)oxy)hexahydro-2H-cyclopenta[b]furan-3,3a-diol as a solid. MS: 435 (M+1). ¹H-NMR (300 MHz, DMSO-d₆) δ 8.09 (s, 1H), 7.76 (d, J=9.0 Hz, 1H), 7.52-7.49 (m, 2H), 7.05 (br s, 2H), 6.80-6.75 (m, 2H), 6.66 (d, J=3.3 Hz, 1H), 6.55 (d, J=8.7 Hz, 1H), 6.30 (br s, 2H), 6.02 (d, J=8.4 Hz, 1H), 5.36 (d, J=7.2 Hz, 1H), 5.30 (s, 1H), 4.59 (d, J=4.5 Hz, 1H), 4.40 (t, J=7.8 Hz, 1H), 4.09-4.08 (m, 1H), 2.50-2.48 (m, 1H), 2.07-1.98 (m, 3H).

### Example 105

### (1R,2S,3R,5R)-5-(((2-aminoquinolin-7-yl)oxy)methyl)-1-methyl-3-(7H-pyrrolo[2,3-d]pyrimidin-7-yl)cyclopentane-1,2-diol

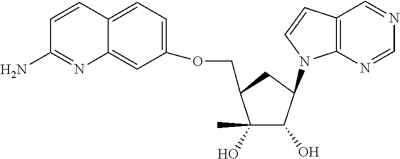 [SMILES: C[C@@]1(O)[C@H](COc2ccc3ccc(N)nc3c2)C[C@@H](n2ccc3cncnc32)[C@@H]1O]

Step 1: To a solution of (1R,2S,3R,5R)-5-(((2-amino-3-bromoquinolin-7-yl)oxy)methyl)-3-(4-chloro-7H-pyrrolo[2,3-d]pyrimidin-7-yl)-1-methylcyclopentane-1,2-diol (20 mg, 0.039 mmol) in MeOH (4 mL) were added triethylamine (7.80 mg, 0.077 mmol) and anhydrous Pd/C (10 mg) (10% Pd/C) under an argon atmosphere. The resulting mixture was stirred at room temperature under a hydrogen atmosphere (˜1 atm) for 4 h. The mixture was filtered, and the filtrate was concentrated under reduced pressure. The residue was purified by reverse phase HPLC (ACN/water with 10 mM NH₄HCO₃ modifier) to afford (1R,2S,3R,5R)-5-(((2-aminoquinolin-7-yl)oxy)methyl)-1-methyl-3-(7H-pyrrolo[2,3-d]pyrimidin-7-yl)cyclopentane-1,2-diol as a solid. MS: 406 (M+1). ¹H-NMR (400 MHz, DMSO-d₆): δ 8.98 (s, 1H), 8.75 (s, 1H), 7.80-7.76 (m, 2H), 7.52 (d, J=8.8 Hz, 1H), 6.94 (d, J=2.4 Hz, 1H), 6.84 (dd, J=8.8, 2.4 Hz, 1H), 6.68 (d, J=3.6 Hz, 1H), 6.58 (d, J=8.8 Hz, 1H), 6.30 (br s, 2H), 5.14 (q, J=9.6 Hz, 1H), 4.95 (d, J=6.8 Hz, 1H), 4.48 (s, 1H), 4.23-4.10 (m, 3H), 2.48-2.37 (m, 2H), 1.80-1.73 (m, 1H), 1.26 (s, 3H).

### Example 106

### (1R,2S,3R,5R)-5-(((2-amino-3-methylquinolin-7-yl)oxy)methyl)-1-methyl-3-(4-methyl-7H-pyrrolo[2,3-d]pyrimidin-7-yl)cyclopentane-1,2-diol

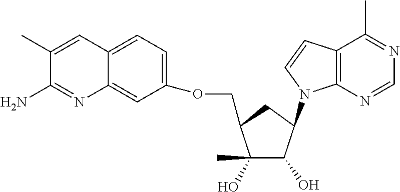 [SMILES: Cc1cc2ccc(OC[C@@H]3C[C@@H](n4ccc5c(C)ncnc54)[C@H](O)[C@]3(C)O)cc2nc1N]

Step 1: To a mixture of (1R,2S,3R,5R)-5-(((2-amino-3-bromoquinolin-7-yl)oxy)methyl)-3-(4-chloro-7H-pyrrolo[2,3-d]pyrimidin-7-yl)-1-methylcyclopentane-1,2-diol (20 mg, 0.039 mmol) and Pd(PPh₃)₄ (4.45 mg, 3.86 μmol) in anhydrous THF (1 mL) was dropwise added trimethylaluminum (2 M in Toluene, 0.058 mL, 0.116 mmol) at room temperature under an argon atmosphere. The mixture was stirred at 100° C. for 2 h. After cooling down to ambient temperature, the mixture was cautiously poured into aqueous HCl (1 M, 10 mL). The mixture was partitioned between EtOAc (40 mL) and H₂O (10 mL). The water layer was concentrated under reduced pressure. The residue was purified by reverse phase HPLC (ACN/water with 10 mM NH₄HCO₃ modifier) to afford (1R,2S,3R,5R)-5-(((2-amino-3-methylquinolin-7-yl)oxy)methyl)-1-methyl-3-(4-methyl-7H-pyrrolo[2,3-d]pyrimidin-7-yl)cyclopentane-1,2-diol as a solid. MS: 434 (M+1). ¹H-NMR (400 MHz, DMSO-d₆): δ 8.60 (s, 1H), 7.71 (d, J=3.6 Hz, 1H), 7.63 (s, 1H), 7.48 (d, J=8.4 Hz, 1H), 6.95 (d, J=2.0 Hz, 1H), 6.84 (dd, J=8.4, 2.0 Hz, 1H), 6.71 (d, J=3.6 Hz, 1H), 6.12 (br s, 2H), 5.10 (q, J=9.6 Hz, 1H), 4.91 (d, J=7.2 Hz, 1H), 4.45 (s, 1H), 4.21-4.09 (m, 3H), 2.64 (s, 3H), 2.46-2.37 (m, 2H), 2.17 (s, 3H), 1.79-1.72 (m, 1H), 1.26 (s, 3H).

### Example 107

### (2R,3R,3aS,6S,6aR)-6-((2-aminoquinolin-7-yl)methyl)-2-(4-methyl-7H-pyrrolo[2,3-d]pyrimidin-7-yl)hexahydro-2H-cyclopenta[b]furan-3,3a-diol

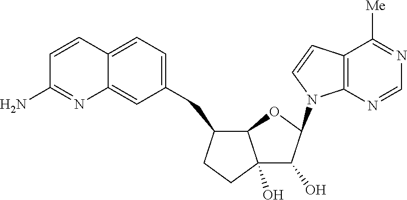 [SMILES: Cc1ncnc2c1ccn2[C@@H]1O[C@@H]2[C@H](Cc3ccc4ccc(N)nc4c3)CC[C@]2(O)[C@H]1O]

Step 1: To a solution of (2R,3R,3aS,6S,6aR)-6-((2-amino-3-bromoquinolin-7-yl)methyl)-2-(4-methyl-7H-pyrrolo[2,3-d]pyrimidin-7-yl)hexahydro-2H-cyclopenta[b]furan-3,3a-diol (20 mg, 0.039 mmol) in MeOH (5 mL) were added triethylamine (3.97 mg, 0.039 mmol) and anhydrous Pd/C (10 mg) (10% Pd/C) under an argon atmosphere. The resulting mixture was stirred at room temperature under hydrogen atmosphere (1.2 atm) for 30 minutes. The mixture was filtered, and the filtrate was concentrated under reduced pressure. The residue was purified by reverse phase HPLC (ACN/water with 10 mM NH₄HCO₃ modifier) to afford (2R,3R,3aS,6S,6aR)-6-((2-aminoquinolin-7-yl)methyl)-2-(4-methyl-7H-pyrrolo[2,3-d]pyrimidin-7-yl)hexahydro-2H-cyclopenta[b]furan-3,3a-diol as a solid. MS: 432 (M+1). ¹H-NMR (400 MHz, DMSO-d₆): δ 8.69 (s, 1H), 7.87 (d, J=3.6 Hz, 1H), 7.79 (d, J=8.8 Hz, 1H), 7.48 (d, J=8.0 Hz, 1H), 7.22 (s, 1H), 7.00 (dd, J=8.0, 1.6 Hz, 1H), 6.82 (d, J=3.6 Hz, 1H), 6.66 (d, J=8.8 Hz, 1H), 6.31 (br s, 2H), 6.01 (d, J=8.0 Hz, 1H), 5.29 (d, J=7.2 Hz, 1H), 5.11 (s, 1H), 4.22 (t, J=7.6 Hz, 1H), 4.01 (d, J=5.6 Hz, 1H), 2.84-2.78 (m, 1H), 2.69 (s, 3H), 2.63-2.58 (m, 1H), 2.28-2.25 (m, 1H), 1.98-1.94 (m, 1H), 1.80-1.68 (m, 2H), 1.58-1.53 (m, 1H).

### Example 108

### (2R,3R,3aS,6S,6aR)-6-((2-amino-3-bromoquinolin-7-yl)methyl)-2-(4-amino-5-fluoro-7H-pyrrolo[2,3-d]pyrimidin-7-yl)hexahydro-2H-cyclopenta[b]furan-3,3a-diol

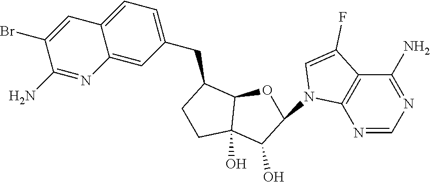 [SMILES: Nc1nc2cc(C[C@@H]3CC[C@@]4(O)[C@@H]3O[C@@H](n3cc(F)c5c(N)ncnc53)[C@@H]4O)ccc2cc1Br]

Step 1: To an oven-dried, argon cooled vial containing (3R,3aS,6aR)-6-((3-bromo-2-((4-methoxybenzyl)amino)quinolin-7-yl)methyl)hexahydro-2H-cyclopenta[b]furan-2,3,3a-triol (132.5 mg, 0.257 mmol) dissolved in dry acetonitrile (2.5 mL) was added 1,1′-(azodicarbonyl)dipiperidine (97 mg, 0.386 mmol) followed by tri-n-butylphosphine (103 μl, 0.411 mmol) at room temperature. The mixture was stirred for 1 h. In a separate oven-dried, argon cooled vial containing 4-chloro-5-fluoro-7H-pyrrolo[2,3-d]pyrimidine (88 mg, 0.514 mmol) dissolved in anhydrous acetonitrile (2.5 mL) was added DBU (78 μl, 0.514 mmol). The mixture was stirred at room temperature for 30 minutes, and this suspension was transferred to the first solution via syringe. The reaction was stirred at room temperature under argon for 7.5 h and then quenched with water and extracted with EtOAc (3×). The combined organics were then washed with brine, dried over sodium sulfate, filtered, and concentrated under reduced pressure. The residue was purified by column chromatography on silica (0-50% 3:1 EtOAc:EtOH in hexanes) to afford (2R,3R,3aS,6aR)-6-((3-bromo-2-((4-methoxybenzyl)amino)quinolin-7-yl)methyl)-2-(4-chloro-5-fluoro-7H-pyrrolo[2,3-d]pyrimidin-7-yl)hexahydro-2H-cyclopenta[b]furan-3,3a-diol as an oil. MS: 668 (M+1).

Step 2: To a vial containing (2R,3R,3aS,6aR)-6-((3-bromo-2-((4-methoxybenzyl)amino)quinolin-7-yl)methyl)-2-(4-chloro-5-fluoro-7H-pyrrolo[2,3-d]pyrimidin-7-yl)hexahydro-2H-cyclopenta[b]furan-3,3a-diol (138 mg, 0.206 mmol), was added ammonia (7 M in MeOH, 2.5 mL, 17.50 mmol). The vial was capped and heated at 140° C. in a microwave reactor for 5 h. The mixture was concentrated under reduced pressure, and the residue was purified by column chromatography on silica gel (0-70% 3:1 EtOAc:EtOH in hexanes) to afford (2R,3R,3aS,6aR)-2-(4-amino-5-fluoro-7H-pyrrolo[2,3-d]pyrimidin-7-yl)-6-((3-bromo-2-((4-methoxybenzyl)amino)quinolin-7-yl)methyl)hexahydro-2H-cyclopenta[b]furan-3,3a-diol as a solid. MS: 649 (M+1).

Step 3: To (2R,3R,3aS,6aR)-2-(4-amino-5-fluoro-7H-pyrrolo[2,3-d]pyrimidin-7-yl)-6-((3-bromo-2-((4-methoxybenzyl)amino)quinolin-7-yl)methyl)hexahydro-2H-cyclopenta[b]furan-3,3a-diol (68 mg, 0.105 mmol) dissolved in DCM (875 μl) was added TFA (348 μl, 4.516 mmol) at room temperature and stirred for 4 h. TFA (500 ul, 6.489 mmol) was added to the reaction mixture and stirred overnight at room temperature. The mixture was heated to 40° C. for 8 h. The reaction mixture was concentrated under reduced pressure and purified by mass trigged reverse phase HPLC (ACN/water with 0.1% NH₄OH modifier) to afford (2R,3R,3aS,6S,6aR)-6-((2-amino-3-bromoquinolin-7-yl)methyl)-2-(4-amino-5-fluoro-7H-pyrrolo[2,3-d]pyrimidin-7-yl)hexahydro-2H-cyclopenta[b]furan-3,3a-diol as a solid. MS: 668 (M+1). 1H NMR (600 MHz, DMSO-d6) δ 8.32 (s, 1H), 8.10 (s, 1H), 7.55 (d, J=8.3 Hz, 1H), 7.51 (d, J=1.7 Hz, 1H), 7.29 (s, 1H), 7.12-6.98 (m, 3H), 6.59 (s, 2H), 5.94 (dd, J=8.2, 1.5 Hz, 1H), 5.28 (d, J=7.0 Hz, 1H), 5.09 (s, 1H), 4.01 (t, J=7.5 Hz, 1H), 3.94 (d, J=5.8 Hz, 1H), 2.85-2.79 (m, 1H), 2.65-2.59 (m, 1H), 2.28-2.20 (m, 1H), 1.95-1.89 (m, 1H), 1.79-1.69 (m, 1H), 1.69-1.62 (m, 1H), 1.54-1.47 (m, 1H).

### Example 109

### (1S,2R,3S,5R)-3-(2-(2-amino-3-bromoquinolin-7-yl)ethyl)-3-methyl-5-(4-methyl-7H-pyrrolo[2,3-d]pyrimidin-7-yl)cyclopentane-1,2-diol

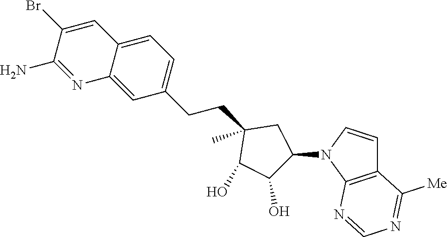 [SMILES: Cc1ncnc2c1ccn2[C@@H]1C[C@](C)(CCc2ccc3cc(Br)c(N)nc3c2)[C@@H](O)[C@H]1O]

Step 1: In a microwave vial was added 4-chloro-7-((3a′R,4′R,6′R,6a'S)-4′-methyl-4′-vinyltetrahydro-4′H-spiro[cyclohexane-1,2′-cyclopenta[d][1,3]dioxol]-6′-yl)-7H-pyrrolo[2,3-d]pyrimidine (0.075 g, 0.2 mmol), solid supported Pd (0.077 g, 0.020 mmol), THF (1 mL), and dimethylzinc (2 M in PhMe, 0.5 mL, 1.0 mmol). The reaction mixture was stirred at room temperature overnight. The reaction was carefully quenched with IPA, then dropwise added MeOH. The reaction mixture was filtered through a plug of Celite, washed with DCM/EtOAc, then concentrated under reduced pressure to afford 4-methyl-7-((3a′R,4′R,6′R,6a'S)-4′-methyl-4′-vinyltetrahydro-4′H-spiro[cyclohexane-1,2′-cyclopenta[d][1,3]dioxol]-6′-yl)-7H-pyrrolo[2,3-d]pyrimidine. MS: 354 (M+1).

Step 2: In a vial was added 4-methyl-7-((3a′R,4′R,6′R,6a'S)-4′-methyl-4′-vinyltetrahydro-3a′H-spiro[cyclohexane-1,2′-cyclopenta[d][1,3]dioxol]-6′-yl)-7H-pyrrolo[2,3-d]pyrimidine (70.7 mg, 0.2 mmol), THF (1 mL), and 9-BBN (0.5 M in THF, 1 mL, 0.500 mmol). The reaction mixture was heated to 50° C. for 2 h, then cooled to room temperature. Another portion of 9-BBN (0.5M in THF, 1 mL, 0.5 mmol) was added and the reaction was heated to 50° C. overnight. The reaction was cooled to room temperature and quenched with K₃PO₄ (2 M in water, 0.75 mL, 1.50 mmol) under an atmosphere of nitrogen gas. In a separate vial was added 3-bromo-N-(2,4-dimethoxybenzyl)-7-iodoquinolin-2-amine (100 mg, 0.20 mmol), 1,1′-Bis(diphenylphosphino)ferrocene]dichloropalladium(II) (16.3 mg, 0.020 mmol) in THF (0.5 mL). This suspension was added to the original reaction vessel, and the mixture was heated to 50° C. overnight. The reaction mixture was cooled to room temperature and diluted with EtOAc, DCM and water. The organic and aqueous layers were separated by passage through a phase separator, and the organic layer was concentrated under reduced pressure to afford 3-bromo-N-(2,4-dimethoxybenzyl)-7-(2-((3a′R,4'S,6′R,6a'S)-4′-methyl-6′-(4-methyl-7H-pyrrolo[2,3-d]pyrimidin-7-yl)tetrahydro-4′H-spiro[cyclohexane-1,2′-cyclopenta[d][1,3]dioxol]-4′-yl)ethyl)quinolin-2-amine. MS: 726/728 (M+1/M+3).

Step 3: A solution of 3-bromo-N-(2,4-dimethoxybenzyl)-7-(2-((3a′R,4'S,6′R,6a'S)-4′-methyl-6′-(4-methyl-7H-pyrrolo[2,3-d]pyrimidin-7-yl)tetrahydro-4′H-spiro[cyclohexane-1,2′-cyclopenta[d][1,3]dioxol]-4′-yl)ethyl)quinolin-2-amine (0.2 mmol) in THF (1 mL), water (0.3 mL, 16.7 mmol), and TFA (0.7 mL, 9.09 mmol) was stirred at room temperature overnight. The reaction mixture was then heated to 50° C. for 3 h. Another portion of TFA (1 mL, 13 mmol) was added at room temperature and the reaction mixture was heated to 50° C. for 3 h. The reaction mixture was concentrated under reduced pressure and purified by mass triggered reverse phase HPLC (ACN/water with 0.1% NH₄OH modifier) to afford (1S,2R,3S,5R)-3-(2-(2-amino-3-bromoquinolin-7-yl)ethyl)-3-methyl-5-(4-methyl-7H-pyrrolo[2,3-d]pyrimidin-7-yl)cyclopentane-1,2-diol as a solid. MS: 496/498 (M+1/M+3). ¹H-NMR (400 MHz, DMSO-d₆) δ 8.29 (s, 1H), 8.03 (s, 1H), 7.59 (d, J=9.2 Hz, 1H), 7.31 (d, J=3.6 Hz, 1H), 6.98-6.92 (m, 2H), 6.89 (br s, 2H), 6.55 (d, J=3.2 Hz, 1H), 6.51 (br s, 2H), 5.03-4.96 (m, 1H), 4.85 (d, J=6.8 Hz, 1H), 4.75 (d, J=4.8 Hz, 1H), 4.56-4.50 (m, 1H), 4.03 (d, J=9.2 Hz, 1H), 3.97-3.91 (m, 2H), 1.93 (d, J=9.6 Hz, 2H), 1.19 (s, 3H).

Example 110: Example 110 in Table 24 was synthesized by using step 2 followed by step 1 & 3 of Example 109. 4-methyl-7-((3a′R,4′R,6′R,6a'S)-4′-methyl-4′-vinyltetrahydro-3a′H-spiro[cyclohexane-1,2′-cyclopenta[d][1,3]dioxol]-6′-yl)-7H-pyrrolo[2,3-d]pyrimidine in step 2 was substituted with an appropriate exo olefin.

TABLE 24

| Ex | Structure | Name | MS |
|---|---|---|---|
| 110 | 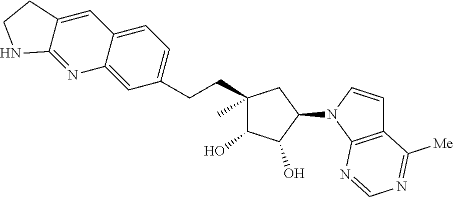 [SMILES: Cc1ncnc2c1ccn2[C@@H]1C[C@](C)(CCc2ccc3cc4c(nc3c2)NCC4)[C@@H](O)[C@H]1O] | (1S,2R,3S,5R)-3-(2-(2,3- dihydro-1H-pyrrolo[2,3- b]quinolin-7-yl)ethyl)-3- methyl-5-(4-methyl-7H- pyrrolo[2,3-d]pyrimidin-7- yl)cyclopentane-1,2-diol | 444 (M + 1) |

### Example 111 (1R,2S,3R,5R)-5-(((2-amino-3-bromoquinolin-7-yl)oxy)methyl)-3-(4-amino-7H-pyrrolo[2,3-d]pyrimidin-7-yl)-1,5-dimethylcyclopentane-1,2-diol

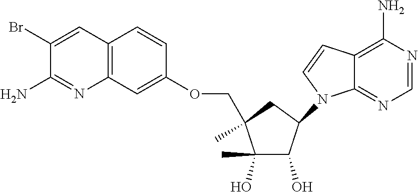 [SMILES: C[C@@]1(COc2ccc3cc(Br)c(N)nc3c2)C[C@@H](n2ccc3c(N)ncnc32)[C@H](O)[C@]1(C)O]

Step 1: A vial was charged with a mixture of 3-bromo-N-(4-methoxybenzyl)-7-(((3aR,4R,6R,6aS)-2,2,4-trimethyl-6-(4-phenoxy-7H-pyrrolo[2,3-d]pyrimidin-7-yl)tetrahydro-3aH-cyclopenta[d][1,3]dioxol-4-yl)methoxy)quinolin-2-amine (100 mg, 0.136 mmol) in 50% aqueous TFA (1 mL). The reaction mixture was stirred at room temperature for 1 h. The solvent was removed under reduced pressure, and the residue was purified by prep-TLC (DCM:MeOH=17:1) to afford (1S,2R,3R,5R)-3-(((3-bromo-2-((4-methoxybenzyl)amino)quinolin-7-yl)oxy)methyl)-3-methyl-5-(4-phenoxy-7H-pyrrolo[2,3-d]pyrimidin-7-yl)cyclopentane-1,2-diol as a solid. MS: 696/698 (M+1/M+3).

Step 2: To a stirred solution of (1S,2R,3R,5R)-3-(((3-bromo-2-((4-methoxybenzyl)amino)quinolin-7-yl)oxy)methyl)-3-methyl-5-(4-phenoxy-7H-pyrrolo[2,3-d]pyrimidin-7-yl)cyclopentane-1,2-diol (60 mg, 0.086 mmol), N-ethyl-N-isopropylpropan-2-aminium chloride (0.428 mg, 2.58 μmol) and (2R,4S)-4-isopropyl-2-methoxy-3-((R)-2-methyl-1-(1-methyl-1H-imidazol-2-yl)propyl)oxazolidine (49 mg, 0.172 mmol) in THF (0.2 mL) were added DIEA (0.045 mL, 0.258 mmol) and tert-butyldimethylsilyl trifluoromethanesulfonate (34.2 mg, 0.129 mmol) at 0° C. under an argon atmosphere. The resulting mixture was stirred at room temperature for 2 h. The reaction was quenched with 10 mL water and extracted with EtOAc (15 mL×3). The organic layer was dried over anhydrous sodium sulfate, filtered, and the filtrate was concentrated under reduced pressure. The residue was purified by prep-TLC (PE/EtOAc=3:1 to afford (1R,2R,4R,5S)-2-(((3-bromo-2-((4-methoxybenzyl)amino)quinolin-7-yl)oxy)methyl)-5-((tert-butyldimethylsilyl)oxy)-2-methyl-4-(4-phenoxy-7H-pyrrolo[2,3-d]pyrimidin-7-yl)cyclopentanol as an oil. MS: 810/812 (M+1/M+3). ¹H-NMR (300 MHz, Chloroform-d) δ 8.46 (s, 1H), 8.05 (s, 1H), 7.50-7.44 (m, 3H), 7.40-7.38 (m, 2H), 7.33-7.30 (m, 1H), 7.29-7.25 (m, 4H), 6.99-6.91 (m, 3H), 6.54 (d, J=3.6 Hz, 1H), 5.60-5.58 (m, 1H), 5.31-5.22 (m, 1H), 4.91-4.86 (m, 1H), 4.77 (s, 2H), 4.08-4.04 (m, 3H), 3.84 (s, 3H), 2.83-2.82 (m, 1H), 2.49-2.41 (m, 1H), 2.22-2.14 (m, 1H), 1.34 (s, 3H), 0.79 (s, 9H),−0.14 (s, 3H), −0.38 (s, 3H).

Step 3: To a stirred solution of (1R,2R,4R,5S)-2-(((3-bromo-2-((4-methoxybenzyl)amino)quinolin-7-yl)oxy)methyl)-5-((tert-butyldimethylsilyl)oxy)-2-methyl-4-(4-phenoxy-7H-pyrrolo[2,3-d]pyrimidin-7-yl)cyclopentanol (20 mg, 0.025 mmol) in DCM (0.5 mL) was added Dess-Martin Periodinane (20.92 mg, 0.049 mmol) in one portion at 0° C. under an argon atmosphere. The mixture was stirred at 0° C. for 2 h. The reaction was quenched with 5 mL saturated aqueous Na₂S₂O₃ and extracted with EtOAc (5 mL×3). The organic layer was dried over anhydrous sodium sulfate, filtered, and the filtrate was concentrated under reduced pressure to afford (2R,4R,5S)-2-(((3-bromo-2-((4-methoxybenzyl)amino)quinolin-7-yl)oxy)methyl)-5-((tert-butyldimethylsilyl)oxy)-2-methyl-4-(4-phenoxy-7H-pyrrolo[2,3-d]pyrimidin-7-yl)cyclopentanone. MS: 808/810 (M+1/M+3)

Step 4: A portion of cerium (III) chloride (37.0 mg, 0.150 mmol) was dried at 140° C. in vacuum for 1 h. The resulting powder was cooled under argon. Dry THF (0.25 mL) was added and then to the mixture was added methyllithium (1.6 M in ether, 0.094 mL, 0.150 mmol) at −78° C. The mixture was stirred at this temperature for 1 h. Then a cooled solution of (2R,4R,5S)-2-(((3-bromo-2-((4-methoxybenzyl)amino)quinolin-7-yl)oxy)methyl)-5-((tert-butyldimethylsilyl)oxy)-2-methyl-4-(4-phenoxy-7H-pyrrolo[2,3-d]pyrimidin-7-yl)cyclopentanone (20.22 mg, 0.025 mmol) in THF (0.25 mL) was rapidly added, and the resulting mixture was kept stirring at this temperature for 6 h. The reaction was quenched with saturated aqueous NH₄Cl (5 mL), and the mixture was extracted with EtOAc (5 mL×3). The organic layer was dried over anhydrous sodium sulfate, filtered, and the filtrate was concentrated under reduced pressure. The residue was purified by prep-TLC (PE/EA=4:1) to afford (1R,2R,4R,5S)-2-(((3-bromo-2-((4-methoxybenzyl)amino)quinolin-7-yl)oxy)methyl)-5-((tert-butyldimethylsilyl)oxy)-1,2-dimethyl-4-(4-phenoxy-7H-pyrrolo[2,3-d]pyrimidin-7-yl)cyclopentanol as an oil. MS: 824/826 (M+1/M+3). ¹H-NMR (400 MHz, Chloroform-d) δ 8.41 (s, 1H), 8.08 (s, 1H), 7.53 (d, J=8.8 Hz, 1H), 7.49-7.44 (m, 3H), 7.41-7.38 (m, 2H), 7.28-7.24 (m, 4H), 6.98-6.93 (m, 4H), 6.52 (d, J=3.2 Hz, 1H), 5.63-5.60 (m, 1H), 5.43-5.36 (m, 1H), 4.86 (d, J=8.8 Hz, 1H), 4.79 (s, 2H), 4.17 (d, J=9.2 Hz, 1H), 3.97 (d, J=9.2 Hz, 1H), 3.84 (s, 3H), 2.40-2.32 (m, 2H), 1.27 (s, 3H), 1.22 (s, 3H), 0.78 (s, 9H), 0.03 (s, 3H), −0.04 (s, 3H).

Step 5: To a stirred solution of (1R,2R,4R,5S)-2-(((3-bromo-2-((4-methoxybenzyl)amino)quinolin-7-yl)oxy)methyl)-5-((tert-butyldimethylsilyl)oxy)-1,2-dimethyl-4-(4-phenoxy-7H-pyrrolo[2,3-d]pyrimidin-7-yl)cyclopentanol (35 mg, 0.042 mmol) in THF (2 mL) was added TBAF (1 M in THF, 0.084 mL, 0.084 mmol) at 0° C. The mixture was stirred at room temperature for 3 h. The reaction mixture was quenched by saturated aqueous NH₄Cl (20 mL) and extracted with EtOAc (30 mL×3). The combined organic layers were washed with brine (20 mL), dried over anhydrous sodium sulfate, and filtered. The filtrate was concentrated under reduced pressure and purified by prep-TLC (PE:EA=1:1) to afford (1R,2S,3R,5R)-5-(((3-bromo-2-((4-methoxybenzyl)amino)quinolin-7-yl)oxy)methyl)-1,5-dimethyl-3-(4-phenoxy-7H-pyrrolo[2,3-d]pyrimidin-7-yl)cyclopentane-1,2-diol as a solid. MS: 710/712 (M+1/M+3). ¹H-NMR (400 MHz, DMSO-d₆) δ 8.31 (s, 1H), 8.24 (s, 1H), 7.67 (d, J=3.6 Hz, 1H), 7.60 (d, J=8.8 Hz, 1H), 7.49-7.45 (m, 2H), 7.35-7.25 (m, 5H), 7.15-7.12 (m, 1H), 7.07 (s, 1H), 6.94 (d, J=8.4 Hz, 1H), 6.88 (d, J=8.0 Hz, 2H), 6.52-6.51 (m, 1H), 5.19-5.14 (m, 1H), 4.99 (d, J=7.6 Hz, 1H), 4.67 (d, J=6.0 Hz, 2H), 4.56 (t, J=8.8 Hz, 1H), 4.38 (s, 1H), 4.14-4.11 (m, 1H), 3.96-3.94 (m, 1H), 3.71 (s, 3H), 2.17-2.11 (m, 1H), 2.05-2.02 (m, 1H), 1.19-1.16 (m, 6H).

Step 6: A solution of (1R,2S,3R,5R)-5-(((3-bromo-2-((4-methoxybenzyl)amino)quinolin-7-yl)oxy)methyl)-1,5-dimethyl-3-(4-phenoxy-7H-pyrrolo[2,3-d]pyrimidin-7-yl)cyclopentane-1,2-diol (17 mg, 0.024 mmol) in TFA (1 mL) was stirred at 50° C. for 3 h. The reaction was concentrated under reduced pressure, and the residue was purified by prep-TLC (DCM:MeOH=12:1) to afford (1R,2S,3R,5R)-5-(((2-amino-3-bromoquinolin-7-yl)oxy)methyl)-1,5-dimethyl-3-(4-phenoxy-7H-pyrrolo[2,3-d]pyrimidin-7-yl)cyclopentane-1,2-diol as a solid. MS: 590/592 (M+1/M+3). ¹H-NMR (400 MHz, DMSO-d₆) δ 8.29 (s, 1H), 8.21 (s, 1H), 7.66 (d, J=3.6 Hz, 1H), 7.60 (d, J=8.8 Hz, 1H), 7.50-7.45 (m, 2H), 7.31-7.28 (m, 1H), 7.26-7.24 (m, 2H), 7.03-7.02 (m, 1H), 6.96-6.94 (m, 1H), 6.54 (br s, 2H), 6.52 (d, J=3.6 Hz, 1H), 5.15 (q, J=9.6 Hz, 1H), 5.00 (d, J=7.2 Hz, 1H), 4.57-4.53 (m, 1H), 4.39 (s, 1H), 4.13 (d, J=9.6 Hz, 1H), 3.93 (d, J=9.6 Hz, 1H), 2.21-2.02 (m, 2H), 1.17-1.16 (m, 6H).

Step 7: (1R,2S,3R,5R)-5-(((2-amino-3-bromoquinolin-7-yl)oxy)methyl)-1,5-dimethyl-3-(4-phenoxy-7H-pyrrolo[2,3-d]pyrimidin-7-yl)cyclopentane-1,2-diol (13 mg, 0.022 mmol) was dissolved in NH₃ (liquid)/i-PrOH (v: v=5:1)) (10 mL) at ambient temperature in a sealed tube. Then the reaction mixture was stirred at 130° C. for 48 h. The reaction mixture was concentrated under reduced pressure and purified by reverse phase HPLC (ACN/water with 10 mmol NH₄HCO₃ modifier) to afford (1R,2S,3R,5R)-5-(((2-amino-3-bromoquinolin-7-yl)oxy)methyl)-3-(4-amino-7H-pyrrolo[2,3-d]pyrimidin-7-yl)-1,5-dimethylcyclopentane-1,2-diol as a solid. MS: 513/515 (M+1/M+3). ¹H-NMR (400 MHz, DMSO-d₆) δ 8.30 (s, 1H), 7.93 (s, 1H), 7.60 (d, J=8.8 Hz, 1H), 7.27 (d, J=3.2 Hz, 1H), 7.01 (s, 1H), 6.96-6.85 (m, 3H), 6.60-6.51 (m, 3H), 5.02-4.95 (m, 2H), 4.49 (d, J=8.8 Hz, 1H), 4.30 (s, 1H), 4.26 (d, J=9.2 Hz, 1H), 4.12 (d, J=9.2 Hz, 1H), 2.01-1.94 (m, 2H), 1.21 (s, 6H).

### Examples 112 and 113

### (2R,3R,3aS,6S,6aR)-6-((2-amino-3-chloroquinolin-7-yl)oxy)-2-(4-methyl-7H-pyrrolo[2,3-d]pyrimidin-7-yl)hexahydro-2H-cyclopenta[b]furan-3,3a-diol & (2R,3R,3aS,6S,6aR)-6-((2-aminoquinolin-7-yl)oxy)-2-(4-methyl-7H-pyrrolo[2,3-d]pyrimidin-7-Yl)hexahydro-2H-cyclopenta[b]furan-3,3a-diol

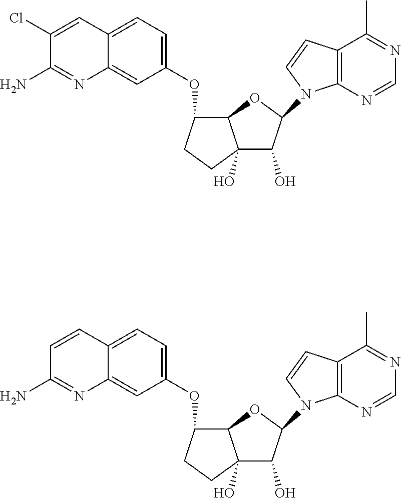 [SMILES: Cc1ncnc2c1ccn2[C@@H]1O[C@@H]2[C@@H](Oc3ccc4cc(Cl)c(N)nc4c3)CC[C@]2(O)[C@H]1O ; Cc1ncnc2c1ccn2[C@@H]1O[C@@H]2[C@@H](Oc3ccc4ccc(N)nc4c3)CC[C@]2(O)[C@H]1O]

Step 1: To a stirred solution of (2R,3R,3aS,6S,6aR)-6-((2-amino-3-bromoquinolin-7-yl)oxy)-2-(4-methyl-7H-pyrrolo[2,3-d]pyrimidin-7-yl)hexahydro-2H-cyclopenta[b]furan-3,3a-diol (200 mg, 0.390 mmol) in EtOH (20 mL) were added monocopper(I) monocopper(III) monooxide (84 mg, 0.586 mmol), tetramethylammonium chloride (428 mg, 3.90 mmol) and (S)-pyrrolidine-2-carboxylic acid (135 mg, 1.17 mmol) at ambient temperature. The mixture was stirred at 110° C. for overnight. The resulting mixture was concentrated under reduced pressure, and the residue was purified by reverse phase column chromatography (ACN/water with 5 mM NH₄HCO₃ modifier) to afford (2R,3R,3aS,6S,6aR)-6-((2-amino-3-chloroquinolin-7-yl)oxy)-2-(4-methyl-7H-pyrrolo[2,3-d]pyrimidin-7-yl)hexahydro-2H-cyclopenta[b]furan-3,3a-diol as a solid. MS: 468 (M+1). ¹H-NMR (400 MHz, Methanol-d₄) δ 8.69 (s, 1H), 8.00 (s, 1H), 7.75 (d, J=3.6 Hz, 1H), 7.55 (d, J=8.8 Hz, 1H), 6.99-6.94 (m, 2H), 6.82 (d, J=3.6 Hz, 1H), 6.18 (d, J=8.0 Hz, 1H), 4.78 (d, J=4.8 Hz, 1H), 4.64 (d, J=8.0 Hz, 1H), 4.35-4.33 (m, 1H), 2.76 (s, 3H), 2.59-2.49 (m, 1H), 2.28-2.19 (m, 3H). ¹H-NMR (400 MHz, DMSO-d₆) δ 8.70 (s, 1H), 8.09 (s, 1H), 7.96 (d, J=3.6 Hz, 1H), 7.57 (d, J=8.8 Hz, 1H), 6.88-6.84 (m, 3H), 6.62 (br s, 2H), 6.15 (d, J=8.4 Hz, 1H), 5.46 (d, J=7.2 Hz, 1H), 5.40 (s, 1H), 4.64 (d, J=5.6 Hz, 1H), 4.48 (t, J=7.6 Hz, 1H), 4.13-4.12 (m, 1H), 2.69 (s, 3H), 2.56-2.54 (m, 1H), 2.08-2.01 (m, 3H). Also, to afford (2R,3R,3aS,6S,6aR)-6-((2-aminoquinolin-7-yl)oxy)-2-(4-methyl-7H-pyrrolo[2,3-d]pyrimidin-7-yl)hexahydro-2H-cyclopenta[b]furan-3,3a-diol as a solid. MS: 434 (M+1). ¹H-NMR (400 MHz, Methanol-d₄) δ 8.66 (s, 1H), 7.80 (d, J=8.8 Hz, 1H), 7.73 (d, J=3.6 Hz, 1H), 7.52 (d, J=8.8 Hz, 1H), 6.95 (d, J=2.0 Hz, 1H), 6.88 (dd, J=8.4, 2.4 Hz, 1H), 6.81 (d, J=3.6 Hz, 1H), 6.63 (d, J=8.4 Hz, 1H), 6.16 (d, J=8.0 Hz, 1H), 4.76 (d, J=5.2 Hz, 1H), 4.62 (d, J=8.0 Hz, 1H), 4.32 (s, 1H), 2.75 (s, 3H), 2.57-2.47 (m, 1H), 2.26-2.18 (m, 3H). ¹H-NMR (400 MHz, DMSO-d₆) δ 8.69 (s, 1H), 7.95 (d, J=3.6 Hz, 1H), 7.76 (d, J=8.8 Hz, 1H), 7.51 (d, J=8.4 Hz, 1H), 6.84-6.76 (m, 3H), 6.55 (d, J=8.8 Hz, 1H), 6.30 (br s, 2H), 6.15 (d, J=8.4 Hz, 1H), 5.46 (d, J=7.2 Hz, 1H), 5.39 (s, 1H), 4.62 (d, J=5.2 Hz, 1H), 4.47 (t, J=7.6 Hz, 1H), 4.13 (s, 1H), 2.69 (s, 3H), 2.56-2.54 (m, 1H), 2.08-1.97 (m, 3H).

### Example 114

### (2R,3R,3aS,6S,6aR)-6-((2-amino-3-bromoquinolin-7- yl)oxy)-2-(2-amino-4-methyl-7H-pyrrolo[2,3-d]pyrimidin-7-yl)hexahydro-3aH-cyclopenta[b]furan-3,3a-diol

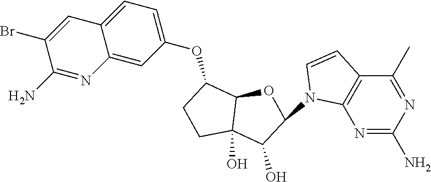 [SMILES: Cc1nc(N)nc2c1ccn2[C@@H]1O[C@@H]2[C@@H](Oc3ccc4cc(Br)c(N)nc4c3)CC[C@]2(O)[C@H]1O]

Step 1: To an oven-dried, argon cooled 2-5 mL microwave vial containing (3R,3aS,6S,6aR)-6-((2-amino-3-bromoquinolin-7-yl)oxy)hexahydro-3aH-cyclopenta[b]furan-2,3,3a-triol (0.086 g, 0.217 mmol) dissolved in anhydrous Acetonitrile (4.3 mL) was added 1,1′-(azodicarbonyl)dipiperidine (0.082 g, 0.325 mmol) followed by tri-n-butylphosphine (0.087 mL, 0.346 mmol) at room temperature. The mixture was stirred for 1 h, and then this solution was used directly without characterization because the product is unstable. In a separate oven-dried, argon cooled microwave vial containing di-tert-butyl (4-methyl-7H-pyrrolo[2,3-d]pyrimidin-2-yl)carbamate (0.151 g, 0.433 mmol) dissolved in dry acetonitrile (1.0 mL) was added DBU (0.065 mL, 0.433 mmol). The mixture was stirred at room temperature for 30 min, and this suspension was transferred to the mixture described above via syringe. The combined reaction was stirred at room temperature under argon for 4 h. The reaction was quenched with water and extracted with EtOAc (3×), the organic layer washed with brine and dried with sodium sulfate, filtered, and concentrated under reduced pressure. The residue was purified by column chromatography (0-100% EtOAc/Hex) to afford di-tert-butyl (7-{(2R,3R,3aS,6S,6aR)-6-[(2-amino-3-bromoquinolin-7-yl)oxy]-3,3a-dihydroxyhexahydro-2H-cyclopenta[b]furan-2-yl}-4-methyl-7H-pyrrolo[2,3-d]pyrimidin-2-yl)imidodicarbonate. MS: 727/729 (M+1/M+3).

Step 2: To di-tert-butyl (7-{(2R,3R,3aS,6S,6aR)-6-[(2-amino-3-bromoquinolin-7-yl)oxy]-3,3a-dihydroxyhexahydro-2H-cyclopenta[b]furan-2-yl}-4-methyl-7H-pyrrolo[2,3-d]pyrimidin-2-yl)imidodicarbonate (0.04 g, 0.055 mmol) dissolved in DCM (1.10 mL) was added 2,2,2-trifluoroacetic acid (0.127 mL, 1.649 mmol). The mixture was stirred for 6 h at room temperature. The mixture was concentrated under reduced pressure and purified by mass triggered reverse phase HPLC (ACN/water modified with 0.10% NH₄OH) to afford (2R,3R,3aS,6S,6aR)-6-((2-amino-3-bromoquinolin-7-yl)oxy)-2-(2-amino-4-methyl-7H-pyrrolo[2,3-d]pyrimidin-7-yl)hexahydro-3aH-cyclopenta[b]furan-3,3a-diol as a TFA salt, as a solid. MS: 527/529 (M+1/M+3). ¹H NMR (499 MHz, DMSO-d₆) δ 9.55-9.19 (m, 3H), 9.15 (d, J=15.5 Hz, 1H), 8.42 (d, J=13.9 Hz, 1H), 8.05 (d, J=25.0 Hz, 1H), 7.29 (s, 1H), 7.27 (s, 2H), 6.21 (d, J=113.4 Hz, 2H), 4.79-4.46 (m, 2H), 4.42-4.24 (m, 2H), 3.85 (d, J=20.8 Hz, 1H), 3.52-3.38 (m, 1H), 3.20-3.10 (m, 1H), 2.98 (d, J=30.6 Hz, 3H), 1.90 (s, 1H), 1.80-1.63 (m, 1H).

### Example 115

### (2R,3R,3aS,6S,6aR)-6-((2-amino-3-(difluoromethyl)quinolin-7-yl)oxy)-2-(4-methyl-7H-pyrrolo[2,3-d]pyrimidin-7-yl)hexahydro-3aH-cyclopenta[b]furan-3,3a-diol

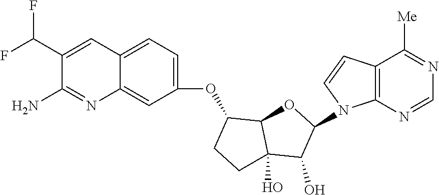 [SMILES: Cc1ncnc2c1ccn2[C@@H]1O[C@@H]2[C@@H](Oc3ccc4cc(C(F)F)c(N)nc4c3)CC[C@]2(O)[C@H]1O]

Step 1: To a stirred mixture of (3aR,4R,5aR,6R,8aR)-2,2-dimethyl-4-(4-methyl-7H-pyrrolo[2,3-d]pyrimidin-7-yl)hexahydrocyclopenta[2,3]furo[3,4-d][1,3]dioxol-6-ol (100 mg, 0.302 mmol) in DCM (1.5 mL) and pyridine (0.3 mL) was added trifluoromethanesulfonic anhydride (128 mg, 0.453 mmol) at 0° C. under an argon atmosphere. The resulting mixture was stirred for 1 h at 0° C. The reaction mixture was quenched by aqueous saturated NaHCO₃ (10 mL), extracted with EtOAc (25 mL×3). The combined organic layers were dried over anhydrous Na₂SO₄, filtered, and the filtrate was concentrated under reduced pressure. The residue was purified by column chromatography on silica (0-50% EtOAc/PE) to afford (3aR,4R,5aR,6R,8aR)-2,2-dimethyl-4-(4-methyl-7H-pyrrolo[2,3-d]pyrimidin-7-yl)hexahydrocyclopenta[2,3]furo[3,4-d][1,3]dioxol-6-yl trifluoromethanesulfonate as an oil. MS: 464 (M+1). ¹H-NMR (300 MHz, DMSO-d₆) δ 8.76 (s, 1H), 7.85 (d, J=3.6 Hz, 1H), 6.90 (d, J=3.9 Hz, 1H), 6.30 (d, J=5.1 Hz, 1H), 5.34 (d, J=4.8 Hz, 1H), 4.91 (d, J=3.6 Hz, 1H), 4.54-4.52 (m, 1H), 2.71 (s, 3H), 2.65-2.50 (m, 1H), 2.45-2.13 (m, 3H), 1.59 (s, 3H), 1.40 (s, 3H).

Step 2: To a stirred mixture of (3aR,4R,5aR,6R,8aR)-2,2-dimethyl-4-(4-methyl-7H-pyrrolo[2,3-d]pyrimidin-7-yl)hexahydrocyclopenta[2,3]furo[3,4-d][1,3]dioxol-6-yl trifluoromethanesulfonate (100 mg, 0.216 mmol), 3-(difluoromethyl)-2-((4-methoxybenzyl)amino)quinolin-7-ol (71.3 mg, 0.216 mmol) in NMP (3 mL) was added Cs₂CO₃ (211 mg, 0.647 mmol) at 25° C. under an argon atmosphere. The resulting mixture was stirred at 25° C. for 2 h. The reaction mixture was diluted with water (50 mL), and extracted with DCM (50 mL×3). The combined organic layers were dried over anhydrous Na₂SO₄, filtered, and the filtrate was concentrated under reduced pressure. The residue was purified by reverse phase column chromatography (ACN/water with 5 mM NH₄HCO₃ modifier) to afford 3-(difluoromethyl)-7-(((3aR,4R,5aR,6S,8aR)-2,2-dimethyl-4-(4-methyl-7H-pyrrolo[2,3-d]pyrimidin-7-yl)hexahydrocyclopenta[2,3]furo[3,4-d][1,3]dioxol-6-yl)oxy)-N-(4-methoxybenzyl)quinolin-2-amine as a solid. MS: 644 (M+1). ¹H-NMR (300 MHz, DMSO-d₆) δ 8.73 (s, 1H), 8.16 (s, 1H), 7.88 (d, J=3.6 Hz, 1H), 7.72 (d, J=8.7 Hz, 1H), 7.38-7.19 (m, 2H), 7.09-6.83 (m, 7H), 6.32 (d, J=4.8 Hz, 1H), 5.35 (d, J=5.1 Hz, 1H), 4.85-4.84 (m, 1H), 4.69-4.61 (m, 2H), 4.41-4.39 (m, 1H), 3.72 (s, 3H), 2.70 (s, 3H), 2.60-2.16 (m, 4H), 1.55 (s, 3H), 1.41 (s, 3H).

Step 3: To 3-(difluoromethyl)-7-(((3aR,4R,5aR,6S,8aR)-2,2-dimethyl-4-(4-methyl-7H-pyrrolo[2,3-d]pyrimidin-7-yl)hexahydrocyclopenta[2,3]furo[3,4-d][1,3]dioxol-6-yl)oxy)-N-(4-methoxybenzyl)quinolin-2-amine (100 mg, 0.155 mmol) was added TFA (5 mL) at 25° C. The resulting mixture was stirred for 1.5 h at 50° C. The reaction mixture was azeotroped with toluene five times (20 mL) to remove TFA. The residue was added to water (5 mL) and TFA (5 mL) at 25° C. The mixture was stirred for 16 h at 25° C. The mixture was azeotroped with toluene five times (20 mL) to remove TFA and water. The residue was purified by reverse phase column chromatography (ACN/water with 5 mM NH₄HCO₃ modifier) to afford (2R,3R,3aS,6S,6aR)-6-((2-amino-3-(difluoromethyl)quinolin-7-yl)oxy)-2-(4-methyl-7H-pyrrolo[2,3-d]pyrimidin-7-yl)hexahydro-2H-cyclopenta[b]furan-3,3a-diol as a solid. MS: 484 (M+1). ¹H-NMR (300 MHz, DMSO-d₆) δ 8.70 (s, 1H), 8.14 (s, 1H), 7.96 (d, J=3.6 Hz, 1H), 7.70 (d, J=8.7 Hz, 1H), 7.27-6.84 (m, 4H), 6.45 (br s, 2H), 6.16 (d, J=8.4 Hz, 1H), 5.47 (d, J=7.2 Hz, 1H), 5.41 (s, 1H), 4.66 (d, J=5.4 Hz, 1H), 4.51-4.46 (m, 1H), 4.14 (s, 1H), 2.72 (s, 3H), 2.54-2.50 (m, 1H), 2.10-1.96 (m, 3H).

### Example 116

### (1S,2R,3R,5R)-3-(2-(2-amino-3-fluoroquinolin-7- yl)ethyl)-5-(4-amino-7H-pyrrolo[2,3-d]pyrimidin-7-yl)-3-methylcyclopentane-1,2-diol

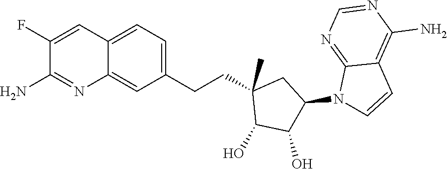 [SMILES: C[C@@]1(CCc2ccc3cc(F)c(N)nc3c2)C[C@@H](n2ccc3c(N)ncnc32)[C@H](O)[C@@H]1O]

Step 1: Under argon protection, to a mixture of ((3aR,4R,6R,6aS)-6-(4-chloro-7H-pyrrolo[2,3-d]pyrimidin-7-yl)-2,2-dimethyltetrahydro-3aH-cyclopenta[d][1,3]dioxol-4-yl)methanol (1.0 g, 3.09 mmol) in anhydrous DCM (20 mL) was added Dess-Martin Periodinane (2.62 g, 6.18 mmol) at 0° C. The resulting mixture was stirred at room temperature for 1.5 h. The reaction mixture was quenched with saturated aqueous Na₂S20₃ (20 mL) at 0° C. and extracted with DCM (2×60 mL). The combined organic layers were washed with saturated aqueous NaHCO₃ (60 mL) and brine (60 mL) sequentially, dried over anhydrous Na₂SO₄, filtered, and the filtrate was concentrated under reduced pressure. The residue was purified by column chromatography on silica (0-33% EtOAc/PE) to afford (3aR,4S,6R,6aS)-6-(4-chloro-7H-pyrrolo[2,3-d]pyrimidin-7-yl)-2,2-dimethyltetrahydro-3aH-cyclopenta[d][1,3]dioxole-4-carbaldehyde as an oil. MS: 322 (M+1). ¹H-NMR (400 MHz, DMSO-d₆) δ 9.75 (s, 1H), 8.65 (s, 1H), 7.92 (d, J=4.0 Hz, 1H), 6.73 (d, J=3.6 Hz, 1H), 5.22-5.16 (m, 1H), 5.11 (dd, J=6.8, 4.8 Hz, 1H), 4.97 (dd, J=7.2, 5.2 Hz, 1H), 3.19-3.14 (m, 1H), 2.54-2.46 (m, 2H), 1.51 (s, 3H), 1.27 (s, 3H).

Step 2: Compound (3aR,4S,6R,6aS)-6-(4-chloro-7H-pyrrolo[2,3-d]pyrimidin-7-yl)-2,2-dimethyl tetrahydro-3aH-cyclopenta[d][1,3]dioxole-4-carbaldehyde (1.9 g, 5.91 mmol) was dissolved in 1,4-dioxane (60 mL) at room temperature. Then aqueous formaldehyde (37 wt % in water, 0.701 mL, 7.09 mmol) and aqueous potassium carbonate (2M, 14.76 mL, 29.5 mmol) were added at room temperature. The resultant mixture was stirred at room temperature for 16 h. The reaction mixture was neutralized with aqueous AcOH (50 wt %) and then extracted with EtOAc (3×200 mL). The combined organic layers were washed with brine (50 mL), dried over anhydrous sodium sulfate, filtered, and the filtrate was concentrated under reduced pressure. The residue was dissolved in EtOH (60 mL) and treated with sodium tetrahydroborate (0.107 g, 2.83 mmol) at 0° C. The reaction mixture was stirred at 0° C. for 3 h. The mixture was then concentrated under reduced pressure. The resulting residue was diluted with water (50 mL). The pH of the mixture was adjusted to 7 with aqueous AcOH (50 wt %). Then the mixture was extracted with EtOAc (3×200 mL). The combined organic layers were washed with brine (50 mL), dried over anhydrous sodium sulfate, filtered, and the filtrate was concentrated under reduced pressure. The residue was purified by silica gel column chromatography (9:10:1 DCM/EtOAc/MeOH) to afford ((3aR,6R,6aS)-6-(4-chloro-7H-pyrrolo[2,3-d]pyrimidin-7-yl)-2,2-dimethyltetrahydro-3aH-cyclopenta[d][1,3]dioxole-4,4-diyl)dimethanol as a foam. MS: 354 (M+1). ¹H-NMR (400 MHz, DMSO-d₆) δ 8.67 (s, 1H), 8.01 (d, J=3.6 Hz, 1H), 6.74 (d, J=3.6 Hz, 1H), 5.27-5.20 (m, 1H), 4.95 (t, J=6.4 Hz, 1H), 4.89 (t, J=5.2 Hz, 1H), 4.63 (d, J=7.2 Hz, 1H), 4.50 (t, J=5.6 Hz, 1H), 3.60-3.49 (m, 3H), 3.44-3.40 (m, 1H), 2.26-2.23 (m, 1H), 2.06-1.92 (m, 1H), 1.48 (s, 3H), 1.22 (s, 3H).

Step 3: To a solution of ((3aR,6R,6aS)-6-(4-chloro-7H-pyrrolo[2,3-d]pyrimidin-7-yl)-2,2-dimethyltetrahydro-3aH-cyclopenta[d][1,3]dioxole-4,4-diyl)dimethanol (2.5 g, 7.07 mmol) in DCM (26 mL) were added triethylamine (2.95 mL, 21.20 mmol) and TBDPS-Cl (3.63 mL, 14.13 mmol) at 0° C. under argon. The reaction mixture was then stirred at room temperature for 16 h. The resulting solution was concentrated under reduced pressure. The residue was purified by silica gel column chromatography (0-30% EtOAc/PE) to afford ((3aR,4S,6R,6aS)-4-(((tert-butyldiphenylsilyl)oxy)methyl)-6-(4-chloro-7H-pyrrolo[2,3-d]pyrimidin-7-yl)-2,2-dimethyltetrahydro-3aH-cyclopenta[d][1,3]dioxol-4-yl)methanol as a foam. MS: 592 (M+1). ¹H-NMR (400 MHz, DMSO-d₆) δ 8.60 (s, 1H), 7.90 (d, J=3.6 Hz, 1H), 7.67-7.64 (m, 4H), 7.49-7.41 (m, 6H), 6.70 (d, J=3.6 Hz, 1H), 5.24-5.17 (m, 1H), 5.01 (t, J=6.8 Hz, 1H), 4.73 (d, J=7.2 Hz, 1H), 4.60 (br s, 1H), 3.77 (d, J=10.0 Hz, 1H), 3.70 (d, J=11.2 Hz, 1H), 3.62-3.58 (m, 2H), 2.29-2.24 (m, 1H), 2.20-2.14 (m, 1H), 1.47 (s, 3H), 1.22 (s, 3H), 1.05 (s, 9H). The column also afforded ((3aR,4R,6R,6aS)-4-(((tert-butyldiphenylsilyl)oxy)methyl)-6-(4-chloro-7H-pyrrolo[2,3-d]pyrimidin-7-yl)-2,2-dimethyltetrahydro-3aH-cyclopenta[d][1,3]dioxol-4-yl)methanol as a foam. MS: 592 (M+1). ¹H NMR (400 MHz, DMSO-d₆) δ 8.49 (s, 1H), 7.80 (d, J=3.6 Hz, 1H), 7.53-7.51 (m, 4H), 7.29-7.24 (m, 6H), 6.56 (d, J=3.6 Hz, 1H), 5.04-4.99 (m, 1H), 4.87 (br s, 1H), 4.77 (t, J=6.4 Hz, 1H), 4.44 (d, J=6.4 Hz, 1H), 3.62 (d, J=10.0 Hz, 1H), 3.51 (d, J=10.4 Hz, 1H), 3.46 (d, J=10.4 Hz, 1H), 3.38 (d, J=10.4 Hz, 1H), 2.13-2.10 (m, 1H), 1.93-1.87 (m, 1H), 1.13 (s, 3H), 1.00 (s, 3H), 0.81 (s, 9H).

Step 4: To a stirred solution of ((3aR,4R,6R,6aS)-4-(((tert-butyldiphenylsilyl)oxy)methyl)-6-(4-chloro-7H-pyrrolo[2,3-d]pyrimidin-7-yl)-2,2-dimethyltetrahydro-3aH-cyclopenta[d][1,3]dioxol-4-yl)methanol (1.7 g, 2.87 mmol) in toluene (50 mL) were added triphenylphosphine (3.01 g, 11.5 mmol), 1H-imidazole (782 mg, 11.5 mmol), and diiodine (1.46 g, 5.74 mmol) at room temperature under argon. Then the mixture was stirred at 120° C. for 3 h. The solvent was removed under reduced pressure, and the residue was purified by silica gel column chromatography (0-40% EtOAc/PE) to afford 7-((3aS,4R,6R,6aR)-6-(((tert-butyldiphenylsilyl)oxy)methyl)-6-(iodomethyl)-2,2-dimethyltetrahydro-3aH-cyclopenta[d][1,3]dioxol-4-yl)-4-chloro-7H-pyrrolo[2,3-d]pyrimidine as an oil. MS: 702 (M+1). ¹H NMR (400 MHz, Methanol-d₄) δ 8.63 (s, 1H), 7.82-7.78 (m, 4H), 7.70 (d, J=3.6 Hz, 1H), 7.50-7.45 (m, 6H), 6.74 (d, J=3.6 Hz, 1H), 5.16-5.10 (m, 1H), 5.08-5.04 (m, 1H), 4.68 (d, J=7.2 Hz, 1H), 3.96 (d, J=11.2 Hz, 1H), 3.75 (t, J=10.0 Hz, 2H), 3.63 (d, J=11.2 Hz, 1H), 2.63-2.57 (m, 1H), 2.40-2.23 (m, 1H), 1.33 (s, 3H), 1.27 (s, 3H), 1.09 (s, 9H).

Step 5: To a stirred solution of 7-((3aS,4R,6R,6aR)-6-(((tert-butyldiphenylsilyl)oxy)methyl)-6-(iodomethyl)-2,2-dimethyltetrahydro-3aH-cyclopenta[d][1,3]dioxol-4-yl)-4-chloro-7H-pyrrolo[2,3-d]pyrimidine (1.6 g, 2.28 mmol) in 1,4-dioxane (40 mL) was added sodium phenolate (661 mg, 5.70 mmol) at room temperature under argon. The reaction mixture was stirred at 80° C. overnight. The reaction mixture was then directly purified by silica gel column chromatography (0-40% EtOAc/PE) to afford 7-((3aS,4R,6R,6aR)-6-(((tert-butyldiphenylsilyl)oxy)methyl)-6-(iodomethyl)-2,2-dimethyltetrahydro-3aH-cyclopenta[d][1,3]dioxol-4-yl)-4-phenoxy-7H-pyrrolo[2,3-d]pyrimidine as an oil. MS: 760 (M+1). ¹H NMR (400 MHz, DMSO-d₆) δ 8.43 (s, 1H), 7.78-7.72 (m, 6H), 7.51-7.47 (m, 10H), 6.62 (d, J=3.6 Hz, 1H), 5.22-5.16 (m, 1H), 5.09 (t, J=6.4 Hz, 1H), 4.61 (d, J=6.8 Hz, 1H), 3.85 (d, J=11.2 Hz, 1H), 3.77-3.70 (m, 2H), 3.63 (d, J=11.2 Hz, 1H), 2.56-2.54 (m, 1H), 2.31-2.28 (m, 1H), 1.24 (s, 3H), 1.19 (s, 3H), 1.03 (s, 9H).

Step 6: To a stirred solution of 7-((3aS,4R,6R,6aR)-6-(((tert-butyldiphenylsilyl)oxy)methyl)-6-(iodomethyl)-2,2-dimethyltetrahydro-3aH-cyclopenta[d][1,3]dioxol-4-yl)-4-phenoxy-7H-pyrrolo[2,3-d]pyrimidine (1.6 g, 2.11 mmol) in EtOAc (20 mL) and ethanol (20 mL) was added dihydroxypalladium on carbon (20 wt %, 1600 mg, 2.28 mmol) at room temperature under nitrogen. The suspension was degassed under vacuum and purged with H₂ several times, then the mixture was stirred under 1-2 atm of H₂ at 25° C. for 30 min. The mixture was then filtered through a Celite pad, and the filtrate was concentrated under reduced pressure to afford 7-((3aS,4R,6S,6aR)-6-(((tert-butyldiphenylsilyl)oxy)methyl)-2,2,6-trimethyltetrahydro-3aH-cyclopenta[d][1,3]dioxol-4-yl)-4-phenoxy-7H-pyrrolo[2,3-d]pyrimidine as an oil. MS: 634 (M+1). ¹H-NMR (400 MHz, DMSO-d₆) δ 8.41 (s, 1H), 7.79 (d, J=2.7 Hz, 1H), 7.72-7.68 (m, 4H), 7.51-7.46 (m, 8H), 7.34-7.27 (m, 3H), 6.58 (d, J=2.7 Hz, 1H), 5.22-5.11 (m, 1H), 5.06 (t, J=4.8 Hz, 1H), 4.52 (d, J=5.1 Hz, 1H), 3.71 (d, J=7.8 Hz, 1H), 3.61 (d, J=7.8 Hz, 1H), 2.50-2.44 (m, 1H), 2.15-2.02 (m, 1H), 1.30 (s, 3H), 1.29 (s, 3H), 1.20 (s, 3H), 1.03 (s, 9H).

Step 7: To a stirred solution of 7-((3aS,4R,6S,6aR)-6-(((tert-butyldiphenylsilyl)oxy)methyl)-2,2,6-trimethyltetrahydro-3aH-cyclopenta[d][1,3]dioxol-4-yl)-4-phenoxy-7H-pyrrolo[2,3-d]pyrimidine (1.1 g, 1.74 mmol) in THF (10 mL) was added tetrabutylammonium fluoride (1M in THF) (3.47 mL, 3.47 mmol) at room temperature under argon. The reaction was stirred at 45° C. overnight. After completion, the solvent was removed under reduced pressure, and the residue was purified by silica gel column chromatography (0-40% EtOAc/PE) to afford ((3aR,4S,6R,6aS)-2,2,4-trimethyl-6-(4-phenoxy-7H-pyrrolo[2,3-d]pyrimidin-7-yl)tetrahydro-3aH-cyclopenta[d][1,3]dioxol-4-yl)methanol as a solid. MS: 396 (M+1). ¹H-NMR (300 MHz, DMSO-d₆) δ 8.37 (s, 1H), 7.80 (d, J=3.9 Hz, 1H), 7.51-7.46 (m, 2H), 7.34-7.26 (m, 3H), 6.56 (d, J=3.6 Hz, 1H), 5.24-5.18 (m, 1H), 5.02 (t, J=6.3 Hz, 1H), 4.57 (t, J=4.8 Hz, 1H), 4.49 (d, J=7.2 Hz, 1H), 3.55-3.51 (m, 1H), 3.42-3.37 (m, 1H), 2.42-2.35 (m, 1H), 2.05-1.97 (m, 1H), 1.47 (s, 3H), 1.23 (s, 3H), 1.16 (s, 3H).

Step 8: To a stirred solution of ((3aR,4S,6R,6aS)-2,2,4-trimethyl-6-(4-phenoxy-7H-pyrrolo[2,3-d]pyrimidin-7-yl)tetrahydro-3aH-cyclopenta[d][1,3]dioxol-4-yl)methanol (520 mg, 1.315 mmol) in DCM (10 mL) was added Dess-Martin Periodinane (837 mg, 1.97 mmol) at room temperature under argon. The reaction was stirred at this temperature for 30 min. The resultant mixture was quenched with saturated aqueous NaHCO₃ (1M, 20 mL), and the mixture was extracted with DCM (3×30 mL). The combined organic layers were washed with brine (3×20 mL), dried over anhydrous sodium sulfate, and filtered. The filtrate was then concentrated under reduced pressure, and the resulting crude (3aR,4R,6R,6aS)-2,2,4-trimethyl-6-(4-phenoxy-7H-pyrrolo[2,3-d]pyrimidin-7-yl)tetrahydro-3aH-cyclopenta[d][1,3]dioxole-4-carbaldehyde was used directly in the next step.

Step 9: To a stirred mixture of bromo(methyl)triphenylphosphorane (1.32 g, 3.68 mmol) in THF (20 mL) was added n-butyllithium (2.5M in hexane, 1.37 mL, 3.42 mmol) at −60° C. under argon. The reaction mixture was warmed to room temperature and kept for −30 minutes. Then crude (3aR,4R,6R,6aS)-2,2,4-trimethyl-6-(4-phenoxy-7H-pyrrolo[2,3-d]pyrimidin-7-yl)tetrahydro-3aH-cyclopenta[d][1,3]dioxole-4-carbaldehyde (1.315 mmol) in THF (5 mL) was added dropwise to the above solution at −40° C. under argon. The resultant mixture was stirred at room temperature for 3 h. The reaction was quenched with saturated aqueous NH₄Cl (20 mL) at −40° C. and extracted with EtOAc (3×50 mL). The combined organic fractions were washed with brine (3×20 mL), dried over anhydrous sodium sulfate, and filtered. The filtrate was concentrated under reduced pressure, and the resulting residue was purified by silica gel column chromatography (0-40% EtOAc/PE) to afford 4-phenoxy-7-((3aS,4R,6S,6aR)-2,2,6-trimethyl-6-vinyltetrahydro-3aH-cyclopenta[d][1,3]dioxol-4-yl)-7H-pyrrolo[2,3-d]pyrimidine as a solid. MS: 392 (M+1). ¹H-NMR (400 MHz, DMSO-d6) δ 8.36 (s, 1H), 7.81 (d, J=3.6 Hz, 1H), 7.50-7.46 (m, 2H), 7.32-7.25 (m, 3H), 6.55 (d, J=3.6 Hz, 1H), 6.20-6.13 (m, 1H), 5.20-5.12 (m, 3H), 5.04 (dd, J=5.2, 7.2 Hz, 1H), 4.50 (d, J=7.2 Hz, 1H), 2.39-2.32 (m, 2H), 1.47 (s, 3H), 1.23 (s, 3H), 1.22 (s, 3H).

Step 10: 4-phenoxy-7-((3aS,4R,6S,6aR)-2,2,6-trimethyl-6-vinyltetrahydro-3aH-cyclopenta[d][1,3]dioxol-4-yl)-7H-pyrrolo[2,3-d]pyrimidine (272 mg, 0.695 mmol) was dissolved in 1,4-dioxane (15 mL) in a sealed tube, and then the mixture was treated with concentrated NH₃.H₂O (28 wt %, 15 mL). The resultant mixture was heated at 120° C. for 16 h. The volatiles were removed under reduced pressure, and the residue was purified by prep TLC (1:1 PE:EtOAc) to afford 7-((3aS,4R,6S,6aR)-2,2,6-trimethyl-6-vinyltetrahydro-3aH-cyclopenta[d][1,3]dioxol-4-yl)-7H-pyrrolo[2,3-d]pyrimidin-4-amine as a solid. MS: 315 (M+1). ¹H-NMR (400 MHz, DMSO-d₆) δ 8.06 (s, 1H), 7.38 (d, J=3.6 Hz, 1H), 6.97 (br s, 2H), 6.57 (d, J=3.6 Hz, 1H), 6.18-6.11 (m, 1H), 5.17-5.08 (m, 2H), 5.08-5.04 (m, 1H), 4.98 (dd, J=4.8, 7.2 Hz, 1H), 4.47 (d, J=6.8 Hz, 1H), 2.33-2.20 (m, 2H), 1.45 (s, 3H), 1.22 (s, 3H), 1.20 (s, 3H).

Step 11: To a 20 mL microwave tube charged with 7-((3aS,4R,6S,6aR)-2,2,6-trimethyl-6-vinyltetrahydro-3aH-cyclopenta[d][1,3]dioxol-4-yl)-7H-pyrrolo[2,3-d]pyrimidin-4-amine (174 mg, 0.553 mmol) was added 9-borabicyclo[3.3.1]nonane (0.5M in THF, 4.43 mL, 2.21 mmol) at room temperature under argon. The reaction mixture was heated at 60° C. for 1 h. The mixture was cooled to 0° C. after completion. To this mixture was added a solution of potassium phosphate (587 mg, 2.77 mmol) in water (1.3 mL) at 0° C. After stirring at room temperature for 30 min, Pd(dppf)Cl₂ (41 mg, 0.055 mmol) and a solution of 7-bromo-3-fluoroquinolin-2-amine (147 mg, 0.608 mmol) in THF (4 mL) were added. The final mixture was irradiated with microwave irradiation at 80° C. for 2 h. The reaction mixture was then diluted with water (20 mL) and the mixture was extracted with EtOAc (3×50 mL). The combined organic layers were washed with brine (3×20 mL), dried over anhydrous sodium sulfate, and filtered. The filtrate was concentrated under reduced pressure, and the residue was purified by prep-TLC (20:1 EtOAc:MeOH) to afford 7-(2-((3aR,4R,6R,6aS)-6-(4-amino-7H-pyrrolo[2,3-d]pyrimidin-7-yl)-2,2,4-trimethyltetrahydro-3aH-cyclopenta[d][1,3]dioxol-4-yl)ethyl)-3-fluoroquinolin-2-amine as a solid. MS: 477 (M+1). ¹H-NMR (400 MHz, DMSO-d₆) δ 8.06 (s, 1H), 7.78 (d, J=11.6 Hz, 1H), 7.60 (d, J=8.4 Hz, 1H), 7.38-7.36 (m, 2H), 7.14 (d, J=8.4 Hz, 1H), 6.97 (br s, 2H), 6.71 (br s, 2H), 6.57 (d, J=3.6 Hz, 1H), 5.05-5.02 (m, 2H), 4.44 (d, J=4.0 Hz, 1H), 2.77-2.70 (m, 2H), 2.27-2.22 (m, 1H), 2.15-2.09 (m, 1H), 1.88-1.73 (m, 2H), 1.46 (s, 3H), 1.25 (s, 3H), 1.21 (s, 3H).

Step 12: 7-(2-((3aR,4R,6R,6aS)-6-(4-amino-7H-pyrrolo[2,3-d]pyrimidin-7-yl)-2,2,4-trimethyltetrahydro-4H-cyclopenta[d][1,3]dioxol-4-yl)ethyl)-3-fluoroquinolin-2-amine (129.4 mg, 0.272 mmol) in TFA (2.72 mL) and water (2.72 mL) was stirred for overnight at 40° C. The reaction mixture was then concentrated under reduced pressure, and the residue was purified by mass-triggered HPLC (ACN/water with 0.1% TFA modifier) to afford (1S,2R,3R,5R)-3-(2-(2-amino-3-fluoroquinolin-7-yl)ethyl)-5-(4-amino-7H-pyrrolo[2,3-d]pyrimidin-7-yl)-3-methylcyclopentane-1,2-diol, TFA salt, as an oil. MS: 437 (M+1). ¹H NMR (DMSO-d₆) δ: 8.36 (s, 1H), 8.10 (d, J=11.2 Hz, 2H), 7.79-7.64 (m, 2H), 7.44 (s, 1H), 7.29 (d, J=8.1 Hz, 1H), 7.24 (s, 1H), 7.14 (s, 1H), 7.04 (s, 1H), 6.96 (d, J=3.6 Hz, 1H), 5.11-5.03 (m, 1H), 5.02-4.79 (m, 1H), 4.65-4.53 (m, 1H), 3.66 (d, J=4.2 Hz, 1H), 2.80 (td, J=12.7, 4.3 Hz, 1H), 2.67 (td, J=12.6, 4.3 Hz, 1H), 2.15 (dd, J=13.5, 10.4 Hz, 1H), 1.88 (td, J=12.9, 4.6 Hz, 1H), 1.67 (td, J=12.9, 4.6 Hz, 1H), 1.60 (dd, J=13.5, 8.9 Hz, 1H), 1.27 (s, 3H).

### Example 117

### (1R,2S,3R,5S)-5-(2-(2-amino-3-bromoquinolin-7-yl)ethyl)-3-(4-amino-7H-pyrrolo[2,3-d]pyrimidin-7-yl)-1,5-dimethylcyclopentane-1,2-diol

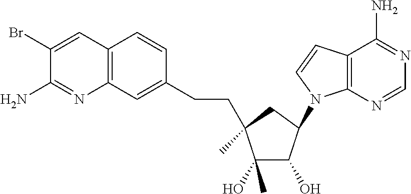 [SMILES: C[C@@]1(CCc2ccc3cc(Br)c(N)nc3c2)C[C@@H](n2ccc3c(N)ncnc32)[C@H](O)[C@]1(C)O]

Step 1: (1R,4S)-tert-butyl 5-methyl-3-oxo-2-azabicyclo[2.2.1]hept-5-ene-2-carboxylate (1 g, 4.48 mmol) was dissolved in 4 M HCl/MeOH (10 mL), and the reaction mixture was heated to reflux and stirred at this temperature for 2 h. The solvent was then removed under reduced pressure to give crude (1S,4R)-methyl 4-amino-2-methylcyclopent-2-enecarboxylate hydrochloride which was used directly in the next step.

Step 2: To a stirred solution of (1S,4R)-methyl 4-amino-2-methylcyclopent-2-enecarboxylate hydrochloride (859 mg, 4.48 mmol) in 5:1 Acetone: H₂O (12 mL) were added sodium bicarbonate (753 mg, 8.96 mmol) and di-tert-butyl dicarbonate (1076 mg, 4.93 mmol). The reaction mixture was stirred at 25° C. for 2 h. The reaction mixture was diluted with 100 mL of water and extracted with 100 mL EtOAc. The organic phase was then washed with 100 mL of brine, dried over anhydrous sodium sulfate, filtered, and the filtrate was concentrated under reduced pressure. The residue was purified by silica gel chromatography (3:1 PE/EtOAc) to give (1S,4R)-methyl 4-((tert-butoxycarbonyl)amino)-2-methylcyclopent-2-enecarboxylate as an oil. ¹H NMR (400 MHz, Chloroform-d) δ 5.58-5.52 (m, 1H), 5.04 (s, 1H), 4.72 (s, 1H), 3.75 (s, 3H), 3.32-3.24 (m, 1H), 2.53 (dt, J=13.9, 8.5 Hz, 1H), 1.90 (dt, J=13.9, 3.2 Hz, 1H), 1.77 (q, J=1.3 Hz, 3H), 1.46 (s, 9H).

Step 3: To a stirred solution of (1S,4R)-methyl 4-((tert-butoxycarbonyl)amino)-2-methylcyclopent-2-enecarboxylate (4.3 g, 16.84 mmol) in THF (80 mL) was added 1M lithium bis(trimethylsilyl)amide in THF (38.7 mL, 38.7 mmol, 1M) at −78° C., and the reaction mixture was stirred at −78° C. for 0.5 h. Then iodomethane (2.63 g, 18.53 mmol) was added dropwise at −78° C. and the resulting mixture was stirred at −20° C. for 2 h. The reaction was quenched with saturated aqueous NH₄Cl (100 mL) and extracted with EtOAc (120 mL×3). The combined organic layers were dried over anhydrous sodium sulfate, filtered, and the filtrate was concentrated under reduced pressure. The residue was purified by silica gel column chromatography (20-25% EtOAc/PE) to afford (1S,4R)-methyl 4-((tert-butoxycarbonyl)amino)-1,2-dimethylcyclopent-2-enecarboxylate as an oil. ¹H NMR (300 MHz, Chloroform-d) δ 5.49 (p, J=1.5 Hz, 1H), 5.03 (s, 1H), 4.69 (s, 1H), 3.72 (s, 3H), 2.21-2.15 (m, 2H), 1.69 (t, J=1.5 Hz, 3H), 1.46 (s, 9H), 1.31 (s, 3H).

Step 4: To a stirred solution of (1S,4R)-methyl 4-((tert-butoxycarbonyl)amino)-1,2-dimethylcyclopent-2-enecarboxylate (3.1 g, 11.5 mmol) in THF (50 mL) was added lithium borohydride (2M in THF, 11.5 mL, 23.02 mmol) dropwise at 0° C. Then the reaction mixture was stirred at room temperature for 16 h. The mixture was quenched with water (100 mL) and extracted with EtOAc (100 mL×3). The combined organic layers were dried over anhydrous sodium sulfate, filtered, and the filtrate was concentrated under reduced pressure. The residue was purified by silica gel column chromatography (18-22% EtOAc/PE) to afford tert-butyl ((1R,4S)-4-(hydroxymethyl)-3,4-dimethylcyclopent-2-en-1-yl)carbamate as a solid. ¹H NMR (400 MHz, Chloroform-d) δ 5.47 (s, 1H), 4.58 (d, J=8.7 Hz, 1H), 3.54 (d, J=10.5 Hz, 1H), 3.31 (d, J=10.5 Hz, 1H), 2.13 (dd, J=13.8, 8.7 Hz, 1H), 1.76 (dd, J=13.8, 3.0 Hz, 1H), 1.66 (d, J=1.5 Hz, 3H), 1.45 (s, 9H), 1.00 (s, 3H).

Step 5: To a stirred solution of tert-butyl ((1R,4S)-4-(hydroxymethyl)-3,4-dimethylcyclopent-2-en-1-yl)carbamate (550 mg, 2.279 mmol) in 1:1 tBuOH:H₂O (6 mL) was added 4-methylmorpholine 4-oxide (534 mg, 4.56 mmol) at room temperature. Then the mixture was cooled to 0° C. and 4% osmium(VIII) oxide in water (1.88 g, 0.296 mmol) was added at 0° C. The reaction mixture was stirred at 0° C. for 16 h. The reaction was quenched with 50 mL saturated aqueous sodium thiosulfate and extracted with 50 mL EtOAc. The organic layer was washed with 50 mL water and 50 mL brine, dried over anhydrous sodium sulfate, filtered, and the filtrate was concentrated under reduced pressure. The residue was purified by silica gel chromatography (1:1 PE/EtOAc) to give tert-butyl ((1R,2S,3R,4R)-2,3-dihydroxy-4-(hydroxymethyl)-3,4-dimethylcyclopentyl)carbamate as a solid. Then tert-butyl ((1R,2S,3R,4R)-2,3-dihydroxy-4-(hydroxymethyl)-3,4-dimethylcyclopentyl)carbamate (530 mg, 1.93 mmol) was dissolved in HCl in MeOH (4M, 10 mL), and the reaction mixture was stirred at 25° C. for 2 h. The solvent was removed under reduced pressure to give crude product which was used directly in the next step. Then to a stirred solution of (1R,2S,3R,5R)-3-amino-5-(hydroxymethyl)-1,5-dimethylcyclopentane-1,2-diol hydrochloride (402 mg, 1.9 mmol) in i-PrOH (6 mL) were added DIEA (0.664 mL, 3.80 mmol) and 4,6-dichloro-5-(2,2-diethoxyethyl)pyrimidine (554 mg, 2.09 mmol) at room temperature. The reaction mixture was heated to 100° C. and stirred at this temperature for 8 h. The solvent was then removed under reduced pressure, and the resulting crude residue was purified by silica gel column chromatography (eluting with EtOAc) to give (1R,2S,3R,5R)-3-((6-chloro-5-(2,2-diethoxyethyl)pyrimidin-4-yl)amino)-5-(hydroxymethyl)-1,5-dimethylcyclopentane-1,2-diol as an oil. MS: 404 (M+1).

Step 6: To a stirred solution of (1R,2S,3R,5R)-3-((6-chloro-5-(2,2-diethoxyethyl)pyrimidin-4-yl)amino)-5-(hydroxymethyl)-1,5-dimethylcyclopentane-1,2-diol (230 mg, 0.569 mmol) in dioxane (3 mL) was added HCl in water (4M, 0.285 mL, 1.139 mmol) at room temperature. The resulting mixture was warmed to 50° C. and stirred at this temperature for 15 min. The reaction mixture was quenched with 20 mL of saturated aqueous sodium bicarbonate and extracted with EtOAc (3×20 mL). The organic layers were combined, dried over anhydrous sodium sulfate, filtered, and the filtrate was concentrated under reduced pressure to give (1R,2S,3R,5R)-3-(4-chloro-7H-pyrrolo[2,3-d]pyrimidin-7-yl)-5-(hydroxymethyl)-1,5-dimethylcyclopentane-1,2-diol as a solid. MS: 312 (M+1).

Step 7: To a stirred solution of (1R,2S,3R,5R)-3-(4-chloro-7H-pyrrolo[2,3-d]pyrimidin-7-yl)-5-(hydroxymethyl)-1,5-dimethylcyclopentane-1,2-diol (540 mg, 1.73 mmol) in acetone (15 mL) were added 4-methylbenzenesulfonic acid (30 mg, 0.173 mmol) and 2,2-dimethoxypropane (1.80 gg, 17.3 mmol) at 0° C. The reaction mixture was stirred at 25° C. for 16 h. The mixture was concentrated under reduced pressure, and the residue was purified by silica gel column chromatography (54% EtOAc/PE) to afford ((3aR,4R,6R,6aS)-6-(4-chloro-7H-pyrrolo[2,3-d]pyrimidin-7-yl)-2,2,3a,4-tetramethyltetrahydro-3aH-cyclopenta[d][1,3]dioxol-4-yl)methanol as an oil. MS: 352 (M+1).

Step 8: To a stirred solution of oxalyl chloride (0.30 mL, 3.41 mmol) in DCM (8 mL) was added dropwise DMSO (0.484 mL, 6.82 mmol) at −78° C., and the resulting mixture was stirred at −78° C. for 0.5 h. Then ((3aR,4R,6R,6aS)-6-(4-chloro-7H-pyrrolo[2,3-d]pyrimidin-7-yl)-2,2,3a,4-tetramethyltetrahydro-3aH-cyclopenta[d][1,3]dioxol-4-yl)methanol (400 mg, 1.137 mmol) in DCM (3 mL) was added dropwise, and the reaction mixture kept stirring at −78° C. for another 0.5 h. Then TEA (1.585 mL, 11.37 mmol) was added at −78° C., and the reaction mixture was warmed to room temperature and stirred at this temperature for 1 h. Then 100 mL of saturated aqueous ammonium chloride was added, and the mixture was extracted with EtOAc (2×100 mL). The combined organic layers were dried over anhydrous sodium sulfate, filtered, and the filtrate was concentrated under reduced pressure to give crude (3aR,4S,6R,6aS)-6-(4-chloro-7H-pyrrolo[2,3d]pyrimidin-7-yl)-2,2,3a,4-tetramethyltetrahydro-3aH-cyclopenta[d][1,3]dioxole-4-carbaldehyde, which was used directly in the next step.

Step 9: To a stirred solution of methyltriphenylphosphonium bromide (1.14 g, 3.18 mmol) in THF (8 mL) was added n-butyllithium (2.5M in hexane, 0.455 mL, 1.14 mmol) dropwise at −20° C. The resulting mixture was warmed to room temperature and stirred for 1 h. Then a solution of (3aR,4S,6R,6aS)-6-(4-chloro-7H-pyrrolo[2,3-d]pyrimidin-7-yl)-2,2,3a,4-tetramethyltetrahydro-3aH-cyclopenta[d][1,3]dioxole-4-carbaldehyde (398 mg, 1.14 mmol) in THF (3 mL) was added dropwise at −20° C. The resulting mixture was then warmed to 25° C. and stirred for 2 h. The mixture was diluted with 100 mL of EtOAc and washed with 100 mL of water and 100 mL of brine. The organic layer was dried over anhydrous sodium sulfate, filtered, and the filtrate was concentrated under reduced pressure. The residue was purified by silica gel column chromatography (3:1 PE/EtOAc) to give 4-chloro-7-((3aS,4R,6R,6aR)-2,2,6,6a-tetramethyl-6-vinyltetrahydro-3aH-cyclopenta[d][1,3]dioxol-4-yl)-7H-pyrrolo[2,3-d]pyrimidine as an oil. MS: 348 (M+1).

Step 10: To a 25 mL sealed tube containing a solution of 4-chloro-7-((3aS,4R,6R,6aR)-2,2,6,6a-tetramethyl-6-vinyltetrahydro-3aH-cyclopenta[d][1,3]dioxol-4-yl)-7H-pyrrolo[2,3-d]pyrimidine (250 mg, 0.719 mmol) in dioxane (5 mL) was added 28 wt % NH₃ in H₂O (25 mL). The resulting mixture was heated to 90° C. and stirred for 16 h. The solvent was removed under reduced pressure to afford crude 7-((3aS,4R,6R,6aR)-2,2,6,6a-tetramethyl-6-vinyltetrahydro-3aH-cyclopenta[d][1,3]dioxol-4-yl)-7H-pyrrolo[2,3-d]pyrimidin-4-amine which was used directly in the next step.

Step 11: A 10 mL round bottom flask was charged with 7-((3aS,4R,6R,6aR)-2,2,6,6a-tetramethyl-6-vinyltetrahydro-3aH-cyclopenta[d][1,3]dioxol-4-yl)-7H-pyrrolo[2,3-d]pyrimidin-4-amine (70 mg, 0.213 mmol) and 9-BBN (0.5M in THF, 2.13 ml, 1.07 mmol) at room temperature under an argon atmosphere. The resulting mixture was heated to 50° C. and stirred for 1 h. To this crude reaction mixture was added a solution of potassium phosphate tribasic (226 mg, 1.07 mmol) in Water (0.2 mL) at 0° C., and the resulting mixture was heated to 50° C. and stirred for 0.5 h. Then a solution of 3-bromo-7-iodo-N-(4-methoxybenzyl)quinolin-2-amine (110 mg, 0.234 mmol) in THF (0.2 mL) and [1,1′-bis(diphenylphosphino)ferrocene]dichloropalladium (II) (15.59 mg, 0.021 mmol) were added at room temperature, and the resulting mixture was heated to 50° C. and stirred for 1 h. The mixture was diluted with 30 mL EtOAc and washed with 30 mL water and 30 mL brine. The combined organic layers were dried over anhydrous sodium sulfate, filtered, and the filtrate was concentrated under reduced pressure. The residue was purified by prep-TLC (10% MeOH/DCM) to afford 7-(2-((3aR,6R,6aS)-6-(4-amino-7H-pyrrolo[2,3-d]pyrimidin-7-yl)-2,2,3a,4-tetramethyltetrahydro-3aH-cyclopenta[d][1,3]dioxol-4-yl)ethyl)-3-bromo-N-(4-methoxybenzyl)quinolin-2-amine as a solid. MS: 671/673 (M+1/M+3).

Step 12: A 10 mL round bottom flask was charged with 7-(2-((3aR,6R,6aS)-6-(4-amino-7H-pyrrolo[2,3-d]pyrimidin-7-yl)-2,2,3a,4-tetramethyltetrahydro-3aH-cyclopenta[d][1,3]dioxol-4-yl)ethyl)-3-bromo-N-(4-methoxybenzyl)quinolin-2-amine (80 mg, 0.119 mmol) and TFA (2 mL) at room temperature under an argon atmosphere. The resulting mixture was then heated to 50° C. and stirred for 40 minutes. The solvent was removed under reduced pressure. The resulting crude material was purified by reverse phase column chromatography (ACN/water with 5 mM to afford (1R,2S,3R)-5-(2-(2-amino-3-bromoquinolin-7-yl)ethyl)-3-(4-amino-7H-pyrrolo[2,3-d]pyrimidin-7-yl)-1,5-dimethylcyclopentane-1,2-diol as a solid. MS: 511/513 (M+1/M+3). ¹H-NMR (300 MHz, DMSO-d₆) δ 8.33 (s, 1H), 8.04 (s, 1H), 7.58 (d, J=8.4 Hz, 1H), 7.36 (s, 1H), 7.18-7.13 (m, 2H), 6.89 (br s, 2H), 6.54-6.53 (m, 3H), 4.90 (d, J=6.9 Hz, 1H), 4.81 (q, J=9.0 Hz, 1H), 4.41-4.36 (m, 1H), 4.09 (s, 1H), 2.68-2.65 (m, 2H), 2.08-1.90 (m, 3H), 1.66-1.63 (m, 1H), 1.09 (s, 6H).

### Example 118

### (1R,2S,3S,4R)-1-(2-(2-amino-3-bromoquinolin-7-yl)ethyl)-4-(4-amino-7H-pyrrolo[2,3-d]pyrimidin-7-yl)-2-methylcyclopentane-1,2,3-triol

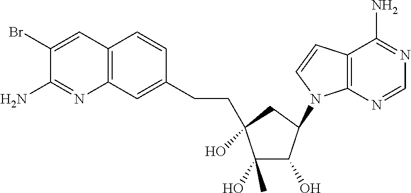 [SMILES: C[C@]1(O)[C@@H](O)[C@H](n2ccc3c(N)ncnc32)C[C@@]1(O)CCc1ccc2cc(Br)c(N)nc2c1]

Step 1: To a stirred solution of (1R,4S)-4-hydroxycyclopent-2-en-1-yl acetate (5.68 g, 40.0 mmol), 4-chloro-7H-pyrrolo[2,3-d]pyrimidine (9.20 g, 59.9 mmol), and triphenylphosphine (36.7 g, 140 mmol) in THF (80 mL) was added (E)-diisopropyl diazene-1,2-dicarboxylate (20.20 g, 100 mmol) under an argon atmosphere at 0° C. The resulting mixture was stirred at room temperature for 16 h. The solution was concentrated under reduced pressure, and the resulting residue was purified by column chromatography on silica (0-80% EtOAc/PE) to obtain (1R,4R)-4-(4-chloro-7H-pyrrolo[2,3-d]pyrimidin-7-yl)cyclopent-2-en-1-yl acetate as a solid. ¹H NMR (300 MHz, DMSO-d₆) δ 8.66 (s, 1H), 7.66 (d, J=3.7 Hz, 1H), 6.69 (d, J=3.6 Hz, 1H), 6.32-6.19 (m, 2H), 6.09 (ddt, J=7.4, 4.9, 1.9 Hz, 1H), 6.03-5.92 (m, 1H), 2.53-2.32 (m, 2H), 2.05 (s, 3H).

Step 2: To a stirred solution of (1R,4R)-4-(4-chloro-7H-pyrrolo[2,3-d]pyrimidin-7-yl)cyclopent-2-en-1-yl acetate (8.6 g, 31.0 mmol) in DCM (20 mL) was added ammonia in MeOH (200 mL, 7M, 1400 mmol), and the resulting mixture was stirred at room temperature for 16 h. The solution was concentrated under reduced pressure. The resulting residue was purified by column chromatography on silica (0-15% MeOH/DCM) to afford (1R,4R)-4-(4-chloro-7H-pyrrolo[2,3-d]pyrimidin-7-yl)cyclopent-2-enol as a solid. MS: 236 (M+1).

Step 3: To a solution of (1R,4R)-4-(4-chloro-7H-pyrrolo[2,3-d]pyrimidin-7-yl)cyclopent-2-enol (350 mg, 1.49 mmol) in anhydrous DCM (7 mL) was added Dess-Martin periodinane (945 mg, 2.23 mmol) at 0° C. under an argon atmosphere. The resulting mixture was stirred at 25° C. for 16 h. The reaction mixture was then cooled down to 0° C., quenched by saturated aqueous sodium bicarbonate (5 mL) and diluted with DCM (100 mL). The mixture solution was then filtered through Celite. The filtrate was concentrated under reduced pressure, and the resulting residue was purified by column chromatography on silica (20-35% EtOAc/PE) to afford (R)-4-(4-chloro-7H-pyrrolo[2,3-d]pyrimidin-7-yl)cyclopent-2-enone as a solid. MS: 234 (M+1).

Step 4: To a stirred solution of (R)-4-(4-chloro-7H-pyrrolo[2,3-d]pyrimidin-7-yl)cyclopent-2-enone (870 mg, 3.72 mmol) in DCM (3 mL) and pyridine (3 mL) was added a solution of diiodine (1.60 g, 6.33 mmol) in DCM (3 mL) and pyridine (3 mL) at 0° C. The resulting mixture was stirred at room temperature for 16 h. Then DCM (30 mL) and sodium thiosulfate solution (60 mL, 1M) were added to the solution, and the organic layer was concentrated under reduced pressure. The resulting residue was purified by column chromatography on silica (0-80% EtOAc/PE) to afford (R)-4-(4-chloro-7H-pyrrolo[2,3-d]pyrimidin-7-yl)-2-iodocyclopent-2-enone as a solid. MS: 360 (M+1).

Step 5: To a stirred solution of (R)-4-(4-chloro-7H-pyrrolo[2,3-d]pyrimidin-7-yl)-2-iodocyclopent-2-enone (2.9 g, 8.07 mmol) in N-Methyl-2-pyrrolidinone (25 mL) were added copper(I) iodide (0.614 g, 3.23 mmol), triphenylarsine (0.99 g, 3.23 mmol), Dichlorobis(benzonitrile)palladium(II) (1.24 g, 3.23 mmol), and tetramethylstannane (14.4 g, 81 mmol). The resulting mixture was then stirred at 80° C. under an argon atmosphere for 2 h. The mixture was cooled and purified by column chromatography on silica (0-70% EtOAc/PE). The isolated material was then dissolved in DCM (100 mL) and washed with water (60 mL×4). The organic layer was concentrated under reduced pressure, and the resulting residue was washed with 30:1 PE/EtOAc (40 mL) and then filtered to afford (R)-4-(4-chloro-7H-pyrrolo[2,3-d]pyrimidin-7-yl)-2-methylcyclopent-2-enone as a solid. MS: 248 (M+1).

Step 6: To a mixture of (R)-4-(4-chloro-7H-pyrrolo[2,3-d]pyrimidin-7-yl)-2-methylcyclopent-2-enone (900 mg, 3.63 mmol) and NMO (851 mg, 7.27 mmol) in THF (90 mL), water (9.0 mL), and acetone (9.0 mL) was added osmium(VIII) oxide in H₂O (9.25 mL, 4 wt %, 3.63 mmol) dropwise at room temperature (15° C.) under an argon atmosphere. The reaction mixture was then stirred for 15 h at room temperature. The reaction mixture was quenched with saturated sodium thiosulfate (60 mL) under argon and stirred for 20 minutes at 0° C. The resulting mixture was then diluted with EtOAc/H₂O (600 mL/200 mL). The organic layer was separated and washed with H₂O (200 mL) and brine (2×200 mL), dried over anhydrous sodium sulfate, filtered, and the filtrate was concentrated under reduced pressure. The resulting crude material was purified by column chromatography on silica (0-60% EtOAc/PE) to afford (2S,3S,4R)-4-(4-chloro-7H-pyrrolo[2,3-d]pyrimidin-7-yl)-2,3-dihydroxy-2-methylcyclopentanone as a solid. MS: 282 (M+1).

Step 7: To a mixture of (2S,3S,4R)-4-(4-chloro-7H-pyrrolo[2,3-d]pyrimidin-7-yl)-2,3-dihydroxy-2-methylcyclopentanone (500 mg, 1.78 mmol) and 4-methylbenzenesulfonic acid (61.1 mg, 0.355 mmol) in anhydrous acetone (25 mL) was added 2,2-dimethoxypropane (2.77 g, 26.6 mmol) dropwise at room temperature under argon. The reaction mixture was stirred for 15 h at 30° C. The reaction was quenched with saturated sodium bicarbonate (20 mL) at 0° C. The resulting mixture was diluted with EtOAc/H₂O (200 mL/30 mL), and the organic layer was separated, washed with brine (80 mL), dried over anhydrous sodium sulfate, filtered, and the filtrate was concentrated under reduced pressure. The resulting residue was purified by column chromatography on silica (0-25% EtOAc/PE) to afford (3aS,6R,6aS)-6-(4-chloro-7H-pyrrolo[2,3-d]pyrimidin-7-yl)-2,2,3a-trimethyldihydro-3aH-cyclopenta[d][1,3]dioxol-4(5H)-one as a solid. MS: 322 (M+1).

Step 8: Cerium(III) chloride (2.45 g, 9.95 mmol) was suspended in THF (15 mL) and stirred for 0.5 h at room temperature under argon. To a second flame-dried round-bottom flask was added ethynyltrimethylsilane (977 mg, 9.95 mmol) in anhydrous THF (10 mL). The TMS-acetylene solution and the flask containing the CeCl₃ were both cooled to −78° C. To the TMS-acetylene solution, n-BuLi (3.98 mL, 2.5 M in hexane, 9.95 mmol) was added dropwise by syringe. Both mixtures were stirred for 20 minutes and then the lithium TMS-acetylide solution was transferred via canula into the rapidly-stirred CeCl₃ suspension. The mixture was stirred for 0.5 h at −78° C. (3aS,6R,6aS)-6-(4-chloro-7H-pyrrolo[2,3-d]pyrimidin-7-yl)-2,2,3a-trimethyldihydro-3aH-cyclopenta[d][1,3]dioxol-4(5H)-one (400 mg, 1.243 mmol) was dissolved in anhydrous THF (15 mL), cooled to −78° C., and transferred via canula into the flask containing the cerium acetylide salt. The resulting mixture was stirred for 2 h at −78° C. The reaction was quenched with saturated ammonium chloride (40 mL) at 0° C. and diluted with EtOAc/H₂O (200 mL/80 mL). The organic layer was separated and washed with saturated sodium bicarbonate (100 mL) and brine (100 mL). The organic layer was then dried over anhydrous sodium sulfate, filtered, and the filtrate was concentrated under reduced pressure. The residue was purified by column chromatography (0-18% EtOAc/PE) to afford (3aS,4S,6R,6aS)-6-(4-chloro-7H-pyrrolo[2,3-d]pyrimidin-7-yl)-2,2,3a-trimethyl-4-((trimethylsilyl)ethynyl)tetrahydro-3aH-cyclopenta[d][1,3]dioxol-4-ol as a solid. MS: 420 (M+1).

Step 9: To a solution of (3aS,4S,6R,6aS)-6-(4-chloro-7H-pyrrolo[2,3-d]pyrimidin-7-yl)-2,2,3a-trimethyl-4-((trimethylsilyl)ethynyl)tetrahydro-3aH-cyclopenta[d][1,3]dioxol-4-ol (220 mg, 0.524 mmol) in anhydrous THF (5 mL) cooled to 0° C. was added TBAF (1.05 mL, 1 M in THF, 1.048 mmol) dropwise under argon. The reaction mixture was stirred for 1 h at 0° C. Then the solvent was removed under reduced pressure. The resulting residue was purified by column chromatography on silica (0-20% EtOAc/PE) to afford (3aS,4S,6R,6aS)-6-(4-chloro-7H-pyrrolo[2,3-d]pyrimidin-7-yl)-4-ethynyl-2,2,3a-trimethyltetrahydro-3aH-cyclopenta[d][1,3]dioxol-4-ol as a solid. MS: 348 (M+1).

Step 10: (3aS,4S,6R,6aS)-6-(4-chloro-7H-pyrrolo[2,3-d]pyrimidin-7-yl)-4-ethynyl-2,2,3a-trimethyltetrahydro-3aH-cyclopenta[d][1,3]dioxol-4-ol (170 mg, 0.489 mmol) was dissolved in NH₃ (15 mL, 20% in iPrOH) at −70° C. The reaction was stirred for 15 h at 90° C. in a sealed tube. Then the solvent was removed under reduced pressure. The resulting residue was purified by column chromatography on silica (0-8% MeOH/DCM) to afford (3aS,4S,6R,6aS)-6-(4-amino-7H-pyrrolo[2,3-d]pyrimidin-7-yl)-4-ethynyl-2,2,3a-trimethyltetrahydro-3aH-cyclopenta[d][1,3]dioxol-4-ol as a solid. MS: 329 (M+1).

Step 11: A solution of (3aS,4S,6R,6aS)-6-(4-amino-7H-pyrrolo[2,3-d]pyrimidin-7-yl)-4-ethynyl-2,2,3a-trimethyltetrahydro-3aH-cyclopenta[d][1,3]dioxol-4-ol (150 mg, 0.457 mmol) in anhydrous MeOH (4 mL) was reduced under a hydrogen atmosphere using Lindlar Catalyst (22.5 mg, 10.57 μmol). The reaction mixture was stirred for 2.5 h at 30° C. The reaction mixture was then filtered through Celite, and the filtrate was concentrated under reduced pressure. The resulting residue was purified by chiral HPLC (EtOH/hexanes with 8 mM NH₃-MeOH modifier) to afford (3aS,4S,6R,6aS)-6-(4-amino-7H-pyrrolo[2,3-d]pyrimidin-7-yl)-2,2,3a-trimethyl-4-vinyltetrahydro-3aH-cyclopenta[d][1,3]dioxol-4-ol as an oil. MS: 331 (M+1).

Step 12: Under an argon atmosphere, (3aS,4S,6R,6aS)-6-(4-amino-7H-pyrrolo[2,3-d]pyrimidin-7-yl)-2,2,3a-trimethyl-4-vinyltetrahydro-3aH-cyclopenta[d][1,3]dioxol-4-ol (70 mg, 0.212 mmol) was dissolved in 9-BBN solution (2.12 mL, 0.5 M in THF, 1.06 mmol) at room temperature, and the mixture was stirred for 1 h at 60° C. The reaction was cooled to 0° C., and a solution of K₃PO₄ (183 mg, 1.059 mmol) in H₂O (2 mL) was added. The mixture was stirred for 0.5 h at room temperature. Then a solution of 3-bromo-7-iodo-N-(4-methoxybenzyl)quinolin-2-amine (99 mg, 0.212 mmol) and Pd(dppf)Cl₂ (26.0 mg, 0.032 mmol) in anhydrous THF (2.5 mL) were added to the mixture. The resulting mixture was irradiated with microwave radiation at 70° C. for 2 h. The mixture was concentrated under reduced pressure, and the resulting residue was dissolved in EtOAc (100 mL) and then washed with H₂O (30 mL) and brine (2×30 mL). The organic layer was dried over anhydrous sodium sulfate, filtered, and the filtrate was concentrated under reduced pressure. The resulting crude mixture was purified by column chromatography on silica (0-3% MeOH/DCM) to afford (3aS,4S,6R,6aS)-6-(4-amino-7H-pyrrolo[2,3-d]pyrimidin-7-yl)-4-(2-(3-bromo-2-((4-methoxybenzyl)amino)quinolin-7-yl)ethyl)-2,2,3a-trimethyltetrahydro-3aH-cyclopenta[d][1,3]dioxol-4-ol as a solid. MS: 673/675 (M+1/M+3).

Step 13: Under an argon atmosphere, (3aS,4S,6R,6aS)-6-(4-amino-7H-pyrrolo[2,3-d]pyrimidin-7-yl)-4-(2-(3-bromo-2-((4-methoxybenzyl)amino)quinolin-7-yl)ethyl)-2,2,3a-trimethyltetrahydro-3aH-cyclopenta[d][1,3]dioxol-4-ol (100 mg, 0.148 mmol) was dissolved in TFA (3.0 mL, 38.9 mmol) at room temperature. The reaction mixture was stirred for 4 h at 60° C. The mixture was then evaporated under reduced pressure. The resulting residue was co-evaporated with toluene (3×90 mL). This residue was then purified by reverse phase column chromatography (ACN/water). The product was further purified by reverse phase HPLC (ACN/water with 10 mM NH₄HCO₃ modifier) to afford (1R,2S,3S,4R)-1-(2-(2-amino-3-bromoquinolin-7-yl)ethyl)-4-(4-amino-7H-pyrrolo[2,3-d]pyrimidin-7-yl)-2-methylcyclopentane-1,2,3-triol as a solid. MS: 513/515 (M+1/M+3). ¹H-NMR (400 MHz, DMSO-d₆+10% D₂O) δ 8.66 (s, 1H), 8.36 (s, 1H), 7.75 (d, J=8.4 Hz, 1H), 7.62 (d, J=3.6 Hz, 1H), 7.50 (s, 1H), 7.33 (d, J=8.0 Hz, 1H), 7.00-6.97 (m, 1H), 5.06 (dd, J=9.6, 18.4 Hz, 1H), 4.06 (d, J=8.8 Hz, 1H), 2.96-2.77 (m, 2H), 2.23-2.12 (m, 2H), 1.98-1.81 (m, 2H), 1.38 (s, 3H).

### Example 119

### (1S,2R,3aR,4S,6aR)-4-((2-amino-3-bromoquinolin-7-yl)oxy)-2-(4-amino-7H-pyrrolo[2,3-d]pyrimidin-7-yl)hexahydropentalene-1,6a(1H)-diol 2,2,2-trifluoroacetate

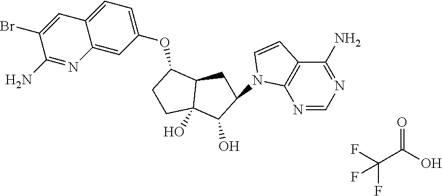 [SMILES: Nc1nc2cc(O[C@H]3CC[C@@]4(O)[C@@H]3C[C@@H](n3ccc5c(N)ncnc53)[C@@H]4O)ccc2cc1Br ; O=C(O)C(F)(F)F]

Step 1: To an oven-dried, argon-purged vial with (3aS,4R,5aR,6S,8aR)-4-(4-chloro-7H-pyrrolo[2,3-d]pyrimidin-7-yl)-2,2-dimethylhexahydro-5H-pentaleno[1,6a-d][1,3]dioxol-6-ol (141.4 mg, 0.404 mmol) was added 3-bromo-7-iodo-N-(4-methoxybenzyl)quinolin-2-amine (379 mg, 0.808 mmol), 1,10-phenanthroline (29.1 mg, 0.162 mmol), cuprous iodide (15.40 mg, 0.081 mmol), and cesium carbonate (395 mg, 1.213 mmol). The solids were dissolved in xylene (4 mL), and the reaction was heated to 140° C. for 18 h under argon. The reaction was slowly cooled to room temperature. The reaction was filtered through Celite, and then concentrated under reduced pressure. The residue was purified by column chromatography on silica (20-35-80% EtOAc/hexanes) to afford 3-bromo-7-(((3aS,4R,5aR,6S,8aR)-4-(4-chloro-7H-pyrrolo[2,3-d]pyrimidin-7-yl)-2,2-dimethylhexahydro-3aH-pentaleno[1,6a-d][1,3]dioxol-6-yl)oxy)-N-(4-methoxybenzyl)quinolin-2-amine as a solid. MS: 690/692 (M+1/M+3).

Step 2: To a vial with 3-bromo-7-(((3aS,4R,5aR,6S,8aR)-4-(4-chloro-7H-pyrrolo[2,3-d]pyrimidin-7-yl)-2,2-dimethylhexahydro-3aH-pentaleno[1,6a-d][1,3]dioxol-6-yl)oxy)-N-(4-methoxybenzyl)quinolin-2-amine (37.4 mg, 0.054 mmol) was added ammonia (3.5 mL, 24.50 mmol, 7N in MeOH). The reaction was heated in the microwave at 140° C. for 5 h. The reaction was concentrated under reduced pressure, and the resulting crude 7-(((3aS,4R,5aR,6S,8aR)-4-(4-amino-7H-pyrrolo[2,3-d]pyrimidin-7-yl)-2,2-dimethylhexahydro-3aH-pentaleno[1,6a-d][1,3]dioxol-6-yl)oxy)-3-bromo-N-(4-methoxybenzyl)quinolin-2-amine was carried directly to the next step.

Step 3: To a vial containing crude 7-(((3aS,4R,5aR,6S,8aR)-4-(4-amino-7H-pyrrolo[2,3-d]pyrimidin-7-yl)-2,2-dimethylhexahydro-3aH-pentaleno[1,6a-d][1,3]dioxol-6-yl)oxy)-3-bromo-N-(4-methoxybenzyl)quinolin-2-amine (25 mg, 0.037 mmol) was added DCM (0.5 mL), followed by TFA (0.4 mL, 5.19 mmol). The reaction was stirred at 40° C. for 75 minutes. Then water (0.1 mL) was added, along with more DCM (0.1 mL), and the reaction was stirred at 40° C. for 2.5 h in total. Then more DCM (0.2 mL), more TFA (0.3 mL), and more water (0.1 mL) were added, and the reaction was heated to 40° C. for another 2 h. Finally, anisole (100 μl, 0.915 mmol) was added, and the reaction was stirred at 40° C. for 1 h. The reaction was concentrated under reduced pressure, dissolved in DMSO, filtered, and submitted for mass-triggered reverse phase HPLC (MeCN/water with 0.1% TFA modifier) to afford (1S,2R,3aR,4S,6aR)-4-((2-amino-3-bromoquinolin-7-yl)oxy)-2-(4-amino-7H-pyrrolo[2,3-d]pyrimidin-7-yl)octahydropentalene-1,6a-diol as the TFA salt as a solid. MS: 511/513 (M+1/M+3). ¹H NMR (600 MHz, DMSO-d₆) δ 8.72 (s, 1H), 8.37 (s, 1H), 7.82 (d, J=3.6 Hz, 1H), 7.78 (d, J=8.9 Hz, 1H), 7.07 (dd, J=8.9, 2.0 Hz, 1H), 6.98-6.94 (m, 2H), 4.96-4.89 (m, 1H), 4.56 (d, J=4.2 Hz, 1H), 4.05 (d, J=10.5 Hz, 1H), 2.58-2.52 (m, 1H), 2.36-2.27 (m, 2H), 2.09-2.03 (m, 1H), 1.98-1.87 (m, 2H), 1.75-1.67 (m, 1H).

### Example 120

### (2R,3R,3aS,6S,6aR)-6-((2-amino-3-fluoroquinolin-7-yl)amino)-2-(4-methyl-7H-pyrrolo[2,3-d]pyrimidin-7-yl)hexahydro-3aH-cyclopenta[b]furan-3,3a-diol

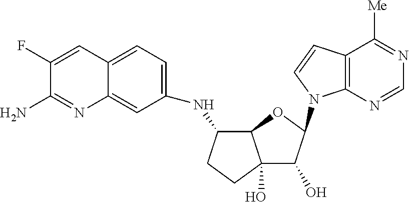 [SMILES: Cc1ncnc2c1ccn2[C@@H]1O[C@@H]2[C@@H](Nc3ccc4cc(F)c(N)nc4c3)CC[C@]2(O)[C@H]1O]

Step 1: To a mixture of (3aR,4R,5aR,6R,8aR)-2,2-dimethyl-4-(4-methyl-7H-pyrrolo[2,3-d]pyrimidin-7-yl)hexahydrocyclopenta[2,3]furo[3,4-d][1,3]dioxol-6-ol (200 mg, 0.604 mmol) and triphenylphosphine (317 mg, 1.21 mmol) in toluene (2.5 mL) was added isoindoline-1,3-dione (178 mg, 1.21 mmol) at room temperature. Then the mixture was cooled to 0° C. and DIAD (0.235 mL, 1.21 mmol) was added dropwise. The resulting mixture was stirred for 1.5 h at 80° C. The reaction mixture was then concentrated under reduced pressure, and the resulting residue was purified by Prep-TLC (1:1 EtOAc: PE) to afford 2-((3aR,4R,5aR,6S,8aR)-2,2-dimethyl-4-(4-methyl-7H-pyrrolo[2,3-d]pyrimidin-7-yl)hexahydrocyclopenta[2,3]furo[3,4-d][1,3]dioxol-6-yl)isoindoline-1,3-dione as a solid. MS: 461 (M+1).

Step 2: To a stirred solution of 2-((3aR,4R,5aR,6S,8aR)-2,2-dimethyl-4-(4-methyl-7H-pyrrolo[2,3-d]pyrimidin-7-yl)hexahydrocyclopenta[2,3]furo[3,4-d][1,3]dioxol-6-yl)isoindoline-1,3-dione (220 mg, 0.478 mmol) in MeOH (1.5 mL) was added hydrazine hydrate (598 mg, 9.56 mmol). The resulting solution was stirred at room temperature for overnight. The reaction mixture was concentrated under reduced pressure, and the resulting solid was suspended in DCM (30 mL). The mixture was filtered, and the filtrate was concentrated under reduced pressure to afford (3aR,4R,5aR,6S,8aR)-2,2-dimethyl-4-(4-methyl-7H-pyrrolo[2,3-d]pyrimidin-7-yl)hexahydrocyclopenta[2,3]furo[3,4-d][1,3]dioxol-6-amine as an oil. MS: 331 (M+1).

Step 3: To a stirred solution of (3aR,4R,5aR,6S,8aR)-2,2-dimethyl-4-(4-methyl-7H-pyrrolo[2,3-d]pyrimidin-7-yl)hexahydrocyclopenta[2,3]furo[3,4-d][1,3]dioxol-6-amine (40 mg, 0.097 mmol) and 7-bromo-N-(2,4-dimethoxybenzyl)-3-fluoroquinolin-2-amine (41.7 mg, 0.107 mmol) in THF (0.5 mL) were added Xantphos Pd G3 (4.59 mg, 4.84 μmol) and sodium 2-methylpropan-2-olate (27.9 mg, 0.291 mmol). The resulting mixture was stirred at 50° C. for 16 h. The reaction was quenched with saturated aqueous ammonium chloride (25 mL) and extracted with EtOAc (3×40 mL). The combined organic layers were washed with brine (30 mL), dried over anhydrous sodium sulfate, and filtered. The filtrate was concentrated under reduced pressure, and the residue was purified by Prep-TLC (2:1 EtOAc: PE) to afford N2-(2,4-dimethoxybenzyl)-N7-((3aR,4R,5aR,6S,8aR)-2,2-dimethyl-4-(4-methyl-7H-pyrrolo[2,3-d]pyrimidin-7-yl)hexahydrocyclopenta[2,3]furo[3,4-d][1,3]dioxol-6-yl)-3-fluoroquinoline-2,7-diamine as a solid. MS: 641 (M+1).

Step 4: N2-(2,4-dimethoxybenzyl)-N7-((3aR,4R,5aR,6S,8aR)-2,2-dimethyl-4-(4-methyl-7H-pyrrolo[2,3-d]pyrimidin-7-yl)hexahydrocyclopenta[2,3]furo[3,4-d][1,3]dioxol-6-yl)-3-fluoroquinoline-2,7-diamine (58 mg, 0.091 mmol) was dissolved in TFA (2 mL, 26.0 mmol). The resulting solution was stirred at 50° C. for overnight. The reaction mixture was then co-evaporated with toluene (2×5 mL) under reduced pressure. The resulting residue was purified by reverse phase column chromatography (ACN/water with 5 mM NH₄HCO₃ modifier). The product was further purified by prep-TLC (10:1 DCM/MeOH) followed by reverse phase column chromatography (ACN/water with 5 mM NH₄HCO₃ modifier) to afford (2R,3R,3aS,6S,6aR)-6-((2-amino-3-fluoroquinolin-7-yl)amino)-2-(4-methyl-7H-pyrrolo[2,3-d]pyrimidin-7-yl)hexahydro-2H-cyclopenta[b]furan-3,3a-diol as a solid. MS: 451 (M+1). ¹H-NMR (300 MHz, DMSO-d₆) δ 8.69 (s, 1H), 7.92 (d, J=3.6 Hz, 1H), 7.53 (d, J=12.0 Hz, 1H), 7.32 (d, J=8.7 Hz, 1H), 6.83 (d, J=3.6 Hz, 1H), 6.69 (d, J=7.8 Hz, 1H), 6.42-6.36 (m, 3H), 6.8 (d, J=4.5 Hz, 1H), 6.11 (d, J=8.1 Hz, 1H), 5.38 (d, J=7.2 Hz, 1H), 5.23 (s, 1H), 4.42 (d, J=7.8 Hz, 1H), 3.96 (s, 1H), 3.55-3.53 (m, 1H), 2.69 (s, 3H), 2.50-2.49 (m, 1H), 2.07-2.02 (m, 2H), 1.94-1.90 (m, 1H).

### Example 121

### (2R,3R,3aS,6S,6aR)-6-((2-amino-3-fluoroquinolin-7-yl)methyl)-2-(4-amino-7H-pyrrolo[2,3-d]pyrimidin-7-yl)-6a-methylhexahydro-3aH-cyclopenta[b]furan-3,3a-diol

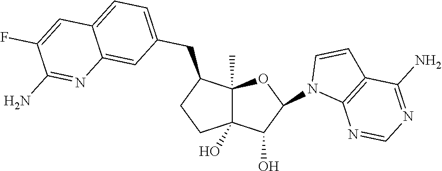 [SMILES: C[C@]12O[C@@H](n3ccc4c(N)ncnc43)[C@H](O)[C@@]1(O)CC[C@H]2Cc1ccc2cc(F)c(N)nc2c1]

Step 1: To a solution of (S)-2-methyl-CBS-oxazaborolidine (1.788 g, 6.45 mmol) in THF (31 mL) was added borane-THF complex (6.45 mL, 1M in THF, 6.45 mmol) dropwise at 0° C. The reaction was stirred for 30 minutes at 0° C. A solution of 2-methylcyclopent-2-enone (3.1 g, 32.2 mmol) in THF (25 mL) and borane-THF complex (22.57 mL, 1M in THF, 22.57 mmol) were added simultaneously dropwise at 0° C. The reaction mixture was warmed slowly to room temperature and stirred for 1.5 h. The reaction mixture was carefully quenched by addition of 180 mL of water at 0° C. The mixture was stirred for 0.5 h at room temperature, and then extracted with DCM (200 mL). The organic extract was washed with saturated aqueous ammonium chloride (100 mL) and brine (100 mL), dried with anhydrous magnesium sulfate, filtered, and the filtrate was concentrated under reduced pressure to afford (R)-2-methylcyclopent-2-enol as an oil, which was used in the next step without further purification.

Step 2: To a stirred solution of (R)-2-methylcyclopent-2-enol (1 g, 10.19 mmol) in DCM (10 mL) were added DMAP (1.867 g, 15.28 mmol) and triethylamine (1.562 mL, 11.21 mmol) at 0° C. under an argon atmosphere. Then acetic anhydride (2.08 g, 20.4 mmol) was added slowly. The mixture was stirred at 0° C. for 1 h. The mixture was quenched with H₂O (50 mL) and extracted with DCM (60 mL×2). The combined organic layers were washed with brine (40 mL), dried over anhydrous sodium sulfate, and filtered. The filtrate was concentrated under reduced pressure. The resulting residue was purified by column chromatography on silica (0-15% EtOAc/PE) to afford (R)-2-methylcyclopent-2-en-1-yl acetate as an oil. Then a solution of (R)-2-methylcyclopent-2-en-1-yl acetate (700 mg, 4.99 mmol) in THF (8 mL) was added to lithium diisopropylamide (3.99 mL, 2M in THF/heptane, 7.99 mmol) at −78° C. over 3 minutes. Then a solution of tert-butylchlorodimethylsilane (1.43 g, 9.49 mmol) in THF (2 mL) was added and the mixture was stirred at −78° C. for 20 minutes. The reaction mixture was warmed to room temperature and stirred for an additional 2 h. The solution was then heated at reflux overnight, cooled to 0° C. and treated with concentrated HCl (2 mL). The mixture was stirred at 0° C. for 1 h. The reaction mixture was then partitioned between diethyl ether (40 mL) and water (20 mL). The aqueous layer was extracted with diethyl ether (30 mL). The combined organic layers were dried with anhydrous sodium sulfate and filtered. The filtrate was then concentrated under reduced pressure, and the residue was purified by column chromatography on silica (0-30% EtOAc/PE) to afford (R)-2-(2-methylcyclopent-2-en-1-yl)acetic acid as an oil. ¹H NMR (400 MHz, DMSO-d₆) δ 12.04 (s, 1H), 5.36-5.34 (m, 1H), 5.26 (s, 1H), 2.77-2.75 (m, 1H), 2.46-2.41 (m, 1H), 2.30-1.91 (m, 3H), 1.64 (s, 3H), 1.51-1.46 (m, 1H).

Step 3: To a solution of (R)-2-(2-methylcyclopent-2-en-1-yl)acetic acid (10 g, 71.3 mmol) in tert-butyl alcohol (100 mL) were added tetraoxotungsten(X)hydride (1.78 g, 7.13 mmol) and hydrogen peroxide (18.2 mL, 30% in water, 178 mmol). The resulting suspension was stirred at 80° C. for 30 minutes, and then the reaction mixture was cooled to 0° C. before it was quenched with saturated aqueous sodium thiosulfate (100 mL). The mixture was stirred for 1 h at room temperature and diluted with EtOAc (200 mL). The organic phases were separated, and the aqueous layer was extracted with EtOAc (4×100 mL). The combined organic phases were washed with brine (100 mL), dried over anhydrous sodium sulfate, filtered, and concentrated under reduced pressure. The residue was purified by column chromatography on silica (0-60% EtOAc/PE) to afford (3aR,6S,6aS)-6-hydroxy-6a-methylhexahydro-2H-cyclopenta[b]furan-2-one as an oil. ¹H NMR (400 MHz, DMSO-d₆) δ 5.09 (d, J=4.4 Hz, 1H), 3.89-3.86 (m, 1H), 3.03-2.87 (m, 1H), 2.46-2.41 (m, 1H), 2.29-2.29 (m, 1H), 2.11-1.99 (m, 1H), 1.83-1.72 (m, 1H), 1.59-1.48 (m, 1H), 1.40-1.23 (m, 4H).

Step 4: To a stirred solution of (3aR,6S,6aS)-6-hydroxy-6a-methylhexahydro-2H-cyclopenta[b]furan-2-one (6 g, 38.4 mmol) in DMF (40 mL) were added 1H-imidazole (7.85 g, 115 mmol) and tert-butylchlorodiphenylsilane (12.7 g, 46.1 mmol) at 0° C. under an argon atmosphere. The resulting mixture was stirred at 25° C. for 16 h. The reaction mixture was then quenched with saturated aqueous Na₂CO₃ (100 mL) and extracted with EtOAc (200 mL×2). The combined organic layers were washed with water (2×100 mL), followed by brine (100 mL), dried over anhydrous sodium sulfate, and filtered. The filtrate was concentrated under reduced pressure. The resulting residue was purified by column chromatography on silica (0-15% EtOAc/PE) to afford (3aR,6S,6aS)-6-((tert-butyldiphenylsilyl)oxy)-6a-methylhexahydro-2H-cyclopenta[b]furan-2-one as an oil. MS: 395 (M+1). ¹H-NMR (400 MHz, DMSO-d₆) δ 7.65-7.60 (m, 4H), 7.56-7.37 (m, 6H), 4.11 (t, J=5.6 Hz, 1H), 2.97-2.88 (m, 1H), 2.57-2.51 (m, 1H), 2.28-2.23 (m, 1H), 2.05-1.96 (m, 1H), 1.61-1.48 (m, 1H), 1.46-1.39 (m, 4H), 1.32-1.19 (m, 1H), 1.04 (s, 9H).

Step 5: To a solution of (3aR,6S,6aS)-6-((tert-butyldiphenylsilyl)oxy)-6a-methylhexahydro-2H-cyclopenta[b]furan-2-one (13 g, 32.9 mmol) in THF (40 mL) was added chlorotrimethylsilane (17.9 g, 165 mmol) at −78° C. Lithium bis(trimethylsilyl)amide (49.4 mL, 1M in THF, 49.4 mmol) was added dropwise over 5 minutes. The resulting mixture was stirred at −78° C. for 30 minutes. A solution of phenyl hypochloroselenoite (7.57 g, 39.5 mmol) in THF (15 mL) was added to the reaction mixture, and the mixture was stirred at −78° C. for 2 h. The reaction mixture was then quenched with saturated aqueous ammonium chloride (100 mL) and extracted with EtOAc (150 mL×3). The combined organic layers were washed with brine (100 mL), dried over anhydrous sodium sulfate, and filtered. The filtrate was concentrated under reduced pressure to afford crude (3R,3aS,6S,6aS)-6-((tert-butyldiphenylsilyl)oxy)-6a-methyl-3-(phenylselanyl)hexahydro-2H-cyclopenta[b]furan-2-one as an oil which was used directly in the next step.

Step 6: To a solution of crude (3R,3aS,6S,6aS)-6-((tert-butyldiphenylsilyl)oxy)-6a-methyl-3-(phenylselanyl)hexahydro-2H-cyclopenta[b]furan-2-one (18.08 g, 32.9 mmol) in DCM (250 mL) was added hydrogen peroxide (18.7 g, 30% in water, 165 mmol) at room temperature. The reaction mixture was stirred at room temperature for 1 h. The mixture was then concentrated under reduced pressure, and the resulting residue was purified by column chromatography on silica (1-20% EtOAc/PE) to afford (6S,6aS)-6-((tert-butyldiphenylsilyl)oxy)-6a-methyl-4,5,6,6a-tetrahydro-2H-cyclopenta[b]furan-2-one as an oil. MS: 410 (M+NH₄). ¹H NMR (400 MHz, DMSO-d₆) δ 7.64-7.59 (m, 4H), 7.52-7.39 (m, 6H), 5.82-5.81 (m, 1H), 3.85 (t, J=8.8 Hz, 1H), 2.86-2.67 (m, 1H), 2.44-2.30 (m, 1H), 2.11-2.01 (m, 1H), 1.97-1.84 (m, 1H), 1.52 (s, 3H), 1.05 (s, 9H).

Step 7: A solution of (6S,6aS)-6-((tert-butyldiphenylsilyl)oxy)-6a-methyl-4,5,6,6a-tetrahydro-2H-cyclopenta[b]furan-2-one (8 g, 20.4 mmol) in MeCN (20 mL) was suspended in a solution of 2-hydroxypropane-1,2,3-tricarboxylic acid (7.83 g, 40.8 mmol) in water (15 mL). Then potassium osmate(VI) dihydrate (0.375 g, 1.02 mmol) was added, followed by 4-methylmorpholine N-oxide (4.77 mL, 50 wt % in water, 22.4 mmol). The reaction mixture was stirred at room temperature for 16 h. To the reaction mixture was then added water (100 mL), and it was extracted with EtOAc (150 mL×3). The combined organic layers were washed with brine (100 mL), dried over anhydrous sodium sulfate, and filtered. The filtrate was concentrated under reduced pressure, and the resulting residue was purified by column chromatography on silica (1-50% EtOAc/PE) to afford (3R,3aS,6S,6aR)-6-((tert-butyldiphenylsilyl)oxy)-3,3a-dihydroxy-6a-methylhexahydro-2H-cyclopenta[b]furan-2-one as an oil. MS: 444 (M+NH₄). ¹H-NMR (300 MHz, DMSO-d₆) δ 7.66-7.61 (m, 4H), 7.52-7.41 (m, 6H), 5.99 (d, J=7.2 Hz, 1H), 5.22 (s, 1H), 4.23 (t, J=7.5 Hz, 1H), 4.07 (d, J=7.2 Hz, 1H), 1.87-1.68 (m, 2H), 1.65-1.51 (m, 2H), 1.35 (s, 3H), 1.05 (s, 9H).

Step 8: To a stirred solution of (3R,3aS,6S,6aR)-6-((tert-butyldiphenylsilyl)oxy)-3,3a-dihydroxy-6a-methylhexahydro-2H-cyclopenta[b]furan-2-one (7 g, 16.4 mmol) and chlorobis(cyclooctene)iridium(I) dimer (0.147 g, 0.164 mmol) in DCM (13 mL) was added diethylsilane (2.17 g, 24.6 mmol) dropwise under an argon atmosphere at room temperature. The resulting solution was stirred at room temperature for 2 h. Solid tetrabutylammonium fluoride trihydrate (5.18 g, 16.4 mmol) was then added to the reaction mixture. The mixture was stirred at room temperature for 16 h. The reaction was quenched with saturated aqueous sodium bicarbonate (100 mL) and extracted with DCM (3×100 mL). The combined organic layers were washed with brine (100 mL), dried over anhydrous sodium sulfate, and filtered. The filtrate was concentrated under reduced pressure. The resulting residue was purified by column chromatography on silica (1-50% EtOAc/PE) to afford (3R,3aS,6S,6aR)-6-((tert-butyldiphenylsilyl)oxy)-6a-methylhexahydro-2H-cyclopenta[b]furan-2,3,3a-triol as an oil. MS: 446 (M+NH₄).

Step 9: To a stirred solution of (3R,3aS,6S,6aR)-6-((tert-butyldiphenylsilyl)oxy)-6a-methylhexahydro-2H-cyclopenta[b]furan-2,3,3a-triol (5.5 g, 12.8 mmol) in dry MeCN (260 mL) under the an argon atmosphere was added (E)-diazene-1,2-diylbis(piperidin-1-ylmethanone) (4.86 g, 19.3 mmol) at 0° C., followed by tributylphosphine (5.13 mL, 20.5 mmol) at 0° C. The resulting mixture was stirred at 35° C. for about 1 h. Separately, to a stirred solution of 4-chloro-7H-pyrrolo[2,3-d]pyrimidine (3.74 g, 24.4 mmol) in dry MeCN (25 mL) under an argon atmosphere was added DBU (3.48 mL, 23.1 mmol) at room temperature. The solution was stirred at room temperature for 30 minutes, and then this solution was transferred to the reaction mixture originally containing the triol by means of a syringe, and the resulting reaction was stirred at 35° C. for 1 h. The reaction mixture was quenched with saturated aqueous ammonium chloride (100 mL) and extracted with EtOAc (3×200 mL). The combined organic layers were washed with brine (200 mL), dried over anhydrous sodium sulfate, and filtered. The filtrate was concentrated under reduced pressure, and the residue was purified by reverse phase column chromatography (ACN/water with 5 mM NH₄HCO₃ modifier) to afford (2R,3R,3aS,6S,6aR)-6-((tert-butyldiphenylsilyl)oxy)-2-(4-chloro-7H-pyrrolo[2,3-d]pyrimidin-7-yl)-6a-methylhexahydro-2H-cyclopenta[b]furan-3,3a-diol as a solid. MS: 564 (M+1).

Step 10: To a solution of (2R,3R,3aS,6S,6aR)-6-((tert-butyldiphenylsilyl)oxy)-2-(4-chloro-7H-pyrrolo[2,3-d]pyrimidin-7-yl)-6a-methylhexahydro-2H-cyclopenta[b]furan-3,3a-diol (4 g, 7.09 mmol) in acetone (40 mL) were added 4-methylbenzenesulfonic acid (0.122 g, 0.709 mmol) and 2,2-dimethoxypropane (7.38 g, 70.9 mmol) at ambient temperature under argon. The mixture was stirred at room temperature for 16 h. The mixture was concentrated under reduced pressure, and the resulting residue was purified by column chromatography on silica (0-20% EtOAc/PE) to afford 7-((3aR,4R,5aR,6S,8aS)-6-((tert-butyldiphenylsilyl)oxy)-2,2,5a-trimethylhexahydrocyclopenta[2,3]furo[3,4-d][1,3]dioxol-4-yl)-4-chloro-7H-pyrrolo[2,3-d]pyrimidine as a solid. MS: 604 (M+1).

Step 11: To a solution of 7-((3aR,4R,5aR,6S,8aS)-6-((tert-butyldiphenylsilyl)oxy)-2,2,5a-trimethylhexahydrocyclopenta[2,3]furo[3,4-d][1,3]dioxol-4-yl)-4-chloro-7H-pyrrolo[2,3-d]pyrimidine (3.8 g, 6.29 mmol) in THF (20 mL) under an argon atmosphere was added tetrabutylammonium fluoride (12.6 mL, 1M in THF, 12.6 mmol) at 0° C. The reaction mixture was then stirred at room temperature for 16 h and concentrated under reduced pressure. The resulting residue was purified by column chromatography on silica (0-50% EtOAc/PE). The product was further purified by PrepSFC (CHIRALPAK IF, 40% 8 mM NH₃ in MeOH in CO₂) to afford (3aR,4R,5aR,6S,8aS)-4-(4-chloro-7H-pyrrolo[2,3-d]pyrimidin-7-yl)-2,2,5a-trimethylhexahydrocyclopenta[2,3]furo[3,4-d][1,3]dioxol-6-ol as a solid. MS: 366 (M+1).

Step 12: Dess-Martin periodinane (696 mg, 1.640 mmol) was added portion wise to a stirred solution of (3aR,4R,5aR,6S,8aS)-4-(4-chloro-7H-pyrrolo[2,3-d]pyrimidin-7-yl)-2,2,5a-trimethylhexahydrocyclopenta[2,3]furo[3,4-d][1,3]dioxol-6-ol (400 mg, 1.093 mmol) in DCM (15 mL) at 0° C. The reaction mixture was then stirred at room temperature for 2 h. The reaction mixture was quenched with saturated aqueous sodium bicarbonate (100 mL) and extracted with EtOAc (100 mL×3). The combined organic layers were washed with brine (100 mL), dried over anhydrous sodium sulfate, and filtered. The filtrate was concentrated under reduced pressure, and the resulting residue was purified by column chromatography on silica (1-30% EtOAc/PE) to afford (3aR,4R,5aS,8aS)-4-(4-chloro-7H-pyrrolo[2,3-d]pyrimidin-7-yl)-2,2,5a-trimethyltetrahydrocyclopenta[2,3]furo[3,4-d][1,3]dioxol-6(5aH)-one as a solid. MS: 364 (M+1).

Step 13: To a mixture of Nysted Reagent (4.51 g, 20% in THF, 1.98 mmol) in anhydrous THF (20 mL) was added TiCl₄ (1.98 mL, 1M in DCM, 1.98 mmol) dropwise at 0° C. under argon. The mixture was stirred at 0° C. for 5 minutes. Then a solution of (3aR,4R,5aS,8aS)-4-(4-chloro-7H-pyrrolo[2,3-d]pyrimidin-7-yl)-2,2,5a-trimethyltetrahydrocyclopenta[2,3]furo[3,4-d][1,3]dioxol-6(5aH)-one (360 mg, 0.990 mmol) in anhydrous THF (10 mL) was added at 0° C. The resulting mixture was stirred at room temperature for 3 h. The reaction was quenched with saturated sodium bicarbonate (100 mL) at 0° C. The resulting mixture was diluted with EtOAc (100 mL) at room temperature and extracted, and the aqueous layer was then re-extracted with EtOAc (100 mL×2). The combined organic layers were washed with H₂O (80 mL) and brine (80 mL), dried over anhydrous sodium sulfate, filtered, and the filtrate was concentrated under reduced pressure. The resulting residue was purified by column chromatography on silica (1-20% EtOAc/PE) to afford 4-chloro-7-((3aR,4R,5aR,8aS)-2,2,5a-trimethyl-6-methylenehexahydrocyclopenta[2,3]furo[3,4-d][1,3]dioxol-4-yl)-7H-pyrrolo[2,3-d]pyrimidine as a solid. MS: 362 (M+1).

Step 14: To a mixture of 4-chloro-7-((3aR,4R,5aR,8aS)-2,2,5a-trimethyl-6-methylenehexahydrocyclopenta[2,3]furo[3,4-d][1,3]dioxol-4-yl)-7H-pyrrolo[2,3-d]pyrimidine (240 mg, 0.663 mmol) in 1,4-dioxane (8 mL) was added ammonia hydrate (8 mL, 28 wt %, 0.663 mmol) at room temperature. The reaction mixture was then stirred at 90° C. for 15 h in a sealed tube. The solvent was removed under reduced pressure, and the resulting residue was purified by column chromatography on silica (1-10% MeOH/DCM) to afford 7-((3aR,4R,5aR,8aS)-2,2,5a-trimethyl-6-methylenehexahydrocyclopenta[2,3]furo[3,4-d][1,3]dioxol-4-yl)-7H-pyrrolo[2,3-d]pyrimidin-4-amine as a solid. MS: 343 (M+1).

Step 15: 7-((3aR,4R,5aR,8aS)-2,2,5a-trimethyl-6-methylenehexahydrocyclopenta[2,3]furo[3,4-d][1,3]dioxol-4-yl)-7H-pyrrolo[2,3-d]pyrimidin-4-amine (200 mg, 0.584 mmol) was co-evaporated with THF (3 mL) three times. Then 9-BBN solution (5.84 mL, 0.5M in THF, 2.92 mmol) was added at room temperature under argon. The reaction solution was stirred at 60° C. for 1 h. A solution of potassium phosphate (620 mg, 2.92 mmol) in Water (2.9 mL) was added dropwise at 0° C. under argon. The reaction solution was stirred at room temperature for 0.5 h. Then 7-bromo-3-fluoroquinolin-2-amine (148 mg, 0.613 mmol) in THF (2.9 mL) and [1,1′-bis(diphenylphosphino)ferrocene]dichloropalladium(II) (42.7 mg, 0.058 mmol) were added at room temperature. The reaction was irradiated with microwave radiation at 80° C. for 2.5 h. The reaction mixture was then cooled to room temperature, diluted with water (20 mL), and extracted with EtOAc (3×50 mL). The combined organic layers were concentrated under reduced pressure, and the resulting residue was purified by reverse phase HPLC (ACN/water with 5 mM NH₄HCO₃ modifier) to afford 7-(((3aR,4R,5aR,6S,8aS)-4-(4-amino-7H-pyrrolo[2,3-d]pyrimidin-7-yl)-2,2,5a-trimethylhexahydrocyclopenta[2,3]furo[3,4-d][1,3]dioxol-6-yl)methyl)-3-fluoroquinolin-2-amine as a solid. MS: 505 (M+1).

Step 16: To a 40 mL vial containing a solution of 7-(((3aR,4R,5aR,6S,8aS)-4-(4-amino-7H-pyrrolo[2,3-d]pyrimidin-7-yl)-2,2,5a-trimethylhexahydrocyclopenta[2,3]furo[3,4-d][1,3]dioxol-6-yl)methyl)-3-fluoroquinolin-2-amine (130 mg, 0.258 mmol) in DCM (5 mL) was added water (1.2 mL), followed by TFA (4 mL, 51.9 mmol). The reaction was stirred at room temperature overnight. The reaction mixture was then concentrated under reduced pressure. The residue was dissolved in a mixture of DMSO/NH₄OH, filtered, and subjected to mass-triggered reverse phase HPLC (ACN/water with 0.1% NH₄OH modifier) to afford (2R,3R,3aS,6S,6aR)-6-((2-amino-3-fluoroquinolin-7-yl)methyl)-2-(4-amino-7H-pyrrolo[2,3-d]pyrimidin-7-yl)-6a-methylhexahydro-3aH-cyclopenta[b]furan-3,3a-diol as a solid. MS: 465 (M+1). ¹H NMR (600 MHz, DMSO-d₆) δ 8.09 (s, 1H), 7.74 (d, J=11.8 Hz, 1H), 7.54 (d, J=8.2 Hz, 1H), 7.44 (d, J=3.7 Hz, 1H), 7.28 (s, 1H), 7.07 (d, J=8.5 Hz, 1H), 7.04 (s, 2H), 6.69-6.63 (m, 3H), 5.85 (d, J=8.2 Hz, 1H), 5.34 (d, J=7.0 Hz, 1H), 4.84 (s, 1H), 4.15 (t, J=7.6 Hz, 1H), 2.84 (dd, J=13.5, 4.8 Hz, 1H), 2.57-2.51 (m, 1H), 1.89 (dd, J=11.7, 5.5 Hz, 1H), 1.87-1.80 (m, 1H), 1.64-1.55 (m, 1H), 1.52-1.40 (m, 2H), 1.17 (s, 3H).

### Example 122

### (2R,3R,3aS,6S,6aR)-6-((2-amino-3-chloroquinolin-7-yl)methyl)-2-(4-methyl-7H-pyrrolo[2,3-d]pyrimidin-7-yl)hexahydro-3aH-cyclopenta[b]furan-3,3a-diol

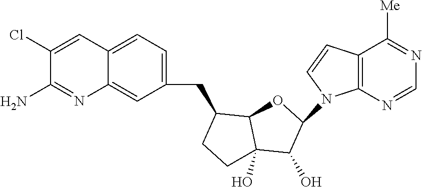 [SMILES: Cc1ncnc2c1ccn2[C@@H]1O[C@@H]2[C@H](Cc3ccc4cc(Cl)c(N)nc4c3)CC[C@]2(O)[C@H]1O]

Step 1: To a solution of (3aR,5aR,8aR)-4-methoxy-2,2-dimethyl-6-methylenehexahydrocyclopenta[2,3]furo[3,4-d][1,3]dioxole (123 mg, 0.544 mmol) in anhydrous THF (2 mL) was added 9-BBN solution (5.44 mL, 0.5M in THF, 2.72 mmol) dropwise at 0° C. under an argon atmosphere. The reaction mixture was then stirred at 52° C. for 1 h. The reaction mixture was cooled to room temperature. To the reaction mixture was added a solution of potassium phosphate tribasic (576 mg, 2.71 mmol) in water (0.2 mL) dropwise at 0° C. under argon. The reaction was stirred at room temperature for 0.5 h. 7-bromo-3-chloroquinolin-2-amine (140 mg, 0.543 mmol) in THF (0.3 mL) and 1,1′-bis(diphenylphosphino)ferrocene-palladium(II)dichloride dichloromethane complex (44.3 mg, 0.054 mmol) were added at room temperature. The reaction mixture was irradiated with microwave radiation at 70° C. for 2 h. The reaction was cooled to room temperature, diluted with water (15 mL), and extracted with EtOAc (2×20 mL). The organic layer was dried over anhydrous sodium sulfate and filtered. The filtrate was concentrated under reduced pressure, and the resulting residue was purified by prep-TLC (eluted with 20% EtOAc/PE) to afford 3-chloro-7-(((3aR,4S,5aR,8aR)-4-methoxy-2,2-dimethylhexahydrocyclopenta[2,3]furo[3,4-d][1,3]dioxol-6-yl)methyl)quinolin-2-amine as a solid. MS: 405 (M+1).

Step 2: A solution of 3-chloro-7-(((3aR,4S,5aR,8aR)-4-methoxy-2,2-dimethylhexahydrocyclopenta[2,3]furo[3,4-d][1,3]dioxol-6-yl)methyl)quinolin-2-amine (100 mg, 0.247 mmol) in water (6 mL) and acetonitrile (9 mL) was added concentrated HCl (0.5 mL, 6.00 mmol) at room temperature. The solution was stirred at 90° C. for 1 h. The reaction solution was cooled to 0° C. and then sodium bicarbonate (500 mg) was added portion wise. The reaction mixture was concentrated under reduced pressure. The residue was purified by column chromatography on silica (0-10% MeOH/DCM) to afford (3R,3aS,6aR)-6-((2-amino-3-chloroquinolin-7-yl)methyl)hexahydro-2H-cyclopenta[b]furan-2,3,3a-triol as a solid. MS: 351 (M+1).

Step 3: To a stirred solution of (3R,3aS,6aR)-6-((2-amino-3-chloroquinolin-7-yl)methyl)hexahydro-2H-cyclopenta[b]furan-2,3,3a-triol (60 mg, 0.171 mmol) in dry MeCN (1 mL) under argon was added (E)-diazene-1,2-diylbis(piperidin-1-ylmethanone) (64.7 mg, 0.257 mmol) in MeCN (0.5 mL) dropwise at 0° C. This was followed by the addition of tributylphosphine (0.068 mL, 0.274 mmol) in MeCN (0.5 mL) dropwise at 0° C. The resulting solution was stirred at 30° C. for ˜1 h. Separately, to a stirred solution of 4-methyl-7H-pyrrolo[2,3-d]pyrimidine (43.3 mg, 0.325 mmol) in dry DMF (1 mL) was added NaH (12.31 mg, 60% in mineral oil, 0.308 mmol) at room temperature. The suspension was stirred at room temperature for 30 minutes, then the suspension was transferred to the solution originally containing the triol via syringe. The resulting reaction mixture was stirred at room temperature for 1 h. The reaction was quenched with water (10 mL) and extracted with EtOAc (2×10 mL). The combined organic layers were dried over anhydrous sodium sulfate and filtered. The filtrate was concentrated under reduced pressure. The residue was purified by reverse phase column chromatography (ACN/water with 10 mM NH₄HCO₃ modifier) to afford (2R,3R,3aS,6S,6aR)-6-((2-amino-3-chloroquinolin-7-yl)methyl)-2-(4-methyl-7H-pyrrolo[2,3-d]pyrimidin-7-yl)hexahydro-2H-cyclopenta[b]furan-3,3a-diol as a solid. MS: 466 (M+1). ¹H-NMR (400 MHz, DMSO-d₆) δ 8.69 (s, 1H), 8.12 (s, 1H), 7.88 (d, J=3.6 Hz, 1H), 7.54 (d, J=8.0 Hz, 1H), 7.28 (s, 1H), 7.08 (dd, J=8.4, 1.6 Hz, 1H), 6.82 (d, J=3.6 Hz, 1H), 6.64 (br s, 2H), 6.00 (d, J=8.4 Hz, 1H), 5.31 (d, J=6.8 Hz, 1H), 5.12 (s, 1H), 4.22 (t, J=8.0 Hz, 1H), 4.00 (d, J=6.0 Hz, 1H), 2.85-2.79 (m, 1H), 2.69 (s, 3H), 2.65-2.60 (m, 1H), 2.33-2.25 (m, 1H), 1.98-1.93 (m, 1H), 1.80-1.67 (m, 2H), 1.58-1.51 (m, 1H).

### Example 123

### (2R,3R,3aS,6S,6aR)-6-((2-amino-3-fluoroquinolin-7-yl)methyl)-2-(4-methyl-7H-pyrrolo[2,3-d]pyrimidin-7-yl)hexahydro-3aH-cyclopenta[b]furan-3,3a-diol

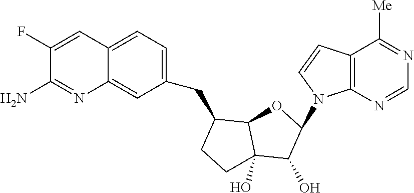 [SMILES: Cc1ncnc2c1ccn2[C@@H]1O[C@@H]2[C@H](Cc3ccc4cc(F)c(N)nc4c3)CC[C@]2(O)[C@H]1O]

Step 1: To a solution of (3aR,5aR,8aR)-4-methoxy-2,2-dimethyl-6-methylenehexahydrocyclopenta[2,3]furo[3,4-d][1,3]dioxole (245 mg, 1.08 mmol) in anhydrous THF (2.5 mL) was added 9-BBN solution (8.66 mL, 0.5M in THF, 4.33 mmol) dropwise at 0° C. under argon. The reaction solution was stirred at 55° C. for 1 h. To this reaction solution was added a solution of K₃PO₄ (1.15 g, 5.40 mmol) in water (2 mL) at 0° C. under argon. The reaction was stirred at room temperature for 0.5 h. Then a solution of 7-bromo-3-fluoroquinolin-2-amine (273 mg, 1.13 mmol) in THF (2 mL) and Pd(dppf)Cl₂ (119 mg, 0.162 mmol) were added at room temperature. The reaction mixture was stirred at 75° C. for 1.5 h. The reaction was concentrated under reduced pressure, and the residue was purified by preparative TLC (2:1 EtOAc/PE) to afford 3-fluoro-7-(((3aR,4S,5aR,8aR)-4-methoxy-2,2-dimethylhexahydrocyclopenta[2,3]furo[3,4-d][1,3]dioxol-6-yl)methyl)quinolin-2-amine as a solid. MS: 389 (M+1).

Step 2: A solution of 3-fluoro-7-(((3aR,4S,5aR,8aR)-4-methoxy-2,2-dimethylhexahydrocyclopenta[2,3]furo[3,4-d][1,3]dioxol-6-yl)methyl)quinolin-2-amine (300 mg, 0.772 mmol) in 0.4M aqueous HCl in MeCN/H₂O (3:2) (6 mL, 2.400 mmol) was stirred at 90° C. for 1 h. The reaction solution was then cooled to 0° C., and quenched with saturated aqueous Na₂CO₃ (60 mL) and extracted with EtOAc (100 mL×3). The combined organic layers were washed with brine (60 mL), dried over anhydrous sodium sulfate, filtered, and the filtrate was concentrated under reduced pressure. The resulting residue was purified by column chromatography on silica (0-10% MeOH/DCM) to afford (3R,3aS,6aR)-6-((2-amino-3-fluoroquinolin-7-yl)methyl)hexahydro-2H-cyclopenta[b]furan-2,3,3a-triol as a solid. MS: 335 (M+1).

Step 3: To a stirred solution of (3R,3aS,6aR)-6-((2-amino-3-fluoroquinolin-7-yl)methyl)hexahydro-2H-cyclopenta[b]furan-2,3,3a-triol (0.067 g, 0.2 mmol) in dry MeCN (3 mL) was added tributylphosphine (0.077 g, 0.38 mmol), followed by (E)-diazene-1,2-diylbis(piperidin-1-ylmethanone) (0.091 g, 0.36 mmol) at room temperature. The reaction mixture was stirred at room temperature for 1 h. Separately, to a stirred solution of 4-methyl-7H-pyrrolo[2,3-d]pyrimidine (0.053 g, 0.400 mmol) in dry DMF (2 mL) was added NaH (0.024 g, 60% in mineral oil, 0.600 mmol) at 0° C. The suspension was stirred at room temperature for 30 minutes. The suspension was then transferred to the solution originally containing the triol via syringe. The resulting reaction was stirred at room temperature for 2 h. The reaction mixture was then quenched with saturated ammonium chloride (30 mL) and extracted with EtOAc (40 mL×3). The combined organic layers were washed with brine (40 mL), dried over anhydrous sodium sulfate, and filtered. The filtrate was concentrated under reduced pressure, and the resulting residue was purified by preparative TLC (1:1 DCM/MeOH). The product was further purified by reverse phase column chromatography (ACN/water with 5 mM NH₄HCO₃ modifier) to afford (2R,3R,3aS,6S,6aR)-6-((2-amino-3-fluoroquinolin-7-yl)methyl)-2-(4-methyl-7H-pyrrolo[2,3-d]pyrimidin-7-yl)hexahydro-2H-cyclopenta[b]furan-3,3a-diol as a solid. MS: 450 (M+1). ¹H-NMR (400 MHz, DMSO-d₆) δ 8.69 (s, 1H), 7.87 (d, J=4.0 Hz, 1H), 7.74 (d, J=11.6 Hz, 1H), 7.52 (d, J=8.0 Hz, 1H), 7.28 (s, 1H), 7.07 (d, J=8.0 Hz, 1H), 6.82 (d, J=3.6 Hz, 1H), 6.66 (br s, 2H), 6.01 (d, J=8.0 Hz, 1H), 5.31 (d, J=7.2 Hz, 1H), 5.12 (s, 1H), 4.22 (d, J=7.6 Hz, 1H), 4.01 (d, J=6.0 Hz, 1H), 2.84-2.79 (m, 1H), 2.69 (s, 3H), 2.67-2.59 (m, 1H), 2.28-2.22 (m, 1H), 1.98-1.94 (m, 1H), 1.76-1.69 (m, 2H), 1.58-1.53 (m, 1H).

### Example 124

### (2R,3R,3aS,6S,6aR)-6-((2-amino-3-fluoroquinolin-7-yl)methyl)-2-(4-amino-7H-pyrrolo[2,3-d]pyrimidin-7-yl)hexahydro-3aH-cyclopenta[b]furan-3,3a-diol

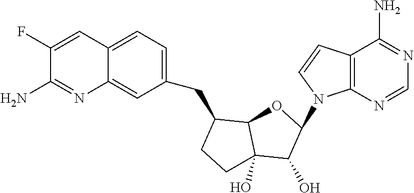 [SMILES: Nc1nc2cc(C[C@@H]3CC[C@@]4(O)[C@@H]3O[C@@H](n3ccc5c(N)ncnc53)[C@@H]4O)ccc2cc1F]

Step 1: To a stirred solution of (3R,3aS,6aR)-6-((2-amino-3-fluoroquinolin-7-yl)methyl)hexahydro-2H-cyclopenta[b]furan-2,3,3a-triol (0.100 g, 0.3 mmol) in dry MeCN (4.5 mL) was added tributylphosphine (0.115 g, 0.57 mmol), followed by (E)-diazene-1,2-diylbis(piperidin-1-ylmethanone) (0.136 g, 0.54 mmol) at room temperature. The reaction mixture was stirred at room temperature for 1 h. Separately, to a stirred solution of 4-chloro-7H-pyrrolo[2,3-d]pyrimidine (0.092 g, 0.60 mmol) in dry DMF (2 mL) was added sodium hydride (0.036 g, 60% in mineral oil, 0.90 mmol) at 0° C. The suspension was stirred at room temperature for 30 minutes. The suspension was then transferred to the solution originally containing the triol via syringe. The resulting reaction was stirred at room temperature for 2 h. The reaction mixture was quenched with saturated ammonium chloride (40 mL) and extracted with EtOAc (100 mL×3). The combined organic layers were washed with brine (40 mL), dried over anhydrous sodium sulfate, and filtered. The filtrate was concentrated under reduced pressure, and the resulting residue was purified by preparative TLC (1:1 PE/EtOAc) to afford (2R,3R,3aS,6aR)-6-((2-amino-3-fluoroquinolin-7-yl)methyl)-2-(4-chloro-7H-pyrrolo[2,3-d]pyrimidin-7-yl)hexahydro-2H-cyclopenta[b]furan-3,3a-diol as solid. MS: 470 (M+1).

Step 2: To a mixture of (2R,3R,3aS,6aR)-6-((2-amino-3-fluoroquinolin-7-yl)methyl)-2-(4-chloro-7H-pyrrolo[2,3-d]pyrimidin-7-yl)hexahydro-2H-cyclopenta[b]furan-3,3a-diol (70 mg, 0.149 mmol) in dioxane (8 mL) was added ammonia hydrate (8 mL, 28%, 0.050 mmol) in a sealed tube at room temperature. Then the reaction mixture was heated at 95° C. for 16 h. The reaction was concentrated under reduced pressure, and the residue was purified by preparative TLC (10:1 DCM/MeOH). The product was further purified by reverse phase column chromatography (ACN/water with 5 mM NH₄HCO₃ modifier) to afford (2R,3R,3aS,6S,6aR)-6-((2-amino-3-fluoroquinolin-7-yl)methyl)-2-(4-amino-7H-pyrrolo[2,3-d]pyrimidin-7-yl)hexahydro-2H-cyclopenta[b]furan-3,3a-diol as a solid. MS: 451 (M+1). ¹H-NMR (400 MHz, DMSO-d₆) δ 8.09 (s, 1H), 7.74 (d, J=12.0 Hz, 1H), 7.53 (d, J=8.4 Hz, 1H), 7.44 (d, J=3.6 Hz, 1H), 7.29 (s, 1H), 7.09-7.05 (m, 3H), 6.68-6.66 (m, 3H), 5.88 (d, J=8.4 Hz, 1H), 5.25-5.20 (m, 1H), 5.07-5.05 (m, 1H), 4.13 (d, J=8.0 Hz, 1H), 3.95 (d, J=5.6 Hz, 1H), 2.84-2.79 (m, 1H), 2.67-2.58 (m, 1H), 2.29-2.13 (m, 1H), 1.96-1.92 (m, 1H), 1.74-1.68 (m, 2H), 1.57-1.49 (m, 1H).

### Example 125

### (2R,3R,3aS,6S,6aR)-6-((2-amino-3-methylquinolin-7-yl)methyl)-2-(4-amino-7H-pyrrolo[2,3-d]pyrimidin-7-yl)hexahydro-3aH-cyclopenta[b]furan-3,3a-diol

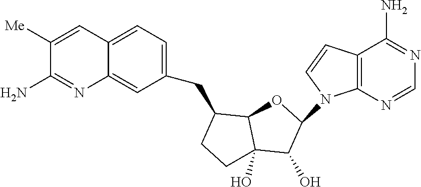 [SMILES: Cc1cc2ccc(C[C@@H]3CC[C@@]4(O)[C@@H]3O[C@@H](n3ccc5c(N)ncnc53)[C@@H]4O)cc2nc1N]

Step 1: Into a microwave tube were added (2R,3R,3aS,6S,6aR)-6-((2-amino-3-bromoquinolin-7-yl)methyl)-2-(4-amino-7H-pyrrolo[2,3-d]pyrimidin-7-yl)hexahydro-2H-cyclopenta[b]furan-3,3a-diol (40 mg, 0.078 mmol), 2,4,6-trimethyl-1,3,5,2,4,6-trioxatriborinane (19.6 mg, 0.156 mmol), PdCl₂(dppf) (14.4 mg, 0.020 mmol), K₂CO₃ in water (0.96 mL, 2M, 1.92 mmol), and DMF (2 mL) at room temperature under argon. The reaction mixture was irradiated with microwave radiation for 1 h at 130° C. The reaction was cooled to room temperature and concentrated under reduced pressure. The resulting residue was purified by reverse phase column chromatography (ACN/water with 5 mM NH₄HCO₃ modifier). The product was further purified by Prep-HPLC (ACN/water with 10 mM NH₄HCO₃ modifier) to afford (2R,3R,3aS,6S,6aR)-6-((2-amino-3-methylquinolin-7-yl)methyl)-2-(4-amino-7H-pyrrolo[2,3-d]pyrimidin-7-yl)hexahydro-2H-cyclopenta[b]furan-3,3a-diol as a solid. MS: 447 (M+1). ¹H-NMR (400 MHz, DMSO-d₆) δ 8.10 (s, 1H), 7.65 (s, 1H), 7.47-7.45 (m, 2H), 7.25 (s, 1H), 7.06 (br s, 2H), 7.00 (dd, J=8.0, 1.2 Hz, 1H), 6.68 (d, J=4.0 Hz, 1H), 6.16 (br s, 2H), 5.90 (d, J=8.4 Hz, 1H), 5.25 (d, J=7.2 Hz, 1H), 5.07 (s, 1H), 4.14 (t, J=7.6 Hz, 1H), 3.97 (d, J=5.6 Hz, 1H), 2.84-2.79 (m, 1H), 2.63-2.58 (m, 1H), 2.28-2.22 (m, 1H), 2.19 (s, 3H), 1.95 (dd, J=12.0, 4.4 Hz, 1H), 1.75-1.70 (m, 2H), 1.56-1.51 (m, 1H).

### Example 126

### (1S,2R,3S,5R)-5-(4-amino-7H-pyrrolo[2,3-d]pyrimidin-7-yl)-3-(2-(2,3-dihydro-1H-pyrrolo[2,3-b]quinolin-7-yl)ethyl)-3-methylcyclopentane-1,2-diol

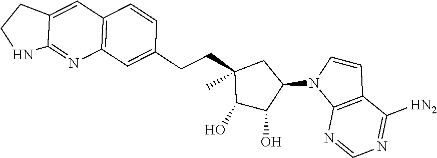

Step 1: To a solution of 7-((3a′R,4′R,6′R,6a'S)-4′-methyl-4′-vinyltetrahydro-4′H-spiro[cyclohexane-1,2′-cyclopenta[d][1,3]dioxol]-6′-yl)-7H-pyrrolo[2,3-d]pyrimidin-4-amine (223 mg, 0.63 mmol) in THF (1 mL) was added 9-BBN solution (3.16 mL, 0.5 M in THF, 1.58 mmol) under an atmosphere of nitrogen, and mixture was heated to 50° C. for a few hours, and then cooled to room temperature and quenched with K₃PO₄ (˜3 mL, 2 M in water). The mixture was stirred for 15 mins, and the organic layer was separated from the aqueous layer. To a mixture of RuPhos-Pd-G3 (12.4 mg, 0.015 mmol) and tert-butyl 7-bromo-2,3-dihydro-1H-pyrrolo[2,3-b]quinoline-1-carboxylate (56.9 mg, 0.16 mmol) was added THF (0.4 mL) under an atmosphere of nitrogen. Next, one third of the aforementioned organic layer of 7-((3a′R,4'S,6′R,6a'S)-4′-(2-((1R,5R)-9-borabicyclo[3.3.1]nonan-9-yl)ethyl)-4′-methyltetrahydro-3a′H-spiro[cyclohexane-1,2′-cyclopenta[d][1,3]dioxol]-6′-yl)-7H-pyrrolo[2,3-d]pyrimidin-4-amine solution (1.39 mL, 0.15 mmol) and K₃PO₄ (0.37 mL, 2 M in water, 0.74 mmol) was injected simultaneously. An additional rinse of the borane solution vial was added to the reaction vial with THF (0.3 mL). The resulting mixture was heated to 50° C. overnight, and then cooled to room temperature. The reaction mixture was diluted with DCM and water. The organic and aqueous layers were separated. The combined organic layers were concentrated under reduced pressure. The residue was re-dissolved in THF (0.5 mL), water (0.5 mL, 27.8 mmol), and TFA (0.5 mL, 6.5 mmol), and the resulting mixture was heated to 50° C. for a couple hours, and then cooled to room temperature, and concentrated under reduced pressure. The residue was purified by mass-triggered reverse phase HPLC (MeCN/H₂O with 0.1% NH₄OH modifier) to afford (1S,2R,3S,5R)-5-(4-amino-7H-pyrrolo[2,3-d]pyrimidin-7-yl)-3-(2-(2,3-dihydro-1H-pyrrolo[2,3-b]quinolin-7-yl)ethyl)-3-methylcyclopentane-1,2-diol as a solid. MS: 445 (M+1). ¹H NMR (600 MHz, DMSO-d₆) δ 8.05 (s, 1H), 7.61 (s, 1H), 7.47 (d, J=8.1 Hz, 1H), 7.30-7.24 (m, 2H), 7.13 (s, 1H), 7.09-6.98 (m, 1H), 6.92 (s, 2H), 6.54 (d, J=3.5 Hz, 1H), 4.93-4.83 (m, 2H), 4.59 (d, J=5.5 Hz, 1H), 4.40 (q, J=6.2 Hz, 1H), 3.78 (t, J=5.8 Hz, 1H), 3.57 (t, J=7.8 Hz, 2H), 3.10 (t, J=7.5 Hz, 2H), 2.72 (td, J=12.9, 5.8 Hz, 1H), 2.62 (td, J=13.3, 12.9, 5.7 Hz, 1H), 1.88 (dd, J=12.8, 8.8 Hz, 1H), 1.84-1.70 (m, 3H), 1.11 (s, 3H).

### Example 127

### (2R,3R,3aS,6S,6aR)-6-((2-amino-3-bromoquinolin-7-yl)methyl)-2-(2-amino-4-methyl-7H-pyrrolo[2,3-d]pyrimidin-7-yl)hexahydro-3aH-cyclopenta[b]furan-3,3a-diol

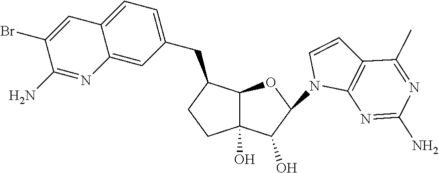 [SMILES: Cc1nc(N)nc2c1ccn2[C@@H]1O[C@@H]2[C@H](Cc3ccc4cc(Br)c(N)nc4c3)CC[C@]2(O)[C@H]1O]

Step 1: To an oven-dried, argon-cooled 2-5 mL microwave vial containing (3R,3aS,6S,6aR)-6-[(2-amino-3-bromoquinolin-7-yl)methyl]hexahydro-3aH-cyclopenta[b]furan-2,3,3a-triol (70 mg, 0.177 mmol) dissolved in dry MeCN (3 mL) was added 1,1′-(azodicarbonyl)dipiperidine (67.0 mg, 0.266 mmol) followed by tri-n-butylphosphine (70.8 μl, 0.283 mmol) at room temperature. The mixture was stirred for 1 h. In a separate oven-dried, argon-cooled vial containing di-tert-butyl (4-methyl-7H-pyrrolo[2,3-d]pyrimidin-2-yl)imidodicarbonate (93 mg, 0.266 mmol) dissolved in anhydrous acetonitrile (1 mL) was added DBU (53.4 μl, 0.354 mmol). The mixture was stirred at room temperature for 30 minutes, and then this suspension was transferred to the mixture described above originally containing the triol via syringe. After 30 minutes at room temperature, the reaction was heated to 40° C. for ˜4 h. The mixture was then cooled to room temperature and quenched with water and extracted with EtOAc (3×). The combined organics were then washed with brine, dried over sodium sulfate, filtered, and concentrated under reduced pressure. The residue was purified by column chromatography on silica (0-100% EtOAc:hexanes) to afford di-tert-butyl (7-{(2R,3R,3aS,6S,6aR)-6-[(2-amino-3-bromoquinolin-7-yl)methyl]-3,3a-dihydroxyhexahydro-2H-cyclopenta[b]furan-2-yl}-4-methyl-7H-pyrrolo[2,3-d]pyrimidin-2-yl)imidodicarbonate as a solid. MS: 725/727 (M+1/M+3).

Step 2: To a solution of di-tert-butyl (7-{(2R,3R,3aS,6S,6aR)-6-[(2-amino-3-bromoquinolin-7-yl)methyl]-3,3a-dihydroxyhexahydro-2H-cyclopenta[b]furan-2-yl}-4-methyl-7H-pyrrolo[2,3-d]pyrimidin-2-yl)imidodicarbonate (39.8 mg, 0.055 mmol) in DCM (1.10 mL) was added TFA 20 equivalents), and the reaction was stirred for overnight at room temperature. The mixture was then concentrated under reduced pressure, and the residue was purified by mass triggered reverse phase HPLC (MeCN:H₂O gradient with 0.1% NH₄OH) to afford (2R,3R,3aS,6S,6aR)-6-((2-amino-3-bromoquinolin-7-yl)methyl)-2-(2-amino-4-methyl-7H-pyrrolo[2,3-d]pyrimidin-7-yl)hexahydro-3aH-cyclopenta[b]furan-3,3a-diol as a solid. MS: 525/527 (M+1/M+3). ¹H NMR (600 MHz, DMSO-d₆) δ 8.31 (s, 1H), 7.55 (d, J=8.2 Hz, 1H), 7.32 (d, J=3.8 Hz, 1H), 7.28 (s, 1H), 7.09 (dd, J=8.2, 1.4 Hz, 1H), 6.55 (s, 2H), 6.51 (d, J=3.8 Hz, 1H), 6.18 (s, 2H), 5.82 (d, J=8.2 Hz, 1H), 5.27 (d, J=6.9 Hz, 1H), 5.03 (s, 1H), 4.08-4.03 (m, 1H), 3.91 (d, J=5.7 Hz, 1H), 2.83-2.78 (m, 1H), 2.64-2.59 (m, 1H), 2.45 (s, 3H), 2.28-2.21 (m, 1H), 1.97-1.93 (m, 1H), 1.72-1.67 (m, 2H), 1.57-1.49 (m, 1H).

### Example 128

### (2R,3R,3aS,6S,6aR)-6-((2-amino-3-(difluoromethyl)quinolin-7-yl)oxy)-2-(4-amino-7H-pyrrolo[2,3-d]pyrimidin-7-yl)hexahydro-3aH-cyclopenta[b]furan-3,3a-diol

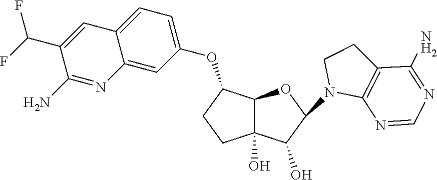 [SMILES: Nc1nc2cc(O[C@H]3CC[C@@]4(O)[C@@H]3O[C@@H](N3CCc5c(N)ncnc53)[C@@H]4O)ccc2cc1C(F)F]

Step 1: To a stirred solution of (3R,3aS,6R,6aR)-6-(benzyloxy)hexahydro-2H-cyclopenta[b]furan-2,3,3a-triol (3.4 g, 12.8 mmol) in anhydrous MeCN (170 mL) was added tributylphosphine (5.10 mL, 20.4 mmol) in MeCN (70 mL) dropwise at 0° C. under argon. This was followed by the addition of (E)-diazene-1,2-diylbis(piperidin-1-ylmethanone) (4.83 g, 19.2 mmol) in MeCN (70 mL) dropwise at 0° C. under argon. The resulting mixture was stirred at 40° C. for 30 minutes. Separately, to a stirred solution of 4-chloro-7H-pyrrolo[2,3-d]pyrimidine (3.67 g, 23.88 mmol) in dry MeCN (35 mL) was added DBU (3.62 mL, 24.01 mmol) dropwise at 0° C. under argon. The resulting solution was stirred at 30° C. for 1 h. Then this solution was transferred to the solution originally containing the triol via syringe over 1 minute. The resulting reaction was stirred at 39° C. for 2 h. The reaction was diluted with EtOAc (200 mL), washed with water (2×100 mL), and washed with brine (100 mL). The organic layer was dried over anhydrous sodium sulfate and filtered. The filtrate was concentrated under reduced pressure, and the residue was purified by reverse phase HPLC (ACN/water with 0.05% NH₄HCO₃) to afford (2R,3R,3aS,6R,6aR)-6-(benzyloxy)-2-(4-chloro-7H-pyrrolo[2,3-d]pyrimidin-7-yl)hexahydro-2H-cyclopenta[b]furan-3,3a-diol as a solid. MS: 402 (M+1).

Step 2: To a solution of (2R,3R,3aS,6R,6aR)-6-(benzyloxy)-2-(4-chloro-7H-pyrrolo[2,3-d]pyrimidin-7-yl)hexahydro-2H-cyclopenta[b]furan-3,3a-diol (8.6 g, 15.2 mmol, 71 wt %) in 2,2-dimethoxypropane (100 mL) was added 4-methylbenzenesulfonic acid (0.262 g, 1.52 mmol) at room temperature under argon. The mixture was stirred at 70° C. for 16 h. The reaction was cooled to room temperature and quenched with saturated aqueous sodium bicarbonate (10 mL). The mixture was concentrated under reduced pressure, and the resulting residue was purified by column chromatography on silica (0-50% EtOAc/PE) to afford 7-((3aR,4R,5aR,6R,8aR)-6-(benzyloxy)-2,2-dimethylhexahydrocyclopenta[2,3]furo[3,4-d][1,3]dioxol-4-yl)-4-chloro-7H-pyrrolo[2,3-d]pyrimidine as an oil. MS: 442 (M+1).

Step 3: To a solution of 7-((3aR,4R,5aR,6R,8aR)-6-(benzyloxy)-2,2-dimethylhexahydrocyclopenta[2,3]furo[3,4-d][1,3]dioxol-4-yl)-4-chloro-7H-pyrrolo[2,3-d]pyrimidine (5 g, 10.75 mmol) in dioxane (60 mL) was added ammonium hydroxide (120 mL, 28%, 872 mmol) at room temperature. The mixture was sealed tightly and stirred at 90° C. for 16 h. The reaction mixture was concentrated under reduced pressure, and the resulting residue was purified by column chromatography on silica (0-10% MeOH/DCM) to afford 7-((3aR,4R,5aR,6R,8aR)-6-(benzyloxy)-2,2-dimethylhexahydrocyclopenta[2,3]furo[3,4-d][1,3]dioxol-4-yl)-7H-pyrrolo[2,3-d]pyrimidin-4-amine as a solid. MS: 423 (M+1).

Step 4: To a mixture of 7-((3aR,4R,5aR,6R,8aR)-6-(benzyloxy)-2,2-dimethylhexahydrocyclopenta[2,3]furo[3,4-d][1,3]dioxol-4-yl)-7H-pyrrolo[2,3-d]pyrimidin-4-amine (339 mg, 0.722 mmol) in MeOH (40 mL) was added palladium hydroxide on carbon (1.78 g, 20%, 50% in water, 2.53 mmol) at room temperature under argon. The suspension was degassed under vacuum and purged with H₂ several times. The reaction was stirred under 2 atm of H₂ at room temperature for 2 h. The mixture was filtered through a Celite pad, and the filtrate was concentrated under reduced pressure to afford (3aR,4R,5aR,6R,8aR)-4-(4-amino-7H-pyrrolo[2,3-d]pyrimidin-7-yl)-2,2-dimethylhexahydrocyclopenta[2,3]furo[3,4-d][1,3]dioxol-6-ol as a solid. MS: 333 (M+1).

Step 5: To a stirred mixture of (3aR,4R,5aR,6R,8aR)-4-(4-amino-7H-pyrrolo[2,3-d]pyrimidin-7-yl)-2,2-dimethylhexahydrocyclopenta[2,3]furo[3,4-d][1,3]dioxol-6-ol (170 mg, 0.512 mmol) in DCM (5 mL) and Pyridine (0.5 mL) was added trifluoromethanesulfonic anhydride (188 mg, 0.665 mmol) at 0° C. under argon. The resulting mixture was stirred for 2 h at 0° C. The reaction mixture was then quenched with saturated NaHCO₃ (30 mL), extracted with EtOAc (30 mL×3), and the combined organic layers were dried over anhydrous sodium sulfate, filtered, and the filtrate was concentrated under reduced pressure. The resulting residue was purified by Prep-TLC (1:1 PE: EtOAc) to afford (3aR,4R,5aR,6R,8aR)-4-(4-amino-7H-pyrrolo[2,3-d]pyrimidin-7-yl)-2,2-dimethylhexahydrocyclopenta[2,3]furo[3,4-d][1,3]dioxol-6-yl trifluoromethanesulfonate as a solid. MS: 465 (M+1).

Step 6: To a stirred solution of (3aR,4R,5aR,6R,8aR)-4-(4-amino-7H-pyrrolo[2,3-d]pyrimidin-7-yl)-2,2-dimethylhexahydrocyclopenta[2,3]furo[3,4-d][1,3]dioxol-6-yl trifluoromethanesulfonate (90 mg, 0.194 mmol) and 2-amino-3-(difluoromethyl)quinolin-7-ol (44.8 mg, 0.213 mmol) in NMP (0.5 mL) was added Cs₂CO₃ (95 mg, 0.291 mmol) at 25° C. under argon. The resulting mixture was stirred for 2 h at 25° C. The reaction mixture was purified by reverse phase column chromatography (ACN/water with 5 mM NH₄HCO₃ modifier) to afford 7-(((3aR,4R,5aR,6S,8aR)-4-(4-amino-7H-pyrrolo[2,3-d]pyrimidin-7-yl)-2,2-dimethylhexahydrocyclopenta[2,3]furo[3,4-d][1,3]dioxol-6-yl)oxy)-3-(difluoromethyl)quinolin-2-amine as a solid. MS: 525 (M+1).

Step 7: To the vial charged with 7-(((3aR,4R,5aR,6S,8aR)-4-(4-amino-7H-pyrrolo[2,3-d]pyrimidin-7-yl)-2,2-dimethylhexahydrocyclopenta[2,3]furo[3,4-d][1,3]dioxol-6-yl)oxy)-3-(difluoromethyl)quinolin-2-amine (50 mg, 0.095 mmol) were added TFA (1 mL) and water (1 mL) at 25° C. The resulting mixture was stirred at 25° C. for 16 h. The reaction mixture was concentrated under reduced pressure, and the residue was purified by reverse phase column chromatography (ACN/water with 5 mM NH₄HCO₃ modifier) to afford (2R,3R,3aS,6S,6aR)-6-((2-amino-3-(difluoromethyl)quinolin-7-yl)oxy)-2-(4-amino-7H-pyrrolo[2,3-d]pyrimidin-7-yl)hexahydro-2H-cyclopenta[b]furan-3,3a-diol as a solid. MS: 485 (M+1). ¹H-NMR (400 MHz, CD₃OD) δ 8.10-8.09 (m, 2H), 7.65 (d, J=8.8 Hz, 1H), 7.41 (d, J=3.6 Hz, 1H), 7.00-6.95 (m, 2H), 6.86-6.63 (m, 2H), 6.04 (d, J=8.4 Hz, 1H), 4.77 (d, J=4.8 Hz, 1H), 4.49 (d, J=8.4 Hz, 1H), 4.30 (s, 1H), 2.53-2.43 (m, 1H), 2.26-2.16 (m, 3H).

### Example 129

### (2R,3R,3aS,6R,6aR)-6-((2-amino-3-bromoquinolin-7-yl)oxy)-2-(4-methyl-7H-pyrrolo[2,3-d]pyrimidin-7-yl)hexahydro-3aH-cyclopenta[b]furan-3,3a-diol

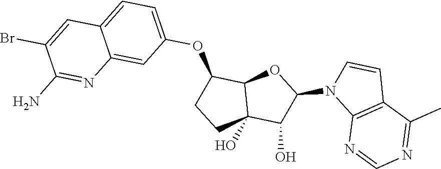 [SMILES: Cc1ncnc2c1ccn2[C@@H]1O[C@@H]2[C@H](Oc3ccc4cc(Br)c(N)nc4c3)CC[C@]2(O)[C@H]1O]

Step 1: To a vial charged with (3aR,4R,5aR,6R,8aR)-2,2-dimethyl-4-(4-methyl-7H-pyrrolo[2,3-d]pyrimidin-7-yl)hexahydrocyclopenta[2,3]furo[3,4-d][1,3]dioxol-6-ol (50 mg, 0.15 mmol), 3-bromo-N-(2,4-dimethoxybenzyl)-7-iodoquinolin-2-amine (113 mg, 0.23 mmol), 4-(pyrrolidin-1-yl)pyridine (26.8 mg, 0.181 mmol), copper iodide (2.87 mg, 0.015 mmol), potassium phosphate tribasic (128 mg, 0.60 mmol) was added toluene (754 μl), and the reaction was heated to 120° C. overnight. The reaction was filtered, concentrated under reduced pressure, and the residue was purified by column chromatography on silica (10-100% EtOAc/CH₂Cl₂) to afford 3-bromo-N-(2,4-dimethoxybenzyl)-7-(((3aR,4R,5aR,6R,8aR)-2,2-dimethyl-4-(4-methyl-7H-pyrrolo[2,3-d]pyrimidin-7-yl)hexahydrocyclopenta[2,3]furo[3,4-d][1,3]dioxol-6-yl)oxy)quinolin-2-amine as a solid. MS: 702/704 (M+1/M+3).

Step 2: To a solution of 3-bromo-N-(2,4-dimethoxybenzyl)-7-(((3aR,4R,5aR,6R,8aR)-2,2-dimethyl-4-(4-methyl-7H-pyrrolo[2,3-d]pyrimidin-7-yl)hexahydrocyclopenta[2,3]furo[3,4-d][1,3]dioxol-6-yl)oxy)quinolin-2-amine (64 mg, 0.091 mmol) in CH₂Cl₂ (911 μl), was added TFA (1.40 mL, 18.2 mmol) and 1 drop of water. The reaction mixture was heated to 50° C. for 4 h. The solution was concentrated under reduced pressure, and the residue was purified by reverse phase HPLC (MeCN/water with 0.1% TFA modifier) to afford (2R,3R,3aS,6R,6aR)-6-((2-amino-3-bromoquinolin-7-yl)oxy)-2-(4-methyl-7H-pyrrolo[2,3-d]pyrimidin-7-yl)hexahydro-3aH-cyclopenta[b]furan-3,3a-diol as a solid. MS: 512/514 (M+1/M+3). ¹H NMR (500 MHz, DMSO-d₆) δ 8.63 (s, 1H), 8.24 (s, 1H), 7.78 (d, J=3.8 Hz, 1H), 7.51 (d, J=8.9 Hz, 1H), 6.94 (d, J=2.2 Hz, 1H), 6.86 (dd, J=8.8, 2.4 Hz, 1H), 6.77 (d, J=3.7 Hz, 1H), 6.54 (s, 2H), 6.14 (d, J=8.2 Hz, 1H), 5.46 (d, J=7.0 Hz, 1H), 5.39 (s, 1H), 4.84 (dt, J=8.8, 5.8 Hz, 1H), 4.41 (d, J=5.3 Hz, 1H), 4.33 (t, J=7.6 Hz, 1H), 2.64 (s, 3H), 2.25 (d, J=4.1 Hz, 1H), 2.22-2.12 (m, 1H), 2.11-2.03 (m, 1H), 1.73-1.64 (m, 1H).

### Example 130

### (2R,3R,3aS,6S,6aR)-6-((2-amino-3-bromoquinolin-7-yl)oxy)-2-(2-amino-5-fluoro-7H-pyrrolo[2,3-d]pyrimidin-7-yl)hexahydro-3aH-cyclopenta[b]furan-3,3a-diol

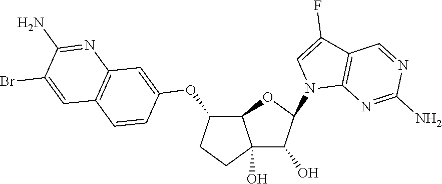 [SMILES: Nc1ncc2c(F)cn([C@@H]3O[C@@H]4[C@@H](Oc5ccc6cc(Br)c(N)nc6c5)CC[C@]4(O)[C@H]3O)c2n1]

Step 1: To a stirred slurry of 2-chloro-5-fluoro-7H-pyrrolo[2,3-d]pyrimidine (43.2 mg, 0.252 mmol) in THF (8.39 mL) was added pyridine (20.4 μl, 0.252 mmol), DIAD (103 μl, 0.529 mmol), and tri-n-butylphosphine (126 μl, 0.503 mmol). To the mixture was added (3R,3aS,6S,6aR)-6-((2-amino-3-bromoquinolin-7-yl)oxy)hexahydro-3aH-cyclopenta[b]furan-2,3,3a-triol (100 mg, 0.252 mmol) all at once. The mixture was left to stir for 2 h. The mixture was diluted with water and extracted with EtOAc (3×). The combined organics were washed with brine, dried over sodium sulfate, and concentrated under reduced pressure. The residue was purified by column chromatography on silica (0-20% MeOH/DCM) to afford (2R,3R,3aS,6S,6aR)-6-((2-amino-3-bromoquinolin-7-yl)oxy)-2-(2-chloro-5-fluoro-7H-pyrrolo[2,3-d]pyrimidin-7-yl)hexahydro-3aH-cyclopenta[b]furan-3,3a-diol. MS: 550/552 (M+1/M+3) Step 2: A mixture of (2R,3R,3aS,6S,6aR)-6-((2-amino-3-bromoquinolin-7-yl)oxy)-2-(2-chloro-5-fluoro-7H-pyrrolo[2,3-d]pyrimidin-7-yl)hexahydro-3aH-cyclopenta[b]furan-3,3a-diol (55.1 mg, 0.1 mmol) in 1,4-Dioxane (1 mL) and ammonium hydroxide (1 mL, 7.19 mmol) was irradiated under microwave to 100° C. for 2 h. The mixture was concentrated under reduced pressure, and the residue was purified by column chromatography on silica (0-15% MeOH/DCM). The product was further purified by mass triggered reverse phase HPLC (MeCN/water with 0.1% TFA modifier) to afford (2R,3R,3aS,6S,6aR)-6-((2-amino-3-bromoquinolin-7-yl)oxy)-2-(2-amino-5-fluoro-7H-pyrrolo[2,3-d]pyrimidin-7-yl)hexahydro-3aH-cyclopenta[b]furan-3,3a-diol as a solid. MS: 531/533 (M+1/M+3). ¹H NMR (500 MHz, DMSO-d₆) δ 8.68 (s, 1H), 8.63 (s, 1H), 7.74 (d, J=8.8 Hz, 1H), 7.59 (s, 1H), 7.25-6.72 (m, 5H), 5.99 (d, J=8.5 Hz, 1H), 4.63 (d, J=5.0 Hz, 1H), 4.23 (d, J=8.5 Hz, 2H), 4.03 (s, 2H), 2.13-1.90 (m, 3H).

### Example 131

### (2R,3R,3aS,6R,6aR)-6-((2-amino-3-chloroquinolin-7-yl)methyl)-2-(4-methyl-7H-pyrrolo[2,3-d]pyrimidin-7-yl)hexahydro-3aH-cyclopenta[b]furan-3,3a-diol

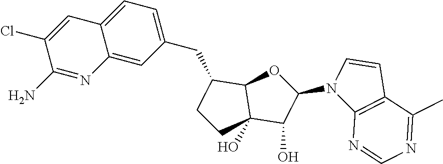 [SMILES: Cc1ncnc2c1ccn2[C@@H]1O[C@@H]2[C@@H](Cc3ccc4cc(Cl)c(N)nc4c3)CC[C@]2(O)[C@H]1O]

Step 1: To a mixture of chloro(1,5-cyclooctadiene)iridium(I) dimer (18.10 mg, 0.035 mmol) and DPPE (28.0 mg, 0.070 mmol) in CH₂Cl₂ (3.01 mL) was added 4,4,5,5-tetramethyl-1,3,2-dioxaborolane (281 μl, 1.76 mmol) under N₂. The mixture was degassed and backfilled three times with N₂. After stirring for 20 minutes at 25° C., 7-((3aR,4R,5aR,8aR)-2,2-dimethyl-6-methylenehexahydrocyclopenta[2,3]furo[3,4-d][1,3]dioxol-4-yl)-4-methyl-7H-pyrrolo[2,3-d]pyrimidine (230 mg, 0.703 mmol) in CH₂Cl₂ (3.01 mL) was added to the mixture under N₂. The mixture was degassed and backfilled with N₂ three times and the resulting mixture was stirred at 25° C. for 15 h. The reaction was cooled to 0° C., quenched with 4 mL MeOH, and concentrated under reduced pressure. The resulting residue was purified by column chromatography on silica (10-100% EtOAc/DCM) to afford 7-((3aR,4R,5aR,8aR)-2,2-dimethyl-6-((4,4,5,5-tetramethyl-1,3,2-dioxaborolan-2-yl)methyl)hexahydrocyclopenta[2,3]furo[3,4-d][1,3]dioxol-4-yl)-4-methyl-7H-pyrrolo[2,3-d]pyrimidine as aresin. MS: 456 (M+1).

Step 2: To a vial charged with 7-bromo-3-chloroquinolin-2-amine (50.9 mg, 0.198 mmol), 7-((3aR,4R,5aR,8aR)-2,2-dimethyl-6-((4,4,5,5-tetramethyl-1,3,2-dioxaborolan-2-yl)methyl)hexahydrocyclopenta[2,3]furo[3,4-d][1,3]dioxol-4-yl)-4-methyl-7H-pyrrolo[2,3-d]pyrimidine (75 mg, 0.165 mmol), PdCl₂(dppf)-CH₂Cl₂Adduct (26.9 mg, 0.033 mmol) was added THF (2.75 mL), water (549 μl), and charged with thallium (I) ethoxide (35.0 μl, 0.49 mmol). The reaction was heated for 72 h at 65° C. The reaction was diluted with EtOAc, filtered, washed with brine, dried over sodium sulfate, filtered, and concentrated under reduced pressure. The residue was purified by column chromatography on silica (10-100% EtOAc/DCM) to afford 3-chloro-7-(((3aR,4R,5aR,8aR)-2,2-dimethyl-4-(4-methyl-7H-pyrrolo[2,3-d]pyrimidin-7-yl)hexahydrocyclopenta[2,3]furo[3,4-d][1,3]dioxol-6-yl)methyl)quinolin-2-amine which was used without further purification. MS: 506 (M+1).

Step 3: 3-chloro-7-(((3aR,4R,5aR,8aR)-2,2-dimethyl-4-(4-methyl-7H-pyrrolo[2,3-d]pyrimidin-7-yl)hexahydrocyclopenta[2,3]furo[3,4-d][1,3]dioxol-6-yl)methyl)quinolin-2-amine (10 mg, 0.012 mmol) was dissolved in CH₂Cl₂ (3 mL). A drop of water was added followed by TFA (0.457 mL, 5.93 mmol). The reaction was heated to 50° C. for 2 h and reaction was concentrated under reduced pressure and submitted for SFC resolution on (Whelk-O (R,R), 21×250 MeOH w/0.1% NH₄OH 35% modifier in CO₂) to afford (2R,3R,3aS,6R,6aR)-6-((2-amino-3-chloroquinolin-7-yl)methyl)-2-(4-methyl-7H-pyrrolo[2,3-d]pyrimidin-7-yl)hexahydro-3aH-cyclopenta[b]furan-3,3a-diol as a solid MS: 466 (M+1). ¹H NMR (500 MHz, DMSO-d₆) δ 8.64 (s, 1H), 8.14 (s, 1H), 7.80 (d, J=3.8 Hz, 1H), 7.58 (d, J=8.2 Hz, 1H), 7.32 (s, 1H), 7.10 (d, J=8.1 Hz, 1H), 6.77 (d, J=3.7 Hz, 1H), 6.66 (s, 2H), 6.01 (d, J=8.1 Hz, 1H), 5.47 (s, 1H), 5.31 (s, 1H), 4.33 (d, J=8.1 Hz, 1H), 3.92 (m, 1H), 2.86 (dd, J=13.9, 8.2 Hz, 1H), 2.71 (dd, J=13.9, 8.2 Hz, 1H), 2.66 (s, 3H), 2.35 (m, 1H), 2.12-1.93 (m, 2H), 1.92-1.81 (m, 1H), 1.60 (m, 1H).

### Example 132

### (3aS,4S,5R)-1-((2-amino-3-bromoquinolin-7-yl)methyl)-5-(4-amino-7H-pyrrolo[2,3-d]pyrimidin-7-yl)tetrahydro-1H-cyclopenta[c]furan-3a,4(3H)-diol

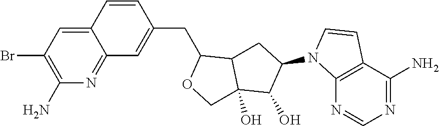 [SMILES: Nc1nc2cc(CC3OC[C@@]4(O)C3C[C@@H](n3ccc5c(N)ncnc53)[C@@H]4O)ccc2cc1Br]

Step 1: To a stirred solution of 3-bromo-7-iodo-N-(4-methoxybenzyl)quinolin-2-amine (3 g, 6.39 mmol) in THF (18.0 mL) was added allyltributylstannane (2.18 mL, 7.03 mmol) and Pd(PPh₃)₄ (0.739 g, 0.639 mmol) at room temperature under argon. The resulting mixture was heated to 95° C. and stirred for 6 h. The reaction was quenched by adding water (200 mL) and extracted by ethyl acetate (250 mL×2). The organic layer was washed with brine (200 mL), dried over sodium sulfate, filtered, and concentrated under reduced pressure. The residue was purified by column chromatography on silica (5-30% hexane/(3:1) mixture of ethyl acetate: ethanol) to afford 7-allyl-3-bromo-N-(4-methoxybenzyl)quinolin-2-amine as an oil. MS: 383/385 (M+1/M+3).

Step 2: Inside a round bottom flask equipped with a magnetic stir bar, 7-allyl-3-bromo-N-(4-methoxybenzyl)quinolin-2-amine (1 g, 2.61 mmol) was dissolved in DCM (52.2 mL). The flask was set in a dry ice-acetone bath and attached with an ozonator. Air was passed through ozonator and bubbled into the flask. The reaction was monitored every 5 minutes by LCMS, wherein the ozonator was stopped, and the reaction mixture was bubbled with air for 5 minutes before taking the aliquot. Upon complete consumption of starting material and observation of the corresponding ozonide mass, triphenylphosphane (1.369 g, 5.22 mmol) was added into the cold reaction and the reaction flask was taken out of the cold bath and stirred for another 30 minutes. The reaction was quenched with water (10 mL) extracted with DCM (10 mL), and the organic phase was washed with brine (10 mL). The organic layers were combined, dried over sodium sulfate, filtered, and concentrated under reduced pressure to afford 2-(3-bromo-2-((4-methoxybenzyl)amino)quinolin-7-yl)acetaldehyde which was used in next step without further purification.

Step 3: To a stirring solution of 2-(3-bromo-2-((4-methoxybenzyl)amino)quinolin-7-yl)acetaldehyde (5 g, 13.0 mmol) in THF (130 mL) was added vinylmagnesium bromide (20.8 mL, 1M in THF, 20.8 mmol) dropwise at 0° C. and stirred for 1 h. The reaction was quenched with saturated ammonium chloride solution (200 mL) and extracted with ethyl acetate (300 mL×2). The organic layer was washed with brine (200 mL), dried over sodium sulfate, filtered, and concentrated under reduced pressure. The residue was purified by column chromatography on silica (0-30% hexane/3:1 mixture of ethyl acetate: ethanol) to afford 1-(3-bromo-2-((4-methoxybenzyl)amino)quinolin-7-yl)but-3-en-2-ol. MS: 413/415 (M+1/M+3).

Step 4: The solution of 1-(3-bromo-2-((4-methoxybenzyl)amino)quinolin-7-yl)but-3-en-2-ol (1.5 g, 3.63 mmol) in anhydrous THF (36.3 mL) was cooled in an ice bath. Then sodium hydride (363 mg, 9.08 mmol) was added under an atmosphere of nitrogen. After stirring for 15 minutes, a solution of 3-bromoprop-1-yne (0.61 mL, 5.44 mmol) in toluene was added dropwise. The resulting reaction mixture was warmed to room temperature slowly overnight. The reaction was quenched with a saturated aqueous solution of ammonium chloride, and the mixture was extracted with ethyl acetate. The organic phase was separated, dried over magnesium sulfate, and concentrated under reduced pressure. The residue was purified by column chromatography on silica (0-30% hexane/3:1 mixture of ethylacetate:ethanol) to afford 3-bromo-N-(4-methoxybenzyl)-7-(2-(prop-2-yn-1-yloxy)but-3-en-1-yl)quinolin-2-amine as a solid. MS: 451/453 (M+1/M+3).

Step 5: To a vial charged with dicobalt octacarbonyl (80 mg, 0.233 mmol) was added a solution of 3-bromo-N-(4-methoxybenzyl)-7-(2-(prop-2-yn-1-yloxy)but-3-en-1-yl)quinolin-2-amine (700 mg, 1.55 mmol) in toluene (15.5 mL) under argon atmosphere. The reaction was heated to 100° C. in a CO Parr apparatus under −70 psi CO for 20 hours. The reaction was cooled to room temperature, diluted with diethyl ether, passed through a Celite plug, and then concentrated under reduced pressure to afford crude 3-((3-bromo-2-((4-methoxybenzyl)amino)quinolin-7-yl)methyl)-3a,4-dihydro-1H-cyclopenta[c]furan-5(3H)-one, which was used as is in next step without further purification.

Step 6: A solution of 3-((3-bromo-2-((4-methoxybenzyl)amino)quinolin-7-yl)methyl)-3a,4-dihydro-1H-cyclopenta[c]furan-5(3H)-one (500 mg, 1.04 mmol) in THF (20 mL) and MeOH (10 mL) was cooled to −40° C. in a dry ice/acetonitrile bath. Cerium(III) chloride heptahydrate (389 mg, 1.04 mmol) was added. The mixture was stirred cold for 20 minutes. Then sodium tetrahydroborate (79 mg, 2.09 mmol) was added. The reaction was vigorously stirred cold for 40 minutes. The reaction was removed from the bath and after a few minutes was quenched by pouring into a separatory funnel containing ethyl acetate (100 mL) and 3:2:1 saturated ammonium chloride:water:brine (70 mL). After extraction, the aqueous layer was washed again with ethyl acetate (100 mL×2). The combined organics were dried over sodium sulfate, filtered, and concentrated under reduced pressure. The residue was purified by silica gel flash column chromatography (0-50% hexane/3:1 mixture of ethyl acetate: ethanol) to afford 3-((3-bromo-2-((4-methoxybenzyl)amino)quinolin-7-yl)methyl)-3,3a,4,5-tetrahydro-1H-cyclopenta[c]furan-5-ol. MS: 481/483 (M+1/M+3).

Step 7: To a solution of 3-((3-bromo-2-((4-methoxybenzyl)amino)quinolin-7-yl)methyl)-3,3a,4,5-tetrahydro-1H-cyclopenta[c]furan-5-ol (140 mg, 0.291 mmol) in DCM (20 mL) was added pyridine (0.071 mL, 0.872 mmol), N,N-dimethylpyridin-4-amine (71.1 mg, 0.582 mmol), and di-tert-butyl dicarbonate (127 mg, 0.582 mmol). The reaction was stirred at room temperature overnight. The reaction was poured into a separatory funnel containing saturated ammonium chloride solution and DCM. After extraction, the organic layer was dried over sodium sulfate, filtered, and concentrated under reduced pressure. The resulting crude was purified by column chromatography on silica (0-5-10% EtOAc/hexanes) to afford 3-((3-bromo-2-((4-methoxybenzyl)amino)quinolin-7-yl)methyl)-3,3a,4,5-tetrahydro-1H-cyclopenta[c]furan-5-yl tert-butyl carbonate. MS: 581/583 (M+1/M+3).

Step 8: To a vial charged with N,N′-((1R,2R)-cyclohexane-1,2-diyl)bis(2-(diphenylphosphaneyl)benzamide) (3.56 mg, 5.16 μmol), tetrabutylammonium difluorotriphenylsilicate (9.28 mg, 0.017 mmol), 4-chloro-7H-pyrrolo[2,3-d]pyrimidine (5.28 mg, 0.034 mmol), and Pd₂dba₃ (1.575 mg, 1.720 μmol) was added anhydrous THF (1 mL). This mixture was stirred at room temperature for 15 minutes. Then a solution of 3-((3-bromo-2-((4-methoxybenzyl)amino)quinolin-7-yl)methyl)-3,3a,4,5-tetrahydro-1H-cyclopenta[c]furan-5-yl tert-butyl carbonate (10 mg, 0.017 mmol) in anhydrous THF (1 mL) was added. The reaction was stirred under argon at room temperature overnight. The mixture was poured into a separatory funnel containing water (10 mL) and extracted with ethyl acetate (15 mL×2). The combined organics were dried over magnesium sulfate, filtered, and concentrated under reduced pressure. The residue was purified by column chromatography on silica (0-50% EtOAc/hexanes) to afford 3-bromo-7-((5-(4-chloro-7H-pyrrolo[2,3-d]pyrimidin-7-yl)-3,5,6,6a-tetrahydro-1H-cyclopenta[c]furan-1-yl)methyl)-N-(4-methoxybenzyl)quinolin-2-amine. MS: 616/618 (M+1/M+3).

Step 9: To a solution of 3-bromo-7-((5-(4-chloro-7H-pyrrolo[2,3-d]pyrimidin-7-yl)-3,5,6,6a-tetrahydro-1H-cyclopenta[c]furan-1-yl)methyl)-N-(4-methoxybenzyl)quinolin-2-amine (160 mg, 0.259 mmol) in THF (2 mL) and Water (1 mL) were added 4-methylmorpholine 4-oxide (60.8 mg, 0.519 mmol) and a solution of osmium(VIII) oxide (407 μL, 0.052 mmol). The mixture was stirred at room temperature overnight. The reaction was quenched with 40% aqueous sodium bisulfite solution (3 mL) and stirred for 15 minutes, and then extracted with chloroform containing 25% isopropyl acetate (5 mL×2). The combined organic layers were washed with water (3 mL) and then with brine (3 mL), dried over sodium sulfate, and filtered. The filtrate was concentrated under reduced pressure to afford 1-((3-bromo-2-((4-methoxybenzyl)amino)quinolin-7-yl)methyl)-5-(4-chloro-7H-pyrrolo[2,3-d]pyrimidin-7-yl)tetrahydro-1H-cyclopenta[c]furan-3a,4(3H)-diol, which was used in next step without further purification.

Step 10: To a solution of 1-((3-bromo-2-((4-methoxybenzyl)amino)quinolin-7-yl)methyl)-5-(4-chloro-7H-pyrrolo[2,3-d]pyrimidin-7-yl)tetrahydro-1H-cyclopenta[c]furan-3a,4(3H)-diol (16 mg, 0.025 mmol) in DCM (5 mL) was added 4-methylbenzenesulfonic acid-monohydrate (14.0 mg, 0.074 mmol) and 2,2-dimethoxypropane (30 μL, 0.244 mmol) under an argon atmosphere. The reaction was stirred at room temperature overnight. The reaction was poured into a separatory funnel containing saturated aqueous ammonium chloride (10 mL) and extracted with DCM (10 mL×2). The organic layers were combined, dried over sodium sulfate, filtered, and concentrated under reduced pressure to afford 3-bromo-7-((4-(4-chloro-7H-pyrrolo[2,3-d]pyrimidin-7-yl)-2,2-dimethyltetrahydro-5H,8H-furo[3′,4′:1,5]cyclopenta[1,2-d][1,3]dioxol-6-yl)methyl)-N-(4-methoxybenzyl)quinolin-2-amine, which was used in the next step without further purification.

Step 11: To a vial charged with 3-bromo-7-((4-(4-chloro-7H-pyrrolo[2,3-d]pyrimidin-7-yl)-2,2-dimethyltetrahydro-5H,8H-furo[3′,4′:1,5]cyclopenta[1,2-d][1,3]dioxol-6-yl)methyl)-N-(4-methoxybenzyl)quinolin-2-amine (16 mg, 0.023 mmol) was added ammonia (1 mL, 7M in MeOH, 7.00 mmol). The vial was sealed, and the reaction was heated in the microwave for 4 hours at 140° C. The reaction was concentrated under reduced pressure, and the residue was purified by column chromatography on silica (10-60% hexane/3:1 mixture of ethyl acetate: ethanol). The product was further purified by SFC purification (MeOH w/0.1% NH₄OH, 35% modifier in CO₂) to afford 7-(((3aS,4R,8aS)-4-(4-amino-7H-pyrrolo[2,3-d]pyrimidin-7-yl)-2,2-dimethyltetrahydro-5H,8H-furo[3′,4′:1,5]cyclopenta[1,2-d][1,3]dioxol-6-yl)methyl)-3-bromo-N-(4-methoxybenzyl)quinolin-2-amine. MS: 671/673 (M+1/M+3).

Step 12: To a flask charged with 7-(((3aS,4R,8aS)-4-(4-amino-7H-pyrrolo[2,3-d]pyrimidin-7-yl)-2,2-dimethyltetrahydro-5H,8H-furo[3′,4′:1,5]cyclopenta[1,2-d][1,3]dioxol-6-yl)methyl)-3-bromo-N-(4-methoxybenzyl)quinolin-2-amine (2 mg, 2.16 μmol) was added TFA (121 μl, 1.57 mmol), and the reaction was stirred at 45° C. for 3 h. The crude was concentrated under reduced pressure, and the residue was purified by mass-triggered reverse phase HPLC (MeCN/water with 0.1% TFA modifier) to afford (3aS,4S,5R)-1-((2-amino-3-bromoquinolin-7-yl)methyl)-5-(4-amino-7H-pyrrolo[2,3-d]pyrimidin-7-yl)tetrahydro-1H-cyclopenta[c]furan-3a,4(3H)-diol as a solid as a TFA salt. MS: 511/513 (M+1/M+3). ¹H NMR (499 MHz, DMSO-d₆) δ 8.31 (s, 1H), 8.00 (s, 1H), 7.57 (d, J=8.2 Hz, 1H), 7.36 (s, 1H), 7.25 (d, J=3.6 Hz, 1H), 7.14 (d, J=6.9 Hz, 1H), 6.89 (s, 2H), 6.56 (s, 2H), 6.50 (d, J=3.5 Hz, 1H), 5.02 (d, J=7.0 Hz, 1H), 4.99 (s, 1H), 4.92-4.80 (m, 1H), 4.24 (dd, J=10.5, 7.1 Hz, 1H), 4.00 (q, J=6.7 Hz, 1H), 3.76 (q, J=9.3 Hz, 2H), 3.05 (dd, J=13.5, 7.6 Hz, 1H), 2.91 (dd, J=13.3, 6.3 Hz, 1H), 2.28-2.19 (m, 1H), 1.99-1.87 (m, 1H), 1.59-1.44 (m, 1H).

### Examples 133 and 134

### (2R,3R,3aS,6S,6aR)-6-((2-amino-3-bromoquinolin-7-yl)methyl)-2-(4-methyl-7H-pyrrolo[2,3-d]pyrimidin-7-yl)tetrahydrofuro[3,4-b]furan-3,3a(4H)-diol and

### (2R,3R,3aS,6R,6aR)-6-((2-amino-3-bromoquinolin-7-yl)methyl)-2-(4-methyl-7H-pyrrolo[2,3-d]pyrimidin-7-yl)tetrahydrofuro[3,4-b]furan-3,3a(4H)-diol

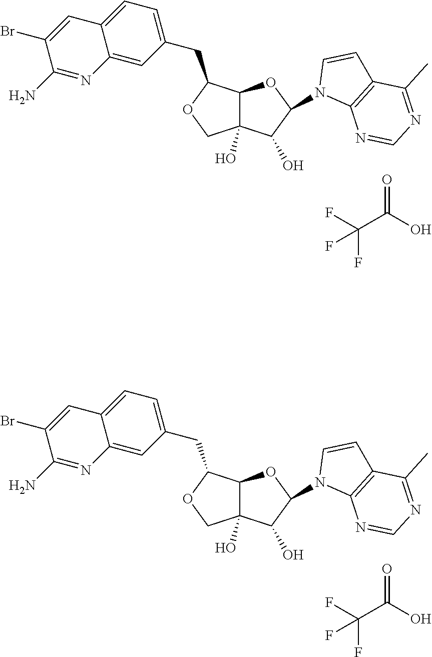

Step 1: DMP (61.6 g, 145 mmol) was added in portions to a solution of diacetone-D-glucose (25 g, 97 mmol) in DCM (300 mL) at 0° C. The reaction was warmed to room temperature and stirred overnight. The reaction was cooled to 0° C. and a saturated solution of sodium bicarbonate was added (100 mL) followed by a saturated solution of sodium sulfite (100 mL). The reaction was stirred for 30 minutes at room temperature, and the layers were separated. The aqueous layers were extracted with DCM (1×200 mL), the combined organic layers were then dried over sodium sulfate, filtered, and concentrated under reduced pressure to afford (3aR,5R,6aS)-5-((R)-2,2-dimethyl-1,3-dioxolan-4-yl)-2,2-dimethyldihydrofuro[2,3-d][1,3]dioxol-6(3aH)-one, which was used in the next step without further purification.

Step 2: A 500 mL 3-necked flask with stir bar, temperature probe, dropping funnel and septum was heated with a heat gun under vacuum, the glassware was cooled to room temperature and charged with (3aR,5R,6aS)-5-((R)-2,2-dimethyl-1,3-dioxolan-4-yl)-2,2-dimethyldihydrofuro[2,3-d][1,3]dioxol-6(3aH)-one (20 g, 77 mmol) and toluene (309 mL). The solution was cooled to 0° C. and vinylmagnesium chloride (58 mL, 1.6M, 93 mmol) was added dropwise at such a rate that the temperature did not exceed 5° C. After the addition was complete the reaction was warmed to room temperature and stirred for 3 h. The mixture was quenched with saturated aqueous ammonium chloride (250 mL) and then diluted with EtOAc (500 mL). The layers were separated, and the organic layer was washed with brine (2×250 mL), dried over sodium sulfate, filtered, and concentrated under reduced pressure to afford (3aR,5R,6R,6aR)-5-((R)-2,2-dimethyl-1,3-dioxolan-4-yl)-2,2-dimethyl-6-vinyltetrahydrofuro[2,3-d][1,3]dioxol-6-ol, which was used in the next step without further purification.

Step 3: A solution of (3aR,5R,6R,6aR)-5-((R)-2,2-dimethyl-1,3-dioxolan-4-yl)-2,2-dimethyl-6-vinyltetrahydrofuro[2,3-d][1,3]dioxol-6-ol (18.0 g, 62.8 mmol) in THF (165 mL) was cooled to 0° C. and sodium hydride (6.28 g, 157 mmol) was added in portions. The reaction was stirred for 30 minutes at 0° C. and then for 30 minutes at room temperature. Then TBAI (2.32 g, 6.28 mmol) was added followed by benzyl bromide (14.9 mL, 125 mmol), and the reaction was stirred at room temperature overnight. The mixture was cooled to 0° C., and quenched with a saturated ammonium chloride solution (200 mL). The mixture was diluted with EtOAc (200 mL) and the layers were separated. The combined organic layers were washed with brine (2×200 mL), dried over sodium sulfate, filtered, and concentrated under reduced pressure. The residue was purified by column chromatography on silica (0%-100% EtOAc/hexanes) to afford (3aR,5R,6R,6aR)-6-(benzyloxy)-5-((R)-2,2-dimethyl-1,3-dioxolan-4-yl)-2,2-dimethyl-6-vinyltetrahydrofuro[2,3-d][1,3]dioxole. ¹H NMR (600 MHz, CDCl₃) δ 7.43 (d, J=7.5 Hz, 2H), 7.37-7.32 (m, 2H), 7.31-7.26 (m, 1H), 5.89 (dd, J=18.0, 11.4 Hz, 1H), 5.85 (d, J=3.6 Hz, 1H), 5.49 (d, J=11.4 Hz, 1H), 5.32 (d, J=18.0 Hz, 1H), 4.71 (d, J=11.4 Hz, 1H), 4.65 (d, J=3.6 Hz, 1H), 4.63 (d, J=11.4 Hz, 1H), 4.34 (d, J=5.7 Hz, 1H), 4.19 (dd, J=5.9 Hz, 1H), 4.01-3.94 (m, 2H), 1.64 (s, 3H), 1.45 (s, 3H), 1.41 (s, 3H), 1.36 (s, 3H).

Step 4: A solution of (3aR,5R,6R,6aR)-6-(benzyloxy)-5-((S)-2,2-dimethyl-1,3-dioxolan-4-yl)-2,2-dimethyl-6-vinyltetrahydrofuro[2,3-d][1,3]dioxole (1000 mg, 2.66 mmol) and pyridine (0.645 mL, 7.97 mmol) in DCM (20 mL) was cooled to −78° C. and a stream of ozone (Triogen ozonator, using compressed air) was passed through the solution for 10 minutes. The vessel was purged with air and warmed to room temperature. The reaction was cooled to −78° C. and ozone was passed through for another 10-15 minutes. The vessel was purged with air and warmed to 0° C. The crude was diluted with MeOH (20 mL) and sodium borohydride (502 mg, 13.3 mmol) was added at 0° C. The reaction was stirred at this temperature for 3 h. The reaction was quenched by the addition of NaOH solution (1M, 50 mL), stirred for 5 minutes and diluted with EtOAc (200 mL). The organic layer was washed with brine (3×50 mL), dried over sodium sulfate, filtered, and concentrated under reduced pressure to afford ((3aR,5R,6R,6aR)-6-(benzyloxy)-5-((S)-2,2-dimethyl-1,3-dioxolan-4-yl)-2,2-dimethyltetrahydrofuro[2,3-d][1,3]dioxol-6-yl)methanol, which was used in the next step without further purification.

Step 5: Sulfuric acid (0.348 mL, 5% v/v aqueous solution, 6.53 mmol) was added to a solution of (3aR,5R,6R,6aR)-6-(benzyloxy)-5-((R)-2,2-dimethyl-1,3-dioxolan-4-yl)-2,2-dimethyl-6vinyltetrahydrofuro[2,3-d][1,3]dioxole (6.15 g, 16.3 mmol) in MeCN (30 mL) at room temperature, and the reaction was stirred for 2 h. The reaction was made basic with a minimal amount of sodium hydroxide and then magnesium sulfate was added. The slurry was filtered and concentrated under reduced pressure to afford (R)-1-((3aR,5R,6R,6aR)-6-(benzyloxy)-6-(hydroxymethyl)-2,2-dimethyltetrahydrofuro[2,3-d][1,3]dioxol-5-yl)ethane-1,2-diol which was used crude without further purification.

Step 6: A solution of sodium periodate (277 mg, 1.30 mmol) in water was cooled to 0° C., then a solution of (R)-1-((3aR,5R,6R,6aR)-6-(benzyloxy)-6-(hydroxymethyl)-2,2-dimethyltetrahydrofuro[2,3-d][1,3]dioxol-5-yl)ethane-1,2-diol (315 mg, 0.925 mmol) in MeOH (2 mL) was added dropwise. The reaction was stirred for 1 h at 0° C. and then warmed to room temperature and stirred overnight. The reaction was cooled to 0° C., and ethylene glycol (1 mL, 17.9 mmol) was added. The reaction was stirred for 5 minutes, then saturated sodium sulfite (50 mL) was added, and the reaction was warmed to room temperature. The mixture was diluted with EtOAc (100 mL), and the organic layer was washed with brine (3×200 mL), dried over sodium sulfate, filtered, and concentrated under reduced pressure to afford (3aR,4aS,7aR,7bR)-7a-(benzyloxy)-2,2-dimethylhexahydrofuro[3′,4′:4,5]furo[2,3-d][1,3]dioxol-5-ol which was used without further purification.

Step 7: Potassium tert-butoxide (183 mg, 1.63 mmol) was added in portions to a suspension of methyltriphenylphosphonium bromide (611 mg, 1.71 mmol) in THF (4 mL) at room temperature. The reaction was stirred at room temperature for 3 h, then the solution was cooled to 0° C., a solution of (3aR,4aS,7aR,7bR)-7a-(benzyloxy)-2,2dimethylhexahydrofuro[3′,4′:4,5]furo[2,3-d][1,3]dioxol-5-ol (251 mg, 0.814 mmol) was added, and the reaction was stirred for 2 h. The mixture was quenched with saturated aqueous ammonium chloride (10 mL), and the mixture was extracted with EtOAc (1×50 mL). The combined organic layers were washed with brine (2×50 mL), dried over sodium sulfate, filtered, and concentrated under reduced pressure. The residue was purified by column chromatography on silica (0%-100% EtOAc/hexanes) to afford ((3aR,5R,6R,6aR)-6-(benzyloxy)-2,2-dimethyl-5-vinyltetrahydrofuro[2,3-d][1,3]dioxol-6-yl)methanol. ¹H NMR (600 MHz, CDCl₃) δ 7.40 (d, J=7.3 Hz, 2H), 7.37-7.33 (m, 2H), 7.31-7.27 (m, 1H), 5.94 (ddd, J=17.2, 10.8, 5.3 Hz, 1H), 5.82 (d, J=3.9 Hz, 1H), 5.49 (dt, J=17.3, 1.6 Hz, 1H), 5.30 (dt, J=10.8, 1.5 Hz, 1H), 4.79 (d, J=11.1 Hz, 1H), 4.74 (d, J=11.1 Hz, 1H), 4.73-4.70 (m, 1H), 4.68 (d, J=3.9 Hz, 1H), 3.80 (d, J=12.1 Hz, 1H), 3.69 (d, J=12.1 Hz, 1H), 1.63 (s, 3H), 1.39 (s, 3H).

Step 8: To a vial charged with 3-bromo-N-(2,4-dimethoxybenzyl)-7-iodoquinolin-2-amine (1.62 g, 3.26 mmol), tris(dibenzylideneacetone)dipalladium(0) (75 mg, 0.08 mmol), sodium tert-butoxide (310 mg, 3.3 mmol) and bis(2-diphenylphosphinophenyl)ether (88 mg, 0.16 mmol) was added ((3aR,5R,6R,6aR)-6-(benzyloxy)-2,2-dimethyl-5-vinyltetrahydrofuro[2,3-d][1,3]dioxol-6-yl)methanol (500 mg, 1.63 mmol) in a solution in THF (8.16 mL). The reaction was heated to 65° C. overnight. The reaction was quenched with water and then extracted with DCM twice. The combined DCM layer were dried with Na₂SO₄ and concentrated under reduced pressure to afford 7-(((3aR,4aR,7aR,7bR)-7a-(benzyloxy)-2,2-dimethylhexahydrofuro[3′,4′:4,5]furo[2,3-d][1,3]dioxol-5-yl)methyl)-3-bromo-N-(2,4-dimethoxybenzyl)quinolin-2-amine. MS: 677/679 (M+1/M+3).

Step 9: Boron trichloride (0.79 mL, 0.79 mmol) was added to a solution of 7-(((3aR,4aR,7aR,7bR)-7a-(benzyloxy)-2,2-dimethylhexahydrofuro[3′,4′:4,5]furo[2,3-d][1,3]dioxol-5-yl)methyl)-3-bromo-N-(2,4-dimethoxybenzyl)quinolin-2-amine (107 mg, 0.16 mmol) in DCM (8 mL) at −78° C. The reaction was stirred for 15 minutes then warmed to 0° C. and stirred for 30 minutes. The reaction was quenched by the addition of THF and saturated aqueous NaHCO₃ (4:1). All the solvent was evaporated under reduced pressure, and the crude was purified by mass-triggered reverse phase HPLC (MeCN/water with 0.1% NH₄OH modifier) to afford (3R,3aS,6aR)-6-((2-amino-3-bromoquinolin-7-yl)methyl)tetrahydrofuro[3,4-b]furan-2,3,3a(4H)-triol. MS: 397/399 (M+1/M+3).

Step 10: To a vial containing (3R,3aS,6S,6aR)-6-((2-amino-3-bromoquinolin-7-yl)methyl)tetrahydrofuro[3,4-b]furan-2,3,3a(4H)-triol (20 mg, 0.050 mmol) in anhydrous acetonitrile (750 μl) was added 1,1′-(azodicarbonyl)dipiperidine (19 mg, 0.076 mmol) followed by tri-n-butylphosphine (20 μl, 0.08 mmol) at room temperature. The mixture was stirred for 1 h. In a separate oven-dried vial containing 4-methyl-7H-pyrrolo[2,3-d]pyrimidine (13 mg, 0.10 mmol) dissolved in dry DMF (250 μl) was added NaH (4.0 mg, 0.10 mmol). This mixture was stirred for 30 minutes at room temperature and was then added to the mixture described before originally containing the triol. The final reaction mixture was then stirred at room temperature overnight. The reaction mixture was purified by Prep-HPLC (MeCN/water with 0.1% TFA modifier) directly to afford two isomers: (2R,3R,3aS,6S,6aR)-6-((2-amino-3-bromoquinolin-7-yl)methyl)-2-(4-methyl-7H-pyrrolo[2,3-d]pyrimidin-7-yl)tetrahydrofuro[3,4-b]furan-3,3a(4H)-diol as a TFA salt. MS: 512/514 (M+1/M+3). ¹H NMR (600 MHz, DMSO-d₆) δ 8.96 (s, 1H), 8.80 (s, 1H), 8.00 (s, 1H), 7.77 (d, J=8.3 Hz, 1H), 7.55 (s, 1H), 7.41 (d, J=8.2 Hz, 1H), 7.07 (s, 1H), 6.25 (d, J=8.0 Hz, 1H), 4.37 (d, J=8.0 Hz, 1H), 4.32-4.30 (m, 1H), 4.14-4.08 (m, 1H), 3.99-3.95 (m, 1H), 3.42-3.36 (m, 1H), 3.08-3.03 (m, 2H), 2.82 (s, 3H) and (2R,3R,3aS,6R,6aR)-6-((2-amino-3-bromoquinolin-7-yl)methyl)-2-(4-methyl-7H-pyrrolo[2,3-d]pyrimidin-7-yl)tetrahydrofuro[3,4-b]furan-3,3a(4H)-diol as aTFA salt. (MS: 512/514 (M+1/M+3). ¹H NMR (600 MHz, DMSO-d₆) δ 8.85 (s, 1H), 8.75 (s, 1H), 7.92 (s, 1H), 7.77 (d, J=8.1 Hz, 1H), 7.54 (s, 1H), 7.38 (d, J=7.8 Hz, 1H), 6.97 (s, 1H), 6.21 (d, J=7.6 Hz, 1H), 4.46 (d, J=7.6 Hz, 1H), 4.32-4.25 (m, 1H), 4.23-4.20 (m, 1H), 3.99-3.97 (m, 1H), 3.88-3.86 (m, 1H), 3.13-3.04 (m, 2H), 2.76 (s, 3H).

### Example 135

### (2R,3R,3aS,6S,6aR)-6-((2-amino-3-bromoquinolin-7-yl)methyl)-2-(4-amino-5-methyl-7H-pyrrolo[2,3-d]pyrimidin-7-yl)tetrahydrofuro[3,4-b]furan-3,3a(4H)-diol

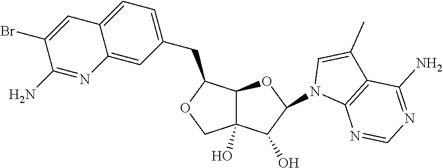 [SMILES: Cc1cn([C@@H]2O[C@@H]3[C@H](Cc4ccc5cc(Br)c(N)nc5c4)OC[C@]3(O)[C@H]2O)c2ncnc(N)c12]

Step 1: Crude 7-(((3aR,4aR,7aR,7bR)-7a-(benzyloxy)-2,2-dimethylhexahydrofuro[3′,4′:4,5]furo[2,3-d][1,3]dioxol-5-yl)methyl)-3-bromo-N-(2,4-dimethoxybenzyl)quinolin-2-amine (1.1 g, 1.6 mmol) was purified by column chromatography on silica (0-40% EtOAc/hexanes) to afford 7-(((3aR,4aR,5S,7aR,7bR)-7a-(benzyloxy)-2,2-dimethylhexahydrofuro[3′,4′:4,5]furo[2,3-d][1,3]dioxol-5-yl)methyl)-3-bromo-N-(2,4-dimethoxybenzyl)quinolin-2-amine as a solid. MS: 677/679 (M+1/M+3). ¹H NMR (600 MHz, CDCl₃) δ 8.03 (s, 1H), 7.65 (s, 1H), 7.44 (d, J=8.2 Hz, 1H), 7.40-7.27 (m, 6H), 7.18 (d, J=7.9 Hz, 1H), 6.52 (d, J=2.3 Hz, 1H), 6.47 (dd, J=8.3, 2.3 Hz, 1H), 6.06 (d, J=3.6 Hz, 1H), 5.85 (s, 1H), 4.76-4.73 (m, 2H), 4.70 (d, J=3.6 Hz, 1H), 4.53 (d, J=2.2 Hz, 1H), 4.49 (d, J=10.5 Hz, 1H), 4.27 (td, J=7.1, 2.2 Hz, 1H), 4.19-4.11 (m, 2H), 3.93 (d, J=10.3 Hz, 1H), 3.90 (s, 3H), 3.82 (s, 3H), 3.18-3.11 (m, 2H), 1.64 (s, 3H), 1.46 (s, 3H).

Step 2: Boron trichloride (5 mL, 5.1 mmol) was added to a solution of 7-(((3aR,4aR,5S,7aR,7bR)-7a-(benzyloxy)-2,2-dimethylhexahydrofuro[3′,4′:4,5]furo[2,3-d][1,3]dioxol-5-yl)methyl)-3-bromo-N-(2,4-dimethoxybenzyl)quinolin-2-amine (690 mg, 1.02 mmol) in DCM (20 mL) at −78° C. The reaction was stirred for 15 minutes then warmed to 0° C. and stirred for 30 minutes. The reaction was quenched by the addition of THF and saturated aqueous NaHCO₃ (8 mL: 2 mL). The mixture was concentrated under reduced pressure, and the residue was purified by reverse phase HPLC (MeCN/water with 0.1% NH₄OH modifier) to afford (3R,3aS,6S,6aR)-6-((2-amino-3-bromoquinolin-7-yl)methyl)tetrahydrofuro[3,4-b]furan-2,3,3a(4H)-triol. MS: 397/399 (M+1/M+3).

Step 3: To a vial containing (3R,3aS,6S,6aR)-6-((2-amino-3-bromoquinolin-7-yl)methyl)tetrahydrofuro[3,4-b]furan-2,3,3a(4H)-triol (20 mg, 0.05 mmol) in dry acetonitrile (750 μl) was added 1,1′-(azodicarbonyl)dipiperidine (19 mg, 0.076 mmol) followed by tri-n-butylphosphine (20 μl, 0.08 mmol) at room temperature. The mixture was stirred for 1 h. In a separate oven-dried vial containing 4-chloro-5-methyl-7H-pyrrolo[2,3-d]pyrimidine (17 mg, 0.1 mmol) in dry acetonitrile (250 μl) was added DBU (15 μl, 0.10 mmol). This mixture was stirred for 30 minutes at room temperature and was then added to the mixture described above originally containing the triol. The resulting mixture was stirred at room temperature overnight. The reaction was purified by reverse phase HPLC (MeCN/water with 0.1% TFA modifier) directly to afford (2R,3R,3aS,6S,6aR)-6-((2-amino-3-bromoquinolin-7-yl)methyl)-2-(4-chloro-5-methyl-7H-pyrrolo[2,3-d]pyrimidin-7-yl)tetrahydrofuro[3,4-b]furan-3,3a(4H)-diol as a TFA salt, as a solid. MS: 546/548 (M+1/M+3).

Step 4: Ammonia in methanol (2 mL, 7M, 14 mmol) was added to a microwave vial with (2R,3R,3aS,6S,6aR)-6-((2-amino-3-bromoquinolin-7-yl)methyl)-2-(4-chloro-5-methyl-7H-pyrrolo[2,3-d]pyrimidin-7-yl)tetrahydrofuro[3,4-b]furan-3,3a(4H)-diol, TFA salt (16 mg, 0.029 mmol). The reaction mixture was heated at 145° C. for 5 hrs. The reaction mixture was concentrated under reduced pressure, and the residue was purified by mass-triggered reverse phase HPLC (MeCN/water with 0.1% NH₄OH modifier) to afford (2R,3R,3aS,6S,6aR)-6-((2-amino-3-bromoquinolin-7-yl)methyl)-2-(4-amino-5-methyl-7H-pyrrolo[2,3-d]pyrimidin-7-yl)tetrahydrofuro[3,4-b]furan-3,3a(4H)-diol as a solid. MS: 527/529 (M+1/M+3). ¹H NMR (600 MHz, DMSO-d₆) δ 8.32 (s, 1H), 8.08 (s, 1H), 7.56 (d, J=8.3 Hz, 1H), 7.35 (s, 1H), 7.15-7.13 (m, 2H), 6.69 (s, 2H), 6.59 (s, 2H), 6.06 (d, J=8.2 Hz, 1H), 5.54 (d, J=6.7 Hz, 1H), 5.49 (s, 1H), 4.17-4.12 (m, 2H), 4.08-3.99 (m, 1H), 3.92 (d, J=9.0 Hz, 1H), 3.37-3.35 (m, 1H), 3.01-2.89 (m, 2H), 2.40 (s, 3H).

Example 136: Example 136 in Table 25 was synthesized in an analogously to steps 1-4 of Example 135 by substituting 4-chloro-5-methyl-7H-pyrrolo[2,3-d]pyrimidine with an appropriate nucleobase.

TABLE 25

| Ex | Structure | Name | MS |
|---|---|---|---|
| 136 | 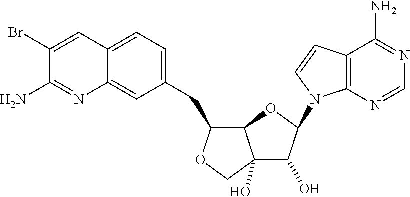 [SMILES: Nc1nc2cc(C[C@@H]3OC[C@@]4(O)[C@@H]3O[C@@H](n3ccc5c(N)ncnc53)[C@@H]4O)ccc2cc1Br] | (2R,3R,3aS,6S,6aR)-6-((2- amino-3-bromoquinolin-7- yl)methyl)-2-(4-amino-7H- pyrrolo[2,3-d]pyrimidin-7- yl)tetrahydrofuro[3,4-b]furan- 3,3a(4H)-diol | 513/515 (M + 1/M + 3) |

### Example 137

### (2R,3R,3aS,6S,6aR)-6-((2-amino-3-(difluoromethyl)quinolin-7-yl)methyl)-2-(4-amino-7H-pyrrolo[2,3-d]pyrimidin-7-yl)hexahydro-2H-cyclopenta[b]furan-3,3a-diol

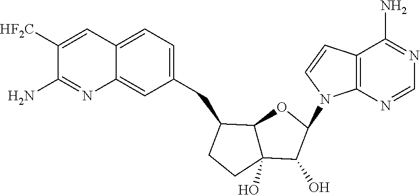

Step 1: A sealed tube was charged with (2R,3R,3aS,6aR)-2-(4-chloro-7H-pyrrolo[2,3-d]pyrimidin-7-yl)-6-methylenehexahydro-2H-cyclopenta[b]furan-3,3a-diol (200 mg, 0.650 mmol), 1,4-dioxane (3 mL) and concentrated ammonia hydrate (28 wt %, 3 mL) at room temperature. The mixture was sealed tightly and then stirred at 90° C. for 16 h. The reaction mixture was concentrated under reduced pressure, and the resulting residue was purified by column chromatography on silica (30% MeOH/DCM) to afford (2R,3R,3aS,6aR)-2-(4-amino-7H-pyrrolo[2,3-d]pyrimidin-7-yl)-6-methylenehexahydro-2H-cyclopenta[b]furan-3,3a-diol as an oil. MS: 289 (M+1). ¹H NMR (300 MHz, DMSO-d₆) δ 8.08 (s, 1H), 7.31 (d, J=3.9 Hz, 1H), 7.04 (br s, 2H), 6.63 (d, J=3.6 Hz, 1H), 6.06 (d, J=8.1 Hz, 1H), 5.37 (d, J=7.2 Hz, 1H), 5.25 (s, 1H), 5.10-5.06 (m, 2H), 4.31-4.23 (m, 2H), 2.65-2.61 (m, 1H), 2.50-2.40 (m, 1H), 2.07-2.02 (m, 1H), 1.72-1.61 (m, 1H).

Step 2: Compound (2R,3R,3aS,6aR)-2-(4-amino-7H-pyrrolo[2,3-d]pyrimidin-7-yl)-6-methylenehexahydro-2H-cyclopenta[b]furan-3,3a-diol (60 mg, 0.208 mmol) was co-evaporated with THF three times (2 mL each). Then it was treated with 9-BBN (2289 μl, 0.5M in THF, 1.15 mmol) at room temperature under argon. The mixture was stirred at 50° C. for 1 h. To this reaction was then added a solution of K₃PO₄ (220 mg, 1.04 mmol) in water (1 mL) at 0° C. and stirring continued at room temperature for 0.5 h. Then a solution of 7-bromo-3-(difluoromethyl)quinolin-2-amine (56.6 mg, 0.207 mmol) in THF (3 mL) was added followed by PdCl₂(dppf) (15.2 mg, 0.021 mmol). The final reaction mixture was irradiated with microwave radiation at 70° C. for 2 h. The mixture was concentrated under reduced pressure, and the resulting residue was purified by reverse phase column chromatography (ACN/water with 5 mM NH₄HCO₃ modifier). The product was further purified by reverse phase HPLC (ACN/water with 10 mM NH₄HCO₃ modifier) to afford (2R,3R,3aS,6S,6aR)-6-((2-amino-3-(difluoromethyl)quinolin-7-yl)methyl)-2-(4-amino-7H-pyrrolo[2,3-d]pyrimidin-7-yl)hexahydro-2H-cyclopenta[b]furan-3,3a-diol as a solid. MS: 483 (M+1). ¹H-NMR (400 MHz, DMSO-d₆) δ 8.17 (s, 1H), 8.09 (s, 1H), 7.67 (d, J=8.4 Hz, 1H), 7.45 (d, J=3.6 Hz, 1H), 7.29 (s, 1H), 7.26-7.00 (m, 4H), 6.68 (d, J=3.6 Hz, 1H), 6.45 (br s, 2H), 5.89 (d, J=8.0 Hz, 1H), 5.26 (d, J=6.8 Hz, 1H), 5.08 (s, 1H), 4.13 (t, J=7.6 Hz, 1H), 3.96 (d, J=5.6 Hz, 1H), 2.84-2.81 (m, 1H), 2.66-2.65 (m, 1H), 2.30-2.23 (m, 1H), 1.97-1.93 (m, 1H), 1.71-1.68 (m, 2H), 1.58-1.51 (m, 1H).

### Example 138

Example 138, as found in EP Application No. 15757502.8.

### N-(2-Hydroxy-3-(1,2,3,4-tetrahydroisoquinolin-3-yl)propyl)-4-(morpholine-4-carbonyl)benzamide (138)

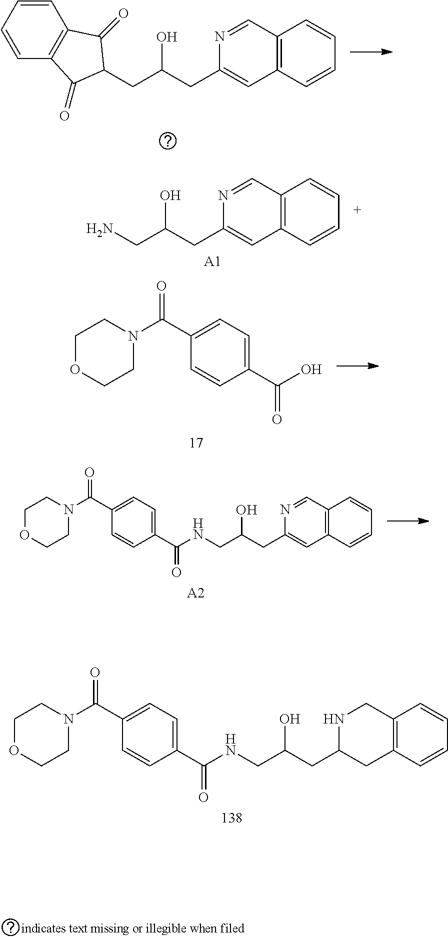

### (a) 1-Amino-3-(isoquinolin-3-yl)propan-2-ol (A1)

2-(2-Hydroxy-3-(isoquinolin-3-yl)propyl)isoindoline-1,3-dione (I3) (114 mg, 0.343 mmol) and absolute ethanol (5 mL) were brought to 80° C. Hydrazine hydrate (0.25 mL, 8.0 mmol) was added, and the mixture stirred at 80° C. After two hours, the heterogeneous mixture was cooled to room temperature, diluted with cold absolute ethanol (5 mL) and filtered. The collected solids were washed with cold ethanol (2 mL), and the combined filtrates were concentrated. The residue was dissolved in absolute ethanol and concentrated in vacuo three times to give the desired compound as a syrup (70 mg, quant.). The material was taken onto the next step without further purification. 1H NMR (400 MHz, d4-methanol) δ 9.19 (s, 1H), 8.22-8.16 (m, OH), 8.08-8.02 (m, 1H), 7.89-7.83 (m, 1H), 7.79-7.73 (m, 1H), 7.71 (s, 1H), 7.66-7.59 (m, 1H), 4.11-4.03 (m, 1H), 3.12-2.97 (m, 2H), 2.85-2.77 (m, 1H), 2.73-2.64 (m, 1H). LCMS-B: RT 1.55 min; m/z 203.2 [M+H]⁺.

### (b) N-(2-Hydroxy-3-(isoquinolin-3-yl)propyl)-4-(morpholine-4-carbonyl)benzamide (A2)

1-Amino-3-(isoquinolin-3-yl)propan-2-ol A1 (70 mg, 0.35 mmol), DMF (2.5 mL), DIPEA (0.121 mL, 0.69 mmol), 4-(morpholine-4-carbonyl)benzoic acid I7 (90 mg, 0.38 mmol) and HATU (197 mg, 0.52 mmol) were stirred at room temperature. After 3 hours, the mixture was added to 2% w/v sodium hydroxide (50 mL) and extracted with DCM (3×30 mL). The combined organic extracts were washed with brine (50 mL), dried over sodium sulfate and concentrated in vacuo. Silica gel chromatography (12 g silica cartridge, 0-10% methanol/DCM) gave the desired compound as a solid. 1H NMR (400 MHz, d4-methanol) δ 9.18 (s, 1H), 8.09-8.02 (m, 1H), 7.93-7.85 (m, 3H), 7.78-7.72 (m, 2H), 7.66-7.58 (m, 1H), 7.53-7.46 (m, 2H), 4.35-4.27 (m, 1H), 3.77 (br s, 3H), 3.66-3.57 (m, 3H), 3.55-3.47 (m, 2H), 3.22-3.14 (m, 1H), 3.11-3.04 (m, 1H); LCMS-B: RT 3.16 min; m/z 420.3 [M+H]⁺; m/z 418.1 [M−H]⁻.

### (c) N-(2-Hydroxy-3-(1,2,3,4-tetrahydroisoquinolin-3-yl)propyl)-4-(morpholine-4-carbonyl)benzamide (138)

N-(2-Hydroxy-3-(isoquinolin-3-yl)propyl)-4-(morpholine-4-carbonyl)benzamide A2 (20 mg, 0.048 mmol) and nickel(II) chloride hexahydrate (14 mg, 0.057 mmol) were dissolved in methanol (2 mL). The mixture was stirred at room temperature and sodium borohydride (22 mg, 0.57 mmol) was added in one portion. After 30 minutes, the mixture was quenched with 3M HCl (0.5 mL) and concentrated in vacuo. The residue was suspended in methanol and applied to a 1 g SCX cartridge. The cartridge was washed with methanol (15 mL) and eluted with 2M ammonia in methanol (15 mL). The basic eluate was concentrated in vacuo. The residue and nickel(II) chloride hexahydrate (14 mg, 0.057 mmol) were dissolved in methanol (2 mL). The mixture was stirred at room temperature and sodium borohydride (22 mg, 0.57 mmol) was added in one portion. After one hour the mixture was quenched with 3M HCl (0.5 mL) and concentrated in vacuo. The residue was suspended in methanol and applied to 2×1 g SCX cartridges. The cartridges were each washed with methanol (15 mL) and eluted with 2M ammonia in methanol (15 mL). The combined basic eluate was concentrated in vacuo to give the desired compound as a syrup (8 mg, 41%). LCMS-B: RT 3.18 min; m/z 424.3 [M+H]⁺.

Cartridges used are as follows:

| Manufacturer | Product |
|---|---|
| Biotage | ISOLUTE ® Phase Separator (3 mL unless otherwise |
| | stated) |
| Biotage | ISOLUTE ® SCX 1 g, (6 mL SPE Column unless |
| | otherwise stated) |
| Biotage | ISOLUTE ® SCX-2 1 g (6 mL Column) |
| Silicycle | SCX-bacti2 500 mg or 5 g |
| Agilent | Bond Elut ® SCX 10 g |
| Waters | Oasis ® HLB 35 cc (6 g) LP extraction cartridge |

### PRMT5-MEP50 Enzyme Methylation Assay

PRMT5-MEP50 biochemical assay is a direct measurement of the methylation activity of the enzyme complex on a short peptide substrate derived from the N-terminus of H4 histone. Methylation experiment is performed with recombinant PRMT5-MEP50 protein complex. The assessment of inhibitory effect of small molecules is measured by the effectiveness of the compounds to inhibit this reaction (EC₅₀).

In this assay, the potency (EC₅₀) of each compound was determined from a twenty-point (1:2 serial dilution; top compound concentration of 100000 nM) titration curve using the following outlined procedure. To each well of a white ProxiPlus 384 well-plate, 100 nL of compound (1% DMSO in final assay volume of 10 μL) was dispensed, followed by the addition of 8 μL of 1× assay buffer (50 mM Bicine pH 8.0, 1 mM DTT, 0.004% Tween20, 0.01% BSA) containing 1.25 nM of Full-length (FL)-PRMT5-MEP50 enzyme complex (recombinant proteins from baculovirus-transfected Sf21 cells: FL-PRMT5; MW=73837 kDa and FL-MEP50; MW=38614) and 1 μL of 150 μM S-(5′-Adenosyl)-L-Methionine Chloride (SAM). Plates were sealed and placed in a 37° C. humidified chamber for a 60 minutes pre-incubation with compound. Subsequently, each reaction was initiated by the addition of 1 μL 1× assay buffer containing 750 nM biotinylated H4R3(Mel) peptide. The final reaction in each well of 10 μL consists of 1.0 nM PRMT5-MEP50, 75 nM biotinylated-peptide, and 15 μM SAM. Methylation reactions proceeded for 150 minutes in a sealed plate at 37° C. Reactions were immediately quenched by the addition of 1 μL of 5% formic acid. Plates were then frozen and shipped to SAMDI™ Tech Inc. to determine the percent conversion from H4R3(Mel) to H4R3(Me2). Dose-response curves were generated by plotting percent effect (% product conversion; Y-axis) vs. Log 10 compound concentrations (X-axis). EC₅₀ values were determined by non-linear regression according to models for either sigmoidal (4 parameters) or biphasic (7 parameters) dose-response curves.

### PRMT5 Cell Target Engagement (TE) Assay

The PRMT5 TE assay is a biomarker assay for identifying compounds that inhibit symmetric dimethylation of arginine (SDMA) of PRMT5 substrates. The following substrates have been reported for PRMT5: Histone H2A and H4 R3, Histone H3 R2, Histone H3 R8, spliceosome Sm proteins, ribosomal protein RPS10, p53, FEN1, nucleoplasmin, nucleolin, EGFR and EBNA. The assay will focus on detecting symmetrically dimethylated nuclear proteins using high content imaging technology. Detection of the expression of symmetrically dimethylated nuclear proteins is through a mixture of primary rabbit monoclonal antibodies to SDMA (CST 13222), which in turn recognized by an Alexafluor 488 dye-conjugated anti-rabbit IgG secondary antibody. The IN Cell Analyzer 2200 or Opera-Phenix measures nuclear Alexafluor 488 fluorescent dye intensity that is directly related to the level of expression of symmetrically dimethylated nuclear proteins at the single cell level. Nuclear AF488 dye intensities are compared to the mean value for DMSO treated cells (MIN) to report percent of inhibition for each compound-treated well.

In this assay, the cell potency (EC50) of each compound was determined from a ten point (1:3 serial dilution; top compound concentration of 10000 nM) titration curve using the following outlined procedure. Each well of a BD falcon collagen coated black/clear bottom 384-well plate was seeded with 4000 MCF-7 cells in 30 μl media and allowed to attach for 5 hours. Media is ATCC-formulated Eagle's Minimum Essential Medium, Catalog No. 30-2003. The following components were added to the base medium: 0.01 mg/mL human recombinant insulin; fetal bovine serum to a final concentration of 10%. Additional 30 l of media containing 2× compounds were added to each well. Cells were treated for 3 days in 37° C. CO₂ incubator. On day 3, cells were fixed with Cytofix, permeabilized with 0.4% Triton-X-100/Cytofix, and washed with D-PBS without Ca/Mg. Cells were blocked with Licor Odessey blocking reagent for 1 hour at room temperature, followed by incubation with anti-SDMA (1:1000) antibody at 4° C. overnight. 1° antibody was removed, followed by three washings with DPBS without Ca/Mg and 0.05% Tween20. Hoechst (5 μg/mL), Cell Mask deep stain (1:2000) and Alexa488-conjugated goat anti-rabbit IgG (2 μg/mL) was added for 1 hour at room temperature. A final washing step (three washes) was performed before sealing plate for imaging on In Cell Analyzer 2200 or Opera-Phenix. Images from analyzer were uploaded to Columbus for image analysis. IC₅₀ values were determined by 4 parameters robust fit of percent fluorescence units vs. (Log 10) compound concentrations.

Representative compounds of the present invention were tested using the assay protocol described in this example. Results are provided in Table 26 below.

TABLE 26
When only one EC₅₀ is shown, the data was fit to a 4 parameters
single site sigmodal model. When two EC50s are shown,
the data was fit to a 7 parameters biphasic model

| | Enzyme Methylation | |
|---|---|---|
| | Assay (EC₅₀ or EC₅₀ | TE Assay |
| Ex. No. | 1, nM; EC₅₀ 2, nM) | (EC₅₀, nM) |
| 1 | 0.5; 56 | 18.0 |
| 2 | 0.5 | 0.7 |
| 3 | 0.8; 193 | 20.0 |
| 4 | 2.4; 288 | 186.0 |
| 5 | 0.8 | 19.0 |
| 6 | 0.6 | 1.2 |
| 7 | 0.7 | 4.8 |
| 8 | 0.5 | 0.5 |
| 9 | 0.3 | 1.3 |
| 10 | 0.5; 22 | 6.4 |
| 11 | 0.9; 501 | 131.0 |
| 12 | 33; 20890 | 10000.0 |
| 13 | 0.5; 89 | 14.0 |
| 14 | 1.3; 182 | 17.0 |
| 15 | 0.8 | 0.5 |
| 16 | 0.4 | 0.9 |
| 17 | 0.3 | 0.9 |
| 18 | 0.4; 228.5 | 10.6 |
| 19 | 0.4 | 1.1 |
| 20 | 0.5 | 18.6 |
| 21 | 0.8 | 0.5 |
| 22 | 1.0 | 30.0 |
| 23 | 0.4 | 1.5 |
| 24 | 0.4 | 1.3 |
| 25 | 11.0; 8913.0 | 258.2 |
| 26 | 1.1 | 4.0 |
| 27 | 0.4 | 2.8 |
| 28 | 0.8 | 13.0 |
| 29 | 0.3 | 1.7 |
| 30 | 0.7 | 0.8 |
| 31 | 0.4 | 0.8 |
| 32 | 0.4 | 0.8 |
| 33 | 0.6 | 0.4 |
| 34 | 0.3 | 0.3 |
| 35 | 0.5 | 0.8 |
| 36 | 0.5 | 1.4 |
| 37 | 0.3 | 0.6 |
| 38 | 0.3 | 0.6 |
| 39 | 0.3 | 0.3 |
| 40 | 0.4 | 0.7 |
| 41 | 0.7 | 2.9 |
| 42 | 0.3 | 0.3 |
| 43 | 0.4 | 1.0 |
| 44 | 0.4 | 8.8 |
| 45 | 0.3 | 1.2 |
| 46 | 0.5 | 16.8 |
| 47 | 0.2 | 1.4 |
| 48 | 0.5 | 1.7 |
| 49 | 0.5 | 13.0 |
| 50 | 0.3 | 0.7 |
| 51 | 1.2 | 2.8 |
| 52 | 0.5 | 75.5 |
| 53 | 0.3 | 4.5 |
| 54 | 0.3 | 8.1 |
| 55 | 0.5; 190.5 | 3.9 |
| 56 | 0.2 | 1.2 |
| 57 | 0.7; 142.9 | 2.1 |
| 58 | 0.4 | 1.0 |
| 59 | 0.6 | 4.6 |
| 60 | 0.4 | 1.5 |
| 61 | 0.5 | 3.2 |
| 62 | 0.5 | 3.7 |
| 63 | 0.4 | 5.6 |
| 64 | 0.3 | 16.2 |
| 65 | 0.2; 199.5 | 178.6 |
| 66 | 0.3 | 0.9 |
| 67 | 0.8 | 1.2 |
| 68 | 0.5 | 1.1 |
| 69 | 1.1 | 103.0 |
| 70 | 0.4 | 1.0 |
| 71 | 1.6 | 9.0 |
| 72 | 0.9 | 1.2 |
| 73 | 0.4 | 2.0 |
| 74 | 0.3 | 1.0 |
| 75 | 0.7 | 5.0 |
| 76 | 0.9 | 8.6 |
| 77 | 2.0; 242.7 | 185.7 |
| 78 | 13.3; 1.19 | 5993.0 |
| 79 | 2.9 | 52.5 |
| 80 | 2.9 | 97.0 |
| 81 | 0.7 | 3.9 |
| 82 | 0.7; 75.9 | 87.2 |
| 83 | 1.2 | 41.1 |
| 84 | 0.6 | 7.5 |
| 85 | 1.2; 245.5 | 32.4 |
| 86 | 0.5; 63.8 | 37.5 |
| 87 | 0.5 | 22.9 |
| 88 | 0.2 | 25.3 |
| 89 | 1.3 | 4.4 |
| 90 | 0.9 | 42.2 |
| 91 | 0.4 | 6.3 |
| 92 | 0.5 | 9.9 |
| 93 | 0.5; 109.6 | 3.5 |
| 94 | 0.8 | 5.3 |
| 95 | 2.8 | 47.0 |
| 96 | 0.8 | 11.1 |
| 97 | 0.4 | 1.3 |
| 98 | 3.4; 2570.0 | 271.7 |
| 99 | 1.7, 1884.0 | 202.4 |
| 100 | 0.6; 32.0 | 7.4 |
| 101 | 0.3 | 1.0 |
| 102 | 0.3 | 2.0 |
| 103 | 0.4; 40.7 | 12.4 |
| 104 | 3.8 | 7.0 |
| 105 | 0.3; 21.9 | 0.9 |
| 106 | 0.3 | 0.5 |
| 107 | 0.2 | 0.5 |
| 108 | 0.2 | 0.9 |
| 109 | 0.7 | 5.9 |
| 110 | 0.9 | 39.6 |
| 111 | 30.6; 2661.0 | 2330.0 |
| 112 | 0.6 | 4.0 |
| 113 | 1.1 | 1.8 |
| 114 | 1.4 | 4.4 |
| 115 | 0.4; 255.1 | 13.6 |
| 116 | 2.4; 660.7 | 53.7 |
| 117 | 1.0 | 8.5 |
| 118 | 3.2; 1585.0 | 103.4 |
| 119 | 1.0; 116.1 | 180.0 |
| 120 | 0.6 | 2.0 |
| 121 | 0.4 | 1.0 |
| 122 | 0.6 | 2.5 |
| 123 | 1.0 | 13.1 |
| 124 | 0.3 | 2.3 |
| 125 | 0.8 | 0.7 |
| 126 | 0.6 | 2.9 |
| 127 | 0.7 | 0.8 |
| 128 | 0.7 | 15.5 |
| 129 | 33.9; 31620.0 | 10000.0 |
| 130 | 0.6 | 134.9 |
| 131 | 0.6 | 21.3 |
| 132 | 3.9; 3311.0 | 236.3 |
| 133 | 0.8 | 2.7 |
| 134 | 3.5 | 35.8 |
| 135 | 0.4 | 0.6 |
| 136 | 0.5 | 0.4 |
| 137 | 0.5 | 0.8 |

### PRMT5 Biochemical Assay

The disclosed compounds, as an example of PRMT5 inhibitors may be tested for in vitro activity in the following assay: A histone H4 derived peptide is used as substrate (amino acid sequence: Ser-Gly-Arg-Gly-Lys-Gly-Gly-Lys-Gly-Leu-Gly-Lys-Gly-Gly-Ala-Lys-Arg-His-Arg-Lys-Val-NH₂). Full-length PRMT5 enzyme (NCBI Reference sequence NP_006100.2) was co-expressed with His₆-MEP50 in insect cells and purified via Nickel immobilized metal affinity and gel filtration chromatography (“the enzyme”).

The 6 μL assay reactions are run in Greiner brand black 384-well low volume plates. All reactions contained assay buffer (phosphate buffered saline, 0.01% (v/v) Tween-20, 0.01% (w/v) albumin from chicken egg white, 1 mM dithiothreitol, 200 nM peptide substrate, 1 μM S-Adenosyl methionine, and 15 ng/reaction enzyme, with the enzyme being omitted from negative control reactions. Compounds were added in a volume of 100 nL from dilution series prepared in DMSO, positive and negative control reactions receiving the same volume DMSO without compound. The plates were sealed with adhesive seals and incubated for 4 hours at 37° C. Reaction progress was measured using the Transcreener™ EPIGEN methyltransferase assay (BellBrook Labs, Madison, Wis.) as recommended by the manufacturer. To each reaction 2 μL detection mix were added, containing coupling enzymes, fluorescence polarisation tracer, and AMP antibody. Plates were incubated for 90 min before being read on a PerkinElmer EnVision™ plate reader in fluorescence polarisation mode. IC₅₀ values were obtained from the raw readings by calculating percent inhibition (% I) for each reaction relative to controls on the same plate (% I=(I−CN)/(CP−CN) where CN/CP are the averages of the negative/positive reactions, respectively), then fitting the % I data vs. compound concentration [I] to % I=(A+((B−A)/(1+((C/[I])\hat{ }D)))) where A is the lower asymptote, B is the upper asymptote, C is the IC₅₀ value, and D is the slope.

| Example Number | IC₅₀ (μM) |
|---|---|
| 138 | 4.686 |

### PRMT5 Biomarker Assay

The disclosed compounds, but not limited to, as an example of a PRMT5 inhibitor administered for treatment may be tested for potency to inhibit symmetrical dimethylation of arginine in the following assay:

The cell line TE11 was seeded at a density of 12,000 cells per well in 96 well tissue culture plates in DME medium and 10% foetal bovine serum, and allowed to adhere overnight under standard culture conditions (37° C., 5% CO₂). Compound dilutions prepared in DMSO were added to the medium, with negative control wells reserved for treatment with DMSO only and positive controls receiving a potent PRMT5 inhibitor. The concentration of the inhibitor had been previously determined to give maximum inhibition of the methylation. After incubation for 72 h, cells were washed twice in ice-cold PBS, lysed in lysis buffer (20 mM Tris pH 7.4, 135 mM NaCl, 1.5 mM MgCl₂, 1 mM EGTA, 10% glycerol and 1% Triton-X100), centrifuged at 15,000 xg and the supernatants collected for subsequent analysis. The methylation level was determined using the EpiQuik™ Global Di-Methyl Histone H4R3 Quantification ELISA Kit (Epigentek, Farmingdale, N.Y.) as per the manufacturer's recommendations; in parallel the total protein amount in the lysate was quantified using a Lowry protein assay. The methylation level was corrected for the total protein amount of each sample, normalised to the controls, and the data fitted against a four-parameter logistic model to determine the 50% inhibitory concentration (IC₅₀).

| Example Number | IC₅₀ (μM) |
|---|---|
| 138 | 0.956 |

### General Experimental Details for Compounds 138

1H NMR spectra were recorded on a Bruker Ultrashield Plus (400 MHz) or a Bruker AVANCE (400 MHz). The multiplicity of a signal is designated by the following abbreviations: s, singlet; d, doublet; t, triplet; q, quartet; dd, doublet of doublets; dt, doublet of triplets; tt, triplet of triplets; br, broad; m, multiplet. All observed coupling constants, J, are reported in Hertz.

LCMS data was generated using either an Agilent 6100 Series Single Quad LCMS (LCMS-A), an Agilent 1260 Infinity Series UPLC/MS (LCMS-B), an Agilent 1200 Series G6110A Quadrupole LCMS or Waters 2695 alliance (LCMS-C). Chlorine isotopes are reported as 35C1, Bromine isotopes are reported as either 79Br or 81Br or both 79Br/81Br.

### LCMS Method B (LCMS-B):

### Instrument: Agilent 1260 Infinity Series UPLC/MS

Pump: 1260 Infinity G1312B Binary pump

### Autosampler: 1260 Infinity G1367E 1260 HiP ALS

### Detector: 1290 Infinity G4212A 1290 DAD

LC conditions:
Reverse Phase HPLC analysis

### Column: Poroshell 120 EC-C18 2.7 μm 50×3.0 mm

Column temperature: 35° C.

### Injection Volume: 1 μL

### Solvent A: Water 0.1% Formic Acid

### Solvent B: MeCN 0.1% Formic Acid

Gradient: 5-100% solvent B over 3.8 min
Detection: monitored at 254 nm and 214 nm
MS conditions:

### Ion Source: Quadrupole

### Ion Mode: API-ES

Drying gas temp: 350° C.
Capillary voltage (V): 3000 (positive)
Capillary voltage (V): 3000 (negative)

### Scan Range: 100-1000

Step size: 0.1 sec
Acquisition time: 5 min
Flash chromatography was performed using a Biotage Isolera purification system using either Grace or RediSep® silica cartridges.
Column chromatography was performed using Tklst (China), grand grade, 100-200 meshes silica gel.

### BIOLOGIC EXAMPLES

DNA samples from the top 6 responding and the bottom 6 responding (non-responders) patient samples were submitted for targeted NGS (Cancer Genetics, Myeloid Panel). In addition, 26 fresh human AML patient samples obtained from two vendors, Discovery Life Sciences and ProteoGenex, were profiled against Example 138. Targeted NGS (custom panel) was performed and obtained the FLT3 ITD status of seach patient sample. The two experiments were aggregated such that a responder had an IC₅₀/LD₅₀-20 nM and a non-responder IC₅₀/LD₅₀>20 nM. The results were evaluated to determine if any mutations were correlated with response or non-response.

Data revealed that the commonly occurring FLT3 internal tandem duplication (ITD) and NPM1 mutations were significantly associated with response. Doublet and triplet combinations of FLT3 ITD, NPM1 mutation and DNMT3a mutation were also significantly associated with response. Additionally, mutations in TP53 were enriched in non-responding patient samples. Defined hotspot mutations in the RNA splicing genes SRSF2, ZRSR2, SF3B1 and are associated with responses to splicing inhibitors.

### Biologic Example 1

### Fresh Patient Sample Colony Forming Assay

Bacto-Agar (Becton Dickinson, TC grade) was prepared at 0.6% in H₂O and placed in a 47° C. water bath until required. The BFU-E-mix was made as follows: 100 ng/ml human GM-CSF (R&D Systems), 100 ng/ml human SCF (R&D Systems), 4 U/ml human EPO (Epoetin beta —‘NeoRecormon’ Roche, The Alfred Hospital Pharmacy), 100 ng/ml human IL3 (R&D Systems), 5 μM StemRegenin I (StemCell Technologies) and 0.35 μM UM171 (StemCell Technologies) in 5% FBS/PBS. 1 μl of 1000× concentration test compound was added per 35 mm non tissue culture grade sterile dish (Greiner Bio-one). Human patient bone marrow derived AML cells were counted and 5×10⁴ cells were mixed in 0.5 ml media (0.25 ml 2× AIMDM (Media Department of the Walter and Eliza Hall Institute of Medical Research) and 0.25 ml non-heat inactivated Fetal Bovine Serum), then mixed with 0.5 ml agar before plating in a 1 ml/35 mm dish containing 0.1 ml BFU-E mix). The agar dishes were allowed to fully set. The dishes were transferred to a 37° C., 5% CO₂, 100% humidity incubator for 2 weeks before counting colonies. LD50 values were determined by fitting these data to a nonlinear regression curve.

### Fresh Patient Sample Genomic DNA Extraction and Analysis

Genomic DNA was isolated from uncultured human patient bone marrow derived AML cells using the QIAamp DNA Blood Mini Kit according to the manufacturer's instructions (Qiagen) before determining the concentration of genomic DNA using Qubit and submitting for targeted next generation sequencing using the TruSight Myeloid Panel (54 genes, Illumina). FLT3 ITD status was determined by polymerase chain reaction (PCR) followed by separation by capillary electrophoresis.

### Biologic Example 2

### 3D AML Activity Assay

To culture AML patient's bone marrow samples, frozen commercial samples were thawed in 100% FBS, filtered through 100 μm Nylon mesh, counted, centrifuged and re-suspended at the density of 7×10⁴/ml in IMDM supplemented with the following components: 20% FBS, 620 μM CaCl₂, 1 μM sodium succinate, 1 μM hydrocortisone, 55 μM β-mercaptoethanol, antibiotic-antimycotic, 100 ng/ml human SCF, 50 ng/ml human FLT3L, 20 ng/ml human IL3, 20 ng/ml human GM-CSF, 100 ng/ml hM-CSF, 20 ng/ml human G-CSF, 20 ng/ml human IL7, 40 ng/ml human TPO, 1.5 U/ml human EPO and 100 ng/ml human RANKL (AML media). 10 volumes of 40% matrigel containing 333 μg/ml fibronectin and 666 μg/ml of collagen I (AML matrix) were added to re-suspended cells. After gentle mixing by pipetting up and down, cells in AML matrix were seeded in 384-well plate at the density of 80,000 cells per well. After matrix was solidified after an incubation at 37° C. for 1 h, 30 ul of AML media was added to each well.

Test compound was evaluated in a 15-point 3 fold dilution dose response curve starting with 10 μM. Specifically, 30 ul of AML media containing 2× dose ranges of Example 138 was added to cells embedded in 3D gel on day 2. On day 7, 20 μl of old media was removed and 30 μl of fresh AML media containing 1× drug was added. Cell viability was assessed by using CellTiter-Glo 3D after 11 days of culture (10 days of exposure to Example 138). IC₅₀ values were determined by fitting these data to a nonlinear regression curve.

DNA Extraction for tNGS

To extract DNA, uncultured bone marrow samples were lysed in ice-cold Ambion lysis/binding buffer (supplied in the mirVana miRNA isolation kit) and the lysate was applied to a QIAshredder spin column. After a spin at 4° C. for 3 min at 16,000×g, the supernatant was applied to the same QIAshredder spin column. After the 2nd spin, the lysate was transferred to an AllPrep DNA spin column (supplied in the AllPrep Qiagen kit). After centrifugation at room temperature for 1 min at 10,000×g, DNA is retained on the column. DNA was extracted according to manufacturer's instructions enclosed in the ALLPrep Qiagen Kit. Subsequently, DNA samples were submitted for next generation sequencing of 54 frequently occurring myeloid mutations and FLT3 ITD status was confirmed by Sanger sequencing (Focus:Myeloid Panel, Cancer Genetics Inc).

### Aggregated Results for Examples 1 and 2

The number of responders with FLT3 ITD or mutations in DNMT3A, NPM1, SRSF2 or ZRSR2 were tabulated

| | Responder with mutation |
|---|---|
| Mutated gene | (total 27 responders) |
| FLT3ITD | 9 |
| DNMT3A | 11 |
| NPM1 | 11 |
| SRSF2 | 4 |
| ZRSR2 | 1 |

### Biologic Example 3

### Evaluating Example 138 in SF3B1 Mutant (Colorectal Cancer) CRC Cell Line.

HCC-2998 cells were trypsinized and resuspended at a density of 3333 cells/ml in RPMI1640 supplemented with 10% fetal bovine serum (growth media) before plating 90 μl/well in a clear bottomed, black walled, 96 well cell culture plate and incubating overnight at 37° C. Example 138 was evaluated in a 9-point 3-fold dilution dose response. Compound dilutions were prepared in a master DMSO plate at 200× final concentrations in DMSO before diluting the DMSO master plate 20-fold with growth medium to make an intermediate plate at 10× final concentrations. Subsequently, 10 μl from the intermediate compound plate was added to the cells to achieve the final concentration range of 10 μM to 1.524 nM and the cells were incubated at 37° C. After 3 days, fresh aliquots of 10 μl of 10× concentrated Example 138 from a duplicate intermediate dilution plate and 90 μl of growth media were added to the cells, and the cells were incubated for a further 4 days at 37° C. Cell viability was determined using Cell Titer Glo (Promega) according to the manufacturer's instructions. Luminescent values were measured using Biotek Synergy 2 μlate reader before normalizing to 10 μM puromycin treated cells (100% cell death) and 0.5% DMSO (0% cell death). The IC50 value of Example 138 was determined by fitting these data to a nonlinear regression curve using GraphPad Prism 7.02 program. Example 138 inhibited the proliferation of SF3B1 mutant (R957Q) HCC2998 CRC cells with an IC50 value of 302 nM.

1. A method of identifying a patient diagnosed with cancer for treatment with a protein arginine N-methyltransferase 5 (PRMT5) inhibitor comprising:
a) obtaining a biological sample comprising cancer cells from a patient diagnosed with cancer;
b) measuring the gene expression level of a predictive biomarker of PRMT5 inhibitor responsiveness in the biological sample;
wherein the predictive biomarker is selected from:
i) a FLT3 internal tandem duplication; or
ii) a mutation in NPM1 or DNMT3a; or
iii) a mutation in any of the following splicing genes SRSF2, ZRSR2, or SF3B1;
c) identifying a patient predicted to be responsive to treatment with a PRMT5 inhibitor;
d) administering an effective amount of a PRMT5 inhibitor, or a pharmaceutically acceptable salt thereof, to the patient if the predictive biomarker is present.

2. The method of identifying a patient diagnosed with cancer for treatment with a protein arginine N-methyltransferase 5 (PRMT5) inhibitor according to claim 1, wherein the cancer is acute myeloid leukemia (AML) comprising:
a) obtaining a biological sample comprising cancer cells from a patient diagnosed with AML;
b) measuring the gene expression level of a predictive biomarker of PRMT5 inhibitor responsiveness in the biological sample;
c) wherein the predictive biomarker is selected from:
i) a FLT3 internal tandem duplication; or
ii) a mutation in NPM1 or DNMT3a; or
iii) a mutation in any of the following splicing genes SRSF2, ZRSR2, or SF3B1;
d) identifying a patient predicted to be responsive to treatment with a PRMT5 inhibitor;
e) administering an effective amount of a PRMT5 inhibitor, or a pharmaceutically acceptable salt thereof, to the patient, if the predictive biomarker is present.

3. The method of identifying a patient diagnosed with cancer predicted to be responsive to a treatment with protein arginine N-methyltransferase 5 (PRMT5) inhibitor according to claim 1, wherein the cancer is Myelodysplastic syndrome (MDS) comprising:
a) obtaining a biological sample comprising cancer cells from a patient diagnosed with MDS;
b) measuring the gene expression level of a predictive biomarker of PRMT5 inhibitor responsiveness in the biological sample;
c) wherein the predictive biomarker is selected from:
i) a FLT3 internal tandem duplication; or
ii) a mutation in NPM1 or DNMT3a; or
iii) a mutation in any of the following splicing genes SRSF2, ZRSR2, or SF3B1;
d) identifying a patient predicted to be responsive to treatment with a PRMT5 inhibitor;
e) administering an effective amount of a PRMT5 inhibitor, or a pharmaceutically acceptable salt thereof, to the patient, if the predictive biomarker is present.

4. The method of identifying a patient diagnosed with the cancer Myelodysplastic syndrome (MDS) predicted to be responsive to a treatment with protein arginine N-methyltransferase 5 (PRMT5) inhibitor according to claim 3, comprising:
a) obtaining a biological sample comprising cancer cells from a patient diagnosed with MDS;
b) measuring the gene expression level of a predictive biomarker of PRMT5 inhibitor responsiveness in the biological sample;
c) wherein the predictive biomarker is selected from:
i) mutation in DNMT3a; or
ii) mutation in any of splicing genes SRSF2, ZRSR2, or SF3B1; and
d) identifying a patient predicted to be responsive to treatment with a PRMT5 inhibitor;
e) administering an effective amount of a PRMT5 inhibitor, or a pharmaceutically acceptable salt thereof, to the patient, if the predictive biomarker is present.

5. A method for treating a PRMT5 associated cancer patient in need of treatment thereof, comprising the steps of:
a) obtaining a biological sample from the patient prior to administering a dose of a PRMT5 inhibitor;
b) measuring the samples for the presence of at least one of the following:
i) a FLT3 internal tandem duplication; or
ii) a mutation in NPM1 or DNMT3a; or
iii) a mutation in any of the following splicing genes SRSF2, ZRSR2, or SF3B1;
c) identifying a patient predicted to be responsive to treatment with a PRMT5 inhibitor;
d) administering to the patient a therapeutically effective amount of a PRMT5 inhibitor or a pharmaceutically acceptable salt thereof.

6. method of treatment of claim 5, wherein the cancer is acute myeloid leukemia or Myelodysplastic syndrome.

7. The method of claim 1, wherein the gene is NPM1 and the mutation is W288Cfs*12, L287fs, W290Sfs*5, or W288Cfs*7.

8. The method of claim 1, wherein the gene is DNMT3A and the mutation is R882C, R882H, R720H, Y592*, E229*, or V716D.

9. The method of claim 1, wherein the gene is SRSF2 and the mutation is P94_P95insR, P95H/L/R, P95T, P95fs, P95_R102del, or P107H

10. The method of claim 1, wherein the gene is ZRSR2 and the mutation is loss of function mutation.

11. The method of claim 1, wherein the gene is SF3B1 and the mutation is A284T, D586H, E592K, E622D, Y623C, R625C/G/H/L, N626D/I/S/Y, H662D/Q/Y, T663I, K666E/M/N/T/Q/R, K700E, V701F, I704F, G740E, K741T, G742D, D781G, E902K, or R957Q.

12. The method of claim 1, wherein the mutation is a FLT3 ITD.

13. The method of claim 1, and wherein the biological sample comprises a spliceosome alteration and the patient has MDS.

14. The method of claim 1, wherein the biological sample comprises a spliceosome alteration and the patient has AML.

15. (canceled)

16. A PRMT5 inhibitor for use in the treatment of cancer in a patient in need thereof, wherein the patient is defined by:
a) assaying a biological sample from a patient to determine if a patient has a predictive biomarker;
b) wherein the predictive biomarker is the presence of at least one of the following:
i) a FLT3 internal tandem duplication; or
ii) a mutation in NPM1 or DNMT3a; or
iii) a mutation in any of the following splicing genes SRSF2, ZRSR2, or SF3B1,
c) comprising administering a therapeutically effective amount of the PRMT5 inhibitor, or a pharmaceutically acceptable salt thereof, to the patient if the predictive biomarker is present.

17. (canceled)

18. (canceled)

19. (canceled)

20. (canceled)

21. (canceled)

22. A method of treating a patient diagnosed with cancer predicted to be responsive to a treatment with a protein arginine N-methyltransferase 5 (PRMT5) inhibitor after evaluating a biological sample from the patient for the presence of at least one of the following:
a) a FLT3 internal tandem duplication; or
b) a mutation in NPM1 or DNMT3A; or
c) a mutation in any of the following splicing genes SRSF2, ZRSR2, or SF3B1;
comprising administering an effective amount of a PRMT5 inhibitor, or a pharmaceutically acceptable salt thereof, to the patient, if the predictive biomarker is present.

23. The method of treatment of claim 22, wherein the cancer is acute myeloid leukemia or Myelodysplastic syndrome.

24. The method of claim 22, wherein the gene is NPM1 and the mutation is W288Cfs*12, L287fs, W290Sfs*5, or W288Cfs*7.

25. The method of claim 22, wherein the gene is DNMT3A and the mutation is R882C, R882H, R720H, Y592*, E229*, or V716D.

26. The method of claim 22, wherein the gene is SRSF2 and the mutation is P94_P95insR, P95H/L/R, P95T, P95fs, P95_R102del, or P107H.

27. The method of claim 22, wherein the gene is ZRSR2 and the mutation is loss of function mutation.

28. The method of claim 22, wherein the gene is SF3B1 and the mutation is A284T, D586H, E592K, E622D, Y623C, R625C/G/H/L, N626D/I/S/Y, H662D/Q/Y, T663I, K666E/M/N/T/Q/R, K700E, V701F, I704F, G740E, K741T, G742D, D781G, E902K, or R957Q.

29. The method of claim 22, wherein the mutation is a fms-related kinase 3 (FLT3) internal tandem duplication (ITD).

30. The method of claim 1, wherein the PRMT5 inhibitor is one of the following:
(1R,2S,3R,5R)-5-(((2-amino-3-bromoquinolin-7-yl)oxy)methyl)-3-(4-chloro-7H-pyrrolo[2,3-d]pyrimidin-7-yl)-1-methylcyclopentane-1,2-diol,
(1R,2S,3R,5R)-5-(((2-amino-3-bromoquinolin-7-yl)oxy)methyl)-3-(4-amino-7H-pyrrolo[2,3-d]pyrimidin-7-yl)-1-methylcyclopentane-1,2-diol,
(2R,3S,4R,5R)-2-(((2-amino-3-bromoquinolin-7-yl)oxy)methyl)-5-(4-amino-7H-pyrrolo[2,3-d]pyrimidin-7-yl)-3-ethynyltetrahydrofuran-3,4-diol,
(2R,3S,4R,5R)-2-(2-(2-amino-3-bromoquinolin-7-yl)ethyl)-5-(4-amino-7H-pyrrolo[2,3-d]pyrimidin-7-yl)-2,3-dimethyltetrahydrofuran-3,4-diol,
(2R,3R,3aS,6S,6aR)-6-((2-amino-3-bromoquinolin-7-yl)oxy)-2-(4-chloro-7H-pyrrolo[2,3-d]pyrimidin-7-yl)hexahydro-2H-cyclopenta[b]furan-3,3a-diol,
(2R,3R,3aS,6S,6aR)-6-((2-amino-3-bromoquinolin-7-yl)oxy)-2-(4-amino-7H-pyrrolo[2,3-d]pyrimidin-7-yl)hexahydro-2H-cyclopenta[b]furan-3,3a-diol,
(2R,3R,3aS,6S,6aR)-6-((2-amino-3-bromoquinolin-7-yl)oxy)-2-(4-methyl-7H-pyrrolo[2,3-d]pyrimidin-7-yl)hexahydro-2H-cyclopenta[b]furan-3,3a-diol,
(2R,3R,3aS,6S,6aR)-6-((2-amino-3-bromoquinolin-7-yl)methyl)-2-(4-methyl-7H-pyrrolo[2,3-d]pyrimidin-7-yl)hexahydro-2H-cyclopenta[b]furan-3,3a-diol,
(2R,3R,3aS,6S,6aR)-6-((2-amino-3-bromoquinolin-7-yl)methyl)-2-(4-amino-7H-pyrrolo[2,3-d]pyrimidin-7-yl)hexahydro-2H-cyclopenta[b]furan-3,3a-diol,
(2R,3S,4R,5R)-2-(((2-amino-3-bromoquinolin-7-yl)oxy)methyl)-5-(4-amino-7H-pyrrolo[2,3-d]pyrimidin-7-yl)-3-methyltetrahydrofuran-3,4-diol,
(2R,3S,4R,5R)-2-{[(2-amino-3-bromoquinolin-7-yl)oxy]methyl}-5-(4-chloro-7H-pyrrolo[2,3-d]pyrimidin-7-yl)-3-methyltetrahydrofuran-3,4-diol,
(2R,3S,4R,5R)-2-(((2-amino-3-bromoquinolin-7-yl)oxy)methyl)-5-(4-methoxy-7H-pyrrolo[2,3-d]pyrimidin-7-yl)-2-methyltetrahydrofuran-3,4-diol,
(2R,3S,4R,5R)-2-(((2-amino-3-bromoquinolin-7-yl)oxy)methyl)-5-(4-amino-7H-pyrrolo[2,3-d]pyrimidin-7-yl)-2-methyltetrahydrofuran-3,4-diol,
(2R,3S,4R,5R)-2-(((2-amino-3-bromoquinolin-7-yl)oxy)methyl)-5-(4-amino-7H-pyrrolo[2,3-d]pyrimidin-7-yl)-3-methyltetrahydrofuran-3,4-diol,
(1S,2R,3S,5R)-3-[2-(2-amino-3-bromo-7-quinolinyl)ethyl]-5-(4-amino-7H-pyrrolo[2,3-d]pyrimidin-7-yl)-3-methyl-1,2-cyclopentanediol,
(1S,2R,3aR,4S,6aR)-4-((2-amino-3-fluoroquinolin-7-yl)methyl)-2-(4-methyl-7H-pyrrolo[2,3-d]pyrimidin-7-yl)hexahydropentalene-1,6a(1H)-diol,
(1S,2R,3aR,4S,6aR)-4-((2-amino-3-chloroquinolin-7-yl)methyl)-2-(4-methyl-7H-pyrrolo[2,3-d]pyrimidin-7-yl)hexahydropentalene-1,6a(1H)-diol,
(1S,2R,3aR,4R,6aR)-4-((2-amino-3-fluoroquinolin-7-yl)methyl)-2-(4-methyl-7H-pyrrolo[2,3-d]pyrimidin-7-yl)hexahydropentalene-1,6a(1H)-diol,
(1S,2R,3aR,4S,6aR)-4-((2-amino-3-bromoquinolin-7-yl)methyl)-2-(4-methyl-7H-pyrrolo[2,3-d]pyrimidin-7-yl)hexahydropentalene-1,6a(1H)-diol,
(2R,3R,3aS,6S,6aR)-6-[(2-amino-3,8-difluoroquinolin-7-yl)methyl]-2-(4-amino-7H-pyrrolo[2,3-d]pyrimidin-7-yl)hexahydro-3aH-cyclopenta[b]furan-3,3a-diol,
(2R,3R,3aS,6S,6aR)-6-[(2-amino-3-chloro-5-fluoroquinolin-7-yl)methyl]-2-(4-amino-7H-pyrrolo[2,3-d]pyrimidin-7-yl)hexahydro-3aH-cyclopenta[b]furan-3,3a-diol,
(2R,3R,3aS,6S,6aR)-6-[(2-amino-3-chloro-8-fluoroquinolin-7-yl)methyl]-2-(4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-(difluoromethyl)quinolin-7-yl)methyl)-2-(4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3,5-difluoroquinolin-7-yl)methyl)-2-(4-amino-5-fluoro-7H-pyrrolo[2,3-d]pyrimidin-7-yl)hexahydro-3aH-cyclopenta[b]furan-3,3a-diol,
(2R,3R,3aS,6S,6aR)-6-((6-amino-7-fluoro-1,5-naphthyridin-3-yl)methyl)-2-(4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-chloro-8-fluoroquinolin-7-yl)methyl)-2-(4-amino-7H-pyrrolo[2,3-d]pyrimidin-7-yl)hexahydro-3aH-cyclopenta[b]furan-3,3a-diol,
(2R,3R,3aS,6S,6aR)-6-[(2-amino-3,6-difluoroquinolin-7-yl)methyl]-2-(4-amino-7H-pyrrolo[2,3-d]pyrimidin-7-yl)hexahydro-3aH-cyclopenta[b]furan-3,3a-diol,
(2R,3R,3aS,6S,6aR)-6-((7-amino-6-chloro-1,8-naphthyridin-2-yl)methyl)-2-(4-amino-7H-pyrrolo[2,3-d]pyrimidin-7-yl)hexahydro-3aH-cyclopenta[b]furan-3,3a-diol,
(2R,3R,3aS,6S,6aR)-6-[(2-amino-3,5-difluoroquinolin-7-yl)methyl]-2-(4-amino-7H-pyrrolo[2,3-d]pyrimidin-7-yl)hexahydro-3aH-cyclopenta[b]furan-3,3a-diol,
(1S,2R,3aR,4S,6aR)-4-((2-amino-3-fluoroquinolin-7-yl)methyl)-2-(4-methyl-7H-pyrrolo[2,3-d]pyrimidin-7-yl)hexahydropentalene-1,6a(1H)-diol,
(1S,2R,3aR,4S,6aR)-2-(4-amino-2-methyl-7H-pyrrolo[2,3-d]pyrimidin-7-yl)-4-((2-amino-3-chloroquinolin-7-yl)methyl)hexahydropentalene-1,6a(1H)-diol,
(1S,2R,3aR,4S,6aR)-2-(4-amino-2-methyl-7H-pyrrolo[2,3-d]pyrimidin-7-yl)-4-((2-amino-3-fluoroquinolin-7-yl)methyl)hexahydropentalene-1,6a(1H)-diol,
(1S,2R,3aR,4S,6aR)-4-((2-amino-3-fluoroquinolin-7-yl)methyl)-2-(4-amino-5-fluoro-7H-pyrrolo[2,3-d]pyrimidin-7-yl)hexahydropentalene-1,6a(1H)-diol,
(1S,2R,3aR,4S,6aR)-4-((2-amino-3-chloroquinolin-7-yl)methyl)-2-(4-amino-5-fluoro-7H-pyrrolo[2,3-d]pyrimidin-7-yl)hexahydropentalene-1,6a(1H)-diol,
(1S,2R,3aR,4S,6aR)-4-[(2-amino-3,5-difluoroquinolin-7-yl)methyl]-2-(4-amino-5-methyl-7H-pyrrolo[2,3-d]pyrimidin-7-yl)hexahydropentalene-1,6a(1H)-diol,
(1S,2R,3aR,4S,6aR)-4-[(2-amino-3-chloro-5-fluoroquinolin-7-yl)methyl]-2-(4-amino-5-methyl-7H-pyrrolo[2,3-d]pyrimidin-7-yl)hexahydropentalene-1,6a(1H)-diol,
(1S,2R,3aR,4S,6aR)-2-(4-amino-2-methyl-7H-pyrrolo[2,3-d]pyrimidin-7-yl)-4-((2-amino-3-bromoquinolin-7-yl)methyl)hexahydropentalene-1,6a(1H)-diol,
(1S,2R,3aR,4S,6aR)-4-((2-amino-3-fluoroquinolin-7-yl)methyl)-2-(4-amino-5-methyl-7H-pyrrolo[2,3-d]pyrimidin-7-yl)hexahydropentalene-1,6a(1H)-diol,
(1S,2R,3aR,4S,6aR)-4-((2-amino-3-chloroquinolin-7-yl)methyl)-2-(4-amino-5-methyl-7H-pyrrolo[2,3-d]pyrimidin-7-yl)hexahydropentalene-1,6a(1H)-diol,
(1S,2R,3aR,4S,6aR)-4-((2-amino-3-bromoquinolin-7-yl)methyl)-2-(4-amino-5-methyl-7H-pyrrolo[2,3-d]pyrimidin-7-yl)hexahydropentalene-1,6a(1H)-diol,
(1S,2R,3aR,4S,6aR)-4-((2-amino-3-bromoquinolin-7-yl)methyl)-2-(4-amino-7H-pyrrolo[2,3-d]pyrimidin-7-yl)hexahydropentalene-1,6a(1H)-diol,
(1S,2R,3aR,4S,6aR)-4-((2-amino-3-bromoquinolin-7-yl)methyl)-2-(4-amino-5-fluoro-7H-pyrrolo[2,3-d]pyrimidin-7-yl)hexahydropentalene-1,6a(1H)-diol,
(2R,3R,3aS,6S,6aR)-6-[(2-amino-3-bromoquinolin-7-yl)methyl]-2-[4-amino-5-(difluoromethyl)-7H-pyrrolo[2,3-d]pyrimidin-7-yl]hexahydro-3aH-cyclopenta[b]furan-3,3a-diol,
(2R,3R,3aS,6S,6aR)-6-((2-amino-3-fluoroquinolin-7-yl)methyl)-2-(4-amino-5-fluoro-2-methyl-7H-pyrrolo[2,3-d]pyrimidin-7-yl)hexahydro-3aH-cyclopenta[b]furan-3,3a-diol,
(2R,3R,3aS,6S,6aR)-2-(4-amino-2-fluoro-7H-pyrrolo[2,3-d]pyrimidin-7-yl)-6-((2-amino-3-fluoroquinolin-7-yl)methyl)hexahydro-3aH-cyclopenta[b]furan-3,3a-diol,
(2R,3R,3aS,6S,6aR)-6-((2-amino-3-bromoquinolin-7-yl)oxy)-2-(2-amino-7H-pyrrolo[2,3-d]pyrimidin-7-yl)hexahydro-3aH-cyclopenta[b]furan-3,3a-diol,
(2R,3R,3aS,6S,6aR)-2-(4-amino-2-chloro-7H-pyrrolo[2,3-d]pyrimidin-7-yl)-6-[(2-amino-3-chloroquinolin-7-yl)methyl]hexahydro-3aH-cyclopenta[b]furan-3,3a-diol,
(2R,3R,3aS,6S,6aR)-2-(4-amino-2-chloro-7H-pyrrolo[2,3-d]pyrimidin-7-yl)-6-[(2-amino-3-fluoroquinolin-7-yl)methyl]hexahydro-3aH-cyclopenta[b]furan-3,3a-diol,
(2R,3R,3aS,6S,6aR)-6-[(2-amino-3-bromoquinolin-7-yl)oxy]-2-(4-amino-5-phenyl-7H-pyrrolo[2,3-d]pyrimidin-7-yl)hexahydro-3aH-cyclopenta[b]furan-3,3a-diol,
(2R,3R,3aS,6S,6aR)-2-(4-amino-5-cyclopropyl-7H-pyrrolo[2,3-d]pyrimidin-7-yl)-6-[(2-amino-3-fluoroquinolin-7-yl)methyl]hexahydro-3aH-cyclopenta[b]furan-3,3a-diol,
(2R,3R,3aS,6S,6aR)-2-[4-amino-5-(difluoromethyl)-7H-pyrrolo[2,3-d]pyrimidin-7-yl]-6-[(2-amino-3-fluoroquinolin-7-yl)methyl]hexahydro-3aH-cyclopenta[b]furan-3,3a-diol,
(2R,3R,3aS,6S,6aR)-6-[(2-amino-3-bromoquinolin-7-yl)oxy]-2-(4-amino-5-cyclopropyl-7H-pyrrolo[2,3-d]pyrimidin-7-yl)hexahydro-3aH-cyclopenta[b]furan-3,3a-diol,
(2R,3R,3aS,6S,6aR)-6-[(2-amino-3-fluoroquinolin-7-yl)methyl]-2-(4-amino-7H-pyrrolo[2,3-d]pyrimidin-7-yl)-5,5-difluorohexahydro-3aH-cyclopenta[b]furan-3,3a-diol,
(2R,3R,3aS,5S,6S,6aR)-6-[(2-amino-3-fluoroquinolin-7-yl)methyl]-2-(4-amino-7H-pyrrolo[2,3-d]pyrimidin-7-yl)-5-fluorohexahydro-3aH-cyclopenta[b]furan-3,3a-diol,
(2R,3R,3aS,5S,6S,6aR)-2-(4-amino-5-fluoro-7H-pyrrolo[2,3-d]pyrimidin-7-yl)-6-[(2-amino-3-fluoroquinolin-7-yl)methyl]-5-fluorohexahydro-3aH-cyclopenta[b]furan-3,3a-diol,
(2R,3R,3aS,6S,6aR)-6-((2-amino-3-bromoquinolin-7-yl)oxy)-2-(4-amino-5-fluoro-7H-pyrrolo[2,3-d]pyrimidin-7-yl)hexahydro-3aH-cyclopenta[b]furan-3,3a-diol,
(2R,3R,3aS,6S,6aR)-2-(4-amino-7H-pyrrolo[2,3-d]pyrimidin-7-yl)-6-((2-((2,2,2-trifluoroethyl)amino)quinolin-7-yl)oxy)hexahydro-3aH-cyclopenta[b]furan-3,3a-diol,
(2R,3R,3aS,6S,6aR)-2-(4-amino-7H-pyrrolo[2,3-d]pyrimidin-7-yl)-6-((2-((cyclopropylmethyl)amino)quinolin-7-yl)oxy)hexahydro-3aH-cyclopenta[b]furan-3,3a-diol,
(2R,3R,3aS,6S,6aR)-6-((2-amino-3-fluoroquinolin-7-yl)methyl)-2-(4-amino-5-fluoro-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)oxy)-2-(4-amino-5-methyl-7H-pyrrolo[2,3-d]pyrimidin-7-yl)hexahydro-3aH-cyclopenta[b]furan-3,3a-diol,
(2R,3R,3aS,6S,6aR)-2-(4-amino-2-methyl-7H-pyrrolo[2,3-d]pyrimidin-7-yl)-6-((2-amino-3-bromoquinolin-7-yl)methyl)hexahydro-3aH-cyclopenta[b]furan-3,3a-diol,
(2R,3R,3aS,6S,6aR)-6-((2-amino-3-fluoroquinolin-7-yl)methyl)-2-(4-amino-5-methyl-7H-pyrrolo[2,3-d]pyrimidin-7-yl)hexahydro-3aH-cyclopenta[b]furan-3,3a-diol,
(2R,3R,3aS,6S,6aR)-2-(4-amino-2-methyl-7H-pyrrolo[2,3-d]pyrimidin-7-yl)-6-((2-amino-3-bromoquinolin-7-yl)oxy)hexahydro-3aH-cyclopenta[b]furan-3,3a-diol,
(2R,3R,3aS,6S,6aR)-6-((2-amino-3-chloroquinolin-7-yl)oxy)-2-(4-amino-5-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-fluoroquinolin-7-yl)oxy)-2-(4-amino-5-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-chloroquinolin-7-yl)methyl)-2-(4-amino-5-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-chloroquinolin-7-yl)methyl)-2-(4-amino-7H-pyrrolo[2,3-d]pyrimidin-7-yl)hexahydro-3aH-cyclopenta[b]furan-3,3a-diol,
(2R,3R,3aS,6S,6aR)-6-((2-amino-3-chloroquinolin-7-yl)methyl)-2-(4-amino-5-fluoro-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)oxy)-2-(4-amino-5-ethyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)methyl)-2-(4-amino-5-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-fluoroquinolin-7-yl)oxy)-2-(4-amino-7H-pyrrolo[2,3-d]pyrimidin-7-yl)hexahydro-3aH-cyclopenta[b]furan-3,3a-diol,
(2R,3R,3aS,6S,6aR)-6-((2-amino-3-chloroquinolin-7-yl)oxy)-2-(4-amino-7H-pyrrolo[2,3-d]pyrimidin-7-yl)hexahydro-3aH-cyclopenta[b]furan-3,3a-diol,
(2R,3R,3aS,6S,6aR)-6-((2-amino-3-fluoroquinolin-7-yl)oxy)-2-(4-amino-5-fluoro-7H-pyrrolo[2,3-d]pyrimidin-7-yl)hexahydro-3aH-cyclopenta[b]furan-3,3a-diol,
(2R,3R,3aS,6S,6aR)-6-((2-amino-3-chloroquinolin-7-yl)oxy)-2-(4-amino-5-fluoro-7H-pyrrolo[2,3-d]pyrimidin-7-yl)hexahydro-3aH-cyclopenta[b]furan-3,3a-diol,
(2R,3R,3aS,6S,6aR)-2-(4-amino-7H-pyrrolo[2,3-d]pyrimidin-7-yl)-6-((2,3-dihydro-1H-pyrrolo[2,3-b]quinolin-7-yl)oxy)hexahydro-3aH-cyclopenta[b]furan-3,3a-diol,
(2R,3R,3aS,6S,6aR)-6-((2-amino-3-(trifluoromethyl)quinolin-7-yl)methyl)-2-(4-amino-7H-pyrrolo[2,3-d]pyrimidin-7-yl)hexahydro-3aH-cyclopenta[b]furan-3,3a-diol,
(2R,3R,3aS,6S,6aR)-6-[(2-amino-3-bromoquinolin-7-yl)oxy]-2-[4-(hydroxymethyl)-7H-pyrrolo[2,3-d]pyrimidin-7-yl]hexahydro-3aH-cyclopenta[b]furan-3,3a-diol,
(2R,3R,3aS,6S,6aR)-6-[(2-amino-3-bromoquinolin-7-yl)oxy]-2-[4-(2-hydroxypropan-2-yl)-7H-pyrrolo[2,3-d]pyrimidin-7-yl]hexahydro-3aH-cyclopenta[b]furan-3,3a-diol,
(2R,3R,3aS,6S,6aR)-6-((2-amino-3-bromoquinolin-7-yl)oxy)-2-(4-(difluoromethyl)-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)oxy)-2-(2,4-dimethyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)methyl)-2-(2,4-dimethyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)oxy)-2-(4-amino-5-ethyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)methyl)-2-(4-isopropyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)methyl)-2-(5-fluoro-4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)oxy)-2-(7H-pyrrolo[2,3-d]pyrimidin-7-yl)hexahydro-3aH-cyclopenta[b]furan-3,3a-diol,
(2R,3R,3aS,6S,6aR)-6-((2-amino-3-fluoroquinolin-7-yl)oxy)-2-(4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)methyl)-2-(4-cyclopropyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-(trifluoromethyl)quinolin-7-yl)methyl)-2-(4-methyl-7H-pyrrolo[2,3-d]pyrimidin-7-yl)hexahydro-3aH-cyclopenta[b]furan-3,3a-diol,
(2R,3R,3aS,6S,6aR)-6-((2,3-dihydro-1H-pyrrolo[2,3-b]quinolin-7-yl)oxy)-2-(4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-fluoroquinolin-7-yl)methyl)-2-(2,4-dimethyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-chloroquinolin-7-yl)methyl)-2-(2,4-dimethyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)methyl)-2-(4-(methylamino)-7H-pyrrolo[2,3-d]pyrimidin-7-yl)hexahydro-3aH-cyclopenta[b]furan-3,3a-diol,
(1R,2S,3R,5R)-5-(((2-amino-3-bromoquinolin-7-yl)oxy)methyl)-1-methyl-3-(4-(methylamino)-7H-pyrrolo[2,3-d]pyrimidin-7-yl)cyclopentane-1,2-diol,
(2R,3R,3aS,6S,6aR)-6-((2-amino-3-bromoquinolin-7-yl)oxy)-2-(4-(methylamino)-7H-pyrrolo[2,3-d]pyrimidin-7-yl)hexahydro-3aH-cyclopenta[b]furan-3,3a-diol,
(2R,3R,3aS,6S,6aR)-6-((2-amino-3-fluoroquinolin-7-yl)methyl)-2-(4-(methylamino)-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)methyl)-2-(5-fluoro-4-(methylamino)-7H-pyrrolo[2,3-d]pyrimidin-7-yl)hexahydro-3aH-cyclopenta[b]furan-3,3a-diol,
(1S,2R,3aR,4S,6aR)-4-((2-amino-3-bromoquinolin-7-yl)methyl)-2-(4-(methylamino)-7H-pyrrolo[2,3-d]pyrimidin-7-yl)hexahydropentalene-1,6a(1H)-diol,
(2R,3S,4R,5R)-5-(4-amino-7H-pyrrolo[2,3-d]pyrimidin-7-yl)-2-methyl-2-((quinolin-7-yloxy)methyl)tetrahydrofuran-3,4-diol,
(2R,3S,4R,5R)-5-(4-amino-7H-pyrrolo[2,3-d]pyrimidin-7-yl)-3-methyl-2-((quinolin-7-yloxy)methyl)tetrahydrofuran-3,4-diol,
(1S,2R,3R,5R)-3-(((2-amino-3-bromoquinolin-7-yl)oxy)methyl)-5-(4-amino-7H-pyrrolo[2,3-d]pyrimidin-7-yl)-3-methylcyclopentane-1,2-diol,
(1S,2R,3R,5R)-5-(4-amino-7H-pyrrolo[2,3-d]pyrimidin-7-yl)-3-(((2-aminoquinolin-7-yl)oxy)methyl)-3-methylcyclopentane-1,2-diol,
(1S,2R,5R)-3-(2-(2-amino-3-bromoquinolin-7-yl)ethyl)-5-(4-amino-7H-pyrrolo[2,3-d]pyrimidin-7-yl)-3-(fluoromethyl)cyclopentane-1,2-diol,
(1R,2S,3R,5S)-5-(2-(2-amino-3-bromoquinolin-7-yl)ethyl)-3-(4-amino-7H-pyrrolo[2,3-d]pyrimidin-7-yl)-1-methylcyclopentane-1,2-diol,
(2R,3R,3aS,6S,6aR)-2-(4-amino-7H-pyrrolo[2,3-d]pyrimidin-7-yl)-6-((2-aminoquinolin-7-yl)oxy)hexahydro-2H-cyclopenta[b]furan-3,3a-diol,
(1R,2S,3R,5R)-5-(((2-aminoquinolin-7-yl)oxy)methyl)-1-methyl-3-(7H-pyrrolo[2,3-d]pyrimidin-7-yl)cyclopentane-1,2-diol,
(1R,2S,3R,5R)-5-(((2-amino-3-methylquinolin-7-yl)oxy)methyl)-1-methyl-3-(4-methyl-7H-pyrrolo[2,3-d]pyrimidin-7-yl)cyclopentane-1,2-diol,
(2R,3R,3aS,6S,6aR)-6-((2-aminoquinolin-7-yl)methyl)-2-(4-methyl-7H-pyrrolo[2,3-d]pyrimidin-7-yl)hexahydro-2H-cyclopenta[b]furan-3,3a-diol,
(2R,3R,3aS,6S,6aR)-6-((2-amino-3-bromoquinolin-7-yl)methyl)-2-(4-amino-5-fluoro-7H-pyrrolo[2,3-d]pyrimidin-7-yl)hexahydro-2H-cyclopenta[b]furan-3,3a-diol,
(1S,2R,3S,5R)-3-(2-(2-amino-3-bromoquinolin-7-yl)ethyl)-3-methyl-5-(4-methyl-7H-pyrrolo[2,3-d]pyrimidin-7-yl)cyclopentane-1,2-diol,
(1S,2R,3S,5R)-3-(2-(2,3-dihydro-1H-pyrrolo[2,3-b]quinolin-7-yl)ethyl)-3-methyl-5-(4-methyl-7H-pyrrolo[2,3-d]pyrimidin-7-yl)cyclopentane-1,2-diol,
(1R,2S,3R,5R)-5-(((2-amino-3-bromoquinolin-7-yl)oxy)methyl)-3-(4-amino-7H-pyrrolo[2,3-d]pyrimidin-7-yl)-1,5-dimethylcyclopentane-1,2-diol,
(2R,3R,3aS,6S,6aR)-6-((2-amino-3-chloroquinolin-7-yl)oxy)-2-(4-methyl-7H-pyrrolo[2,3-d]pyrimidin-7-yl)hexahydro-2H-cyclopenta[b]furan-3,3a-diol,
(2R,3R,3aS,6S,6aR)-6-((2-aminoquinolin-7-yl)oxy)-2-(4-methyl-7H-pyrrolo[2,3-d]pyrimidin-7-yl)hexahydro-2H-cyclopenta[b]furan-3,3a-diol,
(2R,3R,3aS,6S,6aR)-6-((2-amino-3-bromoquinolin-7-yl)oxy)-2-(2-amino-4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-(difluoromethyl)quinolin-7-yl)oxy)-2-(4-methyl-7H-pyrrolo[2,3-d]pyrimidin-7-yl)hexahydro-3aH-cyclopenta[b]furan-3,3a-diol,
(1S,2R,3R,5R)-3-(2-(2-amino-3-fluoroquinolin-7-yl)ethyl)-5-(4-amino-7H-pyrrolo[2,3-d]pyrimidin-7-yl)-3-methylcyclopentane-1,2-diol,
(1R,2S,3R,5S)-5-(2-(2-amino-3-bromoquinolin-7-yl)ethyl)-3-(4-amino-7H-pyrrolo[2,3-d]pyrimidin-7-yl)-1,5-dimethylcyclopentane-1,2-diol,
(1R,2S,3S,4R)-1-(2-(2-amino-3-bromoquinolin-7-yl)ethyl)-4-(4-amino-7H-pyrrolo[2,3-d]pyrimidin-7-yl)-2-methylcyclopentane-1,2,3-triol,
(1S,2R,3aR,4S,6aR)-4-((2-amino-3-bromoquinolin-7-yl)oxy)-2-(4-amino-7H-pyrrolo[2,3-d]pyrimidin-7-yl)hexahydropentalene-1,6a(1H)-diol,
(2R,3R,3aS,6S,6aR)-6-((2-amino-3-fluoroquinolin-7-yl)amino)-2-(4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-fluoroquinolin-7-yl)methyl)-2-(4-amino-7H-pyrrolo[2,3-d]pyrimidin-7-yl)-6a-methylhexahydro-3aH-cyclopenta[b]furan-3,3a-diol,
(2R,3R,3aS,6S,6aR)-6-((2-amino-3-chloroquinolin-7-yl)methyl)-2-(4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-fluoroquinolin-7-yl)methyl)-2-(4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-fluoroquinolin-7-yl)methyl)-2-(4-amino-7H-pyrrolo[2,3-d]pyrimidin-7-yl)hexahydro-3aH-cyclopenta[b]furan-3,3a-diol,
(2R,3R,3aS,6S,6aR)-6-((2-amino-3-methylquinolin-7-yl)methyl)-2-(4-amino-7H-pyrrolo[2,3-d]pyrimidin-7-yl)hexahydro-3aH-cyclopenta[b]furan-3,3a-diol,
(1S,2R,3S,5R)-5-(4-amino-7H-pyrrolo[2,3-d]pyrimidin-7-yl)-3-(2-(2,3-dihydro-1H-pyrrolo[2,3-b]quinolin-7-yl)ethyl)-3-methylcyclopentane-1,2-diol,
(2R,3R,3aS,6S,6aR)-6-((2-amino-3-bromoquinolin-7-yl)methyl)-2-(2-amino-4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-(difluoromethyl)quinolin-7-yl)oxy)-2-(4-amino-7H-pyrrolo[2,3-d]pyrimidin-7-yl)hexahydro-3aH-cyclopenta[b]furan-3,3a-diol,
(2R,3R,3aS,6R,6aR)-6-((2-amino-3-bromoquinolin-7-yl)oxy)-2-(4-methyl-7H-pyrrolo[2,3-d]pyrimidin-7-yl)hexahydro-3aH-cyclopenta[b]furan-3,3a-diol,
(2R,3R,3aS,6S,6aR)-6-((2-amino-3-bromoquinolin-7-yl)oxy)-2-(2-amino-5-fluoro-7H-pyrrolo[2,3-d]pyrimidin-7-yl)hexahydro-3aH-cyclopenta[b]furan-3,3a-diol,
(2R,3R,3aS,6R,6aR)-6-((2-amino-3-chloroquinolin-7-yl)methyl)-2-(4-methyl-7H-pyrrolo[2,3-d]pyrimidin-7-yl)hexahydro-3aH-cyclopenta[b]furan-3,3a-diol,
(3aS,4S,5R)-1-((2-amino-3-bromoquinolin-7-yl)methyl)-5-(4-amino-7H-pyrrolo[2,3-d]pyrimidin-7-yl)tetrahydro-1H-cyclopenta[c]furan-3a,4(3H)-diol,
(2R,3R,3aS,6S,6aR)-6-((2-amino-3-bromoquinolin-7-yl)methyl)-2-(4-methyl-7H-pyrrolo[2,3-d]pyrimidin-7-yl)tetrahydrofuro[3,4-b]furan-3,3a(4H)-diol,
(2R,3R,3aS,6R,6aR)-6-((2-amino-3-bromoquinolin-7-yl)methyl)-2-(4-methyl-7H-pyrrolo[2,3-d]pyrimidin-7-yl)tetrahydrofuro[3,4-b]furan-3,3a(4H)-diol,
(2R,3R,3aS,6S,6aR)-6-((2-amino-3-bromoquinolin-7-yl)methyl)-2-(4-amino-5-methyl-7H-pyrrolo[2,3-d]pyrimidin-7-yl)tetrahydrofuro[3,4-b]furan-3,3a(4H)-diol,
(2R,3R,3aS,6S,6aR)-6-((2-amino-3-bromoquinolin-7-yl)methyl)-2-(4-amino-7H-pyrrolo[2,3-d]pyrimidin-7-yl)tetrahydrofuro[3,4-b]furan-3,3a(4H)-diol, or
(2R,3R,3aS,6S,6aR)-6-((2-amino-3-(difluoromethyl)quinolin-7-yl)methyl)-2-(4-amino-7H-pyrrolo[2,3-d]pyrimidin-7-yl)hexahydro-2H-cyclopenta[b]furan-3,3a-diol,
N-(2-Hydroxy-3-(1,2,3,4-tetrahydroisoquinolin-3-yl)propyl)-4-(morpholine-4-carbonyl)benzamide, or a pharmaceutically acceptable salt thereof.
